(19) United States
(12) Patent Application Publication
(10) Pub. No.: US 2022/0098630 A1
(43) Pub. Date: Mar. 31, 2022

(54) CELL-FREE PRODUCTION OF ALLULOSE

(71) Applicant: GreenLight Biosciences, Inc., Medford, MA (US)

(72) Inventors: Daniel MacEachran, Medford, MA (US); Drew S. Cunningham, Winchester, MA (US); William Jeremy Blake, Winchester, MA (US); Matthew Eduardo Moura, Cambridge, MA (US)

(73) Assignee: GreenLight Biosciences, Inc., Medford, MA (US)

(21) Appl. No.: 17/415,640

(22) PCT Filed: Dec. 18, 2019
(86) PCT No.: PCT/US2019/067113

§ 371 Date: Jun. 17, 2021

Related U.S. Application Data
(60) Provisional application No. 62/784,314, filed on Dec. 21, 2018.

(51) Int. Cl. C12P 19/02; C12N 9/10; C12N 9/90; C12N 9/16; C12N 9/44; C12N 1/20; C12N 1/16; C12N 1/06; C12P 19/24

(52) U.S. Cl. CPC C12P 19/02; C12N 9/1051; C12N 9/90; C12N 9/16; C12N 9/2457; C12Y 302/01041; C12P 19/24; C12Y 302/01068; C12N 1/20; C12N 1/16; C12N 1/063; C12N 1/066; C12N 9/246

(57) ABSTRACT

Provided herein, in some embodiments, are cell-free systems, methods, kits, and compositions (e.g., cells and cell lysates) for converting a polysaccharide to allulose via the use of enzymes, such as thermostable enzymes.

### RELATED APPLICATION

This application claims the benefit under 35 U.S.C. § 119(e) of U.S. provisional application No. 62/784,314, filed Dec. 21, 2018, which is incorporated by reference herein in its entirety.

### BACKGROUND OF THE INVENTION

Existing technologies for the conversion of polysaccharides to simple sugars employ multiple biotransformations, with extensive purification processes following each step. While these processes are relatively inexpensive, owing to the application of immobilized enzymes and continuous production systems, the downstream processing dramatically impacts cost.

### SUMMARY OF THE INVENTION

Provided herein are cell-free systems, methods, compositions, and kits for the enzymatic conversion of polysaccharides to allulose. The methods and systems of the present disclosure implement sugar production pathways in cell-free reactions (e.g., a one-pot (consisting of multiple lysates) cell-free reaction system), to convert polysaccharides (e.g., maltodextrin) to allulose. Unlike processes that typically involve phosphorylation of substrates such as glucose to glucose 6-phosphate and employ high-energy phosphate sources such as ATP and phosphoenoylpyruvate, the processes described herein typically replace high energy phosphate sources with, for example, inexpensive inorganic phosphate (Pᵢ). An α-glucan phosphorylase is used to convert a polysaccharide to glucose 1-phosphate, which is then converted to glucose 6-phosphate via a phosphoglucomutase. Optionally, a debranching enzyme (an isoamylase or pullulanase) may be used to breakdown the polysaccharide through clevage of branched bonds, thus increasing substrate availability to the glucan phosphorylase. Subsequent enzymatic reaction(s) of a phosphoglucoismerase yields fructose 6-phosphate which is then converted to allulose 6-phosphate via an allulose 6-phosphate 3-epimerase (also referred to herein as allulose 6-phosphate epimerase, fructose 6-phosphate 3-epimerase, or fructose 6-phosphate epimerase). Finally, an allulose 6-phosphate phosphatase is used to create the final product, allulose (FIG. 1). Carbohydrate mass is transferred from a high-energy state in the starch-derived polysaccharide to the lower energy monosaccharide allulose. Thus, the reaction thermodynamics—phosphorylation of the substrate to dephosphorylation of the product—favor the product.

Further, the enzymatic process as a whole described herein is essentially irreversible, due to the last step of the pathway, in the conversion of allulose 6-phosphate to allulose via a specific allulose-6 phosphatase allowing for high yields of the desired allulose. By contrast, typical biotransformation methods for converting polysaccharides to allulose, for example, employ three distinct processes, two of which are reversible, with the final concentration of the product being governed by the thermodynamics of the enzymes being utilized. Starch, for example, is converted to glucose, glucose is isomerized to fructose, and fructose is epimerized to allulose. The isomerization of glucose to fructose has a yield of approximately 45%, thus significant downstream processing is required to yield a pure product and recycle uncatalyzed substrate. Similarly, the epimerization of fructose to allulose has a yield of approximately 20%, again requiring similar substantial downstream processing. The ability to directly transform polysaccharide to allulose in a cell-free system as described herein reduces costs by reducing downstream processing and loss of substrate.

Many of the enzymes that may be used in the processes provided herein are thermostable, which (1) enables thermal inactivation of deleterious activities contained within cellular lysates in which the conversion process is performed, and (2) decreases the chances of microbial contamination negatively impacting production runs. The enzymes of these conversion pathways can be isolated from thermophilic, mesophilic, or psychrophilic organisms and/or, in some embodiments, can be engineered to increase (or decrease) the thermostability or specificity of the enzymes. A thermophilic organism (thermophile) thrives at high temperatures, between 41° C. and 122° C. (106° F. and 252° F.). A mesophilic organism (mesophile) thrives at moderate temperatures, between 20° C. and 45° C. (68° F. and 113° F.). A psychrophilic organism (psychrophile) thrives at cold temperatures, between −20° C. and 10° C. (−4° F. and 50° F.).

Thus, some aspects of the present disclosure provide cell-free methods for producing allulose by converting a polysaccharide to glucose 1-phosphate using an α-glucan phosphorylase, converting glucose 1-phosphate to glucose 6-phosphate using a phosphoglucomutase, converting glucose 6-phosphate to fructose 6-phosphate using a phosphoglucoisomerase, converting fructose 6-phosphate to allulose 6-phosphate using an allulose-6 phosphate epimerase, and finally converting allulose 6-phosphate to allulose using an allulose 6-phosphate phosphatase (see FIG. 1).

Thus, some aspects of the present disclosure provide cell-free methods for producing allulose by converting cellulose/cellodextrin to glucose 1-phosphate using a cellodextrin phosphorylase, converting glucose 1-phosphate to glucose 6-phosphate using a phosphoglucomutase, converting glucose 6-phosphate to fructose 6-phosphate using a phosphoglucoisomerase, converting fructose 6-phosphate to allulose 6-phosphate using an allulose-6 phosphate epimerase, and finally converting allulose 6-phosphate to allulose using an allulose 6-phosphate phosphatase (see FIG. 2).

Some aspects of the present disclosure provide a cell-free method of producing glucose 1-phosphate from a polysaccharide, the method comprising converting a polysaccharide to glucose 1-phosphate using an α-glucan phosphorylase selected from the group consisting of AaGlgp (derived from Aquifex aeolicus), TzAgp (derived from Thermococcus zilligii), PtAgp (derived from Pseudothermotoga thermarum), Tm08495 (derived from Thermotoga Maritima), TcGlgP (derived from Thermus caldophilus) and PfAgp (derived from Pyrococcus furiosus). In some embodiments, the present disclosure provides a method of producing allulose comprising converting a polysaccharide to glucose 1-phosphate using an α-glucan phosphorylase selected from the group consisting of AaGlgp (derived from Aquifex aeolicus), TzAgp (derived from Thermococcus zilligii), PtAgp (derived from Pseudothermotoga thermarum), Tm08495 (derived from Thermotoga maritima), TcGlgP (derived from Thermus caldophilus) and PfAgp (derived from Pyrococcus furiosus). As will be appreciated by those of skill in the art, the other steps in the process from a polysaccharide to allulose would be carried out by other enzymes as described herein or in the art.

Some embodiments of the present disclosure provide a cell-free method of producing fructose 6-phosphate from glucose 6-phosphate, the method comprising converting glucose 6-phosphate to fructose 6-phosphate using a phosphoglucoisomerase selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis). In some embodiments, the present disclosure provides a method of producing allulose comprising converting glucose 6-phosphate to fructose 6-phosphate using a phosphoglucoisomerase selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis). As will be appreciated by those of skill in the art, the other steps in the process from a polysaccharide to allulose would be carried out by other enzymes as described herein or in the art.

Some embodiments of the present disclosure provide a cell-free method of producing fructose 6-phosphate from glucose 6-phosphate, the method comprising converting glucose 6-phosphate to fructose 6-phosphate using a phosphoglucoisomerase selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis). In some embodiments, the present disclosure provides a method of producing allulose comprising converting glucose 6-phosphate to fructose 6-phosphate using a phosphoglucoisomerase selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis). As will be appreciated by those of skill in the art, the other steps in the process from a cellulose/cellodextrin to allulose would be carried out by other enzymes as described herein or in the art.

Certain embodiments provide a cell lysate for producing allulose comprising an α-glucan phosphorylase selected from the group consisting of AaGlgp (derived from Aquifex aeolicus), TzAgp (derived from Thermococcus zilligii), PtAgp (derived from Pseudothermotoga thermarum), Tm08495 (derived from Thermotoga maritima), TcGlgP (derived from Thermus caldophilus) and PfAgp (derived from Pyrococcus furiosus), a phosphoglucomutase, a phosphoglucoisomerase, an allulose 6-phosphate epimerase, and an allulose 6-phosphate phosphatase, and optionally a debranching enzyme.

In other embodiments, the present disclosure provides a cell lysate for producing allulose comprising a phosphoglucoisomerase selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), an α-glucan phosphorylase, a phosphoglucomutase, an allulose 6-phosphate epimerase, an allulose 6-phosphate phosphatase, and optionally a debranching enzyme.

In other embodiments, the present disclosure provides a cell lysate for producing allulose comprising a phosphoglucoisomerase selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), a cellodextrin phosphorylase, a phosphoglucomutase, an allulose 6-phosphate epimerase, an allulose 6-phosphate phosphatase, and optionally a debranching enzyme.

Some aspects of the present disclosure provide a single cell lysate comprising an α-glucan phosphorylase selected from the group consisting of AaGlgp (derived from Aquifex aeolicus), TzAgp (derived from Thermococcus zilligii), PtAgp (derived from Pseudothermotoga thermarum), Tm08495 (derived from Thermotoga maritima), TcGlgP (derived from Thermus caldophilus) and PfAgp (derived from Pyrococcus furiosus), a phosphoglucomutase, a phosphoglucoisomerase, an allulose 6-phosphate epimerase, an allulose 6-phosphate phosphatase, and optionally a debranching enzyme.

Some embodiments of the present disclosure provide a single cell lysate comprising a phosphoglucoisomerase selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), an α-glucan phosphorylase, a phosphoglucomutase, an allulose 6-phosphate epimerase, an allulose 6-phosphate phosphatase, and optionally a debranching enzyme.

Some embodiments of the present disclosure provide a single cell lysate comprising a phosphoglucoisomerase selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), a cellodextrin phosphorylase, a phosphoglucomutase, an allulose 6-phosphate epimerase, an allulose 6-phosphate phosphatase, and optionally a debranching enzyme.

Certain embodiments disclose an engineered cell comprising an α-glucan phosphorylase selected from the group consisting of AaGlgp (derived from Aquifex aeolicus), TzAgp (derived from Thermococcus zilligii), PtAgp (derived from Pseudothermotoga thermarum), Tm08495 (derived from Thermotoga maritima), TcGlgP (derived from Thermus caldophilus) and PfAgp (derived from Pyrococcus furiosus), a phosphoglucomutase, a phosphoglucoisomerase, an allulose 6-phosphate epimerase, and an allulose 6-phosphate phosphatase, and optionally a debranching enzyme.

In other embodiments, the present disclosure provides an engineered cell comprising a phosphoglucoisomerase selected from the group consisting of CtPgi (derived from Clostridium thermocellum), TtPgi (derived from Thermus thermophilus), MjPgi (derived from Methanococcus jannaschii), PfPgi (derived from Pyrococcus furiosus), Ap0768 (derived from Aeropyrum pernix), and Cl1150 (derived from Caldisphaera lagunensis), an α-glucan phosphorylase, a phosphoglucomutase, an allulose 6-phosphate epimerase, an allulose 6-phosphate phosphatase, and optionally a debranching enzyme.

In other embodiments, the present disclosure provides an engineered cell comprising a phosphoglucoisomerase selected from the group consisting of CtPgi (derived from Clostridium thermocellum), TtPgi (derived from Thermus thermophilus), MjPgi (derived from Methanococcus jannaschii), PfPgi (derived from Pyrococcus furiosus), Ap0768 (derived from Aeropyrum pernix), and Cl1150 (derived from Caldisphaera lagunensis), a cellodextrin phosphorylase, a phosphoglucomutase, an allulose 6-phosphate epimerase, an allulose 6-phosphate phosphatase, and optionally a debranching enzyme.

Some aspects of the present disclosure provide an engineered cell comprising one or more enzymes selected from the group consisting of α-glucan phosphorylases selected from the group consisting of AaGlgp (derived from Aquifex aeolicus), TzAgp (derived from Thermococcus zilligii), PtAgp (derived from Pseudothermotoga thermarum), Tm08495 (derived from Thermotoga maritima), TcGlgP (derived from Thermus caldophilus) and PfAgp (derived from Pyrococcus furiosus), phosphoglucomutases, phosphoglucoisomerases, allulose 6-phosphate epimerases, allulose 6-phosphate phosphatases, and debranching enzymes.

Some embodiments of the present disclosure provide an engineered cell comprising one or more enzymes selected from the group consisting of phosphoglucoisomerases selected from the group consisting of CtPgi (derived from Clostridium thermocellum), TtPgi (derived from Thermus thermophilus), MjPgi (derived from Methanococcus jannaschii), PfPgi (derived from Pyrococcus furiosus), Ap0768 (derived from Aeropyrum pernix), and Cl1150 (derived from Caldisphaera lagunensis), α-glucan phosphorylases, phosphoglucomutases, allulose 6-phosphate epimerases, allulose 6-phosphate phosphatases, and debranching enzymes.

Some embodiments of the present disclosure provide an engineered cell comprising one or more enzymes selected from the group consisting of phosphoglucoisomerases selected from the group consisting of CtPgi (derived from Clostridium thermocellum), TtPgi (derived from Thermus thermophilus), MjPgi (derived from Methanococcus jannaschii), PfPgi (derived from Pyrococcus furiosus), Ap0768 (derived from Aeropyrum pernix), and Cl1150 (derived from Caldisphaera lagunensis), cellodextrin phosphorylases, phosphoglucomutases, allulose 6-phosphate epimerases, allulose 6-phosphate phosphatases, and debranching enzymes.

Certain embodiments disclose a mixture of cell lysates obtained from at least two cell populations, wherein the cells of each cell population express at least one enzyme selected from the group consisting of α-glucan phosphorylases selected from the group consisting of AaGlgp (derived from Aquifex aeolicus), TzAgp (derived from Thermococcus zilligii), PtAgp (derived from Pseudothermotoga thermarum), Tm08495 (derived from Thermotoga maritima), TcGlgP (derived from Thermus caldophilus) and PfAgp (derived from Pyrococcus furiosus), phosphoglucomutases, phosphoglucoisomerases, allulose 6-phosphate epimerases, allulose 6-phosphate phosphatases, and debranching enzymes.

In other embodiments, the present disclosure provides a mixture of cell lysates obtained from at least two cell populations, wherein the cells of each cell population express at least one enzyme selected from the group consisting of phosphoglucoisomerases selected from the group consisting of CtPgi (derived from Clostridium thermocellum), TtPgi (derived from Thermus thermophilus), MjPgi (derived from Methanococcus jannaschii), PfPgi (derived from Pyrococcus furiosus), Ap0768 (derived from Aeropyrum pernix), and Cl1150 (derived from Caldisphaera lagunensis), α-glucan phosphorylases, phosphoglucomutases, allulose 6-phosphate epimerases, allulose 6-phosphate phosphatases, and debranching enzymes.

In other embodiments, the present disclosure provides a mixture of cell lysates obtained from at least two cell populations, wherein the cells of each cell population express at least one enzyme selected from the group consisting of phosphoglucoisomerases selected from the group consisting of CtPgi (derived from Clostridium thermocellum), TtPgi (derived from Thermus thermophilus), MjPgi (derived from Methanococcus jannaschii), PfPgi (derived from Pyrococcus furiosus), Ap0768 (derived from Aeropyrum pernix), and Cl1150 (derived from Caldisphaera lagunensis), cellodextrin phosphorylases, phosphoglucomutases, allulose 6-phosphate epimerases, allulose 6-phosphate phosphatases, and debranching enzymes.

Some aspects of the present disclosure provide a reaction mixture of cell lysates comprising an α-glucan phosphorylase selected from the group consisting of AaGlgp (derived from Aquifex aeolicus), TzAgp (derived from Thermococcus zilligii), PtAgp (derived from Pseudothermotoga thermarum), Tm08495 (derived from Thermotoga maritima), TcGlgP (derived from Thermus caldophilus) and PfAgp (derived from Pyrococcus furiosus), a phosphoglucomutase, a phosphoglucoisomerase, an allulose 6-phosphate epimerase, an allulose 6-phosphate phosphatase, optionally a debranching enzyme, a polysaccharide, glucose 1-phosphate, glucose 6-phosphate, fructose 6-phosphate, allulose 6-phosphate, and allulose.

Some aspects of the present disclosure provide a reaction mixture of cell lysates comprising an α-glucan phosphorylase selected from the group consisting of AaGlgp (derived from Aquifex aeolicus), TzAgp (derived from Thermococcus zilligii), PtAgp (derived from Pseudothermotoga thermarum), Tm08495 (derived from Thermotoga maritima), TcGlgP (derived from Thermus caldophilus) and PfAgp (derived from Pyrococcus furiosus), a phosphoglucomutase, a phosphoglucoisomerase, an allulose 6-phosphate epimerase, an allulose 6-phosphate phosphatase, optionally a debranching enzyme, optionally a polysaccharide, optionally glucose 1-phosphate, optionally glucose 6-phosphate, optionally fructose 6-phosphate, optionally allulose 6-phosphate, and optionally allulose.

Some embodiments of the present disclosure provide a reaction mixture of cell lysates comprising an α-glucan phosphorylase, a phosphoglucomutase, a phosphoglucoisomerase selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), an allulose 6-phosphate epimerase, an allulose 6-phosphate phosphatase, optionally a debranching enzyme, a polysaccharide, glucose 1-phosphate, glucose 6-phosphate, fructose 6-phosphate, allulose 6-phosphate, and allulose.

Some embodiments of the present disclosure provide a reaction mixture of cell lysates comprising an α-glucan phosphorylase, a phosphoglucomutase, a phosphoglucoisomerase selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), an allulose 6-phosphate epimerase, an allulose 6-phosphate phosphatase, optionally a debranching enzyme, optionally a polysaccharide, optionally glucose 1-phosphate, optionally glucose 6-phosphate, optionally fructose 6-phosphate, optionally allulose 6-phosphate, and optionally allulose.

Some embodiments of the present disclosure provide a reaction mixture of cell lysates comprising a cellodextrin phosphorylase, a phosphoglucomutase, a phosphoglucoisomerase selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), an allulose 6-phosphate epimerase, an allulose 6-phosphate phosphatase, optionally a debranching enzyme, cellulose/cellodextrin, glucose 1-phosphate, glucose 6-phosphate, fructose 6-phosphate, allulose 6-phosphate, and allulose.

Some embodiments of the present disclosure provide a reaction mixture of cell lysates comprising a cellodextrin phosphorylase, a phosphoglucomutase, a phosphoglucoisomerase selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), an allulose 6-phosphate epimerase, an allulose 6-phosphate phosphatase, optionally a debranching enzyme, optionally cellulose, optionally cellodextrin, optionally glucose 1-phosphate, optionally glucose 6-phosphate, optionally fructose 6-phosphate, optionally allulose 6-phosphate, and optionally allulose.

Certain embodiments disclose a cell lysate mixture comprising an α-glucan phosphorylase selected from the group consisting of AaGlgp (derived from Aquifex aeolicus), TzAgp (derived from Thermococcus zilligii), PtAgp (derived from Pseudothermotoga thermarum), Tm08495 (derived from Thermotoga maritima), TcGlgP (derived from Thermus caldophilus) and PfAgp (derived from Pyrococcus furiosus), a phosphoglucomutase, a phosphoglucoisomerase, an allulose 6-phosphate epimerase, an allulose 6-phosphate phosphatase, optionally a debranching enzyme, a polysaccharide, glucose 1-phosphate, glucose 6-phosphate, fructose 6-phosphate, allulose 6-phosphate, and allulose for the synthesis of allulose.

Certain embodiments disclose a cell lysate mixture comprising an α-glucan phosphorylase selected from the group consisting of AaGlgp (derived from Aquifex aeolicus), TzAgp (derived from Thermococcus zilligii), PtAgp (derived from Pseudothermotoga thermarum), Tm08495 (derived from Thermotoga maritima), TcGlgP (derived from Thermus caldophilus) and PfAgp (derived from Pyrococcus furiosus), a phosphoglucomutase, a phosphoglucoisomerase, an allulose 6-phosphate epimerase, an allulose 6-phosphate phosphatase, optionally a debranching enzyme, optionally a polysaccharide, optionally glucose 1-phosphate, optionally glucose 6-phosphate, optionally fructose 6-phosphate, optionally allulose 6-phosphate, and optionally allulose for the synthesis of allulose.

In other embodiments, the present disclosure provides a cell lysate mixture comprising an α-glucan phosphorylase, a phosphoglucomutase, a phosphoglucoisomerase selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), an allulose 6-phosphate epimerase, an allulose 6-phosphate phosphatase, optionally a debranching enzyme, a polysaccharide, glucose 1-phosphate, glucose 6-phosphate, fructose 6-phosphate, allulose 6-phosphate, and allulose for the synthesis of allulose.

In other embodiments, the present disclosure provides a cell lysate mixture comprising an α-glucan phosphorylase, a phosphoglucomutase, a phosphoglucoisomerase selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), an allulose 6-phosphate epimerase, an allulose 6-phosphate phosphatase, optionally a debranching enzyme, optionally a polysaccharide, optionally glucose 1-phosphate, optionally glucose 6-phosphate, optionally fructose 6-phosphate, optionally allulose 6-phosphate, and optionally allulose for the synthesis of allulose.

In other embodiments, the present disclosure provides a cell lysate mixture comprising a cellodextrin phosphorylase, a phosphoglucomutase, a phosphoglucoisomerase selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), an allulose 6-phosphate epimerase, an allulose 6-phosphate phosphatase, optionally a debranching enzyme, cellulose/cellodextrin, glucose 1-phosphate, glucose 6-phosphate, fructose 6-phosphate, allulose 6-phosphate, and allulose for the synthesis of allulose.

In other embodiments, the present disclosure provides a cell lysate mixture comprising a cellodextrin phosphorylase, a phosphoglucomutase, a phosphoglucoisomerase selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), an allulose 6-phosphate epimerase, an allulose 6-phosphate phosphatase, optionally a debranching enzyme, optionally cellulose, optionally cellodextrin, optionally glucose 1-phosphate, optionally glucose 6-phosphate, optionally fructose 6-phosphate, optionally allulose 6-phosphate, and optionally allulose for the synthesis of allulose.

Some aspects of the present disclosure provide a cell-free method for producing allulose, the method comprising (a) culturing cells engineered to express a α-glucan phosphorylase selected from the group consisting of AaGlgp (derived from Aquifex aeolicus), TzAgp (derived from Thermococcus zilligii), PtAgp (derived from Pseudothermotoga thermarum), Tm08495 (derived from Thermotoga maritima), TcGlgP (derived from Thermus caldophilus) and PfAgp (derived from Pyrococcus furiosus), a phosphoglucomutase, a phosphoglucoisomerase, an allulose 6-phosphate epimerase, and an allulose 6-phosphate phosphatase to produce cultured cells that have expressed the enzymes; (b) lysing the cultured cells to produce a cell lysate; and (c) incubating the cell lysate in the presence of a polysaccharide and inorganic phosphate to produce allulose.

Some embodiments of the present disclosure provide a cell-free method for producing allulose, the method comprising (a) culturing cells engineered to express a α-glucan phosphorylase, a phosphoglucomutase, a allulose 6-phosphate epimerase, an allulose 6-phosphate phosphatase, and a phosphoglucoisomerase selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis) to produce cultured cells that express the enzymes; (b) lysing the cultured cells to produce a cell lysate; and (c) incubating the cell lysate in the presence of a polysaccharide and inorganic phosphate to produce allulose.

Some embodiments of the present disclosure provide a cell-free method for producing allulose, the method comprising (a) culturing cells engineered to express a cellodextrin phosphorylase, a phosphoglucomutase, a allulose 6-phosphate epimerase, an allulose 6-phosphate phosphatase, and a phosphoglucoisomerase selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis) to produce cultured cells that express the enzymes; (b) lysing the cultured cells to produce a cell lysate; and (c) incubating the cell lysate in the presence of cellulose/cellodextrin and inorganic phosphate to produce allulose.

Certain embodiments disclose a cell-free method for producing allulose, the method comprising (a) culturing at least two cell populations, wherein cells of each population are engineered to express at least one enzyme selected from the group consisting of α-glucan phosphorylases selected from the group consisting of AaGlgp (derived from Aquifex aeolicus), TzAgp (derived from Thermococcus zilligii), PtAgp (derived from Pseudothermotoga thermarum), Tm08495 (derived from Thermotoga maritima), TcGlgP (derived from Thermus caldophilus) and PfAgp (derived from Pyrococcus furiosus), phosphoglucomutases, phosphoglucoisomerases, allulose 6-phosphate epimerases, allulose 6-phosphate phosphatases, and optionally debranching enzymes to produce at least two cultured populations of cells expressing different enzymes; (b) lysing cells of the at least two cultured populations to produce at least two cell lysates; (c) combining at least two cell lysates to produce a cell lysate mixture that comprises an α-glucan phosphorylase, a phosphoglucomutase, a phosphoglucoisomerase, an allulose 6-phosphate epimerase, an allulose 6-phosphate phosphatase, and optionally a debranching enzyme; and (d) incubating the cell lysate mixture in the presence of a polysaccharide and inorganic phosphate to produce allulose.

In other embodiments, the present disclosure provides a cell-free method for producing allulose, the method comprising (a) culturing at least two cell populations, wherein cells of each population are engineered to express at least one enzyme selected from the group consisting of α-glucan phosphorylases, phosphoglucomutases, allulose 6-phosphate epimerases, allulose 6-phosphate phosphatases, phosphoglucoisomerases selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), and optionally debranching enzymes to produce at least two cultured populations of cells expressing different enzymes; (b) lysing cells of the at least two cultured populations to produce at least two cell lysates; (c) combining the at least two cell lysates to produce a cell lysate mixture that comprises an α-glucan phosphorylase, a phosphoglucomutase, a phosphoglucoisomerase, an allulose 6-phosphate epimerase, an allulose 6-phosphate phosphatase, and optionally a debranching enzyme; and (d) incubating the cell lysate mixture in the presence of a polysaccharide and inorganic phosphate to produce allulose.

In other embodiments, the present disclosure provides a cell-free method for producing allulose, the method comprising (a) culturing at least two cell populations, wherein cells of each population are engineered to express at least one enzyme selected from the group consisting of cellodextrin phosphorylases, phosphoglucomutases, allulose 6-phosphate epimerases, allulose 6-phosphate phosphatases, phosphoglucoisomerases selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), and optionally debranching enzymes to produce at least two cultured populations of cells expressing different enzymes; (b) lysing cells of the at least two cultured populations to produce at least two cell lysates; (c) combining the at least two cell lysates to produce a cell lysate mixture that comprises a cellodextrin phosphorylase, a phosphoglucomutase, a phosphoglucoisomerase, an allulose 6-phosphate epimerase, an allulose 6-phosphate phosphatase, and optionally a debranching enzyme; and (d) incubating the cell lysate mixture in the presence of cellulose/cellodextrin and inorganic phosphate to produce allulose.

Some aspects of the present disclosure provide a cell-free method for producing allulose, the method comprising (a) culturing at least two cell populations, wherein cells of each population are engineered to express at least one enzyme selected from the group consisting of α-glucan phosphorylases selected from the group consisting of AaGlgp (derived from Aquifex aeolicus), TzAgp (derived from Thermococcus zilligii), PtAgp (derived from Pseudothermotoga thermarum), Tm08495 (derived from Thermotoga maritima), TcGlgP (derived from Thermus caldophilus) and PfAgp (derived from Pyrococcus furiosus), phosphoglucomutases, phosphoglucoisomerases, allulose 6-phosphate epimerases, and allulose 6-phosphate phosphatases, and optionally debranching enzymes to produce at least two cultured populations of cells expressing different enzymes; (b) lysing cells of the at least two cultured populations to produce at least two cell lysates; (c) combining the cell lysates of step (b) to produce a cell lysate mixture that comprises an α-glucan phosphorylase, a phosphoglucomutase, a phosphoglucoisomerase, an allulose 6-phosphate epimerase, an allulose 6-phosphate phosphatase, and optionally a debranching enzyme; and (d) incubating the cell lysate mixture in the presence of a polysaccharide and inorganic phosphate to produce allulose.

Some embodiments of the present disclosure provide a cell-free method for producing allulose, the method comprising (a) culturing at least two cell populations, wherein cells of each population are engineered to express at least one enzyme selected from the group consisting of α-glucan phosphorylases, phosphoglucomutases, allulose 6-phosphate epimerases, allulose 6-phosphate phosphatases, a phosphoglucoisomerase selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), and optionally debranching enzymes to produce at least two cultured populations of cells expressing different enzymes; (b) lysing cells of the at least two cultured populations to produce at least two cell lysates; (c) combining the cell lysates of step (b) to produce a cell lysate mixture that comprises an α-glucan phosphorylase, a phosphoglucomutase, a phosphoglucoisomerase, an allulose 6-phosphate epimerase, an allulose 6-phosphate phosphatase, and optionally a debranching enzyme; and (d) incubating the cell lysate mixture in the presence of a polysaccharide and inorganic phosphate to produce allulose.

Some embodiments of the present disclosure provide a cell-free method for producing allulose, the method comprising (a) culturing at least two cell populations, wherein cells of each population are engineered to express at least one enzyme selected from the group consisting of cellodextrin phosphorylases, phosphoglucomutases, allulose 6-phosphate epimerases, allulose 6-phosphate phosphatases, a phosphoglucoisomerase selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), and optionally debranching enzymes to produce at least two cultured populations of cells expressing different enzymes; (b) lysing cells of the at least two cultured populations to produce at least two cell lysates; (c) combining the cell lysates of step (b) to produce a cell lysate mixture that comprises a cellodextrin phosphorylase, a phosphoglucomutase, a phosphoglucoisomerase, an allulose 6-phosphate epimerase, an allulose 6-phosphate phosphatase, and optionally a debranching enzyme; and (d) incubating the cell lysate mixture in the presence of cellulose/cellodextrin and inorganic phosphate to produce allulose.

Certain embodiments disclose a cell-free method for producing allulose, the method comprising (a) culturing at least two cell populations, wherein cells of each population are engineered to express at least one enzyme selected from the group consisting of α-glucan phosphorylases selected from the group consisting of AaGlgp (derived from Aquifex aeolicus), TzAgp (derived from Thermococcus zilligii), PtAgp (derived from Pseudothermotoga thermarum), Tm08495 (derived from Thermotoga maritima), TcGlgP (derived from Thermus caldophilus) and PfAgp (derived from Pyrococcus furiosus), phosphoglucomutases, phosphoglucoisomerases, allulose 6-phosphate epimerases, and allulose 6-phosphate phosphatases, and optionally debranching enzymes to produce at least two cultured populations of cells expressing different enzymes; (b) lysing cells of the at least two cultured populations to produce at least two cell lysates; (c) combining the at least two cell lysates to produce a cell lysate mixture; (d) adding to the cell lysate mixture at least one purified enzyme selected from the group consisting of α-glucan phosphorylases, phosphoglucomutases, phosphoglucoisomerases, allulose 6-phosphate epimerases, and allulose 6-phosphate phosphatases, and optionally debranching enzymes to produce a reaction mixture comprising an α-glucan phosphorylase, a phosphoglucomutase, a phosphoglucoisomerase, an allulose 6-phosphate epimerase, an allulose 6-phosphate phosphatase, and optionally a debranching enzyme; and (e) incubating the reaction mixture in the presence of a polysaccharide and inorganic phosphate to produce allulose.

In other embodiments, the present disclosure provides cell-free method for producing allulose, the method comprising (a) culturing at least two cell populations, wherein cells of each population are engineered to express at least one enzyme selected from the group consisting of α-glucan phosphorylases, phosphoglucomutases, allulose 6-phosphate epimerases, allulose 6-phosphate phosphatases, phosphoglucoisomerases selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), and optionally debranching enzymes to produce at least two cultured populations of cells expressing different enzymes; (b) lysing cells of the at least two cultured populations to produce at least two cell lysates; (c) combining the at least two cell lysates to produce a cell lysate mixture; (d) adding to the cell lysate mixture at least one purified enzyme selected from the group consisting of α-glucan phosphorylases, phosphoglucomutases, phosphoglucoisomerases, allulose 6-phosphate epimerases, allulose 6-phosphate phosphatases, and optionally debranching enzymes to produce a reaction mixture comprising an α-glucan phosphorylase, a phosphoglucomutase, a phosphoglucoisomerase, an allulose 6-phosphate epimerase, an allulose 6-phosphate phosphatase, and optionally a debranching enzyme; and (e) incubating the reaction mixture in the presence of a polysaccharide and inorganic phosphate to produce allulose.

In other embodiments, the present disclosure provides cell-free method for producing allulose, the method comprising (a) culturing at least two cell populations, wherein cells of each population are engineered to express at least one enzyme selected from the group consisting of cellodextrin phosphorylases, phosphoglucomutases, allulose 6-phosphate epimerases, allulose 6-phosphate phosphatases, phosphoglucoisomerases selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), and optionally debranching enzymes to produce at least two cultured populations of cells expressing different enzymes; (b) lysing cells of the at least two cultured populations to produce at least two cell lysates; (c) combining the at least two cell lysates to produce a cell lysate mixture; (d) adding to the cell lysate mixture at least one purified enzyme selected from the group consisting of cellodextrin phosphorylases, phosphoglucomutases, phosphoglucoisomerases, allulose 6-phosphate epimerases, allulose 6-phosphate phosphatases, and optionally debranching enzymes to produce a reaction mixture comprising a cellodextrin phosphorylase, a phosphoglucomutase, a phosphoglucoisomerase, an allulose 6-phosphate epimerase, an allulose 6-phosphate phosphatase, and optionally a debranching enzyme; and (e) incubating the reaction mixture in the presence of cellulose/cellodextrin and inorganic phosphate to produce allulose.

Some aspects of the present disclosure provide a cell-free method for producing allulose, the method comprising (a) culturing at least two cell populations, wherein cells of each population are engineered to express at least one enzyme selected from the group consisting of α-glucan phosphorylases selected from the group consisting of AaGlgp (derived from Aquifex aeolicus), TzAgp (derived from Thermococcus zilligii), PtAgp (derived from Pseudothermotoga thermarum), Tm08495 (derived from Thermotoga maritima), TcGlgP (derived from Thermus caldophilus) and PfAgp (derived from Pyrococcus furiosus), phosphoglucomutases, phosphoglucoisomerases, allulose 6-phosphate epimerases, and allulose 6-phosphate phosphatases, and optionally debranching enzymes to produce at least two cultured populations of cells expressing different enzymes; (b) lysing cells of the at least two cultured populations to produce at least two cell lysates; (c) combining the cell lysates of step (b) to produce a cell lysate mixture; (d) adding to the cell lysate mixture at least one purified enzyme selected from the group consisting of α-glucan phosphorylases, phosphoglucomutases, phosphoglucoisomerases, allulose 6-phosphate epimerases, allulose 6-phosphate phosphatases, and optionally debranching enzymes to produce a reaction mixture comprising an α-glucan phosphorylase, a phosphoglucomutase, a phosphoglucoisomerase, an allulose 6-phosphate epimerase, an allulose 6-phosphate phosphatase, and optionally a debranching enzyme; and (e) incubating the reaction mixture in the presence of a polysaccharide and inorganic phosphate to produce allulose.

Some embodiments of the present disclosure provide a cell-free method for producing allulose, the method comprising (a) culturing at least two cell populations, wherein cells of each population are engineered to express at least one enzyme selected from the group consisting of α-glucan phosphorylases, phosphoglucomutases, allulose 6-phosphate epimerases, and allulose 6-phosphate phosphatases, phosphoglucoisomerases selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), and optionally debranching enzymes to produce at least two cultured populations of cells expressing different enzymes; (b) lysing cells of the at least two cultured populations to produce at least two cell lysates; (c) combining the cell lysates of step (b) to produce a cell lysate mixture; (d) adding to the cell lysate mixture at least one purified enzyme selected from the group consisting of α-glucan phosphorylases, phosphoglucomutases, phosphoglucoisomerases, allulose 6-phosphate epimerases, allulose 6-phosphate phosphatases, and optionally debranching enzymes to produce a reaction mixture comprising an α-glucan phosphorylase, a phosphoglucomutase, a phosphoglucoisomerase, an allulose 6-phosphate epimerase, an allulose 6-phosphate phosphatase, and optionally a debranching enzyme; and (e) incubating the reaction mixture in the presence of a polysaccharide and inorganic phosphate to produce allulose.

Some embodiments of the present disclosure provide a cell-free method for producing allulose, the method comprising (a) culturing at least two cell populations, wherein cells of each population are engineered to express at least one enzyme selected from the group consisting of cellodextrin phosphorylases, phosphoglucomutases, allulose 6-phosphate epimerases, and allulose 6-phosphate phosphatases, phosphoglucoisomerases selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), and optionally debranching enzymes to produce at least two cultured populations of cells expressing different enzymes; (b) lysing cells of the at least two cultured populations to produce at least two cell lysates; (c) combining the cell lysates of step (b) to produce a cell lysate mixture; (d) adding to the cell lysate mixture at least one purified enzyme selected from the group consisting of cellodextrin phosphorylases, phosphoglucomutases, phosphoglucoisomerases, allulose 6-phosphate epimerases, allulose 6-phosphate phosphatases, and optionally debranching enzymes to produce a reaction mixture comprising a cellodextrin phosphorylase, a phosphoglucomutase, a phosphoglucoisomerase, an allulose 6-phosphate epimerase, an allulose 6-phosphate phosphatase, and optionally a debranching enzyme; and (e) incubating the reaction mixture in the presence of cellulose/cellodextrin and inorganic phosphate to produce allulose.

Some aspects of the present disclosure provide a cell-free method for producing allulose, the method comprising (a) culturing cells engineered to express a α-glucan phosphorylase selected from the group consisting of AaGlgp (derived from Aquifex aeolicus), TzAgp (derived from Thermococcus zilligii), PtAgp (derived from Pseudothermotoga thermarum), Tm08495 (derived from Thermotoga maritima), TcGlgP (derived from Thermus caldophilus) and PfAgp (derived from Pyrococcus furiosus), a phosphoglucomutase, a phosphoglucoisomerase, an allulose 6-phosphate epimerase, allulose 6-phosphate phosphatase, and optionally a debranching enzyme to produce cultured cells that express the enzymes; (b) lysing the cultured cells to produce a cell lysate; (c) heating the cell lysate to a temperature that inactivates native enzymatic activity but does not inactivate the enzymes of step (a) to produce a heat-inactivated lysate; and (d) incubating the heat-inactivated lysate in the presence of a polysaccharide and inorganic phosphate to produce allulose.

Some embodiments of the present disclosure provide a cell-free method for producing allulose, the method comprising (a) culturing cells engineered to express a α-glucan phosphorylase, a phosphoglucomutase, a allulose 6-phosphate epimerase, an allulose 6-phosphate phosphatase, optionally a debranching enzyme, and a phosphoglucoisomerase selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis) to produce cultured cells that express the enzymes; (b) lysing the cultured cells to produce a cell lysate; (c) heating the cell lysate to a temperature that inactivates native enzymatic activity but does not inactivate the enzymes of step (a) to produce a heat-inactivated lysate; and (d) incubating the heat-inactivated lysate in the presence of a polysaccharide and inorganic phosphate to produce allulose.

Some embodiments of the present disclosure provide a cell-free method for producing allulose, the method comprising (a) culturing cells engineered to express a cellodextrin phosphorylase, a phosphoglucomutase, a allulose 6-phosphate epimerase, an allulose 6-phosphate phosphatase, optionally a debranching enzyme, and a phosphoglucoisomerase selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis) to produce cultured cells that express the enzymes; (b) lysing the cultured cells to produce a cell lysate; (c) heating the cell lysate to a temperature that inactivates native enzymatic activity but does not inactivate the enzymes of step (a) to produce a heat-inactivated lysate; and (d) incubating the heat-inactivated lysate in the presence of cellulose/cellodextrin and inorganic phosphate to produce allulose.

Certain embodiments disclose a cell-free method for producing allulose, the method comprising (a) culturing at least two cell populations, wherein cells of each population are engineered to express at least one enzyme selected from the group consisting of α-glucan phosphorylases selected from the group consisting of AaGlgp (derived from Aquifex aeolicus), TzAgp (derived from Thermococcus zilligii), PtAgp (derived from Pseudothermotoga thermarum), Tm08495 (derived from Thermotoga maritima), TcGlgP (derived from Thermus caldophilus) and PfAgp (derived from Pyrococcus furiosus), phosphoglucomutases, phosphoglucoisomerases, allulose 6-phosphate epimerases, allulose 6-phosphate phosphatases, and optionally debranching enzymes to produce at least two cultured populations of cells expressing different enzymes; (b) lysing cells of the at least two cultured populations to produce at least two cell lysates; (c) combining the at least two cell lysates to produce a cell lysate mixture that comprises a α-glucan phosphorylase, a phosphoglucomutase, a phosphoglucoisomerase, a allulose 6-phosphate epimerase, an allulose 6-phosphate phosphatase, and optionally a debranching enzyme; (d) heating the cell lysate mixture to a temperature that inactivates native enzymatic activity but does not inactivate the enzymes of step (c) to produce a heat-inactivated lysate; and (e) incubating the reaction mixture in the presence of a polysaccharide and inorganic phosphate to produce allulose.

In other embodiments, the present disclosure provides a cell-free method for producing allulose, the method comprising (a) culturing at least two cell populations, wherein cells of each population are engineered to express at least one enzyme selected from the group consisting of α-glucan phosphorylases, phosphoglucomutases, allulose 6-phosphate epimerases, allulose 6-phosphate phosphatases, phosphoglucoisomerases selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), and optionally debranching enzymes to produce at least two cultured populations of cells expressing different enzymes; (b) lysing cells of the at least two cultured populations to produce at least two cell lysates; (c) combining the at least two cell lysates to produce a cell lysate mixture that comprises an α-glucan phosphorylase, a phosphoglucomutase, a phosphoglucoisomerase, a allulose 6-phosphate epimerase, an allulose 6-phosphate phosphatase, and optionally a debranching enzyme; (d) heating the cell lysate mixture to a temperature that inactivates native enzymatic activity but does not inactivate the enzymes of step (c) to produce a heat-inactivated lysate; and (e) incubating the reaction mixture in the presence of a polysaccharide and inorganic phosphate to produce allulose.

In other embodiments, the present disclosure provides a cell-free method for producing allulose, the method comprising (a) culturing at least two cell populations, wherein cells of each population are engineered to express at least one enzyme selected from the group consisting of cellodextrin phosphorylases, phosphoglucomutases, allulose 6-phosphate epimerases, allulose 6-phosphate phosphatases, phosphoglucoisomerases selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), and optionally debranching enzymes to produce at least two cultured populations of cells expressing different enzymes; (b) lysing cells of the at least two cultured populations to produce at least two cell lysates; (c) combining the at least two cell lysates to produce a cell lysate mixture that comprises a cellodextrin phosphorylase, a phosphoglucomutase, a phosphoglucoisomerase, a allulose 6-phosphate epimerase, an allulose 6-phosphate phosphatase, and optionally a debranching enzyme; (d) heating the cell lysate mixture to a temperature that inactivates native enzymatic activity but does not inactivate the enzymes of step (c) to produce a heat-inactivated lysate; and (e) incubating the reaction mixture in the presence of cellulose/cellodextrin and inorganic phosphate to produce allulose.

Some aspects of the present disclosure provide a cell-free method for producing allulose, the method comprising (a) culturing at least two cell populations, wherein cells of each population are engineered to express at least one enzyme selected from the group consisting of α-glucan phosphorylases selected from the group consisting of AaGlgp (derived from Aquifex aeolicus), TzAgp (derived from Thermococcus zilligii), PtAgp (derived from Pseudothermotoga thermarum), Tm08495 (derived from Thermotoga maritima), TcGlgP (derived from Thermus caldophilus) and PfAgp (derived from Pyrococcus furiosus), phosphoglucomutases, phosphoglucoisomerases, allulose 6-phosphate epimerases, allulose 6-phosphate phosphatases, and optionally debranching enzymes to produce at least two cultured populations of cells expressing different enzymes; (b) lysing cells of the at least two cultured populations to produce at least two cell lysates; (c) optionally heating one or more of the cell lysates of step (b) to a temperature that inactivates native enzymatic activity but does not inactivate the enzymes of step (a) to produce a heat-inactivated lysate; (d) combining the cell lysates of step (b) and (c) to produce a cell lysate mixture that comprises an α-glucan phosphorylase, a phosphoglucomutase, a phosphoglucoisomerase, an allulose 6-phosphate epimerase, an allulose 6-phosphate phosphatase, and optionally a debranching enzyme; and (e) incubating the cell lysate mixture in the presence of a polysaccharide and inorganic phosphate to produce allulose.

Some embodiments of the present disclosure provide a cell-free method for producing allulose, the method comprising (a) culturing at least two cell populations, wherein cells of each population are engineered to express at least one enzyme selected from the group consisting of α-glucan phosphorylases, phosphoglucomutases, allulose 6-phosphate epimerases, allulose 6-phosphate phosphatases, a phosphoglucoisomerase selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), and optionally debranching enzymes to produce at least two cultured populations of cells expressing different enzymes; (b) lysing cells of the at least two cultured populations to produce at least two cell lysates; (c) optionally heating one or more of the cell lysates of step (b) to a temperature that inactivates native enzymatic activity but does not inactivate the enzymes of step (a) to produce a heat-inactivated lysate; (d) combining the cell lysates of step (b) and (c) to produce a cell lysate mixture that comprises an α-glucan phosphorylase, a phosphoglucomutase, a phosphoglucoisomerase, an allulose 6-phosphate epimerase, an allulose 6-phosphate phosphatase, and optionally a debranching enzyme; and (e) incubating the cell lysate mixture in the presence of a polysaccharide and inorganic phosphate to produce allulose.

Some embodiments of the present disclosure provide a cell-free method for producing allulose, the method comprising (a) culturing at least two cell populations, wherein cells of each population are engineered to express at least one enzyme selected from the group consisting of cellodextrin phosphorylases, phosphoglucomutases, allulose 6-phosphate epimerases, allulose 6-phosphate phosphatases, a phosphoglucoisomerase selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), and optionally debranching enzymes to produce at least two cultured populations of cells expressing different enzymes; (b) lysing cells of the at least two cultured populations to produce at least two cell lysates; (c) optionally heating one or more of the cell lysates of step (b) to a temperature that inactivates native enzymatic activity but does not inactivate the enzymes of step (a) to produce a heat-inactivated lysate; (d) combining the cell lysates of step (b) and (c) to produce a cell lysate mixture that comprises a cellodextrin phosphorylase, a phosphoglucomutase, a phosphoglucoisomerase, an allulose 6-phosphate epimerase, an allulose 6-phosphate phosphatase, and optionally a debranching enzyme; and (e) incubating the cell lysate mixture in the presence of cellulose/cellodextrin and inorganic phosphate to produce allulose.

Certain embodiments disclose a cell-free method for producing allulose, the method comprising (a) culturing at least two cell populations, wherein cells of each population are engineered to express at least one enzyme selected from the group consisting of α-glucan phosphorylases selected from the group consisting of AaGlgp (derived from Aquifex aeolicus), TzAgp (derived from Thermococcus zilligii), PtAgp (derived from Pseudothermotoga thermarum), Tm08495 (derived from Thermotoga maritima), TcGlgP (derived from Thermus caldophilus) and PfAgp (derived from Pyrococcus furiosus), phosphoglucomutases, phosphoglucoisomerases, allulose 6-phosphate epimerases, allulose 6-phosphate phosphatases, and optionally debranching enzymes to produce at least two cultured populations of cells expressing different enzymes; (b) lysing cells of the at least two cultured populations to produce at least two cell lysates; (c) combining the at least two cell lysates to produce a cell lysate mixture; (d) heating the cell lysate mixture to a temperature that inactivates native enzymatic activity but does not inactivate the enzymes of step (a) to produce a heat-inactivated lysate; (e) adding to the heat-inactivated lysate at least one purified enzyme selected from the group consisting of α-glucan phosphorylases, phosphoglucomutases, phosphoglucoisomerases, allulose 6-phosphate epimerases, allulose 6-phosphate phosphatases, and optionally debranching enzymes to produce a reaction mixture comprising an α-glucan phosphorylase, a phosphoglucomutase, a phosphoglucoisomerase, an allulose 6-phosphate epimerase, an allulose 6-phosphate phosphatase, and optionally a debranching enzyme; and (f) incubating the reaction mixture in the presence of a polysaccharide and inorganic phosphate to produce allulose.

In other embodiments, the present disclosure provides cell-free method for producing allulose, the method comprising (a) culturing at least two cell populations, wherein cells of each population are engineered to express at least one enzyme selected from the group consisting of α-glucan phosphorylases, phosphoglucomutases, allulose 6-phosphate epimerases, allulose 6-phosphate phosphatases, phosphoglucoisomerases selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), and optionally debranching enzymes to produce at least two cultured populations of cells expressing different enzymes; (b) lysing cells of the at least two cultured populations to produce at least two cell lysates; (c) combining the at least two cell lysates to produce a cell lysate mixture; (d) heating the cell lysate mixture to a temperature that inactivates native enzymatic activity but does not inactivate the enzymes of step (a) to produce a heat-inactivated lysate; (e) adding to the heat-inactivated lysate at least one purified enzyme selected from the group consisting of α-glucan phosphorylases, phosphoglucomutases, phosphoglucoisomerases, allulose 6-phosphate epimerases, allulose 6-phosphate phosphatases, and optionally debranching enzymes to produce a reaction mixture comprising a α-glucan phosphorylase, a phosphoglucomutase, a phosphoglucoisomerase, an allulose 6-phosphate epimerase, an allulose 6-phosphate phosphatase, and optionally a debranching enzyme; and (f) incubating the reaction mixture in the presence of a polysaccharide and inorganic phosphate to produce allulose.

In other embodiments, the present disclosure provides cell-free method for producing allulose, the method comprising (a) culturing at least two cell populations, wherein cells of each population are engineered to express at least one enzyme selected from the group consisting of cellodextrin phosphorylases, phosphoglucomutases, allulose 6-phosphate epimerases, allulose 6-phosphate phosphatases, phosphoglucoisomerases selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), and optionally debranching enzymes to produce at least two cultured populations of cells expressing different enzymes; (b) lysing cells of the at least two cultured populations to produce at least two cell lysates; (c) combining the at least two cell lysates to produce a cell lysate mixture; (d) heating the cell lysate mixture to a temperature that inactivates native enzymatic activity but does not inactivate the enzymes of step (a) to produce a heat-inactivated lysate; (e) adding to the heat-inactivated lysate at least one purified enzyme selected from the group consisting of cellodextrin phosphorylases, phosphoglucomutases, phosphoglucoisomerases, allulose 6-phosphate epimerases, allulose 6-phosphate phosphatases, and optionally debranching enzymes to produce a reaction mixture comprising a cellodextrin phosphorylase, a phosphoglucomutase, a phosphoglucoisomerase, an allulose 6-phosphate epimerase, an allulose 6-phosphate phosphatase, and optionally a debranching enzyme; and (f) incubating the reaction mixture in the presence of cellulose/cellodextrin and inorganic phosphate to produce allulose.

Some aspects of the present disclosure provide a cell-free method for producing allulose, the method comprising (a) culturing at least two cell populations, wherein cells of each population are engineered to express at least one enzyme selected from the group consisting of α-glucan phosphorylases selected from the group consisting of AaGlgp (derived from Aquifex aeolicus), TzAgp (derived from Thermococcus zilligii), PtAgp (derived from Pseudothermotoga thermarum), Tm08495 (derived from Thermotoga maritima), TcGlgP (derived from Thermus caldophilus) and PfAgp (derived from Pyrococcus furiosus), phosphoglucomutases, phosphoglucoisomerases, allulose 6-phosphate epimerases, allulose 6-phosphate phosphatases, and optionally debranching enzymes to produce at least two cultured populations of cells expressing different enzymes; (b) lysing cells of the at least two cultured populations to produce at least two cell lysates; (c) optionally heating one or more of the cell lysates of step (b) to a temperature that inactivates native enzymatic activity but does not inactivate the enzymes of step (a) to produce a heat-inactivated lysate; (d) combining the cell lysates of step (b) and (c) to produce a cell lysate mixture; (e) adding to the cell lysate mixture at least one purified enzyme selected from the group consisting of α-glucan phosphorylases, phosphoglucomutases, phosphoglucoisomerases, allulose 6-phosphate epimerases, allulose 6-phosphate phosphatases, and optionally debranching enzymes to produce a reaction mixture comprising an α-glucan phosphorylase, a phosphoglucomutase, a phosphoglucoisomerase, an allulose 6-phosphate epimerase, an allulose 6-phosphate phosphatase, and optionally a debranching enzyme; and (f) incubating the reaction mixture in the presence of a polysaccharide and inorganic phosphate to produce allulose.

Some embodiments of the present disclosure provide a cell-free method for producing allulose, the method comprising (a) culturing at least two cell populations, wherein cells of each population are engineered to express at least one enzyme selected from the group consisting of α-glucan phosphorylases, phosphoglucomutases, allulose 6-phosphate epimerases, allulose 6-phosphate phosphatases, phosphoglucoisomerases selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), and optionally debranching enzymes to produce at least two cultured populations of cells expressing different enzymes; (b) lysing cells of the at least two cultured populations to produce at least two cell lysates; (c) optionally heating one or more of the cell lysates of step (b) to a temperature that inactivates native enzymatic activity but does not inactivate the enzymes of step (a) to produce a heat-inactivated lysate; (d) combining the cell lysates of step (b) and (c) to produce a cell lysate mixture; (e) adding to the cell lysate mixture at least one purified enzyme selected from the group consisting of α-glucan phosphorylases, phosphoglucomutases, phosphoglucoisomerases, allulose 6-phosphate epimerases, allulose 6-phosphate phosphatases, and optionally debranching enzymes to produce a reaction mixture comprising an α-glucan phosphorylase, a phosphoglucomutase, a phosphoglucoisomerase, an allulose 6-phosphate epimerase, an allulose 6-phosphate phosphatase, and optionally a debranching enzyme; and (f) incubating the reaction mixture in the presence of a polysaccharide and inorganic phosphate to produce allulose.

Some embodiments of the present disclosure provide a cell-free method for producing allulose, the method comprising (a) culturing at least two cell populations, wherein cells of each population are engineered to express at least one enzyme selected from the group consisting of cellodextrin phosphorylases, phosphoglucomutases, allulose 6-phosphate epimerases, allulose 6-phosphate phosphatases, phosphoglucoisomerases selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), and optionally debranching enzymes to produce at least two cultured populations of cells expressing different enzymes; (b) lysing cells of the at least two cultured populations to produce at least two cell lysates; (c) optionally heating one or more of the cell lysates of step (b) to a temperature that inactivates native enzymatic activity but does not inactivate the enzymes of step (a) to produce a heat-inactivated lysate; (d) combining the cell lysates of step (b) and (c) to produce a cell lysate mixture; (e) adding to the cell lysate mixture at least one purified enzyme selected from the group consisting of cellodextrin phosphorylases, phosphoglucomutases, phosphoglucoisomerases, allulose 6-phosphate epimerases, allulose 6-phosphate phosphatases, and optionally debranching enzymes to produce a reaction mixture comprising a cellodextrin phosphorylase, a phosphoglucomutase, a phosphoglucoisomerase, an allulose 6-phosphate epimerase, an allulose 6-phosphate phosphatase, and optionally a debranching enzyme; and (f) incubating the reaction mixture in the presence of cellulose/cellodextrin and inorganic phosphate to produce allulose.

Some aspects of the present disclosure provide a cell-free method for producing allulose, the method comprising (a) culturing cells engineered to express a thermostable α-glucan phosphorylase selected from the group consisting of AaGlgp (derived from Aquifex aeolicus), TzAgp (derived from Thermococcus zilligii), PtAgp (derived from Pseudothermotoga thermarum), Tm08495 (derived from Thermotoga maritima), TcGlgP (derived from Thermus caldophilus) and PfAgp (derived from Pyrococcus furiosus), a thermostable phosphoglucomutase, a thermostable phosphoglucoisomerase, a thermostable allulose 6-phosphate epimerase, a thermostable allulose 6-phosphate phosphatase, and optionally a thermostable debranching enzyme to produce cultured cells that express the thermostable enzymes; (b) lysing the cultured cells to produce a cell lysate; (c) heating the cell lysate to a temperature that inactivates native enzymatic activity but does not inactivate the thermostable enzymes of step (a) to produce a heat-inactivated lysate; and (d) incubating the heat-inactivated lysate in the presence of a polysaccharide and inorganic phosphate to produce allulose.

Some embodiments of the present disclosure provide a cell-free method for producing allulose, the method comprising (a) culturing cells engineered to express a thermostable α-glucan phosphorylase, a thermostable phosphoglucomutase, a thermostable allulose 6-phosphate epimerase, a thermostable allulose 6-phosphate phosphatase, a thermostable phosphoglucoisomerase selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), and optionally a thermostable debranching enzyme to produce cultured cells that express the thermostable enzymes; (b) lysing the cultured cells to produce a cell lysate; (c) heating the cell lysate to a temperature that inactivates native enzymatic activity but does not inactivate the thermostable enzymes of step (a) to produce a heat-inactivated lysate; and (d) incubating the heat-inactivated lysate in the presence of a polysaccharide and inorganic phosphate to produce allulose.

Some embodiments of the present disclosure provide a cell-free method for producing allulose, the method comprising (a) culturing cells engineered to express a thermostable cellodextrin phosphorylase, a thermostable phosphoglucomutase, a thermostable allulose 6-phosphate epimerase, a thermostable allulose 6-phosphate phosphatase, a thermostable phosphoglucoisomerase selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), and optionally a thermostable debranching enzyme to produce cultured cells that express the thermostable enzymes; (b) lysing the cultured cells to produce a cell lysate; (c) heating the cell lysate to a temperature that inactivates native enzymatic activity but does not inactivate the thermostable enzymes of step (a) to produce a heat-inactivated lysate; and (d) incubating the heat-inactivated lysate in the presence of cellulose/cellodextrin and inorganic phosphate to produce allulose.

Certain embodiments disclose a cell-free method for producing allulose, the method comprising (a) culturing at least two cell populations, wherein cells of each population are engineered to express at least one enzyme selected from the group consisting of thermostable α-glucan phosphorylases selected from the group consisting of AaGlgp (derived from Aquifex aeolicus), TzAgp (derived from Thermococcus zilligii), PtAgp (derived from Pseudothermotoga thermarum), Tm08495 (derived from Thermotoga maritima), TcGlgP (derived from Thermus caldophilus) and PfAgp (derived from Pyrococcus furiosus), thermostable phosphoglucomutases, thermostable phosphoglucoisomerases, thermostable allulose 6-phosphate epimerases, thermostable allulose 6-phosphate phosphatases, and optionally thermostable debranching enzymes to produce at least two cultured populations of cells expressing different enzymes; (b) lysing cells of the at least two cultured populations to produce at least two cell lysates; (c) combining the at least two cell lysates to produce a cell lysate mixture that comprises a thermostable α-glucan phosphorylase, a thermostable phosphoglucomutase, a thermostable phosphoglucoisomerase, a thermostable allulose 6-phosphate epimerase, a thermostable allulose 6-phosphate phosphatase, and optionally a thermostable debranching enzyme; (d) heating the cell lysate mixture to a temperature that inactivates native enzymatic activity but does not inactivate the thermostable enzymes of step (c) to produce a heat-inactivated lysate; and (e) incubating the reaction mixture in the presence of a polysaccharide and inorganic phosphate to produce allulose.

In other embodiments, the present disclosure provides a cell-free method for producing allulose, the method comprising (a) culturing at least two cell populations, wherein cells of each population are engineered to express at least one enzyme selected from the group consisting of thermostable α-glucan phosphorylases, thermostable phosphoglucomutases, thermostable allulose 6-phosphate epimerases, thermostable allulose 6-phosphate phosphatases, thermostable phosphoglucoisomerases selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), and optionally thermostable debranching enzymes to produce at least two cultured populations of cells expressing different enzymes; (b) lysing cells of the at least two cultured populations to produce at least two cell lysates; (c) combining the at least two cell lysates to produce a cell lysate mixture that comprises a thermostable α-glucan phosphorylase, a thermostable phosphoglucomutase, a thermostable phosphoglucoisomerase, a thermostable allulose 6-phosphate epimerase, a thermostable allulose 6-phosphate phosphatase, and optionally a thermostable debranching enzyme; (d) heating the cell lysate mixture to a temperature that inactivates native enzymatic activity but does not inactivate the thermostable enzymes of step (c) to produce a heat-inactivated lysate; and (e) incubating the reaction mixture in the presence of a polysaccharide and inorganic phosphate to produce allulose.

In other embodiments, the present disclosure provides a cell-free method for producing allulose, the method comprising (a) culturing at least two cell populations, wherein cells of each population are engineered to express at least one enzyme selected from the group consisting of thermostable cellodextrin phosphorylases, thermostable phosphoglucomutases, thermostable allulose 6-phosphate epimerases, thermostable allulose 6-phosphate phosphatases, thermostable phosphoglucoisomerases selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), and optionally thermostable debranching enzymes to produce at least two cultured populations of cells expressing different enzymes; (b) lysing cells of the at least two cultured populations to produce at least two cell lysates; (c) combining the at least two cell lysates to produce a cell lysate mixture that comprises a thermostable cellodextrin phosphorylase, a thermostable phosphoglucomutase, a thermostable phosphoglucoisomerase, a thermostable allulose 6-phosphate epimerase, a thermostable allulose 6-phosphate phosphatase, and optionally a thermostable debranching enzyme; (d) heating the cell lysate mixture to a temperature that inactivates native enzymatic activity but does not inactivate the thermostable enzymes of step (c) to produce a heat-inactivated lysate; and (e) incubating the reaction mixture in the presence of cellulose/cellodextrin and inorganic phosphate to produce allulose.

Some aspects of the present disclosure provide a cell-free method for producing allulose, the method comprising (a) culturing at least two cell populations, wherein cells of each population are engineered to express at least one enzyme selected from the group consisting of α-glucan phosphorylases selected from the group consisting of AaGlgp (derived from Aquifex aeolicus), TzAgp (derived from Thermococcus zilligii), PtAgp (derived from Pseudothermotoga thermarum), Tm08495 (derived from Thermotoga maritima), TcGlgP (derived from Thermus caldophilus) and PfAgp (derived from Pyrococcus furiosus), phosphoglucomutases, phosphoglucoisomerases, allulose 6-phosphate epimerases, allulose 6-phosphate phosphatases, and optionally debranching enzymes to produce at least two cultured populations of cells expressing different enzymes wherein at least one of the foregoing enzymes is thermostable; (b) lysing cells of the at least two cultured populations to produce at least two cell lysates; (c) optionally heating one or more of the cell lysates of step (b) to a temperature that inactivates native enzymatic activity but does not inactivate the thermostable enzymes of step (a) to produce a heat-inactivated lysate; (d) combining the cell lysates of step (b) and (c) to produce a cell lysate mixture that comprises an α-glucan phosphorylase, a phosphoglucomutase, a phosphoglucoisomerase, an allulose 6-phosphate epimerase, an allulose 6-phosphate phosphatase, and optionally a debranching enzyme, wherein at least one of the foregoing enzymes is thermostable; and (e) incubating the cell lysate mixture in the presence of a polysaccharide and inorganic phosphate to produce allulose.

Some embodiments of the present disclosure provide a cell-free method for producing allulose, the method comprising (a) culturing at least two cell populations, wherein cells of each population are engineered to express at least one enzyme selected from the group consisting of α-glucan phosphorylases, phosphoglucomutases, allulose 6-phosphate epimerases, allulose 6-phosphate phosphatases, a phosphoglucoisomerase selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), and optionally debranching enzymes to produce at least two cultured populations of cells expressing different enzymes wherein at least one of the foregoing enzymes is thermostable; (b) lysing cells of the at least two cultured populations to produce at least two cell lysates; (c) optionally heating one or more of the cell lysates of step (b) to a temperature that inactivates native enzymatic activity but does not inactivate the thermostable enzymes of step (a) to produce a heat-inactivated lysate; (d) combining the cell lysates of step (b) and (c) to produce a cell lysate mixture that comprises an α-glucan phosphorylase, a phosphoglucomutase, a phosphoglucoisomerase, an allulose 6-phosphate epimerase, an allulose 6-phosphate phosphatase, and optionally a debranching enzyme, wherein at least one of the foregoing enzymes is thermostable; and (e) incubating the cell lysate mixture in the presence of a polysaccharide and inorganic phosphate to produce allulose.

Some embodiments of the present disclosure provide a cell-free method for producing allulose, the method comprising (a) culturing at least two cell populations, wherein cells of each population are engineered to express at least one enzyme selected from the group consisting of cellodextrin phosphorylases, phosphoglucomutases, allulose 6-phosphate epimerases, allulose 6-phosphate phosphatases, a phosphoglucoisomerase selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), and optionally debranching enzymes to produce at least two cultured populations of cells expressing different enzymes wherein at least one of the foregoing enzymes is thermostable; (b) lysing cells of the at least two cultured populations to produce at least two cell lysates; (c) optionally heating one or more of the cell lysates of step (b) to a temperature that inactivates native enzymatic activity but does not inactivate the thermostable enzymes of step (a) to produce a heat-inactivated lysate; (d) combining the cell lysates of step (b) and (c) to produce a cell lysate mixture that comprises a cellodextrin phosphorylase, a phosphoglucomutase, a phosphoglucoisomerase, an allulose 6-phosphate epimerase, an allulose 6-phosphate phosphatase, and optionally a debranching enzyme, wherein at least one of the foregoing enzymes is thermostable; and (e) incubating the cell lysate mixture in the presence of cellulose/cellodextrin and inorganic phosphate to produce allulose.

Certain embodiments disclose a cell-free method for producing allulose, the method comprising (a) culturing at least two cell populations, wherein cells of each population are engineered to express at least one enzyme selected from the group consisting of thermostable α-glucan phosphorylases selected from the group consisting of AaGlgp (derived from Aquifex aeolicus), TzAgp (derived from Thermococcus zilligii), PtAgp (derived from Pseudothermotoga thermarum), Tm08495 (derived from Thermotoga maritima), TcGlgP (derived from Thermus caldophilus) and PfAgp (derived from Pyrococcus furiosus), thermostable phosphoglucomutases, thermostable phosphoglucoisomerases, thermostable allulose 6-phosphate epimerases, thermostable allulose 6-phosphate phosphatases, and optionally thermostable debranching enzymes to produce at least two cultured populations of cells expressing different enzymes; (b) lysing cells of the at least two cultured populations to produce at least two cell lysates; (c) combining the at least two cell lysates to produce a cell lysate mixture; (d) heating the cell lysate mixture to a temperature that inactivates native enzymatic activity but does not inactivate the thermostable enzymes of step (a) to produce a heat-inactivated lysate; (e) adding to the heat-inactivated lysate at least one purified enzyme selected from the group consisting of α-glucan phosphorylases, phosphoglucomutases, phosphoglucoisomerases, allulose 6-phosphate epimerases, allulose 6-phosphate phosphatases, and optionally debranching enzymes to produce a reaction mixture comprising an α-glucan phosphorylase, a phosphoglucomutase, a phosphoglucoisomerase, an allulose 6-phosphate epimerase, an allulose 6-phosphate phosphatase, and optionally a debranching enzyme; and (f) incubating the reaction mixture in the presence of a polysaccharide and inorganic phosphate to produce allulose.

In other embodiments, the present disclosure provides cell-free method for producing allulose, the method comprising (a) culturing at least two cell populations, wherein cells of each population are engineered to express at least one enzyme selected from the group consisting of thermostable α-glucan phosphorylases, thermostable phosphoglucomutases, thermostable allulose 6-phosphate epimerases, thermostable allulose 6-phosphate phosphatases, thermostable phosphoglucoisomerases selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), and optionally thermostable debranching enzymes to produce at least two cultured populations of cells expressing different enzymes; (b) lysing cells of the at least two cultured populations to produce at least two cell lysates; (c) combining the at least two cell lysates to produce a cell lysate mixture; (d) heating the cell lysate mixture to a temperature that inactivates native enzymatic activity but does not inactivate the thermostable enzymes of step (a) to produce a heat-inactivated lysate; (e) adding to the heat-inactivated lysate at least one purified enzyme selected from the group consisting of α-glucan phosphorylases, phosphoglucomutases, phosphoglucoisomerases, allulose 6-phosphate epimerases, allulose 6-phosphate phosphatases, and optionally debranching enzymes to produce a reaction mixture comprising a α-glucan phosphorylase, a phosphoglucomutase, a phosphoglucoisomerase, an allulose 6-phosphate epimerase, an allulose 6-phosphate phosphatase, and optionally a debranching enzyme; and (f) incubating the reaction mixture in the presence of a polysaccharide and inorganic phosphate to produce allulose.

In other embodiments, the present disclosure provides cell-free method for producing allulose, the method comprising (a) culturing at least two cell populations, wherein cells of each population are engineered to express at least one enzyme selected from the group consisting of thermostable cellodextrin phosphorylases, thermostable phosphoglucomutases, thermostable allulose 6-phosphate epimerases, thermostable allulose 6-phosphate phosphatases, thermostable phosphoglucoisomerases selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), and optionally thermostable debranching enzymes to produce at least two cultured populations of cells expressing different enzymes; (b) lysing cells of the at least two cultured populations to produce at least two cell lysates; (c) combining the at least two cell lysates to produce a cell lysate mixture; (d) heating the cell lysate mixture to a temperature that inactivates native enzymatic activity but does not inactivate the thermostable enzymes of step (a) to produce a heat-inactivated lysate; (e) adding to the heat-inactivated lysate at least one purified enzyme selected from the group consisting of cellodextrin phosphorylases, phosphoglucomutases, phosphoglucoisomerases, allulose 6-phosphate epimerases, allulose 6-phosphate phosphatases, and optionally debranching enzymes to produce a reaction mixture comprising a cellodextrin phosphorylase, a phosphoglucomutase, a phosphoglucoisomerase, an allulose 6-phosphate epimerase, an allulose 6-phosphate phosphatase, and optionally a debranching enzyme; and (f) incubating the reaction mixture in the presence of cellulose/cellodextrin and inorganic phosphate to produce allulose.

Some aspects of the present disclosure provide a cell-free method for producing allulose, the method comprising (a) culturing at least two cell populations, wherein cells of each population are engineered to express at least one enzyme selected from the group consisting of α-glucan phosphorylases selected from the group consisting of AaGlgp (derived from Aquifex aeolicus), TzAgp (derived from Thermococcus zilligii), PtAgp (derived from Pseudothermotoga thermarum), Tm08495 (derived from Thermotoga maritima), TcGlgP (derived from Thermus caldophilus) and PfAgp (derived from Pyrococcus furiosus), phosphoglucomutases, phosphoglucoisomerases, allulose 6-phosphate epimerases, allulose 6-phosphate phosphatases, and optionally debranching enzymes to produce at least two cultured populations of cells expressing different enzymes, wherein at least one of the foregoing enzymes is thermostable; (b) lysing cells of the at least two cultured populations to produce at least two cell lysates; (c) optionally heating one or more of the cell lysates of step (b) to a temperature that inactivates native enzymatic activity but does not inactivate the thermostable enzymes of step (a) to produce a heat-inactivated lysate; (d) combining the cell lysates of step (b) and (c) to produce a cell lysate mixture; (e) adding to the cell lysate mixture at least one purified enzyme selected from the group consisting of α-glucan phosphorylases, phosphoglucomutases, phosphoglucoisomerases, allulose 6-phosphate epimerases, allulose 6-phosphate phosphatases, and optionally debranching enzymes to produce a reaction mixture comprising an α-glucan phosphorylase, a phosphoglucomutase, a phosphoglucoisomerase, an allulose 6-phosphate epimerase, an allulose 6-phosphate phosphatase, and optionally a debranching enzyme; and (f) incubating the reaction mixture in the presence of a polysaccharide and inorganic phosphate to produce allulose.

Some embodiments of the present disclosure provide a cell-free method for producing allulose, the method comprising (a) culturing at least two cell populations, wherein cells of each population are engineered to express at least one enzyme selected from the group consisting of α-glucan phosphorylases, phosphoglucomutases, allulose 6-phosphate epimerases, allulose 6-phosphate phosphatases, phosphoglucoisomerases selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), and optionally debranching enzymes to produce at least two cultured populations of cells expressing different enzymes, wherein at least one of the foregoing enzymes is thermostable; (b) lysing cells of the at least two cultured populations to produce at least two cell lysates; (c) optionally heating one or more of the cell lysates of step (b) to a temperature that inactivates native enzymatic activity but does not inactivate the thermostable enzymes of step (a) to produce a heat-inactivated lysate; (d) combining the cell lysates of step (b) and (c) to produce a cell lysate mixture; (e) adding to the cell lysate mixture at least one purified enzyme selected from the group consisting of α-glucan phosphorylases, phosphoglucomutases, phosphoglucoisomerases, allulose 6-phosphate epimerases, allulose 6-phosphate phosphatases, and optionally debranching enzymes to produce a reaction mixture comprising an α-glucan phosphorylase, a phosphoglucomutase, a phosphoglucoisomerase, an allulose 6-phosphate epimerase, an allulose 6-phosphate phosphatase, and optionally a debranching enzyme; and (f) incubating the reaction mixture in the presence of a polysaccharide and inorganic phosphate to produce allulose.

Some embodiments of the present disclosure provide a cell-free method for producing allulose, the method comprising (a) culturing at least two cell populations, wherein cells of each population are engineered to express at least one enzyme selected from the group consisting of cellodextrin phosphorylases, phosphoglucomutases, allulose 6-phosphate epimerases, allulose 6-phosphate phosphatases, phosphoglucoisomerases selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), and optionally debranching enzymes to produce at least two cultured populations of cells expressing different enzymes, wherein at least one of the foregoing enzymes is thermostable; (b) lysing cells of the at least two cultured populations to produce at least two cell lysates; (c) optionally heating one or more of the cell lysates of step (b) to a temperature that inactivates native enzymatic activity but does not inactivate the thermostable enzymes of step (a) to produce a heat-inactivated lysate; (d) combining the cell lysates of step (b) and (c) to produce a cell lysate mixture; (e) adding to the cell lysate mixture at least one purified enzyme selected from the group consisting of cellodextrin phosphorylases, phosphoglucomutases, phosphoglucoisomerases, allulose 6-phosphate epimerases, allulose 6-phosphate phosphatases, and optionally debranching enzymes to produce a reaction mixture comprising a cellodextrin phosphorylase, a phosphoglucomutase, a phosphoglucoisomerase, an allulose 6-phosphate epimerase, an allulose 6-phosphate phosphatase, and optionally a debranching enzyme; and (f) incubating the reaction mixture in the presence of cellulose/cellodextrin and inorganic phosphate to produce allulose.

Some aspects of the present disclosure provide a cell-free method for producing allulose, the method comprising (a) culturing cells engineered to express a debranching enzyme (b) lysing the cultured cells of step (a) to produce a cell lysate; (c) in a second reaction, culturing at least two cell populations, wherein cells of each population are engineered to express at least one enzyme selected from the group consisting of α-glucan phosphorylases selected from the group consisting of AaGlgp (derived from Aquifex aeolicus), TzAgp (derived from Thermococcus zilligii), PtAgp (derived from Pseudothermotoga thermarum), Tm08495 (derived from Thermotoga maritima), TcGlgP (derived from Thermus caldophilus) and PfAgp (derived from Pyrococcus furiosus), phosphoglucomutases, phosphoglucoisomerases selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera), allulose 6-phosphate epimerases, and allulose 6-phosphate phosphatases to produce cultured cells that have expressed the enzymes; (d) lysing cells of the at least two cultured populations in step (c) to produce at least two cell lysates; (e) combining the cell lysates from step (b) and step (c) to create a cell lysate mixture; (f) incubating the cell lysate mixture in the presence of a polysaccharide and inorganic phosphate to produce allulose.

Some aspects of the present disclosure provide a cell-free method for producing allulose, the method comprising (a) culturing cells engineered to express a debranching enzyme (b) lysing the cultured cells of step (a) to produce a cell lysate; (c) in a second reaction, culturing at least two cell populations, wherein cells of each population are engineered to express at least one enzyme selected from the group consisting of cellodextrin phosphorylases, phosphoglucomutases, phosphoglucoisomerases selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera), allulose 6-phosphate epimerases, and allulose 6-phosphate phosphatases to produce cultured cells that have expressed the enzymes; (d) lysing cells of the at least two cultured populations in step (c) to produce at least two cell lysates; (e) combining the cell lysates from step (b) and step (c) to create a cell lysate mixture; (f) incubating the cell lysate mixture in the presence of cellulose/cellodextrin and inorganic phosphate to produce allulose.

Some embodiments of the present disclosure provide a cell-free method for producing allulose, the method comprising (a) culturing at least two cell populations, wherein cells of each population are engineered to express at least one debranching enzyme, α-glucan phosphorylase selected from the group consisting of AaGlgp (derived from Aquifex aeolicus), TzAgp (derived from Thermococcus zilligii), PtAgp (derived from Pseudothermotoga thermarum), Tm08495 (derived from Thermotoga maritima), TcGlgP (derived from Thermus caldophilus) and PfAgp (derived from Pyrococcus furiosus), phosphoglucomutase, phosphoglucoisomerase selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera), phosphoglucomutase, allulose 6-phosphate epimerase, allulose 6-phosphate phosphatase, phosphoglucoisomerase selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), to produce at least two cultured populations of cells expressing different enzymes, wherein at least one of the foregoing enzymes is thermostable; (b) lysing cells of the at least two cultured populations to produce at least two cell lysates; (c) optionally heating one or more of the cell lysates of step (b) to a temperature that inactivates native enzymatic activity but does not inactivate the thermostable enzymes of step (a) to produce a heat-inactivated lysate; (d) combining the cell lysates of step (b) and (c) to produce a cell lysate mixture; and (e) incubating the reaction mixture in the presence of a polysaccharide and inorganic phosphate to produce allulose.

Some embodiments of the present disclosure provide a cell-free method for producing allulose, the method comprising (a) culturing at least two cell populations, wherein cells of each population are engineered to express at least one debranching enzyme, cellodextrin phosphorylase, phosphoglucomutase, phosphoglucoisomerase selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera), phosphoglucomutase, allulose 6-phosphate epimerase, allulose 6-phosphate phosphatase, phosphoglucoisomerase selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis), to produce at least two cultured populations of cells expressing different enzymes, wherein at least one of the foregoing enzymes is thermostable; (b) lysing cells of the at least two cultured populations to produce at least two cell lysates; (c) optionally heating one or more of the cell lysates of step (b) to a temperature that inactivates native enzymatic activity but does not inactivate the thermostable enzymes of step (a) to produce a heat-inactivated lysate; (d) combining the cell lysates of step (b) and (c) to produce a cell lysate mixture; and (e) incubating the reaction mixture in the presence of cellulose/cellodextrin and inorganic phosphate to produce allulose.

Any of the enzymes described herein may be provided in a kit. In some embodiments, the kit comprises an enzyme provided herein. In some embodiments, the kit comprises cells for expressing an enzyme as described here. In some embodiments, the kit comprises cells for expressing at least one of a debranching enzyme, an α-glucan phosphorylase, a phosphoglucomutase, a phosphoglucoisomerase, an allulose 6-phosphate epimerase, and an allulose 6-phosphate phosphatase. In some embodiments, the kit comprises cells for expressing at least one Tm08495, AaGlgP, TcGlgP, PtAgp, TzAgp, PfAgp, CtPgi, TtPgi, MjPgi, PfPgi, Ap0768, and Cl1150. In some embodiments, the kit comprises a nucleic acid vector for expressing an enzyme as described herein. In some embodiments, the kit comprises a nucleic acid vector for expressing at least one of a debranching enzyme, an α-glucan phosphorylase, a phosphoglucomutase, a phosphoglucoisomerase, an allulose 6-phosphate epimerase, and an allulose 6-phosphate phosphatase. In some embodiments, the kit comprises a nucleic acid vector for expressing at least one of Tm08495, AaGlgP, TcGlgP, PtAgp, TzAgp, PfAgp, CtPgi, TtPgi, MjPgi, PfPgi, Ap0768, and Cl1150. In some embodiments, the kit comprises cells for expressing at least one of a debranching enzyme, a cellodextrin phosphorylase, a phosphoglucomutase, a phosphoglucoisomerase, an allulose 6-phosphate epimerase, and an allulose 6-phosphate phosphatase. In some embodiments, the kit comprises a nucleic acid vector for expressing at least one of a debranching enzyme, a cellodextrin phosphorylase, a phosphoglucomutase, a phosphoglucoisomerase, an allulose 6-phosphate epimerase, and an allulose 6-phosphate phosphatase.

In some embodiments, the kit further comprises at least one reagent for performing a method described herein including, but not limited to, methods of producing allulose, methods of converting a polysaccharide to allulose, methods of converting maltodextrin to allulose, methods of preparing any one of glucose 1-phosphate, glucose 6-phosphate, fructose 6-phosphate, and/or allulose 6-phosphate. In some embodiments, the at least one reagent includes, but is not limited to a polysaccharide, maltodextrin, glucose 1-phosphate, glucose 6-phosphate, fructose 6-phosphate, allulose 6-phosphate, an inorganic phosphate, and a cofactor. In some embodiments, the kit further comprises at least one reagent for performing a method described herein including, but not limited to, methods of producing allulose, methods of converting cellulose/cellodextrin to allulose, methods of converting maltodextrin to allulose, methods of preparing any one of glucose 1-phosphate, glucose 6-phosphate, fructose 6-phosphate, and/or allulose 6-phosphate. In some embodiments, the at least one reagent includes, but is not limited to cellulose, cellodextrin, maltodextrin, glucose 1-phosphate, glucose 6-phosphate, fructose 6-phosphate, allulose 6-phosphate, an inorganic phosphate, and a cofactor.

In some embodiments, the kit comprises an inorganic phosphate and cells for expressing an α-glucan phosphorylase, a phosphoglucomutase, a phosphoglucoisomerase, an allulose 6-phosphate epimerase, and an allulose 6-phosphate phosphatase for performing the method described herein of converting a polysaccharide to allulose. In some embodiments, the kit comprises inorganic phosphate and cells for expressing a debranching enzyme, an α-glucan phosphorylase, a phosphoglucomutase, a phosphoglucoisomerase, an allulose 6-phosphate epimerase, and an allulose 6-phosphate phosphatase for performing the method described herein of converting a polysaccharide to allulose. In some embodiments, the kit comprises an inorganic phosphate and cells for expressing a cellodextrin phosphorylase, a phosphoglucomutase, a phosphoglucoisomerase, an allulose 6-phosphate epimerase, and an allulose 6-phosphate phosphatase for performing the method described herein of converting cellulose/cellodextrin to allulose. In some embodiments, the kit comprises inorganic phosphate and cells for expressing a debranching enzyme, a cellodextrin phosphorylase, a phosphoglucomutase, a phosphoglucoisomerase, an allulose 6-phosphate epimerase, and an allulose 6-phosphate phosphatase for performing the method described herein of converting cellulose/cellodextrin to allulose.

Also provided herein are engineered cells, cell lysates, reaction mixtures, and kits comprising enzymes, such as thermostable enzymes, used for the production of allulose. Related allulose production pathways to which the enzymes herein may be applied appear in International Publication No. WO 2018/129275 A1, published Jul. 12, 2018, incorporated herein by reference.

The details of one or more embodiments of the invention are set forth herein. Other features, objects, and advantages of the invention will be apparent from the Detailed Description, the Examples, and the Claims.

### BRIEF DESCRIPTION OF THE DRAWINGS

FIG. 1 is a schematic of an enzymatic pathway for the conversion of a polysaccharide to allulose.

FIG. 2 is a schematic of an enzymatic pathway for the conversion of cellulose to allulose.

FIG. 3 demonstrates the production of of allulose (solid), fructose (dashed), and glucose (dotted) from maltodextrin by end-to-end allulose pathway, using PtAgP as the α-glucan phosphorylase.

FIG. 4 demonstrates the production of allulose (solid), fructose (dashed), and glucose (dotted) from maltodextrin by end-to-end allulose pathway, using TzAgP as the α-glucan phosphorylase.

FIG. 5 demonstrates the Production of allulose (solid), fructose (dashed), and glucose (dotted) from maltodextrin by end-to-end allulose pathway, using PfPgi as the phosphoglucoisomerase.

FIG. 6 demonstrates the production of allulose (solid), fructose (dashed), and glucose (dotted) from maltodextrin by end-to-end allulose pathway, using Ap0768 as the phosphoglucoisomerase.

FIG. 7 Production of allulose (solid), fructose (dashed), and glucose (dotted) from maltodextrin by end-to-end allulose pathway, using Cl1150 as the phosphoglucoisomerase.

### DETAILED DESCRIPTION

Described herein are enzymatic pathways used for the conversion of a polysaccharide to allulose. The enzymatic pathway utilizes at least one α-glucan phosphorylase, at least one phosphoglucomutase, at least one phosphoglucoisomerase, at least one allulose 6-phosphate 3-epimerase, and at least one allulose 6-phosphate phosphatase, and optionally, a debranching enzyme, for example, either a pullulanase or isoamylase. In some embodiments, the enzymes or a subset of the enzymes are thermostable. These thermostable enzymes can withstand an optional heating step of the sugar production process that inactivates deleterious activities contained within cellular lysates and this heat inactivation step decreases the chances of microbial contamination negatively impacting the production of allulose.

The present disclosure provides, in some embodiments, highly-efficient and cost-effective methods, compositions, and systems for producing allulose. These methods, compositions and systems for producing allulose are highly-efficient and cost-effective due to favorable thermodynamics. The reaction thermodynamics favor the product in that the final enzymatic step is irreversible, allowing for high yields of allulose. The ability to directly transform a polysaccharide to allulose in a cell-free system as described herein reduces costs by reducing downstream processing and non-converted substrate.

Non-limiting examples of allulose production pathways and pathway enzymes are provided in Table 1 below.

TABLE 1
Summary of Exemplary Pathway Enzymes

| Pathway | Step | Enzymes used | Transformation |
|---|---|---|---|
| 1 | 1 | α-glucan phosphorylase | polysaccharide to glucose 1-phosphate |
| | 2 | phosphoglucomutase | glucose 1-phosphate to glucose 6- |
| | | | phosphate |
| | 3 | phosphoglucoisomerase | glucose 6-phosphate to fructose 6- |
| | | | phosphate |
| | 4 | allulose 6-phosphate epimerase | fructose 6-phosphate to allulose 6- |
| | | | phosphate |
| | 5 | allulose 6-phosphate phosphatase | allulose 6-phosphate to allulose |
| 2 | D1 | pullulanase | debranching |
| | 1 | α-glucan phosphorylase | polysaccharide to glucose 1-phosphate |
| | 2 | phosphoglucomutase | glucose 1-phosphate to glucose 6- |
| | | | phosphate |
| | 3 | phosphoglucoisomerase | glucose 6-phosphate to fructose 6- |
| | | | phosphate |
| | 4 | allulose 6-phosphate epimerase | fructose 6-phosphate to allulose 6- |
| | | | phosphate |
| | 5 | allulose 6-phosphate phosphatase | allulose 6-phosphate to allulose |
| 3 | D2 | isoamylase | debranching |
| | 1 | α-glucan phosphorylase | polysaccharide to glucose 1-phosphate |
| | 2 | phosphoglucomutase | glucose 1-phosphate to glucose 6- |
| | | | phosphate |
| | 3 | phosphoglucoisomerase | glucose 6-phosphate to fructose 6- |
| | | | phosphate |
| | 4 | allulose 6-phosphate epimerase | fructose 6-phosphate to allulose 6- |
| | | | phosphate |
| | 5 | allulose 6-phosphate phosphatase | allulose 6-phosphate to allulose |
| 4 | D1 | pullulanase | debranching |
| | D2 | isoamylase | debranching |
| | 1 | α-glucan phosphorylase | polysaccharide to glucose 1-phosphate |
| | 2 | phosphoglucomutase | glucose 1-phosphate to glucose 6- |
| | | | phosphate |
| | 3 | phosphoglucoisomerase | glucose 6-phosphate to fructose 6- |
| | | | phosphate |
| | 4 | allulose 6-phosphate epimerase | fructose 6-phosphate to allulose 6- |
| | | | phosphate |
| | 5 | allulose 6-phosphate phosphatase | allulose 6-phosphate to allulose |
| 5 | 1 | cellodextrin phosphorylase | cellulose/cellodextrin to glucose 1- |
| | | | phosphate |
| | 2 | phosphoglucomutase | glucose 1-phosphate to glucose 6- |
| | | | phosphate |
| | 3 | phosphoglucoisomerase | glucose 6-phosphate to fructose 6- |
| | | | phosphate |
| | 4 | allulose 6-phosphate epimerase | fructose 6-phosphate to allulose 6- |
| | | | phosphate |
| | 5 | allulose 6-phosphate phosphatase | allulose 6-phosphate to allulose |
| 6 | D1 | pullulanase | debranching |
| | 1 | cellodextrin phosphorylase | cellulose/cellodextrin to glucose 1- |
| | | | phosphate |
| | 2 | phosphoglucomutase | glucose 1-phosphate to glucose 6- |
| | | | phosphate |
| | 3 | phosphoglucoisomerase | glucose 6-phosphate to fructose 6- |
| | | | phosphate |
| | 4 | allulose 6-phosphate epimerase | fructose 6-phosphate to allulose 6- |
| | | | phosphate |
| | 5 | allulose 6-phosphate phosphatase | allulose 6-phosphate to allulose |
| 7 | D2 | isoamylase | debranching |
| | 1 | cellodextrin phosphorylase | cellulose/cellodextrin to glucose 1- |
| | | | phosphate |
| | 2 | phosphoglucomutase | glucose 1-phosphate to glucose 6- |
| | | | phosphate |
| | 3 | phosphoglucoisomerase | glucose 6-phosphate to fructose 6- |
| | | | phosphate |
| | 4 | allulose 6-phosphate epimerase | fructose 6-phosphate to allulose 6- |
| | | | phosphate |
| | 5 | allulose 6-phosphate phosphatase | allulose 6-phosphate to allulose |
| 8 | D1 | pullulanase | debranching |
| | D2 | isoamylase | debranching |
| | 1 | cellodextrin phosphorylase | cellulose/cellodextrin to glucose 1- |
| | | | phosphate |
| | 2 | phosphoglucomutase | glucose 1-phosphate to glucose 6- |
| | | | phosphate |
| | 3 | phosphoglucoisomerase | glucose 6-phosphate to fructose 6- |
| | | | phosphate |
| | 4 | allulose 6-phosphate epimerase | fructose 6-phosphate to allulose 6- |
| | | | phosphate |
| | 5 | allulose 6-phosphate phosphatase | allulose 6-phosphate to allulose |

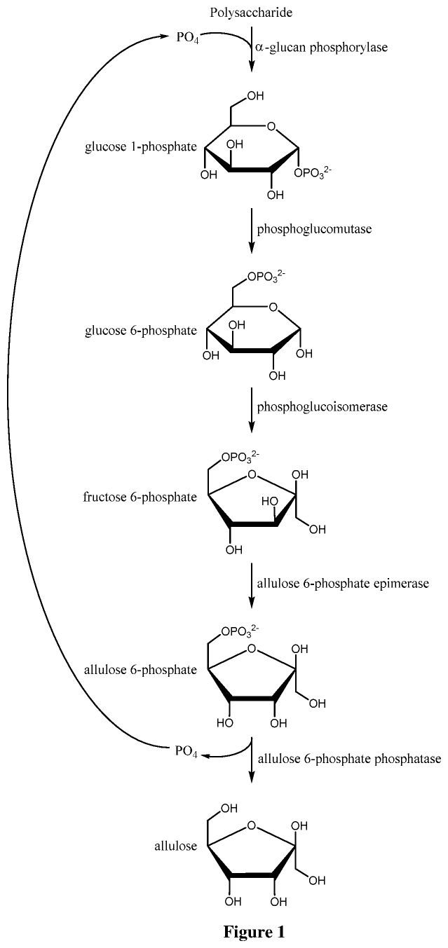

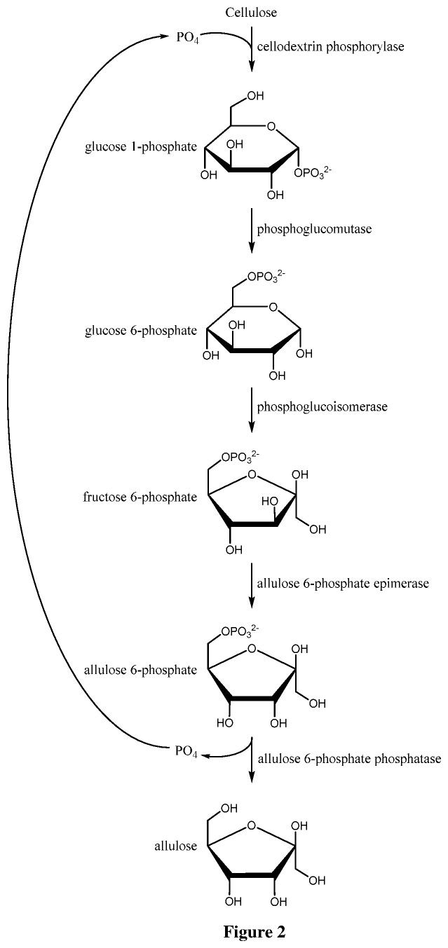

It should be understood that the allulose production pathways presented herein and in Table 1 are multistep processes. In some embodiments Pathway 1, 2, 3, and/or 4 are used to convert a polysaccharide to allulose (Table 1 and FIG. 1). In some embodiments Pathway 5, 6, 7, and/or 8 are used to convert cellulose/cellodextrin to allulose (Table 1 and FIG. 2). In other embodiments, an individual step may be used to perform the corresponding transformation (e.g., step 2 may be used to convert glucose 1-phosphate to glucose 6-phosphate). In some embodiments, a combination of steps less than the total pathway are used (e.g., combining steps 2, 3, and 4 to convert glucose 1-phosphate to allulose 6-phosphate).

### Allulose Production

Some aspects of the present disclosure provide methods, compositions, and systems for producing allulose. These methods, in some embodiments, include culturing cells engineered to express at least one pullulanase or isoamylase, at least one α-glucan phosphorylase, at least one phosphoglucomutase, at least one phosphoglucoisomerase, at least one allulose 6-phosphate epimerase, at least one allulose 6-phosphate phosphatase, or a combination of at least two (e.g., at least three, or at least four) of the foregoing enzymes. These methods, in some embodiments, include culturing cells engineered to express at least one pullulanase or isoamylase, at least one cellodextrin phosphorylase, at least one phosphoglucomutase, at least one phosphoglucoisomerase, at least one allulose 6-phosphate epimerase, at least one allulose 6-phosphate phosphatase, or a combination of at least two (e.g., at least three, or at least four) of the foregoing enzymes.

Enzymes of the allulose production pathways as provided herein are typically heterologous to the host cell, although some of the enzymes may be endogenous (native) to the host cell. Thus, in some embodiments, at least one enzyme (e.g., thermostable enzyme) used to convert a polysaccharide to allulose is heterologous to the host cell. In some embodiments, at least two, at least three, or at least four enzymes are heterologous to the host cell. In some embodiments, at least one enzyme is endogenous (native) to the host cell. In some embodiments, at least two, at least three, or at least four enzymes are endogenous to the host cell.

The host cells may be prokaryotic cells, such as bacterial cells (e.g., Escherichia coli cells), or eukaryotic cells, such as yeast cells or plant cells. Other cell types are described below.

In some embodiments, at least one of the enzymes used to convert a polysaccharide to allulose is a thermostable enzyme. In some embodiments, at least two (e.g., at least three or at least four) of the enzymes are thermostable enzymes. In some embodiments, all of the enzymes are thermostable enzymes. Thus, in some embodiments, the methods include culturing cells engineered to express at least one thermostable α-glucan phosphorylase, at least one thermostable phosphoglucomutase, at least one thermostable phosphoglucoisomerase, at least one thermostable allulose 6-phosphate epimerase, at least one thermostable allulose 6-phosphate phosphatase, optionally at least one debranching enzyme, or a combination of at least two or more of the foregoing thermostable enzymes.

In some embodiments, at least one of the enzymes used to convert cellulose/cellodextrin to allulose is a thermostable enzyme. In some embodiments, at least two (e.g., at least three or at least four) of the enzymes are thermostable enzymes. In some embodiments, all of the enzymes are thermostable enzymes. Thus, in some embodiments, the methods include culturing cells engineered to express at least one thermostable cellodextrin phosphorylase, at least one thermostable phosphoglucomutase, at least one thermostable phosphoglucoisomerase, at least one thermostable allulose 6-phosphate epimerase, at least one thermostable allulose 6-phosphate phosphatase, optionally at least one debranching enzyme, or a combination of at least two or more of the foregoing thermostable enzymes.

In some embodiments, the methods of producing allulose include lysing (e.g., thermal, osmotic, mechanical, chemical, or enzymatic lysis) the cultured cells to produce at least one (e.g., at least two, at least three, or at least four) cell lysate. It should be understood that multiple cell lysates (and thus multiple cell populations, e.g., from the same organism (e.g., bacteria) or from different organisms (e.g., bacteria, yeast, and/or plant cells)) may be used in the production of allulose as provided herein. For example, one cell population may be engineered to express one or more enzymes(s) of the allulose production pathway, while another cell population (or several other cell populations) may be engineered to express another (at least one other) enzyme of the allulose production pathway. Thus, in some embodiments, the methods comprise culturing at least one population of cells engineered to express at least one α-glucan phosphorylase, culturing at least one cell population engineered to express at least one phosphoglucomutase, culturing at least one cell population engineered to express at least one phosphoglucoisomerase, culturing at least one cell population engineered to express at least one allulose 6-phosphate epimerase, culturing at least one cell population engineered to express at least one allulose 6-phosphate phosphatase, and/or culturing at least one cell population engineered to express at least one debranching enzyme. Following lysis of the cells, the cell lysates are combined such that the enzymes are present in a single cell lysate/reaction mixture. Thus, in some embodiments, the methods comprise culturing at least one population of cells engineered to express at least one cellodextrin phosphorylase, culturing at least one cell population engineered to express at least one phosphoglucomutase, culturing at least one cell population engineered to express at least one phosphoglucoisomerase, culturing at least one cell population engineered to express at least one allulose 6-phosphate epimerase, culturing at least one cell population engineered to express at least one allulose 6-phosphate phosphatase, and/or culturing at least one cell population engineered to express at least one debranching enzyme. Following lysis of the cells, the cell lysates are combined such that the enzymes are present in a single cell lysate/reaction mixture.

Cell lysates in some embodiments, may be combined with a nutrient. For example, cell lysates may be combined with Na2HPO4, KH2PO4, NH4Cl, NaCl, MgSO4, CaCl2. Examples of other nutrients include, without limitation, magnesium sulfate, magnesium chloride, magnesium orotate, magnesium citrate, manganese chloride, calcium chloride, cobalt chloride, zinc chloride, zinc sulfate, potassium acetate, potassium phosphate monobasic, potassium phosphate dibasic, potassium phosphate tribasic, sodium phosphate monobasic, sodium acetate, sodium chloride, sodium phosphate dibasic, sodium phosphate tribasic, ammonium phosphate monobasic, ammonium phosphate dibasic, ammonium sulfate, ammonium chloride, and ammonium hydroxide.

Cell lysates, in some embodiments, may be combined with a cofactor. For example, cell lysates may be combined with adenosine diphosphate (ADP), adenosine triphosphate (ATP), nicotinamide adenine dinucleotide (NAD+), or other non-protein chemical compounds required for activity of an enzyme (e.g., inorganic ions and coenzymes)

It should be understood that in any one of the methods described herein, the cells may be lysed by any means, including mechanical, chemical, enzymatic, osmotic, and/or thermal lysis. Thus, in certain embodiments, a lysing step and a heating (heat inactivation) step may be combined as a single step of heating the cells to a temperature that lyses the cells and inactivates undesired native enzymatic activities.

In some embodiments, the methods further include heating the cell lysate(s) (or a cell lysate mixture) to a temperature that inactivates undesired native enzymatic activities but does not inactivate any of the thermostable enzymes of the production pathway, to produce a heat-inactivated lysate. The cell lysate(s), in some embodiments, is heated to a temperature of at least 50° C. For example, the cell lysate(s) may be heated to a temperature of at least 55° C., 60° C., 65° C., 70° C., 75° C., 80° C., 85° C., or 90° C. A native enzyme (or other non-thermostable enzyme) is considered inactive, in some embodiments, when its level of activity is reduced by at least 50%. In some embodiments, a native enzyme (or other non-thermostable enzyme) is considered inactive when its level of activity is reduced by at least 55%, 60%, 65%, 70%, 75%, 80%, 85%, 90%, 95%, 98%, 99%, or 100%.

The cell lysate(s) may be heated for a period of time sufficient to inactivate native enzymes (or other non-thermostable enzymes) of the cell. For example, the cell lysate(s) may be heated for at least 2, 3, 4, or 5 minutes. In some embodiments, the cell lysate(s) are heated for longer than 5 minutes. In some embodiments, the cell lysate(s) are heated for a period of time sufficient to reduce the activity of at least some of the native enzymes (or other non-thermostable enzymes) by at least 50% (e.g., at least 60%, 70%, 80%, or 90%).

Following heat inactivation, in some embodiments, at least one (e.g., at least two or at least three) purified enzyme (or partially purified enzyme) is added to the cell lysate/reaction mixture. Thus, a reaction mixture, in some embodiments, includes a combination of enzymes present in the cell lysate (expressed by the engineered host cell(s)), at least one cofactor or nutrient, and at least one purified enzyme. At least one purified enzyme may be selected from the group consisting of α-glucan phosphorylases, phosphoglucomutases, phosphoglucoisomerases, allulose 6-phosphate epimerases, allulose 6-phosphate phosphatases, and debranching enzymes. At least one purified enzyme may be selected from the group consisting of cellodextrin phosphorylases, phosphoglucomutases, phosphoglucoisomerases, allulose 6-phosphate epimerases, allulose 6-phosphate phosphatases, and debranching enzymes. Advantageously, this allows for the incorporation of a purified enzyme that is not part of a cell lysate and which may be commercially obtained, thus, alleviating the need to engineer a cell to express the needed enzyme.

In some embodiments, the methods also include incubating the heat-inactivated lysate(s) in the presence of a polysaccharide and inorganic phosphate to produce allulose. In some embodiments, the heat-inactivated lysates are incubated at a temperature of at least 50° C. In some embodiments, the heat-inactivated lysates are incubated for at least 2 minutes (e.g., at least 3, 4, or 5 minutes). For example, the heat-inactivated lysates may be incubated for 30-60 minutes, with optimized time reaching below 30 minutes, such as 25-30 minutes, 20-25 minutes, 15-20 minutes, 10-15 minutes, 5-10 minutes, 2-5 minutes, or 2-10 minutes. The polysaccharide may be, for example, a starch, cellulose, maltodextrin, and cellodextrin. In some embodiments, biomass is used instead of a polysaccharide. In some embodiments, the polysaccharide is maltodextrin and is present as a component of a compound (e.g., part of the biomass). For example, in some embodiments, the heat-inactivated lysate(s) (e.g., microbial cell lysates) are incubated in the presence of corn pulp and inorganic phosphate to produce allulose (or any other sugar described herein).

Also provided herein are cells and cell lysates used for the production of allulose. Thus, an engineered cell (e.g., bacterial cell, yeast cell, and/or plant cell) or cell lysate(s) of the present disclosure may include at least one (e.g., at least two, at least three, or at least four) enzyme selected from the group consisting of α-glucan phosphorylases, phosphoglucomutases, phosphoglucoisomerases, allulose 6-phosphate epimerases, allulose 6-phosphate phosphatases, and debranching enzymes. In some embodiments, an engineered cell (e.g., bacterial cell, yeast cell, and/or plant cell) or cell lysate(s) of the present disclosure includes at least one (e.g., at least two, at least three, or at least four) enzyme selected from the group consisting of thermostable α-glucan phosphorylases, thermostable phosphoglucomutases, thermostable phosphoglucoisomerases, thermostable allulose 6-phosphate epimerases, thermostable allulose 6-phosphate phosphatases, and thermostable debranching enzymes. Thus, an engineered cell (e.g., bacterial cell, yeast cell, and/or plant cell) or cell lysate(s) of the present disclosure may include at least one (e.g., at least two, at least three, or at least four) enzyme selected from the group consisting of cellodextrin phosphorylases, phosphoglucomutases, phosphoglucoisomerases, allulose 6-phosphate epimerases, allulose 6-phosphate phosphatases, and debranching enzymes. In some embodiments, an engineered cell (e.g., bacterial cell, yeast cell, and/or plant cell) or cell lysate(s) of the present disclosure includes at least one (e.g., at least two, at least three, or at least four) enzyme selected from the group consisting of thermostable cellodextrin phosphorylases, thermostable phosphoglucomutases, thermostable phosphoglucoisomerases, thermostable allulose 6-phosphate epimerases, thermostable allulose 6-phosphate phosphatases, and thermostable debranching enzymes.

Non-limiting examples of enzymes for use in allulose production pathways (described in Table 1) are provided in Table 2 below.

TABLE 2
Exemplary Allulose Pathway Enzymes

| | Enzyme | Enzyme | Gene Bank | |
|---|---|---|---|---|
| Enzyme Used | Code | Name | or Uniprot | Native Organism |
| α-glucan | A1 | TzAgp | WP_010477083 | Thermococcus zilligii |
| phosphorylase | A2 | PfAgp | WP_011012682 | Pyrococcus furiosus |
| | A3 | TcGlgP | AAV68178 | Thermus caldophilus |
| | A4 | PtAgp | WP_013931760 | Pseudothermotoga thermarum |
| | A5 | Tm08495 | AHD18925 | Thermotoga maritima |
| | A6 | AaGlgP | O66932 | Aquifex aeolicus |
| Cellodextrin | C1 | | BAB71818 | Clostridium thermocellum |
| phosphorylase | C2 | | GAE90338 | Clostridium straminisolvens |
| (also referred to as | C3 | | WP_011943512 | Thermotoga RQ2 |
| cellulose phosphorylase) | C4 | | ADM28607 | Ignisphaera aggregans |
| | C5 | | WP_004082399 | Thermotoga maritima |
| | C6 | | WP_013314871 | Spirochaeta thermophila |
| | C7 | | WP_015907054 | Caldicellulosiruptor bescii |
| | C8 | | WP_012548338 | Dictyoglomus thermophilum |
| | C9 | | WP_013297089 | Thermoanaerobacterium |
| | | | | thermosaccharolyticum |
| | C10 | | ACJ76363 | Thermosipho africanus |
| | C11 | | WP_006313788 | Caldisalinibacter kiritimatiensis |
| | C12 | | WP_058486419 | Defluviitalea phaphyphila |
| | C13 | | WP_013429146 | Caldicellulosiruptor kronotskyensis |
| | C14 | | WP_015848606 | Thermococcus sibiricus |
| | C15 | | WP_013129904 | Thermosphaera aggregans |
| Phosphoglucomutase | M1 | Tk1621 | Q68BJ6 | Thermococcus kodakaraensis |
| | M2 | Pk02350 | A0A127B864 | Pyrococcus kukulkanii |
| | M3 | Af0458 | WP_010877965 | Archaeoglobus fulgidus |
| | M4 | CtPgm2 | WP_003517493 | Clostridium thermocellum |
| | M5 | TtPgm2 | WP_011172742 | Thermus thermophilus |
| | M6 | TiManB | WP_022798653 | Thermus islandicus |
| Phosphoglucoisomerase | I1 | PfPgi | P83194 | Pyrococcus furiosus |
| | I2 | Ap0768 | BAA79746 | Aeropyrum pernix |
| | I3 | Cl1150 | WP_015232769 | Caldisphaera lagunensis |
| | I4 | CtPgi | A3DBX9 | Clostridium thermocellum |
| | I5 | TtPgi | WP_041443619 | Thermus thermophilus |
| | I6 | MjPgi | WP_010871130 | Methanococcus jannaschii |
| Allulose 6-phosphate | E1 | BtAlsE | WP_029098887 | Brevibacillus thermoruber |
| epimerase | E2 | Thebr1340 | WP_003868217 | Thermoanaerobacter brockii |
| | E3 | CasuRpe2 | WP_011025758 | Caldanaerobacter subterraneus |
| | E4 | Defds2125 | WP_013008817 | Deferribacter desulfuricans |
| | E5 | Hg1285 | WP_008287078 | Hydrogenivirga sp. 128-5-R1-1 |
| | E6 | Tthe1731 | WP_013298194 | Thermoanerobacterium |
| | | | | thermosaccharolyticum |
| | E7 | Theru00510 | WP_025305325 | Thermocrinis ruber |
| | E8 | That0313 | WP_073071389 | Thermosipho atlanticus |
| | E9 | ThtaRpe | WP_068550718 | Thermosulfidibacter takaii |
| Allulose 6-phosphate | P1 | AaGph | WP_010880861 | Aquifex aeolicus |
| phosphatase | P2 | Ace10099 | WP_011718939 | Acidothermus cellulolyticus |
| | P3 | CsPpaX | WP_009610632 | Caldanaerobacter subterraneus |
| | P4 | Cthe0261 | WP_003512401 | Clostridium thermocellum |
| | P5 | Desku1269 | AEG14852 | Desulfotomaculum kuznetsovii |
| | P6 | Dgeo0096 | ABF44399 | Deinococcus geothermalis DSM 11300 |
| | P7 | Mth1760 | WP_010877362 | Methanothermobacter |
| | | | | thermautotrophicus |
| | P8 | Pho459 | WP_010884566 | Pyrococcus horikoshii Ot3 |
| | P9 | Pmob0141 | WP_012207996 | Petrotoga mobilis |
| | P10 | Tagg0346 | WP_013129214 | Thermosphaera aggregans |
| | P11 | Tfu0224 | AAZ54262 | Thermobifida fusca |
| | P12 | Theet2005 | WP_003870772 | Thermoanaerobacter ethanolicus |
| | P13 | Thein1775 | WP_013908370 | Thermodesulfatator indicus |
| | P14 | TheisHAD | WP_022799086 | Thermus islandicus |
| | P15 | Thewi1735 | WP_014063120 | Thermoanaerobacter wiegelii |
| | P16 | TtC1471 | AAS81813 | Thermus thermophilus |
| | P17 | Vdis0326 | WP_013335457 | Crenarchaeota archaeon |
| | | | | 13_1_40CM_3_53_5 |
| | P18 | Ctn1320 | ACM23496 | Thermotoga neapolitana |
| | P19 | Bf9343 | ABR41712 | Bacteroides fragilis |
| | P20 | Bvu4110 | CAH06673 | Bacteroides vulgatus |

It should be understood that any of the pathways in Table 1 may be used and may include any combination of enzymes selected from Table 2. For example, a α-glucan phosphorylase may be selected from any one of TzAgp, PfAgp, TcGlgP, PtAgp, Tm08495, and AaGlgP and combined with a phosphoglucomutase selected from any one of Tk1621, Pk02350, Af0458, CtPgm2, TtPgm2, and TiManB and combined with any phosphoglucoisomerase in Table 2, any allulose 6-phosphate epimerase in Table 2, and any allulose 6-phosphate phosphatase in Table 2. In other embodiments, at least one α-glucan phosphorylase, at least one phosphoglucomutase, at least one phosphoglucoisomerase, at least one allulose 6-phosphate epimerase, and at least one allulose 6-phosphate phosphatase are used and selected from the enzymes appearing in Table 2. In other embodiments, enzymes from Table 2 are used in a combination of steps from Table 1 (e.g., steps 1 and 2 are carried out where a α-glucan phosphorylase is used in combination with a phosphoglucomutase to convert a polysaccharide to glucose 6-phosphate). In some embodiments, 2 steps are employed to perform a transformation. In other embodiments, only 3, 4, or 5 steps selected from the group containing Step 1, Step 2, Step 3, Step 4, and Step 5 are used to perform the corresponding transformations. In some embodiments, enzymes from Table 2 are used only to carry out a single step from Table 1 (e.g., only step 2 is carried out were a phosphoglucomutase such as Tk1621 is used to convert glucose 1-phosphate to glucose 6-phosphate). In other embodiment, only a single step is carried out selected from the group consisting of Step 1, Step 2, Step 3, Step 4, and Step 5.

### Substrate Flexibility and Debranching Enzymes

For all pathways described herein, a multitude of polysaccharide substrates can be used. Non-limiting examples of polymeric glucose substrates include starch, glycogen, and maltodextrin. In some embodiments, the substrate is starch. In other embodiments, the substrate is glycogen. In still other embodiments, the substrate is maltodextrin. In some embodiments, a partially hydrolyzed version of a polymeric glucose substrate (e.g., starch, glycogen, or maltodextrin) is used. Starch, glycogen, and maltodextrin include a plurality of glucose monomers linked primarily by α(1-4) bonds and some α(1-6) bonds. Both starch and glycogen contain these α(1-6) branch points, although glycogen is substantially more branched than starch. For the α(1-4) polymers, α-glucan phosphorylases consume the polymers one glucose at a time releasing glucose 1-phosphate.

Long polymers of starch are often insoluble in aqueous solutions and in addition to precipitating out, can cause gelling and retrogradation of the solution. When starch is partially hydrolyzed to smaller chain length polymers, either through chemical (e.g., acid hydrolysis) or enzymatic (e.g., α-amylase) methods, the resulting products are maltodextrins. These hydrolyzed derivatives often solubilize and mix better than their parent molecules, and thus, in some embodiments, are used in the pathways provided herein.

For glycogen, starch, or hydrolyzed maltodextrins, α(1-6) branches will substantially reduce yields of any allulose production pathway, as the glucan phosphorylase chew the polymers down to the end of their branches, leaving a large central core of available glucose unconverted. For these substrates/pathways, debranching enzymes may be used to increase substrate availability to the α-glucan phosphorylase. There are two exemplary classes of debranching enzymes that can be used: isoamylases and pullulanases (Table 3). Enzymatically, both classes perform the same function but differ in substrate specificity. While using the debranching enzyme increases yields, the timing of the use will depend on the process and substrates being used. In some embodiments, an α-glucan is pretreated with α-amylase and a debranching enzyme, and then the resulting debranched maltodextrin(s) is fed into a reactor with the other pathway enzymes. In other embodiments, the debranching occurs concurrent with the pathway and branched α-glucans are fed into the reaction containing all pathway enzymes as well as the debranching enzyme.

Some aspects of the present disclosure provide methods, compositions, and systems for producing allulose. These methods, in some embodiments, include culturing cells engineered to express at least one debranching enzyme, at least one α-glucan phosphorylase, at least one phosphoglucomutase, at least one phosphoglucoisomerase, at least one allulose 6-phosphate epimerase, at least one allulose 6-phosphate phosphatase, or a combination of at least two (e.g., at least three, or at least four) of the foregoing enzymes. These methods, in some embodiments, include culturing cells engineered to express at least one debranching enzyme, at least one cellodextrin phosphorylase, at least one phosphoglucomutase, at least one phosphoglucoisomerase, at least one allulose 6-phosphate epimerase, at least one allulose 6-phosphate phosphatase, or a combination of at least two (e.g., at least three, or at least four) of the foregoing enzymes.

In some embodiments, at least one of the enzymes used to convert a polysaccharide to allulose is a thermostable enzyme. In some embodiments, at least two (e.g., at least three or at least four) of the enzymes are thermostable enzymes. In some embodiments, all of the enzymes are thermostable enzymes. Thus, in some embodiments, the methods include culturing cells engineered to express at least one thermostable debranching enzyme, at least one thermostable α-glucan phosphorylase, at least one thermostable phosphoglucomutase, at least one thermostable phosphoglucoisomerase, at least one thermostable allulose 6-phosphate epimerase, at least one thermostable allulose 6-phosphate phosphatase, or a combination of at least two or more of the foregoing thermostable enzymes.

In some embodiments, at least one of the enzymes used to convert cellulose/cellodextrin to allulose is a thermostable enzyme. In some embodiments, at least two (e.g., at least three or at least four) of the enzymes are thermostable enzymes. In some embodiments, all of the enzymes are thermostable enzymes. Thus, in some embodiments, the methods include culturing cells engineered to express at least one thermostable debranching enzyme, at least one thermostable cellodextrin phosphorylase, at least one thermostable phosphoglucomutase, at least one thermostable phosphoglucoisomerase, at least one thermostable allulose 6-phosphate epimerase, at least one thermostable allulose 6-phosphate phosphatase, or a combination of at least two or more of the foregoing thermostable enzymes.

In some embodiments, the methods of producing allulose include lysing (e.g., thermal, osmotic, mechanical, chemical, or enzymatic lysis) the cultured cells to produce at least one (e.g., at least two, at least three, or at least four) cell lysate. It should be understood that multiple cell lysates (and thus multiple cell populations, e.g., from the same organism (e.g., bacteria) or from different organisms (e.g., bacteria, yeast, and/or plant cells)) may be used in an enzymatic reaction as provided herein. For example, one cell population may be engineered to express one or more enzymes(s) of the allulose production pathway, while another cell population (or several other cell populations) may be engineered to express another (at least one other) enzyme of the allulose production pathway. Thus, in some embodiments, the methods comprise culturing at least one population of cells engineered to express at least one debranching enzyme, culturing at least one population of cells engineered to express at least one α-glucan phosphorylase, culturing at least one cell population engineered to express at least one phosphoglucomutase, culturing at least one cell population engineered to express at least one phosphoglucoisomerase, culturing at least one cell population engineered to express at least one allulose 6-phosphate epimerase, and/or culturing at least one cell population engineered to express at least one allulose 6-phosphate phosphatase. Thus, in some embodiments, the methods comprise culturing at least one population of cells engineered to express at least one debranching enzyme, culturing at least one population of cells engineered to express at least one cellodextrin phosphorylase, culturing at least one cell population engineered to express at least one phosphoglucomutase, culturing at least one cell population engineered to express at least one phosphoglucoisomerase, culturing at least one cell population engineered to express at least one allulose 6-phosphate epimerase, and/or culturing at least one cell population engineered to express at least one allulose 6-phosphate phosphatase. Following lysis of the cells, the cell lysates are combined such that the enzymes are present in a single cell lysate/reaction mixture.

Also provided herein are cells and cell lysates used for the production of allulose. Thus, an engineered cell (e.g., bacterial cell, yeast cell, and/or plant cell) or cell lysate(s) of the present disclosure may include at least one (e.g., at least two, at least three, or at least four) enzyme selected from the group consisting of debranching enzymes, α-glucan phosphorylases, phosphoglucomutases, phosphoglucoisomerases, allulose 6-phosphate epimerases, and allulose 6-phosphate phosphatases. In some embodiments, an engineered cell (e.g., bacterial cell, yeast cell, and/or plant cell) or cell lysate(s) of the present disclosure includes at least one (e.g., at least two, at least three, or at least four) enzyme selected from the group consisting of thermostable debranching enzymes, thermostable α-glucan phosphorylases, thermostable phosphoglucomutases, thermostable phosphoglucoisomerases, thermostable allulose 6-phosphate epimerases, and thermostable allulose 6-phosphate phosphatases.

Also provided herein are cells and cell lysates used for the production of allulose. Thus, an engineered cell (e.g., bacterial cell, yeast cell, and/or plant cell) or cell lysate(s) of the present disclosure may include at least one (e.g., at least two, at least three, or at least four) enzyme selected from the group consisting of debranching enzymes, cellodextrin phosphorylases, phosphoglucomutases, phosphoglucoisomerases, allulose 6-phosphate epimerases, and allulose 6-phosphate phosphatases. In some embodiments, an engineered cell (e.g., bacterial cell, yeast cell, and/or plant cell) or cell lysate(s) of the present disclosure includes at least one (e.g., at least two, at least three, or at least four) enzyme selected from the group consisting of thermostable debranching enzymes, thermostable cellodextrin phosphorylases, thermostable phosphoglucomutases, thermostable phosphoglucoisomerases, thermostable allulose 6-phosphate epimerases, and thermostable allulose 6-phosphate phosphatases.

Non-limiting examples of debranching enzymes for use in allulose production pathways (described in Table 1) are provided in Table 3 below.

TABLE 3
Exemplary Debranching Enzymes

| Debranching | Enzyme | GeneBank or | |
|---|---|---|---|
| Enzyme | Name | Uniprot | Native Organism |
| Pullulanase | Fp1793 | AAD30387 | Fervidobacterium |
| | | | pennavorans |
| | TRQ5Pul | WP_012310857 | Thermotoga sp. RQ5 |
| | BfPull | BAB18516 | Bacillus |
| | | | flavocaldarius |
| Isoamylase | StTreX | BAB65940 | Sulfolobus tokodaii |
| | MhTreX | AAS00512 | Metallosphaera |
| | | | hakonensis |
| | StGlgX | WP_012873143 | Sphaerobacter |
| | | | thermophilus |

It should be understood that any of the pathways in Table 1 may be used for producing allulose and may include any combination of enzymes selected from Table 2. For example, a α-glucan phosphorylase may be selected from any one of TzAgp, PfAgp, TcGlgP, PtAgp, Tm08495, and AaGlgP and combined with a phosphoglucomutase selected from any one of Tk1621, Pk02350, Af0458, CtPgm2, TtPgm2, and TiManB and combined with any phosphoglucoisomerase in Table 2, any allulose 6-phosphate epimerase in Table 2, any allulose 6-phosphate phosphatase in Table 2, and further comprise any enzymes selected from Table 3, such as a pullulanase or isoamylase. In other embodiments, at least one debranching enzyme, at least one α-glucan phosphorylase, at least one phosphoglucomutase, at least one phosphoglucoisomerase, at least one allulose 6-phosphate epimerase, and at least one allulose 6-phosphate phosphatase are used and selected from the enzymes appearing in Table 2 and Table 3. In other embodiments, enzymes from Table 2 and 3 are used in a combination of steps from Table 1 (e.g., steps D1, 1, and 2 are used in combination where a α-glucan phosphorylase (e.g., TzAgp) and a phosphoglucomutase (e.g., Tk1621) are used in combination with a debranching enzyme such as StGlgX to convert a polysaccharide to glucose 6-phosphate). In some embodiments, 2 steps are employed to perform a set of transformations. In other embodiments, only 3, 4, 5, 6, or 7 steps selected from the group containing Step D1, Step D2, Step 1, Step 2, Step 3, Step 4, and Step 5 are used to perform the corresponding transformations. In some embodiments, enzymes from Table 3 are used only to carry out a single step from Table 1 (e.g., only step D2 is performed where StTreX is used for debranching). In other embodiment, only a single step is carried out selected from the group consisting of Step D1, Step D2, Step 1, Step 2, Step 3, Step 4, and Step 5.

### Cell-Free Production

“Cell-free production” is the use of biological processes for the synthesis of a biomolecule or chemical compound without using living cells. Rather, the cells are lysed and unpurified (crude) portions, containing enzymes, are used for the production of a desired product. As a non-limiting example, cells are cultured, harvested, and lysed by high-pressure homogenization. The cell-free reaction may be conducted in a batch or fed-batch mode. In some instances, the biological reaction networks fill the working volume of the reactor and may be more dilute than the intracellular environment. Yet substantially all of the cellular catalysts are provided, including catalysts that are membrane associated. The inner membrane is fragmented during cell lysis, and the fragments of these membranes form functional membrane vesicles. Thus, complex biotransformations are effected by catalysis. See, e.g., Swartz, AIChE Journal, 2012, 58(1), 5-13, incorporated herein by reference.

Cell-free methods and systems of the present disclosure, in some embodiments, utilize cell lysates (e.g., crude or partially purified cell lysates), discussed in greater detail herein. Cell lysates may be prepared, for example, by mechanical means (e.g., shearing or crushing). In some embodiments, cell lysates are distinct from chemically-permeabilized cells. As discussed here, in some embodiments, during cell lysis (e.g., mechanical cell lysis), the inner cell membrane is fragmented such that inverted membrane vesicles are formed in the cells lysates. Cells that are lysed (e.g., at least 75%, 80%, 85%, 90%, or 95%) are no longer intact.

In some embodiments, permeabilized cells are used. Permeabilized cells are intact cells containing perforations (small holes). In some embodiments, cells may be permeabilized to release the cell content for use in a reaction as provided herein.

In some embodiments, partially purified cell fractions are used. A partially purified cell fraction is a cell lysate from which one or more cellular components (e.g., cell membranes) have been partially or completely removed.

### Thermostable Enzymes

An enzyme is considered thermostable if the enzyme (a) retains a substantial portion of its activity after exposure to high temperatures that denature other native enzymes or (b) functions at a relatively high rate after exposure to a medium to high temperature where native enzymes function at low rates.

In some embodiments, a thermostable enzyme retains greater than 50% activity following exposure to relatively high temperature that would otherwise denature a similar (non-thermostable) native enzyme. In some embodiments, a thermostable enzyme retains 50-100% activity following exposure to relatively high temperature that would otherwise denature a similar (non-thermostable) native enzyme. For example, a thermostable enzyme may retain 50-90%, 50-85%, 50-80%, 50-75%, 50-70%, 50-65%, 50-60%, or 50-55% of its activity following exposure to relatively high temperature that would otherwise denature a similar (non-thermostable) native enzyme. In some embodiments, a thermostable enzyme retains 25%, 30%, 35%, 40%, 45%, 50%, 55%, 60%, 65%, 70%, 75%, 80%, 85%, 90%, 95%, 98%, 99%, or 100% of its activity following exposure to relatively high temperature that would otherwise denature a similar (non-thermostable) native enzyme.

In some embodiments, the activity of a thermostable enzyme after exposure medium to high temperature is greater than (e.g., 25%, 30%, 35%, 40%, 45%, 50%, 55%, 60%, 65%, 70%, 75%, 80%, 85%, 90%, 95%, 98%, 99%, or 100% greater than) the activity of a similar (non-thermostable) native enzyme.

Thermostable enzymes (e.g., phosphatases or phosphorylases) may remain active (able to catalyze a reaction), for example, at temperatures of 45° C. to 80° C., or higher. In some embodiments, thermostable enzymes remain active at a temperature of 45-80° C., 45-70° C., 45-60° C., 45-50° C., 50-80° C., 50-70° C., 50-60° C., 60-80° C., 60-70° C., or 70-80° C. For example, thermostable enzymes may remain active at a temperature of 45° C., 46° C., 47° C., 48° C., 49° C., 50° C., 51° C., 52° C., 53° C., 54° C., 55° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., or 80° C. Thermostable enzymes may remain active at relatively high temperatures for 15 minutes to 48 hours, or longer, after exposure to relatively high temperatures. For example, thermostable enzymes may remain active at relatively high temperatures for 1, 2, 3, 4, 5, 6, 7, 8, 9, 10, 11, 12, 13, 14, 15, 16, 17, 18, 19, 20, 24, 36, 42, or 48 hours.

### Engineered Cells

Engineered cells of the present disclosure, in some embodiments, comprise at least one, or all, of the enzymatic activities required to convert a polysaccharide and/or starch and/or maltodextrin to allulose. “Engineered cells” are cells that comprise at least one engineered (e.g., recombinant or synthetic) nucleic acid, or are otherwise modified such that they are structurally and/or functionally distinct from their naturally-occurring counterparts. Thus, a cell that contains an engineered nucleic acid is considered an “engineered cell.”

Engineered cells of the present disclosure, in some embodiments, comprise an α-glucan phosphorylase (e.g., a thermostable α-glucan phosphorylase), a phosphoglucomutase (e.g., a thermostable phosphoglucomutase), and at least one enzyme (e.g., thermostable enzyme) selected from the group consisting of phosphoglucoisomerases, allulose 6-phosphate 3-epimerases, allulose 6-phosphate phosphatases, and optionally a debranching enzyme.

Engineered cells of the present disclosure, in some embodiments, comprise a cellodextrin phosphorylase (e.g., a thermostable cellodextrin phosphorylase), a phosphoglucomutase (e.g., a thermostable phosphoglucomutase), and at least one enzyme (e.g., thermostable enzyme) selected from the group consisting of phosphoglucoisomerases, allulose 6-phosphate 3-epimerases, allulose 6-phosphate phosphatases, and optionally a debranching enzyme.

Engineered cells, in some embodiments, express selectable markers. Selectable markers are typically used to select engineered cells that have taken up and express an engineered nucleic acid following transfection of the cell (or following other procedures used to introduce foreign nucleic acid into the cell). Thus, a nucleic acid encoding product may also encode a selectable marker. Examples of selectable markers include, without limitation, antibiotic resistance free markers, genes encoding proteins that increase or decrease either resistance or sensitivity to antibiotics (e.g., ampicillin resistance genes, kanamycin resistance genes, neomycin resistance genes, tetracycline resistance genes and chloramphenicol resistance genes) or other compounds. Other selectable markers may be used in accordance with the present disclosure.

An engineered cell “expresses” a product if the product, encoded by a nucleic acid (e.g., an engineered nucleic acid), is produced in the cell. It is known in the art that gene expression refers to the process by which genetic instructions in the form of a nucleic acid are used to synthesize a product, such as a protein (e.g., an enzyme).

Engineered cells may be prokaryotic cells or eukaryotic cells. In some embodiments, engineered cells are bacterial cells, yeast cells, insect cells, mammalian cells, or other types of cells.

Engineered bacterial cells useful in the present disclosure include, without limitation, engineered Escherichia spp., Streptomyces spp., Zymonas spp., Acetobacter spp., Citrobacter spp., Synechocystis spp., Rhizobium spp., Clostridium spp., Corynebacterium spp., Streptococcus spp., Xanthomonas spp., Lactobacillus spp., Lactococcus spp., Bacillus spp., Alcaligenes spp., Pseudomonas spp., Aeromonas spp., Azotobacter spp., Comamonas spp., Mycobacterium spp., Rhodococcus spp., Gluconobacter spp., Ralstonia spp., Acidithiobacillus spp., Microlunatus spp., Geobacter spp., Geobacillus spp., Arthrobacter spp., Flavobacterium spp., Serratia spp., Saccharopolyspora spp., Thermus spp., Stenotrophomonas spp., Chromobacterium spp., Sinorhizobium spp., Saccharopolyspora spp., Agrobacterium spp., Vibrio spp., and Pantoea spp.

Engineered yeast cells useful in the present disclosure include, without limitation, engineered Saccharomyces spp., Schizosaccharomyces, Hansenula, Candida, Kluyveromyces, Yarrowia and Pichia.

In some embodiments, engineered cells useful in the present disclosure are engineered Escherichia coli cells, Bacillus subtilis cells, Pseudomonas putida cells, Saccharomyces cerevisiae cells, and/or Lactobacillus brevis cells. In some embodiments, engineered cells useful in the present disclosure are engineered Escherichia coli cells.

### Engineered Nucleic Acids

A “nucleic acid” is at least two nucleotides covalently linked together, and in some instances, may contain phosphodiester bonds (e.g., a phosphodiester “backbone”). Nucleic acids (e.g., components, or portions, of nucleic acids) may be naturally occurring or engineered. “Naturally occurring” nucleic acids are present in a cell that exists in nature in the absence of human intervention. “Engineered nucleic acids” include recombinant nucleic acids and synthetic nucleic acids. A “recombinant nucleic acid” refers to a molecule that is constructed by joining nucleic acid molecules (e.g., from the same species or from different species) and, typically, can be replicated in a living cell. A “synthetic nucleic acid” refers to a molecule that is biologically synthesized, chemically synthesized, or by other means synthesized or amplified. A synthetic nucleic acid includes nucleic acids that are chemically modified or otherwise modified but can base pair with naturally-occurring nucleic acid molecules. Recombinant and synthetic nucleic acids also include those molecules that result from the replication of either of the foregoing. Engineered nucleic acids may contain portions of nucleic acids that are naturally occurring, but as a whole, engineered nucleic acids do not occur naturally and require human intervention. In some embodiments, a nucleic acid encoding a product of the present disclosure is a recombinant nucleic acid or a synthetic nucleic acid. In other embodiments, a nucleic acid encoding a product is naturally occurring.

An engineered nucleic acid encoding enzymes, as provided herein, may be operably linked to a “promoter,” which is a control region of a nucleic acid at which initiation and rate of transcription of the remainder of a nucleic acid are controlled. A promoter drives expression or drives transcription of the nucleic acid that it regulates.

A promoter may be one naturally associated with a gene or sequence, as may be obtained by isolating the 5′ non-coding sequences located upstream of the coding segment of a given gene or sequence. Such a promoter can be referred to as “endogenous.”

In some embodiments, a coding nucleic acid sequence may be positioned under the control of a recombinant or heterologous promoter, which refers to a promoter that is not normally associated with the encoded sequence in its natural environment. Such promoters may include promoters of other genes; promoters isolated from any other cell; and synthetic promoters or enhancers that are not “naturally occurring” such as, for example, those that contain different elements of different transcriptional regulatory regions and/or mutations that alter expression through methods of genetic engineering that are known in the art. In addition to producing nucleic acid sequences of promoters and enhancers synthetically, sequences may be produced using recombinant cloning and/or nucleic acid amplification technology, including polymerase chain reaction (PCR).

A promoter is considered to be “operably linked” when it is in a correct functional location and orientation in relation to the nucleic acid it regulates to control (“drive”) transcriptional initiation and/or expression of that nucleic acid.

Engineered nucleic acids of the present disclosure may contain a constitutive promoter or an inducible promoter. A “constitutive promoter” refers to a promoter that is constantly active in a cell. An “inducible promoter” refers to a promoter that initiates or enhances transcriptional activity when in the presence of, influenced by, or contacted by an inducer or inducing agent, or activated in the absence of a factor that causes repression. Inducible promoters for use in accordance with the present disclosure include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters, steroid-regulated promoters, metal-regulated promoters, pathogenesis-regulated promoters, temperature/heat-inducible, phosphate-regulated (e.g., PhoA), and light-regulated promoters.

An inducer or inducing agent may be endogenous or a normally exogenous condition (e.g., light), compound (e.g., chemical or non-chemical compound) or protein that contacts an inducible promoter in such a way as to be active in regulating transcriptional activity from the inducible promoter. Thus, a “signal that regulates transcription” of a nucleic acid refers to an inducer signal that acts on an inducible promoter. A signal that regulates transcription may activate or inactivate transcription, depending on the regulatory system used. Activation of transcription may involve directly acting on a promoter to drive transcription or indirectly acting on a promoter by inactivation of a repressor that is preventing the promoter from driving transcription. Conversely, deactivation of transcription may involve directly acting on a promoter to prevent transcription or indirectly acting on a promoter by activating a repressor that then acts on the promoter.

Engineered nucleic acids may be introduced into host cells using any means known in the art, including, without limitation, transformation, transfection (e.g., chemical (e.g., calcium phosphate, cationic polymers, or liposomes) or non-chemical (e.g., electroporation, sonoporation, impalefection, optical transfection)), and transduction (e.g., viral transduction).

Enzymes or other proteins encoded by a naturally-occurring, intracellular nucleic acid may be referred to as “endogenous enzymes” or “endogenous proteins.”

### Protease Targeting

Engineered cells of the present disclosure may express (e.g., endogenously express) enzymes necessary for the health of the cells that may have a negative impact on the production of a sugar of interest (e.g., allulose). Such enzymes are referred to herein as “target enzymes.” For example, target enzymes expressed by engineered cells may compete for substrates or cofactors with an enzyme that increases the rate of precursor supplied to a sugar production pathway. As another example, target enzymes expressed by the engineered cells may compete for substrates or cofactors with an enzyme that is a key pathway entry enzyme of a sugar production pathway. As yet another example, target enzymes expressed by the engineered cells may compete for substrates or cofactors with an enzyme that supplies a substrate or cofactor of a sugar production pathway.

To negate, or reduce, this negative impact, target enzymes can be modified to include a site-specific protease-recognition sequence in their protein sequence such that the target enzyme may be “targeted” and cleaved for inactivation during sugar production (see, e.g., U.S. Publication No. 2012/0052547 A1, published on Mar. 1, 2012; and International Publication No. WO 2015/021058 A2, published Feb. 12, 2015, each of which is incorporated by reference herein).

Cleavage of a target enzyme containing a site-specific protease-recognition sequence results from contact with a cognate site-specific protease that is sequestered in the periplasm of cell (separate from the target enzyme) during the cell growth phase (e.g., as engineered cells are cultured) and is brought into contact with the target enzyme during the conversion phase (e.g., following cell lysis to produce a cell lysate). Thus, engineered cells of the present disclosure comprise, in some embodiments, (i) an engineered nucleic acid encoding a target enzyme that negatively impacts the rate of conversion and includes a site-specific protease-recognition sequence in the protein sequence of the target enzyme, and (ii) an engineered nucleic acid encoding a site-specific protease that cleaves the site-specific protease-recognition sequence of the target enzyme and includes a periplasmic-targeting sequence. This periplasmic-targeting sequence is responsible for sequestering the site-specific protease to the periplasmic space of the cell until the cell is lysed. Examples of periplasmic-targeting sequences are provided below.

Examples of proteases that may be used in accordance with the present disclosure include, without limitation, alanine carboxypeptidase, astacin, bacterial leucyl aminopeptidase, cancer procoagulant, cathepsin B, clostripain, cytosol alanyl aminopeptidase, elastase, endoproteinase Brg-C, enterokinase, gastricsin, gelatinase, Gly-X carboxypeptidase, glycyl endopeptidase, human rhinovirus 3C protease, hypodermin C, Iga-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2B, picornain 3C, proendopeptidase, prolyl aminopeptidase, proprotein convertase I, proprotein convertase II, russellysin, saccharopepsin, semenogelase, T-plasminogen activator, thrombin, tissue kallikrein, tobacco etch virus (TEV), togavirin, tryptophanyl aminopeptidase, U-plasminogen activator, V8, venombin B, venombin BB and Xaa-pro aminopeptidase.

### Periplasmic Targeting

Enzymes of an allulose production pathway may include at least one enzyme that has a negative impact on the health (e.g., viability) of a cell. To negate or reduce this negative impact, an enzyme can be modified to include a relocation sequence such that the enzyme is relocated to a cellular or extra-cellular compartment where it is not naturally located and where the enzyme does not negatively impact the health of the cell (see, e.g., Publication No. US-2011-0275116-A1, published on Nov. 10, 2011, incorporated by reference herein). For example, an enzyme of an allulose production pathway may be relocated to the periplasmic space of a cell.

Thus, in some embodiments, engineered cells of the present disclosure comprise at least one enzyme of an allulose production pathway that is linked to a periplasmic-targeting sequence. A “periplasmic-targeting sequence” is an amino acid sequence that targets to the periplasm of a cell the protein to which it is linked. A protein that is linked to a periplasmic-targeting sequence will be sequestered in the periplasm of the cell in which the protein is expressed.

Periplasmic-targeting sequences may be derived from the N-terminus of bacterial secretory protein, for example. The sequences vary in length from about 15 to about 70 amino acids. The primary amino acid sequences of periplasmic-targeting sequences vary, but generally have a common structure, including the following components: (i) the N-terminal part has a variable length and generally carries a net positive charge; (ii) following is a central hydrophobic core of about 6 to about 15 amino acids; and (iii) the final component includes four to six amino acids which define the cleavage site for signal peptidases.

Periplasmic-targeting sequences of the present disclosure, in some embodiments, may be derived from a protein that is secreted in a gram-negative bacterium. The secreted protein may be encoded by the bacterium, or by a bacteriophage that infects the bacterium. Examples of gram-negative bacterial sources of secreted proteins include, without limitation, members of the genera Escherichia, Pseudomonas, Klebsiella, Salmonella, Caulobacter, Methylomonas, Acetobacter, Achromobacter, Acinetobacter, Aeromonas, Agrobacterium, Alcaligenes, Azotobacter, Burkholderia, Citrobacter, Comamonas, Enterobacter, Erwinia, Rhizobium, Vibrio, and Xanthomonas.

Examples of periplasmic-targeting sequences for use in accordance with the present disclosure include, without limitation, sequences selected from the group consisting of:

(SEQ ID NO: 1)
MKIKTGARILALSALTTMMFSASALA;
(SEQ ID NO: 2)
MKQSTIALALLPLLFTPVTKA;
(SEQ ID NO: 3)
MMITLRKLPLAVAVAAGVMSAQAMA;
(SEQ ID NO: 4)
MNKKVLTLSAVMASMLFGAAAHA;
(SEQ ID NO: 5)
MKYLLPTAAAGLLLLAAQPAMA;
(SEQ ID NO: 6)
MKKIWLALAGLVLAFSASA;
(SEQ ID NO: 7)
MMTKIKLLMLIIFYLIISASAHA;
(SEQ ID NO: 8)
MKQALRVAFGFLILWASVLHA;
(SEQ ID NO: 9)
MRVLLFLLLSLFMLPAFS; and
(SEQ ID NO: 10)
MANNDLFQASRRRFLAQLGGLTVAGMLGPSLLTPRRATA.

### Fusion or Bifunctional Protein

In some embodiments, the α-glucan phosphorylase and the phosphoglucomutase are expressed as a single fusion (chimeric) protein or a bifunctional protein. A fusion protein may be created by joining two or more genes or gene segments that code for separate proteins. Translation of this fusion gene results in a single or multiple polypeptides with functional properties derived from each of the original proteins. A polyfunctional protein is a single protein that has at least two different activities, wherein that functionality is a native biological function or the result of an engineered enzyme fusion. Other enzymes may also be expressed as a single fusion protein or a polyfunctional protein. Thus, a fusion protein may contain multiple functionalities of any of the pathway enzymes described herein.

### Cell Cultures and Cell Lysates

Typically, engineered cells are cultured. “Culturing” refers to the process by which cells are grown under controlled conditions, typically outside of their natural environment. For example, engineered cells, such as engineered bacterial cells, may be grown as a cell suspension in liquid nutrient broth, also referred to as liquid “culture medium.” In some embodiments, unconverted starch is used as a substrate feed for growing cells.

Examples of commonly used bacterial Escherichia coli growth media include, without limitation, LB (Luria Bertani) Miller broth (1% NaCl): 1% peptone, 0.5% yeast extract, and 1% NaCl; LB (Luria Bertani) Lennox Broth (0.5% NaCl): 1% peptone, 0.5% yeast extract, and 0.5% NaCl; SOB medium (Super Optimal Broth): 2% peptone, 0.5% Yeast extract, 10 mM NaCl, 2.5 mM KCl, 10 mM MgCl2, 10 mM MgSO4; SOC medium (Super Optimal broth with Catabolic repressor): SOB+20 mM glucose; 2× YT broth (2× Yeast extract and Tryptone): 1.6% peptone, 1% yeast extract, and 0.5% NaCl; TB (Terrific Broth) medium: 1.2% peptone, 2.4% yeast extract, 72 mM K2HPO4, 17 mM KH2PO4 and 0.4% glycerol; and SB (Super Broth) medium: 3.2% peptone, 2% yeast extract, and 0.5% NaCl and or Korz medium (Korz, D J et al. 1995).

Examples of high density bacterial Escherichia coli growth media include, but are not limited to, DNAGro™ medium, ProGro™ medium, AutoX™ medium, DetoX™ medium, InduX™ medium, and SecPro™ medium.

In some embodiments, engineered cells are cultured under conditions that result in expression of enzymes or nucleic acids. Such culture conditions may depend on the particular product being expressed and the desired amount of the product.

In some embodiments, engineered cells are cultured at a temperature of 30° C. to 40° C. For example, engineered cells may be cultured at a temperature of 30° C., 31° C., 32° C., 33° C., 34° C., 35° C., 36° C., 37° C., 38° C., 39° C., or 40° C. Typically, engineered cells, such as engineered bacterial cells, are cultured at a temperature of 37° C.

In some embodiments, engineered cells are cultured for a period of time of 12 hours to 72 hours, or more. For example, engineered cells may be cultured for a period of time of 12, 18, 24, 30, 36, 42, 48, 54, 60, 66, or 72 hours. Typically, engineered cells, such as engineered bacterial cells, are cultured for a period of time of 12 to 24 hours. In some embodiments, engineered cells are cultured for 12 to 24 hours at a temperature of 37° C.

In some embodiments, engineered cells are cultured (e.g., in liquid cell culture medium) to an optical density, measured at a wavelength of 600 nm (0D600), of 5 to 25. In some embodiments, engineered cells are cultured to an OD600 of 5, 10, 15, 20, or 25.

In some embodiments, engineered cells are cultured to a density of 1×10⁴ to 1×10⁸ viable cells/ml cell culture medium. In some embodiments, engineered cells are cultured to a density of 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 1×10⁹, or 1×10¹⁰ viable cells/ml. In some embodiments, engineered cells are cultured to a density of 1×10⁸ to 1×10¹⁰ viable cells/ml. In some embodiments, engineered cells are cultured to a density of 2×10⁵ to 3×10⁷ viable cells/ml.

In some embodiments, engineered cells are cultured in a bioreactor. A bioreactor refers simply to a container in which cells are cultured, such as a culture flask, a dish, or a bag that may be single-use (disposable), autoclavable, or sterilizable. The bioreactor may be made of glass, or it may be polymer-based, or it may be made of other materials.

Examples of bioreactors include, without limitation, stirred tank (e.g., well mixed) bioreactors and tubular (e.g., plug flow) bioreactors, airlift bioreactors, membrane stirred tanks, spin filter stirred tanks, vibromixers, fluidized bed reactors, and membrane bioreactors. The mode of operating the bioreactor may be a batch or continuous processes and will depend on the engineered cells being cultured. A bioreactor is continuous when the feed and product streams are continuously being fed and withdrawn from the system. A batch bioreactor may have a continuous recirculating flow, but no continuous feeding of nutrient or product harvest. For intermittent-harvest and fed-batch (or batch fed) cultures, cells are inoculated at a lower viable cell density in a medium that is similar in composition to a batch medium. Cells are allowed to grow exponentially with essentially no external manipulation until nutrients are somewhat depleted and cells are approaching stationary growth phase. At this point, for an intermittent harvest batch-fed process, a portion of the cells and product may be harvested, and the removed culture medium is replenished with fresh medium. This process may be repeated several times. For production of recombinant proteins, a fed-batch process may be used. While cells are growing exponentially, but nutrients are becoming depleted, concentrated feed medium (e.g., 10-15 times concentrated basal medium) is added either continuously or intermittently to supply additional nutrients, allowing for further increase in cell concentration and the length of the conversion phase. Fresh medium may be added proportionally to cell concentration without removal of culture medium (broth). To accommodate the addition of medium, a fed-batch culture is started in a volume much lower that the full capacity of the bioreactor (e.g., approximately 40% to 50% of the maximum volume).

Some methods of the present disclosure are directed to large-scale production of sugar. For large-scale production methods, engineered cells may be grown in liquid culture medium in a volume of 5 liters (L) to 50 L, or more. In some embodiments, engineered cells may be grown in liquid culture medium in a volume of greater than (or equal to) 10 L. In some embodiments, engineered cells are grown in liquid culture medium in a volume of 5 L, 10 L, 15 L, 20 L, 25 L, 30 L, 35 L, 40 L, 45 L, 50 L, or more. In some embodiments, engineered cells may be grown in liquid culture medium in a volume of 5 L to 10 L, 5 L to 15 L, 5 L to 20 L, 5 L to 25 L, 5 L to 30 L, 5 L to 35 L, 5 L to 40 L, 5 L to 45 L, 10 L to 15 L, 10 L to 20 L, 10 L to 25 L, 20 L to 30 L, 10 L to 35 L, 10 L to 40 L, 10 L to 45 L, 10 L to 50 L, 15 L to 20 L, 15 L to 25 L, 15 L to 30 L, 15 L to 35 L, 15 L to 40 L, 15 L to 45 L, or 15 to 50 L.

Typically, culturing of engineered cells is followed by lysing the cells. “Lysing” refers to the process by which cells are broken down, for example, by viral, enzymatic, mechanical, chemical, heat or osmotic mechanisms. A “cell lysate” refers to a fluid containing the contents of lysed cells (e.g., lysed engineered cells), including, for example, organelles, membrane lipids, proteins, nucleic acids and inverted membrane vesicles. Cell lysates of the present disclosure may be produced by lysing any population of engineered cells, as provided herein. A “cell lysate” may exclude permeabilized/perforated cells.

Methods of cell lysis, referred to as “lysing,” are known in the art, any of which may be used in accordance with the present disclosure. Such cell lysis methods include, without limitation, physical/mechanical lysis, such as homogenization, as well as chemical, thermal, and/or enzymatic lysis.

Cell lysis can disturb carefully controlled cellular environments, resulting in protein degradation and modification by unregulated endogenous proteases and phosphatases. Thus, in some embodiments, protease inhibitors and/or phosphatase inhibitors may be added to the cell lysate or cells before lysis, or these activities may be removed by gene inactivation or protease targeting.

Cell lysates, in some embodiments, may be combined with at least one nutrient. For example, cell lysates may be combined with Na₂HPO₄, KH₂PO₄, NH₄Cl, NaCl, MgSO₄, CaCl₂. Examples of other nutrients include, without limitation, magnesium sulfate, magnesium chloride, magnesium orotate, magnesium citrate, manganese chloride, calcium chloride, cobalt chloride, zinc chloride, zinc sulfate, potassium acetate, potassium phosphate monobasic, potassium phosphate dibasic, potassium phosphate tribasic, sodium acetate, sodium chloride, sodium phosphate monobasic, sodium phosphate dibasic, sodium phosphate tribasic, ammonium phosphate monobasic, ammonium phosphate dibasic, ammonium sulfate, ammonium chloride, ammonium hydroxide.

In some embodiments, cell lysates may consist of disrupted cell suspensions that are further modified by chemical, thermal, enzymatic or mechanical means to enrich or purify or reduce or eliminate specific components. For example, following disruption via mechanical, thermal, chemical or enzymatic means, as described above, the resulting material may be subjected to mechanical separation, e.g. membrane filtration, centrifugation or others, to partially enrich for a select enzymatic activity or to eliminate an undesired enzymatic activity or lysate component. Further examples may include the addition of salts or solvents to a disrupted cell suspension or alteration of the pH or temperature of the disrupted cell suspension resulting in the precipitation of desired activities followed by mechanical separation of these precipitated components as described above. Conversely, the addition of salts or solvents or the alteration of pH or temperature can be leveraged to eliminate undesired activities through either inactivation of those enzymes or precipitation and subsequent mechanical separation of the undesired enzymatic activity or activities.

Cell lysates, in some embodiments, may be combined with at least one cofactor. For example, cell lysates may be combined with adenosine diphosphate (ADP), adenosine triphosphate (ATP), nicotinamide adenine dinucleotide (NAD+), or other non-protein chemical compounds required for activity of an enzyme (e.g., inorganic ions and coenzymes).

In some embodiments, cell lysates are incubated under conditions that result in conversion of a polysaccharide or starch to sugar.

The volume of cell lysate used for a single reaction may vary. In some embodiments, the volume of a cell lysate is 1 to 150 m³. For example, the volume of a cell lysate may be 1 m³, 5 m³, 10 m³, 15 m³, 20 m³, 25 m³, 30 m³, 35 m³, 40 m³, 45 m³, 50 m³, 55 m³, 60 m³, 65 m³, 70 m³, 75 m³, 80 m³, 85 m³, 90 m³, 95 m³, 100 m³, 105 m³, 110 m³, 115 m³, 120 m³, 125 m³, 130 m³, 135 m³, 140 m³, 145 m³, or 150 m³. In some embodiments, the volume of a cell lysate is 25 m³ to 150 m³, 50 m³ to 150 m³, or 100 m³ to 150 m³.

### Purified Enzymes

In some embodiments of the present invention enzymes may be purified prior to addition to the production reaction. Enzyme purification should be understood to mean any enrichment or extraction of a specific enzyme or enzymatic activity or groups of enzymes or enzymatic activities from a complex mixture of materials, examples including, but not limited to, disrupted cell suspensions or cultured growth media. Thus a purified enzyme or protein should be understood to be an enzyme or protein that has been separated or enriched from a complex matrix, whereby its relative concentration, as compared to other matrix components, is increased. Methods for purifying an enzyme include, but are not limited to, mechanical, chromatographic, chemical, pH or temperature means. For example, the addition of a salt to a disrupted cell suspension resulting in the precipitation of the target enzyme or protein followed by mechanical separation of the precipitated enzyme or protein, e.g., membrane filtration or centrifugation. Further examples may include the separation of an enzyme from a complex matrix through affinity based chromatographic methods (e.g. hexa-histidine-tag or streptavidin based purification).

### Enzymatic Specificity

Enzymatic specificity should be understood to be a trait inherent to an enzyme wherein it demonstrates improved reaction kinetics, thermodynamics or rates for one substrate as compared to another substrate. Enzymes with high specificity for a particular substrate are best exemplified by having a high ratio of catalytic rate (defined as turnover number or k_{cat}) to the Michaelis constant (Kₘ) or k_{cat}/Kₘ as compared to other substrates. It is advantageous to have an enzyme with high substrate specificity as this improves the rate of a reaction and improves yield by decreasing the production of non-target products. For example, the pathway described herein for the production of allulose has several intermediates that are similar in chemical structure, namely glucose 1-phosphate, glucose 6-phosphate, fructose 6-phosphate and allulose 6-phosphate. The ultimate enzymatic step in this process is the dephosphorylation of allulose 6-phosphate to the product allulose via an allulose 6-phosphate phosphatase. It is advantageous to utilize an enzyme with a very high-specificity for allulose 6-phosphate and a relatively low specificity for the other pathway intermediates, namely glucose 1-phosphate, glucose 6-phosphate and fructose 6-phosphate. Catalytic dephosphorylation of these intermediates would result in the production of either glucose or fructose thus decreasing yield and increasing product complexity.

### Kits

The kits described herein may include one or more containers housing components for performing the methods described herein and optionally instructions of uses. Any of the kit described herein may further comprise components needed for performing the methods. Each component of the kits, where applicable, may be provided in liquid form (e.g., in solution), or in solid form, (e.g., a dry powder). In certain cases, some of the components may be reconstitutable or otherwise processible (e.g., to an active form), for example, by the addition of a suitable solvent or other species (e.g., water or buffer), which may or may not be provided with the kit.

In some embodiments, the kits may optionally include instructions and/or promotion for use of the components provided. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. As used herein, “promoted” includes all methods of doing business including methods of education, scientific inquiry, academic research, and any advertising or other promotional activity including written, oral and electronic communication of any form, associated with the disclosure. Additionally, the kits may include other components depending on the specific application, as described herein.

The kits may contain any one or more of the components described herein in one or more containers. The kits may have a variety of forms, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The kits may also include other components, depending on the specific application, for example, containers, cell media, salts, buffers, reagents, etc.

### Additional Embodiments

It should be understood that any combination of enzymes expressed in Table 2 or Table 3 may be applied to the pathways in Table 1. Exemplary, non-limiting enzyme combinations for use in the synthesis of allulose as described by the methods herein are provided below and should be understood to optionally include fewer enzymes (e.g., A1, I1, M1, E1, P1 may be without one or more than one enzyme in the combination), may be optionally modified to replace an α-glucan phosphorylase for any cellodextrin phosphorylase described herein, or may optionally include a debranching enzyme as detailed in Table 3: (A1,I1,M1,E1,P1);(A1,I1,M1,E1,P2);(A1,I1,M1,E1,P3);(A1,I1,M1,E1,P4);(A1,I1,M1,E1,P5);(A1,I1,M1,E1,P6);(A1,I1,M1,E1,P7);(A1,I1,M1,E1,P8);(A1,I1,M1,E1,P9);(A1,I1,M1,E1,P10);(A1,I1,M1,E1,P11);(A1,I1,M1,E1,P12);(A1,I1,M1,E1,P13);(A1,I1,M1,E1,P14);(A1,I1,M1,E1,P15); (A1,I1,M1,E1,P16);(A1,I1,M1,E1,P17);(A1,I1,M1,E1,P18);(A1,I1,M1,E1,P19);(A1,I1,M1,E1,P20);(A1,I1,M1,E2,P1);(A1,I1,M1,E2,P2);(A1,I1,M1,E2,P3);(A1,I1,M1,E2,P4);(A1,I1,M1,E2,P5);(A1,I1,M1,E2,P6);(A1,I1,M1,E2,P7);(A1,I1,M1,E2,P8);(A1,I1,M1,E2,P9);(A1,I1,M1,E2,P10); (A1,I1,M1,E2,P11);(A1,I1,M1,E2,P12);(A1,I1,M1,E2,P13);(A1,I1,M1,E2,P14);(A1,I1,M1,E2,P15);(A1,I1,M1,E2,P16);(A1,I1,M1,E2,P17);(A1,I1,M1,E2,P18);(A1,I1,M1,E2,P19);(A1,I1,M1,E2,P20);(A1,I1,M1,E3,P1);(A1,I1,M1,E3,P2);(A1,I1,M1,E3,P3);(A1,I1,M1,E3,P4);(A1,I1,M1,E3,P5);(A1,I1,M1,E3,P6);(A1,I1,M1,E3,P7);(A1,I1,M1,E3,P8);(A1,I1,M1,E3,P9);(A1,I1,M1,E3,P10);(A1,I1,M1,E3,P11);(A1,I1,M1,E3,P12);(A1,I1,M1,E3,P13);(A1,I1,M1,E3,P14);(A1,I1,M1,E3,P15);(A1,I1,M1,E3,P16);(A1,I1,M1,E3,P17);(A1,I1,M1,E3,P18);(A1,I1,M1,E3,P19);(A1,I1,M1,E3,P20);(A1,I1,M1,E4,P1);(A1,I1,M1,E4,P2);(A1,I1,M1,E4,P3);(A1,I1,M1,E4,P4);(A1,I1,M1,E4,P5);(A1,I1,M1,E4,P6);(A1,I1,M1,E4,P7);(A1,I1,M1,E4,P8);(A1,I1,M1,E4,P9);(A1,I1,M1,E4,P10);(A1,I1,M1,E4,P11);(A1,I1,M1,E4,P12);(A1,I1,M1,E4,P13);(A1,I1,M1,E4,P14);(A1,I1,M1,E4,P15);(A1,I1,M1,E4,P16);(A1,I1,M1,E4,P17);(A1,I1,M1,E4,P18);(A1,I1,M1,E4,P19);(A1,I1,M1,E4,P20);(A1,I1,M1,E5,P1);(A1,I1,M1,E5,P2);(A1,I1,M1,E5,P3);(A1,I1,M1,E5,P4);(A1,I1,M1,E5,P5);(A1,I1,M1,E5,P6);(A1,I1,M1,E5,P7);(A1,I1,M1,E5,P8);(A1,I1,M1,E5,P9);(A1,I1,M1,E5,P10);(A1,I1,M1,E5,P11);(A1,I1,M1,E5,P12);(A1,I1,M1,E5,P13);(A1,I1,M1,E5,P14);(A1,I1,M1,E5,P15);(A1,I1,M1,E5,P16);(A1,I1,M1,E5,P17);(A1,I1,M1,E5,P18);(A1,I1,M1,E5,P19);(A1,I1,M1,E5,P20);(A1,I1,M1,E6,P1);(A1,I1,M1,E6,P2);(A1,I1,M1,E6,P3);(A1,I1,M1,E6,P4);(A1,I1,M1,E6,P5);(A1,I1,M1,E6,P6);(A1,I1,M1,E6,P7);(A1,I1,M1,E6,P8);(A1,I1,M1,E6,P9);(A1,I1,M1,E6,P10);(A1,I1,M1,E6,P11);(A1,I1,M1,E6,P12);(A1,I1,M1,E6,P13);(A1,I1,M1,E6,P14);(A1,I1,M1,E6,P15);(A1,I1,M1,E6,P16);(A1,I1,M1,E6,P17);(A1,I1,M1,E6,P18);(A1,I1,M1,E6,P19);(A1,I1,M1,E6,P20);(A1,I1,M1,E7,P1);(A1,I1,M1,E7,P2);(A1,I1,M1,E7,P3);(A1,I1,M1,E7,P4);(A1,I1,M1,E7,P5);(A1,I1,M1,E7,P6);(A1,I1,M1,E7,P7);(A1,I1,M1,E7,P8);(A1,I1,M1,E7,P9);(A1,I1,M1,E7,P10);(A1,I1,M1,E7,P11);(A1,I1,M1,E7,P12);(A1,I1,M1,E7,P13);(A1,I1,M1,E7,P14);(A1,I1,M1,E7,P15);(A1,I1,M1,E7,P16);(A1,I1,M1,E7,P17);(A1,I1,M1,E7,P18);(A1,I1,M1,E7,P19);(A1,I1,M1,E7,P20);(A1,I1,M1,E8,P1);(A1,I1,M1,E8,P2);(A1,I1,M1,E8,P3);(A1,I1,M1,E8,P4);(A1,I1,M1,E8,P5);(A1,I1,M1,E8,P6);(A1,I1,M1,E8,P7);(A1,I1,M1,E8,P8);(A1,I1,M1,E8,P9);(A1,I1,M1,E8,P10);(A1,I1,M1,E8,P11);(A1,I1,M1,E8,P12);(A1,I1,M1,E8,P13);(A1,I1,M1,E8,P14);(A1,I1,M1,E8,P15);(A1,I1,M1,E8,P16);(A1,I1,M1,E8,P17);(A1,I1,M1,E8,P18);(A1,I1,M1,E8,P19);(A1,I1,M1,E8,P20);(A1,I1,M1,E9,P1);(A1,I1,M1,E9,P2);(A1,I1,M1,E9,P3);(A1,I1,M1,E9,P4);(A1,I1,M1,E9,P5);(A1,I1,M1,E9,P6);(A1,I1,M1,E9,P7);(A1,I1,M1,E9,P8);(A1,I1,M1,E9,P9);(A1,I1,M1,E9,P10);(A1,I1,M1,E9,P11);(A1,I1,M1,E9,P12);(A1,I1,M1,E9,P13);(A1,I1,M1,E9,P14);(A1,I1,M1,E9,P15);(A1,I1,M1,E9,P16);(A1,I1,M1,E9,P17);(A1,I1,M1,E9,P18);(A1,I1,M1,E9,P19);(A1,I1,M1,E9,P20);(A1,I1,M2,E1,P1);(A1,I1,M2,E1,P2);(A1,I1,M2,E1,P3);(A1,I1,M2,E1,P4);(A1,I1,M2,E1,P5);(A1,I1,M2,E1,P6);(A1,I1,M2,E1,P7);(A1,I1,M2,E1,P8);(A1,I1,M2,E1,P9);(A1,I1,M2,E1,P10);(A1,I1,M2,E1,P11);(A1,I1,M2,E1,P12);(A1,I1,M2,E1,P13);(A1,I1,M2,E1,P14);(A1,I1,M2,E1,P15);(A1,I1,M2,E1,P16);(A1,I1,M2,E1,P17);(A1,I1,M2,E1,P18);(A1,I1,M2,E1,P19);(A1,I1,M2,E1,P20);(A1,I1,M2,E2,P1);(A1,I1,M2,E2,P2);(A1,I1,M2,E2,P3);(A1,I1,M2,E2,P4);(A1,I1,M2,E2,P5);(A1,I1,M2,E2,P6);(A1,I1,M2,E2,P7);(A1,I1,M2,E2,P8);(A1,I1,M2,E2,P9);(A1,I1,M2,E2,P10);(A1,I1,M2,E2,P11);(A1,I1,M2,E2,P12);(A1,I1,M2,E2,P13);(A1,I1,M2,E2,P14);(A1,I1,M2,E2,P15);(A1,I1,M2,E2,P16);(A1,I1,M2,E2,P17);(A1,I1,M2,E2,P18);(A1,I1,M2,E2,P19);(A1,I1,M2,E2,P20);(A1,I1,M2,E3,P1);(A1,I1,M2,E3,P2);(A1,I1,M2,E3,P3);(A1,I1,M2,E3,P4);(A1,I1,M2,E3,P5);(A1,I1,M2,E3,P6);(A1,I1,M2,E3,P7);(A1,I1,M2,E3,P8);(A1,I1,M2,E3,P9);(A1,I1,M2,E3,P10);(A1,I1,M2,E3,P11);(A1,I1,M2,E3,P12);(A1,I1,M2,E3,P13);(A1,I1,M2,E3,P14);(A1,I1,M2,E3,P15);(A1,I1,M2,E3,P16);(A1,I1,M2,E3,P17);(A1,I1,M2,E3,P18);(A1,I1,M2,E3,P19);(A1,I1,M2,E3,P20);(A1,I1,M2,E4,P1);(A1,I1,M2,E4,P2);(A1,I1,M2,E4,P3);(A1,I1,M2,E4,P4);(A1,I1,M2,E4,P5);(A1,I1,M2,E4,P6);(A1,I1,M2,E4,P7);(A1,I1,M2,E4,P8);(A1,I1,M2,E4,P9);(A1,I1,M2,E4,P10);(A1,I1,M2,E4,P11);(A1,I1,M2,E4,P12);(A1,I1,M2,E4,P13);(A1,I1,M2,E4,P14);(A1,I1,M2,E4,P15);(A1,I1,M2,E4,P16);(A1,I1,M2,E4,P17);(A1,I1,M2,E4,P18);(A1,I1,M2,E4,P19);(A1,I1,M2,E4,P20);(A1,I1,M2,E5,P1);(A1,I1,M2,E5,P2);(A1,I1,M2,E5,P3);(A1,I1,M2,E5,P4);(A1,I1,M2,E5,P5);(A1,I1,M2,E5,P6);(A1,I1,M2,E5,P7);(A1,I1,M2,E5,P8);(A1,I1,M2,E5,P9);(A1,I1,M2,E5,P10);(A1,I1,M2,E5,P11);(A1,I1,M2,E5,P12);(A1,I1,M2,E5,P13);(A1,I1,M2,E5,P14);(A1,I1,M2,E5,P15);(A1,I1,M2,E5,P16);(A1,I1,M2,E5,P17);(A1,I1,M2,E5,P18);(A1,I1,M2,E5,P19);(A1,I1,M2,E5,P20);(A1,I1,M2,E6,P1);(A1,I1,M2,E6,P2);(A1,I1,M2,E6,P3);(A1,I1,M2,E6,P4);(A1,I1,M2,E6,P5);(A1,I1,M2,E6,P6);(A1,I1,M2,E6,P7);(A1,I1,M2,E6,P8);(A1,I1,M2,E6,P9);(A1,I1,M2,E6,P10);(A1,I1,M2,E6,P11);(A1,I1,M2,E6,P12);(A1,I1,M2,E6,P13);(A1,I1,M2,E6,P14);(A1,I1,M2,E6,P15);(A1,I1,M2,E6,P16);(A1,I1,M2,E6,P17);(A1,I1,M2,E6,P18);(A1,I1,M2,E6,P19);(A1,I1,M2,E6,P20);(A1,I1,M2,E7,P1);(A1,I1,M2,E7,P2);(A1,I1,M2,E7,P3);(A1,I1,M2,E7,P4);(A1,I1,M2,E7,P5);(A1,I1,M2,E7,P6);(A1,I1,M2,E7,P7);(A1,I1,M2,E7,P8);(A1,I1,M2,E7,P9);(A1,I1,M2,E7,P10);(A1,I1,M2,E7,P11);(A1,I1,M2,E7,P12);(A1,I1,M2,E7,P13);(A1,I1,M2,E7,P14);(A1,I1,M2,E7,P15);(A1,I1,M2,E7,P16);(A1,I1,M2,E7,P17);(A1,I1,M2,E7,P18);(A1,I1,M2,E7,P19);(A1,I1,M2,E7,P20);(A1,I1,M2,E8,P1);(A1,I1,M2,E8,P2);(A1,I1,M2,E8,P3);(A1,I1,M2,E8,P4);(A1,I1,M2,E8,P5);(A1,I1,M2,E8,P6);(A1,I1,M2,E8,P7);(A1,I1,M2,E8,P8);(A1,I1,M2,E8,P9);(A1,I1,M2,E8,P10);(A1,I1,M2,E8,P11);(A1,I1,M2,E8,P12);(A1,I1,M2,E8,P13);(A1,I1,M2,E8,P14);(A1,I1,M2,E8,P15);(A1,I1,M2,E8,P16);(A1,I1,M2,E8,P17);(A1,I1,M2,E8,P18);(A1,I1,M2,E8,P19);(A1,I1,M2,E8,P20);(A1,I1,M2,E9,P1);(A1,I1,M2,E9,P2);(A1,I1,M2,E9,P3);(A1,I1,M2,E9,P4);(A1,I1,M2,E9,P5);(A1,I1,M2,E9,P6);(A1,I1,M2,E9,P7);(A1,I1,M2,E9,P8);(A1,I1,M2,E9,P9);(A1,I1,M2,E9,P10);(A1,I1,M2,E9,P11);(A1,I1,M2,E9,P12);(A1,I1,M2,E9,P13);(A1,I1,M2,E9,P14);(A1,I1,M2,E9,P15);(A1,I1,M2,E9,P16);(A1,I1,M2,E9,P17);(A1,I1,M2,E9,P18);(A1,I1,M2,E9,P19);(A1,I1,M2,E9,P20);(A1,I1,M3,E1,P1);(A1,I1,M3,E1,P2);(A1,I1,M3,E1,P3);(A1,I1,M3,E1,P4);(A1,I1,M3,E1,P5);(A1,I1,M3,E1,P6);(A1,I1,M3,E1,P7);(A1,I1,M3,E1,P8);(A1,I1,M3,E1,P9);(A1,I1,M3,E1,P10);(A1,I1,M3,E1,P11);(A1,I1,M3,E1,P12);(A1,I1,M3,E1,P13);(A1,I1,M3,E1,P14);(A1,I1,M3,E1,P15);(A1,I1,M3,E1,P16);(A1,I1,M3,E1,P17);(A1,I1,M3,E1,P18);(A1,I1,M3,E1,P19);(A1,I1,M3,E1,P20);(A1,I1,M3,E2,P1);(A1,I1,M3,E2,P2);(A1,I1,M3,E2,P3);(A1,I1,M3,E2,P4);(A1,I1,M3,E2,P5);(A1,I1,M3,E2,P6);(A1,I1,M3,E2,P7);(A1,I1,M3,E2,P8);(A1,I1,M3,E2,P9);(A1,I1,M3,E2,P10);(A1,I1,M3,E2,P11);(A1,I1,M3,E2,P12);(A1,I1,M3,E2,P13);(A1,I1,M3,E2,P14);(A1,I1,M3,E2,P15);(A1,I1,M3,E2,P16);(A1,I1,M3,E2,P17);(A1,I1,M3,E2,P18);(A1,I1,M3,E2,P19);(A1,I1,M3,E2,P20);(A1,I1,M3,E3,P1);(A1,I1,M3,E3,P2);(A1,I1,M3,E3,P3);(A1,I1,M3,E3,P4);(A1,I1,M3,E3,P5);(A1,I1,M3,E3,P6);(A1,I1,M3,E3,P7);(A1,I1,M3,E3,P8);(A1,I1,M3,E3,P9);(A1,I1,M3,E3,P10);(A1,I1,M3,E3,P11);(A1,I1,M3,E3,P12);(A1,I1,M3,E3,P13);(A1,I1,M3,E3,P14);(A1,I1,M3,E3,P15);(A1,I1,M3,E3,P16);(A1,I1,M3,E3,P17);(A1,I1,M3,E3,P18);(A1,I1,M3,E3,P19);(A1,I1,M3,E3,P20);(A1,I1,M3,E4,P1);(A1,I1,M3,E4,P2);(A1,I1,M3,E4,P3);(A1,I1,M3,E4,P4);(A1,I1,M3,E4,P5);(A1,I1,M3,E4,P6);(A1,I1,M3,E4,P7);(A1,I1,M3,E4,P8);(A1,I1,M3,E4,P9);(A1,I1,M3,E4,P10);(A1,I1,M3,E4,P11);(A1,I1,M3,E4,P12);(A1,I1,M3,E4,P13);(A1,I1,M3,E4,P14);(A1,I1,M3,E4,P15);(A1,I1,M3,E4,P16);(A1,I1,M3,E4,P17);(A1,I1,M3,E4,P18);(A1,I1,M3,E4,P19);(A1,I1,M3,E4,P20);(A1,I1,M3,E5,P1);(A1,I1,M3,E5,P2);(A1,I1,M3,E5,P3);(A1,I1,M3,E5,P4);(A1,I1,M3,E5,P5);(A1,I1,M3,E5,P6);(A1,I1,M3,E5,P7);(A1,I1,M3,E5,P8);(A1,I1,M3,E5,P9);(A1,I1,M3,E5,P10);(A1,I1,M3,E5,P11);(A1,I1,M3,E5,P12);(A1,I1,M3,E5,P13);(A1,I1,M3,E5,P14);(A1,I1,M3,E5,P15);(A1,I1,M3,E5,P16);(A1,I1,M3,E5,P17);(A1,I1,M3,E5,P18);(A1,I1,M3,E5,P19);(A1,I1,M3,E5,P20);(A1,I1,M3,E6,P1);(A1,I1,M3,E6,P2);(A1,I1,M3,E6,P3);(A1,I1,M3,E6,P4);(A1,I1,M3,E6,P5);(A1,I1,M3,E6,P6);(A1,I1,M3,E6,P7);(A1,I1,M3,E6,P8);(A1,I1,M3,E6,P9);(A1,I1,M3,E6,P10);(A1,I1,M3,E6,P11);(A1,I1,M3,E6,P12);(A1,I1,M3,E6,P13);(A1,I1,M3,E6,P14);(A1,I1,M3,E6,P15);(A1,I1,M3,E6,P16);(A1,I1,M3,E6,P17);(A1,I1,M3,E6,P18);(A1,I1,M3,E6,P19);(A1,I1,M3,E6,P20);(A1,I1,M3,E7,P1);(A1,I1,M3,E7,P2);(A1,I1,M3,E7,P3);(A1,I1,M3,E7,P4);(A1,I1,M3,E7,P5);(A1,I1,M3,E7,P6);(A1,I1,M3,E7,P7);(A1,I1,M3,E7,P8);(A1,I1,M3,E7,P9);(A1,I1,M3,E7,P10);(A1,I1,M3,E7,P11);(A1,I1,M3,E7,P12);(A1,I1,M3,E7,P13);(A1,I1,M3,E7,P14);(A1,I1,M3,E7,P15);(A1,I1,M3,E7,P16);(A1,I1,M3,E7,P17);(A1,I1,M3,E7,P18);(A1,I1,M3,E7,P19);(A1,I1,M3,E7,P20);(A1,I1,M3,E8,P1);(A1,I1,M3,E8,P2);(A1,I1,M3,E8,P3);(A1,I1,M3,E8,P4);(A1,I1,M3,E8,P5);(A1,I1,M3,E8,P6);(A1,I1,M3,E8,P7);(A1,I1,M3,E8,P8);(A1,I1,M3,E8,P9);(A1,I1,M3,E8,P10);(A1,I1,M3,E8,P11);(A1,I1,M3,E8,P12);(A1,I1,M3,E8,P13);(A1,I1,M3,E8,P14);(A1,I1,M3,E8,P15);(A1,I1,M3,E8,P16);(A1,I1,M3,E8,P17);(A1,I1,M3,E8,P18);(A1,I1,M3,E8,P19);(A1,I1,M3,E8,P20);(A1,I1,M3,E9,P1);(A1,I1,M3,E9,P2);(A1,I1,M3,E9,P3);(A1,I1,M3,E9,P4);(A1,I1,M3,E9,P5);(A1,I1,M3,E9,P6);(A1,I1,M3,E9,P7);(A1,I1,M3,E9,P8);(A1,I1,M3,E9,P9);(A1,I1,M3,E9,P10);(A1,I1,M3,E9,P11);(A1,I1,M3,E9,P12);(A1,I1,M3,E9,P13);(A1,I1,M3,E9,P14);(A1,I1,M3,E9,P15);(A1,I1,M3,E9,P16);(A1,I1,M3,E9,P17);(A1,I1,M3,E9,P18);(A1,I1,M3,E9,P19);(A1,I1,M3,E9,P20);(A1,I1,M4,E1,P1);(A1,I1,M4,E1,P2);(A1,I1,M4,E1,P3);(A1,I1,M4,E1,P4);(A1,I1,M4,E1,P5);(A1,I1,M4,E1,P6);(A1,I1,M4,E1,P7);(A1,I1,M4,E1,P8);(A1,I1,M4,E1,P9);(A1,I1,M4,E1,P10);(A1,I1,M4,E1,P11);(A1,I1,M4,E1,P12);(A1,I1,M4,E1,P13);(A1,I1,M4,E1,P14);(A1,I1,M4,E1,P15);(A1,I1,M4,E1,P16);(A1,I1,M4,E1,P17);(A1,I1,M4,E1,P18);(A1,I1,M4,E1,P19);(A1,I1,M4,E1,P20);(A1,I1,M4,E2,P1);(A1,I1,M4,E2,P2);(A1,I1,M4,E2,P3);(A1,I1,M4,E2,P4);(A1,I1,M4,E2,P5);(A1,I1,M4,E2,P6);(A1,I1,M4,E2,P7);(A1,I1,M4,E2,P8);(A1,I1,M4,E2,P9);(A1,I1,M4,E2,P10);(A1,I1,M4,E2,P11);(A1,I1,M4,E2,P12);(A1,I1,M4,E2,P13);(A1,I1,M4,E2,P14);(A1,I1,M4,E2,P15);(A1,I1,M4,E2,P16);(A1,I1,M4,E2,P17);(A1,I1,M4,E2,P18);(A1,I1,M4,E2,P19);(A1,I1,M4,E2,P20);(A1,I1,M4,E3,P1);(A1,I1,M4,E3,P2);(A1,I1,M4,E3,P3);(A1,I1,M4,E3,P4);(A1,I1,M4,E3,P5);(A1,I1,M4,E3,P6);(A1,I1,M4,E3,P7);(A1,I1,M4,E3,P8);(A1,I1,M4,E3,P9);(A1,I1,M4,E3,P10);(A1,I1,M4,E3,P11);(A1,I1,M4,E3,P12);(A1,I1,M4,E3,P13);(A1,I1,M4,E3,P14);(A1,I1,M4,E3,P15);(A1,I1,M4,E3,P16);(A1,I1,M4,E3,P17);(A1,I1,M4,E3,P18);(A1,I1,M4,E3,P19);(A1,I1,M4,E3,P20);(A1,I1,M4,E4,P1);(A1,I1,M4,E4,P2);(A1,I1,M4,E4,P3);(A1,I1,M4,E4,P4);(A1,I1,M4,E4,P5);(A1,I1,M4,E4,P6);(A1,I1,M4,E4,P7);(A1,I1,M4,E4,P8);(A1,I1,M4,E4,P9);(A1,I1,M4,E4,P10);(A1,I1,M4,E4,P11);(A1,I1,M4,E4,P12);(A1,I1,M4,E4,P13);(A1,I1,M4,E4,P14);(A1,I1,M4,E4,P15);(A1,I1,M4,E4,P16);(A1,I1,M4,E4,P17);(A1,I1,M4,E4,P18);(A1,I1,M4,E4,P19);(A1,I1,M4,E4,P20);(A1,I1,M4,E5,P1);(A1,I1,M4,E5,P2);(A1,I1,M4,E5,P3);(A1,I1,M4,E5,P4);(A1,I1,M4,E5,P5);(A1,I1,M4,E5,P6);(A1,I1,M4,E5,P7);(A1,I1,M4,E5,P8);(A1,I1,M4,E5,P9);(A1,I1,M4,E5,P10);(A1,I1,M4,E5,P11);(A1,I1,M4,E5,P12);(A1,I1,M4,E5,P13);(A1,I1,M4,E5,P14);(A1,I1,M4,E5,P15);(A1,I1,M4,E5,P16);(A1,I1,M4,E5,P17);(A1,I1,M4,E5,P18);(A1,I1,M4,E5,P19);(A1,I1,M4,E5,P20);(A1,I1,M4,E6,P1);(A1,I1,M4,E6,P2);(A1,I1,M4,E6,P3);(A1,I1,M4,E6,P4);(A1,I1,M4,E6,P5);(A1,I1,M4,E6,P6);(A1,I1,M4,E6,P7);(A1,I1,M4,E6,P8);(A1,I1,M4,E6,P9);(A1,I1,M4,E6,P10);(A1,I1,M4,E6,P11);(A1,I1,M4,E6,P12);(A1,I1,M4,E6,P13);(A1,I1,M4,E6,P14);(A1,I1,M4,E6,P15);(A1,I1,M4,E6,P16);(A1,I1,M4,E6,P17);(A1,I1,M4,E6,P18);(A1,I1,M4,E6,P19);(A1,I1,M4,E6,P20);(A1,I1,M4,E7,P1);(A1,I1,M4,E7,P2);(A1,I1,M4,E7,P3);(A1,I1,M4,E7,P4);(A1,I1,M4,E7,P5);(A1,I1,M4,E7,P6);(A1,I1,M4,E7,P7);(A1,I1,M4,E7,P8);(A1,I1,M4,E7,P9);(A1,I1,M4,E7,P10);(A1,I1,M4,E7,P11);(A1,I1,M4,E7,P12);(A1,I1,M4,E7,P13);(A1,I1,M4,E7,P14);(A1,I1,M4,E7,P15);(A1,I1,M4,E7,P16);(A1,I1,M4,E7,P17);(A1,I1,M4,E7,P18);(A1,I1,M4,E7,P19);(A1,I1,M4,E7,P20);(A1,I1,M4,E8,P1);(A1,I1,M4,E8,P2);(A1,I1,M4,E8,P3);(A1,I1,M4,E8,P4);(A1,I1,M4,E8,P5);(A1,I1,M4,E8,P6);(A1,I1,M4,E8,P7);(A1,I1,M4,E8,P8);(A1,I1,M4,E8,P9);(A1,I1,M4,E8,P10);(A1,I1,M4,E8,P11);(A1,I1,M4,E8,P12);(A1,I1,M4,E8,P13);(A1,I1,M4,E8,P14);(A1,I1,M4,E8,P15);(A1,I1,M4,E8,P16);(A1,I1,M4,E8,P17);(A1,I1,M4,E8,P18);(A1,I1,M4,E8,P19);(A1,I1,M4,E8,P20);(A1,I1,M4,E9,P1);(A1,I1,M4,E9,P2);(A1,I1,M4,E9,P3);(A1,I1,M4,E9,P4);(A1,I1,M4,E9,P5);(A1,I1,M4,E9,P6);(A1,I1,M4,E9,P7);(A1,I1,M4,E9,P8);(A1,I1,M4,E9,P9);(A1,I1,M4,E9,P10);(A1,I1,M4,E9,P11);(A1,I1,M4,E9,P12);(A1,I1,M4,E9,P13);(A1,I1,M4,E9,P14);(A1,I1,M4,E9,P15);(A1,I1,M4,E9,P16);(A1,I1,M4,E9,P17);(A1,I1,M4,E9,P18);(A1,I1,M4,E9,P19);(A1,I1,M4,E9,P20);(A1,I1,M5,E1,P1);(A1,I1,M5,E1,P2);(A1,I1,M5,E1,P3);(A1,I1,M5,E1,P4);(A1,I1,M5,E1,P5);(A1,I1,M5,E1,P6);(A1,I1,M5,E1,P7);(A1,I1,M5,E1,P8);(A1,I1,M5,E1,P9);(A1,I1,M5,E1,P10);(A1,I1,M5,E1,P11);(A1,I1,M5,E1,P12);(A1,I1,M5,E1,P13);(A1,I1,M5,E1,P14);(A1,I1,M5,E1,P15);(A1,I1,M5,E1,P16);(A1,I1,M5,E1,P17);(A1,I1,M5,E1,P18);(A1,I1,M5,E1,P19);(A1,I1,M5,E1,P20);(A1,I1,M5,E2,P1);(A1,I1,M5,E2,P2);(A1,I1,M5,E2,P3);(A1,I1,M5,E2,P4);(A1,I1,M5,E2,P5);(A1,I1,M5,E2,P6);(A1,I1,M5,E2,P7);(A1,I1,M5,E2,P8);(A1,I1,M5,E2,P9);(A1,I1,M5,E2,P10);(A1,I1,M5,E2,P11);(A1,I1,M5,E2,P12);(A1,I1,M5,E2,P13);(A1,I1,M5,E2,P14);(A1,I1,M5,E2,P15);(A1,I1,M5,E2,P16);(A1,I1,M5,E2,P17);(A1,I1,M5,E2,P18);(A1,I1,M5,E2,P19);(A1,I1,M5,E2,P20);(A1,I1,M5,E3,P1);(A1,I1,M5,E3,P2);(A1,I1,M5,E3,P3);(A1,I1,M5,E3,P4);(A1,I1,M5,E3,P5);(A1,I1,M5,E3,P6);(A1,I1,M5,E3,P7);(A1,I1,M5,E3,P8);(A1,I1,M5,E3,P9);(A1,I1,M5,E3,P10);(A1,I1,M5,E3,P11);(A1,I1,M5,E3,P12);(A1,I1,M5,E3,P13);(A1,I1,M5,E3,P14);(A1,I1,M5,E3,P15);(A1,I1,M5,E3,P16);(A1,I1,M5,E3,P17);(A1,I1,M5,E3,P18);(A1,I1,M5,E3,P19);(A1,I1,M5,E3,P20);(A1,I1,M5,E4,P1);(A1,I1,M5,E4,P2);(A1,I1,M5,E4,P3);(A1,I1,M5,E4,P4);(A1,I1,M5,E4,P5);(A1,I1,M5,E4,P6);(A1,I1,M5,E4,P7);(A1,I1,M5,E4,P8);(A1,I1,M5,E4,P9);(A1,I1,M5,E4,P10);(A1,I1,M5,E4,P11);(A1,I1,M5,E4,P12);(A1,I1,M5,E4,P13);(A1,I1,M5,E4,P14);(A1,I1,M5,E4,P15);(A1,I1,M5,E4,P16);(A1,I1,M5,E4,P17);(A1,I1,M5,E4,P18);(A1,I1,M5,E4,P19);(A1,I1,M5,E4,P20);(A1,I1,M5,E5,P1);(A1,I1,M5,E5,P2);(A1,I1,M5,E5,P3);(A1,I1,M5,E5,P4);(A1,I1,M5,E5,P5);(A1,I1,M5,E5,P6);(A1,I1,M5,E5,P7);(A1,I1,M5,E5,P8);(A1,I1,M5,E5,P9);(A1,I1,M5,E5,P10);(A1,I1,M5,E5,P11);(A1,I1,M5,E5,P12);(A1,I1,M5,E5,P13);(A1,I1,M5,E5,P14);(A1,I1,M5,E5,P15);(A1,I1,M5,E5,P16);(A1,I1,M5,E5,P17);(A1,I1,M5,E5,P18);(A1,I1,M5,E5,P19);(A1,I1,M5,E5,P20);(A1,I1,M5,E6,P1);(A1,I1,M5,E6,P2);(A1,I1,M5,E6,P3);(A1,I1,M5,E6,P4);(A1,I1,M5,E6,P5);(A1,I1,M5,E6,P6);(A1,I1,M5,E6,P7);(A1,I1,M5,E6,P8);(A1,I1,M5,E6,P9);(A1,I1,M5,E6,P10);(A1,I1,M5,E6,P11);(A1,I1,M5,E6,P12);(A1,I1,M5,E6,P13);(A1,I1,M5,E6,P14);(A1,I1,M5,E6,P15);(A1,I1,M5,E6,P16);(A1,I1,M5,E6,P17);(A1,I1,M5,E6,P18);(A1,I1,M5,E6,P19);(A1,I1,M5,E6,P20);(A1,I1,M5,E7,P1);(A1,I1,M5,E7,P2);(A1,I1,M5,E7,P3);(A1,I1,M5,E7,P4);(A1,I1,M5,E7,P5);(A1,I1,M5,E7,P6);(A1,I1,M5,E7,P7);(A1,I1,M5,E7,P8);(A1,I1,M5,E7,P9);(A1,I1,M5,E7,P10);(A1,I1,M5,E7,P11);(A1,I1,M5,E7,P12);(A1,I1,M5,E7,P13);(A1,I1,M5,E7,P14);(A1,I1,M5,E7,P15);(A1,I1,M5,E7,P16);(A1,I1,M5,E7,P17);(A1,I1,M5,E7,P18);(A1,I1,M5,E7,P19);(A1,I1,M5,E7,P20);(A1,I1,M5,E8,P1);(A1,I1,M5,E8,P2);(A1,I1,M5,E8,P3);(A1,I1,M5,E8,P4);(A1,I1,M5,E8,P5);(A1,I1,M5,E8,P6);(A1,I1,M5,E8,P7);(A1,I1,M5,E8,P8);(A1,I1,M5,E8,P9);(A1,I1,M5,E8,P10);(A1,I1,M5,E8,P11);(A1,I1,M5,E8,P12);(A1,I1,M5,E8,P13);(A1,I1,M5,E8,P14);(A1,I1,M5,E8,P15);(A1,I1,M5,E8,P16);(A1,I1,M5,E8,P17);(A1,I1,M5,E8,P18);(A1,I1,M5,E8,P19);(A1,I1,M5,E8,P20);(A1,I1,M5,E9,P1);(A1,I1,M5,E9,P2);(A1,I1,M5,E9,P3);(A1,I1,M5,E9,P4);(A1,I1,M5,E9,P5);(A1,I1,M5,E9,P6);(A1,I1,M5,E9,P7);(A1,I1,M5,E9,P8);(A1,I1,M5,E9,P9);(A1,I1,M5,E9,P10);(A1,I1,M5,E9,P11);(A1,I1,M5,E9,P12);(A1,I1,M5,E9,P13);(A1,I1,M5,E9,P14);(A1,I1,M5,E9,P15);(A1,I1,M5,E9,P16);(A1,I1,M5,E9,P17);(A1,I1,M5,E9,P18);(A1,I1,M5,E9,P19);(A1,I1,M5,E9,P20);(A1,I1,M6,E1,P1);(A1,I1,M6,E1,P2);(A1,I1,M6,E1,P3);(A1,I1,M6,E1,P4);(A1,I1,M6,E1,P5);(A1,I1,M6,E1,P6);(A1,I1,M6,E1,P7);(A1,I1,M6,E1,P8);(A1,I1,M6,E1,P9);(A1,I1,M6,E1,P10);(A1,I1,M6,E1,P11);(A1,I1,M6,E1,P12);(A1,I1,M6,E1,P13);(A1,I1,M6,E1,P14);(A1,I1,M6,E1,P15);(A1,I1,M6,E1,P16);(A1,I1,M6,E1,P17);(A1,I1,M6,E1,P18);(A1,I1,M6,E1,P19);(A1,I1,M6,E1,P20);(A1,I1,M6,E2,P1);(A1,I1,M6,E2,P2);(A1,I1,M6,E2,P3);(A1,I1,M6,E2,P4);(A1,I1,M6,E2,P5);(A1,I1,M6,E2,P6);(A1,I1,M6,E2,P7);(A1,I1,M6,E2,P8);(A1,I1,M6,E2,P9);(A1,I1,M6,E2,P10);(A1,I1,M6,E2,P11);(A1,I1,M6,E2,P12);(A1,I1,M6,E2,P13);(A1,I1,M6,E2,P14);(A1,I1,M6,E2,P15);(A1,I1,M6,E2,P16);(A1,I1,M6,E2,P17);(A1,I1,M6,E2,P18);(A1,I1,M6,E2,P19);(A1,I1,M6,E2,P20);(A1,I1,M6,E3,P1);(A1,I1,M6,E3,P2);(A1,I1,M6,E3,P3);(A1,I1,M6,E3,P4);(A1,I1,M6,E3,P5);(A1,I1,M6,E3,P6);(A1,I1,M6,E3,P7);(A1,I1,M6,E3,P8);(A1,I1,M6,E3,P9);(A1,I1,M6,E3,P10);(A1,I1,M6,E3,P11);(A1,I1,M6,E3,P12);(A1,I1,M6,E3,P13);(A1,I1,M6,E3,P14);(A1,I1,M6,E3,P15);(A1,I1,M6,E3,P16);(A1,I1,M6,E3,P17);(A1,I1,M6,E3,P18);(A1,I1,M6,E3,P19);(A1,I1,M6,E3,P20);(A1,I1,M6,E4,P1);(A1,I1,M6,E4,P2);(A1,I1,M6,E4,P3);(A1,I1,M6,E4,P4);(A1,I1,M6,E4,P5);(A1,I1,M6,E4,P6);(A1,I1,M6,E4,P7);(A1,I1,M6,E4,P8);(A1,I1,M6,E4,P9);(A1,I1,M6,E4,P10);(A1,I1,M6,E4,P11);(A1,I1,M6,E4,P12);(A1,I1,M6,E4,P13);(A1,I1,M6,E4,P14);(A1,I1,M6,E4,P15);(A1,I1,M6,E4,P16);(A1,I1,M6,E4,P17);(A1,I1,M6,E4,P18);(A1,I1,M6,E4,P19);(A1,I1,M6,E4,P20);(A1,I1,M6,E5,P1);(A1,I1,M6,E5,P2);(A1,I1,M6,E5,P3);(A1,I1,M6,E5,P4);(A1,I1,M6,E5,P5);(A1,I1,M6,E5,P6);(A1,I1,M6,E5,P7);(A1,I1,M6,E5,P8);(A1,I1,M6,E5,P9);(A1,I1,M6,E5,P10);(A1,I1,M6,E5,P11);(A1,I1,M6,E5,P12);(A1,I1,M6,E5,P13);(A1,I1,M6,E5,P14);(A1,I1,M6,E5,P15);(A1,I1,M6,E5,P16);(A1,I1,M6,E5,P17);(A1,I1,M6,E5,P18);(A1,I1,M6,E5,P19);(A1,I1,M6,E5,P20);(A1,I1,M6,E6,P1);(A1,I1,M6,E6,P2);(A1,I1,M6,E6,P3);(A1,I1,M6,E6,P4);(A1,I1,M6,E6,P5);(A1,I1,M6,E6,P6);(A1,I1,M6,E6,P7);(A1,I1,M6,E6,P8);(A1,I1,M6,E6,P9);(A1,I1,M6,E6,P10);(A1,I1,M6,E6,P11);(A1,I1,M6,E6,P12);(A1,I1,M6,E6,P13);(A1,I1,M6,E6,P14);(A1,I1,M6,E6,P15);(A1,I1,M6,E6,P16);(A1,I1,M6,E6,P17);(A1,I1,M6,E6,P18);(A1,I1,M6,E6,P19);(A1,I1,M6,E6,P20);(A1,I1,M6,E7,P1);(A1,I1,M6,E7,P2);(A1,I1,M6,E7,P3);(A1,I1,M6,E7,P4);(A1,I1,M6,E7,P5);(A1,I1,M6,E7,P6);(A1,I1,M6,E7,P7);(A1,I1,M6,E7,P8);(A1,I1,M6,E7,P9);(A1,I1,M6,E7,P10);(A1,I1,M6,E7,P11);(A1,I1,M6,E7,P12);(A1,I1,M6,E7,P13);(A1,I1,M6,E7,P14);(A1,I1,M6,E7,P15);(A1,I1,M6,E7,P16);(A1,I1,M6,E7,P17);(A1,I1,M6,E7,P18);(A1,I1,M6,E7,P19);(A1,I1,M6,E7,P20);(A1,I1,M6,E8,P1);(A1,I1,M6,E8,P2);(A1,I1,M6,E8,P3);(A1,I1,M6,E8,P4);(A1,I1,M6,E8,P5);(A1,I1,M6,E8,P6);(A1,I1,M6,E8,P7);(A1,I1,M6,E8,P8);(A1,I1,M6,E8,P9);(A1,I1,M6,E8,P10);(A1,I1,M6,E8,P11);(A1,I1,M6,E8,P12);(A1,I1,M6,E8,P13);(A1,I1,M6,E8,P14);(A1,I1,M6,E8,P15);(A1,I1,M6,E8,P16);(A1,I1,M6,E8,P17);(A1,I1,M6,E8,P18);(A1,I1,M6,E8,P19);(A1,I1,M6,E8,P20);(A1,I1,M6,E9,P1);(A1,I1,M6,E9,P2);(A1,I1,M6,E9,P3);(A1,I1,M6,E9,P4);(A1,I1,M6,E9,P5);(A1,I1,M6,E9,P6);(A1,I1,M6,E9,P7);(A1,I1,M6,E9,P8);(A1,I1,M6,E9,P9);(A1,I1,M6,E9,P10);(A1,I1,M6,E9,P11);(A1,I1,M6,E9,P12);(A1,I1,M6,E9,P13);(A1,I1,M6,E9,P14);(A1,I1,M6,E9,P15);(A1,I1,M6,E9,P16);(A1,I1,M6,E9,P17);(A1,I1,M6,E9,P18);(A1,I1,M6,E9,P19);(A1,I1,M6,E9,P20);(A1,I2,M1,E1,P1);(A1,I2,M1,E1,P2);(A1,I2,M1,E1,P3);(A1,I2,M1,E1,P4);(A1,I2,M1,E1,P5);(A1,I2,M1,E1,P6);(A1,I2,M1,E1,P7);(A1,I2,M1,E1,P8);(A1,I2,M1,E1,P9);(A1,I2,M1,E1,P10);(A1,I2,M1,E1,P11);(A1,I2,M1,E1,P12);(A1,I2,M1,E1,P13);(A1,I2,M1,E1,P14);(A1,I2,M1,E1,P15);(A1,I2,M1,E1,P16);(A1,I2,M1,E1,P17);(A1,I2,M1,E1,P18);(A1,I2,M1,E1,P19);(A1,I2,M1,E1,P20);(A1,I2,M1,E2,P1);(A1,I2,M1,E2,P2);(A1,I2,M1,E2,P3);(A1,I2,M1,E2,P4);(A1,I2,M1,E2,P5);(A1,I2,M1,E2,P6);(A1,I2,M1,E2,P7);(A1,I2,M1,E2,P8);(A1,I2,M1,E2,P9);(A1,I2,M1,E2,P10);(A1,I2,M1,E2,P11);(A1,I2,M1,E2,P12);(A1,I2,M1,E2,P13);(A1,I2,M1,E2,P14);(A1,I2,M1,E2,P15);(A1,I2,M1,E2,P16);(A1,I2,M1,E2,P17);(A1,I2,M1,E2,P18);(A1,I2,M1,E2,P19);(A1,I2,M1,E2,P20);(A1,I2,M1,E3,P1);(A1,I2,M1,E3,P2);(A1,I2,M1,E3,P3);(A1,I2,M1,E3,P4);(A1,I2,M1,E3,P5);(A1,I2,M1,E3,P6);(A1,I2,M1,E3,P7);(A1,I2,M1,E3,P8);(A1,I2,M1,E3,P9);(A1,I2,M1,E3,P10);(A1,I2,M1,E3,P11);(A1,I2,M1,E3,P12);(A1,I2,M1,E3,P13);(A1,I2,M1,E3,P14);(A1,I2,M1,E3,P15);(A1,I2,M1,E3,P16);(A1,I2,M1,E3,P17);(A1,I2,M1,E3,P18);(A1,I2,M1,E3,P19);(A1,I2,M1,E3,P20);(A1,I2,M1,E4,P1);(A1,I2,M1,E4,P2);(A1,I2,M1,E4,P3);(A1,I2,M1,E4,P4);(A1,I2,M1,E4,P5);(A1,I2,M1,E4,P6);(A1,I2,M1,E4,P7);(A1,I2,M1,E4,P8);(A1,I2,M1,E4,P9);(A1,I2,M1,E4,P10);(A1,I2,M1,E4,P11);(A1,I2,M1,E4,P12);(A1,I2,M1,E4,P13);(A1,I2,M1,E4,P14);(A1,I2,M1,E4,P15);(A1,I2,M1,E4,P16);(A1,I2,M1,E4,P17);(A1,I2,M1,E4,P18);(A1,I2,M1,E4,P19);(A1,I2,M1,E4,P20);(A1,I2,M1,E5,P1);(A1,I2,M1,E5,P2);(A1,I2,M1,E5,P3);(A1,I2,M1,E5,P4);(A1,I2,M1,E5,P5);(A1,I2,M1,E5,P6);(A1,I2,M1,E5,P7);(A1,I2,M1,E5,P8);(A1,I2,M1,E5,P9);(A1,I2,M1,E5,P10);(A1,I2,M1,E5,P11);(A1,I2,M1,E5,P12);(A1,I2,M1,E5,P13);(A1,I2,M1,E5,P14);(A1,I2,M1,E5,P15);(A1,I2,M1,E5,P16);(A1,I2,M1,E5,P17);(A1,I2,M1,E5,P18);(A1,I2,M1,E5,P19);(A1,I2,M1,E5,P20);(A1,I2,M1,E6,P1);(A1,I2,M1,E6,P2);(A1,I2,M1,E6,P3);(A1,I2,M1,E6,P4);(A1,I2,M1,E6,P5);(A1,I2,M1,E6,P6);(A1,I2,M1,E6,P7);(A1,I2,M1,E6,P8);(A1,I2,M1,E6,P9);(A1,I2,M1,E6,P10);(A1,I2,M1,E6,P11);(A1,I2,M1,E6,P12);(A1,I2,M1,E6,P13);(A1,I2,M1,E6,P14);(A1,I2,M1,E6,P15);(A1,I2,M1,E6,P16);(A1,I2,M1,E6,P17);(A1,I2,M1,E6,P18);(A1,I2,M1,E6,P19);(A1,I2,M1,E6,P20);(A1,I2,M1,E7,P1);(A1,I2,M1,E7,P2);(A1,I2,M1,E7,P3);(A1,I2,M1,E7,P4);(A1,I2,M1,E7,P5);(A1,I2,M1,E7,P6);(A1,I2,M1,E7,P7);(A1,I2,M1,E7,P8);(A1,I2,M1,E7,P9);(A1,I2,M1,E7,P10);(A1,I2,M1,E7,P11);(A1,I2,M1,E7,P12);(A1,I2,M1,E7,P13);(A1,I2,M1,E7,P14);(A1,I2,M1,E7,P15);(A1,I2,M1,E7,P16);(A1,I2,M1,E7,P17);(A1,I2,M1,E7,P18);(A1,I2,M1,E7,P19);(A1,I2,M1,E7,P20);(A1,I2,M1,E8,P1);(A1,I2,M1,E8,P2);(A1,I2,M1,E8,P3);(A1,I2,M1,E8,P4);(A1,I2,M1,E8,P5);(A1,I2,M1,E8,P6);(A1,I2,M1,E8,P7);(A1,I2,M1,E8,P8);A1,I2,M1,E8,P9);(A1,I2,M1,E8,P10);(A1,I2,M1,E8,P11);(A1,I2,M1,E8,P12);(A1,I2,M1,E8,P13);(A1,I2,M1,E8,P14);(A1,I2,M1,E8,P15);(A1,I2,M1,E8,P16);(A1,I2,M1,E8,P17);(A1,I2,M1,E8,P18);(A1,I2,M1,E8,P19);(A1,I2,M1,E8,P20);(A1,I2,M1,E9,P1);(A1,I2,M1,E9,P2);(A1,I2,M1,E9,P3);(A1,I2,M1,E9,P4);(A1,I2,M1,E9,P5);(A1,I2,M1,E9,P6);(A1,I2,M1,E9,P7);(A1,I2,M1,E9,P8);(A1,I2,M1,E9,P9);(A1,I2,M1,E9,P10);(A1,I2,M1,E9,P11);(A1,I2,M1,E9,P12);(A1,I2,M1,E9,P13);(A1,I2,M1,E9,P14);(A1,I2,M1,E9,P15);(A1,I2,M1,E9,P16);(A1,I2,M1,E9,P17);(A1,I2,M1,E9,P18);(A1,I2,M1,E9,P19);(A1,I2,M1,E9,P20);(A1,I2,M2,E1,P1);(A1,I2,M2,E1,P2);(A1,I2,M2,E1,P3);(A1,I2,M2,E1,P4);(A1,I2,M2,E1,P5);(A1,I2,M2,E1,P6);(A1,I2,M2,E1,P7);(A1,I2,M2,E1,P8);(A1,I2,M2,E1,P9);(A1,I2,M2,E1,P10);(A1,I2,M2,E1,P11);(A1,I2,M2,E1,P12);(A1,I2,M2,E1,P13);(A1,I2,M2,E1,P14);(A1,I2,M2,E1,P15);(A1,I2,M2,E1,P16);(A1,I2,M2,E1,P17);(A1,I2,M2,E1,P18);(A1,I2,M2,E1,P19);(A1,I2,M2,E1,P20);(A1,I2,M2,E2,P1);(A1,I2,M2,E2,P2);(A1,I2,M2,E2,P3);(A1,I2,M2,E2,P4);(A1,I2,M2,E2,P5);(A1,I2,M2,E2,P6);(A1,I2,M2,E2,P7);(A1,I2,M2,E2,P8);(A1,I2,M2,E2,P9);(A1,I2,M2,E2,P10);(A1,I2,M2,E2,P11);(A1,I2,M2,E2,P12);(A1,I2,M2,E2,P13);(A1,I2,M2,E2,P14);(A1,I2,M2,E2,P15);(A1,I2,M2,E2,P16);(A1,I2,M2,E2,P17);(A1,I2,M2,E2,P18);(A1,I2,M2,E2,P19);(A1,I2,M2,E2,P20);(A1,I2,M2,E3,P1);(A1,I2,M2,E3,P2);(A1,I2,M2,E3,P3);(A1,I2,M2,E3,P4);(A1,I2,M2,E3,P5);(A1,I2,M2,E3,P6);(A1,I2,M2,E3,P7);(A1,I2,M2,E3,P8);(A1,I2,M2,E3,P9);(A1,I2,M2,E3,P10);(A1,I2,M2,E3,P11);(A1,I2,M2,E3,P12);(A1,I2,M2,E3,P13);(A1,I2,M2,E3,P14);(A1,I2,M2,E3,P15);(A1,I2,M2,E3,P16);(A1,I2,M2,E3,P17);(A1,I2,M2,E3,P18);(A1,I2,M2,E3,P19);(A1,I2,M2,E3,P20);(A1,I2,M2,E4,P1);(A1,I2,M2,E4,P2);(A1,I2,M2,E4,P3);(A1,I2,M2,E4,P4);(A1,I2,M2,E4,P5);(A1,I2,M2,E4,P6);(A1,I2,M2,E4,P7);(A1,I2,M2,E4,P8);(A1,I2,M2,E4,P9);(A1,I2,M2,E4,P10);(A1,I2,M2,E4,P11);(A1,I2,M2,E4,P12);(A1,I2,M2,E4,P13);(A1,I2,M2,E4,P14);(A1,I2,M2,E4,P15);(A1,I2,M2,E4,P16);(A1,I2,M2,E4,P17);(A1,I2,M2,E4,P18);(A1,I2,M2,E4,P19);(A1,I2,M2,E4,P20);(A1,I2,M2,E5,P1);(A1,I2,M2,E5,P2);(A1,I2,M2,E5,P3);(A1,I2,M2,E5,P4);(A1,I2,M2,E5,P5);(A1,I2,M2,E5,P6);(A1,I2,M2,E5,P7);(A1,I2,M2,E5,P8);(A1,I2,M2,E5,P9);(A1,I2,M2,E5,P10);(A1,I2,M2,E5,P11);(A1,I2,M2,E5,P12);(A1,I2,M2,E5,P13);(A1,I2,M2,E5,P14);(A1,I2,M2,E5,P15);(A1,I2,M2,E5,P16);(A1,I2,M2,E5,P17);(A1,I2,M2,E5,P18);(A1,I2,M2,E5,P19);(A1,I2,M2,E5,P20);(A1,I2,M2,E6,P1);(A1,I2,M2,E6,P2);(A1,I2,M2,E6,P3);(A1,I2,M2,E6,P4);(A1,I2,M2,E6,P5);(A1,I2,M2,E6,P6);(A1,I2,M2,E6,P7);(A1,I2,M2,E6,P8);(A1,I2,M2,E6,P9);(A1,I2,M2,E6,P10);(A1,I2,M2,E6,P11);(A1,I2,M2,E6,P12);(A1,I2,M2,E6,P13);(A1,I2,M2,E6,P14);(A1,I2,M2,E6,P15);(A1,I2,M2,E6,P16);(A1,I2,M2,E6,P17);(A1,I2,M2,E6,P18);(A1,I2,M2,E6,P19);(A1,I2,M2,E6,P20);(A1,I2,M2,E7,P1);(A1,I2,M2,E7,P2);(A1,I2,M2,E7,P3);(A1,I2,M2,E7,P4);(A1,I2,M2,E7,P5);(A1,I2,M2,E7,P6);(A1,I2,M2,E7,P7);(A1,I2,M2,E7,P8);(A1,I2,M2,E7,P9);(A1,I2,M2,E7,P10);(A1,I2,M2,E7,P11);(A1,I2,M2,E7,P12);(A1,I2,M2,E7,P13);(A1,I2,M2,E7,P14);(A1,I2,M2,E7,P15);(A1,I2,M2,E7,P16);(A1,I2,M2,E7,P17);(A1,I2,M2,E7,P18);(A1,I2,M2,E7,P19);(A1,I2,M2,E7,P20);(A1,I2,M2,E8,P1);(A1,I2,M2,E8,P2);(A1,I2,M2,E8,P3);(A1,I2,M2,E8,P4);(A1,I2,M2,E8,P5);(A1,I2,M2,E8,P6);(A1,I2,M2,E8,P7);(A1,I2,M2,E8,P8);(A1,I2,M2,E8,P9);(A1,I2,M2,E8,P10);(A1,I2,M2,E8,P11);(A1,I2,M2,E8,P12);(A1,I2,M2,E8,P13);(A1,I2,M2,E8,P14);(A1,I2,M2,E8,P15);(A1,I2,M2,E8,P16);(A1,I2,M2,E8,P17);(A1,I2,M2,E8,P18);(A1,I2,M2,E8,P19);(A1,I2,M2,E8,P20);(A1,I2,M2,E9,P1);(A1,I2,M2,E9,P2);(A1,I2,M2,E9,P3);(A1,I2,M2,E9,P4);(A1,I2,M2,E9,P5);(A1,I2,M2,E9,P6);(A1,I2,M2,E9,P7);(A1,I2,M2,E9,P8);(A1,I2,M2,E9,P9);(A1,I2,M2,E9,P10);(A1,I2,M2,E9,P11);(A1,I2,M2,E9,P12);(A1,I2,M2,E9,P13);(A1,I2,M2,E9,P14);(A1,I2,M2,E9,P15);(A1,I2,M2,E9,P16);(A1,I2,M2,E9,P17);(A1,I2,M2,E9,P18);(A1,I2,M2,E9,P19);(A1,I2,M2,E9,P20);(A1,I2,M3,E1,P1);(A1,I2,M3,E1,P2);(A1,I2,M3,E1,P3);(A1,I2,M3,E1,P4);(A1,I2,M3,E1,P5);(A1,I2,M3,E1,P6);(A1,I2,M3,E1,P7);(A1,I2,M3,E1,P8);(A1,I2,M3,E1,P9);(A1,I2,M3,E1,P10);(A1,I2,M3,E1,P11);(A1,I2,M3,E1,P12);(A1,I2,M3,E1,P13);(A1,I2,M3,E1,P14);(A1,I2,M3,E1,P15);(A1,I2,M3,E1,P16);(A1,I2,M3,E1,P17);(A1,I2,M3,E1,P18);(A1,I2,M3,E1,P19);(A1,I2,M3,E1,P20);(A1,I2,M3,E2,P1);(A1,I2,M3,E2,P2);(A1,I2,M3,E2,P3);(A1,I2,M3,E2,P4);(A1,I2,M3,E2,P5);(A1,I2,M3,E2,P6);(A1,I2,M3,E2,P7);(A1,I2,M3,E2,P8);(A1,I2,M3,E2,P9);(A1,I2,M3,E2,P10);(A1,I2,M3,E2,P11);(A1,I2,M3,E2,P12);(A1,I2,M3,E2,P13);(A1,I2,M3,E2,P14);(A1,I2,M3,E2,P15);(A1,I2,M3,E2,P16);(A1,I2,M3,E2,P17);(A1,I2,M3,E2,P18);(A1,I2,M3,E2,P19);(A1,I2,M3,E2,P20);(A1,I2,M3,E3,P1);(A1,I2,M3,E3,P2);(A1,I2,M3,E3,P3);(A1,I2,M3,E3,P4);(A1,I2,M3,E3,P5);(A1,I2,M3,E3,P6);(A1,I2,M3,E3,P7);(A1,I2,M3,E3,P8);(A1,I2,M3,E3,P9);(A1,I2,M3,E3,P10);(A1,I2,M3,E3,P11);(A1,I2,M3,E3,P12);(A1,I2,M3,E3,P13);(A1,I2,M3,E3,P14);(A1,I2,M3,E3,P15);(A1,I2,M3,E3,P16);(A1,I2,M3,E3,P17);(A1,I2,M3,E3,P18);(A1,I2,M3,E3,P19);(A1,I2,M3,E3,P20);(A1,I2,M3,E4,P1);(A1,I2,M3,E4,P2);(A1,I2,M3,E4,P3);(A1,I2,M3,E4,P4);(A1,I2,M3,E4,P5);(A1,I2,M3,E4,P6);(A1,I2,M3,E4,P7);(A1,I2,M3,E4,P8);(A1,I2,M3,E4,P9);(A1,I2,M3,E4,P10);(A1,I2,M3,E4,P11);(A1,I2,M3,E4,P12);(A1,I2,M3,E4,P13);(A1,I2,M3,E4,P14);(A1,I2,M3,E4,P15);(A1,I2,M3,E4,P16);(A1,I2,M3,E4,P17);(A1,I2,M3,E4,P18);(A1,I2,M3,E4,P19);(A1,I2,M3,E4,P20);(A1,I2,M3,E5,P1);(A1,I2,M3,E5,P2);(A1,I2,M3,E5,P3);(A1,I2,M3,E5,P4);(A1,I2,M3,E5,P5);(A1,I2,M3,E5,P6);(A1,I2,M3,E5,P7);(A1,I2,M3,E5,P8);(A1,I2,M3,E5,P9);(A1,I2,M3,E5,P10);(A1,I2,M3,E5,P11);(A1,I2,M3,E5,P12);(A1,I2,M3,E5,P13);(A1,I2,M3,E5,P14);(A1,I2,M3,E5,P15);(A1,I2,M3,E5,P16);(A1,I2,M3,E5,P17);(A1,I2,M3,E5,P18);(A1,I2,M3,E5,P19);(A1,I2,M3,E5,P20);(A1,I2,M3,E6,P1);(A1,I2,M3,E6,P2);(A1,I2,M3,E6,P3);(A1,I2,M3,E6,P4);(A1,I2,M3,E6,P5);(A1,I2,M3,E6,P6);(A1,I2,M3,E6,P7);(A1,I2,M3,E6,P8);(A1,I2,M3,E6,P9);(A1,I2,M3,E6,P10);(A1,I2,M3,E6,P11);(A1,I2,M3,E6,P12);(A1,I2,M3,E6,P13);(A1,I2,M3,E6,P14);(A1,I2,M3,E6,P15);(A1,I2,M3,E6,P16);(A1,I2,M3,E6,P17);(A1,I2,M3,E6,P18);(A1,I2,M3,E6,P19);(A1,I2,M3,E6,P20);(A1,I2,M3,E7,P1);(A1,I2,M3,E7,P2);(A1,I2,M3,E7,P3);(A1,I2,M3,E7,P4);(A1,I2,M3,E7,P5);(A1,I2,M3,E7,P6);(A1,I2,M3,E7,P7);(A1,I2,M3,E7,P8);(A1,I2,M3,E7,P9);(A1,I2,M3,E7,P10);(A1,I2,M3,E7,P11);(A1,I2,M3,E7,P12);(A1,I2,M3,E7,P13);(A1,I2,M3,E7,P14);(A1,I2,M3,E7,P15);(A1,I2,M3,E7,P16);(A1,I2,M3,E7,P17);(A1,I2,M3,E7,P18);(A1,I2,M3,E7,P19);(A1,I2,M3,E7,P20);(A1,I2,M3,E8,P1);(A1,I2,M3,E8,P2);(A1,I2,M3,E8,P3);(A1,I2,M3,E8,P4);(A1,I2,M3,E8,P5);(A1,I2,M3,E8,P6);(A1,I2,M3,E8,P7);(A1,I2,M3,E8,P8);(A1,I2,M3,E8,P9);(A1,I2,M3,E8,P10);(A1,I2,M3,E8,P11);(A1,I2,M3,E8,P12);(A1,I2,M3,E8,P13);(A1,I2,M3,E8,P14);(A1,I2,M3,E8,P15);(A1,I2,M3,E8,P16);(A1,I2,M3,E8,P17);(A1,I2,M3,E8,P18);(A1,I2,M3,E8,P19);(A1,I2,M3,E8,P20);(A1,I2,M3,E9,P1);(A1,I2,M3,E9,P2);(A1,I2,M3,E9,P3);(A1,I2,M3,E9,P4);(A1,I2,M3,E9,P5);(A1,I2,M3,E9,P6);(A1,I2,M3,E9,P7);(A1,I2,M3,E9,P8);(A1,I2,M3,E9,P9);(A1,I2,M3,E9,P10);(A1,I2,M3,E9,P11);(A1,I2,M3,E9,P12);(A1,I2,M3,E9,P13);(A1,I2,M3,E9,P14);(A1,I2,M3,E9,P15);(A1,I2,M3,E9,P16);(A1,I2,M3,E9,P17);(A1,I2,M3,E9,P18);(A1,I2,M3,E9,P19);(A1,I2,M3,E9,P20);(A1,I2,M4,E1,P1);(A1,I2,M4,E1,P2);(A1,I2,M4,E1,P3);(A1,I2,M4,E1,P4);(A1,I2,M4,E1,P5);(A1,I2,M4,E1,P6);(A1,I2,M4,E1,P7);(A1,I2,M4,E1,P8);(A1,I2,M4,E1,P9);(A1,I2,M4,E1,P10);(A1,I2,M4,E1,P11);(A1,I2,M4,E1,P12);(A1,I2,M4,E1,P13);(A1,I2,M4,E1,P14);(A1,I2,M4,E1,P15);(A1,I2,M4,E1,P16);(A1,I2,M4,E1,P17);(A1,I2,M4,E1,P18);(A1,I2,M4,E1,P19);(A1,I2,M4,E1,P20);(A1,I2,M4,E2,P1);(A1,I2,M4,E2,P2);(A1,I2,M4,E2,P3);(A1,I2,M4,E2,P4);(A1,I2,M4,E2,P5);(A1,I2,M4,E2,P6);(A1,I2,M4,E2,P7);(A1,I2,M4,E2,P8);(A1,I2,M4,E2,P9);(A1,I2,M4,E2,P10);(A1,I2,M4,E2,P11);(A1,I2,M4,E2,P12);(A1,I2,M4,E2,P13);(A1,I2,M4,E2,P14);(A1,I2,M4,E2,P15);(A1,I2,M4,E2,P16);(A1,I2,M4,E2,P17);(A1,I2,M4,E2,P18);(A1,I2,M4,E2,P19);(A1,I2,M4,E2,P20);(A1,I2,M4,E3,P1);(A1,I2,M4,E3,P2);(A1,I2,M4,E3,P3);(A1,I2,M4,E3,P4);(A1,I2,M4,E3,P5);(A1,I2,M4,E3,P6);(A1,I2,M4,E3,P7);(A1,I2,M4,E3,P8);(A1,I2,M4,E3,P9);(A1,I2,M4,E3,P10);(A1,I2,M4,E3,P11);(A1,I2,M4,E3,P12);(A1,I2,M4,E3,P13);(A1,I2,M4,E3,P14);(A1,I2,M4,E3,P15);(A1,I2,M4,E3,P16);(A1,I2,M4,E3,P17);(A1,I2,M4,E3,P18);(A1,I2,M4,E3,P19);(A1,I2,M4,E3,P20);(A1,I2,M4,E4,P1);(A1,I2,M4,E4,P2);(A1,I2,M4,E4,P3);(A1,I2,M4,E4,P4);(A1,I2,M4,E4,P5);(A1,I2,M4,E4,P6);(A1,I2,M4,E4,P7);(A1,I2,M4,E4,P8);(A1,I2,M4,E4,P9);(A1,I2,M4,E4,P10);(A1,I2,M4,E4,P11);(A1,I2,M4,E4,P12);(A1,I2,M4,E4,P13);(A1,I2,M4,E4,P14);(A1,I2,M4,E4,P15);(A1,I2,M4,E4,P16);(A1,I2,M4,E4,P17);(A1,I2,M4,E4,P18);(A1,I2,M4,E4,P19);(A1,I2,M4,E4,P20);(A1,I2,M4,E5,P1);(A1,I2,M4,E5,P2);(A1,I2,M4,E5,P3);(A1,I2,M4,E5,P4);(A1,I2,M4,E5,P5);(A1,I2,M4,E5,P6);(A1,I2,M4,E5,P7);(A1,I2,M4,E5,P8);(A1,I2,M4,E5,P9);(A1,I2,M4,E5,P10);(A1,I2,M4,E5,P11);(A1,I2,M4,E5,P12);(A1,I2,M4,E5,P13);(A1,I2,M4,E5,P14);(A1,I2,M4,E5,P15);(A1,I2,M4,E5,P16);(A1,I2,M4,E5,P17);(A1,I2,M4,E5,P18);(A1,I2,M4,E5,P19);(A1,I2,M4,E5,P20);(A1,I2,M4,E6,P1);(A1,I2,M4,E6,P2);(A1,I2,M4,E6,P3);(A1,I2,M4,E6,P4);(A1,I2,M4,E6,P5);(A1,I2,M4,E6,P6);(A1,I2,M4,E6,P7);(A1,I2,M4,E6,P8);(A1,I2,M4,E6,P9);(A1,I2,M4,E6,P10);(A1,I2,M4,E6,P11);(A1,I2,M4,E6,P12);(A1,I2,M4,E6,P13);(A1,I2,M4,E6,P14);(A1,I2,M4,E6,P15);(A1,I2,M4,E6,P16);(A1,I2,M4,E6,P17);(A1,I2,M4,E6,P18);(A1,I2,M4,E6,P19);(A1,I2,M4,E6,P20);(A1,I2,M4,E7,P1);(A1,I2,M4,E7,P2);(A1,I2,M4,E7,P3);(A1,I2,M4,E7,P4);(A1,I2,M4,E7,P5);(A1,I2,M4,E7,P6);(A1,I2,M4,E7,P7);(A1,I2,M4,E7,P8);(A1,I2,M4,E7,P9);(A1,I2,M4,E7,P10);(A1,I2,M4,E7,P11);(A1,I2,M4,E7,P12);(A1,I2,M4,E7,P13);(A1,I2,M4,E7,P14);(A1,I2,M4,E7,P15);(A1,I2,M4,E7,P16);(A1,I2,M4,E7,P17);(A1,I2,M4,E7,P18);(A1,I2,M4,E7,P19);(A1,I2,M4,E7,P20);(A1,I2,M4,E8,P1);(A1,I2,M4,E8,P2);(A1,I2,M4,E8,P3);(A1,I2,M4,E8,P4);(A1,I2,M4,E8,P5);(A1,I2,M4,E8,P6);(A1,I2,M4,E8,P7);(A1,I2,M4,E8,P8);(A1,I2,M4,E8,P9);(A1,I2,M4,E8,P10);(A1,I2,M4,E8,P11);(A1,I2,M4,E8,P12);(A1,I2,M4,E8,P13);(A1,I2,M4,E8,P14);(A1,I2,M4,E8,P15);(A1,I2,M4,E8,P16);(A1,I2,M4,E8,P17);(A1,I2,M4,E8,P18);(A1,I2,M4,E8,P19);(A1,I2,M4,E8,P20);(A1,I2,M4,E9,P1);(A1,I2,M4,E9,P2);(A1,I2,M4,E9,P3);(A1,I2,M4,E9,P4);(A1,I2,M4,E9,P5);(A1,I2,M4,E9,P6);(A1,I2,M4,E9,P7);(A1,I2,M4,E9,P8);(A1,I2,M4,E9,P9);(A1,I2,M4,E9,P10);(A1,I2,M4,E9,P11);(A1,I2,M4,E9,P12);(A1,I2,M4,E9,P13);(A1,I2,M4,E9,P14);(A1,I2,M4,E9,P15);(A1,I2,M4,E9,P16);(A1,I2,M4,E9,P17);(A1,I2,M4,E9,P18);(A1,I2,M4,E9,P19);(A1,I2,M4,E9,P20);(A1,I2,M5,E1,P1);(A1,I2,M5,E1,P2);(A1,I2,M5,E1,P3);(A1,I2,M5,E1,P4);(A1,I2,M5,E1,P5);(A1,I2,M5,E1,P6);(A1,I2,M5,E1,P7);(A1,I2,M5,E1,P8);(A1,I2,M5,E1,P9);(A1,I2,M5,E1,P10);(A1,I2,M5,E1,P11);(A1,I2,M5,E1,P12);(A1,I2,M5,E1,P13);(A1,I2,M5,E1,P14);(A1,I2,M5,E1,P15);(A1,I2,M5,E1,P16);(A1,I2,M5,E1,P17);(A1,I2,M5,E1,P18);(A1,I2,M5,E1,P19);(A1,I2,M5,E1,P20);(A1,I2,M5,E2,P1);(A1,I2,M5,E2,P2);(A1,I2,M5,E2,P3);(A1,I2,M5,E2,P4);(A1,I2,M5,E2,P5);(A1,I2,M5,E2,P6);(A1,I2,M5,E2,P7);(A1,I2,M5,E2,P8);(A1,I2,M5,E2,P9);(A1,I2,M5,E2,P10);(A1,I2,M5,E2,P11);(A1,I2,M5,E2,P12);(A1,I2,M5,E2,P13);(A1,I2,M5,E2,P14);(A1,I2,M5,E2,P15);(A1,I2,M5,E2,P16);(A1,I2,M5,E2,P17);(A1,I2,M5,E2,P18);(A1,I2,M5,E2,P19);(A1,I2,M5,E2,P20);(A1,I2,M5,E3,P1);(A1,I2,M5,E3,P2);(A1,I2,M5,E3,P3);(A1,I2,M5,E3,P4);(A1,I2,M5,E3,P5);(A1,I2,M5,E3,P6);(A1,I2,M5,E3,P7);(A1,I2,M5,E3,P8);(A1,I2,M5,E3,P9);(A1,I2,M5,E3,P10);(A1,I2,M5,E3,P11);(A1,I2,M5,E3,P12);(A1,I2,M5,E3,P13);(A1,I2,M5,E3,P14);(A1,I2,M5,E3,P15);(A1,I2,M5,E3,P16);(A1,I2,M5,E3,P17);(A1,I2,M5,E3,P18);(A1,I2,M5,E3,P19);(A1,I2,M5,E3,P20);(A1,I2,M5,E4,P1);(A1,I2,M5,E4,P2);(A1,I2,M5,E4,P3);(A1,I2,M5,E4,P4);(A1,I2,M5,E4,P5);(A1,I2,M5,E4,P6);(A1,I2,M5,E4,P7);(A1,I2,M5,E4,P8);(A1,I2,M5,E4,P9);(A1,I2,M5,E4,P10);(A1,I2,M5,E4,P11);(A1,I2,M5,E4,P12);(A1,I2,M5,E4,P13);(A1,I2,M5,E4,P14);(A1,I2,M5,E4,P15);(A1,I2,M5,E4,P16);(A1,I2,M5,E4,P17);(A1,I2,M5,E4,P18);(A1,I2,M5,E4,P19);(A1,I2,M5,E4,P20);(A1,I2,M5,E5,P1);(A1,I2,M5,E5,P2);(A1,I2,M5,E5,P3);(A1,I2,M5,E5,P4);(A1,I2,M5,E5,P5);(A1,I2,M5,E5,P6);(A1,I2,M5,E5,P7);(A1,I2,M5,E5,P8);(A1,I2,M5,E5,P9);(A1,I2,M5,E5,P10);(A1,I2,M5,E5,P11);(A1,I2,M5,E5,P12);(A1,I2,M5,E5,P13);(A1,I2,M5,E5,P14);(A1,I2,M5,E5,P15);(A1,I2,M5,E5,P16);(A1,I2,M5,E5,P17);(A1,I2,M5,E5,P18);(A1,I2,M5,E5,P19);(A1,I2,M5,E5,P20);(A1,I2,M5,E6,P1);(A1,I2,M5,E6,P2);(A1,I2,M5,E6,P3);(A1,I2,M5,E6,P4);(A1,I2,M5,E6,P5);(A1,I2,M5,E6,P6);(A1,I2,M5,E6,P7);(A1,I2,M5,E6,P8);(A1,I2,M5,E6,P9);(A1,I2,M5,E6,P10);(A1,I2,M5,E6,P11);(A1,I2,M5,E6,P12);(A1,I2,M5,E6,P13);(A1,I2,M5,E6,P14);(A1,I2,M5,E6,P15);(A1,I2,M5,E6,P16);(A1,I2,M5,E6,P17);(A1,I2,M5,E6,P18);(A1,I2,M5,E6,P19);(A1,I2,M5,E6,P20);(A1,I2,M5,E7,P1);(A1,I2,M5,E7,P2);(A1,I2,M5,E7,P3);(A1,I2,M5,E7,P4);(A1,I2,M5,E7,P5);(A1,I2,M5,E7,P6);(A1,I2,M5,E7,P7);(A1,I2,M5,E7,P8);(A1,I2,M5,E7,P9);(A1,I2,M5,E7,P10);(A1,I2,M5,E7,P11);(A1,I2,M5,E7,P12);(A1,I2,M5,E7,P13);(A1,I2,M5,E7,P14);(A1,I2,M5,E7,P15);(A1,I2,M5,E7,P16);(A1,I2,M5,E7,P17);(A1,I2,M5,E7,P18);(A1,I2,M5,E7,P19);(A1,I2,M5,E7,P20);(A1,I2,M5,E8,P1);(A1,I2,M5,E8,P2);(A1,I2,M5,E8,P3);(A1,I2,M5,E8,P4);(A1,I2,M5,E8,P5);(A1,I2,M5,E8,P6);(A1,I2,M5,E8,P7);(A1,I2,M5,E8,P8);(A1,I2,M5,E8,P9);(A1,I2,M5,E8,P10);(A1,I2,M5,E8,P11);(A1,I2,M5,E8,P12);(A1,I2,M5,E8,P13);(A1,I2,M5,E8,P14);(A1,I2,M5,E8,P15);(A1,I2,M5,E8,P16);(A1,I2,M5,E8,P17);(A1,I2,M5,E8,P18);(A1,I2,M5,E8,P19);(A1,I2,M5,E8,P20);(A1,I2,M5,E9,P1);(A1,I2,M5,E9,P2);(A1,I2,M5,E9,P3);(A1,I2,M5,E9,P4);(A1,I2,M5,E9,P5);(A1,I2,M5,E9,P6);(A1,I2,M5,E9,P7);(A1,I2,M5,E9,P8);(A1,I2,M5,E9,P9);(A1,I2,M5,E9,P10);(A1,I2,M5,E9,P11);(A1,I2,M5,E9,P12);(A1,I2,M5,E9,P13);(A1,I2,M5,E9,P14);(A1,I2,M5,E9,P15);(A1,I2,M5,E9,P16);(A1,I2,M5,E9,P17);(A1,I2,M5,E9,P18);(A1,I2,M5,E9,P19);(A1,I2,M5,E9,P20);(A1,I2,M6,E1,P1);(A1,I2,M6,E1,P2);(A1,I2,M6,E1,P3);(A1,I2,M6,E1,P4);(A1,I2,M6,E1,P5);(A1,I2,M6,E1,P6);(A1,I2,M6,E1,P7);(A1,I2,M6,E1,P8);(A1,I2,M6,E1,P9);(A1,I2,M6,E1,P10);(A1,I2,M6,E1,P11);(A1,I2,M6,E1,P12);(A1,I2,M6,E1,P13);(A1,I2,M6,E1,P14);(A1,I2,M6,E1,P15);(A1,I2,M6,E1,P16);(A1,I2,M6,E1,P17);(A1,I2,M6,E1,P18);(A1,I2,M6,E1,P19);(A1,I2,M6,E1,P20);(A1,I2,M6,E2,P1);(A1,I2,M6,E2,P2);(A1,I2,M6,E2,P3);(A1,I2,M6,E2,P4);(A1,I2,M6,E2,P5);(A1,I2,M6,E2,P6);(A1,I2,M6,E2,P7);(A1,I2,M6,E2,P8);(A1,I2,M6,E2,P9);(A1,I2,M6,E2,P10);(A1,I2,M6,E2,P11);(A1,I2,M6,E2,P12);(A1,I2,M6,E2,P13);(A1,I2,M6,E2,P14);(A1,I2,M6,E2,P15);(A1,I2,M6,E2,P16);(A1,I2,M6,E2,P17);(A1,I2,M6,E2,P18);(A1,I2,M6,E2,P19);(A1,I2,M6,E2,P20);(A1,I2,M6,E3,P1);(A1,I2,M6,E3,P2);(A1,I2,M6,E3,P3);(A1,I2,M6,E3,P4);(A1,I2,M6,E3,P5);(A1,I2,M6,E3,P6);(A1,I2,M6,E3,P7);(A1,I2,M6,E3,P8);(A1,I2,M6,E3,P9);(A1,I2,M6,E3,P10);(A1,I2,M6,E3,P11);(A1,I2,M6,E3,P12);(A1,I2,M6,E3,P13);(A1,I2,M6,E3,P14);(A1,I2,M6,E3,P15);(A1,I2,M6,E3,P16);(A1,I2,M6,E3,P17);(A1,I2,M6,E3,P18);(A1,I2,M6,E3,P19);(A1,I2,M6,E3,P20);(A1,I2,M6,E4,P1);(A1,I2,M6,E4,P2);(A1,I2,M6,E4,P3);(A1,I2,M6,E4,P4);(A1,I2,M6,E4,P5);(A1,I2,M6,E4,P6);(A1,I2,M6,E4,P7);(A1,I2,M6,E4,P8);(A1,I2,M6,E4,P9);(A1,I2,M6,E4,P10);(A1,I2,M6,E4,P11);(A1,I2,M6,E4,P12);(A1,I2,M6,E4,P13);(A1,I2,M6,E4,P14);(A1,I2,M6,E4,P15);(A1,I2,M6,E4,P16);(A1,I2,M6,E4,P17);(A1,I2,M6,E4,P18);(A1,I2,M6,E4,P19);(A1,I2,M6,E4,P20);(A1,I2,M6,E5,P1);(A1,I2,M6,E5,P2);(A1,I2,M6,E5,P3);(A1,I2,M6,E5,P4);(A1,I2,M6,E5,P5);(A1,I2,M6,E5,P6);(A1,I2,M6,E5,P7);(A1,I2,M6,E5,P8);(A1,I2,M6,E5,P9);(A1,I2,M6,E5,P10);(A1,I2,M6,E5,P11);(A1,I2,M6,E5,P12);(A1,I2,M6,E5,P13);(A1,I2,M6,E5,P14);(A1,I2,M6,E5,P15);(A1,I2,M6,E5,P16);(A1,I2,M6,E5,P17);(A1,I2,M6,E5,P18);(A1,I2,M6,E5,P19);(A1,I2,M6,E5,P20);(A1,I2,M6,E6,P1);(A1,I2,M6,E6,P2);(A1,I2,M6,E6,P3);(A1,I2,M6,E6,P4);(A1,I2,M6,E6,P5);(A1,I2,M6,E6,P6);(A1,I2,M6,E6,P7);(A1,I2,M6,E6,P8);(A1,I2,M6,E6,P9);(A1,I2,M6,E6,P10);(A1,I2,M6,E6,P11);(A1,I2,M6,E6,P12);(A1,I2,M6,E6,P13);(A1,I2,M6,E6,P14);(A1,I2,M6,E6,P15);(A1,I2,M6,E6,P16);(A1,I2,M6,E6,P17);(A1,I2,M6,E6,P18);(A1,I2,M6,E6,P19);(A1,I2,M6,E6,P20);(A1,I2,M6,E7,P1);(A1,I2,M6,E7,P2);(A1,I2,M6,E7,P3);(A1,I2,M6,E7,P4);(A1,I2,M6,E7,P5);(A1,I2,M6,E7,P6);(A1,I2,M6,E7,P7);(A1,I2,M6,E7,P8);(A1,I2,M6,E7,P9);(A1,I2,M6,E7,P10);(A1,I2,M6,E7,P11);(A1,I2,M6,E7,P12);(A1,I2,M6,E7,P13);(A1,I2,M6,E7,P14);(A1,I2,M6,E7,P15);(A1,I2,M6,E7,P16);(A1,I2,M6,E7,P17);(A1,I2,M6,E7,P18);(A1,I2,M6,E7,P19);(A1,I2,M6,E7,P20);(A1,I2,M6,E8,P1);(A1,I2,M6,E8,P2);(A1,I2,M6,E8,P3);(A1,I2,M6,E8,P4);(A1,I2,M6,E8,P5);(A1,I2,M6,E8,P6);(A1,I2,M6,E8,P7);(A1,I2,M6,E8,P8);(A1,I2,M6,E8,P9);(A1,I2,M6,E8,P10);(A1,I2,M6,E8,P11);(A1,I2,M6,E8,P12);(A1,I2,M6,E8,P13);(A1,I2,M6,E8,P14);(A1,I2,M6,E8,P15);(A1,I2,M6,E8,P16);(A1,I2,M6,E8,P17);(A1,I2,M6,E8,P18);(A1,I2,M6,E8,P19);(A1,I2,M6,E8,P20);(A1,I2,M6,E9,P1);(A1,I2,M6,E9,P2);(A1,I2,M6,E9,P3);(A1,I2,M6,E9,P4);(A1,I2,M6,E9,P5);(A1,I2,M6,E9,P6);(A1,I2,M6,E9,P7);(A1,I2,M6,E9,P8);(A1,I2,M6,E9,P9);(A1,I2,M6,E9,P10);(A1,I2,M6,E9,P11);(A1,I2,M6,E9,P12);(A1,I2,M6,E9,P13);(A1,I2,M6,E9,P14);(A1,I2,M6,E9,P15);(A1,I2,M6,E9,P16);(A1,I2,M6,E9,P17);(A1,I2,M6,E9,P18);(A1,I2,M6,E9,P19);(A1,I2,M6,E9,P20);(A1,I3,M1,E1,P1);(A1,I3,M1,E1,P2);(A1,I3,M1,E1,P3);(A1,I3,M1,E1,P4);(A1,I3,M1,E1,P5);(A1,I3,M1,E1,P6);(A1,I3,M1,E1,P7);(A1,I3,M1,E1,P8);(A1,I3,M1,E1,P9);(A1,I3,M1,E1,P10);(A1,I3,M1,E1,P11);(A1,I3,M1,E1,P12);(A1,I3,M1,E1,P13);(A1,I3,M1,E1,P14);(A1,I3,M1,E1,P15);(A1,I3,M1,E1,P16);(A1,I3,M1,E1,P17);(A1,I3,M1,E1,P18);(A1,I3,M1,E1,P19);(A1,I3,M1,E1,P20);(A1,I3,M1,E2,P1);(A1,I3,M1,E2,P2);(A1,I3,M1,E2,P3);(A1,I3,M1,E2,P4);(A1,I3,M1,E2,P5);(A1,I3,M1,E2,P6);(A1,I3,M1,E2,P7);(A1,I3,M1,E2,P8);(A1,I3,M1,E2,P9);(A1,I3,M1,E2,P10);(A1,I3,M1,E2,P11);(A1,I3,M1,E2,P12);(A1,I3,M1,E2,P13);(A1,I3,M1,E2,P14);(A1,I3,M1,E2,P15);(A1,I3,M1,E2,P16);(A1,I3,M1,E2,P17);(A1,I3,M1,E2,P18);(A1,I3,M1,E2,P19);(A1,I3,M1,E2,P20);(A1,I3,M1,E3,P1);(A1,I3,M1,E3,P2);(A1,I3,M1,E3,P3);(A1,I3,M1,E3,P4);(A1,I3,M1,E3,P5);(A1,I3,M1,E3,P6);(A1,I3,M1,E3,P7);(A1,I3,M1,E3,P8);(A1,I3,M1,E3,P9);(A1,I3,M1,E3,P10);(A1,I3,M1,E3,P11);(A1,I3,M1,E3,P12);(A1,I3,M1,E3,P13);(A1,I3,M1,E3,P14);(A1,I3,M1,E3,P15);(A1,I3,M1,E3,P16);(A1,I3,M1,E3,P17);(A1,I3,M1,E3,P18);(A1,I3,M1,E3,P19);(A1,I3,M1,E3,P20);(A1,I3,M1,E4,P1);(A1,I3,M1,E4,P2);(A1,I3,M1,E4,P3);(A1,I3,M1,E4,P4);(A1,I3,M1,E4,P5);(A1,I3,M1,E4,P6);(A1,I3,M1,E4,P7);(A1,I3,M1,E4,P8);(A1,I3,M1,E4,P9);(A1,I3,M1,E4,P10);(A1,I3,M1,E4,P11);(A1,I3,M1,E4,P12);(A1,I3,M1,E4,P13);(A1,I3,M1,E4,P14);(A1,I3,M1,E4,P15);(A1,I3,M1,E4,P16);(A1,I3,M1,E4,P17);(A1,I3,M1,E4,P18);(A1,I3,M1,E4,P19);(A1,I3,M1,E4,P20);(A1,I3,M1,E5,P1);(A1,I3,M1,E5,P2);(A1,I3,M1,E5,P3);(A1,I3,M1,E5,P4);(A1,I3,M1,E5,P5);(A1,I3,M1,E5,P6);(A1,I3,M1,E5,P7);(A1,I3,M1,E5,P8);(A1,I3,M1,E5,P9);(A1,I3,M1,E5,P10);(A1,I3,M1,E5,P11);(A1,I3,M1,E5,P12);(A1,I3,M1,E5,P13);(A1,I3,M1,E5,P14);(A1,I3,M1,E5,P15);(A1,I3,M1,E5,P16);(A1,I3,M1,E5,P17);(A1,I3,M1,E5,P18);(A1,I3,M1,E5,P19);(A1,I3,M1,E5,P20);(A1,I3,M1,E6,P1);(A1,I3,M1,E6,P2);(A1,I3,M1,E6,P3);(A1,I3,M1,E6,P4);(A1,I3,M1,E6,P5);(A1,I3,M1,E6,P6);(A1,I3,M1,E6,P7);(A1,I3,M1,E6,P8);(A1,I3,M1,E6,P9);(A1,I3,M1,E6,P10);(A1,I3,M1,E6,P11);(A1,I3,M1,E6,P12);(A1,I3,M1,E6,P13);(A1,I3,M1,E6,P14);(A1,I3,M1,E6,P15);(A1,I3,M1,E6,P16);(A1,I3,M1,E6,P17);(A1,I3,M1,E6,P18);(A1,I3,M1,E6,P19);(A1,I3,M1,E6,P20);(A1,I3,M1,E7,P1);(A1,I3,M1,E7,P2);(A1,I3,M1,E7,P3);(A1,I3,M1,E7,P4);(A1,I3,M1,E7,P5);(A1,I3,M1,E7,P6);(A1,I3,M1,E7,P7);(A1,I3,M1,E7,P8);(A1,I3,M1,E7,P9);(A1,I3,M1,E7,P10);(A1,I3,M1,E7,P11);(A1,I3,M1,E7,P12);(A1,I3,M1,E7,P13);(A1,I3,M1,E7,P14);(A1,I3,M1,E7,P15);(A1,I3,M1,E7,P16);(A1,I3,M1,E7,P17);(A1,I3,M1,E7,P18);(A1,I3,M1,E7,P19);(A1,I3,M1,E7,P20);(A1,I3,M1,E8,P1);(A1,I3,M1,E8,P2);(A1,I3,M1,E8,P3);(A1,I3,M1,E8,P4);(A1,I3,M1,E8,P5);(A1,I3,M1,E8,P6);(A1,I3,M1,E8,P7);(A1,I3,M1,E8,P8);(A1,I3,M1,E8,P9);(A1,I3,M1,E8,P10);(A1,I3,M1,E8,P11);(A1,I3,M1,E8,P12);(A1,I3,M1,E8,P13);(A1,I3,M1,E8,P14);(A1,I3,M1,E8,P15);(A1,I3,M1,E8,P16);(A1,I3,M1,E8,P17);(A1,I3,M1,E8,P18);(A1,I3,M1,E8,P19);(A1,I3,M1,E8,P20);(A1,I3,M1,E9,P1);(A1,I3,M1,E9,P2);(A1,I3,M1,E9,P3);(A1,I3,M1,E9,P4);(A1,I3,M1,E9,P5);(A1,I3,M1,E9,P6);(A1,I3,M1,E9,P7);(A1,I3,M1,E9,P8);(A1,I3,M1,E9,P9);(A1,I3,M1,E9,P10);(A1,I3,M1,E9,P11);(A1,I3,M1,E9,P12);(A1,I3,M1,E9,P13);(A1,I3,M1,E9,P14);(A1,I3,M1,E9,P15);(A1,I3,M1,E9,P16);(A1,I3,M1,E9,P17);(A1,I3,M1,E9,P18);(A1,I3,M1,E9,P19);(A1,I3,M1,E9,P20);(A1,I3,M2,E1,P1);(A1,I3,M2,E1,P2);(A1,I3,M2,E1,P3);(A1,I3,M2,E1,P4);(A1,I3,M2,E1,P5);(A1,I3,M2,E1,P6);(A1,I3,M2,E1,P7);(A1,I3,M2,E1,P8);(A1,I3,M2,E1,P9);(A1,I3,M2,E1,P10);(A1,I3,M2,E1,P11);(A1,I3,M2,E1,P12);(A1,I3,M2,E1,P13);(A1,I3,M2,E1,P14);(A1,I3,M2,E1,P15);(A1,I3,M2,E1,P16);(A1,I3,M2,E1,P17);(A1,I3,M2,E1,P18);(A1,I3,M2,E1,P19);(A1,I3,M2,E1,P20);(A1,I3,M2,E2,P1);(A1,I3,M2,E2,P2);(A1,I3,M2,E2,P3);(A1,I3,M2,E2,P4);(A1,I3,M2,E2,P5);(A1,I3,M2,E2,P6);(A1,I3,M2,E2,P7);(A1,I3,M2,E2,P8);(A1,I3,M2,E2,P9);(A1,I3,M2,E2,P10);(A1,I3,M2,E2,P11);(A1,I3,M2,E2,P12);(A1,I3,M2,E2,P13);(A1,I3,M2,E2,P14);(A1,I3,M2,E2,P15);(A1,I3,M2,E2,P16);(A1,I3,M2,E2,P17);(A1,I3,M2,E2,P18);(A1,I3,M2,E2,P19);(A1,I3,M2,E2,P20);(A1,I3,M2,E3,P1);(A1,I3,M2,E3,P2);(A1,I3,M2,E3,P3);(A1,I3,M2,E3,P4);(A1,I3,M2,E3,P5);(A1,I3,M2,E3,P6);(A1,I3,M2,E3,P7);(A1,I3,M2,E3,P8);(A1,I3,M2,E3,P9);(A1,I3,M2,E3,P10);(A1,I3,M2,E3,P11);(A1,I3,M2,E3,P12);(A1,I3,M2,E3,P13);(A1,I3,M2,E3,P14);(A1,I3,M2,E3,P15);(A1,I3,M2,E3,P16);(A1,I3,M2,E3,P17);(A1,I3,M2,E3,P18);(A1,I3,M2,E3,P19);(A1,I3,M2,E3,P20);(A1,I3,M2,E4,P1);(A1,I3,M2,E4,P2);(A1,I3,M2,E4,P3);(A1,I3,M2,E4,P4);(A1,I3,M2,E4,P5);(A1,I3,M2,E4,P6);(A1,I3,M2,E4,P7);(A1,I3,M2,E4,P8);(A1,I3,M2,E4,P9);(A1,I3,M2,E4,P10);(A1,I3,M2,E4,P11);(A1,I3,M2,E4,P12);(A1,I3,M2,E4,P13);(A1,I3,M2,E4,P14);(A1,I3,M2,E4,P15);(A1,I3,M2,E4,P16);(A1,I3,M2,E4,P17);(A1,I3,M2,E4,P18);(A1,I3,M2,E4,P19);(A1,I3,M2,E4,P20);(A1,I3,M2,E5,P1);(A1,I3,M2,E5,P2);(A1,I3,M2,E5,P3);(A1,I3,M2,E5,P4);(A1,I3,M2,E5,P5);(A1,I3,M2,E5,P6);(A1,I3,M2,E5,P7);(A1,I3,M2,E5,P8);(A1,I3,M2,E5,P9);(A1,I3,M2,E5,P10);(A1,I3,M2,E5,P11);(A1,I3,M2,E5,P12);(A1,I3,M2,E5,P13);(A1,I3,M2,E5,P14);(A1,I3,M2,E5,P15);(A1,I3,M2,E5,P16);(A1,I3,M2,E5,P17);(A1,I3,M2,E5,P18);(A1,I3,M2,E5,P19);(A1,I3,M2,E5,P20);(A1,I3,M2,E6,P1);(A1,I3,M2,E6,P2);(A1,I3,M2,E6,P3);(A1,I3,M2,E6,P4);(A1,I3,M2,E6,P5);(A1,I3,M2,E6,P6);(A1,I3,M2,E6,P7);(A1,I3,M2,E6,P8);(A1,I3,M2,E6,P9);(A1,I3,M2,E6,P10);(A1,I3,M2,E6,P11);(A1,I3,M2,E6,P12);(A1,I3,M2,E6,P13);(A1,I3,M2,E6,P14);(A1,I3,M2,E6,P15);(A1,I3,M2,E6,P16);(A1,I3,M2,E6,P17);(A1,I3,M2,E6,P18);(A1,I3,M2,E6,P19);(A1,I3,M2,E6,P20);(A1,I3,M2,E7,P1);(A1,I3,M2,E7,P2);(A1,I3,M2,E7,P3);(A1,I3,M2,E7,P4);(A1,I3,M2,E7,P5);(A1,I3,M2,E7,P6);(A1,I3,M2,E7,P7);(A1,I3,M2,E7,P8);(A1,I3,M2,E7,P9);(A1,I3,M2,E7,P10);(A1,I3,M2,E7,P11);(A1,I3,M2,E7,P12);(A1,I3,M2,E7,P13);(A1,I3,M2,E7,P14);(A1,I3,M2,E7,P15);(A1,I3,M2,E7,P16);(A1,I3,M2,E7,P17);(A1,I3,M2,E7,P18);(A1,I3,M2,E7,P19);(A1,I3,M2,E7,P20);(A1,I3,M2,E8,P1);(A1,I3,M2,E8,P2);(A1,I3,M2,E8,P3);(A1,I3,M2,E8,P4);(A1,I3,M2,E8,P5);(A1,I3,M2,E8,P6);(A1,I3,M2,E8,P7);(A1,I3,M2,E8,P8);(A1,I3,M2,E8,P9);(A1,I3,M2,E8,P10);(A1,I3,M2,E8,P11);(A1,I3,M2,E8,P12);(A1,I3,M2,E8,P13);(A1,I3,M2,E8,P14);(A1,I3,M2,E8,P15);(A1,I3,M2,E8,P16);(A1,I3,M2,E8,P17);(A1,I3,M2,E8,P18);(A1,I3,M2,E8,P19);(A1,I3,M2,E8,P20);(A1,I3,M2,E9,P1);(A1,I3,M2,E9,P2);(A1,I3,M2,E9,P3);(A1,I3,M2,E9,P4);(A1,I3,M2,E9,P5);(A1,I3,M2,E9,P6);(A1,I3,M2,E9,P7);(A1,I3,M2,E9,P8);(A1,I3,M2,E9,P9);(A1,I3,M2,E9,P10);(A1,I3,M2,E9,P11);(A1,I3,M2,E9,P12);(A1,I3,M2,E9,P13);(A1,I3,M2,E9,P14);(A1,I3,M2,E9,P15);(A1,I3,M2,E9,P16);(A1,I3,M2,E9,P17);(A1,I3,M2,E9,P18);(A1,I3,M2,E9,P19);(A1,I3,M2,E9,P20);(A1,I3,M3,E1,P1);(A1,I3,M3,E1,P2);(A1,I3,M3,E1,P3);(A1,I3,M3,E1,P4);(A1,I3,M3,E1,P5);(A1,I3,M3,E1,P6);(A1,I3,M3,E1,P7);(A1,I3,M3,E1,P8);(A1,I3,M3,E1,P9);(A1,I3,M3,E1,P10);(A1,I3,M3,E1,P11);(A1,I3,M3,E1,P12);(A1,I3,M3,E1,P13);(A1,I3,M3,E1,P14);(A1,I3,M3,E1,P15);(A1,I3,M3,E1,P16);(A1,I3,M3,E1,P17);(A1,I3,M3,E1,P18);(A1,I3,M3,E1,P19);(A1,I3,M3,E1,P20);(A1,I3,M3,E2,P1);(A1,I3,M3,E2,P2);(A1,I3,M3,E2,P3);(A1,I3,M3,E2,P4);(A1,I3,M3,E2,P5);(A1,I3,M3,E2,P6);(A1,I3,M3,E2,P7);(A1,I3,M3,E2,P8);(A1,I3,M3,E2,P9);(A1,I3,M3,E2,P10);(A1,I3,M3,E2,P11);(A1,I3,M3,E2,P12);(A1,I3,M3,E2,P13);(A1,I3,M3,E2,P14);(A1,I3,M3,E2,P15);(A1,I3,M3,E2,P16);(A1,I3,M3,E2,P17);(A1,I3,M3,E2,P18);(A1,I3,M3,E2,P19);(A1,I3,M3,E2,P20);(A1,I3,M3,E3,P1);(A1,I3,M3,E3,P2);(A1,I3,M3,E3,P3);(A1,I3,M3,E3,P4);(A1,I3,M3,E3,P5);(A1,I3,M3,E3,P6);(A1,I3,M3,E3,P7);(A1,I3,M3,E3,P8);(A1,I3,M3,E3,P9);(A1,I3,M3,E3,P10);(A1,I3,M3,E3,P11);(A1,I3,M3,E3,P12);(A1,I3,M3,E3,P13);(A1,I3,M3,E3,P14);(A1,I3,M3,E3,P15);(A1,I3,M3,E3,P16);(A1,I3,M3,E3,P17);(A1,I3,M3,E3,P18);(A1,I3,M3,E3,P19);(A1,I3,M3,E3,P20);(A1,I3,M3,E4,P1);(A1,I3,M3,E4,P2);(A1,I3,M3,E4,P3);(A1,I3,M3,E4,P4);(A1,I3,M3,E4,P5);(A1,I3,M3,E4,P6);(A1,I3,M3,E4,P7);(A1,I3,M3,E4,P8);(A1,I3,M3,E4,P9);(A1,I3,M3,E4,P10);(A1,I3,M3,E4,P11);(A1,I3,M3,E4,P12);(A1,I3,M3,E4,P13);(A1,I3,M3,E4,P14);(A1,I3,M3,E4,P15);(A1,I3,M3,E4,P16);(A1,I3,M3,E4,P17);(A1,I3,M3,E4,P18);(A1,I3,M3,E4,P19);(A1,I3,M3,E4,P20);(A1,I3,M3,E5,P1);(A1,I3,M3,E5,P2);(A1,I3,M3,E5,P3);(A1,I3,M3,E5,P4);(A1,I3,M3,E5,P5);(A1,I3,M3,E5,P6);(A1,I3,M3,E5,P7);(A1,I3,M3,E5,P8);(A1,I3,M3,E5,P9);(A1,I3,M3,E5,P10);(A1,I3,M3,E5,P11);(A1,I3,M3,E5,P12);(A1,I3,M3,E5,P13);(A1,I3,M3,E5,P14);(A1,I3,M3,E5,P15);(A1,I3,M3,E5,P16);(A1,I3,M3,E5,P17);(A1,I3,M3,E5,P18);(A1,I3,M3,E5,P19);(A1,I3,M3,E5,P20);(A1,I3,M3,E6,P1);(A1,I3,M3,E6,P2);(A1,I3,M3,E6,P3);(A1,I3,M3,E6,P4);(A1,I3,M3,E6,P5);(A1,I3,M3,E6,P6);(A1,I3,M3,E6,P7);(A1,I3,M3,E6,P8);(A1,I3,M3,E6,P9);(A1,I3,M3,E6,P10);(A1,I3,M3,E6,P11);(A1,I3,M3,E6,P12);(A1,I3,M3,E6,P13);(A1,I3,M3,E6,P14);(A1,I3,M3,E6,P15);(A1,I3,M3,E6,P16);(A1,I3,M3,E6,P17);(A1,I3,M3,E6,P18);(A1,I3,M3,E6,P19);(A1,I3,M3,E6,P20);(A1,I3,M3,E7,P1);(A1,I3,M3,E7,P2);(A1,I3,M3,E7,P3);(A1,I3,M3,E7,P4);(A1,I3,M3,E7,P5);(A1,I3,M3,E7,P6);(A1,I3,M3,E7,P7);(A1,I3,M3,E7,P8);(A1,I3,M3,E7,P9);(A1,I3,M3,E7,P10);(A1,I3,M3,E7,P11);(A1,I3,M3,E7,P12);(A1,I3,M3,E7,P13);(A1,I3,M3,E7,P14);(A1,I3,M3,E7,P15);(A1,I3,M3,E7,P16);(A1,I3,M3,E7,P17);(A1,I3,M3,E7,P18);(A1,I3,M3,E7,P19);(A1,I3,M3,E7,P20);(A1,I3,M3,E8,P1);(A1,I3,M3,E8,P2);(A1,I3,M3,E8,P3);(A1,I3,M3,E8,P4);(A1,I3,M3,E8,P5);(A1,I3,M3,E8,P6);(A1,I3,M3,E8,P7);(A1,I3,M3,E8,P8);(A1,I3,M3,E8,P9);(A1,I3,M3,E8,P10);(A1,I3,M3,E8,P11);(A1,I3,M3,E8,P12);(A1,I3,M3,E8,P13);(A1,I3,M3,E8,P14);(A1,I3,M3,E8,P15);(A1,I3,M3,E8,P16);(A1,I3,M3,E8,P17);(A1,I3,M3,E8,P18);(A1,I3,M3,E8,P19);(A1,I3,M3,E8,P20);(A1,I3,M3,E9,P1);(A1,I3,M3,E9,P2);(A1,I3,M3,E9,P3);(A1,I3,M3,E9,P4);(A1,I3,M3,E9,P5);(A1,I3,M3,E9,P6);(A1,I3,M3,E9,P7);(A1,I3,M3,E9,P8);(A1,I3,M3,E9,P9);(A1,I3,M3,E9,P10);(A1,I3,M3,E9,P11);(A1,I3,M3,E9,P12);(A1,I3,M3,E9,P13);(A1,I3,M3,E9,P14);(A1,I3,M3,E9,P15);(A1,I3,M3,E9,P16);(A1,I3,M3,E9,P17);(A1,I3,M3,E9,P18);(A1,I3,M3,E9,P19);(A1,I3,M3,E9,P20);(A1,I3,M4,E1,P1);(A1,I3,M4,E1,P2);(A1,I3,M4,E1,P3);(A1,I3,M4,E1,P4);(A1,I3,M4,E1,P5);(A1,I3,M4,E1,P6);(A1,I3,M4,E1,P7);(A1,I3,M4,E1,P8);(A1,I3,M4,E1,P9);(A1,I3,M4,E1,P10);(A1,I3,M4,E1,P11);(A1,I3,M4,E1,P12);(A1,I3,M4,E1,P13);(A1,I3,M4,E1,P14);(A1,I3,M4,E1,P15);(A1,I3,M4,E1,P16);(A1,I3,M4,E1,P17);(A1,I3,M4,E1,P18);(A1,I3,M4,E1,P19);(A1,I3,M4,E1,P20);(A1,I3,M4,E2,P1);(A1,I3,M4,E2,P2);(A1,I3,M4,E2,P3);(A1,I3,M4,E2,P4);(A1,I3,M4,E2,P5);(A1,I3,M4,E2,P6);(A1,I3,M4,E2,P7);(A1,I3,M4,E2,P8);(A1,I3,M4,E2,P9);(A1,I3,M4,E2,P10);(A1,I3,M4,E2,P11);(A1,I3,M4,E2,P12);(A1,I3,M4,E2,P13);(A1,I3,M4,E2,P14);(A1,I3,M4,E2,P15);(A1,I3,M4,E2,P16);(A1,I3,M4,E2,P17);(A1,I3,M4,E2,P18);(A1,I3,M4,E2,P19);(A1,I3,M4,E2,P20);(A1,I3,M4,E3,P1);(A1,I3,M4,E3,P2);(A1,I3,M4,E3,P3);(A1,I3,M4,E3,P4);(A1,I3,M4,E3,P5);(A1,I3,M4,E3,P6);(A1,I3,M4,E3,P7);(A1,I3,M4,E3,P8);(A1,I3,M4,E3,P9);(A1,I3,M4,E3,P10);(A1,I3,M4,E3,P11);(A1,I3,M4,E3,P12);(A1,I3,M4,E3,P13);(A1,I3,M4,E3,P14);(A1,I3,M4,E3,P15);(A1,I3,M4,E3,P16);(A1,I3,M4,E3,P17);(A1,I3,M4,E3,P18);(A1,I3,M4,E3,P19);(A1,I3,M4,E3,P20);(A1,I3,M4,E4,P1);(A1,I3,M4,E4,P2);(A1,I3,M4,E4,P3);(A1,I3,M4,E4,P4);(A1,I3,M4,E4,P5);(A1,I3,M4,E4,P6);(A1,I3,M4,E4,P7);(A1,I3,M4,E4,P8);(A1,I3,M4,E4,P9);(A1,I3,M4,E4,P10);(A1,I3,M4,E4,P11);(A1,I3,M4,E4,P12);(A1,I3,M4,E4,P13);(A1,I3,M4,E4,P14);(A1,I3,M4,E4,P15);(A1,I3,M4,E4,P16);(A1,I3,M4,E4,P17);(A1,I3,M4,E4,P18);(A1,I3,M4,E4,P19);(A1,I3,M4,E4,P20);(A1,I3,M4,E5,P1);(A1,I3,M4,E5,P2);(A1,I3,M4,E5,P3);(A1,I3,M4,E5,P4);(A1,I3,M4,E5,P5);(A1,I3,M4,E5,P6);(A1,I3,M4,E5,P7);(A1,I3,M4,E5,P8);(A1,I3,M4,E5,P9);(A1,I3,M4,E5,P10);(A1,I3,M4,E5,P11);(A1,I3,M4,E5,P12);(A1,I3,M4,E5,P13);(A1,I3,M4,E5,P14);(A1,I3,M4,E5,P15);(A1,I3,M4,E5,P16);(A1,I3,M4,E5,P17);(A1,I3,M4,E5,P18);(A1,I3,M4,E5,P19);(A1,I3,M4,E5,P20);(A1,I3,M4,E6,P1);(A1,I3,M4,E6,P2);(A1,I3,M4,E6,P3);(A1,I3,M4,E6,P4);(A1,I3,M4,E6,P5);(A1,I3,M4,E6,P6);(A1,I3,M4,E6,P7);(A1,I3,M4,E6,P8);(A1,I3,M4,E6,P9);(A1,I3,M4,E6,P10);(A1,I3,M4,E6,P11);(A1,I3,M4,E6,P12);(A1,I3,M4,E6,P13);(A1,I3,M4,E6,P14);(A1,I3,M4,E6,P15);(A1,I3,M4,E6,P16);(A1,I3,M4,E6,P17);(A1,I3,M4,E6,P18);(A1,I3,M4,E6,P19);(A1,I3,M4,E6,P20);(A1,I3,M4,E7,P1);(A1,I3,M4,E7,P2);(A1,I3,M4,E7,P3);(A1,I3,M4,E7,P4);(A1,I3,M4,E7,P5);(A1,I3,M4,E7,P6);(A1,I3,M4,E7,P7);(A1,I3,M4,E7,P8);(A1,I3,M4,E7,P9);(A1,I3,M4,E7,P10);(A1,I3,M4,E7,P11);(A1,I3,M4,E7,P12);(A1,I3,M4,E7,P13);(A1,I3,M4,E7,P14);(A1,I3,M4,E7,P15);(A1,I3,M4,E7,P16);(A1,I3,M4,E7,P17);(A1,I3,M4,E7,P18);(A1,I3,M4,E7,P19);(A1,I3,M4,E7,P20);(A1,I3,M4,E8,P1);(A1,I3,M4,E8,P2);(A1,I3,M4,E8,P3);(A1,I3,M4,E8,P4);(A1,I3,M4,E8,P5);(A1,I3,M4,E8,P6);(A1,I3,M4,E8,P7);(A1,I3,M4,E8,P8);(A1,I3,M4,E8,P9);(A1,I3,M4,E8,P10);(A1,I3,M4,E8,P11);(A1,I3,M4,E8,P12);(A1,I3,M4,E8,P13);(A1,I3,M4,E8,P14);(A1,I3,M4,E8,P15);(A1,I3,M4,E8,P16);(A1,I3,M4,E8,P17);(A1,I3,M4,E8,P18);(A1,I3,M4,E8,P19);(A1,I3,M4,E8,P20);(A1,I3,M4,E9,P1);(A1,I3,M4,E9,P2);(A1,I3,M4,E9,P3);(A1,I3,M4,E9,P4);(A1,I3,M4,E9,P5);(A1,I3,M4,E9,P6);(A1,I3,M4,E9,P7);(A1,I3,M4,E9,P8);(A1,I3,M4,E9,P9);(A1,I3,M4,E9,P10);(A1,I3,M4,E9,P11);(A1,I3,M4,E9,P12);(A1,I3,M4,E9,P13);(A1,I3,M4,E9,P14);(A1,I3,M4,E9,P15);(A1,I3,M4,E9,P16);(A1,I3,M4,E9,P17);(A1,I3,M4,E9,P18);(A1,I3,M4,E9,P19);(A1,I3,M4,E9,P20);(A1,I3,M5,E1,P1);(A1,I3,M5,E1,P2);(A1,I3,M5,E1,P3);(A1,I3,M5,E1,P4);(A1,I3,M5,E1,P5);(A1,I3,M5,E1,P6);(A1,I3,M5,E1,P7);(A1,I3,M5,E1,P8);(A1,I3,M5,E1,P9);(A1,I3,M5,E1,P10);(A1,I3,M5,E1,P11);(A1,I3,M5,E1,P12);(A1,I3,M5,E1,P13);(A1,I3,M5,E1,P14);(A1,I3,M5,E1,P15);(A1,I3,M5,E1,P16);(A1,I3,M5,E1,P17);(A1,I3,M5,E1,P18);(A1,I3,M5,E1,P19);(A1,I3,M5,E1,P20);(A1,I3,M5,E2,P1);(A1,I3,M5,E2,P2);(A1,I3,M5,E2,P3);(A1,I3,M5,E2,P4);(A1,I3,M5,E2,P5);(A1,I3,M5,E2,P6);(A1,I3,M5,E2,P7);(A1,I3,M5,E2,P8);(A1,I3,M5,E2,P9);(A1,I3,M5,E2,P10);(A1,I3,M5,E2,P11);(A1,I3,M5,E2,P12);(A1,I3,M5,E2,P13);(A1,I3,M5,E2,P14);(A1,I3,M5,E2,P15);(A1,I3,M5,E2,P16);(A1,I3,M5,E2,P17);(A1,I3,M5,E2,P18);(A1,I3,M5,E2,P19);(A1,I3,M5,E2,P20);(A1,I3,M5,E3,P1);(A1,I3,M5,E3,P2);(A1,I3,M5,E3,P3);(A1,I3,M5,E3,P4);(A1,I3,M5,E3,P5);(A1,I3,M5,E3,P6);(A1,I3,M5,E3,P7);(A1,I3,M5,E3,P8);(A1,I3,M5,E3,P9);(A1,I3,M5,E3,P10);(A1,I3,M5,E3,P11);(A1,I3,M5,E3,P12);(A1,I3,M5,E3,P13);(A1,I3,M5,E3,P14);(A1,I3,M5,E3,P15);(A1,I3,M5,E3,P16);(A1,I3,M5,E3,P17);(A1,I3,M5,E3,P18);(A1,I3,M5,E3,P19);(A1,I3,M5,E3,P20);(A1,I3,M5,E4,P1);(A1,I3,M5,E4,P2);(A1,I3,M5,E4,P3);(A1,I3,M5,E4,P4);(A1,I3,M5,E4,P5);(A1,I3,M5,E4,P6);(A1,I3,M5,E4,P7);(A1,I3,M5,E4,P8);(A1,I3,M5,E4,P9);(A1,I3,M5,E4,P10);(A1,I3,M5,E4,P11);(A1,I3,M5,E4,P12);(A1,I3,M5,E4,P13);(A1,I3,M5,E4,P14);(A1,I3,M5,E4,P15);(A1,I3,M5,E4,P16);(A1,I3,M5,E4,P17);(A1,I3,M5,E4,P18);(A1,I3,M5,E4,P19);(A1,I3,M5,E4,P20);(A1,I3,M5,E5,P1);(A1,I3,M5,E5,P2);(A1,I3,M5,E5,P3);(A1,I3,M5,E5,P4);(A1,I3,M5,E5,P5);(A1,I3,M5,E5,P6);(A1,I3,M5,E5,P7);(A1,I3,M5,E5,P8);(A1,I3,M5,E5,P9);(A1,I3,M5,E5,P10);(A1,I3,M5,E5,P11);(A1,I3,M5,E5,P12);(A1,I3,M5,E5,P13);(A1,I3,M5,E5,P14);(A1,I3,M5,E5,P15);(A1,I3,M5,E5,P16);(A1,I3,M5,E5,P17);(A1,I3,M5,E5,P18);(A1,I3,M5,E5,P19);(A1,I3,M5,E5,P20);(A1,I3,M5,E6,P1);(A1,I3,M5,E6,P2);(A1,I3,M5,E6,P3);(A1,I3,M5,E6,P4);(A1,I3,M5,E6,P5);(A1,I3,M5,E6,P6);(A1,I3,M5,E6,P7);(A1,I3,M5,E6,P8);(A1,I3,M5,E6,P9);(A1,I3,M5,E6,P10);(A1,I3,M5,E6,P11);(A1,I3,M5,E6,P12);(A1,I3,M5,E6,P13);(A1,I3,M5,E6,P14);(A1,I3,M5,E6,P15);(A1,I3,M5,E6,P16);(A1,I3,M5,E6,P17);(A1,I3,M5,E6,P18);(A1,I3,M5,E6,P19);(A1,I3,M5,E6,P20);(A1,I3,M5,E7,P1);(A1,I3,M5,E7,P2);(A1,I3,M5,E7,P3);(A1,I3,M5,E7,P4);(A1,I3,M5,E7,P5);(A1,I3,M5,E7,P6);(A1,I3,M5,E7,P7);(A1,I3,M5,E7,P8);(A1,I3,M5,E7,P9);(A1,I3,M5,E7,P10);(A1,I3,M5,E7,P11);(A1,I3,M5,E7,P12);(A1,I3,M5,E7,P13);(A1,I3,M5,E7,P14);(A1,I3,M5,E7,P15);(A1,I3,M5,E7,P16);(A1,I3,M5,E7,P17);(A1,I3,M5,E7,P18);(A1,I3,M5,E7,P19);(A1,I3,M5,E7,P20);(A1,I3,M5,E8,P1);(A1,I3,M5,E8,P2);(A1,I3,M5,E8,P3);(A1,I3,M5,E8,P4);(A1,I3,M5,E8,P5);(A1,I3,M5,E8,P6);(A1,I3,M5,E8,P7);(A1,I3,M5,E8,P8);(A1,I3,M5,E8,P9);(A1,I3,M5,E8,P10);(A1,I3,M5,E8,P11);(A1,I3,M5,E8,P12);(A1,I3,M5,E8,P13);(A1,I3,M5,E8,P14);(A1,I3,M5,E8,P15);(A1,I3,M5,E8,P16);(A1,I3,M5,E8,P17);(A1,I3,M5,E8,P18);(A1,I3,M5,E8,P19);(A1,I3,M5,E8,P20);(A1,I3,M5,E9,P1);(A1,I3,M5,E9,P2);(A1,I3,M5,E9,P3);(A1,I3,M5,E9,P4);(A1,I3,M5,E9,P5);(A1,I3,M5,E9,P6);(A1,I3,M5,E9,P7);(A1,I3,M5,E9,P8);(A1,I3,M5,E9,P9);(A1,I3,M5,E9,P10);(A1,I3,M5,E9,P11);(A1,I3,M5,E9,P12);(A1,I3,M5,E9,P13);(A1,I3,M5,E9,P14);(A1,I3,M5,E9,P15);(A1,I3,M5,E9,P16);(A1,I3,M5,E9,P17);(A1,I3,M5,E9,P18);(A1,I3,M5,E9,P19);(A1,I3,M5,E9,P20);(A1,I3,M6,E1,P1);(A1,I3,M6,E1,P2);(A1,I3,M6,E1,P3);(A1,I3,M6,E1,P4);(A1,I3,M6,E1,P5);(A1,I3,M6,E1,P6);(A1,I3,M6,E1,P7);(A1,I3,M6,E1,P8);(A1,I3,M6,E1,P9);(A1,I3,M6,E1,P10);(A1,I3,M6,E1,P11);(A1,I3,M6,E1,P12);(A1,I3,M6,E1,P13);(A1,I3,M6,E1,P14);(A1,I3,M6,E1,P15);(A1,I3,M6,E1,P16);(A1,I3,M6,E1,P17);(A1,I3,M6,E1,P18);(A1,I3,M6,E1,P19);(A1,I3,M6,E1,P20);(A1,I3,M6,E2,P1);(A1,I3,M6,E2,P2);(A1,I3,M6,E2,P3);(A1,I3,M6,E2,P4);(A1,I3,M6,E2,P5);(A1,I3,M6,E2,P6);(A1,I3,M6,E2,P7);(A1,I3,M6,E2,P8);(A1,I3,M6,E2,P9);(A1,I3,M6,E2,P10);(A1,I3,M6,E2,P11);(A1,I3,M6,E2,P12);(A1,I3,M6,E2,P13);(A1,I3,M6,E2,P14);(A1,I3,M6,E2,P15);(A1,I3,M6,E2,P16);(A1,I3,M6,E2,P17);(A1,I3,M6,E2,P18);(A1,I3,M6,E2,P19);(A1,I3,M6,E2,P20);(A1,I3,M6,E3,P1);(A1,I3,M6,E3,P2);(A1,I3,M6,E3,P3);(A1,I3,M6,E3,P4);(A1,I3,M6,E3,P5);(A1,I3,M6,E3,P6);(A1,I3,M6,E3,P7);(A1,I3,M6,E3,P8);(A1,I3,M6,E3,P9);(A1,I3,M6,E3,P10);(A1,I3,M6,E3,P11);(A1,I3,M6,E3,P12);(A1,I3,M6,E3,P13);(A1,I3,M6,E3,P14);(A1,I3,M6,E3,P15);(A1,I3,M6,E3,P16);(A1,I3,M6,E3,P17);(A1,I3,M6,E3,P18);(A1,I3,M6,E3,P19);(A1,I3,M6,E3,P20);(A1,I3,M6,E4,P1);(A1,I3,M6,E4,P2);(A1,I3,M6,E4,P3);(A1,I3,M6,E4,P4);(A1,I3,M6,E4,P5);(A1,I3,M6,E4,P6);(A1,I3,M6,E4,P7);(A1,I3,M6,E4,P8);(A1,I3,M6,E4,P9);(A1,I3,M6,E4,P10);(A1,I3,M6,E4,P11);(A1,I3,M6,E4,P12);(A1,I3,M6,E4,P13);(A1,I3,M6,E4,P14);(A1,I3,M6,E4,P15);(A1,I3,M6,E4,P16);(A1,I3,M6,E4,P17);(A1,I3,M6,E4,P18);(A1,I3,M6,E4,P19);(A1,I3,M6,E4,P20);(A1,I3,M6,E5,P1);(A1,I3,M6,E5,P2);(A1,I3,M6,E5,P3);(A1,I3,M6,E5,P4);(A1,I3,M6,E5,P5);(A1,I3,M6,E5,P6);(A1,I3,M6,E5,P7);(A1,I3,M6,E5,P8);(A1,I3,M6,E5,P9);(A1,I3,M6,E5,P10);(A1,I3,M6,E5,P11);(A1,I3,M6,E5,P12);(A1,I3,M6,E5,P13);(A1,I3,M6,E5,P14);(A1,I3,M6,E5,P15);(A1,I3,M6,E5,P16);(A1,I3,M6,E5,P17);(A1,I3,M6,E5,P18);(A1,I3,M6,E5,P19);(A1,I3,M6,E5,P20);(A1,I3,M6,E6,P1);(A1,I3,M6,E6,P2);(A1,I3,M6,E6,P3);(A1,I3,M6,E6,P4);(A1,I3,M6,E6,P5);(A1,I3,M6,E6,P6);(A1,I3,M6,E6,P7);(A1,I3,M6,E6,P8);(A1,I3,M6,E6,P9);(A1,I3,M6,E6,P10);(A1,I3,M6,E6,P11);(A1,I3,M6,E6,P12);(A1,I3,M6,E6,P13);(A1,I3,M6,E6,P14);(A1,I3,M6,E6,P15);(A1,I3,M6,E6,P16);(A1,I3,M6,E6,P17);(A1,I3,M6,E6,P18);(A1,I3,M6,E6,P19);(A1,I3,M6,E6,P20);(A1,I3,M6,E7,P1);(A1,I3,M6,E7,P2);(A1,I3,M6,E7,P3);(A1,I3,M6,E7,P4);(A1,I3,M6,E7,P5);(A1,I3,M6,E7,P6);(A1,I3,M6,E7,P7);(A1,I3,M6,E7,P8);(A1,I3,M6,E7,P9);(A1,I3,M6,E7,P10);(A1,I3,M6,E7,P11);(A1,I3,M6,E7,P12);(A1,I3,M6,E7,P13);(A1,I3,M6,E7,P14);(A1,I3,M6,E7,P15);(A1,I3,M6,E7,P16);(A1,I3,M6,E7,P17);(A1,I3,M6,E7,P18);(A1,I3,M6,E7,P19);(A1,I3,M6,E7,P20);(A1,I3,M6,E8,P1);(A1,I3,M6,E8,P2);(A1,I3,M6,E8,P3);(A1,I3,M6,E8,P4);(A1,I3,M6,E8,P5);(A1,I3,M6,E8,P6);(A1,I3,M6,E8,P7);(A1,I3,M6,E8,P8);(A1,I3,M6,E8,P9);(A1,I3,M6,E8,P10);(A1,I3,M6,E8,P11);(A1,I3,M6,E8,P12);(A1,I3,M6,E8,P13);(A1,I3,M6,E8,P14);(A1,I3,M6,E8,P15);(A1,I3,M6,E8,P16);(A1,I3,M6,E8,P17);(A1,I3,M6,E8,P18);(A1,I3,M6,E8,P19);(A1,I3,M6,E8,P20);(A1,I3,M6,E9,P1);(A1,I3,M6,E9,P2);(A1,I3,M6,E9,P3);(A1,I3,M6,E9,P4);(A1,I3,M6,E9,P5);(A1,I3,M6,E9,P6);(A1,I3,M6,E9,P7);(A1,I3,M6,E9,P8);(A1,I3,M6,E9,P9);(A1,I3,M6,E9,P10);(A1,I3,M6,E9,P11);(A1,I3,M6,E9,P12);(A1,I3,M6,E9,P13);(A1,I3,M6,E9,P14);(A1,I3,M6,E9,P15);(A1,I3,M6,E9,P16);(A1,I3,M6,E9,P17);(A1,I3,M6,E9,P18);(A1,I3,M6,E9,P19);(A1,I3,M6,E9,P20);(A1,I4,M1,E1,P1);(A1,I4,M1,E1,P2);(A1,I4,M1,E1,P3);(A1,I4,M1,E1,P4);(A1,I4,M1,E1,P5);(A1,I4,M1,E1,P6);(A1,I4,M1,E1,P7);(A1,I4,M1,E1,P8);(A1,I4,M1,E1,P9);(A1,I4,M1,E1,P10);(A1,I4,M1,E1,P11);(A1,I4,M1,E1,P12);(A1,I4,M1,E1,P13);(A1,I4,M1,E1,P14);(A1,I4,M1,E1,P15);(A1,I4,M1,E1,P16);(A1,I4,M1,E1,P17);(A1,I4,M1,E1,P18);(A1,I4,M1,E1,P19);(A1,I4,M1,E1,P20);(A1,I4,M1,E2,P1);(A1,I4,M1,E2,P2);(A1,I4,M1,E2,P3);(A1,I4,M1,E2,P4);(A1,I4,M1,E2,P5);(A1,I4,M1,E2,P6);(A1,I4,M1,E2,P7);(A1,I4,M1,E2,P8);(A1,I4,M1,E2,P9);(A1,I4,M1,E2,P10);(A1,I4,M1,E2,P11);(A1,I4,M1,E2,P12);(A1,I4,M1,E2,P13);(A1,I4,M1,E2,P14);(A1,I4,M1,E2,P15);(A1,I4,M1,E2,P16);(A1,I4,M1,E2,P17);(A1,I4,M1,E2,P18);(A1,I4,M1,E2,P19);(A1,I4,M1,E2,P20);(A1,I4,M1,E3,P1);(A1,I4,M1,E3,P2);(A1,I4,M1,E3,P3);(A1,I4,M1,E3,P4);(A1,I4,M1,E3,P5);(A1,I4,M1,E3,P6);(A1,I4,M1,E3,P7);(A1,I4,M1,E3,P8);(A1,I4,M1,E3,P9);(A1,I4,M1,E3,P10);(A1,I4,M1,E3,P11);(A1,I4,M1,E3,P12);(A1,I4,M1,E3,P13);(A1,I4,M1,E3,P14);(A1,I4,M1,E3,P15);(A1,I4,M1,E3,P16);(A1,I4,M1,E3,P17);(A1,I4,M1,E3,P18);(A1,I4,M1,E3,P19);(A1,I4,M1,E3,P20);(A1,I4,M1,E4,P1);(A1,I4,M1,E4,P2);(A1,I4,M1,E4,P3);(A1,I4,M1,E4,P4);(A1,I4,M1,E4,P5);(A1,I4,M1,E4,P6);(A1,I4,M1,E4,P7);(A1,I4,M1,E4,P8);(A1,I4,M1,E4,P9);(A1,I4,M1,E4,P10);(A1,I4,M1,E4,P11);(A1,I4,M1,E4,P12);(A1,I4,M1,E4,P13);(A1,I4,M1,E4,P14);(A1,I4,M1,E4,P15);(A1,I4,M1,E4,P16);(A1,I4,M1,E4,P17);(A1,I4,M1,E4,P18);(A1,I4,M1,E4,P19);(A1,I4,M1,E4,P20);(A1,I4,M1,E5,P1);(A1,I4,M1,E5,P2);(A1,I4,M1,E5,P3);(A1,I4,M1,E5,P4);(A1,I4,M1,E5,P5);(A1,I4,M1,E5,P6);(A1,I4,M1,E5,P7);(A1,I4,M1,E5,P8);(A1,I4,M1,E5,P9);(A1,I4,M1,E5,P10);(A1,I4,M1,E5,P11);(A1,I4,M1,E5,P12);(A1,I4,M1,E5,P13);(A1,I4,M1,E5,P14);(A1,I4,M1,E5,P15);(A1,I4,M1,E5,P16);(A1,I4,M1,E5,P17);(A1,I4,M1,E5,P18);(A1,I4,M1,E5,P19);(A1,I4,M1,E5,P20);(A1,I4,M1,E6,P1);(A1,I4,M1,E6,P2);(A1,I4,M1,E6,P3);(A1,I4,M1,E6,P4);(A1,I4,M1,E6,P5);(A1,I4,M1,E6,P6);(A1,I4,M1,E6,P7);(A1,I4,M1,E6,P8);(A1,I4,M1,E6,P9);(A1,I4,M1,E6,P10);(A1,I4,M1,E6,P11);(A1,I4,M1,E6,P12);(A1,I4,M1,E6,P13);(A1,I4,M1,E6,P14);(A1,I4,M1,E6,P15);(A1,I4,M1,E6,P16);(A1,I4,M1,E6,P17);(A1,I4,M1,E6,P18);(A1,I4,M1,E6,P19);(A1,I4,M1,E6,P20);(A1,I4,M1,E7,P1);(A1,I4,M1,E7,P2);(A1,I4,M1,E7,P3);(A1,I4,M1,E7,P4);(A1,I4,M1,E7,P5);(A1,I4,M1,E7,P6);(A1,I4,M1,E7,P7);(A1,I4,M1,E7,P8);(A1,I4,M1,E7,P9);(A1,I4,M1,E7,P10);(A1,I4,M1,E7,P11);(A1,I4,M1,E7,P12);(A1,I4,M1,E7,P13);(A1,I4,M1,E7,P14);(A1,I4,M1,E7,P15);(A1,I4,M1,E7,P16);(A1,I4,M1,E7,P17);(A1,I4,M1,E7,P18);(A1,I4,M1,E7,P19);(A1,I4,M1,E7,P20);(A1,I4,M1,E8,P1);(A1,I4,M1,E8,P2);(A1,I4,M1,E8,P3);(A1,I4,M1,E8,P4);(A1,I4,M1,E8,P5);(A1,I4,M1,E8,P6);(A1,I4,M1,E8,P7);(A1,I4,M1,E8,P8);(A1,I4,M1,E8,P9);(A1,I4,M1,E8,P10);(A1,I4,M1,E8,P11);(A1,I4,M1,E8,P12);(A1,I4,M1,E8,P13);(A1,I4,M1,E8,P14);(A1,I4,M1,E8,P15);(A1,I4,M1,E8,P16);(A1,I4,M1,E8,P17);(A1,I4,M1,E8,P18);(A1,I4,M1,E8,P19);(A1,I4,M1,E8,P20);(A1,I4,M1,E9,P1);(A1,I4,M1,E9,P2);(A1,I4,M1,E9,P3);(A1,I4,M1,E9,P4);(A1,I4,M1,E9,P5);(A1,I4,M1,E9,P6);(A1,I4,M1,E9,P7);(A1,I4,M1,E9,P8);(A1,I4,M1,E9,P9);(A1,I4,M1,E9,P10);(A1,I4,M1,E9,P11);(A1,I4,M1,E9,P12);(A1,I4,M1,E9,P13);(A1,I4,M1,E9,P14);(A1,I4,M1,E9,P15);(A1,I4,M1,E9,P16);(A1,I4,M1,E9,P17);(A1,I4,M1,E9,P18);(A1,I4,M1,E9,P19);(A1,I4,M1,E9,P20);(A1,I4,M2,E1,P1);(A1,I4,M2,E1,P2);(A1,I4,M2,E1,P3);(A1,I4,M2,E1,P4);(A1,I4,M2,E1,P5);(A1,I4,M2,E1,P6);(A1,I4,M2,E1,P7);(A1,I4,M2,E1,P8);(A1,I4,M2,E1,P9);(A1,I4,M2,E1,P10);(A1,I4,M2,E1,P11);(A1,I4,M2,E1,P12);(A1,I4,M2,E1,P13);(A1,I4,M2,E1,P14);(A1,I4,M2,E1,P15);(A1,I4,M2,E1,P16);(A1,I4,M2,E1,P17);(A1,I4,M2,E1,P18);(A1,I4,M2,E1,P19);(A1,I4,M2,E1,P20);(A1,I4,M2,E2,P1);(A1,I4,M2,E2,P2);(A1,I4,M2,E2,P3);(A1,I4,M2,E2,P4);(A1,I4,M2,E2,P5);(A1,I4,M2,E2,P6);(A1,I4,M2,E2,P7);(A1,I4,M2,E2,P8);(A1,I4,M2,E2,P9);(A1,I4,M2,E2,P10);(A1,I4,M2,E2,P11);(A1,I4,M2,E2,P12);(A1,I4,M2,E2,P13);(A1,I4,M2,E2,P14);(A1,I4,M2,E2,P15);(A1,I4,M2,E2,P16);(A1,I4,M2,E2,P17);(A1,I4,M2,E2,P18);(A1,I4,M2,E2,P19);(A1,I4,M2,E2,P20);(A1,I4,M2,E3,P1);(A1,I4,M2,E3,P2);(A1,I4,M2,E3,P3);(A1,I4,M2,E3,P4);(A1,I4,M2,E3,P5);(A1,I4,M2,E3,P6);(A1,I4,M2,E3,P7);(A1,I4,M2,E3,P8);(A1,I4,M2,E3,P9);(A1,I4,M2,E3,P10);(A1,I4,M2,E3,P11);(A1,I4,M2,E3,P12);(A1,I4,M2,E3,P13);(A1,I4,M2,E3,P14);(A1,I4,M2,E3,P15);(A1,I4,M2,E3,P16);(A1,I4,M2,E3,P17);(A1,I4,M2,E3,P18);(A1,I4,M2,E3,P19);(A1,I4,M2,E3,P20);(A1,I4,M2,E4,P1);(A1,I4,M2,E4,P2);(A1,I4,M2,E4,P3);(A1,I4,M2,E4,P4);(A1,I4,M2,E4,P5);(A1,I4,M2,E4,P6);(A1,I4,M2,E4,P7);(A1,I4,M2,E4,P8);(A1,I4,M2,E4,P9);(A1,I4,M2,E4,P10);(A1,I4,M2,E4,P11);(A1,I4,M2,E4,P12);(A1,I4,M2,E4,P13);(A1,I4,M2,E4,P14);(A1,I4,M2,E4,P15);(A1,I4,M2,E4,P16);(A1,I4,M2,E4,P17);(A1,I4,M2,E4,P18);(A1,I4,M2,E4,P19);(A1,I4,M2,E4,P20);(A1,I4,M2,E5,P1);(A1,I4,M2,E5,P2);(A1,I4,M2,E5,P3);(A1,I4,M2,E5,P4);(A1,I4,M2,E5,P5);(A1,I4,M2,E5,P6);(A1,I4,M2,E5,P7);(A1,I4,M2,E5,P8);(A1,I4,M2,E5,P9);(A1,I4,M2,E5,P10);(A1,I4,M2,E5,P11);(A1,I4,M2,E5,P12);(A1,I4,M2,E5,P13);(A1,I4,M2,E5,P14);(A1,I4,M2,E5,P15);(A1,I4,M2,E5,P16);(A1,I4,M2,E5,P17);(A1,I4,M2,E5,P18);(A1,I4,M2,E5,P19);(A1,I4,M2,E5,P20);(A1,I4,M2,E6,P1);(A1,I4,M2,E6,P2);(A1,I4,M2,E6,P3);(A1,I4,M2,E6,P4);(A1,I4,M2,E6,P5);(A1,I4,M2,E6,P6);(A1,I4,M2,E6,P7);(A1,I4,M2,E6,P8);(A1,I4,M2,E6,P9);(A1,I4,M2,E6,P10);(A1,I4,M2,E6,P11);(A1,I4,M2,E6,P12);(A1,I4,M2,E6,P13);(A1,I4,M2,E6,P14);(A1,I4,M2,E6,P15);(A1,I4,M2,E6,P16);(A1,I4,M2,E6,P17);(A1,I4,M2,E6,P18);(A1,I4,M2,E6,P19);(A1,I4,M2,E6,P20);(A1,I4,M2,E7,P1);(A1,I4,M2,E7,P2);(A1,I4,M2,E7,P3);(A1,I4,M2,E7,P4);(A1,I4,M2,E7,P5);(A1,I4,M2,E7,P6);(A1,I4,M2,E7,P7);(A1,I4,M2,E7,P8);(A1,I4,M2,E7,P9);(A1,I4,M2,E7,P10);(A1,I4,M2,E7,P11);(A1,I4,M2,E7,P12);(A1,I4,M2,E7,P13);(A1,I4,M2,E7,P14);(A1,I4,M2,E7,P15);(A1,I4,M2,E7,P16);(A1,I4,M2,E7,P17);(A1,I4,M2,E7,P18);(A1,I4,M2,E7,P19);(A1,I4,M2,E7,P20);(A1,I4,M2,E8,P1);(A1,I4,M2,E8,P2);(A1,I4,M2,E8,P3);(A1,I4,M2,E8,P4);(A1,I4,M2,E8,P5);(A1,I4,M2,E8,P6);(A1,I4,M2,E8,P7);(A1,I4,M2,E8,P8);(A1,I4,M2,E8,P9);(A1,I4,M2,E8,P10);(A1,I4,M2,E8,P11);(A1,I4,M2,E8,P12);(A1,I4,M2,E8,P13);(A1,I4,M2,E8,P14);(A1,I4,M2,E8,P15);(A1,I4,M2,E8,P16);(A1,I4,M2,E8,P17);(A1,I4,M2,E8,P18);(A1,I4,M2,E8,P19);(A1,I4,M2,E8,P20);(A1,I4,M2,E9,P1);(A1,I4,M2,E9,P2);(A1,I4,M2,E9,P3);(A1,I4,M2,E9,P4);(A1,I4,M2,E9,P5);(A1,I4,M2,E9,P6);(A1,I4,M2,E9,P7);(A1,I4,M2,E9,P8);(A1,I4,M2,E9,P9);(A1,I4,M2,E9,P10);(A1,I4,M2,E9,P11);(A1,I4,M2,E9,P12);(A1,I4,M2,E9,P13);(A1,I4,M2,E9,P14);(A1,I4,M2,E9,P15);(A1,I4,M2,E9,P16);(A1,I4,M2,E9,P17);(A1,I4,M2,E9,P18);(A1,I4,M2,E9,P19);(A1,I4,M2,E9,P20);(A1,I4,M3,E1,P1);(A1,I4,M3,E1,P2);(A1,I4,M3,E1,P3);(A1,I4,M3,E1,P4);(A1,I4,M3,E1,P5);(A1,I4,M3,E1,P6);(A1,I4,M3,E1,P7);(A1,I4,M3,E1,P8);(A1,I4,M3,E1,P9);(A1,I4,M3,E1,P10);(A1,I4,M3,E1,P11);(A1,I4,M3,E1,P12);(A1,I4,M3,E1,P13);(A1,I4,M3,E1,P14);(A1,I4,M3,E1,P15);(A1,I4,M3,E1,P16);(A1,I4,M3,E1,P17);(A1,I4,M3,E1,P18);(A1,I4,M3,E1,P19);(A1,I4,M3,E1,P20);(A1,I4,M3,E2,P1);(A1,I4,M3,E2,P2);(A1,I4,M3,E2,P3);(A1,I4,M3,E2,P4);(A1,I4,M3,E2,P5);(A1,I4,M3,E2,P6);(A1,I4,M3,E2,P7);(A1,I4,M3,E2,P8);(A1,I4,M3,E2,P9);(A1,I4,M3,E2,P10);(A1,I4,M3,E2,P11);(A1,I4,M3,E2,P12);(A1,I4,M3,E2,P13);(A1,I4,M3,E2,P14);(A1,I4,M3,E2,P15);(A1,I4,M3,E2,P16);(A1,I4,M3,E2,P17);(A1,I4,M3,E2,P18);(A1,I4,M3,E2,P19);(A1,I4,M3,E2,P20);(A1,I4,M3,E3,P1);(A1,I4,M3,E3,P2);(A1,I4,M3,E3,P3);(A1,I4,M3,E3,P4);(A1,I4,M3,E3,P5);(A1,I4,M3,E3,P6);(A1,I4,M3,E3,P7);(A1,I4,M3,E3,P8);(A1,I4,M3,E3,P9);(A1,I4,M3,E3,P10);(A1,I4,M3,E3,P11);(A1,I4,M3,E3,P12);(A1,I4,M3,E3,P13);(A1,I4,M3,E3,P14);(A1,I4,M3,E3,P15);(A1,I4,M3,E3,P16);(A1,I4,M3,E3,P17);(A1,I4,M3,E3,P18);(A1,I4,M3,E3,P19);(A1,I4,M3,E3,P20);(A1,I4,M3,E4,P1);(A1,I4,M3,E4,P2);(A1,I4,M3,E4,P3);(A1,I4,M3,E4,P4);(A1,I4,M3,E4,P5);(A1,I4,M3,E4,P6);(A1,I4,M3,E4,P7);(A1,I4,M3,E4,P8);(A1,I4,M3,E4,P9);(A1,I4,M3,E4,P10);(A1,I4,M3,E4,P11);(A1,I4,M3,E4,P12);(A1,I4,M3,E4,P13);(A1,I4,M3,E4,P14);(A1,I4,M3,E4,P15);(A1,I4,M3,E4,P16);(A1,I4,M3,E4,P17);(A1,I4,M3,E4,P18);(A1,I4,M3,E4,P19);(A1,I4,M3,E4,P20);(A1,I4,M3,E5,P1);(A1,I4,M3,E5,P2);(A1,I4,M3,E5,P3);(A1,I4,M3,E5,P4);(A1,I4,M3,E5,P5);(A1,I4,M3,E5,P6);(A1,I4,M3,E5,P7);(A1,I4,M3,E5,P8);(A1,I4,M3,E5,P9);(A1,I4,M3,E5,P10);(A1,I4,M3,E5,P11);(A1,I4,M3,E5,P12);(A1,I4,M3,E5,P13);(A1,I4,M3,E5,P14);(A1,I4,M3,E5,P15);(A1,I4,M3,E5,P16);(A1,I4,M3,E5,P17);(A1,I4,M3,E5,P18);(A1,I4,M3,E5,P19);(A1,I4,M3,E5,P20);(A1,I4,M3,E6,P1);(A1,I4,M3,E6,P2);(A1,I4,M3,E6,P3);(A1,I4,M3,E6,P4);(A1,I4,M3,E6,P5);(A1,I4,M3,E6,P6);(A1,I4,M3,E6,P7);(A1,I4,M3,E6,P8);(A1,I4,M3,E6,P9);(A1,I4,M3,E6,P10);(A1,I4,M3,E6,P11);(A1,I4,M3,E6,P12);(A1,I4,M3,E6,P13);(A1,I4,M3,E6,P14);(A1,I4,M3,E6,P15);(A1,I4,M3,E6,P16);(A1,I4,M3,E6,P17);(A1,I4,M3,E6,P18);(A1,I4,M3,E6,P19);(A1,I4,M3,E6,P20);(A1,I4,M3,E7,P1);(A1,I4,M3,E7,P2);(A1,I4,M3,E7,P3);(A1,I4,M3,E7,P4);(A1,I4,M3,E7,P5);(A1,I4,M3,E7,P6);(A1,I4,M3,E7,P7);(A1,I4,M3,E7,P8);(A1,I4,M3,E7,P9);(A1,I4,M3,E7,P10);(A1,I4,M3,E7,P11);(A1,I4,M3,E7,P12);(A1,I4,M3,E7,P13);(A1,I4,M3,E7,P14);(A1,I4,M3,E7,P15);(A1,I4,M3,E7,P16);(A1,I4,M3,E7,P17);(A1,I4,M3,E7,P18);(A1,I4,M3,E7,P19);(A1,I4,M3,E7,P20);(A1,I4,M3,E8,P1);(A1,I4,M3,E8,P2);(A1,I4,M3,E8,P3);(A1,I4,M3,E8,P4);(A1,I4,M3,E8,P5);(A1,I4,M3,E8,P6);(A1,I4,M3,E8,P7);(A1,I4,M3,E8,P8);(A1,I4,M3,E8,P9);(A1,I4,M3,E8,P10);(A1,I4,M3,E8,P11);(A1,I4,M3,E8,P12);(A1,I4,M3,E8,P13);(A1,I4,M3,E8,P14);(A1,I4,M3,E8,P15);(A1,I4,M3,E8,P16);(A1,I4,M3,E8,P17);(A1,I4,M3,E8,P18);(A1,I4,M3,E8,P19);(A1,I4,M3,E8,P20);(A1,I4,M3,E9,P1);(A1,I4,M3,E9,P2);(A1,I4,M3,E9,P3);(A1,I4,M3,E9,P4);(A1,I4,M3,E9,P5);(A1,I4,M3,E9,P6);(A1,I4,M3,E9,P7);(A1,I4,M3,E9,P8);(A1,I4,M3,E9,P9);(A1,I4,M3,E9,P10);(A1,I4,M3,E9,P11);(A1,I4,M3,E9,P12);(A1,I4,M3,E9,P13);(A1,I4,M3,E9,P14);(A1,I4,M3,E9,P15);(A1,I4,M3,E9,P16);(A1,I4,M3,E9,P17);(A1,I4,M3,E9,P18);(A1,I4,M3,E9,P19);(A1,I4,M3,E9,P20);(A1,I4,M4,E1,P1);(A1,I4,M4,E1,P2);(A1,I4,M4,E1,P3);(A1,I4,M4,E1,P4);(A1,I4,M4,E1,P5);(A1,I4,M4,E1,P6);(A1,I4,M4,E1,P7);(A1,I4,M4,E1,P8);(A1,I4,M4,E1,P9);(A1,I4,M4,E1,P10);(A1,I4,M4,E1,P11);(A1,I4,M4,E1,P12);(A1,I4,M4,E1,P13);(A1,I4,M4,E1,P14);(A1,I4,M4,E1,P15);(A1,I4,M4,E1,P16);(A1,I4,M4,E1,P17);(A1,I4,M4,E1,P18);(A1,I4,M4,E1,P19);(A1,I4,M4,E1,P20);(A1,I4,M4,E2,P1);(A1,I4,M4,E2,P2);(A1,I4,M4,E2,P3);(A1,I4,M4,E2,P4);(A1,I4,M4,E2,P5);(A1,I4,M4,E2,P6);(A1,I4,M4,E2,P7);(A1,I4,M4,E2,P8);(A1,I4,M4,E2,P9);(A1,I4,M4,E2,P10);(A1,I4,M4,E2,P11);(A1,I4,M4,E2,P12);(A1,I4,M4,E2,P13);(A1,I4,M4,E2,P14);(A1,I4,M4,E2,P15);(A1,I4,M4,E2,P16);(A1,I4,M4,E2,P17);(A1,I4,M4,E2,P18);(A1,I4,M4,E2,P19);(A1,I4,M4,E2,P20);(A1,I4,M4,E3,P1);(A1,I4,M4,E3,P2);(A1,I4,M4,E3,P3);(A1,I4,M4,E3,P4);(A1,I4,M4,E3,P5);(A1,I4,M4,E3,P6);(A1,I4,M4,E3,P7);(A1,I4,M4,E3,P8);(A1,I4,M4,E3,P9);(A1,I4,M4,E3,P10);(A1,I4,M4,E3,P11);(A1,I4,M4,E3,P12);(A1,I4,M4,E3,P13);(A1,I4,M4,E3,P14);(A1,I4,M4,E3,P15);(A1,I4,M4,E3,P16);(A1,I4,M4,E3,P17);(A1,I4,M4,E3,P18);(A1,I4,M4,E3,P19);(A1,I4,M4,E3,P20);(A1,I4,M4,E4,P1);(A1,I4,M4,E4,P2);(A1,I4,M4,E4,P3);(A1,I4,M4,E4,P4);(A1,I4,M4,E4,P5);(A1,I4,M4,E4,P6);(A1,I4,M4,E4,P7);(A1,I4,M4,E4,P8);(A1,I4,M4,E4,P9);(A1,I4,M4,E4,P10);(A1,I4,M4,E4,P11);(A1,I4,M4,E4,P12);(A1,I4,M4,E4,P13);(A1,I4,M4,E4,P14);(A1,I4,M4,E4,P15);(A1,I4,M4,E4,P16);(A1,I4,M4,E4,P17);(A1,I4,M4,E4,P18);(A1,I4,M4,E4,P19);(A1,I4,M4,E4,P20);(A1,I4,M4,E5,P1);(A1,I4,M4,E5,P2);(A1,I4,M4,E5,P3);(A1,I4,M4,E5,P4);(A1,I4,M4,E5,P5);(A1,I4,M4,E5,P6);(A1,I4,M4,E5,P7);(A1,I4,M4,E5,P8);(A1,I4,M4,E5,P9);(A1,I4,M4,E5,P10);(A1,I4,M4,E5,P11);(A1,I4,M4,E5,P12);(A1,I4,M4,E5,P13);(A1,I4,M4,E5,P14);(A1,I4,M4,E5,P15);(A1,I4,M4,E5,P16);(A1,I4,M4,E5,P17);(A1,I4,M4,E5,P18);(A1,I4,M4,E5,P19);(A1,I4,M4,E5,P20);(A1,I4,M4,E6,P1);(A1,I4,M4,E6,P2);(A1,I4,M4,E6,P3);(A1,I4,M4,E6,P4);(A1,I4,M4,E6,P5);(A1,I4,M4,E6,P6);(A1,I4,M4,E6,P7);(A1,I4,M4,E6,P8);(A1,I4,M4,E6,P9);(A1,I4,M4,E6,P10);(A1,I4,M4,E6,P11);(A1,I4,M4,E6,P12);(A1,I4,M4,E6,P13);(A1,I4,M4,E6,P14);(A1,I4,M4,E6,P15);(A1,I4,M4,E6,P16);(A1,I4,M4,E6,P17);(A1,I4,M4,E6,P18);(A1,I4,M4,E6,P19);(A1,I4,M4,E6,P20);(A1,I4,M4,E7,P1);(A1,I4,M4,E7,P2);(A1,I4,M4,E7,P3);(A1,I4,M4,E7,P4);(A1,I4,M4,E7,P5);(A1,I4,M4,E7,P6);(A1,I4,M4,E7,P7);(A1,I4,M4,E7,P8);(A1,I4,M4,E7,P9);(A1,I4,M4,E7,P10);(A1,I4,M4,E7,P11);(A1,I4,M4,E7,P12);(A1,I4,M4,E7,P13);(A1,I4,M4,E7,P14);(A1,I4,M4,E7,P15);(A1,I4,M4,E7,P16);(A1,I4,M4,E7,P17);(A1,I4,M4,E7,P18);(A1,I4,M4,E7,P19);(A1,I4,M4,E7,P20);(A1,I4,M4,E8,P1);(A1,I4,M4,E8,P2);(A1,I4,M4,E8,P3);(A1,I4,M4,E8,P4);(A1,I4,M4,E8,P5);(A1,I4,M4,E8,P6);(A1,I4,M4,E8,P7);(A1,I4,M4,E8,P8);(A1,I4,M4,E8,P9);(A1,I4,M4,E8,P10);(A1,I4,M4,E8,P11);(A1,I4,M4,E8,P12);(A1,I4,M4,E8,P13);(A1,I4,M4,E8,P14);(A1,I4,M4,E8,P15);(A1,I4,M4,E8,P16);(A1,I4,M4,E8,P17);(A1,I4,M4,E8,P18);(A1,I4,M4,E8,P19);(A1,I4,M4,E8,P20);(A1,I4,M4,E9,P1);(A1,I4,M4,E9,P2);(A1,I4,M4,E9,P3);(A1,I4,M4,E9,P4);(A1,I4,M4,E9,P5);(A1,I4,M4,E9,P6);(A1,I4,M4,E9,P7);(A1,I4,M4,E9,P8);(A1,I4,M4,E9,P9);(A1,I4,M4,E9,P10);(A1,I4,M4,E9,P11);(A1,I4,M4,E9,P12);(A1,I4,M4,E9,P13);(A1,I4,M4,E9,P14);(A1,I4,M4,E9,P15);(A1,I4,M4,E9,P16);(A1,I4,M4,E9,P17);(A1,I4,M4,E9,P18);(A1,I4,M4,E9,P19);(A1,I4,M4,E9,P20);(A1,I4,M5,E1,P1);(A1,I4,M5,E1,P2);(A1,I4,M5,E1,P3);(A1,I4,M5,E1,P4);(A1,I4,M5,E1,P5);(A1,I4,M5,E1,P6);(A1,I4,M5,E1,P7);(A1,I4,M5,E1,P8);(A1,I4,M5,E1,P9);(A1,I4,M5,E1,P10);(A1,I4,M5,E1,P11);(A1,I4,M5,E1,P12);(A1,I4,M5,E1,P13);(A1,I4,M5,E1,P14);(A1,I4,M5,E1,P15);(A1,I4,M5,E1,P16);(A1,I4,M5,E1,P17);(A1,I4,M5,E1,P18);(A1,I4,M5,E1,P19);(A1,I4,M5,E1,P20);(A1,I4,M5,E2,P1);(A1,I4,M5,E2,P2);(A1,I4,M5,E2,P3);(A1,I4,M5,E2,P4);(A1,I4,M5,E2,P5);(A1,I4,M5,E2,P6);(A1,I4,M5,E2,P7);(A1,I4,M5,E2,P8);(A1,I4,M5,E2,P9);(A1,I4,M5,E2,P10);(A1,I4,M5,E2,P11);(A1,I4,M5,E2,P12);(A1,I4,M5,E2,P13);(A1,I4,M5,E2,P14);(A1,I4,M5,E2,P15);(A1,I4,M5,E2,P16);(A1,I4,M5,E2,P17);(A1,I4,M5,E2,P18);(A1,I4,M5,E2,P19);(A1,I4,M5,E2,P20);(A1,I4,M5,E3,P1);(A1,I4,M5,E3,P2);(A1,I4,M5,E3,P3);(A1,I4,M5,E3,P4);(A1,I4,M5,E3,P5);(A1,I4,M5,E3,P6);(A1,I4,M5,E3,P7);(A1,I4,M5,E3,P8);(A1,I4,M5,E3,P9);(A1,I4,M5,E3,P10);(A1,I4,M5,E3,P11);(A1,I4,M5,E3,P12);(A1,I4,M5,E3,P13);(A1,I4,M5,E3,P14);(A1,I4,M5,E3,P15);(A1,I4,M5,E3,P16);(A1,I4,M5,E3,P17);(A1,I4,M5,E3,P18);(A1,I4,M5,E3,P19);(A1,I4,M5,E3,P20);(A1,I4,M5,E4,P1);(A1,I4,M5,E4,P2);(A1,I4,M5,E4,P3);(A1,I4,M5,E4,P4);(A1,I4,M5,E4,P5);(A1,I4,M5,E4,P6);(A1,I4,M5,E4,P7);(A1,I4,M5,E4,P8);(A1,I4,M5,E4,P9);(A1,I4,M5,E4,P10);(A1,I4,M5,E4,P11);(A1,I4,M5,E4,P12);(A1,I4,M5,E4,P13);(A1,I4,M5,E4,P14);(A1,I4,M5,E4,P15);(A1,I4,M5,E4,P16);(A1,I4,M5,E4,P17);(A1,I4,M5,E4,P18);(A1,I4,M5,E4,P19);(A1,I4,M5,E4,P20);(A1,I4,M5,E5,P1);(A1,I4,M5,E5,P2);(A1,I4,M5,E5,P3);(A1,I4,M5,E5,P4);(A1,I4,M5,E5,P5);(A1,I4,M5,E5,P6);(A1,I4,M5,E5,P7);(A1,I4,M5,E5,P8);(A1,I4,M5,E5,P9);(A1,I4,M5,E5,P10);(A1,I4,M5,E5,P11);(A1,I4,M5,E5,P12);(A1,I4,M5,E5,P13);(A1,I4,M5,E5,P14);(A1,I4,M5,E5,P15);(A1,I4,M5,E5,P16);(A1,I4,M5,E5,P17);(A1,I4,M5,E5,P18);(A1,I4,M5,E5,P19);(A1,I4,M5,E5,P20);(A1,I4,M5,E6,P1);(A1,I4,M5,E6,P2);(A1,I4,M5,E6,P3);(A1,I4,M5,E6,P4);(A1,I4,M5,E6,P5);(A1,I4,M5,E6,P6);(A1,I4,M5,E6,P7);(A1,I4,M5,E6,P8);(A1,I4,M5,E6,P9);(A1,I4,M5,E6,P10);(A1,I4,M5,E6,P11);(A1,I4,M5,E6,P12);(A1,I4,M5,E6,P13);(A1,I4,M5,E6,P14);(A1,I4,M5,E6,P15);(A1,I4,M5,E6,P16);(A1,I4,M5,E6,P17);(A1,I4,M5,E6,P18);(A1,I4,M5,E6,P19);(A1,I4,M5,E6,P20);(A1,I4,M5,E7,P1);(A1,I4,M5,E7,P2);(A1,I4,M5,E7,P3);(A1,I4,M5,E7,P4);(A1,I4,M5,E7,P5);(A1,I4,M5,E7,P6);(A1,I4,M5,E7,P7);(A1,I4,M5,E7,P8);(A1,I4,M5,E7,P9);(A1,I4,M5,E7,P10);(A1,I4,M5,E7,P11);(A1,I4,M5,E7,P12);(A1,I4,M5,E7,P13);(A1,I4,M5,E7,P14);(A1,I4,M5,E7,P15);(A1,I4,M5,E7,P16);(A1,I4,M5,E7,P17);(A1,I4,M5,E7,P18);(A1,I4,M5,E7,P19);(A1,I4,M5,E7,P20);(A1,I4,M5,E8,P1);(A1,I4,M5,E8,P2);(A1,I4,M5,E8,P3);(A1,I4,M5,E8,P4);(A1,I4,M5,E8,P5);(A1,I4,M5,E8,P6);(A1,I4,M5,E8,P7);(A1,I4,M5,E8,P8);(A1,I4,M5,E8,P9);(A1,I4,M5,E8,P10);(A1,I4,M5,E8,P11);(A1,I4,M5,E8,P12);(A1,I4,M5,E8,P13);(A1,I4,M5,E8,P14);(A1,I4,M5,E8,P15);(A1,I4,M5,E8,P16);(A1,I4,M5,E8,P17);(A1,I4,M5,E8,P18);(A1,I4,M5,E8,P19);(A1,I4,M5,E8,P20);(A1,I4,M5,E9,P1);(A1,I4,M5,E9,P2);(A1,I4,M5,E9,P3);(A1,I4,M5,E9,P4);(A1,I4,M5,E9,P5);(A1,I4,M5,E9,P6);(A1,I4,M5,E9,P7);(A1,I4,M5,E9,P8);(A1,I4,M5,E9,P9);(A1,I4,M5,E9,P10);(A1,I4,M5,E9,P11);(A1,I4,M5,E9,P12);(A1,I4,M5,E9,P13);(A1,I4,M5,E9,P14);(A1,I4,M5,E9,P15);(A1,I4,M5,E9,P16);(A1,I4,M5,E9,P17);(A1,I4,M5,E9,P18);(A1,I4,M5,E9,P19);(A1,I4,M5,E9,P20);(A1,I4,M6,E1,P1);(A1,I4,M6,E1,P2);(A1,I4,M6,E1,P3);(A1,I4,M6,E1,P4);(A1,I4,M6,E1,P5);(A1,I4,M6,E1,P6);(A1,I4,M6,E1,P7);(A1,I4,M6,E1,P8);(A1,I4,M6,E1,P9);(A1,I4,M6,E1,P10);(A1,I4,M6,E1,P11);(A1,I4,M6,E1,P12);(A1,I4,M6,E1,P13);(A1,I4,M6,E1,P14);(A1,I4,M6,E1,P15);(A1,I4,M6,E1,P16);(A1,I4,M6,E1,P17);(A1,I4,M6,E1,P18);(A1,I4,M6,E1,P19);(A1,I4,M6,E1,P20);(A1,I4,M6,E2,P1);(A1,I4,M6,E2,P2);(A1,I4,M6,E2,P3);(A1,I4,M6,E2,P4);(A1,I4,M6,E2,P5);(A1,I4,M6,E2,P6);(A1,I4,M6,E2,P7);(A1,I4,M6,E2,P8);(A1,I4,M6,E2,P9);(A1,I4,M6,E2,P10);(A1,I4,M6,E2,P11);(A1,I4,M6,E2,P12);(A1,I4,M6,E2,P13);(A1,I4,M6,E2,P14);(A1,I4,M6,E2,P15);(A1,I4,M6,E2,P16);(A1,I4,M6,E2,P17);(A1,I4,M6,E2,P18);(A1,I4,M6,E2,P19);(A1,I4,M6,E2,P20);(A1,I4,M6,E3,P1);(A1,I4,M6,E3,P2);(A1,I4,M6,E3,P3);(A1,I4,M6,E3,P4);(A1,I4,M6,E3,P5);(A1,I4,M6,E3,P6);(A1,I4,M6,E3,P7);(A1,I4,M6,E3,P8);(A1,I4,M6,E3,P9);(A1,I4,M6,E3,P10);(A1,I4,M6,E3,P11);(A1,I4,M6,E3,P12);(A1,I4,M6,E3,P13);(A1,I4,M6,E3,P14);(A1,I4,M6,E3,P15);(A1,I4,M6,E3,P16);(A1,I4,M6,E3,P17);(A1,I4,M6,E3,P18);(A1,I4,M6,E3,P19);(A1,I4,M6,E3,P20);(A1,I4,M6,E4,P1);(A1,I4,M6,E4,P2);(A1,I4,M6,E4,P3);(A1,I4,M6,E4,P4);(A1,I4,M6,E4,P5);(A1,I4,M6,E4,P6);(A1,I4,M6,E4,P7);(A1,I4,M6,E4,P8);(A1,I4,M6,E4,P9);(A1,I4,M6,E4,P10);(A1,I4,M6,E4,P11);(A1,I4,M6,E4,P12);(A1,I4,M6,E4,P13);(A1,I4,M6,E4,P14);(A1,I4,M6,E4,P15);(A1,I4,M6,E4,P16);(A1,I4,M6,E4,P17);(A1,I4,M6,E4,P18);(A1,I4,M6,E4,P19);(A1,I4,M6,E4,P20);(A1,I4,M6,E5,P1);(A1,I4,M6,E5,P2);(A1,I4,M6,E5,P3);(A1,I4,M6,E5,P4);(A1,I4,M6,E5,P5);(A1,I4,M6,E5,P6);(A1,I4,M6,E5,P7);(A1,I4,M6,E5,P8);(A1,I4,M6,E5,P9);(A1,I4,M6,E5,P10);(A1,I4,M6,E5,P11);(A1,I4,M6,E5,P12);(A1,I4,M6,E5,P13);(A1,I4,M6,E5,P14);(A1,I4,M6,E5,P15);(A1,I4,M6,E5,P16);(A1,I4,M6,E5,P17);(A1,I4,M6,E5,P18);(A1,I4,M6,E5,P19);(A1,I4,M6,E5,P20);(A1,I4,M6,E6,P1);(A1,I4,M6,E6,P2);(A1,I4,M6,E6,P3);(A1,I4,M6,E6,P4);(A1,I4,M6,E6,P5);(A1,I4,M6,E6,P6);(A1,I4,M6,E6,P7);(A1,I4,M6,E6,P8);(A1,I4,M6,E6,P9);(A1,I4,M6,E6,P10);(A1,I4,M6,E6,P11);(A1,I4,M6,E6,P12);(A1,I4,M6,E6,P13);(A1,I4,M6,E6,P14);(A1,I4,M6,E6,P15);(A1,I4,M6,E6,P16);(A1,I4,M6,E6,P17);(A1,I4,M6,E6,P18);(A1,I4,M6,E6,P19);(A1,I4,M6,E6,P20);(A1,I4,M6,E7,P1);(A1,I4,M6,E7,P2);(A1,I4,M6,E7,P3);(A1,I4,M6,E7,P4);(A1,I4,M6,E7,P5);(A1,I4,M6,E7,P6);(A1,I4,M6,E7,P7);(A1,I4,M6,E7,P8);(A1,I4,M6,E7,P9);(A1,I4,M6,E7,P10);(A1,I4,M6,E7,P11);(A1,I4,M6,E7,P12);(A1,I4,M6,E7,P13);(A1,I4,M6,E7,P14);(A1,I4,M6,E7,P15);(A1,I4,M6,E7,P16);(A1,I4,M6,E7,P17);(A1,I4,M6,E7,P18);(A1,I4,M6,E7,P19);(A1,I4,M6,E7,P20);(A1,I4,M6,E8,P1);(A1,I4,M6,E8,P2);(A1,I4,M6,E8,P3);(A1,I4,M6,E8,P4);(A1,I4,M6,E8,P5);(A1,I4,M6,E8,P6);(A1,I4,M6,E8,P7);(A1,I4,M6,E8,P8);(A1,I4,M6,E8,P9);(A1,I4,M6,E8,P10);(A1,I4,M6,E8,P11);(A1,I4,M6,E8,P12);(A1,I4,M6,E8,P13);(A1,I4,M6,E8,P14);(A1,I4,M6,E8,P15);(A1,I4,M6,E8,P16);(A1,I4,M6,E8,P17);(A1,I4,M6,E8,P18);(A1,I4,M6,E8,P19);(A1,I4,M6,E8,P20);(A1,I4,M6,E9,P1);(A1,I4,M6,E9,P2);(A1,I4,M6,E9,P3);(A1,I4,M6,E9,P4);(A1,I4,M6,E9,P5);(A1,I4,M6,E9,P6);(A1,I4,M6,E9,P7);(A1,I4,M6,E9,P8);(A1,I4,M6,E9,P9);(A1,I4,M6,E9,P10);(A1,I4,M6,E9,P11);(A1,I4,M6,E9,P12);(A1,I4,M6,E9,P13);(A1,I4,M6,E9,P14);(A1,I4,M6,E9,P15);(A1,I4,M6,E9,P16);(A1,I4,M6,E9,P17);(A1,I4,M6,E9,P18);(A1,I4,M6,E9,P19);(A1,I4,M6,E9,P20);(A1,I5,M1,E1,P1);(A1,I5,M1,E1,P2);(A1,I5,M 1,E1,P3);(A1,I5,M 1,E1,P4);(A1,I5,M 1,E1,P5);(A1,I5,M1,E1,P6);(A1,I5,M1,E1,P7);(A1,I5,M1,E1,P8);(A1,I5,M1,E1,P9);(A1,I5,M1,E1,P10);(A1,I5,M1,E1,P11);(A1,I5,M1,E1,P12);(A1,I5,M 1,E1,P13);(A1,I5,M1,E1,P14);(A1,I5,M 1,E1,P15);(A1,I5,M1,E1,P16);(A1,I5,M1,E1,P17);(A1,I5,M1,E1,P18);(A1,I5,M1,E1,P19);(A1,I5,M 1,E1,P20);(A1,I5,M 1,E2,P1);(A1,I5,M 1,E2,P2);(A1,I5,M1,E2,P3);(A1,I5,M1,E2,P4);(A1,I5,M1,E2,P5);(A1,I5,M1,E2,P6);(A1,I5,M1,E2,P7);(A1,I5,M1,E2,P8);(A1,I5,M1,E2,P9);(A1,I5,M1,E2,P10);(A1,I5,M1,E2,P11);(A1,I5,M1,E2,P12);(A1,I5,M1,E2,P13);(A1,I5,M1,E2,P14);(A1,I5,M1,E2,P15);(A1,I5,M1,E2,P16);(A1,I5,M1,E2,P17);(A1,I5,M1,E2,P18);(A1,I5,M1,E2,P19);(A1,I5,M1,E2,P20);(A1,I5,M1,E3,P1);(A1,I5,M1,E3,P2);(A1,I5,M1,E3,P3);(A1,I5,M1,E3,P4);(A1,I5,M1,E3,P5);(A1,I5,M1,E3,P6);(A1,I5,M1,E3,P7);(A1,I5,M1,E3,P8);(A1,I5,M1,E3,P9);(A1,I5,M1,E3,P10);(A1,I5,M1,E3,P11);(A1,I5,M1,E3,P12);(A1,I5,M1,E3,P13);(A1,I5,M1,E3,P14);(A1,I5,M1,E3,P15);(A1,I5,M1,E3,P16);(A1,I5,M1,E3,P17);(A1,I5,M1,E3,P18);(A1,I5,M1,E3,P19);(A1,I5,M1,E3,P20);(A1,I5,M1,E4,P1);(A1,I5,M1,E4,P2);(A1,I5,M1,E4,P3);(A1,I5,M1,E4,P4);(A1,I5,M1,E4,P5);(A1,I5,M1,E4,P6);(A1,I5,M1,E4,P7);(A1,I5,M1,E4,P8);(A1,I5,M1,E4,P9);(A1,I5,M1,E4,P10);(A1,I5,M1,E4,P11);(A1,I5,M1,E4,P12);(A1,I5,M1,E4,P13);(A1,I5,M1,E4,P14);(A1,I5,M1,E4,P15);(A1,I5,M1,E4,P16);(A1,I5,M1,E4,P17);(A1,I5,M1,E4,P18);(A1,I5,M1,E4,P19);(A1,I5,M1,E4,P20);(A1,I5,M1,E5,P1);(A1,I5,M1,E5,P2);(A1,I5,M1,E5,P3);(A1,I5,M1,E5,P4);(A1,I5,M1,E5,P5);(A1,I5,M1,E5,P6);(A1,I5,M1,E5,P7);(A1,I5,M1,E5,P8);(A1,I5,M1,E5,P9);(A1,I5,M1,E5,P10);(A1,I5,M1,E5,P11);(A1,I5,M1,E5,P12);(A1,I5,M1,E5,P13);(A1,I5,M1,E5,P14);(A1,I5,M1,E5,P15);(A1,I5,M1,E5,P16);(A1,I5,M1,E5,P17);(A1,I5,M1,E5,P18);(A1,I5,M1,E5,P19);(A1,I5,M1,E5,P20);(A1,I5,M1,E6,P1);(A1,I5,M1,E6,P2);(A1,I5,M1,E6,P3);(A1,I5,M1,E6,P4);(A1,I5,M1,E6,P5);(A1,I5,M1,E6,P6);(A1,I5,M1,E6,P7);(A1,I5,M1,E6,P8);(A1,I5,M1,E6,P9);(A1,I5,M1,E6,P10);(A1,I5,M1,E6,P11);(A1,I5,M1,E6,P12);(A1,I5,M1,E6,P13);(A1,I5,M1,E6,P14);(A1,I5,M1,E6,P15);(A1,I5,M1,E6,P16);(A1,I5,M1,E6,P17);(A1,I5,M1,E6,P18);(A1,I5,M1,E6,P19);(A1,I5,M1,E6,P20);(A1,I5,M1,E7,P1);(A1,I5,M1,E7,P2);(A1,I5,M1,E7,P3);(A1,I5,M1,E7,P4);(A1,I5,M1,E7,P5);(A1,I5,M1,E7,P6);(A1,I5,M1,E7,P7);(A1,I5,M1,E7,P8);(A1,I5,M1,E7,P9);(A1,I5,M1,E7,P10);(A1,I5,M1,E7,P11);(A1,I5,M1,E7,P12);(A1,I5,M1,E7,P13);(A1,I5,M1,E7,P14);(A1,I5,M1,E7,P15);(A1,I5,M1,E7,P16);(A1,I5,M1,E7,P17);(A1,I5,M1,E7,P18);(A1,I5,M1,E7,P19);(A1,I5,M1,E7,P20);(A1,I5,M1,E8,P1);(A1,I5,M1,E8,P2);(A1,I5,M1,E8,P3);(A1,I5,M1,E8,P4);(A1,I5,M1,E8,P5);(A1,I5,M1,E8,P6);(A1,I5,M1,E8,P7);(A1,I5,M1,E8,P8);(A1,I5,M1,E8,P9);(A1,I5,M1,E8,P10);(A1,I5,M1,E8,P11);(A1,I5,M1,E8,P12);(A1,I5,M1,E8,P13);(A1,I5,M1,E8,P14);(A1,I5,M1,E8,P15);(A1,I5,M1,E8,P16);(A1,I5,M1,E8,P17);(A1,I5,M1,E8,P18);(A1,I5,M1,E8,P19);(A1,I5,M1,E8,P20);(A1,I5,M1,E9,P1);(A1,I5,M1,E9,P2);(A1,I5,M1,E9,P3);(A1,I5,M1,E9,P4);(A1,I5,M1,E9,P5);(A1,I5,M1,E9,P6);(A1,I5,M1,E9,P7);(A1,I5,M1,E9,P8);(A1,I5,M1,E9,P9);(A1,I5,M1,E9,P10);(A1,I5,M1,E9,P11);(A1,I5,M1,E9,P12);(A1,I5,M1,E9,P13);(A1,I5,M1,E9,P14);(A1,I5,M1,E9,P15);(A1,I5,M1,E9,P16);(A1,I5,M1,E9,P17);(A1,I5,M1,E9,P18);(A1,I5,M1,E9,P19);(A1,I5,M1,E9,P20);(A1,I5,M2,E1,P1);(A1,I5,M2,E1,P2);(A1,I5,M2,E1,P3);(A1,I5,M2,E1,P4);(A1,I5,M2,E1,P5);(A1,I5,M2,E1,P6);(A1,I5,M2,E1,P7);(A1,I5,M2,E1,P8);(A1,I5,M2,E1,P9);(A1,I5,M2,E1,P10);(A1,I5,M2,E1,P11);(A1,I5,M2,E1,P12);(A1,I5,M2,E1,P13);(A1,I5,M2,E1,P14);(A1,I5,M2,E1,P15);(A1,I5,M2,E1,P16);(A1,I5,M2,E1,P17);(A1,I5,M2,E1,P18);(A1,I5,M2,E1,P19);(A1,I5,M2,E1,P20);(A1,I5,M2,E2,P1);(A1,I5,M2,E2,P2);(A1,I5,M2,E2,P3);(A1,I5,M2,E2,P4);(A1,I5,M2,E2,P5);(A1,I5,M2,E2,P6);(A1,I5,M2,E2,P7);(A1,I5,M2,E2,P8);(A1,I5,M2,E2,P9);(A1,I5,M2,E2,P10);(A1,I5,M2,E2,P11);(A1,I5,M2,E2,P12);(A1,I5,M2,E2,P13);(A1,I5,M2,E2,P14);(A1,I5,M2,E2,P15);(A1,I5,M2,E2,P16);(A1,I5,M2,E2,P17);(A1,I5,M2,E2,P18);(A1,I5,M2,E2,P19);(A1,I5,M2,E2,P20);(A1,I5,M2,E3,P1);(A1,I5,M2,E3,P2);(A1,I5,M2,E3,P3);(A1,I5,M2,E3,P4);(A1,I5,M2,E3,P5);(A1,I5,M2,E3,P6);(A1,I5,M2,E3,P7);(A1,I5,M2,E3,P8);(A1,I5,M2,E3,P9);(A1,I5,M2,E3,P10);(A1,I5,M2,E3,P11);(A1,I5,M2,E3,P12);(A1,I5,M2,E3,P13);(A1,I5,M2,E3,P14);(A1,I5,M2,E3,P15);(A1,I5,M2,E3,P16);(A1,I5,M2,E3,P17);(A1,I5,M2,E3,P18);(A1,I5,M2,E3,P19);(A1,I5,M2,E3,P20);(A1,I5,M2,E4,P1);(A1,I5,M2,E4,P2);(A1,I5,M2,E4,P3);(A1,I5,M2,E4,P4);(A1,I5,M2,E4,P5);(A1,I5,M2,E4,P6);(A1,I5,M2,E4,P7);(A1,I5,M2,E4,P8);(A1,I5,M2,E4,P9);(A1,I5,M2,E4,P10);(A1,I5,M2,E4,P11);(A1,I5,M2,E4,P12);(A1,I5,M2,E4,P13);(A1,I5,M2,E4,P14);(A1,I5,M2,E4,P15);(A1,I5,M2,E4,P16);(A1,I5,M2,E4,P17);(A1,I5,M2,E4,P18);(A1,I5,M2,E4,P19);(A1,I5,M2,E4,P20);(A1,I5,M2,E5,P1);(A1,I5,M2,E5,P2);(A1,I5,M2,E5,P3);(A1,I5,M2,E5,P4);(A1,I5,M2,E5,P5);(A1,I5,M2,E5,P6);(A1,I5,M2,E5,P7);(A1,I5,M2,E5,P8);(A1,I5,M2,E5,P9);(A1,I5,M2,E5,P10);(A1,I5,M2,E5,P11);(A1,I5,M2,E5,P12);(A1,I5,M2,E5,P13);(A1,I5,M2,E5,P14);(A1,I5,M2,E5,P15);(A1,I5,M2,E5,P16);(A1,I5,M2,E5,P17);(A1,I5,M2,E5,P18);(A1,I5,M2,E5,P19);(A1,I5,M2,E5,P20);(A1,I5,M2,E6,P1);(A1,I5,M2,E6,P2);(A1,I5,M2,E6,P3);(A1,I5,M2,E6,P4);(A1,I5,M2,E6,P5);(A1,I5,M2,E6,P6);(A1,I5,M2,E6,P7);(A1,I5,M2,E6,P8);(A1,I5,M2,E6,P9);(A1,I5,M2,E6,P10);(A1,I5,M2,E6,P11);(A1,I5,M2,E6,P12);(A1,I5,M2,E6,P13);(A1,I5,M2,E6,P14);(A1,I5,M2,E6,P15);(A1,I5,M2,E6,P16);(A1,I5,M2,E6,P17);(A1,I5,M2,E6,P18);(A1,I5,M2,E6,P19);(A1,I5,M2,E6,P20);(A1,I5,M2,E7,P1);(A1,I5,M2,E7,P2);(A1,I5,M2,E7,P3);(A1,I5,M2,E7,P4);(A1,I5,M2,E7,P5);(A1,I5,M2,E7,P6);(A1,I5,M2,E7,P7);(A1,I5,M2,E7,P8);(A1,I5,M2,E7,P9);(A1,I5,M2,E7,P10);(A1,I5,M2,E7,P11);(A1,I5,M2,E7,P12);(A1,I5,M2,E7,P13);(A1,I5,M2,E7,P14);(A1,I5,M2,E7,P15);(A1,I5,M2,E7,P16);(A1,I5,M2,E7,P17);(A1,I5,M2,E7,P18);(A1,I5,M2,E7,P19);(A1,I5,M2,E7,P20);(A1,I5,M2,E8,P1);(A1,I5,M2,E8,P2);(A1,I5,M2,E8,P3);(A1,I5,M2,E8,P4);(A1,I5,M2,E8,P5);(A1,I5,M2,E8,P6);(A1,I5,M2,E8,P7);(A1,I5,M2,E8,P8);(A1,I5,M2,E8,P9);(A1,I5,M2,E8,P10);(A1,I5,M2,E8,P11);(A1,I5,M2,E8,P12);(A1,I5,M2,E8,P13);(A1,I5,M2,E8,P14);(A1,I5,M2,E8,P15);(A1,I5,M2,E8,P16);(A1,I5,M2,E8,P17);(A1,I5,M2,E8,P18);(A1,I5,M2,E8,P19);(A1,I5,M2,E8,P20);(A1,I5,M2,E9,P1);(A1,I5,M2,E9,P2);(A1,I5,M2,E9,P3);(A1,I5,M2,E9,P4);(A1,I5,M2,E9,P5);(A1,I5,M2,E9,P6);(A1,I5,M2,E9,P7);(A1,I5,M2,E9,P8);(A1,I5,M2,E9,P9);(A1,I5,M2,E9,P10);(A1,I5,M2,E9,P11);(A1,I5,M2,E9,P12);(A1,I5,M2,E9,P13);(A1,I5,M2,E9,P14);(A1,I5,M2,E9,P15);(A1,I5,M2,E9,P16);(A1,I5,M2,E9,P17);(A1,I5,M2,E9,P18);(A1,I5,M2,E9,P19);(A1,I5,M2,E9,P20);(A1,I5,M3,E1,P1);(A1,I5,M3,E1,P2);(A1,I5,M3,E1,P3);(A1,I5,M3,E1,P4);(A1,I5,M3,E1,P5);(A1,I5,M3,E1,P6);(A1,I5,M3,E1,P7);(A1,I5,M3,E1,P8);(A1,I5,M3,E1,P9);(A1,I5,M3,E1,P10);(A1,I5,M3,E1,P11);(A1,I5,M3,E1,P12);(A1,I5,M3,E1,P13);(A1,I5,M3,E1,P14);(A1,I5,M3,E1,P15);(A1,I5,M3,E1,P16);(A1,I5,M3,E1,P17);(A1,I5,M3,E1,P18);(A1,I5,M3,E1,P19);(A1,I5,M3,E1,P20);(A1,I5,M3,E2,P1);(A1,I5,M3,E2,P2);(A1,I5,M3,E2,P3);(A1,I5,M3,E2,P4);(A1,I5,M3,E2,P5);(A1,I5,M3,E2,P6);(A1,I5,M3,E2,P7);(A1,I5,M3,E2,P8);(A1,I5,M3,E2,P9);(A1,I5,M3,E2,P10);(A1,I5,M3,E2,P11);(A1,I5,M3,E2,P12);(A1,I5,M3,E2,P13);(A1,I5,M3,E2,P14);(A1,I5,M3,E2,P15);(A1,I5,M3,E2,P16);(A1,I5,M3,E2,P17);(A1,I5,M3,E2,P18);(A1,I5,M3,E2,P19);(A1,I5,M3,E2,P20);(A1,I5,M3,E3,P1);(A1,I5,M3,E3,P2);(A1,I5,M3,E3,P3);(A1,I5,M3,E3,P4);(A1,I5,M3,E3,P5);(A1,I5,M3,E3,P6);(A1,I5,M3,E3,P7);(A1,I5,M3,E3,P8);(A1,I5,M3,E3,P9);(A1,I5,M3,E3,P10);(A1,I5,M3,E3,P11);(A1,I5,M3,E3,P12);(A1,I5,M3,E3,P13);(A1,I5,M3,E3,P14);(A1,I5,M3,E3,P15);(A1,I5,M3,E3,P16);(A1,I5,M3,E3,P17);(A1,I5,M3,E3,P18);(A1,I5,M3,E3,P19);(A1,I5,M3,E3,P20);(A1,I5,M3,E4,P1);(A1,I5,M3,E4,P2);(A1,I5,M3,E4,P3);(A1,I5,M3,E4,P4);(A1,I5,M3,E4,P5);(A1,I5,M3,E4,P6);(A1,I5,M3,E4,P7);(A1,I5,M3,E4,P8);(A1,I5,M3,E4,P9);(A1,I5,M3,E4,P10);(A1,I5,M3,E4,P11);(A1,I5,M3,E4,P12);(A1,I5,M3,E4,P13);(A1,I5,M3,E4,P14);(A1,I5,M3,E4,P15);(A1,I5,M3,E4,P16);(A1,I5,M3,E4,P17);(A1,I5,M3,E4,P18);(A1,I5,M3,E4,P19);(A1,I5,M3,E4,P20);(A1,I5,M3,E5,P1);(A1,I5,M3,E5,P2);(A1,I5,M3,E5,P3);(A1,I5,M3,E5,P4);(A1,I5,M3,E5,P5);(A1,I5,M3,E5,P6);(A1,I5,M3,E5,P7);(A1,I5,M3,E5,P8);(A1,I5,M3,E5,P9);(A1,I5,M3,E5,P10);(A1,I5,M3,E5,P11);(A1,I5,M3,E5,P12);(A1,I5,M3,E5,P13);(A1,I5,M3,E5,P14);(A1,I5,M3,E5,P15);(A1,I5,M3,E5,P16);(A1,I5,M3,E5,P17);(A1,I5,M3,E5,P18);(A1,I5,M3,E5,P19);(A1,I5,M3,E5,P20);(A1,I5,M3,E6,P1);(A1,I5,M3,E6,P2);(A1,I5,M3,E6,P3);(A1,I5,M3,E6,P4);(A1,I5,M3,E6,P5);(A1,I5,M3,E6,P6);(A1,I5,M3,E6,P7);(A1,I5,M3,E6,P8);(A1,I5,M3,E6,P9);(A1,I5,M3,E6,P10);(A1,I5,M3,E6,P11);(A1,I5,M3,E6,P12);(A1,I5,M3,E6,P13);(A1,I5,M3,E6,P14);(A1,I5,M3,E6,P15);(A1,I5,M3,E6,P16);(A1,I5,M3,E6,P17);(A1,I5,M3,E6,P18);(A1,I5,M3,E6,P19);(A1,I5,M3,E6,P20);(A1,I5,M3,E7,P1);(A1,I5,M3,E7,P2);(A1,I5,M3,E7,P3);(A1,I5,M3,E7,P4);(A1,I5,M3,E7,P5);(A1,I5,M3,E7,P6);(A1,I5,M3,E7,P7);(A1,I5,M3,E7,P8);(A1,I5,M3,E7,P9);(A1,I5,M3,E7,P10);(A1,I5,M3,E7,P11);(A1,I5,M3,E7,P12);(A1,I5,M3,E7,P13);(A1,I5,M3,E7,P14);(A1,I5,M3,E7,P15);(A1,I5,M3,E7,P16);(A1,I5,M3,E7,P17);(A1,I5,M3,E7,P18);(A1,I5,M3,E7,P19);(A1,I5,M3,E7,P20);(A1,I5,M3,E8,P1);(A1,I5,M3,E8,P2);(A1,I5,M3,E8,P3);(A1,I5,M3,E8,P4);(A1,I5,M3,E8,P5);(A1,I5,M3,E8,P6);(A1,I5,M3,E8,P7);(A1,I5,M3,E8,P8);(A1,I5,M3,E8,P9);(A1,I5,M3,E8,P10);(A1,I5,M3,E8,P11);(A1,I5,M3,E8,P12);(A1,I5,M3,E8,P13);(A1,I5,M3,E8,P14);(A1,I5,M3,E8,P15);(A1,I5,M3,E8,P16);(A1,I5,M3,E8,P17);(A1,I5,M3,E8,P18);(A1,I5,M3,E8,P19);(A1,I5,M3,E8,P20);(A1,I5,M3,E9,P1);(A1,I5,M3,E9,P2);(A1,I5,M3,E9,P3);(A1,I5,M3,E9,P4);(A1,I5,M3,E9,P5);(A1,I5,M3,E9,P6);(A1,I5,M3,E9,P7);(A1,I5,M3,E9,P8);(A1,I5,M3,E9,P9);(A1,I5,M3,E9,P10);(A1,I5,M3,E9,P11);(A1,I5,M3,E9,P12);(A1,I5,M3,E9,P13);(A1,I5,M3,E9,P14);(A1,I5,M3,E9,P15);(A1,I5,M3,E9,P16);(A1,I5,M3,E9,P17);(A1,I5,M3,E9,P18);(A1,I5,M3,E9,P19);(A1,I5,M3,E9,P20);(A1,I5,M4,E1,P1);(A1,I5,M4,E1,P2);(A1,I5,M4,E1,P3);(A1,I5,M4,E1,P4);(A1,I5,M4,E1,P5);(A1,I5,M4,E1,P6);(A1,I5,M4,E1,P7);(A1,I5,M4,E1,P8);(A1,I5,M4,E1,P9);(A1,I5,M4,E1,P10);(A1,I5,M4,E1,P11);(A1,I5,M4,E1,P12);(A1,I5,M4,E1,P13);(A1,I5,M4,E1,P14);(A1,I5,M4,E1,P15);(A1,I5,M4,E1,P16);(A1,I5,M4,E1,P17);(A1,I5,M4,E1,P18);(A1,I5,M4,E1,P19);(A1,I5,M4,E1,P20);(A1,I5,M4,E2,P1);(A1,I5,M4,E2,P2);(A1,I5,M4,E2,P3);(A1,I5,M4,E2,P4);(A1,I5,M4,E2,P5);(A1,I5,M4,E2,P6);(A1,I5,M4,E2,P7);(A1,I5,M4,E2,P8);(A1,I5,M4,E2,P9);(A1,I5,M4,E2,P10);(A1,I5,M4,E2,P11);(A1,I5,M4,E2,P12);(A1,I5,M4,E2,P13);(A1,I5,M4,E2,P14);(A1,I5,M4,E2,P15);(A1,I5,M4,E2,P16);(A1,I5,M4,E2,P17);(A1,I5,M4,E2,P18);(A1,I5,M4,E2,P19);(A1,I5,M4,E2,P20);(A1,I5,M4,E3,P1);(A1,I5,M4,E3,P2);(A1,I5,M4,E3,P3);(A1,I5,M4,E3,P4);(A1,I5,M4,E3,P5);(A1,I5,M4,E3,P6);(A1,I5,M4,E3,P7);(A1,I5,M4,E3,P8);(A1,I5,M4,E3,P9);(A1,I5,M4,E3,P10);(A1,I5,M4,E3,P11);(A1,I5,M4,E3,P12);(A1,I5,M4,E3,P13);(A1,I5,M4,E3,P14);(A1,I5,M4,E3,P15);(A1,I5,M4,E3,P16);(A1,I5,M4,E3,P17);(A1,I5,M4,E3,P18);(A1,I5,M4,E3,P19);(A1,I5,M4,E3,P20);(A1,I5,M4,E4,P1);(A1,I5,M4,E4,P2);(A1,I5,M4,E4,P3);(A1,I5,M4,E4,P4);(A1,I5,M4,E4,P5);(A1,I5,M4,E4,P6);(A1,I5,M4,E4,P7);(A1,I5,M4,E4,P8);(A1,I5,M4,E4,P9);(A1,I5,M4,E4,P10);(A1,I5,M4,E4,P11);(A1,I5,M4,E4,P12);(A1,I5,M4,E4,P13);(A1,I5,M4,E4,P14);(A1,I5,M4,E4,P15);(A1,I5,M4,E4,P16);(A1,I5,M4,E4,P17);(A1,I5,M4,E4,P18);(A1,I5,M4,E4,P19);(A1,I5,M4,E4,P20);(A1,I5,M4,E5,P1);(A1,I5,M4,E5,P2);(A1,I5,M4,E5,P3);(A1,I5,M4,E5,P4);(A1,I5,M4,E5,P5);(A1,I5,M4,E5,P6);(A1,I5,M4,E5,P7);(A1,I5,M4,E5,P8);(A1,I5,M4,E5,P9);(A1,I5,M4,E5,P10);(A1,I5,M4,E5,P11);(A1,I5,M4,E5,P12);(A1,I5,M4,E5,P13);(A1,I5,M4,E5,P14);(A1,I5,M4,E5,P15);(A1,I5,M4,E5,P16);(A1,I5,M4,E5,P17);(A1,I5,M4,E5,P18);(A1,I5,M4,E5,P19);(A1,I5,M4,E5,P20);(A1,I5,M4,E6,P1);(A1,I5,M4,E6,P2);(A1,I5,M4,E6,P3);(A1,I5,M4,E6,P4);(A1,I5,M4,E6,P5);(A1,I5,M4,E6,P6);(A1,I5,M4,E6,P7);(A1,I5,M4,E6,P8);(A1,I5,M4,E6,P9);(A1,I5,M4,E6,P10);(A1,I5,M4,E6,P11);(A1,I5,M4,E6,P12);(A1,I5,M4,E6,P13);(A1,I5,M4,E6,P14);(A1,I5,M4,E6,P15);(A1,I5,M4,E6,P16);(A1,I5,M4,E6,P17);(A1,I5,M4,E6,P18);(A1,I5,M4,E6,P19);(A1,I5,M4,E6,P20);(A1,I5,M4,E7,P1);(A1,I5,M4,E7,P2);(A1,I5,M4,E7,P3);(A1,I5,M4,E7,P4);(A1,I5,M4,E7,P5);(A1,I5,M4,E7,P6);(A1,I5,M4,E7,P7);(A1,I5,M4,E7,P8);(A1,I5,M4,E7,P9);(A1,I5,M4,E7,P10);(A1,I5,M4,E7,P11);(A1,I5,M4,E7,P12);(A1,I5,M4,E7,P13);(A1,I5,M4,E7,P14);(A1,I5,M4,E7,P15);(A1,I5,M4,E7,P16);(A1,I5,M4,E7,P17);(A1,I5,M4,E7,P18);(A1,I5,M4,E7,P19);(A1,I5,M4,E7,P20);(A1,I5,M4,E8,P1);(A1,I5,M4,E8,P2);(A1,I5,M4,E8,P3);(A1,I5,M4,E8,P4);(A1,I5,M4,E8,P5);(A1,I5,M4,E8,P6);(A1,I5,M4,E8,P7);(A1,I5,M4,E8,P8);(A1,I5,M4,E8,P9);(A1,I5,M4,E8,P10);(A1,I5,M4,E8,P11);(A1,I5,M4,E8,P12);(A1,I5,M4,E8,P13);(A1,I5,M4,E8,P14);(A1,I5,M4,E8,P15);(A1,I5,M4,E8,P16);(A1,I5,M4,E8,P17);(A1,I5,M4,E8,P18);(A1,I5,M4,E8,P19);(A1,I5,M4,E8,P20);(A1,I5,M4,E9,P1);(A1,I5,M4,E9,P2);(A1,I5,M4,E9,P3);(A1,I5,M4,E9,P4);(A1,I5,M4,E9,P5);(A1,I5,M4,E9,P6);(A1,I5,M4,E9,P7);(A1,I5,M4,E9,P8);(A1,I5,M4,E9,P9);(A1,I5,M4,E9,P10);(A1,I5,M4,E9,P11);(A1,I5,M4,E9,P12);(A1,I5,M4,E9,P13);(A1,I5,M4,E9,P14);(A1,I5,M4,E9,P15);(A1,I5,M4,E9,P16);(A1,I5,M4,E9,P17);(A1,I5,M4,E9,P18);(A1,I5,M4,E9,P19);(A1,I5,M4,E9,P20);(A1,I5,M5,E1,P1);(A1,I5,M5,E1,P2);(A1,I5,M5,E1,P3);(A1,I5,M5,E1,P4);(A1,I5,M5,E1,P5);(A1,I5,M5,E1,P6);(A1,I5,M5,E1,P7);(A1,I5,M5,E1,P8);(A1,I5,M5,E1,P9);(A1,I5,M5,E1,P10);(A1,I5,M5,E1,P11);(A1,I5,M5,E1,P12);(A1,I5,M5,E1,P13);(A1,I5,M5,E1,P14);(A1,I5,M5,E1,P15);(A1,I5,M5,E1,P16);(A1,I5,M5,E1,P17);(A1,I5,M5,E1,P18);(A1,I5,M5,E1,P19);(A1,I5,M5,E1,P20);(A1,I5,M5,E2,P1);(A1,I5,M5,E2,P2);(A1,I5,M5,E2,P3);(A1,I5,M5,E2,P4);(A1,I5,M5,E2,P5);(A1,I5,M5,E2,P6);(A1,I5,M5,E2,P7);(A1,I5,M5,E2,P8);(A1,I5,M5,E2,P9);(A1,I5,M5,E2,P10);(A1,I5,M5,E2,P11);(A1,I5,M5,E2,P12);(A1,I5,M5,E2,P13);(A1,I5,M5,E2,P14);(A1,I5,M5,E2,P15);(A1,I5,M5,E2,P16);(A1,I5,M5,E2,P17);(A1,I5,M5,E2,P18);(A1,I5,M5,E2,P19);(A1,I5,M5,E2,P20);(A1,I5,M5,E3,P1);(A1,I5,M5,E3,P2);(A1,I5,M5,E3,P3);(A1,I5,M5,E3,P4);(A1,I5,M5,E3,P5);(A1,I5,M5,E3,P6);(A1,I5,M5,E3,P7);(A1,I5,M5,E3,P8);(A1,I5,M5,E3,P9);(A1,I5,M5,E3,P10);(A1,I5,M5,E3,P11);(A1,I5,M5,E3,P12);(A1,I5,M5,E3,P13);(A1,I5,M5,E3,P14);(A1,I5,M5,E3,P15);(A1,I5,M5,E3,P16);(A1,I5,M5,E3,P17);(A1,I5,M5,E3,P18);(A1,I5,M5,E3,P19);(A1,I5,M5,E3,P20);(A1,I5,M5,E4,P1);(A1,I5,M5,E4,P2);(A1,I5,M5,E4,P3);(A1,I5,M5,E4,P4);(A1,I5,M5,E4,P5);(A1,I5,M5,E4,P6);(A1,I5,M5,E4,P7);(A1,I5,M5,E4,P8);(A1,I5,M5,E4,P9);(A1,I5,M5,E4,P10);(A1,I5,M5,E4,P11);(A1,I5,M5,E4,P12);(A1,I5,M5,E4,P13);(A1,I5,M5,E4,P14);(A1,I5,M5,E4,P15);(A1,I5,M5,E4,P16);(A1,I5,M5,E4,P17);(A1,I5,M5,E4,P18);(A1,I5,M5,E4,P19);(A1,I5,M5,E4,P20);(A1,I5,M5,E5,P1);(A1,I5,M5,E5,P2);(A1,I5,M5,E5,P3);(A1,I5,M5,E5,P4);(A1,I5,M5,E5,P5);(A1,I5,M5,E5,P6);(A1,I5,M5,E5,P7);(A1,I5,M5,E5,P8);(A1,I5,M5,E5,P9);(A1,I5,M5,E5,P10);(A1,I5,M5,E5,P11);(A1,I5,M5,E5,P12);(A1,I5,M5,E5,P13);(A1,I5,M5,E5,P14);(A1,I5,M5,E5,P15);(A1,I5,M5,E5,P16);(A1,I5,M5,E5,P17);(A1,I5,M5,E5,P18);(A1,I5,M5,E5,P19);(A1,I5,M5,E5,P20);(A1,I5,M5,E6,P1);(A1,I5,M5,E6,P2);(A1,I5,M5,E6,P3);(A1,I5,M5,E6,P4);(A1,I5,M5,E6,P5);(A1,I5,M5,E6,P6);(A1,I5,M5,E6,P7);(A1,I5,M5,E6,P8);(A1,I5,M5,E6,P9);(A1,I5,M5,E6,P10);(A1,I5,M5,E6,P11);(A1,I5,M5,E6,P12);(A1,I5,M5,E6,P13);(A1,I5,M5,E6,P14);(A1,I5,M5,E6,P15);(A1,I5,M5,E6,P16);(A1,I5,M5,E6,P17);(A1,I5,M5,E6,P18);(A1,I5,M5,E6,P19);(A1,I5,M5,E6,P20);(A1,I5,M5,E7,P1);(A1,I5,M5,E7,P2);(A1,I5,M5,E7,P3);(A1,I5,M5,E7,P4);(A1,I5,M5,E7,P5);(A1,I5,M5,E7,P6);(A1,I5,M5,E7,P7);(A1,I5,M5,E7,P8);(A1,I5,M5,E7,P9);(A1,I5,M5,E7,P10);(A1,I5,M5,E7,P11);(A1,I5,M5,E7,P12);(A1,I5,M5,E7,P13);(A1,I5,M5,E7,P14);(A1,I5,M5,E7,P15);(A1,I5,M5,E7,P16);(A1,I5,M5,E7,P17);(A1,I5,M5,E7,P18);(A1,I5,M5,E7,P19);(A1,I5,M5,E7,P20);(A1,I5,M5,E8,P1);(A1,I5,M5,E8,P2);(A1,I5,M5,E8,P3);(A1,I5,M5,E8,P4);(A1,I5,M5,E8,P5);(A1,I5,M5,E8,P6);(A1,I5,M5,E8,P7);(A1,I5,M5,E8,P8);(A1,I5,M5,E8,P9);(A1,I5,M5,E8,P10);(A1,I5,M5,E8,P11);(A1,I5,M5,E8,P12);(A1,I5,M5,E8,P13);(A1,I5,M5,E8,P14);(A1,I5,M5,E8,P15);(A1,I5,M5,E8,P16);(A1,I5,M5,E8,P17);(A1,I5,M5,E8,P18);(A1,I5,M5,E8,P19);(A1,I5,M5,E8,P20);(A1,I5,M5,E9,P1);(A1,I5,M5,E9,P2);(A1,I5,M5,E9,P3);(A1,I5,M5,E9,P4);(A1,I5,M5,E9,P5);(A1,I5,M5,E9,P6);(A1,I5,M5,E9,P7);(A1,I5,M5,E9,P8);(A1,I5,M5,E9,P9);(A1,I5,M5,E9,P10);(A1,I5,M5,E9,P11);(A1,I5,M5,E9,P12);(A1,I5,M5,E9,P13);(A1,I5,M5,E9,P14);(A1,I5,M5,E9,P15);(A1,I5,M5,E9,P16);(A1,I5,M5,E9,P17);(A1,I5,M5,E9,P18);(A1,I5,M5,E9,P19);(A1,I5,M5,E9,P20);(A1,I5,M6,E1,P1);(A1,I5,M6,E1,P2);(A1,I5,M6,E1,P3);(A1,I5,M6,E1,P4);(A1,I5,M6,E1,P5);(A1,I5,M6,E1,P6);(A1,I5,M6,E1,P7);(A1,I5,M6,E1,P8);(A1,I5,M6,E1,P9);(A1,I5,M6,E1,P10);(A1,I5,M6,E1,P11);(A1,I5,M6,E1,P12);(A1,I5,M6,E1,P13);(A1,I5,M6,E1,P14);(A1,I5,M6,E1,P15);(A1,I5,M6,E1,P16);(A1,I5,M6,E1,P17);(A1,I5,M6,E1,P18);(A1,I5,M6,E1,P19);(A1,I5,M6,E1,P20);(A1,I5,M6,E2,P1);(A1,I5,M6,E2,P2);(A1,I5,M6,E2,P3);(A1,I5,M6,E2,P4);(A1,I5,M6,E2,P5);(A1,I5,M6,E2,P6);(A1,I5,M6,E2,P7);(A1,I5,M6,E2,P8);(A1,I5,M6,E2,P9);(A1,I5,M6,E2,P10);(A1,I5,M6,E2,P11);(A1,I5,M6,E2,P12);(A1,I5,M6,E2,P13);(A1,I5,M6,E2,P14);(A1,I5,M6,E2,P15);(A1,I5,M6,E2,P16);(A1,I5,M6,E2,P17);(A1,I5,M6,E2,P18);(A1,I5,M6,E2,P19);(A1,I5,M6,E2,P20);(A1,I5,M6,E3,P1);(A1,I5,M6,E3,P2);(A1,I5,M6,E3,P3);(A1,I5,M6,E3,P4);(A1,I5,M6,E3,P5);(A1,I5,M6,E3,P6);(A1,I5,M6,E3,P7);(A1,I5,M6,E3,P8);(A1,I5,M6,E3,P9);(A1,I5,M6,E3,P10);(A1,I5,M6,E3,P11);(A1,I5,M6,E3,P12);(A1,I5,M6,E3,P13);(A1,I5,M6,E3,P14);(A1,I5,M6,E3,P15);(A1,I5,M6,E3,P16);(A1,I5,M6,E3,P17);(A1,I5,M6,E3,P18);(A1,I5,M6,E3,P19);(A1,I5,M6,E3,P20);(A1,I5,M6,E4,P1);(A1,I5,M6,E4,P2);(A1,I5,M6,E4,P3);(A1,I5,M6,E4,P4);(A1,I5,M6,E4,P5);(A1,I5,M6,E4,P6);(A1,I5,M6,E4,P7);(A1,I5,M6,E4,P8);(A1,I5,M6,E4,P9);(A1,I5,M6,E4,P10);(A1,I5,M6,E4,P11);(A1,I5,M6,E4,P12);(A1,I5,M6,E4,P13);(A1,I5,M6,E4,P14);(A1,I5,M6,E4,P15);(A1,I5,M6,E4,P16);(A1,I5,M6,E4,P17);(A1,I5,M6,E4,P18);(A1,I5,M6,E4,P19);(A1,I5,M6,E4,P20);(A1,I5,M6,E5,P1);(A1,I5,M6,E5,P2);(A1,I5,M6,E5,P3);(A1,I5,M6,E5,P4);(A1,I5,M6,E5,P5);(A1,I5,M6,E5,P6);(A1,I5,M6,E5,P7);(A1,I5,M6,E5,P8);(A1,I5,M6,E5,P9);(A1,I5,M6,E5,P10);(A1,I5,M6,E5,P11);(A1,I5,M6,E5,P12);(A1,I5,M6,E5,P13);(A1,I5,M6,E5,P14);(A1,I5,M6,E5,P15);(A1,I5,M6,E5,P16);(A1,I5,M6,E5,P17);(A1,I5,M6,E5,P18);(A1,I5,M6,E5,P19);(A1,I5,M6,E5,P20);(A1,I5,M6,E6,P1);(A1,I5,M6,E6,P2);(A1,I5,M6,E6,P3);(A1,I5,M6,E6,P4);(A1,I5,M6,E6,P5);(A1,I5,M6,E6,P6);(A1,I5,M6,E6,P7);(A1,I5,M6,E6,P8);(A1,I5,M6,E6,P9);(A1,I5,M6,E6,P10);(A1,I5,M6,E6,P11);(A1,I5,M6,E6,P12);(A1,I5,M6,E6,P13);(A1,I5,M6,E6,P14);(A1,I5,M6,E6,P15);(A1,I5,M6,E6,P16);(A1,I5,M6,E6,P17);(A1,I5,M6,E6,P18);(A1,I5,M6,E6,P19);(A1,I5,M6,E6,P20);(A1,I5,M6,E7,P1);(A1,I5,M6,E7,P2);(A1,I5,M6,E7,P3);(A1,I5,M6,E7,P4);(A1,I5,M6,E7,P5);(A1,I5,M6,E7,P6);(A1,I5,M6,E7,P7);(A1,I5,M6,E7,P8);(A1,I5,M6,E7,P9);(A1,I5,M6,E7,P10);(A1,I5,M6,E7,P11);(A1,I5,M6,E7,P12);(A1,I5,M6,E7,P13);(A1,I5,M6,E7,P14);(A1,I5,M6,E7,P15);(A1,I5,M6,E7,P16);(A1,I5,M6,E7,P17);(A1,I5,M6,E7,P18);(A1,I5,M6,E7,P19);(A1,I5,M6,E7,P20);(A1,I5,M6,E8,P1);(A1,I5,M6,E8,P2);(A1,I5,M6,E8,P3);(A1,I5,M6,E8,P4);(A1,I5,M6,E8,P5);(A1,I5,M6,E8,P6);(A1,I5,M6,E8,P7);(A1,I5,M6,E8,P8);(A1,I5,M6,E8,P9);(A1,I5,M6,E8,P10);(A1,I5,M6,E8,P11);(A1,I5,M6,E8,P12);(A1,I5,M6,E8,P13);(A1,I5,M6,E8,P14);(A1,I5,M6,E8,P15);(A1,I5,M6,E8,P16);(A1,I5,M6,E8,P17);(A1,I5,M6,E8,P18);(A1,I5,M6,E8,P19);(A1,I5,M6,E8,P20);(A1,I5,M6,E9,P1);(A1,I5,M6,E9,P2);(A1,I5,M6,E9,P3);(A1,I5,M6,E9,P4);(A1,I5,M6,E9,P5);(A1,I5,M6,E9,P6);(A1,I5,M6,E9,P7);(A1,I5,M6,E9,P8);(A1,I5,M6,E9,P9);(A1,I5,M6,E9,P10);(A1,I5,M6,E9,P11);(A1,I5,M6,E9,P12);(A1,I5,M6,E9,P13);(A1,I5,M6,E9,P14);(A1,I5,M6,E9,P15);(A1,I5,M6,E9,P16);(A1,I5,M6,E9,P17);(A1,I5,M6,E9,P18);(A1,I5,M6,E9,P19);(A1,I5,M6,E9,P20);(A1,I6,M1,E1,P1);(A1,I6,M1,E1,P2);(A1,I6,M1,E1,P3);(A1,I6,M1,E1,P4);(A1,I6,M1,E1,P5);(A1,I6,M1,E1,P6);(A1,I6,M1,E1,P7);(A1,I6,M1,E1,P8);(A1,I6,M1,E1,P9);(A1,I6,M1,E1,P10);(A1,I6,M1,E1,P11);(A1,I6,M1,E1,P12);(A1,I6,M1,E1,P13);(A1,I6,M1,E1,P14);(A1,I6,M1,E1,P15);(A1,I6,M1,E1,P16);(A1,I6,M1,E1,P17);(A1,I6,M1,E1,P18);(A1,I6,M1,E1,P19);(A1,I6,M1,E1,P20);(A1,I6,M1,E2,P1);(A1,I6,M1,E2,P2);(A1,I6,M1,E2,P3);(A1,I6,M1,E2,P4);(A1,I6,M1,E2,P5);(A1,I6,M1,E2,P6);(A1,I6,M1,E2,P7);(A1,I6,M1,E2,P8);(A1,I6,M1,E2,P9);(A1,I6,M1,E2,P10);(A1,I6,M1,E2,P11);(A1,I6,M1,E2,P12);(A1,I6,M1,E2,P13);(A1,I6,M1,E2,P14);(A1,I6,M1,E2,P15);(A1,I6,M1,E2,P16);(A1,I6,M1,E2,P17);(A1,I6,M1,E2,P18);(A1,I6,M1,E2,P19);(A1,I6,M1,E2,P20);(A1,I6,M1,E3,P1);(A1,I6,M1,E3,P2);(A1,I6,M1,E3,P3);(A1,I6,M1,E3,P4);(A1,I6,M1,E3,P5);(A1,I6,M1,E3,P6);(A1,I6,M1,E3,P7);(A1,I6,M1,E3,P8);(A1,I6,M1,E3,P9);(A1,I6,M1,E3,P10);(A1,I6,M1,E3,P11);(A1,I6,M1,E3,P12);(A1,I6,M1,E3,P13);(A1,I6,M1,E3,P14);(A1,I6,M1,E3,P15);(A1,I6,M1,E3,P16);(A1,I6,M1,E3,P17);(A1,I6,M1,E3,P18);(A1,I6,M1,E3,P19);(A1,I6,M1,E3,P20);(A1,I6,M1,E4,P1);(A1,I6,M1,E4,P2);(A1,I6,M1,E4,P3);(A1,I6,M1,E4,P4);(A1,I6,M1,E4,P5);(A1,I6,M1,E4,P6);(A1,I6,M1,E4,P7);(A1,I6,M1,E4,P8);(A1,I6,M1,E4,P9);(A1,I6,M1,E4,P10);(A1,I6,M1,E4,P11);(A1,I6,M1,E4,P12);(A1,I6,M1,E4,P13);(A1,I6,M1,E4,P14);(A1,I6,M1,E4,P15);(A1,I6,M1,E4,P16);(A1,I6,M1,E4,P17);(A1,I6,M1,E4,P18);(A1,I6,M1,E4,P19);(A1,I6,M1,E4,P20);(A1,I6,M1,E5,P1);(A1,I6,M1,E5,P2);(A1,I6,M1,E5,P3);(A1,I6,M1,E5,P4);(A1,I6,M1,E5,P5);(A1,I6,M1,E5,P6);(A1,I6,M1,E5,P7);(A1,I6,M1,E5,P8);(A1,I6,M1,E5,P9);(A1,I6,M1,E5,P10);(A1,I6,M1,E5,P11);(A1,I6,M1,E5,P12);(A1,I6,M1,E5,P13);(A1,I6,M1,E5,P14);(A1,I6,M1,E5,P15);(A1,I6,M1,E5,P16);(A1,I6,M1,E5,P17);(A1,I6,M1,E5,P18);(A1,I6,M1,E5,P19);(A1,I6,M1,E5,P20);(A1,I6,M1,E6,P1);(A1,I6,M1,E6,P2);(A1,I6,M1,E6,P3);(A1,I6,M1,E6,P4);(A1,I6,M1,E6,P5);(A1,I6,M1,E6,P6);(A1,I6,M1,E6,P7);(A1,I6,M1,E6,P8);(A1,I6,M1,E6,P9);(A1,I6,M1,E6,P10);(A1,I6,M1,E6,P11);(A1,I6,M1,E6,P12);(A1,I6,M1,E6,P13);(A1,I6,M1,E6,P14);(A1,I6,M1,E6,P15);(A1,I6,M1,E6,P16);(A1,I6,M1,E6,P17);(A1,I6,M1,E6,P18);(A1,I6,M1,E6,P19);(A1,I6,M1,E6,P20);(A1,I6,M1,E7,P1);(A1,I6,M1,E7,P2);(A1,I6,M1,E7,P3);(A1,I6,M1,E7,P4);(A1,I6,M1,E7,P5);(A1,I6,M1,E7,P6);(A1,I6,M1,E7,P7);(A1,I6,M1,E7,P8);(A1,I6,M1,E7,P9);(A1,I6,M1,E7,P10);(A1,I6,M1,E7,P11);(A1,I6,M1,E7,P12);(A1,I6,M1,E7,P13);(A1,I6,M1,E7,P14);(A1,I6,M1,E7,P15);(A1,I6,M1,E7,P16);(A1,I6,M1,E7,P17);(A1,I6,M1,E7,P18);(A1,I6,M1,E7,P19);(A1,I6,M1,E7,P20);(A1,I6,M1,E8,P1);(A1,I6,M1,E8,P2);(A1,I6,M1,E8,P3);(A1,I6,M1,E8,P4);(A1,I6,M1,E8,P5);(A1,I6,M1,E8,P6);(A1,I6,M1,E8,P7);(A1,I6,M1,E8,P8);(A1,I6,M1,E8,P9);(A1,I6,M1,E8,P10);(A1,I6,M1,E8,P11);(A1,I6,M1,E8,P12);(A1,I6,M1,E8,P13);(A1,I6,M1,E8,P14);(A1,I6,M1,E8,P15);(A1,I6,M1,E8,P16);(A1,I6,M1,E8,P17);(A1,I6,M1,E8,P18);(A1,I6,M1,E8,P19);(A1,I6,M1,E8,P20);(A1,I6,M1,E9,P1);(A1,I6,M1,E9,P2);(A1,I6,M1,E9,P3);(A1,I6,M1,E9,P4);(A1,I6,M1,E9,P5);(A1,I6,M1,E9,P6);(A1,I6,M1,E9,P7);(A1,I6,M1,E9,P8);(A1,I6,M1,E9,P9);(A1,I6,M1,E9,P10);(A1,I6,M1,E9,P11);(A1,I6,M1,E9,P12);(A1,I6,M1,E9,P13);(A1,I6,M1,E9,P14);(A1,I6,M1,E9,P15);(A1,I6,M1,E9,P16);(A1,I6,M1,E9,P17);(A1,I6,M1,E9,P18);(A1,I6,M1,E9,P19);(A1,I6,M1,E9,P20);(A1,I6,M2,E1,P1);(A1,I6,M2,E1,P2);(A1,I6,M2,E1,P3);(A1,I6,M2,E1,P4);(A1,I6,M2,E1,P5);(A1,I6,M2,E1,P6);(A1,I6,M2,E1,P7);(A1,I6,M2,E1,P8);(A1,I6,M2,E1,P9);(A1,I6,M2,E1,P10);(A1,I6,M2,E1,P11);(A1,I6,M2,E1,P12);(A1,I6,M2,E1,P13);(A1,I6,M2,E1,P14);(A1,I6,M2,E1,P15);(A1,I6,M2,E1,P16);(A1,I6,M2,E1,P17);(A1,I6,M2,E1,P18);(A1,I6,M2,E1,P19);(A1,I6,M2,E1,P20);(A1,I6,M2,E2,P1);(A1,I6,M2,E2,P2);(A1,I6,M2,E2,P3);(A1,I6,M2,E2,P4);(A1,I6,M2,E2,P5);(A1,I6,M2,E2,P6);(A1,I6,M2,E2,P7);(A1,I6,M2,E2,P8);(A1,I6,M2,E2,P9);(A1,I6,M2,E2,P10);(A1,I6,M2,E2,P11);(A1,I6,M2,E2,P12);(A1,I6,M2,E2,P13);(A1,I6,M2,E2,P14);(A1,I6,M2,E2,P15);(A1,I6,M2,E2,P16);(A1,I6,M2,E2,P17);(A1,I6,M2,E2,P18);(A1,I6,M2,E2,P19);(A1,I6,M2,E2,P20);(A1,I6,M2,E3,P1);(A1,I6,M2,E3,P2);(A1,I6,M2,E3,P3);(A1,I6,M2,E3,P4);(A1,I6,M2,E3,P5);(A1,I6,M2,E3,P6);(A1,I6,M2,E3,P7);(A1,I6,M2,E3,P8);(A1,I6,M2,E3,P9);(A1,I6,M2,E3,P10);(A1,I6,M2,E3,P11);(A1,I6,M2,E3,P12);(A1,I6,M2,E3,P13);(A1,I6,M2,E3,P14);(A1,I6,M2,E3,P15);(A1,I6,M2,E3,P16);(A1,I6,M2,E3,P17);(A1,I6,M2,E3,P18);(A1,I6,M2,E3,P19);(A1,I6,M2,E3,P20);(A1,I6,M2,E4,P1);(A1,I6,M2,E4,P2);(A1,I6,M2,E4,P3);(A1,I6,M2,E4,P4);(A1,I6,M2,E4,P5);(A1,I6,M2,E4,P6);(A1,I6,M2,E4,P7);(A1,I6,M2,E4,P8);(A1,I6,M2,E4,P9);(A1,I6,M2,E4,P10);(A1,I6,M2,E4,P11);(A1,I6,M2,E4,P12);(A1,I6,M2,E4,P13);(A1,I6,M2,E4,P14);(A1,I6,M2,E4,P15);(A1,I6,M2,E4,P16);(A1,I6,M2,E4,P17);(A1,I6,M2,E4,P18);(A1,I6,M2,E4,P19);(A1,I6,M2,E4,P20);(A1,I6,M2,E5,P1);(A1,I6,M2,E5,P2);(A1,I6,M2,E5,P3);(A1,I6,M2,E5,P4);(A1,I6,M2,E5,P5);(A1,I6,M2,E5,P6);(A1,I6,M2,E5,P7);(A1,I6,M2,E5,P8);(A1,I6,M2,E5,P9);(A1,I6,M2,E5,P10);(A1,I6,M2,E5,P11);(A1,I6,M2,E5,P12);(A1,I6,M2,E5,P13);(A1,I6,M2,E5,P14);(A1,I6,M2,E5,P15);(A1,I6,M2,E5,P16);(A1,I6,M2,E5,P17);(A1,I6,M2,E5,P18);(A1,I6,M2,E5,P19);(A1,I6,M2,E5,P20);(A1,I6,M2,E6,P1);(A1,I6,M2,E6,P2);(A1,I6,M2,E6,P3);(A1,I6,M2,E6,P4);(A1,I6,M2,E6,P5);(A1,I6,M2,E6,P6);(A1,I6,M2,E6,P7);(A1,I6,M2,E6,P8);(A1,I6,M2,E6,P9);(A1,I6,M2,E6,P10);(A1,I6,M2,E6,P11);(A1,I6,M2,E6,P12);(A1,I6,M2,E6,P13);(A1,I6,M2,E6,P14);(A1,I6,M2,E6,P15);(A1,I6,M2,E6,P16);(A1,I6,M2,E6,P17);(A1,I6,M2,E6,P18);(A1,I6,M2,E6,P19);(A1,I6,M2,E6,P20);(A1,I6,M2,E7,P1);(A1,I6,M2,E7,P2);(A1,I6,M2,E7,P3);(A1,I6,M2,E7,P4);(A1,I6,M2,E7,P5);(A1,I6,M2,E7,P6);(A1,I6,M2,E7,P7);(A1,I6,M2,E7,P8);(A1,I6,M2,E7,P9);(A1,I6,M2,E7,P10);(A1,I6,M2,E7,P11);(A1,I6,M2,E7,P12);(A1,I6,M2,E7,P13);(A1,I6,M2,E7,P14);(A1,I6,M2,E7,P15);(A1,I6,M2,E7,P16);(A1,I6,M2,E7,P17);(A1,I6,M2,E7,P18);(A1,I6,M2,E7,P19);(A1,I6,M2,E7,P20);(A1,I6,M2,E8,P1);(A1,I6,M2,E8,P2);(A1,I6,M2,E8,P3);(A1,I6,M2,E8,P4);(A1,I6,M2,E8,P5);(A1,I6,M2,E8,P6);(A1,I6,M2,E8,P7);(A1,I6,M2,E8,P8);(A1,I6,M2,E8,P9);(A1,I6,M2,E8,P10);(A1,I6,M2,E8,P11);(A1,I6,M2,E8,P12);(A1,I6,M2,E8,P13);(A1,I6,M2,E8,P14);(A1,I6,M2,E8,P15);(A1,I6,M2,E8,P16);(A1,I6,M2,E8,P17);(A1,I6,M2,E8,P18);(A1,I6,M2,E8,P19);(A1,I6,M2,E8,P20);(A1,I6,M2,E9,P1);(A1,I6,M2,E9,P2);(A1,I6,M2,E9,P3);(A1,I6,M2,E9,P4);(A1,I6,M2,E9,P5);(A1,I6,M2,E9,P6);(A1,I6,M2,E9,P7);(A1,I6,M2,E9,P8);(A1,I6,M2,E9,P9);(A1,I6,M2,E9,P10);(A1,I6,M2,E9,P11);(A1,I6,M2,E9,P12);(A1,I6,M2,E9,P13);(A1,I6,M2,E9,P14);(A1,I6,M2,E9,P15);(A1,I6,M2,E9,P16);(A1,I6,M2,E9,P17);(A1,I6,M2,E9,P18);(A1,I6,M2,E9,P19);(A1,I6,M2,E9,P20);(A1,I6,M3,E1,P1);(A1,I6,M3,E1,P2);(A1,I6,M3,E1,P3);(A1,I6,M3,E1,P4);(A1,I6,M3,E1,P5);(A1,I6,M3,E1,P6);(A1,I6,M3,E1,P7);(A1,I6,M3,E1,P8);(A1,I6,M3,E1,P9);(A1,I6,M3,E1,P10);(A1,I6,M3,E1,P11);(A1,I6,M3,E1,P12);(A1,I6,M3,E1,P13);(A1,I6,M3,E1,P14);(A1,I6,M3,E1,P15);(A1,I6,M3,E1,P16);(A1,I6,M3,E1,P17);(A1,I6,M3,E1,P18);(A1,I6,M3,E1,P19);(A1,I6,M3,E1,P20);(A1,I6,M3,E2,P1);(A1,I6,M3,E2,P2);(A1,I6,M3,E2,P3);(A1,I6,M3,E2,P4);(A1,I6,M3,E2,P5);(A1,I6,M3,E2,P6);(A1,I6,M3,E2,P7);(A1,I6,M3,E2,P8);(A1,I6,M3,E2,P9);(A1,I6,M3,E2,P10);(A1,I6,M3,E2,P11);(A1,I6,M3,E2,P12);(A1,I6,M3,E2,P13);(A1,I6,M3,E2,P14);(A1,I6,M3,E2,P15);(A1,I6,M3,E2,P16);(A1,I6,M3,E2,P17);(A1,I6,M3,E2,P18);(A1,I6,M3,E2,P19);(A1,I6,M3,E2,P20);(A1,I6,M3,E3,P1);(A1,I6,M3,E3,P2);(A1,I6,M3,E3,P3);(A1,I6,M3,E3,P4);(A1,I6,M3,E3,P5);(A1,I6,M3,E3,P6);(A1,I6,M3,E3,P7);(A1,I6,M3,E3,P8);(A1,I6,M3,E3,P9);(A1,I6,M3,E3,P10);(A1,I6,M3,E3,P11);(A1,I6,M3,E3,P12);(A1,I6,M3,E3,P13);(A1,I6,M3,E3,P14);(A1,I6,M3,E3,P15);(A1,I6,M3,E3,P16);(A1,I6,M3,E3,P17);(A1,I6,M3,E3,P18);(A1,I6,M3,E3,P19);(A1,I6,M3,E3,P20);(A1,I6,M3,E4,P1);(A1,I6,M3,E4,P2);(A1,I6,M3,E4,P3);(A1,I6,M3,E4,P4);(A1,I6,M3,E4,P5);(A1,I6,M3,E4,P6);(A1,I6,M3,E4,P7);(A1,I6,M3,E4,P8);(A1,I6,M3,E4,P9);(A1,I6,M3,E4,P10);(A1,I6,M3,E4,P11);(A1,I6,M3,E4,P12);(A1,I6,M3,E4,P13);(A1,I6,M3,E4,P14);(A1,I6,M3,E4,P15);(A1,I6,M3,E4,P16);(A1,I6,M3,E4,P17);(A1,I6,M3,E4,P18);(A1,I6,M3,E4,P19);(A1,I6,M3,E4,P20);(A1,I6,M3,E5,P1);(A1,I6,M3,E5,P2);(A1,I6,M3,E5,P3);(A1,I6,M3,E5,P4);(A1,I6,M3,E5,P5);(A1,I6,M3,E5,P6);(A1,I6,M3,E5,P7);(A1,I6,M3,E5,P8);(A1,I6,M3,E5,P9);(A1,I6,M3,E5,P10);(A1,I6,M3,E5,P11);(A1,I6,M3,E5,P12);(A1,I6,M3,E5,P13);(A1,I6,M3,E5,P14);(A1,I6,M3,E5,P15);(A1,I6,M3,E5,P16);(A1,I6,M3,E5,P17);(A1,I6,M3,E5,P18);(A1,I6,M3,E5,P19);(A1,I6,M3,E5,P20);(A1,I6,M3,E6,P1);(A1,I6,M3,E6,P2);(A1,I6,M3,E6,P3);(A1,I6,M3,E6,P4);(A1,I6,M3,E6,P5);(A1,I6,M3,E6,P6);(A1,I6,M3,E6,P7);(A1,I6,M3,E6,P8);(A1,I6,M3,E6,P9);(A1,I6,M3,E6,P10);(A1,I6,M3,E6,P11);(A1,I6,M3,E6,P12);(A1,I6,M3,E6,P13);(A1,I6,M3,E6,P14);(A1,I6,M3,E6,P15);(A1,I6,M3,E6,P16);(A1,I6,M3,E6,P17);(A1,I6,M3,E6,P18);(A1,I6,M3,E6,P19);(A1,I6,M3,E6,P20);(A1,I6,M3,E7,P1);(A1,I6,M3,E7,P2);(A1,I6,M3,E7,P3);(A1,I6,M3,E7,P4);(A1,I6,M3,E7,P5);(A1,I6,M3,E7,P6);(A1,I6,M3,E7,P7);(A1,I6,M3,E7,P8);(A1,I6,M3,E7,P9);(A1,I6,M3,E7,P10);(A1,I6,M3,E7,P11);(A1,I6,M3,E7,P12);(A1,I6,M3,E7,P13);(A1,I6,M3,E7,P14);(A1,I6,M3,E7,P15);(A1,I6,M3,E7,P16);(A1,I6,M3,E7,P17);(A1,I6,M3,E7,P18);(A1,I6,M3,E7,P19);(A1,I6,M3,E7,P20);(A1,I6,M3,E8,P1);(A1,I6,M3,E8,P2);(A1,I6,M3,E8,P3);(A1,I6,M3,E8,P4);(A1,I6,M3,E8,P5);(A1,I6,M3,E8,P6);(A1,I6,M3,E8,P7);(A1,I6,M3,E8,P8);(A1,I6,M3,E8,P9);(A1,I6,M3,E8,P10);(A1,I6,M3,E8,P11);(A1,I6,M3,E8,P12);(A1,I6,M3,E8,P13);(A1,I6,M3,E8,P14);(A1,I6,M3,E8,P15);(A1,I6,M3,E8,P16);(A1,I6,M3,E8,P17);(A1,I6,M3,E8,P18);(A1,I6,M3,E8,P19);(A1,I6,M3,E8,P20);(A1,I6,M3,E9,P1);(A1,I6,M3,E9,P2);(A1,I6,M3,E9,P3);(A1,I6,M3,E9,P4);(A1,I6,M3,E9,P5);(A1,I6,M3,E9,P6);(A1,I6,M3,E9,P7);(A1,I6,M3,E9,P8);(A1,I6,M3,E9,P9);(A1,I6,M3,E9,P10);(A1,I6,M3,E9,P11);(A1,I6,M3,E9,P12);(A1,I6,M3,E9,P13);(A1,I6,M3,E9,P14);(A1,I6,M3,E9,P15);(A1,I6,M3,E9,P16);(A1,I6,M3,E9,P17);(A1,I6,M3,E9,P18);(A1,I6,M3,E9,P19);(A1,I6,M3,E9,P20);(A1,I6,M4,E1,P1);(A1,I6,M4,E1,P2);(A1,I6,M4,E1,P3);(A1,I6,M4,E1,P4);(A1,I6,M4,E1,P5);(A1,I6,M4,E1,P6);(A1,I6,M4,E1,P7);(A1,I6,M4,E1,P8);(A1,I6,M4,E1,P9);(A1,I6,M4,E1,P10);(A1,I6,M4,E1,P11);(A1,I6,M4,E1,P12);(A1,I6,M4,E1,P13);(A1,I6,M4,E1,P14);(A1,I6,M4,E1,P15);(A1,I6,M4,E1,P16);(A1,I6,M4,E1,P17);(A1,I6,M4,E1,P18);(A1,I6,M4,E1,P19);(A1,I6,M4,E1,P20);(A1,I6,M4,E2,P1);(A1,I6,M4,E2,P2);(A1,I6,M4,E2,P3);(A1,I6,M4,E2,P4);(A1,I6,M4,E2,P5);(A1,I6,M4,E2,P6);(A1,I6,M4,E2,P7);(A1,I6,M4,E2,P8);(A1,I6,M4,E2,P9);(A1,I6,M4,E2,P10);(A1,I6,M4,E2,P11);(A1,I6,M4,E2,P12);(A1,I6,M4,E2,P13);(A1,I6,M4,E2,P14);(A1,I6,M4,E2,P15);(A1,I6,M4,E2,P16);(A1,I6,M4,E2,P17);(A1,I6,M4,E2,P18);(A1,I6,M4,E2,P19);(A1,I6,M4,E2,P20);(A1,I6,M4,E3,P1);(A1,I6,M4,E3,P2);(A1,I6,M4,E3,P3);(A1,I6,M4,E3,P4);(A1,I6,M4,E3,P5);(A1,I6,M4,E3,P6);(A1,I6,M4,E3,P7);(A1,I6,M4,E3,P8);(A1,I6,M4,E3,P9);(A1,I6,M4,E3,P10);(A1,I6,M4,E3,P11);(A1,I6,M4,E3,P12);(A1,I6,M4,E3,P13);(A1,I6,M4,E3,P14);(A1,I6,M4,E3,P15);(A1,I6,M4,E3,P16);(A1,I6,M4,E3,P17);(A1,I6,M4,E3,P18);(A1,I6,M4,E3,P19);(A1,I6,M4,E3,P20);(A1,I6,M4,E4,P1);(A1,I6,M4,E4,P2);(A1,I6,M4,E4,P3);(A1,I6,M4,E4,P4);(A1,I6,M4,E4,P5);(A1,I6,M4,E4,P6);(A1,I6,M4,E4,P7);(A1,I6,M4,E4,P8);(A1,I6,M4,E4,P9);(A1,I6,M4,E4,P10);(A1,I6,M4,E4,P11);(A1,I6,M4,E4,P12);(A1,I6,M4,E4,P13);(A1,I6,M4,E4,P14);(A1,I6,M4,E4,P15);(A1,I6,M4,E4,P16);(A1,I6,M4,E4,P17);(A1,I6,M4,E4,P18);(A1,I6,M4,E4,P19);(A1,I6,M4,E4,P20);(A1,I6,M4,E5,P1);(A1,I6,M4,E5,P2);(A1,I6,M4,E5,P3);(A1,I6,M4,E5,P4);(A1,I6,M4,E5,P5);(A1,I6,M4,E5,P6);(A1,I6,M4,E5,P7);(A1,I6,M4,E5,P8);(A1,I6,M4,E5,P9);(A1,I6,M4,E5,P10);(A1,I6,M4,E5,P11);(A1,I6,M4,E5,P12);(A1,I6,M4,E5,P13);(A1,I6,M4,E5,P14);(A1,I6,M4,E5,P15);(A1,I6,M4,E5,P16);(A1,I6,M4,E5,P17);(A1,I6,M4,E5,P18);(A1,I6,M4,E5,P19);(A1,I6,M4,E5,P20);(A1,I6,M4,E6,P1);(A1,I6,M4,E6,P2);(A1,I6,M4,E6,P3);(A1,I6,M4,E6,P4);(A1,I6,M4,E6,P5);(A1,I6,M4,E6,P6);(A1,I6,M4,E6,P7);(A1,I6,M4,E6,P8);(A1,I6,M4,E6,P9);(A1,I6,M4,E6,P10);(A1,I6,M4,E6,P11);(A1,I6,M4,E6,P12);(A1,I6,M4,E6,P13);(A1,I6,M4,E6,P14);(A1,I6,M4,E6,P15);(A1,I6,M4,E6,P16);(A1,I6,M4,E6,P17);(A1,I6,M4,E6,P18);(A1,I6,M4,E6,P19);(A1,I6,M4,E6,P20);(A1,I6,M4,E7,P1);(A1,I6,M4,E7,P2);(A1,I6,M4,E7,P3);(A1,I6,M4,E7,P4);(A1,I6,M4,E7,P5);(A1,I6,M4,E7,P6);(A1,I6,M4,E7,P7);(A1,I6,M4,E7,P8);(A1,I6,M4,E7,P9);(A1,I6,M4,E7,P10);(A1,I6,M4,E7,P11);(A1,I6,M4,E7,P12);(A1,I6,M4,E7,P13);(A1,I6,M4,E7,P14);(A1,I6,M4,E7,P15);(A1,I6,M4,E7,P16);(A1,I6,M4,E7,P17);(A1,I6,M4,E7,P18);(A1,I6,M4,E7,P19);(A1,I6,M4,E7,P20);(A1,I6,M4,E8,P1);(A1,I6,M4,E8,P2);(A1,I6,M4,E8,P3);(A1,I6,M4,E8,P4);(A1,I6,M4,E8,P5);(A1,I6,M4,E8,P6);(A1,I6,M4,E8,P7);(A1,I6,M4,E8,P8);(A1,I6,M4,E8,P9);(A1,I6,M4,E8,P10);(A1,I6,M4,E8,P11);(A1,I6,M4,E8,P12);(A1,I6,M4,E8,P13);(A1,I6,M4,E8,P14);(A1,I6,M4,E8,P15);(A1,I6,M4,E8,P16);(A1,I6,M4,E8,P17);(A1,I6,M4,E8,P18);(A1,I6,M4,E8,P19);(A1,I6,M4,E8,P20);(A1,I6,M4,E9,P1);(A1,I6,M4,E9,P2);(A1,I6,M4,E9,P3);(A1,I6,M4,E9,P4);(A1,I6,M4,E9,P5);(A1,I6,M4,E9,P6);(A1,I6,M4,E9,P7);(A1,I6,M4,E9,P8);(A1,I6,M4,E9,P9);(A1,I6,M4,E9,P10);(A1,I6,M4,E9,P11);(A1,I6,M4,E9,P12);(A1,I6,M4,E9,P13);(A1,I6,M4,E9,P14);(A1,I6,M4,E9,P15);(A1,I6,M4,E9,P16);(A1,I6,M4,E9,P17);(A1,I6,M4,E9,P18);(A1,I6,M4,E9,P19);(A1,I6,M4,E9,P20);(A1,I6,M5,E1,P1);(A1,I6,M5,E1,P2);(A1,I6,M5,E1,P3);(A1,I6,M5,E1,P4);(A1,I6,M5,E1,P5);(A1,I6,M5,E1,P6);(A1,I6,M5,E1,P7);(A1,I6,M5,E1,P8);(A1,I6,M5,E1,P9);(A1,I6,M5,E1,P10);(A1,I6,M5,E1,P11);(A1,I6,M5,E1,P12);(A1,I6,M5,E1,P13);(A1,I6,M5,E1,P14);(A1,I6,M5,E1,P15);(A1,I6,M5,E1,P16);(A1,I6,M5,E1,P17);(A1,I6,M5,E1,P18);(A1,I6,M5,E1,P19);(A1,I6,M5,E1,P20);(A1,I6,M5,E2,P1);(A1,I6,M5,E2,P2);(A1,I6,M5,E2,P3);(A1,I6,M5,E2,P4);(A1,I6,M5,E2,P5);(A1,I6,M5,E2,P6);(A1,I6,M5,E2,P7);(A1,I6,M5,E2,P8);(A1,I6,M5,E2,P9);(A1,I6,M5,E2,P10);(A1,I6,M5,E2,P11);(A1,I6,M5,E2,P12);(A1,I6,M5,E2,P13);(A1,I6,M5,E2,P14);(A1,I6,M5,E2,P15);(A1,I6,M5,E2,P16);(A1,I6,M5,E2,P17);(A1,I6,M5,E2,P18);(A1,I6,M5,E2,P19);(A1,I6,M5,E2,P20);(A1,I6,M5,E3,P1);(A1,I6,M5,E3,P2);(A1,I6,M5,E3,P3);(A1,I6,M5,E3,P4);(A1,I6,M5,E3,P5);(A1,I6,M5,E3,P6);(A1,I6,M5,E3,P7);(A1,I6,M5,E3,P8);(A1,I6,M5,E3,P9);(A1,I6,M5,E3,P10);(A1,I6,M5,E3,P11);(A1,I6,M5,E3,P12);(A1,I6,M5,E3,P13);(A1,I6,M5,E3,P14);(A1,I6,M5,E3,P15);(A1,I6,M5,E3,P16);(A1,I6,M5,E3,P17);(A1,I6,M5,E3,P18);(A1,I6,M5,E3,P19);(A1,I6,M5,E3,P20);(A1,I6,M5,E4,P1);(A1,I6,M5,E4,P2);(A1,I6,M5,E4,P3);(A1,I6,M5,E4,P4);(A1,I6,M5,E4,P5);(A1,I6,M5,E4,P6);(A1,I6,M5,E4,P7);(A1,I6,M5,E4,P8);(A1,I6,M5,E4,P9);(A1,I6,M5,E4,P10);(A1,I6,M5,E4,P11);(A1,I6,M5,E4,P12);(A1,I6,M5,E4,P13);(A1,I6,M5,E4,P14);(A1,I6,M5,E4,P15);(A1,I6,M5,E4,P16);(A1,I6,M5,E4,P17);(A1,I6,M5,E4,P18);(A1,I6,M5,E4,P19);(A1,I6,M5,E4,P20);(A1,I6,M5,E5,P1);(A1,I6,M5,E5,P2);(A1,I6,M5,E5,P3);(A1,I6,M5,E5,P4);(A1,I6,M5,E5,P5);(A1,I6,M5,E5,P6);(A1,I6,M5,E5,P7);(A1,I6,M5,E5,P8);(A1,I6,M5,E5,P9);(A1,I6,M5,E5,P10);(A1,I6,M5,E5,P11);(A1,I6,M5,E5,P12);(A1,I6,M5,E5,P13);(A1,I6,M5,E5,P14);(A1,I6,M5,E5,P15);(A1,I6,M5,E5,P16);(A1,I6,M5,E5,P17);(A1,I6,M5,E5,P18);(A1,I6,M5,E5,P19);(A1,I6,M5,E5,P20);(A1,I6,M5,E6,P1);(A1,I6,M5,E6,P2);(A1,I6,M5,E6,P3);(A1,I6,M5,E6,P4);(A1,I6,M5,E6,P5);(A1,I6,M5,E6,P6);(A1,I6,M5,E6,P7);(A1,I6,M5,E6,P8);(A1,I6,M5,E6,P9);(A1,I6,M5,E6,P10);(A1,I6,M5,E6,P11);(A1,I6,M5,E6,P12);(A1,I6,M5,E6,P13);(A1,I6,M5,E6,P14);(A1,I6,M5,E6,P15);(A1,I6,M5,E6,P16);(A1,I6,M5,E6,P17);(A1,I6,M5,E6,P18);(A1,I6,M5,E6,P19);(A1,I6,M5,E6,P20);(A1,I6,M5,E7,P1);(A1,I6,M5,E7,P2);(A1,I6,M5,E7,P3);(A1,I6,M5,E7,P4);(A1,I6,M5,E7,P5);(A1,I6,M5,E7,P6);(A1,I6,M5,E7,P7);(A1,I6,M5,E7,P8);(A1,I6,M5,E7,P9);(A1,I6,M5,E7,P10);(A1,I6,M5,E7,P11);(A1,I6,M5,E7,P12);(A1,I6,M5,E7,P13);(A1,I6,M5,E7,P14);(A1,I6,M5,E7,P15);(A1,I6,M5,E7,P16);(A1,I6,M5,E7,P17);(A1,I6,M5,E7,P18);(A1,I6,M5,E7,P19);(A1,I6,M5,E7,P20);(A1,I6,M5,E8,P1);(A1,I6,M5,E8,P2);(A1,I6,M5,E8,P3);(A1,I6,M5,E8,P4);(A1,I6,M5,E8,P5);(A1,I6,M5,E8,P6);(A1,I6,M5,E8,P7);(A1,I6,M5,E8,P8);(A1,I6,M5,E8,P9);(A1,I6,M5,E8,P10);(A1,I6,M5,E8,P11);(A1,I6,M5,E8,P12);(A1,I6,M5,E8,P13);(A1,I6,M5,E8,P14);(A1,I6,M5,E8,P15);(A1,I6,M5,E8,P16);(A1,I6,M5,E8,P17);(A1,I6,M5,E8,P18);(A1,I6,M5,E8,P19);(A1,I6,M5,E8,P20);(A1,I6,M5,E9,P1);(A1,I6,M5,E9,P2);(A1,I6,M5,E9,P3);(A1,I6,M5,E9,P4);(A1,I6,M5,E9,P5);(A1,I6,M5,E9,P6);(A1,I6,M5,E9,P7);(A1,I6,M5,E9,P8);(A1,I6,M5,E9,P9);(A1,I6,M5,E9,P10);(A1,I6,M5,E9,P11);(A1,I6,M5,E9,P12);(A1,I6,M5,E9,P13);(A1,I6,M5,E9,P14);(A1,I6,M5,E9,P15);(A1,I6,M5,E9,P16);(A1,I6,M5,E9,P17);(A1,I6,M5,E9,P18);(A1,I6,M5,E9,P19);(A1,I6,M5,E9,P20);(A1,I6,M6,E1,P1);(A1,I6,M6,E1,P2);(A1,I6,M6,E1,P3);(A1,I6,M6,E1,P4);(A1,I6,M6,E1,P5);(A1,I6,M6,E1,P6);(A1,I6,M6,E1,P7);(A1,I6,M6,E1,P8);(A1,I6,M6,E1,P9);(A1,I6,M6,E1,P10);(A1,I6,M6,E1,P11);(A1,I6,M6,E1,P12);(A1,I6,M6,E1,P13);(A1,I6,M6,E1,P14);(A1,I6,M6,E1,P15);(A1,I6,M6,E1,P16);(A1,I6,M6,E1,P17);(A1,I6,M6,E1,P18);(A1,I6,M6,E1,P19);(A1,I6,M6,E1,P20);(A1,I6,M6,E2,P1);(A1,I6,M6,E2,P2);(A1,I6,M6,E2,P3);(A1,I6,M6,E2,P4);(A1,I6,M6,E2,P5);(A1,I6,M6,E2,P6);(A1,I6,M6,E2,P7);(A1,I6,M6,E2,P8);(A1,I6,M6,E2,P9);(A1,I6,M6,E2,P10);(A1,I6,M6,E2,P11);(A1,I6,M6,E2,P12);(A1,I6,M6,E2,P13);(A1,I6,M6,E2,P14);(A1,I6,M6,E2,P15);(A1,I6,M6,E2,P16);(A1,I6,M6,E2,P17);(A1,I6,M6,E2,P18);(A1,I6,M6,E2,P19);(A1,I6,M6,E2,P20);(A1,I6,M6,E3,P1);(A1,I6,M6,E3,P2);(A1,I6,M6,E3,P3);(A1,I6,M6,E3,P4);(A1,I6,M6,E3,P5);(A1,I6,M6,E3,P6);(A1,I6,M6,E3,P7);(A1,I6,M6,E3,P8);(A1,I6,M6,E3,P9);(A1,I6,M6,E3,P10);(A1,I6,M6,E3,P11);(A1,I6,M6,E3,P12);(A1,I6,M6,E3,P13);(A1,I6,M6,E3,P14);(A1,I6,M6,E3,P15);(A1,I6,M6,E3,P16);(A1,I6,M6,E3,P17);(A1,I6,M6,E3,P18);(A1,I6,M6,E3,P19);(A1,I6,M6,E3,P20);(A1,I6,M6,E4,P1);(A1,I6,M6,E4,P2);(A1,I6,M6,E4,P3);(A1,I6,M6,E4,P4);(A1,I6,M6,E4,P5);(A1,I6,M6,E4,P6);(A1,I6,M6,E4,P7);(A1,I6,M6,E4,P8);(A1,I6,M6,E4,P9);(A1,I6,M6,E4,P10);(A1,I6,M6,E4,P11);(A1,I6,M6,E4,P12);(A1,I6,M6,E4,P13);(A1,I6,M6,E4,P14);(A1,I6,M6,E4,P15);(A1,I6,M6,E4,P16);(A1,I6,M6,E4,P17);(A1,I6,M6,E4,P18);(A1,I6,M6,E4,P19);(A1,I6,M6,E4,P20);(A1,I6,M6,E5,P1);(A1,I6,M6,E5,P2);(A1,I6,M6,E5,P3);(A1,I6,M6,E5,P4);(A1,I6,M6,E5,P5);(A1,I6,M6,E5,P6);(A1,I6,M6,E5,P7);(A1,I6,M6,E5,P8);(A1,I6,M6,E5,P9);(A1,I6,M6,E5,P10);(A1,I6,M6,E5,P11);(A1,I6,M6,E5,P12);(A1,I6,M6,E5,P13);(A1,I6,M6,E5,P14);(A1,I6,M6,E5,P15);(A1,I6,M6,E5,P16);(A1,I6,M6,E5,P17);(A1,I6,M6,E5,P18);(A1,I6,M6,E5,P19);(A1,I6,M6,E5,P20);(A1,I6,M6,E6,P1);(A1,I6,M6,E6,P2);(A1,I6,M6,E6,P3);(A1,I6,M6,E6,P4);(A1,I6,M6,E6,P5);(A1,I6,M6,E6,P6);(A1,I6,M6,E6,P7);(A1,I6,M6,E6,P8);(A1,I6,M6,E6,P9);(A1,I6,M6,E6,P10);(A1,I6,M6,E6,P11);(A1,I6,M6,E6,P12);(A1,I6,M6,E6,P13);(A1,I6,M6,E6,P14);(A1,I6,M6,E6,P15);(A1,I6,M6,E6,P16);(A1,I6,M6,E6,P17);(A1,I6,M6,E6,P18);(A1,I6,M6,E6,P19);(A1,I6,M6,E6,P20);(A1,I6,M6,E7,P1);(A1,I6,M6,E7,P2);(A1,I6,M6,E7,P3);(A1,I6,M6,E7,P4);(A1,I6,M6,E7,P5);(A1,I6,M6,E7,P6);(A1,I6,M6,E7,P7);(A1,I6,M6,E7,P8);(A1,I6,M6,E7,P9);(A1,I6,M6,E7,P10);(A1,I6,M6,E7,P11);(A1,I6,M6,E7,P12);(A1,I6,M6,E7,P13);(A1,I6,M6,E7,P14);(A1,I6,M6,E7,P15);(A1,I6,M6,E7,P16);(A1,I6,M6,E7,P17);(A1,I6,M6,E7,P18);(A1,I6,M6,E7,P19);(A1,I6,M6,E7,P20);(A1,I6,M6,E8,P1);(A1,I6,M6,E8,P2);(A1,I6,M6,E8,P3);(A1,I6,M6,E8,P4);(A1,I6,M6,E8,P5);(A1,I6,M6,E8,P6);(A1,I6,M6,E8,P7);(A1,I6,M6,E8,P8);(A1,I6,M6,E8,P9);(A1,I6,M6,E8,P10);(A1,I6,M6,E8,P11);(A1,I6,M6,E8,P12);(A1,I6,M6,E8,P13);(A1,I6,M6,E8,P14);(A1,I6,M6,E8,P15);(A1,I6,M6,E8,P16);(A1,I6,M6,E8,P17);(A1,I6,M6,E8,P18);(A1,I6,M6,E8,P19);(A1,I6,M6,E8,P20);(A1,I6,M6,E9,P1);(A1,I6,M6,E9,P2);(A1,I6,M6,E9,P3);(A1,I6,M6,E9,P4);(A1,I6,M6,E9,P5);(A1,I6,M6,E9,P6);(A1,I6,M6,E9,P7);(A1,I6,M6,E9,P8);(A1,I6,M6,E9,P9);(A1,I6,M6,E9,P10);(A1,I6,M6,E9,P11);(A1,I6,M6,E9,P12);(A1,I6,M6,E9,P13);(A1,I6,M6,E9,P14);(A1,I6,M6,E9,P15);(A1,I6,M6,E9,P16);(A1,I6,M6,E9,P17);(A1,I6,M6,E9,P18);(A1,I6,M6,E9,P19);(A1,I6,M6,E9,P20);(A2,I1,M1,E1,P1);(A2,I1,M1,E1,P2);(A2,I1,M1,E1,P3);(A2,I1,M1,E1,P4);(A2,I1,M1,E1,P5);(A2,I1,M1,E1,P6);(A2,I1,M1,E1,P7);(A2,I1,M1,E1,P8);(A2,I1,M1,E1,P9);(A2,I1,M1,E1,P10);(A2,I1,M1,E1,P11);(A2,I1,M1,E1,P12);(A2,I1,M1,E1,P13);(A2,I1,M1,E1,P14);(A2,I1,M1,E1,P15);(A2,I1,M1,E1,P16);(A2,I1,M1,E1,P17);(A2,I1,M1,E1,P18);(A2,I1,M1,E1,P19);(A2,I1,M1,E1,P20);(A2,I1,M1,E2,P1);(A2,I1,M1,E2,P2);(A2,I1,M1,E2,P3);(A2,I1,M1,E2,P4);(A2,I1,M1,E2,P5);(A2,I1,M1,E2,P6);(A2,I1,M1,E2,P7);(A2,I1,M1,E2,P8);(A2,I1,M1,E2,P9);(A2,I1,M1,E2,P10);(A2,I1,M1,E2,P11);(A2,I1,M1,E2,P12);(A2,I1,M1,E2,P13);(A2,I1,M1,E2,P14);(A2,I1,M1,E2,P15);(A2,I1,M1,E2,P16);(A2,I1,M1,E2,P17);(A2,I1,M1,E2,P18);(A2,I1,M1,E2,P19);(A2,I1,M1,E2,P20);(A2,I1,M1,E3,P1);(A2,I1,M1,E3,P2);(A2,I1,M1,E3,P3);(A2,I1,M1,E3,P4);(A2,I1,M1,E3,P5);(A2,I1,M1,E3,P6);(A2,I1,M1,E3,P7);(A2,I1,M1,E3,P8);(A2,I1,M1,E3,P9);(A2,I1,M1,E3,P10);(A2,I1,M1,E3,P11);(A2,I1,M1,E3,P12);(A2,I1,M1,E3,P13);(A2,I1,M1,E3,P14);(A2,I1,M1,E3,P15);(A2,I1,M1,E3,P16);(A2,I1,M1,E3,P17);(A2,I1,M1,E3,P18);(A2,I1,M1,E3,P19);(A2,I1,M1,E3,P20);(A2,I1,M1,E4,P1);(A2,I1,M1,E4,P2);(A2,I1,M1,E4,P3);(A2,I1,M1,E4,P4);(A2,I1,M1,E4,P5);(A2,I1,M1,E4,P6);(A2,I1,M1,E4,P7);(A2,I1,M1,E4,P8);(A2,I1,M1,E4,P9);(A2,I1,M1,E4,P10);(A2,I1,M1,E4,P11);(A2,I1,M1,E4,P12);(A2,I1,M1,E4,P13);(A2,I1,M1,E4,P14);(A2,I1,M1,E4,P15);(A2,I1,M1,E4,P16);(A2,I1,M1,E4,P17);(A2,I1,M1,E4,P18);(A2,I1,M1,E4,P19);(A2,I1,M1,E4,P20);(A2,I1,M1,E5,P1);(A2,I1,M1,E5,P2);(A2,I1,M1,E5,P3);(A2,I1,M1,E5,P4);(A2,I1,M1,E5,P5);(A2,I1,M1,E5,P6);(A2,I1,M1,E5,P7);(A2,I1,M1,E5,P8);(A2,I1,M1,E5,P9);(A2,I1,M1,E5,P10);(A2,I1,M1,E5,P11);(A2,I1,M1,E5,P12);(A2,I1,M1,E5,P13);(A2,I1,M1,E5,P14);(A2,I1,M1,E5,P15);(A2,I1,M1,E5,P16);(A2,I1,M1,E5,P17);(A2,I1,M1,E5,P18);(A2,I1,M1,E5,P19);(A2,I1,M1,E5,P20);(A2,I1,M1,E6,P1);(A2,I1,M1,E6,P2);(A2,I1,M1,E6,P3);(A2,I1,M1,E6,P4);(A2,I1,M1,E6,P5);(A2,I1,M1,E6,P6);(A2,I1,M1,E6,P7);(A2,I1,M1,E6,P8);(A2,I1,M1,E6,P9);(A2,I1,M1,E6,P10);(A2,I1,M1,E6,P11);(A2,I1,M1,E6,P12);(A2,I1,M1,E6,P13);(A2,I1,M1,E6,P14);(A2,I1,M1,E6,P15);(A2,I1,M1,E6,P16);(A2,I1,M1,E6,P17);(A2,I1,M1,E6,P18);(A2,I1,M1,E6,P19);(A2,I1,M1,E6,P20);(A2,I1,M1,E7,P1);(A2,I1,M1,E7,P2);(A2,I1,M1,E7,P3);(A2,I1,M1,E7,P4);(A2,I1,M1,E7,P5);(A2,I1,M1,E7,P6);(A2,I1,M1,E7,P7);(A2,I1,M1,E7,P8);(A2,I1,M1,E7,P9);(A2,I1,M1,E7,P10);(A2,I1,M1,E7,P11);(A2,I1,M1,E7,P12);(A2,I1,M1,E7,P13);(A2,I1,M1,E7,P14);(A2,I1,M1,E7,P15);(A2,I1,M1,E7,P16);(A2,I1,M1,E7,P17);(A2,I1,M1,E7,P18);(A2,I1,M1,E7,P19);(A2,I1,M1,E7,P20);(A2,I1,M1,E8,P1);(A2,I1,M1,E8,P2);(A2,I1,M1,E8,P3);(A2,I1,M1,E8,P4);(A2,I1,M1,E8,P5);(A2,I1,M1,E8,P6);(A2,I1,M1,E8,P7);(A2,I1,M1,E8,P8);(A2,I1,M1,E8,P9);(A2,I1,M1,E8,P10);(A2,I1,M1,E8,P11);(A2,I1,M1,E8,P12);(A2,I1,M1,E8,P13);(A2,I1,M1,E8,P14);(A2,I1,M1,E8,P15);(A2,I1,M1,E8,P16);(A2,I1,M1,E8,P17);(A2,I1,M1,E8,P18);(A2,I1,M1,E8,P19);(A2,I1,M1,E8,P20);(A2,I1,M1,E9,P1);(A2,I1,M1,E9,P2);(A2,I1,M1,E9,P3);(A2,I1,M1,E9,P4);(A2,I1,M1,E9,P5);(A2,I1,M1,E9,P6);(A2,I1,M1,E9,P7);(A2,I1,M1,E9,P8);(A2,I1,M1,E9,P9);(A2,I1,M1,E9,P10);(A2,I1,M1,E9,P11);(A2,I1,M1,E9,P12);(A2,I1,M1,E9,P13);(A2,I1,M1,E9,P14);(A2,I1,M1,E9,P15);(A2,I1,M1,E9,P16);(A2,I1,M1,E9,P17);(A2,I1,M1,E9,P18);(A2,I1,M1,E9,P19);(A2,I1,M1,E9,P20);(A2,I1,M2,E1,P1);(A2,I1,M2,E1,P2);(A2,I1,M2,E1,P3);(A2,I1,M2,E1,P4);(A2,I1,M2,E1,P5);(A2,I1,M2,E1,P6);(A2,I1,M2,E1,P7);(A2,I1,M2,E1,P8);(A2,I1,M2,E1,P9);(A2,I1,M2,E1,P10);(A2,I1,M2,E1,P11);(A2,I1,M2,E1,P12);(A2,I1,M2,E1,P13);(A2,I1,M2,E1,P14);(A2,I1,M2,E1,P15);(A2,I1,M2,E1,P16);(A2,I1,M2,E1,P17);(A2,I1,M2,E1,P18);(A2,I1,M2,E1,P19);(A2,I1,M2,E1,P20);(A2,I1,M2,E2,P1);(A2,I1,M2,E2,P2);(A2,I1,M2,E2,P3);(A2,I1,M2,E2,P4);(A2,I1,M2,E2,P5);(A2,I1,M2,E2,P6);(A2,I1,M2,E2,P7);(A2,I1,M2,E2,P8);(A2,I1,M2,E2,P9);(A2,I1,M2,E2,P10);(A2,I1,M2,E2,P11);(A2,I1,M2,E2,P12);(A2,I1,M2,E2,P13);(A2,I1,M2,E2,P14);(A2,I1,M2,E2,P15);(A2,I1,M2,E2,P16);(A2,I1,M2,E2,P17);(A2,I1,M2,E2,P18);(A2,I1,M2,E2,P19);(A2,I1,M2,E2,P20);(A2,I1,M2,E3,P1);(A2,I1,M2,E3,P2);(A2,I1,M2,E3,P3);(A2,I1,M2,E3,P4);(A2,I1,M2,E3,P5);(A2,I1,M2,E3,P6);(A2,I1,M2,E3,P7);(A2,I1,M2,E3,P8);(A2,I1,M2,E3,P9);(A2,I1,M2,E3,P10);(A2,I1,M2,E3,P11);(A2,I1,M2,E3,P12);(A2,I1,M2,E3,P13);(A2,I1,M2,E3,P14);(A2,I1,M2,E3,P15);(A2,I1,M2,E3,P16);(A2,I1,M2,E3,P17);(A2,I1,M2,E3,P18);(A2,I1,M2,E3,P19);(A2,I1,M2,E3,P20);(A2,I1,M2,E4,P1);(A2,I1,M2,E4,P2);(A2,I1,M2,E4,P3);(A2,I1,M2,E4,P4);(A2,I1,M2,E4,P5);(A2,I1,M2,E4,P6);(A2,I1,M2,E4,P7);(A2,I1,M2,E4,P8);(A2,I1,M2,E4,P9);(A2,I1,M2,E4,P10);(A2,I1,M2,E4,P11);(A2,I1,M2,E4,P12);(A2,I1,M2,E4,P13);(A2,I1,M2,E4,P14);(A2,I1,M2,E4,P15);(A2,I1,M2,E4,P16);(A2,I1,M2,E4,P17);(A2,I1,M2,E4,P18);(A2,I1,M2,E4,P19);(A2,I1,M2,E4,P20);(A2,I1,M2,E5,P1);(A2,I1,M2,E5,P2);(A2,I1,M2,E5,P3);(A2,I1,M2,E5,P4);(A2,I1,M2,E5,P5);(A2,I1,M2,E5,P6);(A2,I1,M2,E5,P7);(A2,I1,M2,E5,P8);(A2,I1,M2,E5,P9);(A2,I1,M2,E5,P10);(A2,I1,M2,E5,P11);(A2,I1,M2,E5,P12);(A2,I1,M2,E5,P13);(A2,I1,M2,E5,P14);(A2,I1,M2,E5,P15);(A2,I1,M2,E5,P16);(A2,I1,M2,E5,P17);(A2,I1,M2,E5,P18);(A2,I1,M2,E5,P19);(A2,I1,M2,E5,P20);(A2,I1,M2,E6,P1);(A2,I1,M2,E6,P2);(A2,I1,M2,E6,P3);(A2,I1,M2,E6,P4);(A2,I1,M2,E6,P5);(A2,I1,M2,E6,P6);(A2,I1,M2,E6,P7);(A2,I1,M2,E6,P8);(A2,I1,M2,E6,P9);(A2,I1,M2,E6,P10);(A2,I1,M2,E6,P11);(A2,I1,M2,E6,P12);(A2,I1,M2,E6,P13);(A2,I1,M2,E6,P14);(A2,I1,M2,E6,P15);(A2,I1,M2,E6,P16);(A2,I1,M2,E6,P17);(A2,I1,M2,E6,P18);(A2,I1,M2,E6,P19);(A2,I1,M2,E6,P20);(A2,I1,M2,E7,P1);(A2,I1,M2,E7,P2);(A2,I1,M2,E7,P3);(A2,I1,M2,E7,P4);(A2,I1,M2,E7,P5);(A2,I1,M2,E7,P6);(A2,I1,M2,E7,P7);(A2,I1,M2,E7,P8);(A2,I1,M2,E7,P9);(A2,I1,M2,E7,P10);(A2,I1,M2,E7,P11);(A2,I1,M2,E7,P12);(A2,I1,M2,E7,P13);(A2,I1,M2,E7,P14);(A2,I1,M2,E7,P15);(A2,I1,M2,E7,P16);(A2,I1,M2,E7,P17);(A2,I1,M2,E7,P18);(A2,I1,M2,E7,P19);(A2,I1,M2,E7,P20);(A2,I1,M2,E8,P1);(A2,I1,M2,E8,P2);(A2,I1,M2,E8,P3);(A2,I1,M2,E8,P4);(A2,I1,M2,E8,P5);(A2,I1,M2,E8,P6);(A2,I1,M2,E8,P7);(A2,I1,M2,E8,P8);(A2,I1,M2,E8,P9);(A2,I1,M2,E8,P10);(A2,I1,M2,E8,P11);(A2,I1,M2,E8,P12);(A2,I1,M2,E8,P13);(A2,I1,M2,E8,P14);(A2,I1,M2,E8,P15);(A2,I1,M2,E8,P16);(A2,I1,M2,E8,P17);(A2,I1,M2,E8,P18);(A2,I1,M2,E8,P19);(A2,I1,M2,E8,P20);(A2,I1,M2,E9,P1);(A2,I1,M2,E9,P2);(A2,I1,M2,E9,P3);(A2,I1,M2,E9,P4);(A2,I1,M2,E9,P5);(A2,I1,M2,E9,P6);(A2,I1,M2,E9,P7);(A2,I1,M2,E9,P8);(A2,I1,M2,E9,P9);(A2,I1,M2,E9,P10);(A2,I1,M2,E9,P11);(A2,I1,M2,E9,P12);(A2,I1,M2,E9,P13);(A2,I1,M2,E9,P14);(A2,I1,M2,E9,P15);(A2,I1,M2,E9,P16);(A2,I1,M2,E9,P17);(A2,I1,M2,E9,P18);(A2,I1,M2,E9,P19);(A2,I1,M2,E9,P20);(A2,I1,M3,E1,P1);(A2,I1,M3,E1,P2);(A2,I1,M3,E1,P3);(A2,I1,M3,E1,P4);(A2,I1,M3,E1,P5);(A2,I1,M3,E1,P6);(A2,I1,M3,E1,P7);(A2,I1,M3,E1,P8);(A2,I1,M3,E1,P9);(A2,I1,M3,E1,P10);(A2,I1,M3,E1,P11);(A2,I1,M3,E1,P12);(A2,I1,M3,E1,P13);(A2,I1,M3,E1,P14);(A2,I1,M3,E1,P15);(A2,I1,M3,E1,P16);(A2,I1,M3,E1,P17);(A2,I1,M3,E1,P18);(A2,I1,M3,E1,P19);(A2,I1,M3,E1,P20);(A2,I1,M3,E2,P1);(A2,I1,M3,E2,P2);(A2,I1,M3,E2,P3);(A2,I1,M3,E2,P4);(A2,I1,M3,E2,P5);(A2,I1,M3,E2,P6);(A2,I1,M3,E2,P7);(A2,I1,M3,E2,P8);(A2,I1,M3,E2,P9);(A2,I1,M3,E2,P10);(A2,I1,M3,E2,P11);(A2,I1,M3,E2,P12);(A2,I1,M3,E2,P13);(A2,I1,M3,E2,P14);(A2,I1,M3,E2,P15);(A2,I1,M3,E2,P16);(A2,I1,M3,E2,P17);(A2,I1,M3,E2,P18);(A2,I1,M3,E2,P19);(A2,I1,M3,E2,P20);(A2,I1,M3,E3,P1);(A2,I1,M3,E3,P2);(A2,I1,M3,E3,P3);(A2,I1,M3,E3,P4);(A2,I1,M3,E3,P5);(A2,I1,M3,E3,P6);(A2,I1,M3,E3,P7);(A2,I1,M3,E3,P8);(A2,I1,M3,E3,P9);(A2,I1,M3,E3,P10);(A2,I1,M3,E3,P11);(A2,I1,M3,E3,P12);(A2,I1,M3,E3,P13);(A2,I1,M3,E3,P14);(A2,I1,M3,E3,P15);(A2,I1,M3,E3,P16);(A2,I1,M3,E3,P17);(A2,I1,M3,E3,P18);(A2,I1,M3,E3,P19);(A2,I1,M3,E3,P20);(A2,I1,M3,E4,P1);(A2,I1,M3,E4,P2);(A2,I1,M3,E4,P3);(A2,I1,M3,E4,P4);(A2,I1,M3,E4,P5);(A2,I1,M3,E4,P6);(A2,I1,M3,E4,P7);(A2,I1,M3,E4,P8);(A2,I1,M3,E4,P9);(A2,I1,M3,E4,P10);(A2,I1,M3,E4,P11);(A2,I1,M3,E4,P12);(A2,I1,M3,E4,P13);(A2,I1,M3,E4,P14);(A2,I1,M3,E4,P15);(A2,I1,M3,E4,P16);(A2,I1,M3,E4,P17);(A2,I1,M3,E4,P18);(A2,I1,M3,E4,P19);(A2,I1,M3,E4,P20);(A2,I1,M3,E5,P1);(A2,I1,M3,E5,P2);(A2,I1,M3,E5,P3);(A2,I1,M3,E5,P4);(A2,I1,M3,E5,P5);(A2,I1,M3,E5,P6);(A2,I1,M3,E5,P7);(A2,I1,M3,E5,P8);(A2,I1,M3,E5,P9);(A2,I1,M3,E5,P10);(A2,I1,M3,E5,P11);(A2,I1,M3,E5,P12);(A2,I1,M3,E5,P13);(A2,I1,M3,E5,P14);(A2,I1,M3,E5,P15);(A2,I1,M3,E5,P16);(A2,I1,M3,E5,P17);(A2,I1,M3,E5,P18);(A2,I1,M3,E5,P19);(A2,I1,M3,E5,P20);(A2,I1,M3,E6,P1);(A2,I1,M3,E6,P2);(A2,I1,M3,E6,P3);(A2,I1,M3,E6,P4);(A2,I1,M3,E6,P5);(A2,I1,M3,E6,P6);(A2,I1,M3,E6,P7);(A2,I1,M3,E6,P8);(A2,I1,M3,E6,P9);(A2,I1,M3,E6,P10);(A2,I1,M3,E6,P11);(A2,I1,M3,E6,P12);(A2,I1,M3,E6,P13);(A2,I1,M3,E6,P14);(A2,I1,M3,E6,P15);(A2,I1,M3,E6,P16);(A2,I1,M3,E6,P17);(A2,I1,M3,E6,P18);(A2,I1,M3,E6,P19);(A2,I1,M3,E6,P20);(A2,I1,M3,E7,P1);(A2,I1,M3,E7,P2);(A2,I1,M3,E7,P3);(A2,I1,M3,E7,P4);(A2,I1,M3,E7,P5);(A2,I1,M3,E7,P6);(A2,I1,M3,E7,P7);(A2,I1,M3,E7,P8);(A2,I1,M3,E7,P9);(A2,I1,M3,E7,P10);(A2,I1,M3,E7,P11);(A2,I1,M3,E7,P12);(A2,I1,M3,E7,P13);(A2,I1,M3,E7,P14);(A2,I1,M3,E7,P15);(A2,I1,M3,E7,P16);(A2,I1,M3,E7,P17);(A2,I1,M3,E7,P18);(A2,I1,M3,E7,P19);(A2,I1,M3,E7,P20);(A2,I1,M3,E8,P1);(A2,I1,M3,E8,P2);(A2,I1,M3,E8,P3);(A2,I1,M3,E8,P4);(A2,I1,M3,E8,P5);(A2,I1,M3,E8,P6);(A2,I1,M3,E8,P7);(A2,I1,M3,E8,P8);(A2,I1,M3,E8,P9);(A2,I1,M3,E8,P10);(A2,I1,M3,E8,P11);(A2,I1,M3,E8,P12);(A2,I1,M3,E8,P13);(A2,I1,M3,E8,P14);(A2,I1,M3,E8,P15);(A2,I1,M3,E8,P16);(A2,I1,M3,E8,P17);(A2,I1,M3,E8,P18);(A2,I1,M3,E8,P19);(A2,I1,M3,E8,P20);(A2,I1,M3,E9,P1);(A2,I1,M3,E9,P2);(A2,I1,M3,E9,P3);(A2,I1,M3,E9,P4);(A2,I1,M3,E9,P5);(A2,I1,M3,E9,P6);(A2,I1,M3,E9,P7);(A2,I1,M3,E9,P8);(A2,I1,M3,E9,P9);(A2,I1,M3,E9,P10);(A2,I1,M3,E9,P11);(A2,I1,M3,E9,P12);(A2,I1,M3,E9,P13);(A2,I1,M3,E9,P14);(A2,I1,M3,E9,P15);(A2,I1,M3,E9,P16);(A2,I1,M3,E9,P17);(A2,I1,M3,E9,P18);(A2,I1,M3,E9,P19);(A2,I1,M3,E9,P20);(A2,I1,M4,E1,P1);(A2,I1,M4,E1,P2);(A2,I1,M4,E1,P3);(A2,I1,M4,E1,P4);(A2,I1,M4,E1,P5);(A2,I1,M4,E1,P6);(A2,I1,M4,E1,P7);(A2,I1,M4,E1,P8);(A2,I1,M4,E1,P9);(A2,I1,M4,E1,P10);(A2,I1,M4,E1,P11);(A2,I1,M4,E1,P12);(A2,I1,M4,E1,P13);(A2,I1,M4,E1,P14);(A2,I1,M4,E1,P15);(A2,I1,M4,E1,P16);(A2,I1,M4,E1,P17);(A2,I1,M4,E1,P18);(A2,I1,M4,E1,P19);(A2,I1,M4,E1,P20);(A2,I1,M4,E2,P1);(A2,I1,M4,E2,P2);(A2,I1,M4,E2,P3);(A2,I1,M4,E2,P4);(A2,I1,M4,E2,P5);(A2,I1,M4,E2,P6);(A2,I1,M4,E2,P7);(A2,I1,M4,E2,P8);(A2,I1,M4,E2,P9);(A2,I1,M4,E2,P10);(A2,I1,M4,E2,P11);(A2,I1,M4,E2,P12);(A2,I1,M4,E2,P13);(A2,I1,M4,E2,P14);(A2,I1,M4,E2,P15);(A2,I1,M4,E2,P16);(A2,I1,M4,E2,P17);(A2,I1,M4,E2,P18);(A2,I1,M4,E2,P19);(A2,I1,M4,E2,P20);(A2,I1,M4,E3,P1);(A2,I1,M4,E3,P2);(A2,I1,M4,E3,P3);(A2,I1,M4,E3,P4);(A2,I1,M4,E3,P5);(A2,I1,M4,E3,P6);(A2,I1,M4,E3,P7);(A2,I1,M4,E3,P8);(A2,I1,M4,E3,P9);(A2,I1,M4,E3,P10);(A2,I1,M4,E3,P11);(A2,I1,M4,E3,P12);(A2,I1,M4,E3,P13);(A2,I1,M4,E3,P14);(A2,I1,M4,E3,P15);(A2,I1,M4,E3,P16);(A2,I1,M4,E3,P17);(A2,I1,M4,E3,P18);(A2,I1,M4,E3,P19);(A2,I1,M4,E3,P20);(A2,I1,M4,E4,P1);(A2,I1,M4,E4,P2);(A2,I1,M4,E4,P3);(A2,I1,M4,E4,P4);(A2,I1,M4,E4,P5);(A2,I1,M4,E4,P6);(A2,I1,M4,E4,P7);(A2,I1,M4,E4,P8);(A2,I1,M4,E4,P9);(A2,I1,M4,E4,P10);(A2,I1,M4,E4,P11);(A2,I1,M4,E4,P12);(A2,I1,M4,E4,P13);(A2,I1,M4,E4,P14);(A2,I1,M4,E4,P15);(A2,I1,M4,E4,P16);(A2,I1,M4,E4,P17);(A2,I1,M4,E4,P18);(A2,I1,M4,E4,P19);(A2,I1,M4,E4,P20);(A2,I1,M4,E5,P1);(A2,I1,M4,E5,P2);(A2,I1,M4,E5,P3);(A2,I1,M4,E5,P4);(A2,I1,M4,E5,P5);(A2,I1,M4,E5,P6);(A2,I1,M4,E5,P7);(A2,I1,M4,E5,P8);(A2,I1,M4,E5,P9);(A2,I1,M4,E5,P10);(A2,I1,M4,E5,P11);(A2,I1,M4,E5,P12);(A2,I1,M4,E5,P13);(A2,I1,M4,E5,P14);(A2,I1,M4,E5,P15);(A2,I1,M4,E5,P16);(A2,I1,M4,E5,P17);(A2,I1,M4,E5,P18);(A2,I1,M4,E5,P19);(A2,I1,M4,E5,P20);(A2,I1,M4,E6,P1);(A2,I1,M4,E6,P2);(A2,I1,M4,E6,P3);(A2,I1,M4,E6,P4);(A2,I1,M4,E6,P5);(A2,I1,M4,E6,P6);(A2,I1,M4,E6,P7);(A2,I1,M4,E6,P8);(A2,I1,M4,E6,P9);(A2,I1,M4,E6,P10);(A2,I1,M4,E6,P11);(A2,I1,M4,E6,P12);(A2,I1,M4,E6,P13);(A2,I1,M4,E6,P14);(A2,I1,M4,E6,P15);(A2,I1,M4,E6,P16);(A2,I1,M4,E6,P17);(A2,I1,M4,E6,P18);(A2,I1,M4,E6,P19);(A2,I1,M4,E6,P20);(A2,I1,M4,E7,P1);(A2,I1,M4,E7,P2);(A2,I1,M4,E7,P3);(A2,I1,M4,E7,P4);(A2,I1,M4,E7,P5);(A2,I1,M4,E7,P6);(A2,I1,M4,E7,P7);(A2,I1,M4,E7,P8);(A2,I1,M4,E7,P9);(A2,I1,M4,E7,P10);(A2,I1,M4,E7,P11);(A2,I1,M4,E7,P12);(A2,I1,M4,E7,P13);(A2,I1,M4,E7,P14);(A2,I1,M4,E7,P15);(A2,I1,M4,E7,P16);(A2,I1,M4,E7,P17);(A2,I1,M4,E7,P18);(A2,I1,M4,E7,P19);(A2,I1,M4,E7,P20);(A2,I1,M4,E8,P1);(A2,I1,M4,E8,P2);(A2,I1,M4,E8,P3);(A2,I1,M4,E8,P4);(A2,I1,M4,E8,P5);(A2,I1,M4,E8,P6);(A2,I1,M4,E8,P7);(A2,I1,M4,E8,P8);(A2,I1,M4,E8,P9);(A2,I1,M4,E8,P10);(A2,I1,M4,E8,P11);(A2,I1,M4,E8,P12);(A2,I1,M4,E8,P13);(A2,I1,M4,E8,P14);(A2,I1,M4,E8,P15);(A2,I1,M4,E8,P16);(A2,I1,M4,E8,P17);(A2,I1,M4,E8,P18);(A2,I1,M4,E8,P19);(A2,I1,M4,E8,P20);(A2,I1,M4,E9,P1);(A2,I1,M4,E9,P2);(A2,I1,M4,E9,P3);(A2,I1,M4,E9,P4);(A2,I1,M4,E9,P5);(A2,I1,M4,E9,P6);(A2,I1,M4,E9,P7);(A2,I1,M4,E9,P8);(A2,I1,M4,E9,P9);(A2,I1,M4,E9,P10);(A2,I1,M4,E9,P11);(A2,I1,M4,E9,P12);(A2,I1,M4,E9,P13);(A2,I1,M4,E9,P14);(A2,I1,M4,E9,P15);(A2,I1,M4,E9,P16);(A2,I1,M4,E9,P17);(A2,I1,M4,E9,P18);(A2,I1,M4,E9,P19);(A2,I1,M4,E9,P20);(A2,I1,M5,E1,P1);(A2,I1,M5,E1,P2);(A2,I1,M5,E1,P3);(A2,I1,M5,E1,P4);(A2,I1,M5,E1,P5);(A2,I1,M5,E1,P6);(A2,I1,M5,E1,P7);(A2,I1,M5,E1,P8);(A2,I1,M5,E1,P9);(A2,I1,M5,E1,P10);(A2,I1,M5,E1,P11);(A2,I1,M5,E1,P12);(A2,I1,M5,E1,P13);(A2,I1,M5,E1,P14);(A2,I1,M5,E1,P15);(A2,I1,M5,E1,P16);(A2,I1,M5,E1,P17);(A2,I1,M5,E1,P18);(A2,I1,M5,E1,P19);(A2,I1,M5,E1,P20);(A2,I1,M5,E2,P1);(A2,I1,M5,E2,P2);(A2,I1,M5,E2,P3);(A2,I1,M5,E2,P4);(A2,I1,M5,E2,P5);(A2,I1,M5,E2,P6);(A2,I1,M5,E2,P7);(A2,I1,M5,E2,P8);(A2,I1,M5,E2,P9);(A2,I1,M5,E2,P10);(A2,I1,M5,E2,P11);(A2,I1,M5,E2,P12);(A2,I1,M5,E2,P13);(A2,I1,M5,E2,P14);(A2,I1,M5,E2,P15);(A2,I1,M5,E2,P16);(A2,I1,M5,E2,P17);(A2,I1,M5,E2,P18);(A2,I1,M5,E2,P19);(A2,I1,M5,E2,P20);(A2,I1,M5,E3,P1);(A2,I1,M5,E3,P2);(A2,I1,M5,E3,P3);(A2,I1,M5,E3,P4);(A2,I1,M5,E3,P5);(A2,I1,M5,E3,P6);(A2,I1,M5,E3,P7);(A2,I1,M5,E3,P8);(A2,I1,M5,E3,P9);(A2,I1,M5,E3,P10);(A2,I1,M5,E3,P11);(A2,I1,M5,E3,P12);(A2,I1,M5,E3,P13);(A2,I1,M5,E3,P14);(A2,I1,M5,E3,P15);(A2,I1,M5,E3,P16);(A2,I1,M5,E3,P17);(A2,I1,M5,E3,P18);(A2,I1,M5,E3,P19);(A2,I1,M5,E3,P20);(A2,I1,M5,E4,P1);(A2,I1,M5,E4,P2);(A2,I1,M5,E4,P3);(A2,I1,M5,E4,P4);(A2,I1,M5,E4,P5);(A2,I1,M5,E4,P6);(A2,I1,M5,E4,P7);(A2,I1,M5,E4,P8);(A2,I1,M5,E4,P9);(A2,I1,M5,E4,P10);(A2,I1,M5,E4,P11);(A2,I1,M5,E4,P12);(A2,I1,M5,E4,P13);(A2,I1,M5,E4,P14);(A2,I1,M5,E4,P15);(A2,I1,M5,E4,P16);(A2,I1,M5,E4,P17);(A2,I1,M5,E4,P18);(A2,I1,M5,E4,P19);(A2,I1,M5,E4,P20);(A2,I1,M5,E5,P1);(A2,I1,M5,E5,P2);(A2,I1,M5,E5,P3);(A2,I1,M5,E5,P4);(A2,I1,M5,E5,P5);(A2,I1,M5,E5,P6);(A2,I1,M5,E5,P7);(A2,I1,M5,E5,P8);(A2,I1,M5,E5,P9);(A2,I1,M5,E5,P10);(A2,I1,M5,E5,P11);(A2,I1,M5,E5,P12);(A2,I1,M5,E5,P13);(A2,I1,M5,E5,P14);(A2,I1,M5,E5,P15);(A2,I1,M5,E5,P16);(A2,I1,M5,E5,P17);(A2,I1,M5,E5,P18);(A2,I1,M5,E5,P19);(A2,I1,M5,E5,P20);(A2,I1,M5,E6,P1);(A2,I1,M5,E6,P2);(A2,I1,M5,E6,P3);(A2,I1,M5,E6,P4);(A2,I1,M5,E6,P5);(A2,I1,M5,E6,P6);(A2,I1,M5,E6,P7);(A2,I1,M5,E6,P8);(A2,I1,M5,E6,P9);(A2,I1,M5,E6,P10);(A2,I1,M5,E6,P11);(A2,I1,M5,E6,P12);(A2,I1,M5,E6,P13);(A2,I1,M5,E6,P14);(A2,I1,M5,E6,P15);(A2,I1,M5,E6,P16);(A2,I1,M5,E6,P17);(A2,I1,M5,E6,P18);(A2,I1,M5,E6,P19);(A2,I1,M5,E6,P20);(A2,I1,M5,E7,P1);(A2,I1,M5,E7,P2);(A2,I1,M5,E7,P3);(A2,I1,M5,E7,P4);(A2,I1,M5,E7,P5);(A2,I1,M5,E7,P6);(A2,I1,M5,E7,P7);(A2,I1,M5,E7,P8);(A2,I1,M5,E7,P9);(A2,I1,M5,E7,P10);(A2,I1,M5,E7,P11);(A2,I1,M5,E7,P12);(A2,I1,M5,E7,P13);(A2,I1,M5,E7,P14);(A2,I1,M5,E7,P15);(A2,I1,M5,E7,P16);(A2,I1,M5,E7,P17);(A2,I1,M5,E7,P18);(A2,I1,M5,E7,P19);(A2,I1,M5,E7,P20);(A2,I1,M5,E8,P1);(A2,I1,M5,E8,P2);(A2,I1,M5,E8,P3);(A2,I1,M5,E8,P4);(A2,I1,M5,E8,P5);(A2,I1,M5,E8,P6);(A2,I1,M5,E8,P7);(A2,I1,M5,E8,P8);(A2,I1,M5,E8,P9);(A2,I1,M5,E8,P10);(A2,I1,M5,E8,P11);(A2,I1,M5,E8,P12);(A2,I1,M5,E8,P13);(A2,I1,M5,E8,P14);(A2,I1,M5,E8,P15);(A2,I1,M5,E8,P16);(A2,I1,M5,E8,P17);(A2,I1,M5,E8,P18);(A2,I1,M5,E8,P19);(A2,I1,M5,E8,P20);(A2,I1,M5,E9,P1);(A2,I1,M5,E9,P2);(A2,I1,M5,E9,P3);(A2,I1,M5,E9,P4);(A2,I1,M5,E9,P5);(A2,I1,M5,E9,P6);(A2,I1,M5,E9,P7);(A2,I1,M5,E9,P8);(A2,I1,M5,E9,P9);(A2,I1,M5,E9,P10);(A2,I1,M5,E9,P11);(A2,I1,M5,E9,P12);(A2,I1,M5,E9,P13);(A2,I1,M5,E9,P14);(A2,I1,M5,E9,P15);(A2,I1,M5,E9,P16);(A2,I1,M5,E9,P17);(A2,I1,M5,E9,P18);(A2,I1,M5,E9,P19);(A2,I1,M5,E9,P20);(A2,I1,M6,E1,P1);(A2,I1,M6,E1,P2);(A2,I1,M6,E1,P3);(A2,I1,M6,E1,P4);(A2,I1,M6,E1,P5);(A2,I1,M6,E1,P6);(A2,I1,M6,E1,P7);(A2,I1,M6,E1,P8);(A2,I1,M6,E1,P9);(A2,I1,M6,E1,P10);(A2,I1,M6,E1,P11);(A2,I1,M6,E1,P12);(A2,I1,M6,E1,P13);(A2,I1,M6,E1,P14);(A2,I1,M6,E1,P15);(A2,I1,M6,E1,P16);(A2,I1,M6,E1,P17);(A2,I1,M6,E1,P18);(A2,I1,M6,E1,P19);(A2,I1,M6,E1,P20);(A2,I1,M6,E2,P1);(A2,I1,M6,E2,P2);(A2,I1,M6,E2,P3);(A2,I1,M6,E2,P4);(A2,I1,M6,E2,P5);(A2,I1,M6,E2,P6);(A2,I1,M6,E2,P7);(A2,I1,M6,E2,P8);(A2,I1,M6,E2,P9);(A2,I1,M6,E2,P10);(A2,I1,M6,E2,P11);(A2,I1,M6,E2,P12);(A2,I1,M6,E2,P13);(A2,I1,M6,E2,P14);(A2,I1,M6,E2,P15);(A2,I1,M6,E2,P16);(A2,I1,M6,E2,P17);(A2,I1,M6,E2,P18);(A2,I1,M6,E2,P19);(A2,I1,M6,E2,P20);(A2,I1,M6,E3,P1);(A2,I1,M6,E3,P2);(A2,I1,M6,E3,P3);(A2,I1,M6,E3,P4);(A2,I1,M6,E3,P5);(A2,I1,M6,E3,P6);(A2,I1,M6,E3,P7);(A2,I1,M6,E3,P8);(A2,I1,M6,E3,P9);(A2,I1,M6,E3,P10);(A2,I1,M6,E3,P11);(A2,I1,M6,E3,P12);(A2,I1,M6,E3,P13);(A2,I1,M6,E3,P14);(A2,I1,M6,E3,P15);(A2,I1,M6,E3,P16);(A2,I1,M6,E3,P17);(A2,I1,M6,E3,P18);(A2,I1,M6,E3,P19);(A2,I1,M6,E3,P20);(A2,I1,M6,E4,P1);(A2,I1,M6,E4,P2);(A2,I1,M6,E4,P3);(A2,I1,M6,E4,P4);(A2,I1,M6,E4,P5);(A2,I1,M6,E4,P6);(A2,I1,M6,E4,P7);(A2,I1,M6,E4,P8);(A2,I1,M6,E4,P9);(A2,I1,M6,E4,P10);(A2,I1,M6,E4,P11);(A2,I1,M6,E4,P12);(A2,I1,M6,E4,P13);(A2,I1,M6,E4,P14);(A2,I1,M6,E4,P15);(A2,I1,M6,E4,P16);(A2,I1,M6,E4,P17);(A2,I1,M6,E4,P18);(A2,I1,M6,E4,P19);(A2,I1,M6,E4,P20);(A2,I1,M6,E5,P1);(A2,I1,M6,E5,P2);(A2,I1,M6,E5,P3);(A2,I1,M6,E5,P4);(A2,I1,M6,E5,P5);(A2,I1,M6,E5,P6);(A2,I1,M6,E5,P7);(A2,I1,M6,E5,P8);(A2,I1,M6,E5,P9);(A2,I1,M6,E5,P10);(A2,I1,M6,E5,P11);(A2,I1,M6,E5,P12);(A2,I1,M6,E5,P13);(A2,I1,M6,E5,P14);(A2,I1,M6,E5,P15);(A2,I1,M6,E5,P16);(A2,I1,M6,E5,P17);(A2,I1,M6,E5,P18);(A2,I1,M6,E5,P19);(A2,I1,M6,E5,P20);(A2,I1,M6,E6,P1);(A2,I1,M6,E6,P2);(A2,I1,M6,E6,P3);(A2,I1,M6,E6,P4);(A2,I1,M6,E6,P5);(A2,I1 ,M6,E6,P6);(A2,I1,M6,E6,P7);(A2,I1,M6,E6,P8);(A2,I1,M6,E6,P9);(A2,I1,M6,E6,P10);(A2,I1, M6,E6,P11);(A2,I1,M6,E6,P12);(A2,I1,M6,E6,P13);(A2,I1,M6,E6,P14);(A2,I1,M6,E6,P15);(A2,I1,M6,E6,P16);(A2,I1,M6,E6,P17);(A2,I1,M6,E6,P18);(A2,I1,M6,E6,P19);(A2,I1,M6,E6,P20);(A2,I1,M6,E7,P1);(A2,I1,M6,E7,P2);(A2,I1,M6,E7,P3);(A2,I1,M6,E7,P4);(A2,I1,M6,E7,P5);(A2,I1,M6,E7,P6);(A2,I1,M6,E7,P7);(A2,I1,M6,E7,P8);(A2,I1,M6,E7,P9);(A2,I1,M6,E7,P10);(A2,I1,M6,E7,P11);(A2,I1,M6,E7,P12);(A2,I1,M6,E7,P13);(A2,I1,M6,E7,P14);(A2,I1,M6,E7,P15);(A2,I1,M6,E7,P16);(A2,I1,M6,E7,P17);(A2,I1,M6,E7,P18);(A2,I1,M6,E7,P19);(A2,I1,M6,E7,P20);(A2,I1,M6,E8,P1);(A2,I1,M6,E8,P2);(A2,I1,M6,E8,P3);(A2,I1,M6,E8,P4);(A2,I1,M6,E8,P5);(A2,I1,M6,E8,P6);(A2,I1,M6,E8,P7);(A2,I1,M6,E8,P8);(A2,I1,M6,E8,P9);(A2,I1,M6,E8,P10);(A2,I1,M6,E8,P11);(A2,I1,M6,E8,P12);(A2,I1,M6,E8,P13);(A2,I1,M6,E8,P14);(A2,I1,M6,E8,P15);(A2,I1,M6,E8,P16);(A2,I1,M6,E8,P17);(A2,I1,M6,E8,P18);(A2,I1,M6,E8,P19);(A2,I1,M6,E8,P20);(A2,I1,M6,E9,P1);(A2,I1,M6,E9,P2);(A2,I1,M6,E9,P3);(A2,I1,M6,E9,P4);(A2,I1,M6,E9,P5);(A2,I1,M6,E9,P6);(A2,I1,M6,E9,P7);(A2,I1,M6,E9,P8);(A2,I1,M6,E9,P9);(A2,I1,M6,E9,P10);(A2,I1,M6,E9,P11);(A2,I1,M6,E9,P12);(A2,I1,M6,E9,P13);(A2,I1,M6,E9,P14);(A2,I1,M6,E9,P15);(A2,I1,M6,E9,P16);(A2,I1,M6,E9,P17);(A2,I1,M6,E9,P18);(A2,I1,M6,E9,P19);(A2,I1,M6,E9,P20);(A2,I2,M1,E1,P1);(A2,I2,M1,E1,P2);(A2,I2,M1,E1,P3);(A2,I2,M1,E1,P4);(A2,I2,M1,E1,P5);(A2,I2,M1,E1,P6);(A2,I2,M1,E1,P7);(A2,I2,M1,E1,P8);(A2,I2,M1,E1,P9);(A2,I2,M1,E1,P10);(A2,I2,M1,E1,P11);(A2,I2,M1,E1,P12);(A2,I2,M1,E1,P13);(A2,I2,M1,E1,P14);(A2,I2,M1,E1,P15);(A2,I2,M1,E1,P16);(A2,I2,M1,E1,P17);(A2,I2,M1,E1,P18);(A2,I2,M1,E1,P19);(A2,I2,M1,E1,P20);(A2,I2,M1,E2,P1);(A2,I2,M1,E2,P2);(A2,I2,M1,E2,P3);(A2,I2,M1,E2,P4);(A2,I2,M1,E2,P5);(A2,I2,M1,E2,P6);(A2,I2,M1,E2,P7);(A2,I2,M1,E2,P8);(A2,I2,M1,E2,P9);(A2,I2,M1,E2,P10);(A2,I2,M1,E2,P11);(A2,I2,M1,E2,P12);(A2,I2,M1,E2,P13);(A2,I2,M1,E2,P14);(A2,I2,M1,E2,P15);(A2,I2,M1,E2,P16);(A2,I2,M1,E2,P17);(A2,I2,M1,E2,P18);(A2,I2,M1,E2,P19);(A2,I2,M1,E2,P20);(A2,I2,M1,E3,P1);(A2,I2,M1,E3,P2);(A2,I2,M1,E3,P3);(A2,I2,M1,E3,P4);(A2,I2,M1,E3,P5);(A2,I2,M1,E3,P6);(A2,I2,M1,E3,P7);(A2,I2,M1,E3,P8);(A2,I2,M1,E3,P9);(A2,I2,M1,E3,P10);(A2,I2,M1,E3,P11);(A2,I2,M1,E3,P12);(A2,I2,M1,E3,P13);(A2,I2,M1,E3,P14);(A2,I2,M1,E3,P15);(A2,I2,M1,E3,P16);(A2,I2,M1,E3,P17);(A2,I2,M1,E3,P18);(A2,I2,M1,E3,P19);(A2,I2,M1,E3,P20);(A2,I2,M1,E4,P1);(A2,I2,M1,E4,P2);(A2,I2,M1,E4,P3);(A2,I2,M1,E4,P4);(A2,I2,M1,E4,P5);(A2,I2,M1,E4,P6);(A2,I2,M1,E4,P7);(A2,I2,M1,E4,P8);(A2,I2,M1,E4,P9);(A2,I2,M1,E4,P10);(A2,I2,M1,E4,P11);(A2,I2,M1,E4,P12);(A2,I2,M1,E4,P13);(A2,I2,M1,E4,P14);(A2,I2,M1,E4,P15);(A2,I2,M1,E4,P16);(A2,I2,M1,E4,P17);(A2,I2,M1,E4,P18);(A2,I2,M1,E4,P19);(A2,I2,M1,E4,P20);(A2,I2,M1,E5,P1);(A2,I2,M1,E5,P2);(A2,I2,M1,E5,P3);(A2,I2,M1,E5,P4);(A2,I2,M1,E5,P5);(A2,I2,M1,E5,P6);(A2,I2,M1,E5,P7);(A2,I2,M1,E5,P8);(A2,I2,M1,E5,P9);(A2,I2,M1,E5,P10);(A2,I2,M1,E5,P11);(A2,I2,M1,E5,P12);(A2,I2,M1,E5,P13);(A2,I2,M1,E5,P14);(A2,I2,M1,E5,P15);(A2,I2,M1,E5,P16);(A2,I2,M1,E5,P17);(A2,I2,M1,E5,P18);(A2,I2,M1,E5,P19);(A2,I2,M1,E5,P20);(A2,I2,M1,E6,P1);(A2,I2,M1,E6,P2);(A2,I2,M1,E6,P3);(A2,I2,M1,E6,P4);(A2,I2,M1,E6,P5);(A2,I2,M1,E6,P6);(A2,I2,M1,E6,P7);(A2,I2,M1,E6,P8);(A2,I2,M1,E6,P9);(A2,I2,M1,E6,P10);(A2,I2,M1,E6,P11);(A2,I2,M1,E6,P12);(A2,I2,M1,E6,P13);(A2,I2,M1,E6,P14);(A2,I2,M1,E6,P15);(A2,I2,M1,E6,P16);(A2,I2,M1,E6,P17);(A2,I2,M1,E6,P18);(A2,I2,M1,E6,P19);(A2,I2,M1,E6,P20);(A2,I2,M1,E7,P1);(A2,I2,M1,E7,P2);(A2,I2,M1,E7,P3);(A2,I2,M1,E7,P4);(A2,I2,M1,E7,P5);(A2,I2,M1,E7,P6);(A2,I2,M1,E7,P7);(A2,I2,M1,E7,P8);(A2,I2,M1,E7,P9);(A2,I2,M1,E7,P10);(A2,I2,M1,E7,P11);(A2,I2,M1,E7,P12);(A2,I2,M1,E7,P13);(A2,I2,M1,E7,P14);(A2,I2,M1,E7,P15);(A2,I2,M1,E7,P16);(A2,I2,M1,E7,P17);(A2,I2,M1,E7,P18);(A2,I2,M1,E7,P19);(A2,I2,M1,E7,P20);(A2,I2,M1,E8,P1);(A2,I2,M1,E8,P2);(A2,I2,M1,E8,P3);(A2,I2,M1,E8,P4);(A2,I2,M1,E8,P5);(A2,I2,M1,E8,P6);(A2,I2,M1,E8,P7);(A2,I2,M1,E8,P8);(A2,I2,M1,E8,P9);(A2,I2,M1,E8,P10);(A2,I2,M1,E8,P11);(A2,I2,M1,E8,P12);(A2,I2,M1,E8,P13);(A2,I2,M1,E8,P14);(A2,I2,M1,E8,P15);(A2,I2,M1,E8,P16);(A2,I2,M1,E8,P17);(A2,I2,M1,E8,P18);(A2,I2,M1,E8,P19);(A2,I2,M1,E8,P20);(A2,I2,M1,E9,P1);(A2,I2,M1,E9,P2);(A2,I2,M1,E9,P3);(A2,I2,M1,E9,P4);(A2,I2,M1,E9,P5);(A2,I2,M1,E9,P6);(A2,I2,M1,E9,P7);(A2,I2,M1,E9,P8);(A2,I2,M1,E9,P9);(A2,I2,M1,E9,P10);(A2,I2,M1,E9,P11);(A2,I2,M1,E9,P12);(A2,I2,M1,E9,P13);(A2,I2,M1,E9,P14);(A2,I2,M1,E9,P15);(A2,I2,M1,E9,P16);(A2,I2,M1,E9,P17);(A2,I2,M1,E9,P18);(A2,I2,M1,E9,P19);(A2,I2,M1,E9,P20);(A2,I2,M2,E1,P1);(A2,I2,M2,E1,P2);(A2,I2,M2,E1,P3);(A2,I2,M2,E1,P4);(A2,I2,M2,E1,P5);(A2,I2,M2,E1,P6);(A2,I2,M2,E1,P7);(A2,I2,M2,E1,P8);(A2,I2,M2,E1,P9);(A2,I2,M2,E1,P10);(A2,I2,M2,E1,P11);(A2,I2,M2,E1,P12);(A2,I2,M2,E1,P13);(A2,I2,M2,E1,P14);(A2,I2,M2,E1,P15);(A2,I2,M2,E1,P16);(A2,I2,M2,E1,P17);(A2,I2,M2,E1,P18);(A2,I2,M2,E1,P19);(A2,I2,M2,E1,P20);(A2,I2,M2,E2,P1);(A2,I2,M2,E2,P2);(A2,I2,M2,E2,P3);(A2,I2,M2,E2,P4);(A2,I2,M2,E2,P5);(A2,I2,M2,E2,P6);(A2,I2,M2,E2,P7);(A2,I2,M2,E2,P8);(A2,I2,M2,E2,P9);(A2,I2,M2,E2,P10);(A2,I2,M2,E2,P11);(A2,I2,M2,E2,P12);(A2,I2,M2,E2,P13);(A2,I2,M2,E2,P14);(A2,I2,M2,E2,P15);(A2,I2,M2,E2,P16);(A2,I2,M2,E2,P17);(A2,I2,M2,E2,P18);(A2,I2,M2,E2,P19);(A2,I2,M2,E2,P20);(A2,I2,M2,E3,P1);(A2,I2,M2,E3,P2);(A2,I2,M2,E3,P3);(A2,I2,M2,E3,P4);(A2,I2,M2,E3,P5);(A2,I2,M2,E3,P6);(A2,I2,M2,E3,P7);(A2,I2,M2,E3,P8);(A2,I2,M2,E3,P9);(A2,I2,M2,E3,P10);(A2,I2,M2,E3,P11);(A2,I2,M2,E3,P12);(A2,I2,M2,E3,P13);(A2,I2,M2,E3,P14);(A2,I2,M2,E3,P15);(A2,I2,M2,E3,P16);(A2,I2,M2,E3,P17);(A2,I2,M2,E3,P18);(A2,I2,M2,E3,P19);(A2,I2,M2,E3,P20);(A2,I2,M2,E4,P1);(A2,I2,M2,E4,P2);(A2,I2,M2,E4,P3);(A2,I2,M2,E4,P4);(A2,I2,M2,E4,P5);(A2,I2,M2,E4,P6);(A2,I2,M2,E4,P7);(A2,I2,M2,E4,P8);(A2,I2,M2,E4,P9);(A2,I2,M2,E4,P10);(A2,I2,M2,E4,P11);(A2,I2,M2,E4,P12);(A2,I2,M2,E4,P13);(A2,I2,M2,E4,P14);(A2,I2,M2,E4,P15);(A2,I2,M2,E4,P16);(A2,I2,M2,E4,P17);(A2,I2,M2,E4,P18);(A2,I2,M2,E4,P19);(A2,I2,M2,E4,P20);(A2,I2,M2,E5,P1);(A2,I2,M2,E5,P2);(A2,I2,M2,E5,P3);(A2,I2,M2,E5,P4);(A2,I2,M2,E5,P5);(A2,I2,M2,E5,P6);(A2,I2,M2,E5,P7);(A2,I2,M2,E5,P8);(A2,I2,M2,E5,P9);(A2,I2,M2,E5,P10);(A2,I2,M2,E5,P11);(A2,I2,M2,E5,P12);(A2,I2,M2,E5,P13);(A2,I2,M2,E5,P14);(A2,I2,M2,E5,P15);(A2,I2,M2,E5,P16);(A2,I2,M2,E5,P17);(A2,I2,M2,E5,P18);(A2,I2,M2,E5,P19);(A2,I2,M2,E5,P20);(A2,I2,M2,E6,P1);(A2,I2,M2,E6,P2);(A2,I2,M2,E6,P3);(A2,I2,M2,E6,P4);(A2,I2,M2,E6,P5);(A2,I2,M2,E6,P6);(A2,I2,M2,E6,P7);(A2,I2,M2,E6,P8);(A2,I2,M2,E6,P9);(A2,I2,M2,E6,P10);(A2,I2,M2,E6,P11);(A2,I2,M2,E6,P12);(A2,I2,M2,E6,P13);(A2,I2,M2,E6,P14);(A2,I2,M2,E6,P15);(A2,I2,M2,E6,P16);(A2,I2,M2,E6,P17);(A2,I2,M2,E6,P18);(A2,I2,M2,E6,P19);(A2,I2,M2,E6,P20);(A2,I2,M2,E7,P1);(A2,I2,M2,E7,P2);(A2,I2,M2,E7,P3);(A2,I2,M2,E7,P4);(A2,I2,M2,E7,P5);(A2,I2,M2,E7,P6);(A2,I2,M2,E7,P7);(A2,I2,M2,E7,P8);(A2,I2,M2,E7,P9);(A2,I2,M2,E7,P10);(A2,I2,M2,E7,P11);(A2,I2,M2,E7,P12);(A2,I2,M2,E7,P13);(A2,I2,M2,E7,P14);(A2,I2,M2,E7,P15);(A2,I2,M2,E7,P16);(A2,I2,M2,E7,P17);(A2,I2,M2,E7,P18);(A2,I2,M2,E7,P19);(A2,I2,M2,E7,P20);(A2,I2,M2,E8,P1);(A2,I2,M2,E8,P2);(A2,I2,M2,E8,P3);(A2,I2,M2,E8,P4);(A2,I2,M2,E8,P5);(A2,I2,M2,E8,P6);(A2,I2,M2,E8,P7);(A2,I2,M2,E8,P8);(A2,I2,M2,E8,P9);(A2,I2,M2,E8,P10);(A2,I2,M2,E8,P11);(A2,I2,M2,E8,P12);(A2,I2,M2,E8,P13);(A2,I2,M2,E8,P14);(A2,I2,M2,E8,P15);(A2,I2,M2,E8,P16);(A2,I2,M2,E8,P17);(A2,I2,M2,E8,P18);(A2,I2,M2,E8,P19);(A2,I2,M2,E8,P20);(A2,I2,M2,E9,P1);(A2,I2,M2,E9,P2);(A2,I2,M2,E9,P3);(A2,I2,M2,E9,P4);(A2,I2,M2,E9,P5);(A2,I2,M2,E9,P6);(A2,I2,M2,E9,P7);(A2,I2,M2,E9,P8);(A2,I2,M2,E9,P9);(A2,I2,M2,E9,P10);(A2,I2,M2,E9,P11);(A2,I2,M2,E9,P12);(A2,I2,M2,E9,P13);(A2,I2,M2,E9,P14);(A2,I2,M2,E9,P15);(A2,I2,M2,E9,P16);(A2,I2,M2,E9,P17);(A2,I2,M2,E9,P18);(A2,I2,M2,E9,P19);(A2,I2,M2,E9,P20);(A2,I2,M3,E1,P1);(A2,I2,M3,E1,P2);(A2,I2,M3,E1,P3);(A2,I2,M3,E1,P4);(A2,I2,M3,E1,P5);(A2,I2,M3,E1,P6);(A2,I2,M3,E1,P7);(A2,I2,M3,E1,P8);(A2,I2,M3,E1,P9);(A2,I2,M3,E1,P10);(A2,I2,M3,E1,P11);(A2,I2,M3,E1,P12);(A2,I2,M3,E1,P13);(A2,I2,M3,E1,P14);(A2,I2,M3,E1,P15);(A2,I2,M3,E1,P16);(A2,I2,M3,E1,P17);(A2,I2,M3,E1,P18);(A2,I2,M3,E1,P19);(A2,I2,M3,E1,P20);(A2,I2,M3,E2,P1);(A2,I2,M3,E2,P2);(A2,I2,M3,E2,P3);(A2,I2,M3,E2,P4);(A2,I2,M3,E2,P5);(A2,I2,M3,E2,P6);(A2,I2,M3,E2,P7);(A2,I2,M3,E2,P8);(A2,I2,M3,E2,P9);(A2,I2,M3,E2,P10);(A2,I2,M3,E2,P11);(A2,I2,M3,E2,P12);(A2,I2,M3,E2,P13);(A2,I2,M3,E2,P14);(A2,I2,M3,E2,P15);(A2,I2,M3,E2,P16);(A2,I2,M3,E2,P17);(A2,I2,M3,E2,P18);(A2,I2,M3,E2,P19);(A2,I2,M3,E2,P20);(A2,I2,M3,E3,P1);(A2,I2,M3,E3,P2);(A2,I2,M3,E3,P3);(A2,I2,M3,E3,P4);(A2,I2,M3,E3,P5);(A2,I2,M3,E3,P6);(A2,I2,M3,E3,P7);(A2,I2,M3,E3,P8);(A2,I2,M3,E3,P9);(A2,I2,M3,E3,P10);(A2,I2,M3,E3,P11);(A2,I2,M3,E3,P12);(A2,I2,M3,E3,P13);(A2,I2,M3,E3,P14);(A2,I2,M3,E3,P15);(A2,I2,M3,E3,P16);(A2,I2,M3,E3,P17);(A2,I2,M3,E3,P18);(A2,I2,M3,E3,P19);(A2,I2,M3,E3,P20);(A2,I2,M3,E4,P1);(A2,I2,M3,E4,P2);(A2,I2,M3,E4,P3);(A2,I2,M3,E4,P4);(A2,I2,M3,E4,P5);(A2,I2,M3,E4,P6);(A2,I2,M3,E4,P7);(A2,I2,M3,E4,P8);(A2,I2,M3,E4,P9);(A2,I2,M3,E4,P10);(A2,I2,M3,E4,P11);(A2,I2,M3,E4,P12);(A2,I2,M3,E4,P13);(A2,I2,M3,E4,P14);(A2,I2,M3,E4,P15);(A2,I2,M3,E4,P16);(A2,I2,M3,E4,P17);(A2,I2,M3,E4,P18);(A2,I2,M3,E4,P19);(A2,I2,M3,E4,P20);(A2,I2,M3,E5,P1);(A2,I2,M3,E5,P2);(A2,I2,M3,E5,P3);(A2,I2,M3,E5,P4);(A2,I2,M3,E5,P5);(A2,I2,M3,E5,P6);(A2,I2,M3,E5,P7);(A2,I2,M3,E5,P8);(A2,I2,M3,E5,P9);(A2,I2,M3,E5,P10);(A2,I2,M3,E5,P11);(A2,I2,M3,E5,P12);(A2,I2,M3,E5,P13);(A2,I2,M3,E5,P14);(A2,I2,M3,E5,P15);(A2,I2,M3,E5,P16);(A2,I2,M3,E5,P17);(A2,I2,M3,E5,P18);(A2,I2,M3,E5,P19);(A2,I2,M3,E5,P20);(A2,I2,M3,E6,P1);(A2,I2,M3,E6,P2);(A2,I2,M3,E6,P3);(A2,I2,M3,E6,P4);(A2,I2,M3,E6,P5);(A2,I2,M3,E6,P6);(A2,I2,M3,E6,P7);(A2,I2,M3,E6,P8);(A2,I2,M3,E6,P9);(A2,I2,M3,E6,P10);(A2,I2,M3,E6,P11);(A2,I2,M3,E6,P12);(A2,I2,M3,E6,P13);(A2,I2,M3,E6,P14);(A2,I2,M3,E6,P15);(A2,I2,M3,E6,P16);(A2,I2,M3,E6,P17);(A2,I2,M3,E6,P18);(A2,I2,M3,E6,P19);(A2,I2,M3,E6,P20);(A2,I2,M3,E7,P1);(A2,I2,M3,E7,P2);(A2,I2,M3,E7,P3);(A2,I2,M3,E7,P4);(A2,I2,M3,E7,P5);(A2,I2,M3,E7,P6);(A2,I2,M3,E7,P7);(A2,I2,M3,E7,P8);(A2,I2,M3,E7,P9);(A2,I2,M3,E7,P10);(A2,I2,M3,E7,P11);(A2,I2,M3,E7,P12);(A2,I2,M3,E7,P13);(A2,I2,M3,E7,P14);(A2,I2,M3,E7,P15);(A2,I2,M3,E7,P16);(A2,I2,M3,E7,P17);(A2,I2,M3,E7,P18);(A2,I2,M3,E7,P19);(A2,I2,M3,E7,P20);(A2,I2,M3,E8,P1);(A2,I2,M3,E8,P2);(A2,I2,M3,E8,P3);(A2,I2,M3,E8,P4);(A2,I2,M3,E8,P5);(A2,I2,M3,E8,P6);(A2,I2,M3,E8,P7);(A2,I2,M3,E8,P8);(A2,I2,M3,E8,P9);(A2,I2,M3,E8,P10);(A2,I2,M3,E8,P11);(A2,I2,M3,E8,P12);(A2,I2,M3,E8,P13);(A2,I2,M3,E8,P14);(A2,I2,M3,E8,P15);(A2,I2,M3,E8,P16);(A2,I2,M3,E8,P17);(A2,I2,M3,E8,P18);(A2,I2,M3,E8,P19);(A2,I2,M3,E8,P20);(A2,I2,M3,E9,P1);(A2,I2,M3,E9,P2);(A2,I2,M3,E9,P3);(A2,I2,M3,E9,P4);(A2,I2,M3,E9,P5);(A2,I2,M3,E9,P6);(A2,I2,M3,E9,P7);(A2,I2,M3,E9,P8);(A2,I2,M3,E9,P9);(A2,I2,M3,E9,P10);(A2,I2,M3,E9,P11);(A2,I2,M3,E9,P12);(A2,I2,M3,E9,P13);(A2,I2,M3,E9,P14);(A2,I2,M3,E9,P15);(A2,I2,M3,E9,P16);(A2,I2,M3,E9,P17);(A2,I2,M3,E9,P18);(A2,I2,M3,E9,P19);(A2,I2,M3,E9,P20);(A2,I2,M4,E1,P1);(A2,I2,M4,E1,P2);(A2,I2,M4,E1,P3);(A2,I2,M4,E1,P4);(A2,I2,M4,E1,P5);(A2,I2,M4,E1,P6);(A2,I2,M4,E1,P7);(A2,I2,M4,E1,P8);(A2,I2,M4,E1,P9);(A2,I2,M4,E1,P10);(A2,I2,M4,E1,P11);(A2,I2,M4,E1,P12);(A2,I2,M4,E1,P13);(A2,I2,M4,E1,P14);(A2,I2,M4,E1,P15);(A2,I2,M4,E1,P16);(A2,I2,M4,E1,P17);(A2,I2,M4,E1,P18);(A2,I2,M4,E1,P19);(A2,I2,M4,E1,P20);(A2,I2,M4,E2,P1);(A2,I2,M4,E2,P2);(A2,I2,M4,E2,P3);(A2,I2,M4,E2,P4);(A2,I2,M4,E2,P5);(A2,I2,M4,E2,P6);(A2,I2,M4,E2,P7);(A2,I2,M4,E2,P8);(A2,I2,M4,E2,P9);(A2,I2,M4,E2,P10);(A2,I2,M4,E2,P11);(A2,I2,M4,E2,P12);(A2,I2,M4,E2,P13);(A2,I2,M4,E2,P14);(A2,I2,M4,E2,P15);(A2,I2,M4,E2,P16);(A2,I2,M4,E2,P17);(A2,I2,M4,E2,P18);(A2,I2,M4,E2,P19);(A2,I2,M4,E2,P20);(A2,I2,M4,E3,P1);(A2,I2,M4,E3,P2);(A2,I2,M4,E3,P3);(A2,I2,M4,E3,P4);(A2,I2,M4,E3,P5);(A2,I2,M4,E3,P6);(A2,I2,M4,E3,P7);(A2,I2,M4,E3,P8);(A2,I2,M4,E3,P9);(A2,I2,M4,E3,P10);(A2,I2,M4,E3,P11);(A2,I2,M4,E3,P12);(A2,I2,M4,E3,P13);(A2,I2,M4,E3,P14);(A2,I2,M4,E3,P15);(A2,I2,M4,E3,P16);(A2,I2,M4,E3,P17);(A2,I2,M4,E3,P18);(A2,I2,M4,E3,P19);(A2,I2,M4,E3,P20);(A2,I2,M4,E4,P1);(A2,I2,M4,E4,P2);(A2,I2,M4,E4,P3);(A2,I2,M4,E4,P4);(A2,I2,M4,E4,P5);(A2,I2,M4,E4,P6);(A2,I2,M4,E4,P7);(A2,I2,M4,E4,P8);(A2,I2,M4,E4,P9);(A2,I2,M4,E4,P10);(A2,I2,M4,E4,P11);(A2,I2,M4,E4,P12);(A2,I2,M4,E4,P13);(A2,I2,M4,E4,P14);(A2,I2,M4,E4,P15);(A2,I2,M4,E4,P16);(A2,I2,M4,E4,P17);(A2,I2,M4,E4,P18);(A2,I2,M4,E4,P19);(A2,I2,M4,E4,P20);(A2,I2,M4,E5,P1);(A2,I2,M4,E5,P2);(A2,I2,M4,E5,P3);(A2,I2,M4,E5,P4);(A2,I2,M4,E5,P5);(A2,I2,M4,E5,P6);(A2,I2,M4,E5,P7);(A2,I2,M4,E5,P8);(A2,I2,M4,E5,P9);(A2,I2,M4,E5,P10);(A2,I2,M4,E5,P11);(A2,I2,M4,E5,P12);(A2,I2,M4,E5,P13);(A2,I2,M4,E5,P14);(A2,I2,M4,E5,P15);(A2,I2,M4,E5,P16);(A2,I2,M4,E5,P17);(A2,I2,M4,E5,P18);(A2,I2,M4,E5,P19);(A2,I2,M4,E5,P20);(A2,I2,M4,E6,P1);(A2,I2,M4,E6,P2);(A2,I2,M4,E6,P3);(A2,I2,M4,E6,P4);(A2,I2,M4,E6,P5);(A2,I2,M4,E6,P6);(A2,I2,M4,E6,P7);(A2,I2,M4,E6,P8);(A2,I2,M4,E6,P9);(A2,I2,M4,E6,P10);(A2,I2,M4,E6,P11);(A2,I2,M4,E6,P12);(A2,I2,M4,E6,P13);(A2,I2,M4,E6,P14);(A2,I2,M4,E6,P15);(A2,I2,M4,E6,P16);(A2,I2,M4,E6,P17);(A2,I2,M4,E6,P18);(A2,I2,M4,E6,P19);(A2,I2,M4,E6,P20);(A2,I2,M4,E7,P1);(A2,I2,M4,E7,P2);(A2,I2,M4,E7,P3);(A2,I2,M4,E7,P4);(A2,I2,M4,E7,P5);(A2,I2,M4,E7,P6);(A2,I2,M4,E7,P7);(A2,I2,M4,E7,P8);(A2,I2,M4,E7,P9);(A2,I2,M4,E7,P10);(A2,I2,M4,E7,P11);(A2,I2,M4,E7,P12);(A2,I2,M4,E7,P13);(A2,I2,M4,E7,P14);(A2,I2,M4,E7,P15);(A2,I2,M4,E7,P16);(A2,I2,M4,E7,P17);(A2,I2,M4,E7,P18);(A2,I2,M4,E7,P19);(A2,I2,M4,E7,P20);(A2,I2,M4,E8,P1);(A2,I2,M4,E8,P2);(A2,I2,M4,E8,P3);(A2,I2,M4,E8,P4);(A2,I2,M4,E8,P5);(A2,I2,M4,E8,P6);(A2,I2,M4,E8,P7);(A2,I2,M4,E8,P8);(A2,I2,M4,E8,P9);(A2,I2,M4,E8,P10);(A2,I2,M4,E8,P11);(A2,I2,M4,E8,P12);(A2,I2,M4,E8,P13);(A2,I2,M4,E8,P14);(A2,I2,M4,E8,P15);(A2,I2,M4,E8,P16);(A2,I2,M4,E8,P17);(A2,I2,M4,E8,P18);(A2,I2,M4,E8,P19);(A2,I2,M4,E8,P20);(A2,I2,M4,E9,P1);(A2,I2,M4,E9,P2);(A2,I2,M4,E9,P3);(A2,I2,M4,E9,P4);(A2,I2,M4,E9,P5);(A2,I2,M4,E9,P6);(A2,I2,M4,E9,P7);(A2,I2,M4,E9,P8);(A2,I2,M4,E9,P9);(A2,I2,M4,E9,P10);(A2,I2,M4,E9,P11);(A2,I2,M4,E9,P12);(A2,I2,M4,E9,P13);(A2,I2,M4,E9,P14);(A2,I2,M4,E9,P15);(A2,I2,M4,E9,P16);(A2,I2,M4,E9,P17);(A2,I2,M4,E9,P18);(A2,I2,M4,E9,P19);(A2,I2,M4,E9,P20);(A2,I2,M5,E1,P1);(A2,I2,M5,E1,P2);(A2,I2,M5,E1,P3);(A2,I2,M5,E1,P4);(A2,I2,M5,E1,P5);(A2,I2,M5,E1,P6);(A2,I2,M5,E1,P7);(A2,I2,M5,E1,P8);(A2,I2,M5,E1,P9);(A2,I2,M5,E1,P10);(A2,I2,M5,E1,P11);(A2,I2,M5,E1,P12);(A2,I2,M5,E1,P13);(A2,I2,M5,E1,P14);(A2,I2,M5,E1,P15);(A2,I2,M5,E1,P16);(A2,I2,M5,E1,P17);(A2,I2,M5,E1,P18);(A2,I2,M5,E1,P19);(A2,I2,M5,E1,P20);(A2,I2,M5,E2,P1);(A2,I2,M5,E2,P2);(A2,I2,M5,E2,P3);(A2,I2,M5,E2,P4);(A2,I2,M5,E2,P5);(A2,I2,M5,E2,P6);(A2,I2,M5,E2,P7);(A2,I2,M5,E2,P8);(A2,I2,M5,E2,P9);(A2,I2,M5,E2,P10);(A2,I2,M5,E2,P11);(A2,I2,M5,E2,P12);(A2,I2,M5,E2,P13);(A2,I2,M5,E2,P14);(A2,I2,M5,E2,P15);(A2,I2,M5,E2,P16);(A2,I2,M5,E2,P17);(A2,I2,M5,E2,P18);(A2,I2,M5,E2,P19);(A2,I2,M5,E2,P20);(A2,I2,M5,E3,P1);(A2,I2,M5,E3,P2);(A2,I2,M5,E3,P3);(A2,I2,M5,E3,P4);(A2,I2,M5,E3,P5);(A2,I2,M5,E3,P6);(A2,I2,M5,E3,P7);(A2,I2,M5,E3,P8);(A2,I2,M5,E3,P9);(A2,I2,M5,E3,P10);(A2,I2,M5,E3,P11);(A2,I2,M5,E3,P12);(A2,I2,M5,E3,P13);(A2,I2,M5,E3,P14);(A2,I2,M5,E3,P15);(A2,I2,M5,E3,P16);(A2,I2,M5,E3,P17);(A2,I2,M5,E3,P18);(A2,I2,M5,E3,P19);(A2,I2,M5,E3,P20);(A2,I2,M5,E4,P1);(A2,I2,M5,E4,P2);(A2,I2,M5,E4,P3);(A2,I2,M5,E4,P4);(A2,I2,M5,E4,P5);(A2,I2,M5,E4,P6);(A2,I2,M5,E4,P7);(A2,I2,M5,E4,P8);(A2,I2,M5,E4,P9);(A2,I2,M5,E4,P10);(A2,I2,M5,E4,P11);(A2,I2,M5,E4,P12);(A2,I2,M5,E4,P13);(A2,I2,M5,E4,P14);(A2,I2,M5,E4,P15);(A2,I2,M5,E4,P16);(A2,I2,M5,E4,P17);(A2,I2,M5,E4,P18);(A2,I2,M5,E4,P19);(A2,I2,M5,E4,P20);(A2,I2,M5,E5,P1);(A2,I2,M5,E5,P2);(A2,I2,M5,E5,P3);(A2,I2,M5,E5,P4);(A2,I2,M5,E5,P5);(A2,I2,M5,E5,P6);(A2,I2,M5,E5,P7);(A2,I2,M5,E5,P8);(A2,I2,M5,E5,P9);(A2,I2,M5,E5,P10);(A2,I2,M5,E5,P11);(A2,I2,M5,E5,P12);(A2,I2,M5,E5,P13);(A2,I2,M5,E5,P14);(A2,I2,M5,E5,P15);(A2,I2,M5,E5,P16);(A2,I2,M5,E5,P17);(A2,I2,M5,E5,P18);(A2,I2,M5,E5,P19);(A2,I2,M5,E5,P20);(A2,I2,M5,E6,P1);(A2,I2,M5,E6,P2);(A2,I2,M5,E6,P3);(A2,I2,M5,E6,P4);(A2,I2,M5,E6,P5);(A2,I2,M5,E6,P6);(A2,I2,M5,E6,P7);(A2,I2,M5,E6,P8);(A2,I2,M5,E6,P9);(A2,I2,M5,E6,P10);(A2,I2,M5,E6,P11);(A2,I2,M5,E6,P12);(A2,I2,M5,E6,P13);(A2,I2,M5,E6,P14);(A2,I2,M5,E6,P15);(A2,I2,M5,E6,P16);(A2,I2,M5,E6,P17);(A2,I2,M5,E6,P18);(A2,I2,M5,E6,P19);(A2,I2,M5,E6,P20);(A2,I2,M5,E7,P1);(A2,I2,M5,E7,P2);(A2,I2,M5,E7,P3);(A2,I2,M5,E7,P4);(A2,I2,M5,E7,P5);(A2,I2,M5,E7,P6);(A2,I2,M5,E7,P7);(A2,I2,M5,E7,P8);(A2,I2,M5,E7,P9);(A2,I2,M5,E7,P10);(A2,I2,M5,E7,P11);(A2,I2,M5,E7,P12);(A2,I2,M5,E7,P13);(A2,I2,M5,E7,P14);(A2,I2,M5,E7,P15);(A2,I2,M5,E7,P16);(A2,I2,M5,E7,P17);(A2,I2,M5,E7,P18);(A2,I2,M5,E7,P19);(A2,I2,M5,E7,P20);(A2,I2,M5,E8,P1);(A2,I2,M5,E8,P2);(A2,I2,M5,E8,P3);(A2,I2,M5,E8,P4);(A2,I2,M5,E8,P5);(A2,I2,M5,E8,P6);(A2,I2,M5,E8,P7);(A2,I2,M5,E8,P8);(A2,I2,M5,E8,P9);(A2,I2,M5,E8,P10);(A2,I2,M5,E8,P11);(A2,I2,M5,E8,P12);(A2,I2,M5,E8,P13);(A2,I2,M5,E8,P14);(A2,I2,M5,E8,P15);(A2,I2,M5,E8,P16);(A2,I2,M5,E8,P17);(A2,I2,M5,E8,P18);(A2,I2,M5,E8,P19);(A2,I2,M5,E8,P20);(A2,I2,M5,E9,P1);(A2,I2,M5,E9,P2);(A2,I2,M5,E9,P3);(A2,I2,M5,E9,P4);(A2,I2,M5,E9,P5);(A2,I2,M5,E9,P6);(A2,I2,M5,E9,P7);(A2,I2,M5,E9,P8);(A2,I2,M5,E9,P9);(A2,I2,M5,E9,P10);(A2,I2,M5,E9,P11);(A2,I2,M5,E9,P12);(A2,I2,M5,E9,P13);(A2,I2,M5,E9,P14);(A2,I2,M5,E9,P15);(A2,I2,M5,E9,P16);(A2,I2,M5,E9,P17);(A2,I2,M5,E9,P18);(A2,I2,M5,E9,P19);(A2,I2,M5,E9,P20);(A2,I2,M6,E1,P1);(A2,I2,M6,E1,P2);(A2,I2,M6,E1,P3);(A2,I2,M6,E1,P4);(A2,I2,M6,E1,P5);(A2,I2,M6,E1,P6);(A2,I2,M6,E1,P7);(A2,I2,M6,E1,P8);(A2,I2,M6,E1,P9);(A2,I2,M6,E1,P10);(A2,I2,M6,E1,P11);(A2,I2,M6,E1,P12);(A2,I2,M6,E1,P13);(A2,I2,M6,E1,P14);(A2,I2,M6,E1,P15);(A2,I2,M6,E1,P16);(A2,I2,M6,E1,P17);(A2,I2,M6,E1,P18);(A2,I2,M6,E1,P19);(A2,I2,M6,E1,P20);(A2,I2,M6,E2,P1);(A2,I2,M6,E2,P2);(A2,I2,M6,E2,P3);(A2,I2,M6,E2,P4);(A2,I2,M6,E2,P5);(A2,I2,M6,E2,P6);(A2,I2,M6,E2,P7);(A2,I2,M6,E2,P8);(A2,I2,M6,E2,P9);(A2,I2,M6,E2,P10);(A2,I2,M6,E2,P11);(A2,I2,M6,E2,P12);(A2,I2,M6,E2,P13);(A2,I2,M6,E2,P14);(A2,I2,M6,E2,P15);(A2,I2,M6,E2,P16);(A2,I2,M6,E2,P17);(A2,I2,M6,E2,P18);(A2,I2,M6,E2,P19);(A2,I2,M6,E2,P20);(A2,I2,M6,E3,P1);(A2,I2,M6,E3,P2);(A2,I2,M6,E3,P3);(A2,I2,M6,E3,P4);(A2,I2,M6,E3,P5);(A2,I2,M6,E3,P6);(A2,I2,M6,E3,P7);(A2,I2,M6,E3,P8);(A2,I2,M6,E3,P9);(A2,I2,M6,E3,P10);(A2,I2,M6,E3,P11);(A2,I2,M6,E3,P12);(A2,I2,M6,E3,P13);(A2,I2,M6,E3,P14);(A2,I2,M6,E3,P15);(A2,I2,M6,E3,P16);(A2,I2,M6,E3,P17);(A2,I2,M6,E3,P18);(A2,I2,M6,E3,P19);(A2,I2,M6,E3,P20);(A2,I2,M6,E4,P1);(A2,I2,M6,E4,P2);(A2,I2,M6,E4,P3);(A2,I2,M6,E4,P4);(A2,I2,M6,E4,P5);(A2,I2,M6,E4,P6);(A2,I2,M6,E4,P7);(A2,I2,M6,E4,P8);(A2,I2,M6,E4,P9);(A2,I2,M6,E4,P10);(A2,I2,M6,E4,P11);(A2,I2,M6,E4,P12);(A2,I2,M6,E4,P13);(A2,I2,M6,E4,P14);(A2,I2,M6,E4,P15);(A2,I2,M6,E4,P16);(A2,I2,M6,E4,P17);(A2,I2,M6,E4,P18);(A2,I2,M6,E4,P19);(A2,I2,M6,E4,P20);(A2,I2,M6,E5,P1);(A2,I2,M6,E5,P2);(A2,I2,M6,E5,P3);(A2,I2,M6,E5,P4);(A2,I2,M6,E5,P5);(A2,I2,M6,E5,P6);(A2,I2,M6,E5,P7);(A2,I2,M6,E5,P8);(A2,I2,M6,E5,P9);(A2,I2,M6,E5,P10);(A2,I2,M6,E5,P11);(A2,I2,M6,E5,P12);(A2,I2,M6,E5,P13);(A2,I2,M6,E5,P14);(A2,I2,M6,E5,P15);(A2,I2,M6,E5,P16);(A2,I2,M6,E5,P17);(A2,I2,M6,E5,P18);(A2,I2,M6,E5,P19);(A2,I2,M6,E5,P20);(A2,I2,M6,E6,P1);(A2,I2,M6,E6,P2);(A2,I2,M6,E6,P3);(A2,I2,M6,E6,P4);(A2,I2,M6,E6,P5);(A2,I2,M6,E6,P6);(A2,I2,M6,E6,P7);(A2,I2,M6,E6,P8);(A2,I2,M6,E6,P9);(A2,I2,M6,E6,P10);(A2,I2,M6,E6,P11);(A2,I2,M6,E6,P12);(A2,I2,M6,E6,P13);(A2,I2,M6,E6,P14);(A2,I2,M6,E6,P15);(A2,I2,M6,E6,P16);(A2,I2,M6,E6,P17);(A2,I2,M6,E6,P18);(A2,I2,M6,E6,P19);(A2,I2,M6,E6,P20);(A2,I2,M6,E7,P1);(A2,I2,M6,E7,P2);(A2,I2,M6,E7,P3);(A2,I2,M6,E7,P4);(A2,I2,M6,E7,P5);(A2,I2,M6,E7,P6);(A2,I2,M6,E7,P7);(A2,I2,M6,E7,P8);(A2,I2,M6,E7,P9);(A2,I2,M6,E7,P10);(A2,I2,M6,E7,P11);(A2,I2,M6,E7,P12);(A2,I2,M6,E7,P13);(A2,I2,M6,E7,P14);(A2,I2,M6,E7,P15);(A2,I2,M6,E7,P16);(A2,I2,M6,E7,P17);(A2,I2,M6,E7,P18);(A2,I2,M6,E7,P19);(A2,I2,M6,E7,P20);(A2,I2,M6,E8,P1);(A2,I2,M6,E8,P2);(A2,I2,M6,E8,P3);(A2,I2,M6,E8,P4);(A2,I2,M6,E8,P5);(A2,I2,M6,E8,P6);(A2,I2,M6,E8,P7);(A2,I2,M6,E8,P8);(A2,I2,M6,E8,P9);(A2,I2,M6,E8,P10);(A2,I2,M6,E8,P11);(A2,I2,M6,E8,P12);(A2,I2,M6,E8,P13);(A2,I2,M6,E8,P14);(A2,I2,M6,E8,P15);(A2,I2,M6,E8,P16);(A2,I2,M6,E8,P17);(A2,I2,M6,E8,P18);(A2,I2,M6,E8,P19);(A2,I2,M6,E8,P20);(A2,I2,M6,E9,P1);(A2,I2,M6,E9,P2);(A2,I2,M6,E9,P3);(A2,I2,M6,E9,P4);(A2,I2,M6,E9,P5);(A2,I2,M6,E9,P6);(A2,I2,M6,E9,P7);(A2,I2,M6,E9,P8);(A2,I2,M6,E9,P9);(A2,I2,M6,E9,P10);(A2,I2,M6,E9,P11);(A2,I2,M6,E9,P12);(A2,I2,M6,E9,P13);(A2,I2,M6,E9,P14);(A2,I2,M6,E9,P15);(A2,I2,M6,E9,P16);(A2,I2,M6,E9,P17);(A2,I2,M6,E9,P18);(A2,I2,M6,E9,P19);(A2,I2,M6,E9,P20);(A2,I3,M1,E1,P1);(A2,I3,M1,E1,P2);(A2,I3,M1,E1,P3);(A2,I3,M1,E1,P4);(A2,I3,M1,E1,P5);(A2,I3,M1,E1,P6);(A2,I3,M1,E1,P7);(A2,I3,M1,E1,P8);(A2,I3,M1,E1,P9);(A2,I3,M1,E1,P10);(A2,I3,M1,E1,P11);(A2,I3,M1,E1,P12);(A2,I3,M1,E1,P13);(A2,I3,M1,E1,P14);(A2,I3,M1,E1,P15);(A2,I3,M1,E1,P16);(A2,I3,M1,E1,P17);(A2,I3,M1,E1,P18);(A2,I3,M1,E1,P19);(A2,I3,M1,E1,P20);(A2,I3,M1,E2,P1);(A2,I3,M1,E2,P2);(A2,I3,M1,E2,P3);(A2,I3,M1,E2,P4);(A2,I3,M1,E2,P5);(A2,I3,M1,E2,P6);(A2,I3,M1,E2,P7);(A2,I3,M1,E2,P8);(A2,I3,M1,E2,P9);(A2,I3,M1,E2,P10);(A2,I3,M1,E2,P11);(A2,I3,M1,E2,P12);(A2,I3,M1,E2,P13);(A2,I3,M1,E2,P14);(A2,I3,M1,E2,P15);(A2,I3,M1,E2,P16);(A2,I3,M1,E2,P17);(A2,I3,M1,E2,P18);(A2,I3,M1,E2,P19);(A2,I3,M1,E2,P20);(A2,I3,M1,E3,P1);(A2,I3,M1,E3,P2);(A2,I3,M1,E3,P3);(A2,I3,M1,E3,P4);(A2,I3,M1,E3,P5);(A2,I3,M1,E3,P6);(A2,I3,M1,E3,P7);(A2,I3,M1,E3,P8);(A2,I3,M1,E3,P9);(A2,I3,M1,E3,P10);(A2,I3,M1,E3,P11);(A2,I3,M1,E3,P12);(A2,I3,M1,E3,P13);(A2,I3,M1,E3,P14);(A2,I3,M1,E3,P15);(A2,I3,M1,E3,P16);(A2,I3,M1,E3,P17);(A2,I3,M1,E3,P18);(A2,I3,M1,E3,P19);(A2,I3,M1,E3,P20);(A2,I3,M1,E4,P1);(A2,I3,M1,E4,P2);(A2,I3,M1,E4,P3);(A2,I3,M1,E4,P4);(A2,I3,M1,E4,P5);(A2,I3,M1,E4,P6);(A2,I3,M1,E4,P7);(A2,I3,M1,E4,P8);(A2,I3,M1,E4,P9);(A2,I3,M1,E4,P10);(A2,I3,M1,E4,P11);(A2,I3,M1,E4,P12);(A2,I3,M1,E4,P13);(A2,I3,M1,E4,P14);(A2,I3,M1,E4,P15);(A2,I3,M1,E4,P16);(A2,I3,M1,E4,P17);(A2,I3,M1,E4,P18);(A2,I3,M1,E4,P19);(A2,I3,M1,E4,P20);(A2,I3,M1,E5,P1);(A2,I3,M1,E5,P2);(A2,I3,M1,E5,P3);(A2,I3,M1,E5,P4);(A2,I3,M1,E5,P5);(A2,I3,M1,E5,P6);(A2,I3,M1,E5,P7);(A2,I3,M1,E5,P8);(A2,I3,M1,E5,P9);(A2,I3,M1,E5,P10);(A2,I3,M1,E5,P11);(A2,I3,M1,E5,P12);(A2,I3,M1,E5,P13);(A2,I3,M1,E5,P14);(A2,I3,M1,E5,P15);(A2,I3,M1,E5,P16);(A2,I3,M1,E5,P17);(A2,I3,M1,E5,P18);(A2,I3,M1,E5,P19);(A2,I3,M1,E5,P20);(A2,I3,M1,E6,P1);(A2,I3,M1,E6,P2);(A2,I3,M1,E6,P3);(A2,I3,M1,E6,P4);(A2,I3,M1,E6,P5);(A2,I3,M1,E6,P6);(A2,I3,M1,E6,P7);(A2,I3,M1,E6,P8);(A2,I3,M1,E6,P9);(A2,I3,M1,E6,P10);(A2,I3,M1,E6,P11);(A2,I3,M1,E6,P12);(A2,I3,M1,E6,P13);(A2,I3,M1,E6,P14);(A2,I3,M1,E6,P15);(A2,I3,M1,E6,P16);(A2,I3,M1,E6,P17);(A2,I3,M1,E6,P18);(A2,I3,M1,E6,P19);(A2,I3,M1,E6,P20);(A2,I3,M1,E7,P1);(A2,I3,M1,E7,P2);(A2,I3,M1,E7,P3);(A2,I3,M1,E7,P4);(A2,I3,M1,E7,P5);(A2,I3,M1,E7,P6);(A2,I3,M1,E7,P7);(A2,I3,M1,E7,P8);(A2,I3,M1,E7,P9);(A2,I3,M1,E7,P10);(A2,I3,M1,E7,P11);(A2,I3,M1,E7,P12);(A2,I3,M1,E7,P13);(A2,I3,M1,E7,P14);(A2,I3,M1,E7,P15);(A2,I3,M1,E7,P16);(A2,I3,M1,E7,P17);(A2,I3,M1,E7,P18);(A2,I3,M1,E7,P19);(A2,I3,M1,E7,P20);(A2,I3,M1,E8,P1);(A2,I3,M1,E8,P2);(A2,I3,M1,E8,P3);(A2,I3,M1,E8,P4);(A2,I3,M1,E8,P5);(A2,I3,M1,E8,P6);(A2,I3,M1,E8,P7);(A2,I3,M1,E8,P8);(A2,I3,M1,E8,P9);(A2,I3,M1,E8,P10);(A2,I3,M1,E8,P11);(A2,I3,M1,E8,P12);(A2,I3,M1,E8,P13);(A2,I3,M1,E8,P14);(A2,I3,M1,E8,P15);(A2,I3,M1,E8,P16);(A2,I3,M1,E8,P17);(A2,I3,M1,E8,P18);(A2,I3,M1,E8,P19);(A2,I3,M1,E8,P20);(A2,I3,M1,E9,P1);(A2,I3,M1,E9,P2);(A2,I3,M1,E9,P3);(A2,I3,M1,E9,P4);(A2,I3,M1,E9,P5);(A2,I3,M1,E9,P6);(A2,I3,M1,E9,P7);(A 2,I3,M1,E9,P8);(A2,I3,M1,E9,P9);(A2,I3,M1,E9,P10);(A2,I3,M1,E9,P11);(A2,I3,M1,E9,P12);(A2,I3,M1,E9,P13);(A2,I3,M1,E9,P14);(A2,I3,M1,E9,P15);(A2,I3,M1,E9,P16);(A2,I3,M1,E9,P17);(A2,I3,M1,E9,P18);(A2,I3,M1,E9,P19);(A2,I3,M1,E9,P20);(A2,I3,M2,E1,P1);(A2,I3,M2,E1,P2);(A2,I3,M2,E1,P3);(A2,I3,M2,E1,P4);(A2,I3,M2,E1,P5);(A2,I3,M2,E1,P6);(A2,I3,M2,E1,P7);(A2,I3,M2,E1,P8);(A2,I3,M2,E1,P9);(A2,I3,M2,E1,P10);(A2,I3,M2,E1,P11);(A2,I3,M2,E1,P12);(A2,I3,M2,E1,P13);(A2,I3,M2,E1,P14);(A2,I3,M2,E1,P15);(A2,I3,M2,E1,P16);(A2,I3,M2,E1,P17);(A2,I3,M2,E1,P18);(A2,I3,M2,E1,P19);(A2,I3,M2,E1,P20);(A2,I3,M2,E2,P1);(A2,I3,M2,E2,P2);(A2,I3,M2,E2,P3);(A2,I3,M2,E2,P4);(A2,I3,M2,E2,P5);(A2,I3,M2,E2,P6);(A2,I3,M2,E2,P7);(A2,I3,M2,E2,P8);(A2,I3,M2,E2,P9);(A2,I3,M2,E2,P10);(A2,I3,M2,E2,P11);(A2,I3,M2,E2,P12);(A2,I3,M2,E2,P13);(A2,I3,M2,E2,P14);(A2,I3,M2,E2,P15);(A2,I3,M2,E2,P16);(A2,I3,M2,E2,P17);(A2,I3,M2,E2,P18);(A2,I3,M2,E2,P19);(A2,I3,M2,E2,P20);(A2,I3,M2,E3,P1);(A2,I3,M2,E3,P2);(A2,I3,M2,E3,P3);(A2,I3,M2,E3,P4);(A2,I3,M2,E3,P5);(A2,I3,M2,E3,P6);(A2,I3,M2,E3,P7);(A2,I3,M2,E3,P8);(A2,I3,M2,E3,P9);(A2,I3,M2,E3,P10);(A2,I3,M2,E3,P11);(A2,I3,M2,E3,P12);(A2,I3,M2,E3,P13);(A2,I3,M2,E3,P14);(A2,I3,M2,E3,P15);(A2,I3,M2,E3,P16);(A2,I3,M2,E3,P17);(A2,I3,M2,E3,P18);(A2,I3,M2,E3,P19);(A2,I3,M2,E3,P20);(A2,I3,M2,E4,P1);(A2,I3,M2,E4,P2);(A2,I3,M2,E4,P3);(A2,I3,M2,E4,P4);(A2,I3,M2,E4,P5);(A2,I3,M2,E4,P6);(A2,I3,M2,E4,P7);(A2,I3,M2,E4,P8);(A2,I3,M2,E4,P9);(A2,I3,M2,E4,P10);(A2,I3,M2,E4,P11);(A2,I3,M2,E4,P12);(A2,I3,M2,E4,P13);(A2,I3,M2,E4,P14);(A2,I3,M2,E4,P15);(A2,I3,M2,E4,P16);(A2,I3,M2,E4,P17);(A2,I3,M2,E4,P18);(A2,I3,M2,E4,P19);(A2,I3,M2,E4,P20);(A2,I3,M2,E5,P1);(A2,I3,M2,E5,P2);(A2,I3,M2,E5,P3);(A2,I3,M2,E5,P4);(A2,I3,M2,E5,P5);(A2,I3,M2,E5,P6);(A2,I3,M2,E5,P7);(A2,I3,M2,E5,P8);(A2,I3,M2,E5,P9);(A2,I3,M2,E5,P10);(A2,I3,M2,E5,P11);(A2,I3,M2,E5,P12);(A2,I3,M2,E5,P13);(A2,I3,M2,E5,P14);(A2,I3,M2,E5,P15);(A2,I3,M2,E5,P16);(A2,I3,M2,E5,P17);(A2,I3,M2,E5,P18);(A2,I3,M2,E5,P19);(A2,I3,M2,E5,P20);(A2,I3,M2,E6,P1);(A2,I3,M2,E6,P2);(A2,I3,M2,E6,P3);(A2,I3,M2,E6,P4);(A2,I3,M2,E6,P5);(A2,I3,M2,E6,P6);(A2,I3,M2,E6,P7);(A2,I3,M2,E6,P8);(A2,I3,M2,E6,P9);(A2,I3,M2,E6,P10);(A2,I3,M2,E6,P11);(A2,I3,M2,E6,P12);(A2,I3,M2,E6,P13);(A2,I3,M2,E6,P14);(A2,I3,M2,E6,P15);(A2,I3,M2,E6,P16);(A2,I3,M2,E6,P17);(A2,I3,M2,E6,P18);(A2,I3,M2,E6,P19);(A2,I3,M2,E6,P20);(A2,I3,M2,E7,P1);(A2,I3,M2,E7,P2);(A2,I3,M2,E7,P3);(A2,I3,M2,E7,P4);(A2,I3,M2,E7,P5);(A2,I3,M2,E7,P6);(A2,I3,M2,E7,P7);(A2,I3,M2,E7,P8);(A2,I3,M2,E7,P9);(A2,I3,M2,E7,P10);(A2,I3,M2,E7,P11);(A2,I3,M2,E7,P12);(A2,I3,M2,E7,P13);(A2,I3,M2,E7,P14);(A2,I3,M2,E7,P15);(A2,I3,M2,E7,P16);(A2,I3,M2,E7,P17);(A2,I3,M2,E7,P18);(A2,I3,M2,E7,P19);(A2,I3,M2,E7,P20);(A2,I3,M2,E8,P1);(A2,I3,M2,E8,P2);(A2,I3,M2,E8,P3);(A2,I3,M2,E8,P4);(A2,I3,M2,E8,P5);(A2,I3,M2,E8,P6);(A2,I3,M2,E8,P7);(A2,I3,M2,E8,P8);(A2,I3,M2,E8,P9);(A2,I3,M2,E8,P10);(A2,I3,M2,E8,P11);(A2,I3,M2,E8,P12);(A2,I3,M2,E8,P13);(A2,I3,M2,E8,P14);(A2,I3,M2,E8,P15);(A2,I3,M2,E8,P16);(A2,I3,M2,E8,P17);(A2,I3,M2,E8,P18);(A2,I3,M2,E8,P19);(A2,I3,M2,E8,P20);(A2,I3,M2,E9,P1);(A2,I3,M2,E9,P2);(A2,I3,M2,E9,P3);(A2,I3,M2,E9,P4);(A2,I3,M2,E9,P5);(A2,I3,M2,E9,P6);(A2,I3,M2,E9,P7);(A2,I3,M2,E9,P8);(A2,I3,M2,E9,P9);(A2,I3,M2,E9,P10);(A2,I3,M2,E9,P11);(A2,I3,M2,E9,P12);(A2,I3,M2,E9,P13);(A2,I3,M2,E9,P14);(A2,I3,M2,E9,P15);(A2,I3,M2,E9,P16);(A2,I3,M2,E9,P17);(A2,I3,M2,E9,P18);(A2,I3,M2,E9,P19);(A2,I3,M2,E9,P20);(A2,I3,M3,E1,P1);(A2,I3,M3,E1,P2);(A2,I3,M3,E1,P3);(A2,I3,M3,E1,P4);(A2,I3,M3,E1,P5);(A2,I3,M3,E1,P6);(A2,I3,M3,E1,P7);(A2,I3,M3,E1,P8);(A2,I3,M3,E1,P9);(A2,I3,M3,E1,P10);(A2,I3,M3,E1,P11);(A2,I3,M3,E1,P12);(A2,I3,M3,E1,P13);(A2,I3,M3,E1,P14);(A2,I3,M3,E1,P15);(A2,I3,M3,E1,P16);(A2,I3,M3,E1,P17);(A2,I3,M3,E1,P18);(A2,I3,M3,E1,P19);(A2,I3,M3,E1,P20);(A2,I3,M3,E2,P1);(A2,I3,M3,E2,P2);(A2,I3,M3,E2,P3);(A2,I3,M3,E2,P4);(A2,I3,M3,E2,P5);(A2,I3,M3,E2,P6);(A2,I3,M3,E2,P7);(A2,I3,M3,E2,P8);(A2,I3,M3,E2,P9);(A2,I3,M3,E2,P10);(A2,I3,M3,E2,P11);(A2,I3,M3,E2,P12);(A2,I3,M3,E2,P13);(A2,I3,M3,E2,P14);(A2,I3,M3,E2,P15);(A2,I3,M3,E2,P16);(A2,I3,M3,E2,P17);(A2,I3,M3,E2,P18);(A2,I3,M3,E2,P19);(A2,I3,M3,E2,P20);(A2,I3,M3,E3,P1);(A2,I3,M3,E3,P2);(A2,I3,M3,E3,P3);(A2,I3,M3,E3,P4);(A2,I3,M3,E3,P5);(A2,I3,M3,E3,P6);(A2,I3,M3,E3,P7);(A2,I3,M3,E3,P8);(A2,I3,M3,E3,P9);(A2,I3,M3,E3,P10);(A2,I3,M3,E3,P11);(A2,I3,M3,E3,P12);(A2,I3,M3,E3,P13);(A2,I3,M3,E3,P14);(A2,I3,M3,E3,P15);(A2,I3,M3,E3,P16);(A2,I3,M3,E3,P17);(A2,I3,M3,E3,P18);(A2,I3,M3,E3,P19);(A2,I3,M3,E3,P20);(A2,I3,M3,E4,P1);(A2,I3,M3,E4,P2);(A2,I3,M3,E4,P3);(A2,I3,M3,E4,P4);(A2,I3,M3,E4,P5);(A2,I3,M3,E4,P6);(A2,I3,M3,E4,P7);(A2,I3,M3,E4,P8);(A2,I3,M3,E4,P9);(A2,I3,M3,E4,P10);(A2,I3,M3,E4,P11);(A2,I3,M3,E4,P12);(A2,I3,M3,E4,P13);(A2,I3,M3,E4,P14);(A2,I3,M3,E4,P15);(A2,I3,M3,E4,P16);(A2,I3,M3,E4,P17);(A2,I3,M3,E4,P18);(A2,I3,M3,E4,P19);(A2,I3,M3,E4,P20);(A2,I3,M3,E5,P1);(A2,I3,M3,E5,P2);(A2,I3,M3,E5,P3);(A2,I3,M3,E5,P4);(A2,I3,M3,E5,P5);(A2,I3,M3,E5,P6);(A2,I3,M3,E5,P7);(A2,I3,M3,E5,P8);(A2,I3,M3,E5,P9);(A2,I3,M3,E5,P10);(A2,I3,M3,E5,P11);(A2,I3,M3,E5,P12);(A2,I3,M3,E5,P13);(A2,I3,M3,E5,P14);(A2,I3,M3,E5,P15);(A2,I3,M3,E5,P16);(A2,I3,M3,E5,P17);(A2,I3,M3,E5,P18);(A2,I3,M3,E5,P19);(A2,I3,M3,E5,P20);(A2,I3,M3,E6,P1);(A2,I3,M3,E6,P2);(A2,I3,M3,E6,P3);(A2,I3,M3,E6,P4);(A2,I3,M3,E6,P5);(A2,I3,M3,E6,P6);(A2,I3,M3,E6,P7);(A2,I3,M3,E6,P8);(A2,I3,M3,E6,P9);(A2,I3,M3,E6,P10);(A2,I3,M3,E6,P11);(A2,I3,M3,E6,P12);(A2,I3,M3,E6,P13);(A2,I3,M3,E6,P14);(A2,I3,M3,E6,P15);(A2,I3,M3,E6,P16);(A2,I3,M3,E6,P17);(A2,I3,M3,E6,P18);(A2,I3,M3,E6,P19);(A2,I3,M3,E6,P20);(A2,I3,M3,E7,P1);(A2,I3,M3,E7,P2);(A2,I3,M3,E7,P3);(A2,I3,M3,E7,P4);(A2,I3,M3,E7,P5);(A2,I3,M3,E7,P6);(A2,I3,M3,E7,P7);(A2,I3,M3,E7,P8);(A2,I3,M3,E7,P9);(A2,I3,M3,E7,P10);(A2,I3,M3,E7,P11);(A2,I3,M3,E7,P12);(A2,I3,M3,E7,P13);(A2,I3,M3,E7,P14);(A2,I3,M3,E7,P15);(A2,I3,M3,E7,P16);(A2,I3,M3,E7,P17);(A2,I3,M3,E7,P18);(A2,I3,M3,E7,P19);(A2,I3,M3,E7,P20);(A2,I3,M3,E8,P1);(A2,I3,M3,E8,P2);(A2,I3,M3,E8,P3);(A2,I3,M3,E8,P4);(A2,I3,M3,E8,P5);(A2,I3,M3,E8,P6);(A2,I3,M3,E8,P7);(A2,I3,M3,E8,P8);(A2,I3,M3,E8,P9);(A2,I3,M3,E8,P10);(A2,I3,M3,E8,P11);(A2,I3,M3,E8,P12);(A2,I3,M3,E8,P13);(A2,I3,M3,E8,P14);(A2,I3,M3,E8,P15);(A2,I3,M3,E8,P16);(A2,I3,M3,E8,P17);(A2,I3,M3,E8,P18);(A2,I3,M3,E8,P19);(A2,I3,M3,E8,P20);(A2,I3,M3,E9,P1);(A2,I3,M3,E9,P2);(A2,I3,M3,E9,P3);(A2,I3,M3,E9,P4);(A2,I3,M3,E9,P5);(A2,I3,M3,E9,P6);(A2,I3,M3,E9,P7);(A2,I3,M3,E9,P8);(A2,I3,M3,E9,P9);(A2,I3,M3,E9,P10);(A2,I3,M3,E9,P11);(A2,I3,M3,E9,P12);(A2,I3,M3,E9,P13);(A2,I3,M3,E9,P14);(A2,I3,M3,E9,P15);(A2,I3,M3,E9,P16);(A2,I3,M3,E9,P17);(A2,I3,M3,E9,P18);(A2,I3,M3,E9,P19);(A2,I3,M3,E9,P20);(A2,I3,M4,E1,P1);(A2,I3,M4,E1,P2);(A2,I3,M4,E1,P3);(A2,I3,M4,E1,P4);(A2,I3,M4,E1,P5);(A2,I3,M4,E1,P6);(A2,I3,M4,E1,P7);(A2,I3,M4,E1,P8);(A2,I3,M4,E1,P9);(A2,I3,M4,E1,P10);(A2,I3,M4,E1,P11);(A2,I3,M4,E1,P12);(A2,I3,M4,E1,P13);(A2,I3,M4,E1,P14);(A2,I3,M4,E1,P15);(A2,I3,M4,E1,P16);(A2,I3,M4,E1,P17);(A2,I3,M4,E1,P18);(A2,I3,M4,E1,P19);(A2,I3,M4,E1,P20);(A2,I3,M4,E2,P1);(A2,I3,M4,E2,P2);(A2,I3,M4,E2,P3);(A2,I3,M4,E2,P4);(A2,I3,M4,E2,P5);(A2,I3,M4,E2,P6);(A2,I3,M4,E2,P7);(A2,I3,M4,E2,P8);(A2,I3,M4,E2,P9);(A2,I3,M4,E2,P10);(A2,I3,M4,E2,P11);(A2,I3,M4,E2,P12);(A2,I3,M4,E2,P13);(A2,I3,M4,E2,P14);(A2,I3,M4,E2,P15);(A2,I3,M4,E2,P16);(A2,I3,M4,E2,P17);(A2,I3,M4,E2,P18);(A2,I3,M4,E2,P19);(A2,I3,M4,E2,P20);(A2,I3,M4,E3,P1);(A2,I3,M4,E3,P2);(A2,I3,M4,E3,P3);(A2,I3,M4,E3,P4);(A2,I3,M4,E3,P5);(A2,I3,M4,E3,P6);(A2,I3,M4,E3,P7);(A2,I3,M4,E3,P8);(A2,I3,M4,E3,P9);(A2,I3,M4,E3,P10);(A2,I3,M4,E3,P11);(A2,I3,M4,E3,P12);(A2,I3,M4,E3,P13);(A2,I3,M4,E3,P14);(A2,I3,M4,E3,P15);(A2,I3,M4,E3,P16);(A2,I3,M4,E3,P17);(A2,I3,M4,E3,P18);(A2,I3,M4,E3,P19);(A2,I3,M4,E3,P20);(A2,I3,M4,E4,P1);(A2,I3,M4,E4,P2);(A2,I3,M4,E4,P3);(A2,I3,M4,E4,P4);(A2,I3,M4,E4,P5);(A2,I3,M4,E4,P6);(A2,I3,M4,E4,P7);(A2,I3,M4,E4,P8);(A2,I3,M4,E4,P9);(A2,I3,M4,E4,P10);(A2,I3,M4,E4,P11);(A2,I3,M4,E4,P12);(A2,I3,M4,E4,P13);(A2,I3,M4,E4,P14);(A2,I3,M4,E4,P15);(A2,I3,M4,E4,P16);(A2,I3,M4,E4,P17);(A2,I3,M4,E4,P18);(A2,I3,M4,E4,P19);(A2,I3,M4,E4,P20);(A2,I3,M4,E5,P1);(A2,I3,M4,E5,P2);(A2,I3,M4,E5,P3);(A2,I3,M4,E5,P4);(A2,I3,M4,E5,P5);(A2,I3,M4,E5,P6);(A2,I3,M4,E5,P7);(A2,I3,M4,E5,P8);(A2,I3,M4,E5,P9);(A2,I3,M4,E5,P10);(A2,I3,M4,E5,P11);(A2,I3,M4,E5,P12);(A2,I3,M4,E5,P13);(A2,I3,M4,E5,P14);(A2,I3,M4,E5,P15);(A2,I3,M4,E5,P16);(A2,I3,M4,E5,P17);(A2,I3,M4,E5,P18);(A2,I3,M4,E5,P19);(A2,I3,M4,E5,P20);(A2,I3,M4,E6,P1);(A2,I3,M4,E6,P2);(A2,I3,M4,E6,P3);(A2,I3,M4,E6,P4);(A2,I3,M4,E6,P5);(A2,I3,M4,E6,P6);(A2,I3,M4,E6,P7);(A2,I3,M4,E6,P8);(A2,I3,M4,E6,P9);(A2,I3,M4,E6,P10);(A2,I3,M4,E6,P11);(A2,I3,M4,E6,P12);(A2,I3,M4,E6,P13);(A2,I3,M4,E6,P14);(A2,I3,M4,E6,P15);(A2,I3,M4,E6,P16);(A2,I3,M4,E6,P17);(A2,I3,M4,E6,P18);(A2,I3,M4,E6,P19);(A2,I3,M4,E6,P20);(A2,I3,M4,E7,P1);(A2,I3,M4,E7,P2);(A2,I3,M4,E7,P3);(A2,I3,M4,E7,P4);(A2,I3,M4,E7,P5);(A2,I3,M4,E7,P6);(A2,I3,M4,E7,P7);(A2,I3,M4,E7,P8);(A2,I3,M4,E7,P9);(A2,I3,M4,E7,P10);(A2,I3,M4,E7,P11);(A2,I3,M4,E7,P12);(A2,I3,M4,E7,P13);(A2,I3,M4,E7,P14);(A2,I3,M4,E7,P15);(A2,I3,M4,E7,P16);(A2,I3,M4,E7,P17);(A2,I3,M4,E7,P18);(A2,I3,M4,E7,P19);(A2,I3,M4,E7,P20);(A2,I3,M4,E8,P1);(A2,I3,M4,E8,P2);(A2,I3,M4,E8,P3);(A2,I3,M4,E8,P4);(A2,I3,M4,E8,P5);(A2,I3,M4,E8,P6);(A2,I3,M4,E8,P7);(A2,I3,M4,E8,P8);(A2,I3,M4,E8,P9);(A2,I3,M4,E8,P10);(A2,I3,M4,E8,P11);(A2,I3,M4,E8,P12);(A2,I3,M4,E8,P13);(A2,I3,M4,E8,P14);(A2,I3,M4,E8,P15);(A2,I3,M4,E8,P16);(A2,I3,M4,E8,P17);(A2,I3,M4,E8,P18);(A2,I3,M4,E8,P19);(A2,I3,M4,E8,P20);(A2,I3,M4,E9,P1);(A2,I3,M4,E9,P2);(A2,I3,M4,E9,P3);(A2,I3,M4,E9,P4);(A2,I3,M4,E9,P5);(A2,I3,M4,E9,P6);(A2,I3,M4,E9,P7);(A2,I3,M4,E9,P8);(A2,I3,M4,E9,P9);(A2,I3,M4,E9,P10);(A2,I3,M4,E9,P11);(A2,I3,M4,E9,P12);(A2,I3,M4,E9,P13);(A2,I3,M4,E9,P14);(A2,I3,M4,E9,P15);(A2,I3,M4,E9,P16);(A2,I3,M4,E9,P17);(A2,I3,M4,E9,P18);(A2,I3,M4,E9,P19);(A2,I3,M4,E9,P20);(A2,I3,M5,E1,P1);(A2,I3,M5,E1,P2);(A2,I3,M5,E1,P3);(A2,I3,M5,E1,P4);(A2,I3,M5,E1,P5);(A2,I3,M5,E1,P6);(A2,I3,M5,E1,P7);(A2,I3,M5,E1,P8);(A2,I3,M5,E1,P9);(A2,I3,M5,E1,P10);(A2,I3,M5,E1,P11);(A2,I3,M5,E1,P12);(A2,I3,M5,E1,P13);(A2,I3,M5,E1,P14);(A2,I3,M5,E1,P15);(A2,I3,M5,E1,P16);(A2,I3,M5,E1,P17);(A2,I3,M5,E1,P18);(A2,I3,M5,E1,P19);(A2,I3,M5,E1,P20);(A2,I3,M5,E2,P1);(A2,I3,M5,E2,P2);(A2,I3,M5,E2,P3);(A2,I3,M5,E2,P4);(A2,I3,M5,E2,P5);(A2,I3,M5,E2,P6);(A2,I3,M5,E2,P7);(A2,I3,M5,E2,P8);(A2,I3,M5,E2,P9);(A2,I3,M5,E2,P10);(A2,I3,M5,E2,P11);(A2,I3,M5,E2,P12);(A2,I3,M5,E2,P13);(A2,I3,M5,E2,P14);(A2,I3,M5,E2,P15);(A2,I3,M5,E2,P16);(A2,I3,M5,E2,P17);(A2,I3,M5,E2,P18);(A2,I3,M5,E2,P19);(A2,I3,M5,E2,P20);(A2,I3,M5,E3,P1);(A2,I3,M5,E3,P2);(A2,I3,M5,E3,P3);(A2,I3,M5,E3,P4);(A2,I3,M5,E3,P5);(A2,I3,M5,E3,P6);(A2,I3,M5,E3,P7);(A2,I3,M5,E3,P8);(A2,I3,M5,E3,P9);(A2,I3,M5,E3,P10);(A2,I3,M5,E3,P11);(A2,I3,M5,E3,P12);(A2,I3,M5,E3,P13);(A2,I3,M5,E3,P14);(A2,I3,M5,E3,P15);(A2,I3,M5,E3,P16);(A2,I3,M5,E3,P17);(A2,I3,M5,E3,P18);(A2,I3,M5,E3,P19);(A2,I3,M5,E3,P20);(A2,I3,M5,E4,P1);(A2,I3,M5,E4,P2);(A2,I3,M5,E4,P3);(A2,I3,M5,E4,P4);(A2,I3,M5,E4,P5);(A2,I3,M5,E4,P6);(A2,I3,M5,E4,P7);(A2,I3,M5,E4,P8);(A2,I3,M5,E4,P9);(A2,I3,M5,E4,P10);(A2,I3,M5,E4,P11);(A2,I3,M5,E4,P12);(A2,I3,M5,E4,P13);(A2,I3,M5,E4,P14);(A2,I3,M5,E4,P15);(A2,I3,M5,E4,P16);(A2,I3,M5,E4,P17);(A2,I3,M5,E4,P18);(A2,I3,M5,E4,P19);(A2,I3,M5,E4,P20);(A2,I3,M5,E5,P1);(A2,I3,M5,E5,P2);(A2,I3,M5,E5,P3);(A2,I3,M5,E5,P4);(A2,I3,M5,E5,P5);(A2,I3,M5,E5,P6);(A2,I3,M5,E5,P7);(A2,I3,M5,E5,P8);(A2,I3,M5,E5,P9);(A2,I3,M5,E5,P10);(A2,I3,M5,E5,P11);(A2,I3,M5,E5,P12);(A2,I3,M5,E5,P13);(A2,I3,M5,E5,P14);(A2,I3,M5,E5,P15);(A2,I3,M5,E5,P16);(A2,I3,M5,E5,P17);(A2,I3,M5,E5,P18);(A2,I3,M5,E5,P19);(A2,I3,M5,E5,P20);(A2,I3,M5,E6,P1);(A2,I3,M5,E6,P2);(A2,I3,M5,E6,P3);(A2,I3,M5,E6,P4);(A2,I3,M5,E6,P5);(A2,I3,M5,E6,P6);(A2,I3,M5,E6,P7);(A2,I3,M5,E6,P8);(A2,I3,M5,E6,P9);(A2,I3,M5,E6,P10);(A2,I3,M5,E6,P11);(A2,I3,M5,E6,P12);(A2,I3,M5,E6,P13);(A2,I3,M5,E6,P14);(A2,I3,M5,E6,P15);(A2,I3,M5,E6,P16);(A2,I3,M5,E6,P17);(A2,I3,M5,E6,P18);(A2,I3,M5,E6,P19);(A2,I3,M5,E6,P20);(A2,I3,M5,E7,P1);(A2,I3,M5,E7,P2);(A2,I3,M5,E7,P3);(A2,I3,M5,E7,P4);(A2,I3,M5,E7,P5);(A2,I3,M5,E7,P6);(A2,I3,M5,E7,P7);(A2,I3,M5,E7,P8);(A2,I3,M5,E7,P9);(A2,I3,M5,E7,P10);(A2,I3,M5,E7,P11);(A2,I3,M5,E7,P12);(A2,I3,M5,E7,P13);(A2,I3,M5,E7,P14);(A2,I3,M5,E7,P15);(A2,I3,M5,E7,P16);(A2,I3,M5,E7,P17);(A2,I3,M5,E7,P18);(A2,I3,M5,E7,P19);(A2,I3,M5,E7,P20);(A2,I3,M5,E8,P1);(A2,I3,M5,E8,P2);(A2,I3,M5,E8,P3);(A2,I3,M5,E8,P4);(A2,I3,M5,E8,P5);(A2,I3,M5,E8,P6);(A2,I3,M5,E8,P7);(A2,I3,M5,E8,P8);(A2,I3,M5,E8,P9);(A2,I3,M5,E8,P10);(A2,I3,M5,E8,P11);(A2,I3,M5,E8,P12);(A2,I3,M5,E8,P13);(A2,I3,M5,E8,P14);(A2,I3,M5,E8,P15);(A2,I3,M5,E8,P16);(A2,I3,M5,E8,P17);(A2,I3,M5,E8,P18);(A2,I3,M5,E8,P19);(A2,I3,M5,E8,P20);(A2,I3,M5,E9,P1);(A2,I3,M5,E9,P2);(A2,I3,M5,E9,P3);(A2,I3,M5,E9,P4);(A2,I3,M5,E9,P5);(A2,I3,M5,E9,P6);(A2,I3,M5,E9,P7);(A2,I3,M5,E9,P8);(A2,I3,M5,E9,P9);(A2,I3,M5,E9,P10);(A2,I3,M5,E9,P11);(A2,I3,M5,E9,P12);(A2,I3,M5,E9,P13);(A2,I3,M5,E9,P14);(A2,I3,M5,E9,P15);(A2,I3,M5,E9,P16);(A2,I3,M5,E9,P17);(A2,I3,M5,E9,P18);(A2,I3,M5,E9,P19);(A2,I3,M5,E9,P20);(A2,I3,M6,E1,P1);(A2,I3,M6,E1,P2);(A2,I3,M6,E1,P3);(A2,I3,M6,E1,P4);(A2,I3,M6,E1,P5);(A2,I3,M6,E1,P6);(A2,I3,M6,E1,P7);(A2,I3,M6,E1,P8);(A2,I3,M6,E1,P9);(A2,I3,M6,E1,P10);(A2,I3,M6,E1,P11);(A2,I3,M6,E1,P12);(A2,I3,M6,E1,P13);(A2,I3,M6,E1,P14);(A2,I3,M6,E1,P15);(A2,I3,M6,E1,P16);(A2,I3,M6,E1,P17);(A2,I3,M6,E1,P18);(A2,I3,M6,E1,P19);(A2,I3,M6,E1,P20);(A2,I3,M6,E2,P1);(A2,I3,M6,E2,P2);(A2,I3,M6,E2,P3);(A2,I3,M6,E2,P4);(A2,I3,M6,E2,P5);(A2,I3,M6,E2,P6);(A2,I3,M6,E2,P7);(A2,I3,M6,E2,P8);(A2,I3,M6,E2,P9);(A2,I3,M6,E2,P10);(A2,I3,M6,E2,P11);(A2,I3,M6,E2,P12);(A2,I3,M6,E2,P13);(A2,I3,M6,E2,P14);(A2,I3,M6,E2,P15);(A2,I3,M6,E2,P16);(A2,I3,M6,E2,P17);(A2,I3,M6,E2,P18);(A2,I3,M6,E2,P19);(A2,I3,M6,E2,P20);(A2,I3,M6,E3,P1);(A2,I3,M6,E3,P2);(A2,I3,M6,E3,P3);(A2,I3,M6,E3,P4);(A2,I3,M6,E3,P5);(A2,I3,M6,E3,P6);(A2,I3,M6,E3,P7);(A2,I3,M6,E3,P8);(A2,I3,M6,E3,P9);(A2,I3,M6,E3,P10);(A2,I3,M6,E3,P11);(A2,I3,M6,E3,P12);(A2,I3,M6,E3,P13);(A2,I3,M6,E3,P14);(A2,I3,M6,E3,P15);(A2,I3,M6,E3,P16);(A2,I3,M6,E3,P17);(A2,I3,M6,E3,P18);(A2,I3,M6,E3,P19);(A2,I3,M6,E3,P20);(A2,I3,M6,E4,P1);(A2,I3,M6,E4,P2);(A2,I3,M6,E4,P3);(A2,I3,M6,E4,P4);(A2,I3,M6,E4,P5);(A2,I3,M6,E4,P6);(A2,I3,M6,E4,P7);(A2,I3,M6,E4,P8);(A2,I3,M6,E4,P9);(A2,I3,M6,E4,P10);(A2,I3,M6,E4,P11);(A2,I3,M6,E4,P12);(A2,I3,M6,E4,P13);(A2,I3,M6,E4,P14);(A2,I3,M6,E4,P15);(A2,I3,M6,E4,P16);(A2,I3,M6,E4,P17);(A2,I3,M6,E4,P18);(A2,I3,M6,E4,P19);(A2,I3,M6,E4,P20);(A2,I3,M6,E5,P1);(A2,I3,M6,E5,P2);(A2,I3,M6,E5,P3);(A2,I3,M6,E5,P4);(A2,I3,M6,E5,P5);(A2,I3,M6,E5,P6);(A2,I3,M6,E5,P7);(A2,I3,M6,E5,P8);(A2,I3,M6,E5,P9);(A2,I3,M6,E5,P10);(A2,I3,M6,E5,P11);(A2,I3,M6,E5,P12);(A2,I3,M6,E5,P13);(A2,I3,M6,E5,P14);(A2,I3,M6,E5,P15);(A2,I3,M6,E5,P16);(A2,I3,M6,E5,P17);(A2,I3,M6,E5,P18);(A2,I3,M6,E5,P19);(A2,I3,M6,E5,P20);(A2,I3,M6,E6,P1);(A2,I3,M6,E6,P2);(A2,I3,M6,E6,P3);(A2,I3,M6,E6,P4);(A2,I3,M6,E6,P5);(A2,I3,M6,E6,P6);(A2,I3,M6,E6,P7);(A2,I3,M6,E6,P8);(A2,I3,M6,E6,P9);(A2,I3,M6,E6,P10);(A2,I3,M6,E6,P11);(A2,I3,M6,E6,P12);(A2,I3,M6,E6,P13);(A2,I3,M6,E6,P14);(A2,I3,M6,E6,P15);(A2,I3,M6,E6,P16);(A2,I3,M6,E6,P17);(A2,I3,M6,E6,P18);(A2,I3,M6,E6,P19);(A2,I3,M6,E6,P20);(A2,I3,M6,E7,P1);(A2,I3,M6,E7,P2);(A2,I3,M6,E7,P3);(A2,I3,M6,E7,P4);(A2,I3,M6,E7,P5);(A2,I3,M6,E7,P6);(A2,I3,M6,E7,P7);(A2,I3,M6,E7,P8);(A2,I3,M6,E7,P9);(A2,I3,M6,E7,P10);(A2,I3,M6,E7,P11);(A2,I3,M6,E7,P12);(A2,I3,M6,E7,P13);(A2,I3,M6,E7,P14);(A2,I3,M6,E7,P15);(A2,I3,M6,E7,P16);(A2,I3,M6,E7,P17);(A2,I3,M6,E7,P18);(A2,I3,M6,E7,P19);(A2,I3,M6,E7,P20);(A2,I3,M6,E8,P1);(A2,I3,M6,E8,P2);(A2,I3,M6,E8,P3);(A2,I3,M6,E8,P4);(A2,I3,M6,E8,P5);(A2,I3,M6,E8,P6);(A2,I3,M6,E8,P7);(A2,I3,M6,E8,P8);(A2,I3,M6,E8,P9);(A2,I3,M6,E8,P10);(A2,I3,M6,E8,P11);(A2,I3,M6,E8,P12);(A2,I3,M6,E8,P13);(A2,I3,M6,E8,P14);(A2,I3,M6,E8,P15);(A2,I3,M6,E8,P16);(A2,I3,M6,E8,P17);(A2,I3,M6,E8,P18);(A2,I3,M6,E8,P19);(A2,I3,M6,E8,P20);(A2,I3,M6,E9,P1);(A2,I3,M6,E9,P2);(A2,I3,M6,E9,P3);(A2,I3,M6,E9,P4);(A2,I3,M6,E9,P5);(A2,I3,M6,E9,P6);(A2,I3,M6,E9,P7);(A2,I3,M6,E9,P8);(A2,I3,M6,E9,P9);(A2,I3,M6,E9,P10);(A2,I3,M6,E9,P11);(A2,I3,M6,E9,P12);(A2,I3,M6,E9,P13);(A2,I3,M6,E9,P14);(A2,I3,M6,E9,P15);(A2,I3,M6,E9,P16);(A2,I3,M6,E9,P17);(A2,I3,M6,E9,P18);(A2,I3,M6,E9,P19);(A2,I3,M6,E9,P20);(A2,I4,M1,E1,P1);(A2,I4,M1,E1,P2);(A2,I4,M1,E1,P3);(A2,I4,M1,E1,P4);(A2,I4,M1,E1,P5);(A2,I4,M1,E1,P6);(A2,I4,M1,E1,P7);(A2,I4,M1,E1,P8);(A2,I4,M1,E1,P9);(A2,I4,M1,E1,P10);(A2,I4,M1,E1,P11);(A2,I4,M1,E1,P12);(A2,I4,M1,E1,P13);(A2,I4,M1,E1,P14);(A2,I4,M1,E1,P15);(A2,I4,M1,E1,P16);(A2,I4,M1,E1,P17);(A2,I4,M1,E1,P18);(A2,I4,M1,E1,P19);(A2,I4,M1,E1,P20);(A2,I4,M1,E2,P1);(A2,I4,M1,E2,P2);(A2,I4,M1,E2,P3);(A2,I4,M1,E2,P4);(A2,I4,M1,E2,P5);(A2,I4,M1,E2,P6);(A2,I4,M1,E2,P7);(A2,I4,M1,E2,P8);(A2,I4,M1,E2,P9);(A2,I4,M1,E2,P10);(A2,I4,M1,E2,P11);(A2,I4,M1,E2,P12);(A2,I4,M1,E2,P13);(A2,I4,M1,E2,P14);(A2,I4,M1,E2,P15);(A2,I4,M1,E2,P16);(A2,I4,M1,E2,P17);(A2,I4,M1,E2,P18);(A2,I4,M1,E2,P19);(A2,I4,M1,E2,P20);(A2,I4,M1,E3,P1);(A2,I4,M1,E3,P2);(A2,I4,M1,E3,P3);(A2,I4,M1,E3,P4);(A2,I4,M1,E3,P5);(A2,I4,M1,E3,P6);(A2,I4,M1,E3,P7);(A2,I4,M1,E3,P8);(A2,I4,M1,E3,P9);(A2,I4,M1,E3,P10);(A2,I4,M1,E3,P11);(A2,I4,M1,E3,P12);(A2,I4,M1,E3,P13);(A2,I4,M1,E3,P14);(A2,I4,M1,E3,P15);(A2,I4,M1,E3,P16);(A2,I4,M1,E3,P17);(A2,I4,M1,E3,P18);(A2,I4,M1,E3,P19);(A2,I4,M1,E3,P20);(A2,I4,M1,E4,P1);(A2,I4,M1,E4,P2);(A2,I4,M1,E4,P3);(A2,I4,M1,E4,P4);(A2,I4,M1,E4,P5);(A2,I4,M1,E4,P6);(A2,I4,M1,E4,P7);(A2,I4,M1,E4,P8);(A2,I4,M1,E4,P9);(A2,I4,M1,E4,P10);(A2,I4,M1,E4,P11);(A2,I4,M1,E4,P12);(A2,I4,M1,E4,P13);(A2,I4,M1,E4,P14);(A2,I4,M1,E4,P15);(A2,I4,M1,E4,P16);(A2,I4,M1,E4,P17);(A2,I4,M1,E4,P18);(A2,I4,M1,E4,P19);(A2,I4,M1,E4,P20);(A2,I4,M1,E5,P1);(A2,I4,M1,E5,P2);(A2,I4,M1,E5,P3);(A2,I4,M1,E5,P4);(A2,I4,M1,E5,P5);(A2,I4,M1,E5,P6);(A2,I4,M1,E5,P7);(A2,I4,M1,E5,P8);(A2,I4,M1,E5,P9);(A2,I4,M1,E5,P10);(A2,I4,M1,E5,P11);(A2,I4,M1,E5,P12);(A2,I4,M1,E5,P13);(A2,I4,M1,E5,P14);(A2,I4,M1,E5,P15);(A2,I4,M1,E5,P16);(A2,I4,M1,E5,P17);(A2,I4,M1,E5,P18);(A2,I4,M1,E5,P19);(A2,I4,M1,E5,P20);(A2,I4,M1,E6,P1);(A2,I4,M1,E6,P2);(A2,I4,M1,E6,P3);(A2,I4,M1,E6,P4);(A2,I4,M1,E6,P5);(A2,I4,M1,E6,P6);(A2,I4,M1,E6,P7);(A2,I4,M1,E6,P8);(A2,I4,M1,E6,P9);(A2,I4,M1,E6,P10);(A2,I4,M1,E6,P11);(A2,I4,M1,E6,P12);(A2,I4,M1,E6,P13);(A2,I4,M1,E6,P14);(A2,I4,M1,E6,P15);(A2,I4,M1,E6,P16);(A2,I4,M1,E6,P17);(A2,I4,M1,E6,P18);(A2,I4,M1,E6,P19);(A2,I4,M1,E6,P20);(A2,I4,M1,E7,P1);(A2,I4,M1,E7,P2);(A2,I4,M1,E7,P3);(A2,I4,M1,E7,P4);(A2,I4,M1,E7,P5);(A2,I4,M1,E7,P6);(A2,I4,M1,E7,P7);(A2,I4,M1,E7,P8);(A2,I4,M1,E7,P9);(A2,I4,M1,E7,P10);(A2,I4,M1,E7,P11);(A2,I4,M1,E7,P12);(A2,I4,M1,E7,P13);(A2,I4,M1,E7,P14);(A2,I4,M1,E7,P15);(A2,I4,M1,E7,P16);(A2,I4,M1,E7,P17);(A2,I4,M1,E7,P18);(A2,I4,M1,E7,P19);(A2,I4,M1,E7,P20);(A2,I4,M1,E8,P1);(A2,I4,M1,E8,P2);(A2,I4,M1,E8,P3);(A2,I4,M1,E8,P4);(A2,I4,M1,E8,P5);(A2,I4,M1,E8,P6);(A2,I4,M1,E8,P7);(A2,I4,M1,E8,P8);(A2,I4,M1,E8,P9);(A2,I4,M1,E8,P10);(A2,I4,M1,E8,P11);(A2,I4,M1,E8,P12);(A2,I4,M1,E8,P13);(A2,I4,M1,E8,P14);(A2,I4,M1,E8,P15);(A2,I4,M1,E8,P16);(A2,I4,M1,E8,P17);(A2,I4,M1,E8,P18);(A2,I4,M1,E8,P19);(A2,I4,M1,E8,P20);(A2,I4,M1,E9,P1);(A2,I4,M1,E9,P2);(A2,I4,M1,E9,P3);(A2,I4,M1,E9,P4);(A2,I4,M1,E9,P5);(A2,I4,M1,E9,P6);(A2,I4,M1,E9,P7);(A2,I4,M1,E9,P8);(A2,I4,M1,E9,P9);(A2,I4,M1,E9,P10);(A2,I4,M1,E9,P11);(A2,I4,M1,E9,P12);(A2,I4,M1,E9,P13);(A2,I4,M1,E9,P14);(A2,I4,M1,E9,P15);(A2,I4,M1,E9,P16);(A2,I4,M1,E9,P17);(A2,I4,M1,E9,P18);(A2,I4,M1,E9,P19);(A2,I4,M1,E9,P20);(A2,I4,M2,E1,P1);(A2,I4,M2,E1,P2);(A2,I4,M2,E1,P3);(A2,I4,M2,E1,P4);(A2,I4,M2,E1,P5);(A2,I4,M2,E1,P6);(A2,I4,M2,E1,P7);(A2,I4,M2,E1,P8);(A2,I4,M2,E1,P9);(A2,I4,M2,E1,P10);(A2,I4,M2,E1,P11);(A2,I4,M2,E1,P12);(A2,I4,M2,E1,P13);(A2,I4,M2,E1,P14);(A2,I4,M2,E1,P15);(A2,I4,M2,E1,P16);(A2,I4,M2,E1,P17);(A2,I4,M2,E1,P18);(A2,I4,M2,E1,P19);(A2,I4,M2,E1,P20);(A2,I4,M2,E2,P1);(A2,I4,M2,E2,P2);(A2,I4,M2,E2,P3);(A2,I4,M2,E2,P4);(A2,I4,M2,E2,P5);(A2,I4,M2,E2,P6);(A2,I4,M2,E2,P7);(A2,I4,M2,E2,P8);(A2,I4,M2,E2,P9);(A2,I4,M2,E2,P10);(A2,I4,M2,E2,P11);(A2,I4,M2,E2,P12);(A2,I4,M2,E2,P13);(A2,I4,M2,E2,P14);(A2,I4,M2,E2,P15);(A2,I4,M2,E2,P16);(A2,I4,M2,E2,P17);(A2,I4,M2,E2,P18);(A2,I4,M2,E2,P19);(A2,I4,M2,E2,P20);(A2,I4,M2,E3,P1);(A2,I4,M2,E3,P2);(A2,I4,M2,E3,P3);(A2,I4,M2,E3,P4);(A2,I4,M2,E3,P5);(A2,I4,M2,E3,P6);(A2,I4,M2,E3,P7);(A2,I4,M2,E3,P8);(A2,I4,M2,E3,P9);(A2,I4,M2,E3,P10);(A2,I4,M2,E3,P11);(A2,I4,M2,E3,P12);(A2,I4,M2,E3,P13);(A2,I4,M2,E3,P14);(A2,I4,M2,E3,P15);(A2,I4,M2,E3,P16);(A2,I4,M2,E3,P17);(A2,I4,M2,E3,P18);(A2,I4,M2,E3,P19);(A2,I4,M2,E3,P20);(A2,I4,M2,E4,P1);(A2,I4,M2,E4,P2);(A2,I4,M2,E4,P3);(A2,I4,M2,E4,P4);(A2,I4,M2,E4,P5);(A2,I4,M2,E4,P6);(A2,I4,M2,E4,P7);(A2,I4,M2,E4,P8);(A2,I4,M2,E4,P9);(A2,I4,M2,E4,P10);(A2,I4,M2,E4,P11);(A2,I4,M2,E4,P12);(A2,I4,M2,E4,P13);(A2,I4,M2,E4,P14);(A2,I4,M2,E4,P15);(A2,I4,M2,E4,P16);(A2,I4,M2,E4,P17);(A2,I4,M2,E4,P18);(A2,I4,M2,E4,P19);(A2,I4,M2,E4,P20);(A2,I4,M2,E5,P1);(A2,I4,M2,E5,P2);(A2,I4,M2,E5,P3);(A2,I4,M2,E5,P4);(A2,I4,M2,E5,P5);(A2,I4,M2,E5,P6);(A2,I4,M2,E5,P7);(A2,I4,M2,E5,P8);(A2,I4,M2,E5,P9);(A2,I4,M2,E5,P10);(A2,I4,M2,E5,P11);(A2,I4,M2,E5,P12);(A2,I4,M2,E5,P13);(A2,I4,M2,E5,P14);(A2,I4,M2,E5,P15);(A2,I4,M2,E5,P16);(A2,I4,M2,E5,P17);(A2,I4,M2,E5,P18);(A2,I4,M2,E5,P19);(A2,I4,M2,E5,P20);(A2,I4,M2,E6,P1);(A2,I4,M2,E6,P2);(A2,I4,M2,E6,P3);(A2,I4,M2,E6,P4);(A2,I4,M2,E6,P5);(A2,I4,M2,E6,P6);(A2,I4,M2,E6,P7);(A2,I4,M2,E6,P8);(A2,I4,M2,E6,P9);(A2,I4,M2,E6,P10);(A2,I4,M2,E6,P11);(A2,I4,M2,E6,P12);(A2,I4,M2,E6,P13);(A2,I4,M2,E6,P14);(A2,I4,M2,E6,P15);(A2,I4,M2,E6,P16);(A2,I4,M2,E6,P17);(A2,I4,M2,E6,P18);(A2,I4,M2,E6,P19);(A2,I4,M2,E6,P20);(A2,I4,M2,E7,P1);(A2,I4,M2,E7,P2);(A2,I4,M2,E7,P3);(A2,I4,M2,E7,P4);(A2,I4,M2,E7,P5);(A2,I4,M2,E7,P6);(A2,I4,M2,E7,P7);(A2,I4,M2,E7,P8);(A2,I4,M2,E7,P9);(A2,I4,M2,E7,P10);(A2,I4,M2,E7,P11);(A2,I4,M2,E7,P12);(A2,I4,M2,E7,P13);(A2,I4,M2,E7,P14);(A2,I4,M2,E7,P15);(A2,I4,M2,E7,P16);(A2,I4,M2,E7,P17);(A2,I4,M2,E7,P18);(A2,I4,M2,E7,P19);(A2,I4,M2,E7,P20);(A2,I4,M2,E8,P1);(A2,I4,M2,E8,P2);(A2,I4,M2,E8,P3);(A2,I4,M2,E8,P4);(A2,I4,M2,E8,P5);(A2,I4,M2,E8,P6);(A2,I4,M2,E8,P7);(A2,I4,M2,E8,P8);(A2,I4,M2,E8,P9);(A2,I4,M2,E8,P10);(A2,I4,M2,E8,P11);(A2,I4,M2,E8,P12);(A2,I4,M2,E8,P13);(A2,I4,M2,E8,P14);(A2,I4,M2,E8,P15);(A2,I4,M2,E8,P16);(A2,I4,M2,E8,P17);(A2,I4,M2,E8,P18);(A2,I4,M2,E8,P19);(A2,I4,M2,E8,P20);(A2,I4,M2,E9,P1);(A2,I4,M2,E9,P2);(A2,I4,M2,E9,P3);(A2,I4,M2,E9,P4);(A2,I4,M2,E9,P5);(A2,I4,M2,E9,P6);(A2,I4,M2,E9,P7);(A2,I4,M2,E9,P8);(A2,I4,M2,E9,P9);(A2,I4,M2,E9,P10);(A2,I4,M2,E9,P11);(A2,I4,M2,E9,P12);(A2,I4,M2,E9,P13);(A2,I4,M2,E9,P14);(A2,I4,M2,E9,P15);(A2,I4,M2,E9,P16);(A2,I4,M2,E9,P17);(A2,I4,M2,E9,P18);(A2,I4,M2,E9,P19);(A2,I4,M2,E9,P20);(A2,I4,M3,E1,P1);(A2,I4,M3,E1,P2);(A2,I4,M3,E1,P3);(A2,I4,M3,E1,P4);(A2,I4,M3,E1,P5);(A2,I4,M3,E1,P6);(A2,I4,M3,E1,P7);(A2,I4,M3,E1,P8);(A2,I4,M3,E1,P9);(A2,I4,M3,E1,P10);(A2,I4,M3,E1,P11);(A2,I4,M3,E1,P12);(A2,I4,M3,E1,P13);(A2,I4,M3,E1,P14);(A2,I4,M3,E1,P15);(A2,I4,M3,E1,P16);(A2,I4,M3,E1,P17);(A2,I4,M3,E1,P18);(A2,I4,M3,E1,P19);(A2,I4,M3,E1,P20);(A2,I4,M3,E2,P1);(A2,I4,M3,E2,P2);(A2,I4,M3,E2,P3);(A2,I4,M3,E2,P4);(A2,I4,M3,E2,P5);(A2,I4,M3,E2,P6);(A2,I4,M3,E2,P7);(A2,I4,M3,E2,P8);(A2,I4,M3,E2,P9);(A2,I4,M3,E2,P10);(A2,I4,M3,E2,P11);(A2,I4,M3,E2,P12);(A2,I4,M3,E2,P13);(A2,I4,M3,E2,P14);(A2,I4,M3,E2,P15);(A2,I4,M3,E2,P16);(A2,I4,M3,E2,P17);(A2,I4,M3,E2,P18);(A2,I4,M3,E2,P19);(A2,I4,M3,E2,P20);(A2,I4,M3,E3,P1);(A2,I4,M3,E3,P2);(A2,I4,M3,E3,P3);(A2,I4,M3,E3,P4);(A2,I4,M3,E3,P5);(A2,I4,M3,E3,P6);(A2,I4,M3,E3,P7);(A2,I4,M3,E3,P8);(A2,I4,M3,E3,P9);(A2,I4,M3,E3,P10);(A2,I4,M3,E3,P11);(A2,I4,M3,E3,P12);(A2,I4,M3,E3,P13);(A2,I4,M3,E3,P14);(A2,I4,M3,E3,P15);(A2,I4,M3,E3,P16);(A2,I4,M3,E3,P17);(A2,I4,M3,E3,P18);(A2,I4,M3,E3,P19);(A2,I4,M3,E3,P20);(A2,I4,M3,E4,P1);(A2,I4,M3,E4,P2);(A2,I4,M3,E4,P3);(A2,I4,M3,E4,P4);(A2,I4,M3,E4,P5);(A2,I4,M3,E4,P6);(A2,I4,M3,E4,P7);(A2,I4,M3,E4,P8);(A2,I4,M3,E4,P9);(A2,I4,M3,E4,P10);(A2,I4,M3,E4,P11);(A2,I4,M3,E4,P12);(A2,I4,M3,E4,P13);(A2,I4,M3,E4,P14);(A2,I4,M3,E4,P15);(A2,I4,M3,E4,P16);(A2,I4,M3,E4,P17);(A2,I4,M3,E4,P18);(A2,I4,M3,E4,P19);(A2,I4,M3,E4,P20);(A2,I4,M3,E5,P1);(A2,I4,M3,E5,P2);(A2,I4,M3,E5,P3);(A2,I4,M3,E5,P4);(A2,I4,M3,E5,P5);(A2,I4,M3,E5,P6);(A2,I4,M3,E5,P7);(A2,I4,M3,E5,P8);(A2,I4,M3,E5,P9);(A2,I4,M3,E5,P10);(A2,I4,M3,E5,P11);(A2,I4,M3,E5,P12);(A2,I4,M3,E5,P13);(A2,I4,M3,E5,P14);(A2,I4,M3,E5,P15);(A2,I4,M3,E5,P16);(A2,I4,M3,E5,P17);(A2,I4,M3,E5,P18);(A2,I4,M3,E5,P19);(A2,I4,M3,E5,P20);(A2,I4,M3,E6,P1);(A2,I4,M3,E6,P2);(A2,I4,M3,E6,P3);(A2,I4,M3,E6,P4);(A2,I4,M3,E6,P5);(A2,I4,M3,E6,P6);(A2,I4,M3,E6,P7);(A2,I4,M3,E6,P8);(A2,I4,M3,E6,P9);(A2,I4,M3,E6,P10);(A2,I4,M3,E6,P11);(A2,I4,M3,E6,P12);(A2,I4,M3,E6,P13);(A2,I4,M3,E6,P14);(A2,I4,M3,E6,P15);(A2,I4,M3,E6,P16);(A2,I4,M3,E6,P17);(A2,I4,M3,E6,P18);(A2,I4,M3,E6,P19);(A2,I4,M3,E6,P20);(A2,I4,M3,E7,P1);(A2,I4,M3,E7,P2);(A2,I4,M3,E7,P3);(A2,I4,M3,E7,P4);(A2,I4,M3,E7,P5);(A2,I4,M3,E7,P6);(A2,I4,M3,E7,P7);(A2,I4,M3,E7,P8);(A2,I4,M3,E7,P9);(A2,I4,M3,E7,P10);(A2,I4,M3,E7,P11);(A2,I4,M3,E7,P12);(A2,I4,M3,E7,P13);(A2,I4,M3,E7,P14);(A2,I4,M3,E7,P15);(A2,I4,M3,E7,P16);(A2,I4,M3,E7,P17);(A2,I4,M3,E7,P18);(A2,I4,M3,E7,P19);(A2,I4,M3,E7,P20);(A2,I4,M3,E8,P1);(A2,I4,M3,E8,P2);(A2,I4,M3,E8,P3);(A2,I4,M3,E8,P4);(A2,I4,M3,E8,P5);(A2,I4,M3,E8,P6);(A2,I4,M3,E8,P7);(A2,I4,M3,E8,P8);(A2,I4,M3,E8,P9);(A2,I4,M3,E8,P10);(A2,I4,M3,E8,P11);(A2,I4,M3,E8,P12);(A2,I4,M3,E8,P13);(A2,I4,M3,E8,P14);(A2,I4,M3,E8,P15);(A2,I4,M3,E8,P16);(A2,I4,M3,E8,P17);(A2,I4,M3,E8,P18);(A2,I4,M3,E8,P19);(A2,I4,M3,E8,P20);(A2,I4,M3,E9,P1);(A2,I4,M3,E9,P2);(A2,I4,M3,E9,P3);(A2,I4,M3,E9,P4);(A2,I4,M3,E9,P5);(A2,I4,M3,E9,P6);(A2,I4,M3,E9,P7);(A2,I4,M3,E9,P8);(A2,I4,M3,E9,P9);(A2,I4,M3,E9,P10);(A2,I4,M3,E9,P11);(A2,I4,M3,E9,P12);(A2,I4,M3,E9,P13);(A2,I4,M3,E9,P14);(A2,I4,M3,E9,P15);(A2,I4,M3,E9,P16);(A2,I4,M3,E9,P17);(A2,I4,M3,E9,P18);(A2,I4,M3,E9,P19);(A2,I4,M3,E9,P20);(A2,I4,M4,E1,P1);(A2,I4,M4,E1,P2);(A2,I4,M4,E1,P3);(A2,I4,M4,E1,P4);(A2,I4,M4,E1,P5);(A2,I4,M4,E1,P6);(A2,I4,M4,E1,P7);(A2,I4,M4,E1,P8);(A2,I4,M4,E1,P9);(A2,I4,M4,E1,P10);(A2,I4,M4,E1,P11);(A2,I4,M4,E1,P12);(A2,I4,M4,E1,P13);(A2,I4,M4,E1,P14);(A2,I4,M4,E1,P15);(A2,I4,M4,E1,P16);(A2,I4,M4,E1,P17);(A2,I4,M4,E1,P18);(A2,I4,M4,E1,P19);(A2,I4,M4,E1,P20);(A2,I4,M4,E2,P1);(A2,I4,M4,E2,P2);(A2,I4,M4,E2,P3);(A2,I4,M4,E2,P4);(A2,I4,M4,E2,P5);(A2,I4,M4,E2,P6);(A2,I4,M4,E2,P7);(A2,I4,M4,E2,P8);(A2,I4,M4,E2,P9);(A2,I4,M4,E2,P10);(A2,I4,M4,E2,P11);(A2,I4,M4,E2,P12);(A2,I4,M4,E2,P13);(A2,I4,M4,E2,P14);(A2,I4,M4,E2,P15);(A2,I4,M4,E2,P16);(A2,I4,M4,E2,P17);(A2,I4,M4,E2,P18);(A2,I4,M4,E2,P19);(A2,I4,M4,E2,P20);(A2,I4,M4,E3,P1);(A2,I4,M4,E3,P2);(A2,I4,M4,E3,P3);(A2,I4,M4,E3,P4);(A2,I4,M4,E3,P5);(A2,I4,M4,E3,P6);(A2,I4,M4,E3,P7);(A2,I4,M4,E3,P8);(A2,I4,M4,E3,P9);(A2,I4,M4,E3,P10);(A2,I4,M4,E3,P11);(A2,I4,M4,E3,P12);(A2,I4,M4,E3,P13);(A2,I4,M4,E3,P14);(A2,I4,M4,E3,P15);(A2,I4,M4,E3,P16);(A2,I4,M4,E3,P17);(A2,I4,M4,E3,P18);(A2,I4,M4,E3,P19);(A2,I4,M4,E3,P20);(A2,I4,M4,E4,P1);(A2,I4,M4,E4,P2);(A2,I4,M4,E4,P3);(A2,I4,M4,E4,P4);(A2,I4,M4,E4,P5);(A2,I4,M4,E4,P6);(A2,I4,M4,E4,P7);(A2,I4,M4,E4,P8);(A2,I4,M4,E4,P9);(A2,I4,M4,E4,P10);(A2,I4,M4,E4,P11);(A2,I4,M4,E4,P12);(A2,I4,M4,E4,P13);(A2,I4,M4,E4,P14);(A2,I4,M4,E4,P15);(A2,I4,M4,E4,P16);(A2,I4,M4,E4,P17);(A2,I4,M4,E4,P18);(A2,I4,M4,E4,P19);(A2,I4,M4,E4,P20);(A2,I4,M4,E5,P1);(A2,I4,M4,E5,P2);(A2,I4,M4,E5,P3);(A2,I4,M4,E5,P4);(A2,I4,M4,E5,P5);(A2,I4,M4,E5,P6);(A2,I4,M4,E5,P7);(A2,I4,M4,E5,P8);(A2,I4,M4,E5,P9);(A2,I4,M4,E5,P10);(A2,I4,M4,E5,P11);(A2,I4,M4,E5,P12);(A2,I4,M4,E5,P13);(A2,I4,M4,E5,P14);(A2,I4,M4,E5,P15);(A2,I4,M4,E5,P16);(A2,I4,M4,E5,P17);(A2,I4,M4,E5,P18);(A2,I4,M4,E5,P19);(A2,I4,M4,E5,P20);(A2,I4,M4,E6,P1);(A2,I4,M4,E6,P2);(A2,I4,M4,E6,P3);(A2,I4,M4,E6,P4);(A2,I4,M4,E6,P5);(A2,I4,M4,E6,P6);(A2,I4,M4,E6,P7);(A2,I4,M4,E6,P8);(A2,I4,M4,E6,P9);(A2,I4,M4,E6,P10);(A2,I4,M4,E6,P11);(A2,I4,M4,E6,P12);(A2,I4,M4,E6,P13);(A2,I4,M4,E6,P14);(A2,I4,M4,E6,P15);(A2,I4,M4,E6,P16);(A2,I4,M4,E6,P17);(A2,I4,M4,E6,P18);(A2,I4,M4,E6,P19);(A2,I4,M4,E6,P20);(A2,I4,M4,E7,P1);(A2,I4,M4,E7,P2);(A2,I4,M4,E7,P3);(A2,I4,M4,E7,P4);(A2,I4,M4,E7,P5);(A2,I4,M4,E7,P6);(A2,I4,M4,E7,P7);(A2,I4,M4,E7,P8);(A2,I4,M4,E7,P9);(A2,I4,M4,E7,P10);(A2,I4,M4,E7,P11);(A2,I4,M4,E7,P12);(A2,I4,M4,E7,P13);(A2,I4,M4,E7,P14);(A2,I4,M4,E7,P15);(A2,I4,M4,E7,P16);(A2,I4,M4,E7,P17);(A2,I4,M4,E7,P18);(A2,I4,M4,E7,P19);(A2,I4,M4,E7,P20);(A2,I4,M4,E8,P1);(A2,I4,M4,E8,P2);(A2,I4,M4,E8,P3);(A2,I4,M4,E8,P4);(A2,I4,M4,E8,P5);(A2,I4,M4,E8,P6);(A2,I4,M4,E8,P7);(A2,I4,M4,E8,P8);(A2,I4,M4,E8,P9);(A2,I4,M4,E8,P10);(A2,I4,M4,E8,P11);(A2,I4,M4,E8,P12);(A2,I4,M4,E8,P13);(A2,I4,M4,E8,P14);(A2,I4,M4,E8,P15);(A2,I4,M4,E8,P16);(A2,I4,M4,E8,P17);(A2,I4,M4,E8,P18);(A2,I4,M4,E8,P19);(A2,I4,M4,E8,P20);(A2,I4,M4,E9,P1);(A2,I4,M4,E9,P2);(A2,I4,M4,E9,P3);(A2,I4,M4,E9,P4);(A2,I4,M4,E9,P5);(A2,I4,M4,E9,P6);(A2,I4,M4,E9,P7);(A2,I4,M4,E9,P8);(A2,I4,M4,E9,P9);(A2,I4,M4,E9,P10);(A2,I4,M4,E9,P11);(A2,I4,M4,E9,P12);(A2,I4,M4,E9,P13);(A2,I4,M4,E9,P14);(A2,I4,M4,E9,P15);(A2,I4,M4,E9,P16);(A2,I4,M4,E9,P17);(A2,I4,M4,E9,P18);(A2,I4,M4,E9,P19);(A2,I4,M4,E9,P20);(A2,I4,M5,E1,P1);(A2,I4,M5,E1,P2);(A2,I4,M5,E1,P3);(A2,I4,M5,E1,P4);(A2,I4,M5,E1,P5);(A2,I4,M5,E1,P6);(A2,I4,M5,E1,P7);(A2,I4,M5,E1,P8);(A2,I4,M5,E1,P9);(A2,I4,M5,E1,P10);(A2,I4,M5,E1,P11);(A2,I4,M5,E1,P12);(A2,I4,M5,E1,P13);(A2,I4,M5,E1,P14);(A2,I4,M5,E1,P15);(A2,I4,M5,E1,P16);(A2,I4,M5,E1,P17);(A2,I4,M5,E1,P18);(A2,I4,M5,E1,P19);(A2,I4,M5,E1,P20);(A2,I4,M5,E2,P1);(A2,I4,M5,E2,P2);(A2,I4,M5,E2,P3);(A2,I4,M5,E2,P4);(A2,I4,M5,E2,P5);(A2,I4,M5,E2,P6);(A2,I4,M5,E2,P7);(A2,I4,M5,E2,P8);(A2,I4,M5,E2,P9);(A2,I4,M5,E2,P10);(A2,I4,M5,E2,P11);(A2,I4,M5,E2,P12);(A2,I4,M5,E2,P13);(A2,I4,M5,E2,P14);(A2,I4,M5,E2,P15);(A2,I4,M5,E2,P16);(A2,I4,M5,E2,P17);(A2,I4,M5,E2,P18);(A2,I4,M5,E2,P19);(A2,I4,M5,E2,P20);(A2,I4,M5,E3,P1);(A2,I4,M5,E3,P2);(A2,I4,M5,E3,P3);(A2,I4,M5,E3,P4);(A2,I4,M5,E3,P5);(A2,I4,M5,E3,P6);(A2,I4,M5,E3,P7);(A2,I4,M5,E3,P8);(A2,I4,M5,E3,P9);(A2,I4,M5,E3,P10);(A2,I4,M5,E3,P11);(A2,I4,M5,E3,P12);(A2,I4,M5,E3,P13);(A2,I4,M5,E3,P14);(A2,I4,M5,E3,P15);(A2,I4,M5,E3,P16);(A2,I4,M5,E3,P17);(A2,I4,M5,E3,P18);(A2,I4,M5,E3,P19);(A2,I4,M5,E3,P20);(A2,I4,M5,E4,P1);(A2,I4,M5,E4,P2);(A2,I4,M5,E4,P3);(A2,I4,M5,E4,P4);(A2,I4,M5,E4,P5);(A2,I4,M5,E4,P6);(A2,I4,M5,E4,P7);(A2,I4,M5,E4,P8);(A2,I4,M5,E4,P9);(A2,I4,M5,E4,P10);(A2,I4,M5,E4,P11);(A2,I4,M5,E4,P12);(A2,I4,M5,E4,P13);(A2,I4,M5,E4,P14);(A2,I4,M5,E4,P15);(A2,I4,M5,E4,P16);(A2,I4,M5,E4,P17);(A2,I4,M5,E4,P18);(A2,I4,M5,E4,P19);(A2,I4,M5,E4,P20);(A2,I4,M5,E5,P1);(A2,I4,M5,E5,P2);(A2,I4,M5,E5,P3);(A2,I4,M5,E5,P4);(A2,I4,M5,E5,P5);(A2,I4,M5,E5,P6);(A2,I4,M5,E5,P7);(A2,I4,M5,E5,P8);(A2,I4,M5,E5,P9);(A2,I4,M5,E5,P10);(A2,I4,M5,E5,P11);(A2,I4,M5,E5,P12);(A2,I4,M5,E5,P13);(A2,I4,M5,E5,P14);(A2,I4,M5,E5,P15);(A2,I4,M5,E5,P16);(A2,I4,M5,E5,P17);(A2,I4,M5,E5,P18);(A2,I4,M5,E5,P19);(A2,I4,M5,E5,P20);(A2,I4,M5,E6,P1);(A2,I4,M5,E6,P2);(A2,I4,M5,E6,P3);(A2,I4,M5,E6,P4);(A2,I4,M5,E6,P5);(A2,I4,M5,E6,P6);(A2,I4,M5,E6,P7);(A2,I4,M5,E6,P8);(A2,I4,M5,E6,P9);(A2,I4,M5,E6,P10);(A2,I4,M5,E6,P11);(A2,I4,M5,E6,P12);(A2,I4,M5,E6,P13);(A2,I4,M5,E6,P14);(A2,I4,M5,E6,P15);(A2,I4,M5,E6,P16);(A2,I4,M5,E6,P17);(A2,I4,M5,E6,P18);(A2,I4,M5,E6,P19);(A2,I4,M5,E6,P20);(A2,I4,M5,E7,P1);(A2,I4,M5,E7,P2);(A2,I4,M5,E7,P3);(A2,I4,M5,E7,P4);(A2,I4,M5,E7,P5);(A2,I4,M5,E7,P6);(A2,I4,M5,E7,P7);(A2,I4,M5,E7,P8);(A2,I4,M5,E7,P9);(A2,I4,M5,E7,P10);(A2,I4,M5,E7,P11);(A2,I4,M5,E7,P12);(A2,I4,M5,E7,P13);(A2,I4,M5,E7,P14);(A2,I4,M5,E7,P15);(A2,I4,M5,E7,P16);(A2,I4,M5,E7,P17);(A2,I4,M5,E7,P18);(A2,I4,M5,E7,P19);(A2,I4,M5,E7,P20);(A2,I4,M5,E8,P1);(A2,I4,M5,E8,P2);(A2,I4,M5,E8,P3);(A2,I4,M5,E8,P4);(A2,I4,M5,E8,P5);(A2,I4,M5,E8,P6);(A2,I4,M5,E8,P7);(A2,I4,M5,E8,P8);(A2,I4,M5,E8,P9);(A2,I4,M5,E8,P10);(A2,I4,M5,E8,P11);(A2,I4,M5,E8,P12);(A2,I4,M5,E8,P13);(A2,I4,M5,E8,P14);(A2,I4,M5,E8,P15);(A2,I4,M5,E8,P16);(A2,I4,M5,E8,P17);(A2,I4,M5,E8,P18);(A2,I4,M5,E8,P19);(A2,I4,M5,E8,P20);(A2,I4,M5,E9,P1);(A2,I4,M5,E9,P2);(A2,I4,M5,E9,P3);(A2,I4,M5,E9,P4);(A2,I4,M5,E9,P5);(A2,I4,M5,E9,P6);(A2,I4,M5,E9,P7);(A2,I4,M5,E9,P8);(A2,I4,M5,E9,P9);(A2,I4,M5,E9,P10);(A2,I4,M5,E9,P11);(A2,I4,M5,E9,P12);(A2,I4,M5,E9,P13);(A2,I4,M5,E9,P14);(A2,I4,M5,E9,P15);(A2,I4,M5,E9,P16);(A2,I4,M5,E9,P17);(A2,I4,M5,E9,P18);(A2,I4,M5,E9,P19);(A2,I4,M5,E9,P20);(A2,I4,M6,E1,P1);(A2,I4,M6,E1,P2);(A2,I4,M6,E1,P3);(A2,I4,M6,E1,P4);(A2,I4,M6,E1,P5);(A2,I4,M6,E1,P6);(A2,I4,M6,E1,P7);(A2,I4,M6,E1,P8);(A2,I4,M6,E1,P9);(A2,I4,M6,E1,P10);(A2,I4,M6,E1,P11);(A2,I4,M6,E1,P12);(A2,I4,M6,E1,P13);(A2,I4,M6,E1,P14);(A2,I4,M6,E1,P15);(A2,I4,M6,E1,P16);(A2,I4,M6,E1,P17);(A2,I4,M6,E1,P18);(A2,I4,M6,E1,P19);(A2,I4,M6,E1,P20);(A2,I4,M6,E2,P1);(A2,I4,M6,E2,P2);(A2,I4,M6,E2,P3);(A2,I4,M6,E2,P4);(A2,I4,M6,E2,P5);(A2,I4,M6,E2,P6);(A2,I4,M6,E2,P7);(A2,I4,M6,E2,P8);(A2,I4,M6,E2,P9);(A2,I4,M6,E2,P10);(A2,I4,M6,E2,P11);(A2,I4,M6,E2,P12);(A2,I4,M6,E2,P13);(A2,I4,M6,E2,P14);(A2,I4,M6,E2,P15);(A2,I4,M6,E2,P16);(A2,I4,M6,E2,P17);(A2,I4,M6,E2,P18);(A2,I4,M6,E2,P19);(A2,I4,M6,E2,P20);(A2,I4,M6,E3,P1);(A2,I4,M6,E3,P2);(A2,I4,M6,E3,P3);(A2,I4,M6,E3,P4);(A2,I4,M6,E3,P5);(A2,I4,M6,E3,P6);(A2,I4,M6,E3,P7);(A2,I4,M6,E3,P8);(A2,I4,M6,E3,P9);(A2,I4,M6,E3,P10);(A2,I4,M6,E3,P11);(A2,I4,M6,E3,P12);(A2,I4,M6,E3,P13);(A2,I4,M6,E3,P14);(A2,I4,M6,E3,P15);(A2,I4,M6,E3,P16);(A2,I4,M6,E3,P17);(A2,I4,M6,E3,P18);(A2,I4,M6,E3,P19);(A2,I4,M6,E3,P20);(A2,I4,M6,E4,P1);(A2,I4,M6,E4,P2);(A2,I4,M6,E4,P3);(A2,I4,M6,E4,P4);(A2,I4,M6,E4,P5);(A2,I4,M6,E4,P6);(A2,I4,M6,E4,P7);(A2,I4,M6,E4,P8);(A2,I4,M6,E4,P9);(A2,I4,M6,E4,P10);(A2,I4,M6,E4,P11);(A2,I4,M6,E4,P12);(A2,I4,M6,E4,P13);(A2,I4,M6,E4,P14);(A2,I4,M6,E4,P15);(A2,I4,M6,E4,P16);(A2,I4,M6,E4,P17);(A2,I4,M6,E4,P18);(A2,I4,M6,E4,P19);(A2,I4,M6,E4,P20);(A2,I4,M6,E5,P1);(A2,I4,M6,E5,P2);(A2,I4,M6,E5,P3);(A2,I4,M6,E5,P4);(A2,I4,M6,E5,P5);(A2,I4,M6,E5,P6);(A2,I4,M6,E5,P7);(A2,I4,M6,E5,P8);(A2,I4,M6,E5,P9);(A2,I4,M6,E5,P10);(A2,I4,M6,E5,P11);(A2,I4,M6,E5,P12);(A2,I4,M6,E5,P13);(A2,I4,M6,E5,P14);(A2,I4,M6,E5,P15);(A2,I4,M6,E5,P16);(A2,I4,M6,E5,P17);(A2,I4,M6,E5,P18);(A2,I4,M6,E5,P19);(A2,I4,M6,E5,P20);(A2,I4,M6,E6,P1);(A2,I4,M6,E6,P2);(A2,I4,M6,E6,P3);(A2,I4,M6,E6,P4);(A2,I4,M6,E6,P5);(A2,I4,M6,E6,P6);(A2,I4,M6,E6,P7);(A2,I4,M6,E6,P8);(A2,I4,M6,E6,P9);(A2,I4,M6,E6,P10);(A2,I4,M6,E6,P11);(A2,I4,M6,E6,P12);(A2,I4,M6,E6,P13);(A2,I4,M6,E6,P14);(A2,I4,M6,E6,P15);(A2,I4,M6,E6,P16);(A2,I4,M6,E6,P17);(A2,I4,M6,E6,P18);(A2,I4,M6,E6,P19);(A2,I4,M6,E6,P20);(A2,I4,M6,E7,P1);(A2,I4,M6,E7,P2);(A2,I4,M6,E7,P3);(A2,I4,M6,E7,P4);(A2,I4,M6,E7,P5);(A2,I4,M6,E7,P6);(A2,I4,M6,E7,P7);(A2,I4,M6,E7,P8);(A2,I4,M6,E7,P9);(A2,I4,M6,E7,P10);(A2,I4,M6,E7,P11);(A2,I4,M6,E7,P12);(A2,I4,M6,E7,P13);(A2,I4,M6,E7,P14);(A2,I4,M6,E7,P15);(A2,I4,M6,E7,P16);(A2,I4,M6,E7,P17);(A2,I4,M6,E7,P18);(A2,I4,M6,E7,P19);(A2,I4,M6,E7,P20);(A2,I4,M6,E8,P1);(A2,I4,M6,E8,P2);(A2,I4,M6,E8,P3);(A2,I4,M6,E8,P4);(A2,I4,M6,E8,P5);(A2,I4,M6,E8,P6);(A2,I4,M6,E8,P7);(A2,I4,M6,E8,P8);(A2,I4,M6,E8,P9);(A2,I4,M6,E8,P10);(A2,I4,M6,E8,P11);(A2,I4,M6,E8,P12);(A2,I4,M6,E8,P13);(A2,I4,M6,E8,P14);(A2,I4,M6,E8,P15);(A2,I4,M6,E8,P16);(A2,I4,M6,E8,P17);(A2,I4,M6,E8,P18);(A2,I4,M6,E8,P19);(A2,I4,M6,E8,P20);(A2,I4,M6,E9,P1);(A2,I4,M6,E9,P2);(A2,I4,M6,E9,P3);(A2,I4,M6,E9,P4);(A2,I4,M6,E9,P5);(A2,I4,M6,E9,P6);(A2,I4,M6,E9,P7);(A2,I4,M6,E9,P8);(A2,I4,M6,E9,P9);(A2,I4,M6,E9,P10);(A2,I4,M6,E9,P11);(A2,I4,M6,E9,P12);(A2,I4,M6,E9,P13);(A2,I4,M6,E9,P14);(A2,I4,M6,E9,P15);(A2,I4,M6,E9,P16);(A2,I4,M6,E9,P17);(A2,I4,M6,E9,P18);(A2,I4,M6,E9,P19);(A2,I4,M6,E9,P20);(A2,I5,M1,E1,P1);(A2,I5,M1,E1,P2);(A2,I5,M1,E1,P3);(A2,I5,M1,E1,P4);(A2,I5,M1,E1,P5);(A2,I5,M1,E1,P6);(A2,I5,M1,E1,P7);(A2,I5,M1,E1,P8);(A2,I5,M1,E1,P9);(A2,I5,M1,E1,P10);(A2,I5,M1,E1,P11);(A2,I5,M1,E1,P12);(A2,I5,M1,E1,P13);(A2,I5,M1,E1,P14);(A2,I5,M1,E1,P15);(A2,I5,M1,E1,P16);(A2,I5,M1,E1,P17);(A2,I5,M1,E1,P18);(A2,I5,M1,E1,P19);(A2,I5,M1,E1,P20);(A2,I5,M1,E2,P1);(A2,I5,M1,E2,P2);(A2,I5,M1,E2,P3);(A2,I5,M1,E2,P4);(A2,I5,M1,E2,P5);(A2,I5,M1,E2,P6);(A2,I5,M1,E2,P7);(A2,I5,M1,E2,P8);(A2,I5,M1,E2,P9);(A2,I5,M1,E2,P10);(A2,I5,M1,E2,P11);(A2,I5,M1,E2,P12);(A2,I5,M1,E2,P13);(A2,I5,M1,E2,P14);(A2,I5,M1,E2,P15);(A2,I5,M1,E2,P16);(A2,I5,M1,E2,P17);(A2,I5,M1,E2,P18);(A2,I5,M1,E2,P19);(A2,I5,M1,E2,P20);(A2,I5,M1,E3,P1);(A2,I5,M1,E3,P2);(A2,I5,M1,E3,P3);(A2,I5,M1,E3,P4);(A2,I5,M1,E3,P5);(A2,I5,M1,E3,P6);(A2,I5,M1,E3,P7);(A2,I5,M1,E3,P8);(A2,I5,M1,E3,P9);(A2,I5,M1,E3,P10);(A2,I5,M1,E3,P11);(A2,I5,M1,E3,P12);(A2,I5,M1,E3,P13);(A2,I5,M1,E3,P14);(A2,I5,M1,E3,P15);(A2,I5,M1,E3,P16);(A2,I5,M1 ,E3,P17);(A2,I5,M1,E3,P18);(A2,I5,M1,E3,P19);(A2,I5,M1,E3,P20);(A2,I5,M1,E4,P1);(A2,I5,M1,E4,P2);(A2,I5,M1,E4,P3);(A2,I5,M1,E4,P4);(A2,I5,M1,E4,P5);(A2,I5,M1,E4,P6);(A2,I5,M1,E4,P7);(A2,I5,M1,E4,P8);(A2,I5,M1,E4,P9);(A2,I5,M1,E4,P10);(A2,I5,M1,E4,P11);(A2,I5,M1,E4,P12);(A2,I5,M1,E4,P13);(A2,I5,M1,E4,P14);(A2,I5,M1,E4,P15);(A2,I5,M1,E4,P16);(A2,I5,M1,E4,P17);(A2,I5,M1,E4,P18);(A2,I5,M1,E4,P19);(A2,I5,M1,E4,P20);(A2,I5,M1,E5,P1);(A2,I5,M1,E5,P2);(A2,I5,M1,E5,P3);(A2,I5,M1,E5,P4);(A2,I5,M1,E5,P5);(A2,I5,M1,E5,P6);(A2,I5,M1,E5,P7);(A2,I5,M1,E5,P8);(A2,I5,M1,E5,P9);(A2,I5,M1,E5,P10);(A2,I5,M1,E5,P11);(A2,I5,M1,E5,P12);(A2,I5,M1,E5,P13);(A2,I5,M1,E5,P14);(A2,I5,M1,E5,P15);(A2,I5,M1,E5,P16);(A2,I5,M1,E5,P17);(A2,I5,M1,E5,P18);(A2,I5,M1,E5,P19);(A2,I5,M1,E5,P20);(A2,I5,M1,E6,P1);(A2,I5,M1,E6,P2);(A2,I5,M1,E6,P3);(A2,I5,M1,E6,P4);(A2,I5,M1,E6,P5);(A2,I5,M1,E6,P6);(A2,I5,M1,E6,P7);(A2,I5,M1,E6,P8);(A2,I5,M1,E6,P9);(A2,I5,M1,E6,P10);(A2,I5,M1,E6,P11);(A2,I5,M1,E6,P12);(A2,I5,M1,E6,P13);(A2,I5,M1,E6,P14);(A2,I5,M1,E6,P15);(A2,I5,M1,E6,P16);(A2,I5,M1,E6,P17);(A2,I5,M1,E6,P18);(A2,I5,M1,E6,P19);(A2,I5,M1,E6,P20);(A2,I5,M1,E7,P1);(A2,I5,M1,E7,P2);(A2,I5,M1,E7,P3);(A2,I5,M1,E7,P4);(A2,I5,M1,E7,P5);(A2,I5,M1,E7,P6);(A2,I5,M1,E7,P7);(A2,I5,M1,E7,P8);(A2,I5,M1,E7,P9);(A2,I5,M1,E7,P10);(A2,I5,M1,E7,P11);(A2,I5,M1,E7,P12);(A2,I5,M1,E7,P13);(A2,I5,M1,E7,P14);(A2,I5,M1,E7,P15);(A2,I5,M1,E7,P16);(A2,I5,M1,E7,P17);(A2,I5,M1,E7,P18);(A2,I5,M1,E7,P19);(A2,I5,M1,E7,P20);(A2,I5,M1,E8,P1);(A2,I5,M1,E8,P2);(A2,I5,M1,E8,P3);(A2,I5,M1,E8,P4);(A2,I5,M1,E8,P5);(A2,I5,M1,E8,P6);(A2,I5,M1,E8,P7);(A2,I5,M1,E8,P8);(A2,I5,M1,E8,P9);(A2,I5,M1,E8,P10);(A2,I5,M1,E8,P11);(A2,I5,M1,E8,P12);(A2,I5,M1,E8,P13);(A2,I5,M1,E8,P14);(A2,I5,M1,E8,P15);(A2,I5,M1,E8,P16);(A2,I5,M1,E8,P17);(A2,I5,M1,E8,P18);(A2,I5,M1,E8,P19);(A2,I5,M1,E8,P20);(A2,I5,M1,E9,P1);(A2,I5,M1,E9,P2);(A2,I5,M1,E9,P3);(A2,I5,M1,E9,P4);(A2,I5,M1,E9,P5);(A2,I5,M1,E9,P6);(A2,I5,M1,E9,P7);(A2,I5,M1,E9,P8);(A2,I5,M1,E9,P9);(A2,I5,M1,E9,P10);(A2,I5,M1,E9,P11);(A2,I5,M1,E9,P12);(A2,I5,M1,E9,P13);(A2,I5,M1,E9,P14);(A2,I5,M1,E9,P15);(A2,I5,M1,E9,P16);(A2,I5,M1,E9,P17);(A2,I5,M1,E9,P18);(A2,I5,M1,E9,P19);(A2,I5,M1,E9,P20);(A2,I5,M2,E1,P1);(A2,I5,M2,E1,P2);(A2,I5,M2,E1,P3);(A2,I5,M2,E1,P4);(A2,I5,M2,E1,P5);(A2,I5,M2,E1,P6);(A2,I5,M2,E1,P7);(A2,I5,M2,E1,P8);(A2,I5,M2,E1,P9);(A2,I5,M2,E1,P10);(A2,I5,M2,E1,P11);(A2,I5,M2,E1,P12);(A2,I5,M2,E1,P13);(A2,I5,M2,E1,P14);(A2,I5,M2,E1,P15);(A2,I5,M2,E1,P16);(A2,I5,M2,E1,P17);(A2,I5,M2,E1,P18);(A2,I5,M2,E1,P19);(A2,I5,M2,E1,P20);(A2,I5,M2,E2,P1);(A2,I5,M2,E2,P2);(A2,I5,M2,E2,P3);(A2,I5,M2,E2,P4);(A2,I5,M2,E2,P5);(A2,I5,M2,E2,P6);(A2,I5,M2,E2,P7);(A2,I5,M2,E2,P8);(A2,I5,M2,E2,P9);(A2,I5,M2,E2,P10);(A2,I5,M2,E2,P11);(A2,I5,M2,E2,P12);(A2,I5,M2,E2,P13);(A2,I5,M2,E2,P14);(A2,I5,M2,E2,P15);(A2,I5,M2,E2,P16);(A2,I5,M2,E2,P17);(A2,I5,M2,E2,P18);(A2,I5,M2,E2,P19);(A2,I5,M2,E2,P20);(A2,I5,M2,E3,P1);(A2,I5,M2,E3,P2);(A2,I5,M2,E3,P3);(A2,I5,M2,E3,P4);(A2,I5,M2,E3,P5);(A2,I5,M2,E3,P6);(A2,I5,M2,E3,P7);(A2,I5,M2,E3,P8);(A2,I5,M2,E3,P9);(A2,I5,M2,E3,P10);(A2,I5,M2,E3,P11);(A2,I5,M2,E3,P12);(A2,I5,M2,E3,P13);(A2,I5,M2,E3,P14);(A2,I5,M2,E3,P15);(A2,I5,M2,E3,P16);(A2,I5,M2,E3,P17);(A2,I5,M2,E3,P18);(A2,I5,M2,E3,P19);(A2,I5,M2,E3,P20);(A2,I5,M2,E4,P1);(A2,I5,M2,E4,P2);(A2,I5,M2,E4,P3);(A2,I5,M2,E4,P4);(A2,I5,M2,E4,P5);(A2,I5,M2,E4,P6);(A2,I5,M2,E4,P7);(A2,I5,M2,E4,P8);(A2,I5,M2,E4,P9);(A2,I5,M2,E4,P10);(A2,I5,M2,E4,P11);(A2,I5,M2,E4,P12);(A2,I5,M2,E4,P13);(A2,I5,M2,E4,P14);(A2,I5,M2,E4,P15);(A2,I5,M2,E4,P16);(A2,I5,M2,E4,P17);(A2,I5,M2,E4,P18);(A2,I5,M2,E4,P19);(A2,I5,M2,E4,P20);(A2,I5,M2,E5,P1);(A2,I5,M2,E5,P2);(A2,I5,M2,E5,P3);(A2,I5,M2,E5,P4);(A2,I5,M2,E5,P5);(A2,I5,M2,E5,P6);(A2,I5,M2,E5,P7);(A2,I5,M2,E5,P8);(A2,I5,M2,E5,P9);(A2,I5,M2,E5,P10);(A2,I5,M2,E5,P11);(A2,I5,M2,E5,P12);(A2,I5,M2,E5,P13);(A2,I5,M2,E5,P14);(A2,I5,M2,E5,P15);(A2,I5,M2,E5,P16);(A2,I5,M2,E5,P17);(A2,I5,M2,E5,P18);(A2,I5,M2,E5,P19);(A2,I5,M2,E5,P20);(A2,I5,M2,E6,P1);(A2,I5,M2,E6,P2);(A2,I5,M2,E6,P3);(A2,I5,M2,E6,P4);(A2,I5,M2,E6,P5);(A2,I5,M2,E6,P6);(A2,I5,M2,E6,P7);(A2,I5,M2,E6,P8);(A2,I5,M2,E6,P9);(A2,I5,M2,E6,P10);(A2,I5,M2,E6,P11);(A2,I5,M2,E6,P12);(A2,I5,M2,E6,P13);(A2,I5,M2,E6,P14);(A2,I5,M2,E6,P15);(A2,I5,M2,E6,P16);(A2,I5,M2,E6,P17);(A2,I5,M2,E6,P18);(A2,I5,M2,E6,P19);(A2,I5,M2,E6,P20);(A2,I5,M2,E7,P1);(A2,I5,M2,E7,P2);(A2,I5,M2,E7,P3);(A2,I5,M2,E7,P4);(A2,I5,M2,E7,P5);(A2,I5,M2,E7,P6);(A2,I5,M2,E7,P7);(A2,I5,M2,E7,P8);(A2,I5,M2,E7,P9);(A2,I5,M2,E7,P10);(A2,I5,M2,E7,P11);(A2,I5,M2,E7,P12);(A2,I5,M2,E7,P13);(A2,I5,M2,E7,P14);(A2,I5,M2,E7,P15);(A2,I5,M2,E7,P16);(A2,I5,M2,E7,P17);(A2,I5,M2,E7,P18);(A2,I5,M2,E7,P19);(A2,I5,M2,E7,P20);(A2,I5,M2,E8,P1);(A2,I5,M2,E8,P2);(A2,I5,M2,E8,P3);(A2,I5,M2,E8,P4);(A2,I5,M2,E8,P5);(A2,I5,M2,E8,P6);(A2,I5,M2,E8,P7);(A2,I5,M2,E8,P8);(A2,I5,M2,E8,P9);(A2,I5,M2,E8,P10);(A2,I5,M2,E8,P11);(A2,I5,M2,E8,P12);(A2,I5,M2,E8,P13);(A2,I5,M2,E8,P14);(A2,I5,M2,E8,P15);(A2,I5,M2,E8,P16);(A2,I5,M2,E8,P17);(A2,I5,M2,E8,P18);(A2,I5,M2,E8,P19);(A2,I5,M2,E8,P20);(A2,I5,M2,E9,P1);(A2,I5,M2,E9,P2);(A2,I5,M2,E9,P3);(A2,I5,M2,E9,P4);(A2,I5,M2,E9,P5);(A2,I5,M2,E9,P6);(A2,I5,M2,E9,P7);(A2,I5,M2,E9,P8);(A2,I5,M2,E9,P9);(A2,I5,M2,E9,P10);(A2,I5,M2,E9,P11);(A2,I5,M2,E9,P12);(A2,I5,M2,E9,P13);(A2,I5,M2,E9,P14);(A2,I5,M2,E9,P15);(A2,I5,M2,E9,P16);(A2,I5,M2,E9,P17);(A2,I5,M2,E9,P18);(A2,I5,M2,E9,P19);(A2,I5,M2,E9,P20);(A2,I5,M3,E1,P1);(A2,I5,M3,E1,P2);(A2,I5,M3,E1,P3);(A2,I5,M3,E1,P4);(A2,I5,M3,E1,P5);(A2,I5,M3,E1,P6);(A2,I5,M3,E1,P7);(A2,I5,M3,E1,P8);(A2,I5,M3,E1,P9);(A2,I5,M3,E1,P10);(A2,I5,M3,E1,P11);(A2,I5,M3,E1,P12);(A2,I5,M3,E1,P13);(A2,I5,M3,E1,P14);(A2,I5,M3,E1,P15);(A2,I5,M3,E1,P16);(A2,I5,M3,E1,P17);(A2,I5,M3,E1,P18);(A2,I5,M3,E1,P19);(A2,I5,M3,E1,P20);(A2,I5,M3,E2,P1);(A2,I5,M3,E2,P2);(A2,I5,M3,E2,P3);(A2,I5,M3,E2,P4);(A2,I5,M3,E2,P5);(A2,I5,M3,E2,P6);(A2,I5,M3,E2,P7);(A2,I5,M3,E2,P8);(A2,I5,M3,E2,P9);(A2,I5,M3,E2,P10);(A2,I5,M3,E2,P11);(A2,I5,M3,E2,P12);(A2,I5,M3,E2,P13);(A2,I5,M3,E2,P14);(A2,I5,M3,E2,P15);(A2,I5,M3,E2,P16);(A2,I5,M3,E2,P17);(A2,I5,M3,E2,P18);(A2,I5,M3,E2,P19);(A2,I5,M3,E2,P20);(A2,I5,M3,E3,P1);(A2,I5,M3,E3,P2);(A2,I5,M3,E3,P3);(A2,I5,M3,E3,P4);(A2,I5,M3,E3,P5);(A2,I5,M3,E3,P6);(A2,I5,M3,E3,P7);(A2,I5,M3,E3,P8);(A2,I5,M3,E3,P9);(A2,I5,M3,E3,P10);(A2,I5,M3,E3,P11);(A2,I5,M3,E3,P12);(A2,I5,M3,E3,P13);(A2,I5,M3,E3,P14);(A2,I5,M3,E3,P15);(A2,I5,M3,E3,P16);(A2,I5,M3,E3,P17);(A2,I5,M3,E3,P18);(A2,I5,M3,E3,P19);(A2,I5,M3,E3,P20);(A2,I5,M3,E4,P1);(A2,I5,M3,E4,P2);(A2,I5,M3,E4,P3);(A2,I5,M3,E4,P4);(A2,I5,M3,E4,P5);(A2,I5,M3,E4,P6);(A2,I5,M3,E4,P7);(A2,I5,M3,E4,P8);(A2,I5,M3,E4,P9);(A2,I5,M3,E4,P10);(A2,I5,M3,E4,P11);(A2,I5,M3,E4,P12);(A2,I5,M3,E4,P13);(A2,I5,M3,E4,P14);(A2,I5,M3,E4,P15);(A2,I5,M3,E4,P16);(A2,I5,M3,E4,P17);(A2,I5,M3,E4,P18);(A2,I5,M3,E4,P19);(A2,I5,M3,E4,P20);(A2,I5,M3,E5,P1);(A2,I5,M3,E5,P2);(A2,I5,M3,E5,P3);(A2,I5,M3,E5,P4);(A2,I5,M3,E5,P5);(A2,I5,M3,E5,P6);(A2,I5,M3,E5,P7);(A2,I5,M3,E5,P8);(A2,I5,M3,E5,P9);(A2,I5,M3,E5,P10);(A2,I5,M3,E5,P11);(A2,I5,M3,E5,P12);(A2,I5,M3,E5,P13);(A2,I5,M3,E5,P14);(A2,I5,M3,E5,P15);(A2,I5,M3,E5,P16);(A2,I5,M3,E5,P17);(A2,I5,M3,E5,P18);(A2,I5,M3,E5,P19);(A2,I5,M3,E5,P20);(A2,I5,M3,E6,P1);(A2,I5,M3,E6,P2);(A2,I5,M3,E6,P3);(A2,I5,M3,E6,P4);(A2,I5,M3,E6,P5);(A2,I5,M3,E6,P6);(A2,I5,M3,E6,P7);(A2,I5,M3,E6,P8);(A2,I5,M3,E6,P9);(A2,I5,M3,E6,P10);(A2,I5,M3,E6,P11);(A2,I5,M3,E6,P12);(A2,I5,M3,E6,P13);(A2,I5,M3,E6,P14);(A2,I5,M3,E6,P15);(A2,I5,M3,E6,P16);(A2,I5,M3,E6,P17);(A2,I5,M3,E6,P18);(A2,I5,M3,E6,P19);(A2,I5,M3,E6,P20);(A2,I5,M3,E7,P1);(A2,I5,M3,E7,P2);(A2,I5,M3,E7,P3);(A2,I5,M3,E7,P4);(A2,I5,M3,E7,P5);(A2,I5,M3,E7,P6);(A2,I5,M3,E7,P7);(A2,I5,M3,E7,P8);(A2,I5,M3,E7,P9);(A2,I5,M3,E7,P10);(A2,I5,M3,E7,P11);(A2,I5,M3,E7,P12);(A2,I5,M3,E7,P13);(A2,I5,M3,E7,P14);(A2,I5,M3,E7,P15);(A2,I5,M3,E7,P16);(A2,I5,M3,E7,P17);(A2,I5,M3,E7,P18);(A2,I5,M3,E7,P19);(A2,I5,M3,E7,P20);(A2,I5,M3,E8,P1);(A2,I5,M3,E8,P2);(A2,I5,M3,E8,P3);(A2,I5,M3,E8,P4);(A2,I5,M3,E8,P5);(A2,I5,M3,E8,P6);(A2,I5,M3,E8,P7);(A2,I5,M3,E8,P8);(A2,I5,M3,E8,P9);(A2,I5,M3,E8,P10);(A2,I5,M3,E8,P11);(A2,I5,M3,E8,P12);(A2,I5,M3,E8,P13);(A2,I5,M3,E8,P14);(A2,I5,M3,E8,P15);(A2,I5,M3,E8,P16);(A2,I5,M3,E8,P17);(A2,I5,M3,E8,P18);(A2,I5,M3,E8,P19);(A2,I5,M3,E8,P20);(A2,I5,M3,E9,P1);(A2,I5,M3,E9,P2);(A2,I5,M3,E9,P3);(A2,I5,M3,E9,P4);(A2,I5,M3,E9,P5);(A2,I5,M3,E9,P6);(A2,I5,M3,E9,P7);(A2,I5,M3,E9,P8);(A2,I5,M3,E9,P9);(A2,I5,M3,E9,P10);(A2,I5,M3,E9,P11);(A2,I5,M3,E9,P12);(A2,I5,M3,E9,P13);(A2,I5,M3,E9,P14);(A2,I5,M3,E9,P15);(A2,I5,M3,E9,P16);(A2,I5,M3,E9,P17);(A2,I5,M3,E9,P18);(A2,I5,M3,E9,P19);(A2,I5,M3,E9,P20);(A2,I5,M4,E1,P1);(A2,I5,M4,E1,P2);(A2,I5,M4,E1,P3);(A2,I5,M4,E1,P4);(A2,I5,M4,E1,P5);(A2,I5,M4,E1,P6);(A2,I5,M4,E1,P7);(A2,I5,M4,E1,P8);(A2,I5,M4,E1,P9);(A2,I5,M4,E1,P10);(A2,I5,M4,E1,P11);(A2,I5,M4,E1,P12);(A2,I5,M4,E1,P13);(A2,I5,M4,E1,P14);(A2,I5,M4,E1,P15);(A2,I5,M4,E1,P16);(A2,I5,M4,E1,P17);(A2,I5,M4,E1,P18);(A2,I5,M4,E1,P19);(A2,I5,M4,E1,P20);(A2,I5,M4,E2,P1);(A2,I5,M4,E2,P2);(A2,I5,M4,E2,P3);(A2,I5,M4,E2,P4);(A2,I5,M4,E2,P5);(A2,I5,M4,E2,P6);(A2,I5,M4,E2,P7);(A2,I5,M4,E2,P8);(A2,I5,M4,E2,P9);(A2,I5,M4,E2,P10);(A2,I5,M4,E2,P11);(A2,I5,M4,E2,P12);(A2,I5,M4,E2,P13);(A2,I5,M4,E2,P14);(A2,I5,M4,E2,P15);(A2,I5,M4,E2,P16);(A2,I5,M4,E2,P17);(A2,I5,M4,E2,P18);(A2,I5,M4,E2,P19);(A2,I5,M4,E2,P20);(A2,I5,M4,E3,P1);(A2,I5,M4,E3,P2);(A2,I5,M4,E3,P3);(A2,I5,M4,E3,P4);(A2,I5,M4,E3,P5);(A2,I5,M4,E3,P6);(A2,I5,M4,E3,P7);(A2,I5,M4,E3,P8);(A2,I5,M4,E3,P9);(A2,I5,M4,E3,P10);(A2,I5,M4,E3,P11);(A2,I5,M4,E3,P12);(A2,I5,M4,E3,P13);(A2,I5,M4,E3,P14);(A2,I5,M4,E3,P15);(A2,I5,M4,E3,P16);(A2,I5,M4,E3,P17);(A2,I5,M4,E3,P18);(A2,I5,M4,E3,P19);(A2,I5,M4,E3,P20);(A2,I5,M4,E4,P1);(A2,I5,M4,E4,P2);(A2,I5,M4,E4,P3);(A2,I5,M4,E4,P4);(A2,I5,M4,E4,P5);(A2,I5,M4,E4,P6);(A2,I5,M4,E4,P7);(A2,I5,M4,E4,P8);(A2,I5,M4,E4,P9);(A2,I5,M4,E4,P10);(A2,I5,M4,E4,P11);(A2,I5,M4,E4,P12);(A2,I5,M4,E4,P13);(A2,I5,M4,E4,P14);(A2,I5,M4,E4,P15);(A2,I5,M4,E4,P16);(A2,I5,M4,E4,P17);(A2,I5,M4,E4,P18);(A2,I5,M4,E4,P19);(A2,I5,M4,E4,P20);(A2,I5,M4,E5,P1);(A2,I5,M4,E5,P2);(A2,I5,M4,E5,P3);(A2,I5,M4,E5,P4);(A2,I5,M4,E5,P5);(A2,I5,M4,E5,P6);(A2,I5,M4,E5,P7);(A2,I5,M4,E5,P8);(A2,I5,M4,E5,P9);(A2,I5,M4,E5,P10);(A2,I5,M4,E5,P11);(A2,I5,M4,E5,P12);(A2,I5,M4,E5,P13);(A2,I5,M4,E5,P14);(A2,I5,M4,E5,P15);(A2,I5,M4,E5,P16);(A2,I5,M4,E5,P17);(A2,I5,M4,E5,P18);(A2,I5,M4,E5,P19);(A2,I5,M4,E5,P20);(A2,I5,M4,E6,P1);(A2,I5,M4,E6,P2);(A2,I5,M4,E6,P3);(A2,I5,M4,E6,P4);(A2,I5,M4,E6,P5);(A2,I5,M4,E6,P6);(A2,I5,M4,E6,P7);(A2,I5,M4,E6,P8);(A2,I5,M4,E6,P9);(A2,I5,M4,E6,P10);(A2,I5,M4,E6,P11);(A2,I5,M4,E6,P12);(A2,I5,M4,E6,P13);(A2,I5,M4,E6,P14);(A2,I5,M4,E6,P15);(A2,I5,M4,E6,P16);(A2,I5,M4,E6,P17);(A2,I5,M4,E6,P18);(A2,I5,M4,E6,P19);(A2,I5,M4,E6,P20);(A2,I5,M4,E7,P1);(A2,I5,M4,E7,P2);(A2,I5,M4,E7,P3);(A2,I5,M4,E7,P4);(A2,I5,M4,E7,P5);(A2,I5,M4,E7,P6);(A2,I5,M4,E7,P7);(A2,I5,M4,E7,P8);(A2,I5,M4,E7,P9);(A2,I5,M4,E7,P10);(A2,I5,M4,E7,P11);(A2,I5,M4,E7,P12);(A2,I5,M4,E7,P13);(A2,I5,M4,E7,P14);(A2,I5,M4,E7,P15);(A2,I5,M4,E7,P16);(A2,I5,M4,E7,P17);(A2,I5,M4,E7,P18);(A2,I5,M4,E7,P19);(A2,I5,M4,E7,P20);(A2,I5,M4,E8,P1);(A2,I5,M4,E8,P2);(A2,I5,M4,E8,P3);(A2,I5,M4,E8,P4);(A2,I5,M4,E8,P5);(A2,I5,M4,E8,P6);(A2,I5,M4,E8,P7);(A2,I5,M4,E8,P8);(A2,I5,M4,E8,P9);(A2,I5,M4,E8,P10);(A2,I5,M4,E8,P11);(A2,I5,M4,E8,P12);(A2,I5,M4,E8,P13);(A2,I5,M4,E8,P14);(A2,I5,M4,E8,P15);(A2,I5,M4,E8,P16);(A2,I5,M4,E8,P17);(A2,I5,M4,E8,P18);(A2,I5,M4,E8,P19);(A2,I5,M4,E8,P20);(A2,I5,M4,E9,P1);(A2,I5,M4,E9,P2);(A2,I5,M4,E9,P3);(A2,I5,M4,E9,P4);(A2,I5,M4,E9,P5);(A2,I5,M4,E9,P6);(A2,I5,M4,E9,P7);(A2,I5,M4,E9,P8);(A2,I5,M4,E9,P9);(A2,I5,M4,E9,P10);(A2,I5,M4,E9,P11);(A2,I5,M4,E9,P12);(A2,I5,M4,E9,P13);(A2,I5,M4,E9,P14);(A2,I5,M4,E9,P15);(A2,I5,M4,E9,P16);(A2,I5,M4,E9,P17);(A2,I5,M4,E9,P18);(A2,I5,M4,E9,P19);(A2,I5,M4,E9,P20);(A2,I5,M5,E1,P1);(A2,I5,M5,E1,P2);(A2,I5,M5,E1,P3);(A2,I5,M5,E1,P4);(A2,I5,M5,E1,P5);(A2,I5,M5,E1,P6);(A2,I5,M5,E1,P7);(A2,I5,M5,E1,P8);(A2,I5,M5,E1,P9);(A2,I5,M5,E1,P10);(A2,I5,M5,E1,P11);(A2,I5,M5,E1,P12);(A2,I5,M5,E1,P13);(A2,I5,M5,E1,P14);(A2,I5,M5,E1,P15);(A2,I5,M5,E1,P16);(A2,I5,M5,E1,P17);(A2,I5,M5,E1,P18);(A2,I5,M5,E1,P19);(A2,I5,M5,E1,P20);(A2,I5,M5,E2,P1);(A2,I5,M5,E2,P2);(A2,I5,M5,E2,P3);(A2,I5,M5,E2,P4);(A2,I5,M5,E2,P5);(A2,I5,M5,E2,P6);(A2,I5,M5,E2,P7);(A2,I5,M5,E2,P8);(A2,I5,M5,E2,P9);(A2,I5,M5,E2,P10);(A2,I5,M5,E2,P11);(A2,I5,M5,E2,P12);(A2,I5,M5,E2,P13);(A2,I5,M5,E2,P14);(A2,I5,M5,E2,P15);(A2,I5,M5,E2,P16);(A2,I5,M5,E2,P17);(A2,I5,M5,E2,P18);(A2,I5,M5,E2,P19);(A2,I5,M5,E2,P20);(A2,I5,M5,E3,P1);(A2,I5,M5,E3,P2);(A2,I5,M5,E3,P3);(A2,I5,M5,E3,P4);(A2,I5,M5,E3,P5);(A2,I5,M5,E3,P6);(A2,I5,M5,E3,P7);(A2,I5,M5,E3,P8);(A2,I5,M5,E3,P9);(A2,I5,M5,E3,P10);(A2,I5,M5,E3,P11);(A2,I5,M5,E3,P12);(A2,I5,M5,E3,P13);(A2,I5,M5,E3,P14);(A2,I5,M5,E3,P15);(A2,I5,M5,E3,P16);(A2,I5,M5,E3,P17);(A2,I5,M5,E3,P18);(A2,I5,M5,E3,P19);(A2,I5,M5,E3,P20);(A2,I5,M5,E4,P1);(A2,I5,M5,E4,P2);(A2,I5,M5,E4,P3);(A2,I5,M5,E4,P4);(A2,I5,M5,E4,P5);(A2,I5,M5,E4,P6);(A2,I5,M5,E4,P7);(A2,I5,M5,E4,P8);(A2,I5,M5,E4,P9);(A2,I5,M5,E4,P10);(A2,I5,M5,E4,P11);(A2,I5,M5,E4,P12);(A2,I5,M5,E4,P13);(A2,I5,M5,E4,P14);(A2,I5,M5,E4,P15);(A2,I5,M5,E4,P16);(A2,I5,M5,E4,P17);(A2,I5,M5,E4,P18);(A2,I5,M5,E4,P19);(A2,I5,M5,E4,P20);(A2,I5,M5,E5,P1);(A2,I5,M5,E5,P2);(A2,I5,M5,E5,P3);(A2,I5,M5,E5,P4);(A2,I5,M5,E5,P5);(A2,I5,M5,E5,P6);(A2,I5,M5,E5,P7);(A2,I5,M5,E5,P8);(A2,I5,M5,E5,P9);(A2,I5,M5,E5,P10);(A2,I5,M5,E5,P11);(A2,I5,M5,E5,P12);(A2,I5,M5,E5,P13);(A2,I5,M5,E5,P14);(A2,I5,M5,E5,P15);(A2,I5,M5,E5,P16);(A2,I5,M5,E5,P17);(A2,I5,M5,E5,P18);(A2,I5,M5,E5,P19);(A2,I5,M5,E5,P20);(A2,I5,M5,E6,P1);(A2,I5,M5,E6,P2);(A2,I5,M5,E6,P3);(A2,I5,M5,E6,P4);(A2,I5,M5,E6,P5);(A2,I5,M5,E6,P6);(A2,I5,M5,E6,P7);(A2,I5,M5,E6,P8);(A2,I5,M5,E6,P9);(A2,I5,M5,E6,P10);(A2,I5,M5,E6,P11);(A2,I5,M5,E6,P12);(A2,I5,M5,E6,P13);(A2,I5,M5,E6,P14);(A2,I5,M5,E6,P15);(A2,I5,M5,E6,P16);(A2,I5,M5,E6,P17);(A2,I5,M5,E6,P18);(A2,I5,M5,E6,P19);(A2,I5,M5,E6,P20);(A2,I5,M5,E7,P1);(A2,I5,M5,E7,P2);(A2,I5,M5,E7,P3);(A2,I5,M5,E7,P4);(A2,I5,M5,E7,P5);(A2,I5,M5,E7,P6);(A2,I5,M5,E7,P7);(A2,I5,M5,E7,P8);(A2,I5,M5,E7,P9);(A2,I5,M5,E7,P10);(A2,I5,M5,E7,P11);(A2,I5,M5,E7,P12);(A2,I5,M5,E7,P13);(A2,I5,M5,E7,P14);(A2,I5,M5,E7,P15);(A2,I5,M5,E7,P16);(A2,I5,M5,E7,P17);(A2,I5,M5,E7,P18);(A2,I5,M5,E7,P19);(A2,I5,M5,E7,P20);(A2,I5,M5,E8,P1);(A2,I5,M5,E8,P2);(A2,I5,M5,E8,P3);(A2,I5,M5,E8,P4);(A2,I5,M5,E8,P5);(A2,I5,M5,E8,P6);(A2,I5,M5,E8,P7);(A2,I5,M5,E8,P8);(A2,I5,M5,E8,P9);(A2,I5,M5,E8,P10);(A2,I5,M5,E8,P11);(A2,I5,M5,E8,P12);(A2,I5,M5,E8,P13);(A2,I5,M5,E8,P14);(A2,I5,M5,E8,P15);(A2,I5,M5,E8,P16);(A2,I5,M5,E8,P17);(A2,I5,M5,E8,P18);(A2,I5,M5,E8,P19);(A2,I5,M5,E8,P20);(A2,I5,M5,E9,P1);(A2,I5,M5,E9,P2);(A2,I5,M5,E9,P3);(A2,I5,M5,E9,P4);(A2,I5,M5,E9,P5);(A2,I5,M5,E9,P6);(A2,I5,M5,E9,P7);(A2,I5,M5,E9,P8);(A2,I5,M5,E9,P9);(A2,I5,M5,E9,P10);(A2,I5,M5,E9,P11);(A2,I5,M5,E9,P12);(A2,I5,M5,E9,P13);(A2,I5,M5,E9,P14);(A2,I5,M5,E9,P15);(A2,I5,M5,E9,P16);(A2,I5,M5,E9,P17);(A2,I5,M5,E9,P18);(A2,I5,M5,E9,P19);(A2,I5,M5,E9,P20);(A2,I5,M6,E1,P1);(A2,I5,M6,E1,P2);(A2,I5,M6,E1,P3);(A2,I5,M6,E1,P4);(A2,I5,M6,E1,P5);(A2,I5,M6,E1,P6);(A2,I5,M6,E1,P7);(A2,I5,M6,E1,P8);(A2,I5,M6,E1,P9);(A2,I5,M6,E1,P10);(A2,I5,M6,E1,P11);(A2,I5,M6,E1,P12);(A2,I5,M6,E1,P13);(A2,I5,M6,E1,P14);(A2,I5,M6,E1,P15);(A2,I5,M6,E1,P16);(A2,I5,M6,E1,P17);(A2,I5,M6,E1,P18);(A2,I5,M6,E1,P19);(A2,I5,M6,E1,P20);(A2,I5,M6,E2,P1);(A2,I5,M6,E2,P2);(A2,I5,M6,E2,P3);(A2,I5,M6,E2,P4);(A2,I5,M6,E2,P5);(A2,I5,M6,E2,P6);(A2,I5,M6,E2,P7);(A2,I5,M6,E2,P8);(A2,I5,M6,E2,P9);(A2,I5,M6,E2,P10);(A2,I5,M6,E2,P11);(A2,I5,M6,E2,P12);(A2,I5,M6,E2,P13);(A2,I5,M6,E2,P14);(A2,I5,M6,E2,P15);(A2,I5,M6,E2,P16);(A2,I5,M6,E2,P17);(A2,I5,M6,E2,P18);(A2,I5,M6,E2,P19);(A2,I5,M6,E2,P20);(A2,I5,M6,E3,P1);(A2,I5,M6,E3,P2);(A2,I5,M6,E3,P3);(A2,I5,M6,E3,P4);(A2,I5,M6,E3,P5);(A2,I5,M6,E3,P6);(A2,I5,M6,E3,P7);(A2,I5,M6,E3,P8);(A2,I5,M6,E3,P9);(A2,I5,M6,E3,P10);(A2,I5,M6,E3,P11);(A2,I5,M6,E3,P12);(A2,I5,M6,E3,P13);(A2,I5,M6,E3,P14);(A2,I5,M6,E3,P15);(A2,I5,M6,E3,P16);(A2,I5,M6,E3,P17);(A2,I5,M6,E3,P18);(A2,I5,M6,E3,P19);(A2,I5,M6,E3,P20);(A2,I5,M6,E4,P1);(A2,I5,M6,E4,P2);(A2,I5,M6,E4,P3);(A2,I5,M6,E4,P4);(A2,I5,M6,E4,P5);(A2,I5,M6,E4,P6);(A2,I5,M6,E4,P7);(A2,I5,M6,E4,P8);(A2,I5,M6,E4,P9);(A2,I5,M6,E4,P10);(A2,I5,M6,E4,P11);(A2,I5,M6,E4,P12);(A2,I5,M6,E4,P13);(A2,I5,M6,E4,P14);(A2,I5,M6,E4,P15);(A2,I5,M6,E4,P16);(A2,I5,M6,E4,P17);(A2,I5,M6,E4,P18);(A2,I5,M6,E4,P19);(A2,I5,M6,E4,P20);(A2,I5,M6,E5,P1);(A2,I5,M6,E5,P2);(A2,I5,M6,E5,P3);(A2,I5,M6,E5,P4);(A2,I5,M6,E5,P5);(A2,I5,M6,E5,P6);(A2,I5,M6,E5,P7);(A2,I5,M6,E5,P8);(A2,I5,M6,E5,P9);(A2,I5,M6,E5,P10);(A2,I5,M6,E5,P11);(A2,I5,M6,E5,P12);(A2,I5,M6,E5,P13);(A2,I5,M6,E5,P14);(A2,I5,M6,E5,P15);(A2,I5,M6,E5,P16);(A2,I5,M6,E5,P17);(A2,I5,M6,E5,P18);(A2,I5,M6,E5,P19);(A2,I5,M6,E5,P20);(A2,I5,M6,E6,P1);(A2,I5,M6,E6,P2);(A2,I5,M6,E6,P3);(A2,I5,M6,E6,P4);(A2,I5,M6,E6,P5);(A2,I5,M6,E6,P6);(A2,I5,M6,E6,P7);(A2,I5,M6,E6,P8);(A2,I5,M6,E6,P9);(A2,I5,M6,E6,P10);(A2,I5,M6,E6,P11);(A2,I5,M6,E6,P12);(A2,I5,M6,E6,P13);(A2,I5,M6,E6,P14);(A2,I5,M6,E6,P15);(A2,I5,M6,E6,P16);(A2,I5,M6,E6,P17);(A2,I5,M6,E6,P18);(A2,I5,M6,E6,P19);(A2,I5,M6,E6,P20);(A2,I5,M6,E7,P1);(A2,I5,M6,E7,P2);(A2,I5,M6,E7,P3);(A2,I5,M6,E7,P4);(A2,I5,M6,E7,P5);(A2,I5,M6,E7,P6);(A2,I5,M6,E7,P7);(A2,I5,M6,E7,P8);(A2,I5,M6,E7,P9);(A2,I5,M6,E7,P10);(A2,I5,M6,E7,P11);(A2,I5,M6,E7,P12);(A2,I5,M6,E7,P13);(A2,I5,M6,E7,P14);(A2,I5,M6,E7,P15);(A2,I5,M6,E7,P16);(A2,I5,M6,E7,P17);(A2,I5,M6,E7,P18);(A2,I5,M6,E7,P19);(A2,I5,M6,E7,P20);(A2,I5,M6,E8,P1);(A2,I5,M6,E8,P2);(A2,I5,M6,E8,P3);(A2,I5,M6,E8,P4);(A2,I5,M6,E8,P5);(A2,I5,M6,E8,P6);(A2,I5,M6,E8,P7);(A2,I5,M6,E8,P8);(A2,I5,M6,E8,P9);(A2,I5,M6,E8,P10);(A2,I5,M6,E8,P11);(A2,I5,M6,E8,P12);(A2,I5,M6,E8,P13);(A2,I5,M6,E8,P14);(A2,I5,M6,E8,P15);(A2,I5,M6,E8,P16);(A2,I5,M6,E8,P17);(A2,I5,M6,E8,P18);(A2,I5,M6,E8,P19);(A2,I5,M6,E8,P20);(A2,I5,M6,E9,P1);(A2,I5,M6,E9,P2);(A2,I5,M6,E9,P3);(A2,I5,M6,E9,P4);(A2,I5,M6,E9,P5);(A2,I5,M6,E9,P6);(A2,I5,M6,E9,P7);(A2,I5,M6,E9,P8);(A2,I5,M6,E9,P9);(A2,I5,M6,E9,P10);(A2,I5,M6,E9,P11);(A2,I5,M6,E9,P12);(A2,I5,M6,E9,P13);(A2,I5,M6,E9,P14);(A2,I5,M6,E9,P15);(A2,I5,M6,E9,P16);(A2,I5,M6,E9,P17);(A2,I5,M6,E9,P18);(A2,I5,M6,E9,P19);(A2,I5,M6,E9,P20);(A2,I6,M1,E1,P1);(A2,I6,M1,E1,P2);(A2,I6,M1,E1,P3);(A2,I6,M1,E1,P4);(A2,I6,M1,E1,P5);(A2,I6,M1,E1,P6);(A2,I6,M1,E1,P7);(A2,I6,M1,E1,P8);(A2,I6,M1,E1,P9);(A2,I6,M1,E1,P10);(A2,I6,M1,E1,P11);(A2,I6,M1,E1,P12);(A2,I6,M1,E1,P13);(A2,I6,M1,E1,P14);(A2,I6,M1,E1,P15);(A2,I6,M1,E1,P16);(A2,I6,M1,E1,P17);(A2,I6,M1,E1,P18);(A2,I6,M1,E1,P19);(A2,I6,M1,E1,P20);(A2,I6,M1,E2,P1);(A2,I6,M1,E2,P2);(A2,I6,M1,E2,P3);(A2,I6,M1,E2,P4);(A2,I6,M1,E2,P5);(A2,I6,M1,E2,P6);(A2,I6,M1,E2,P7);(A2,I6,M1,E2,P8);(A2,I6,M1,E2,P9);(A2,I6,M1,E2,P10);(A2,I6,M1,E2,P11);(A2,I6,M1,E2,P12);(A2,I6,M1,E2,P13);(A2,I6,M1,E2,P14);(A2,I6,M1,E2,P15);(A2,I6,M1,E2,P16);(A2,I6,M1,E2,P17);(A2,I6,M1,E2,P18);(A2,I6,M1,E2,P19);(A2,I6,M1,E2,P20);(A2,I6,M1,E3,P1);(A2,I6,M1,E3,P2);(A2,I6,M1,E3,P3);(A2,I6,M1,E3,P4);(A2,I6,M1,E3,P5);(A2,I6,M1,E3,P6);(A2,I6,M1,E3,P7);(A2,I6,M1,E3,P8);(A2,I6,M1,E3,P9);(A2,I6,M1,E3,P10);(A2,I6,M1,E3,P11);(A2,I6,M1,E3,P12);(A2,I6,M1,E3,P13);(A2,I6,M1,E3,P14);(A2,I6,M1,E3,P15);(A2,I6,M1,E3,P16);(A2,I6,M1,E3,P17);(A2,I6,M1,E3,P18);(A2,I6,M1,E3,P19);(A2,I6,M1,E3,P20);(A2,I6,M1,E4,P1);(A2,I6,M1,E4,P2);(A2,I6,M1,E4,P3);(A2,I6,M1,E4,P4);(A2,I6,M1,E4,P5);(A2,I6,M1,E4,P6);(A2,I6,M1,E4,P7);(A2,I6,M1,E4,P8);(A2,I6,M1,E4,P9);(A2,I6,M1,E4,P10);(A2,I6,M1,E4,P11);(A2,I6,M1,E4,P12);(A2,I6,M1,E4,P13);(A2,I6,M1,E4,P14);(A2,I6,M1,E4,P15);(A2,I6,M1,E4,P16);(A2,I6,M1,E4,P17);(A2,I6,M1,E4,P18);(A2,I6,M1,E4,P19);(A2,I6,M1,E4,P20);(A2,I6,M1,E5,P1);(A2,I6,M1,E5,P2);(A2,I6,M1,E5,P3);(A2,I6,M1,E5,P4);(A2,I6,M1,E5,P5);(A2,I6,M1,E5,P6);(A2,I6,M1,E5,P7);(A2,I6,M1,E5,P8);(A2,I6,M1,E5,P9);(A2,I6,M1,E5,P10);(A2,I6,M1,E5,P11);(A2,I6,M1,E5,P12);(A2,I6,M1,E5,P13);(A2,I6,M1,E5,P14);(A2,I6,M1,E5,P15);(A2,I6,M1,E5,P16);(A2,I6,M1,E5,P17);(A2,I6,M1,E5,P18);(A2,I6,M1,E5,P19);(A2,I6,M1,E5,P20);(A2,I6,M1,E6,P1);(A2,I6,M1,E6,P2);(A2,I6,M1,E6,P3);(A2,I6,M1,E6,P4);(A2,I6,M1,E6,P5);(A2,I6,M1,E6,P6);(A2,I6,M1,E6,P7);(A2,I6,M1,E6,P8);(A2,I6,M1,E6,P9);(A2,I6,M1,E6,P10);(A2,I6,M1,E6,P11);(A2,I6,M1,E6,P12);(A2,I6,M1,E6,P13);(A2,I6,M1,E6,P14);(A2,I6,M1,E6,P15);(A2,I6,M1,E6,P16);(A2,I6,M1,E6,P17);(A2,I6,M1,E6,P18);(A2,I6,M1,E6,P19);(A2,I6,M1,E6,P20);(A2,I6,M1,E7,P1);(A2,I6,M1,E7,P2);(A2,I6,M1,E7,P3);(A2,I6,M1,E7,P4);(A2,I6,M1,E7,P5);(A2,I6,M1,E7,P6);(A2,I6,M1,E7,P7);(A2,I6,M1,E7,P8);(A2,I6,M1,E7,P9);(A2,I6,M1,E7,P10);(A2,I6,M1,E7,P11);(A2,I6,M1,E7,P12);(A2,I6,M1,E7,P13);(A2,I6,M1,E7,P14);(A2,I6,M1,E7,P15);(A2,I6,M1,E7,P16);(A2,I6,M1,E7,P17);(A2,I6,M1,E7,P18);(A2,I6,M1,E7,P19);(A2,I6,M1,E7,P20);(A2,I6,M1,E8,P1);(A2,I6,M1,E8,P2);(A2,I6,M1,E8,P3);(A2,I6,M1,E8,P4);(A2,I6,M1,E8,P5);(A2,I6,M1,E8,P6);(A2,I6,M1,E8,P7);(A2,I6,M1,E8,P8);(A2,I6,M1,E8,P9);(A2,I6,M1,E8,P10);(A2,I6,M1,E8,P11);(A2,I6,M1,E8,P12);(A2,I6,M1,E8,P13);(A2,I6,M1,E8,P14);(A2,I6,M1,E8,P15);(A2,I6,M1,E8,P16);(A2,I6,M1,E8,P17);(A2,I6,M1,E8,P18);(A2,I6,M1,E8,P19);(A2,I6,M1,E8,P20);(A2,I6,M1,E9,P1);(A2,I6,M1,E9,P2);(A2,I6,M1,E9,P3);(A2,I6,M1,E9,P4);(A2,I6,M1,E9,P5);(A2,I6,M1,E9,P6);(A2,I6,M1,E9,P7);(A2,I6,M1,E9,P8);(A2,I6,M1,E9,P9);(A2,I6,M1,E9,P10);(A2,I6,M1,E9,P11);(A2,I6,M1,E9,P12);(A2,I6,M1,E9,P13);(A2,I6,M1,E9,P14);(A2,I6,M1,E9,P15);(A2,I6,M1,E9,P16);(A2,I6,M1,E9,P17);(A2,I6,M1,E9,P18);(A2,I6,M1,E9,P19);(A2,I6,M1,E9,P20);(A2,I6,M2,E1,P1);(A2,I6,M2,E1,P2);(A2,I6,M2,E1,P3);(A2,I6,M2,E1,P4);(A2,I6,M2,E1,P5);(A2,I6,M2,E1,P6);(A2,I6,M2,E1,P7);(A2,I6,M2,E1,P8);(A2,I6,M2,E1,P9);(A2,I6,M2,E1,P10);(A2,I6,M2,E1,P11);(A2,I6,M2,E1,P12);(A2,I6,M2,E1,P13);(A2,I6,M2,E1,P14);(A2,I6,M2,E1,P15);(A2,I6,M2,E1,P16);(A2,I6,M2,E1,P17);(A2,I6,M2,E1,P18);(A2,I6,M2,E1,P19);(A2,I6,M2,E1,P20);(A2,I6,M2,E2,P1);(A2,I6,M2,E2,P2);(A2,I6,M2,E2,P3);(A2,I6,M2,E2,P4);(A2,I6,M2,E2,P5);(A2,I6,M2,E2,P6);(A2,I6,M2,E2,P7);(A2,I6,M2,E2,P8);(A2,I6,M2,E2,P9);(A2,I6,M2,E2,P10);(A2,I6,M2,E2,P11);(A2,I6,M2,E2,P12);(A2,I6,M2,E2,P13);(A2,I6,M2,E2,P14);(A2,I6,M2,E2,P15);(A2,I6,M2,E2,P16);(A2,I6,M2,E2,P17);(A2,I6,M2,E2,P18);(A2,I6,M2,E2,P19);(A2,I6,M2,E2,P20);(A2,I6,M2,E3,P1);(A2,I6,M2,E3,P2);(A2,I6,M2,E3,P3);(A2,I6,M2,E3,P4);(A2,I6,M2,E3,P5);(A2,I6,M2,E3,P6);(A2,I6,M2,E3,P7);(A2,I6,M2,E3,P8);(A2,I6,M2,E3,P9);(A2,I6,M2,E3,P10);(A2,I6,M2,E3,P11);(A2,I6,M2,E3,P12);(A2,I6,M2,E3,P13);(A2,I6,M2,E3,P14);(A2,I6,M2,E3,P15);(A2,I6,M2,E3,P16);(A2,I6,M2,E3,P17);(A2,I6,M2,E3,P18);(A2,I6,M2,E3,P19);(A2,I6,M2,E3,P20);(A2,I6,M2,E4,P1);(A2,I6,M2,E4,P2);(A2,I6,M2,E4,P3);(A2,I6,M2,E4,P4);(A2,I6,M2,E4,P5);(A2,I6,M2,E4,P6);(A2,I6,M2,E4,P7);(A2,I6,M2,E4,P8);(A2,I6,M2,E4,P9);(A2,I6,M2,E4,P10);(A2,I6,M2,E4,P11);(A2,I6,M2,E4,P12);(A2,I6,M2,E4,P13);(A2,I6,M2,E4,P14);(A2,I6,M2,E4,P15);(A2,I6,M2,E4,P16);(A2,I6,M2,E4,P17);(A2,I6,M2,E4,P18);(A2,I6,M2,E4,P19);(A2,I6,M2,E4,P20);(A2,I6,M2,E5,P1);(A2,I6,M2,E5,P2);(A2,I6,M2,E5,P3);(A2,I6,M2,E5,P4);(A2,I6,M2,E5,P5);(A2,I6,M2,E5,P6);(A2,I6,M2,E5,P7);(A2,I6,M2,E5,P8);(A2,I6,M2,E5,P9);(A2,I6,M2,E5,P10);(A2,I6,M2,E5,P11);(A2,I6,M2,E5,P12);(A2,I6,M2,E5,P13);(A2,I6,M2,E5,P14);(A2,I6,M2,E5,P15);(A2,I6,M2,E5,P16);(A2,I6,M2,E5,P17);(A2,I6,M2,E5,P18);(A2,I6,M2,E5,P19);(A2,I6,M2,E5,P20);(A2,I6,M2,E6,P1);(A2,I6,M2,E6,P2);(A2,I6,M2,E6,P3);(A2,I6,M2,E6,P4);(A2,I6,M2,E6,P5);(A2,I6,M2,E6,P6);(A2,I6,M2,E6,P7);(A2,I6,M2,E6,P8);(A2,I6,M2,E6,P9);(A2,I6,M2,E6,P10);(A2,I6,M2,E6,P11);(A2,I6,M2,E6,P12);(A2,I6,M2,E6,P13);(A2,I6,M2,E6,P14);(A2,I6,M2,E6,P15);(A2,I6,M2,E6,P16);(A2,I6,M2,E6,P17);(A2,I6,M2,E6,P18);(A2,I6,M2,E6,P19);(A2,I6,M2,E6,P20);(A2,I6,M2,E7,P1);(A2,I6,M2,E7,P2);(A2,I6,M2,E7,P3);(A2,I6,M2,E7,P4);(A2,I6,M2,E7,P5);(A2,I6,M2,E7,P6);(A2,I6,M2,E7,P7);(A2,I6,M2,E7,P8);(A2,I6,M2,E7,P9);(A2,I6,M2,E7,P10);(A2,I6,M2,E7,P11);(A2,I6,M2,E7,P12);(A2,I6,M2,E7,P13);(A2,I6,M2,E7,P14);(A2,I6,M2,E7,P15);(A2,I6,M2,E7,P16);(A2,I6,M2,E7,P17);(A2,I6,M2,E7,P18);(A2,I6,M2,E7,P19);(A2,I6,M2,E7,P20);(A2,I6,M2,E8,P1);(A2,I6,M2,E8,P2);(A2,I6,M2,E8,P3);(A2,I6,M2,E8,P4);(A2,I6,M2,E8,P5);(A2,I6,M2,E8,P6);(A2,I6,M2,E8,P7);(A2,I6,M2,E8,P8);(A2,I6,M2,E8,P9);(A2,I6,M2,E8,P10);(A2,I6,M2,E8,P11);(A2,I6,M2,E8,P12);(A2,I6,M2,E8,P13);(A2,I6,M2,E8,P14);(A2,I6,M2,E8,P15);(A2,I6,M2,E8,P16);(A2,I6,M2,E8,P17);(A2,I6,M2,E8,P18);(A2,I6,M2,E8,P19);(A2,I6,M2,E8,P20);(A2,I6,M2,E9,P1);(A2,I6,M2,E9,P2);(A2,I6,M2,E9,P3);(A2,I6,M2,E9,P4);(A2,I6,M2,E9,P5);(A2,I6,M2,E9,P6);(A2,I6,M2,E9,P7);(A2,I6,M2,E9,P8);(A2,I6,M2,E9,P9);(A2,I6,M2,E9,P10);(A2,I6,M2,E9,P11);(A2,I6,M2,E9,P12);(A2,I6,M2,E9,P13);(A2,I6,M2,E9,P14);(A2,I6,M2,E9,P15);(A2,I6,M2,E9,P16);(A2,I6,M2,E9,P17);(A2,I6,M2,E9,P18);(A2,I6,M2,E9,P19);(A2,I6,M2,E9,P20);(A2,I6,M3,E1,P1);(A2,I6,M3,E1,P2);(A2,I6,M3,E1,P3);(A2,I6,M3,E1,P4);(A2,I6,M3,E1,P5);(A2,I6,M3,E1,P6);(A2,I6,M3,E1,P7);(A2,I6,M3,E1,P8);(A2,I6,M3,E1,P9);(A2,I6,M3,E1,P10);(A2,I6,M3,E1,P11);(A2,I6,M3,E1,P12);(A2,I6,M3,E1,P13);(A2,I6,M3,E1,P14);(A2,I6,M3,E1,P15);(A2,I6,M3,E1,P16);(A2,I6,M3,E1,P17);(A2,I6,M3,E1,P18);(A2,I6,M3,E1,P19);(A2,I6,M3,E1,P20);(A2,I6,M3,E2,P1);(A2,I6,M3,E2,P2);(A2,I6,M3,E2,P3);(A2,I6,M3,E2,P4);(A2,I6,M3,E2,P5);(A2,I6,M3,E2,P6);(A2,I6,M3,E2,P7);(A2,I6,M3,E2,P8);(A2,I6,M3,E2,P9);(A2,I6,M3,E2,P10);(A2,I6,M3,E2,P11);(A2,I6,M3,E2,P12);(A2,I6,M3,E2,P13);(A2,I6,M3,E2,P14);(A2,I6,M3,E2,P15);(A2,I6,M3,E2,P16);(A2,I6,M3,E2,P17);(A2,I6,M3,E2,P18);(A2,I6,M3,E2,P19);(A2,I6,M3,E2,P20);(A2,I6,M3,E3,P1);(A2,I6,M3,E3,P2);(A2,I6,M3,E3,P3);(A2,I6,M3,E3,P4);(A2,I6,M3,E3,P5);(A2,I6,M3,E3,P6);(A2,I6,M3,E3,P7);(A2,I6,M3,E3,P8);(A2,I6,M3,E3,P9);(A2,I6,M3,E3,P10);(A2,I6,M3,E3,P11);(A2,I6,M3,E3,P12);(A2,I6,M3,E3,P13);(A2,I6,M3,E3,P14);(A2,I6,M3,E3,P15);(A2,I6,M3,E3,P16);(A2,I6,M3,E3,P17);(A2,I6,M3,E3,P18);(A2,I6,M3,E3,P19);(A2,I6,M3,E3,P20);(A2,I6,M3,E4,P1);(A2,I6,M3,E4,P2);(A2,I6,M3,E4,P3);(A2,I6,M3,E4,P4);(A2,I6,M3,E4,P5);(A2,I6,M3,E4,P6);(A2,I6,M3,E4,P7);(A2,I6,M3,E4,P8);(A2,I6,M3,E4,P9);(A2,I6,M3,E4,P10);(A2,I6,M3,E4,P11);(A2,I6,M3,E4,P12);(A2,I6,M3,E4,P13);(A2,I6,M3,E4,P14);(A2,I6,M3,E4,P15);(A2,I6,M3,E4,P16);(A2,I6,M3,E4,P17);(A2,I6,M3,E4,P18);(A2,I6,M3,E4,P19);(A2,I6,M3,E4,P20);(A2,I6,M3,E5,P1);(A2,I6,M3,E5,P2);(A2,I6,M3,E5,P3);(A2,I6,M3,E5,P4);(A2,I6,M3,E5,P5);(A2,I6,M3,E5,P6);(A2,I6,M3,E5,P7);(A2,I6,M3,E5,P8);(A2,I6,M3,E5,P9);(A2,I6,M3,E5,P10);(A2,I6,M3,E5,P11);(A2,I6,M3,E5,P12);(A2,I6,M3,E5,P13);(A2,I6,M3,E5,P14);(A2,I6,M3,E5,P15);(A2,I6,M3,E5,P16);(A2,I6,M3,E5,P17);(A2,I6,M3,E5,P18);(A2,I6,M3,E5,P19);(A2,I6,M3,E5,P20);(A2,I6,M3,E6,P1);(A2,I6,M3,E6,P2);(A2,I6,M3,E6,P3);(A2,I6,M3,E6,P4);(A2,I6,M3,E6,P5);(A2,I6,M3,E6,P6);(A2,I6,M3,E6,P7);(A2,I6,M3,E6,P8);(A2,I6,M3,E6,P9);(A2,I6,M3,E6,P10);(A2,I6,M3,E6,P11);(A2,I6,M3,E6,P12);(A2,I6,M3,E6,P13);(A2,I6,M3,E6,P14);(A2,I6,M3,E6,P15);(A2,I6,M3,E6,P16);(A2,I6,M3,E6,P17);(A2,I6,M3,E6,P18);(A2,I6,M3,E6,P19);(A2,I6,M3,E6,P20);(A2,I6,M3,E7,P1);(A2,I6,M3,E7,P2);(A2,I6,M3,E7,P3);(A2,I6,M3,E7,P4);(A2,I6,M3,E7,P5);(A2,I6,M3,E7,P6);(A2,I6,M3,E7,P7);(A2,I6,M3,E7,P8);(A2,I6,M3,E7,P9);(A2,I6,M3,E7,P10);(A2,I6,M3,E7,P11);(A2,I6,M3,E7,P12);(A2,I6,M3,E7,P13);(A2,I6,M3,E7,P14);(A2,I6,M3,E7,P15);(A2,I6,M3,E7,P16);(A2,I6,M3,E7,P17);(A2,I6,M3,E7,P18);(A2,I6,M3,E7,P19);(A2,I6,M3,E7,P20);(A2,I6,M3,E8,P1);(A2,I6,M3,E8,P2);(A2,I6,M3,E8,P3);(A2,I6,M3,E8,P4);(A2,I6,M3,E8,P5);(A2,I6,M3,E8,P6);(A2,I6,M3,E8,P7);(A2,I6,M3,E8,P8);(A2,I6,M3,E8,P9);(A2,I6,M3,E8,P10);(A2,I6,M3,E8,P11);(A2,I6,M3,E8,P12);(A2,I6,M3,E8,P13);(A2,I6,M3,E8,P14);(A2,I6,M3,E8,P15);(A2,I6,M3,E8,P16);(A2,I6,M3,E8,P17);(A2,I6,M3,E8,P18);(A2,I6,M3,E8,P19);(A2,I6,M3,E8,P20);(A2,I6,M3,E9,P1);(A2,I6,M3,E9,P2);(A2,I6,M3,E9,P3);(A2,I6,M3,E9,P4);(A2,I6,M3,E9,P5);(A2,I6,M3,E9,P6);(A2,I6,M3,E9,P7);(A2,I6,M3,E9,P8);(A2,I6,M3,E9,P9);(A2,I6,M3,E9,P10);(A2,I6,M3,E9,P11);(A2,I6,M3,E9,P12);(A2,I6,M3,E9,P13);(A2,I6,M3,E9,P14);(A2,I6,M3,E9,P15);(A2,I6,M3,E9,P16);(A2,I6,M3,E9,P17);(A2,I6,M3,E9,P18);(A2,I6,M3,E9,P19);(A2,I6,M3,E9,P20);(A2,I6,M4,E1,P1);(A2,I6,M4,E1,P2);(A2,I6,M4,E1,P3);(A2,I6,M4,E1,P4);(A2,I6,M4,E1,P5);(A2,I6,M4,E1,P6);(A2,I6,M4,E1,P7);(A2,I6,M4,E1,P8);(A2,I6,M4,E1,P9);(A2,I6,M4,E1,P10);(A2,I6,M4,E1,P11);(A2,I6,M4,E1,P12);(A2,I6,M4,E1,P13);(A2,I6,M4,E1,P14);(A2,I6,M4,E1,P15);(A2,I6,M4,E1,P16);(A2,I6,M4,E1,P17);(A2,I6,M4,E1,P18);(A2,I6,M4,E1,P19);(A2,I6,M4,E1,P20);(A2,I6,M4,E2,P1);(A2,I6,M4,E2,P2);(A2,I6,M4,E2,P3);(A2,I6,M4,E2,P4);(A2,I6,M4,E2,P5);(A2,I6,M4,E2,P6);(A2,I6,M4,E2,P7);(A2,I6,M4,E2,P8);(A2,I6,M4,E2,P9);(A2,I6,M4,E2,P10);(A2,I6,M4,E2,P11);(A2,I6,M4,E2,P12);(A2,I6,M4,E2,P13);(A2,I6,M4,E2,P14);(A2,I6,M4,E2,P15);(A2,I6,M4,E2,P16);(A2,I6,M4,E2,P17);(A2,I6,M4,E2,P18);(A2,I6,M4,E2,P19);(A2,I6,M4,E2,P20);(A2,I6,M4,E3,P1);(A2,I6,M4,E3,P2);(A2,I6,M4,E3,P3);(A2,I6,M4,E3,P4);(A2,I6,M4,E3,P5);(A2,I6,M4,E3,P6);(A2,I6,M4,E3,P7);(A2,I6,M4,E3,P8);(A2,I6,M4,E3,P9);(A2,I6,M4,E3,P10);(A2,I6,M4,E3,P11);(A2,I6,M4,E3,P12);(A2,I6,M4,E3,P13);(A2,I6,M4,E3,P14);(A2,I6,M4,E3,P15);(A2,I6,M4,E3,P16);(A2,I6,M4,E3,P17);(A2,I6,M4,E3,P18);(A2,I6,M4,E3,P19);(A2,I6,M4,E3,P20);(A2,I6,M4,E4,P1);(A2,I6,M4,E4,P2);(A2,I6,M4,E4,P3);(A2,I6,M4,E4,P4);(A2,I6,M4,E4,P5);(A2,I6,M4,E4,P6);(A2,I6,M4,E4,P7);(A2,I6,M4,E4,P8);(A2,I6,M4,E4,P9);(A2,I6,M4,E4,P10);(A2,I6,M4,E4,P11);(A2,I6,M4,E4,P12);(A2,I6,M4,E4,P13);(A2,I6,M4,E4,P14);(A2,I6,M4,E4,P15);(A2,I6,M4,E4,P16);(A2,I6,M4,E4,P17);(A2,I6,M4,E4,P18);(A2,I6,M4,E4,P19);(A2,I6,M4,E4,P20);(A2,I6,M4,E5,P1);(A2,I6,M4,E5,P2);(A2,I6,M4,E5,P3);(A2,I6,M4,E5,P4);(A2,I6,M4,E5,P5);(A2,I6,M4,E5,P6);(A2,I6,M4,E5,P7);(A2,I6,M4,E5,P8);(A2,I6,M4,E5,P9);(A2,I6,M4,E5,P10);(A2,I6,M4,E5,P11);(A2,I6,M4,E5,P12);(A2,I6,M4,E5,P13);(A2,I6,M4,E5,P14);(A2,I6,M4,E5,P15);(A2,I6,M4,E5,P16);(A2,I6,M4,E5,P17);(A2,I6,M4,E5,P18);(A2,I6,M4,E5,P19);(A2,I6,M4,E5,P20);(A2,I6,M4,E6,P1);(A2,I6,M4,E6,P2);(A2,I6,M4,E6,P3);(A2,I6,M4,E6,P4);(A2,I6,M4,E6,P5);(A2,I6,M4,E6,P6);(A2,I6,M4,E6,P7);(A2,I6,M4,E6,P8);(A2,I6,M4,E6,P9);(A2,I6,M4,E6,P10);(A2,I6,M4,E6,P11);(A2,I6,M4,E6,P12);(A2,I6,M4,E6,P13);(A2,I6,M4,E6,P14);(A2,I6,M4,E6,P15);(A2,I6,M4,E6,P16);(A2,I6,M4,E6,P17);(A2,I6,M4,E6,P18);(A2,I6,M4,E6,P19);(A2,I6,M4,E6,P20);(A2,I6,M4,E7,P1);(A2,I6,M4,E7,P2);(A2,I6,M4,E7,P3);(A2,I6,M4,E7,P4);(A2,I6,M4,E7,P5);(A2,I6,M4,E7,P6);(A2,I6,M4,E7,P7);(A2,I6,M4,E7,P8);(A2,I6,M4,E7,P9);(A2,I6,M4,E7,P10);(A2,I6,M4,E7,P11);(A2,I6,M4,E7,P12);(A2,I6,M4,E7,P13);(A2,I6,M4,E7,P14);(A2,I6,M4,E7,P15);(A2,I6,M4,E7,P16);(A2,I6,M4,E7,P17);(A2,I6,M4,E7,P18);(A2,I6,M4,E7,P19);(A2,I6,M4,E7,P20);(A2,I6,M4,E8,P1);(A2,I6,M4,E8,P2);(A2,I6,M4,E8,P3);(A2,I6,M4,E8,P4);(A2,I6,M4,E8,P5);(A2,I6,M4,E8,P6);(A2,I6,M4,E8,P7);(A2,I6,M4,E8,P8);(A2,I6,M4,E8,P9);(A2,I6,M4,E8,P10);(A2,I6,M4,E8,P11);(A2,I6,M4,E8,P12);(A2,I6,M4,E8,P13);(A2,I6,M4,E8,P14);(A2,I6,M4,E8,P15);(A2,I6,M4,E8,P16);(A2,I6,M4,E8,P17);(A2,I6,M4,E8,P18);(A2,I6,M4,E8,P19);(A2,I6,M4,E8,P20);(A2,I6,M4,E9,P1);(A2,I6,M4,E9,P2);(A2,I6,M4,E9,P3);(A2,I6,M4,E9,P4);(A2,I6,M4,E9,P5);(A2,I6,M4,E9,P6);(A2,I6,M4,E9,P7);(A2,I6,M4,E9,P8);(A2,I6,M4,E9,P9);(A2,I6,M4,E9,P10);(A2,I6,M4,E9,P11);(A2,I6,M4,E9,P12);(A2,I6,M4,E9,P13);(A2,I6,M4,E9,P14);(A2,I6,M4,E9,P15);(A2,I6,M4,E9,P16);(A2,I6,M4,E9,P17);(A2,I6,M4,E9,P18);(A2,I6,M4,E9,P19);(A2,I6,M4,E9,P20);(A2,I6,M5,E1,P1);(A2,I6,M5,E1,P2);(A2,I6,M5,E1,P3);(A2,I6,M5,E1,P4);(A2,I6,M5,E1,P5);(A2,I6,M5,E1,P6);(A2,I6,M5,E1,P7);(A2,I6,M5,E1,P8);(A2,I6,M5,E1,P9);(A2,I6,M5,E1,P10);(A2,I6,M5,E1,P11);(A2,I6,M5,E1,P12);(A2,I6,M5,E1,P13);(A2,I6,M5,E1,P14);(A2,I6,M5,E1,P15);(A2,I6,M5,E1,P16);(A2,I6,M5,E1,P17);(A2,I6,M5,E1,P18);(A2,I6,M5,E1,P19);(A2,I6,M5,E1,P20);(A2,I6,M5,E2,P1);(A2,I6,M5,E2,P2);(A2,I6,M5,E2,P3);(A2,I6,M5,E2,P4);(A2,I6,M5,E2,P5);(A2,I6,M5,E2,P6);(A2,I6,M5,E2,P7);(A2,I6,M5,E2,P8);(A2,I6,M5,E2,P9);(A2,I6,M5,E2,P10);(A2,I6,M5,E2,P11);(A2,I6,M5,E2,P12);(A2,I6,M5,E2,P13);(A2,I6,M5,E2,P14);(A2,I6,M5,E2,P15);(A2,I6,M5,E2,P16);(A2,I6,M5,E2,P17);(A2,I6,M5,E2,P18);(A2,I6,M5,E2,P19);(A2,I6,M5,E2,P20);(A2,I6,M5,E3,P1);(A2,I6,M5,E3,P2);(A2,I6,M5,E3,P3);(A2,I6,M5,E3,P4);(A2,I6,M5,E3,P5);(A2,I6,M5,E3,P6);(A2,I6,M5,E3,P7);(A2,I6,M5,E3,P8);(A2,I6,M5,E3,P9);(A2,I6,M5,E3,P10);(A2,I6,M5,E3,P11);(A2,I6,M5,E3,P12);(A2,I6,M5,E3,P13);(A2,I6,M5,E3,P14);(A2,I6,M5,E3,P15);(A2,I6,M5,E3,P16);(A2,I6,M5,E3,P17);(A2,I6,M5,E3,P18);(A2,I6,M5,E3,P19);(A2,I6,M5,E3,P20);(A2,I6,M5,E4,P1);(A2,I6,M5,E4,P2);(A2,I6,M5,E4,P3);(A2,I6,M5,E4,P4);(A2,I6,M5,E4,P5);(A2,I6,M5,E4,P6);(A2,I6,M5,E4,P7);(A2,I6,M5,E4,P8);(A2,I6,M5,E4,P9);(A2,I6,M5,E4,P10);(A2,I6,M5,E4,P11);(A2,I6,M5,E4,P12);(A2,I6,M5,E4,P13);(A2,I6,M5,E4,P14);(A2,I6,M5,E4,P15);(A2,I6,M5,E4,P16);(A2,I6,M5,E4,P17);(A2,I6,M5,E4,P18);(A2,I6,M5,E4,P19);(A2,I6,M5,E4,P20);(A2,I6,M5,E5,P1);(A2,I6,M5,E5,P2);(A2,I6,M5,E5,P3);(A2,I6,M5,E5,P4);(A2,I6,M5,E5,P5);(A2,I6,M5,E5,P6);(A2,I6,M5,E5,P7);(A2,I6,M5,E5,P8);(A2,I6,M5,E5,P9);(A2,I6,M5,E5,P10);(A2,I6,M5,E5,P11);(A2,I6,M5,E5,P12);(A2,I6,M5,E5,P13);(A2,I6,M5,E5,P14);(A2,I6,M5,E5,P15);(A2,I6,M5,E5,P16);(A2,I6,M5,E5,P17);(A2,I6,M5,E5,P18);(A2,I6,M5,E5,P19);(A2,I6,M5,E5,P20);(A2,I6,M5,E6,P1);(A2,I6,M5,E6,P2);(A2,I6,M5,E6,P3);(A2,I6,M5,E6,P4);(A2,I6,M5,E6,P5);(A2,I6,M5,E6,P6);(A2,I6,M5,E6,P7);(A2,I6,M5,E6,P8);(A2,I6,M5,E6,P9);(A2,I6,M5,E6,P10);(A2,I6,M5,E6,P11);(A2,I6,M5,E6,P12);(A2,I6,M5,E6,P13);(A2,I6,M5,E6,P14);(A2,I6,M5,E6,P15);(A2,I6,M5,E6,P16);(A2,I6,M5,E6,P17);(A2,I6,M5,E6,P18);(A2,I6,M5,E6,P19);(A2,I6,M5,E6,P20);(A2,I6,M5,E7,P1);(A2,I6,M5,E7,P2);(A2,I6,M5,E7,P3);(A2,I6,M5,E7,P4);(A2,I6,M5,E7,P5);(A2,I6,M5,E7,P6);(A2,I6,M5,E7,P7);(A2,I6,M5,E7,P8);(A2,I6,M5,E7,P9);(A2,I6,M5,E7,P10);(A2,I6,M5,E7,P11);(A2,I6,M5,E7,P12);(A2,I6,M5,E7,P13);(A2,I6,M5,E7,P14);(A2,I6,M5,E7,P15);(A2,I6,M5,E7,P16);(A2,I6,M5,E7,P17);(A2,I6,M5,E7,P18);(A2,I6,M5,E7,P19);(A2,I6,M5,E7,P20);(A2,I6,M5,E8,P1);(A2,I6,M5,E8,P2);(A2,I6,M5,E8,P3);(A2,I6,M5,E8,P4);(A2,I6,M5,E8,P5);(A2,I6,M5,E8,P6);(A2,I6,M5,E8,P7);(A2,I6,M5,E8,P8);(A2,I6,M5,E8,P9);(A2,I6,M5,E8,P10);(A2,I6,M5,E8,P11);(A2,I6,M5,E8,P12);(A2,I6,M5,E8,P13);(A2,I6,M5,E8,P14);(A2,I6,M5,E8,P15);(A2,I6,M5,E8,P16);(A2,I6,M5,E8,P17);(A2,I6,M5,E8,P18);(A2,I6,M5,E8,P19);(A2,I6,M5,E8,P20);(A2,I6,M5,E9,P1);(A2,I6,M5,E9,P2);(A2,I6,M5,E9,P3);(A2,I6,M5,E9,P4);(A2,I6,M5,E9,P5);(A2,I6,M5,E9,P6);(A2,I6,M5,E9,P7);(A2,I6,M5,E9,P8);(A2,I6,M5,E9,P9);(A2,I6,M5,E9,P10);(A2,I6,M5,E9,P11);(A2,I6,M5,E9,P12);(A2,I6,M5,E9,P13);(A2,I6,M5,E9,P14);(A2,I6,M5,E9,P15);(A2,I6,M5,E9,P16);(A2,I6,M5,E9,P17);(A2,I6,M5,E9,P18);(A2,I6,M5,E9,P19);(A2,I6,M5,E9,P20);(A2,I6,M6,E1,P1);(A2,I6,M6,E1,P2);(A2,I6,M6,E1,P3);(A2,I6,M6,E1,P4);(A2,I6,M6,E1,P5);(A2,I6,M6,E1,P6);(A2,I6,M6,E1,P7);(A2,I6,M6,E1,P8);(A2,I6,M6,E1,P9);(A2,I6,M6,E1,P10);(A2,I6,M6,E1,P11);(A2,I6,M6,E1,P12);(A2,I6,M6,E1,P13);(A2,I6,M6,E1,P14);(A2,I6,M6,E1,P15);(A2,I6,M6,E1,P16);(A2,I6,M6,E1,P17);(A2,I6,M6,E1,P18);(A2,I6,M6,E1,P19);(A2,I6,M6,E1,P20);(A2,I6,M6,E2,P1);(A2,I6,M6,E2,P2);(A2,I6,M6,E2,P3);(A2,I6,M6,E2,P4);(A2,I6,M6,E2,P5);(A2,I6,M6,E2,P6);(A2,I6,M6,E2,P7);(A2,I6,M6,E2,P8);(A2,I6,M6,E2,P9);(A2,I6,M6,E2,P10);(A2,I6,M6,E2,P11);(A2,I6,M6,E2,P12);(A2,I6,M6,E2,P13);(A2,I6,M6,E2,P14);(A2,I6,M6,E2,P15);(A2,I6,M6,E2,P16);(A2,I6,M6,E2,P17);(A2,I6,M6,E2,P18);(A2,I6,M6,E2,P19);(A2,I6,M6,E2,P20);(A2,I6,M6,E3,P1);(A2,I6,M6,E3,P2);(A2,I6,M6,E3,P3);(A2,I6,M6,E3,P4);(A2,I6,M6,E3,P5);(A2,I6,M6,E3,P6);(A2,I6,M6,E3,P7);(A2,I6,M6,E3,P8);(A2,I6,M6,E3,P9);(A2,I6,M6,E3,P10);(A2,I6,M6,E3,P11);(A2,I6,M6,E3,P12);(A2,I6,M6,E3,P13);(A2,I6,M6,E3,P14);(A2,I6,M6,E3,P15);(A2,I6,M6,E3,P16);(A2,I6,M6,E3,P17);(A2,I6,M6,E3,P18);(A2,I6,M6,E3,P19);(A2,I6,M6,E3,P20);(A2,I6,M6,E4,P1);(A2,I6,M6,E4,P2);(A2,I6,M6,E4,P3);(A2,I6,M6,E4,P4);(A2,I6,M6,E4,P5);(A2,I6,M6,E4,P6);(A2,I6,M6,E4,P7);(A2,I6,M6,E4,P8);(A2,I6,M6,E4,P9);(A2,I6,M6,E4,P10);(A2,I6,M6,E4,P11);(A2,I6,M6,E4,P12);(A2,I6,M6,E4,P13);(A2,I6,M6,E4,P14);(A2,I6,M6,E4,P15);(A2,I6,M6,E4,P16);(A2,I6,M6,E4,P17);(A2,I6,M6,E4,P18);(A2,I6,M6,E4,P19);(A2,I6,M6,E4,P20);(A2,I6,M6,E5,P1);(A2,I6,M6,E5,P2);(A2,I6,M6,E5,P3);(A2,I6,M6,E5,P4);(A2,I6,M6,E5,P5);(A2,I6,M6,E5,P6);(A2,I6,M6,E5,P7);(A2,I6,M6,E5,P8);(A2,I6,M6,E5,P9);(A2,I6,M6,E5,P10);(A2,I6,M6,E5,P11);(A2,I6,M6,E5,P12);(A2,I6,M6,E5,P13);(A2,I6,M6,E5,P14);(A2,I6,M6,E5,P15);(A2,I6,M6,E5,P16);(A2,I6,M6,E5,P17);(A2,I6,M6,E5,P18);(A2,I6,M6,E5,P19);(A2,I6,M6,E5,P20);(A2,I6,M6,E6,P1);(A2,I6,M6,E6,P2);(A2,I6,M6,E6,P3);(A2,I6,M6,E6,P4);(A2,I6,M6,E6,P5);(A2,I6,M6,E6,P6);(A2,I6,M6,E6,P7);(A2,I6,M6,E6,P8);(A2,I6,M6,E6,P9);(A2,I6,M6,E6,P10);(A2,I6,M6,E6,P11);(A2,I6,M6,E6,P12);(A2,I6,M6,E6,P13);(A2,I6,M6,E6,P14);(A2,I6,M6,E6,P15);(A2,I6,M6,E6,P16);(A2,I6,M6,E6,P17);(A2,I6,M6,E6,P18);(A2,I6,M6,E6,P19);(A2,I6,M6,E6,P20);(A2,I6,M6,E7,P1);(A2,I6,M6,E7,P2);(A2,I6,M6,E7,P3);(A2,I6,M6,E7,P4);(A2,I6,M6,E7,P5);(A2,I6,M6,E7,P6);(A2,I6,M6,E7,P7);(A2,I6,M6,E7,P8);(A2,I6,M6,E7,P9);(A2,I6,M6,E7,P10);(A2,I6,M6,E7,P11);(A2,I6,M6,E7,P12);(A2,I6,M6,E7,P13);(A2,I6,M6,E7,P14);(A2,I6,M6,E7,P15);(A2,I6,M6,E7,P16);(A2,I6,M6,E7,P17);(A2,I6,M6,E7,P18);(A2,I6,M6,E7,P19);(A2,I6,M6,E7,P20);(A2,I6,M6,E8,P1);(A2,I6,M6,E8,P2);(A2,I6,M6,E8,P3);(A2,I6,M6,E8,P4);(A2,I6,M6,E8,P5);(A2,I6,M6,E8,P6);(A2,I6,M6,E8,P7);(A2,I6,M6,E8,P8);(A2,I6,M6,E8,P9);(A2,I6,M6,E8,P10);(A2,I6,M6,E8,P11);(A2,I6,M6,E8,P12);(A2,I6,M6,E8,P13);(A2,I6,M6,E8,P14);(A2,I6,M6,E8,P15);(A2,I6,M6,E8,P16);(A2,I6,M6,E8,P17);(A2,I6,M6,E8,P18);(A2,I6,M6,E8,P19);(A2,I6,M6,E8,P20);(A2,I6,M6,E9,P1);(A2,I6,M6,E9,P2);(A2,I6,M6,E9,P3);(A2,I6,M6,E9,P4);(A2,I6,M6,E9,P5);(A2,I6,M6,E9,P6);(A2,I6,M6,E9,P7);(A2,I6,M6,E9,P8);(A2,I6,M6,E9,P9);(A2,I6,M6,E9,P10);(A2,I6,M6,E9,P11);(A2,I6,M6,E9,P12);(A2,I6,M6,E9,P13);(A2,I6,M6,E9,P14);(A2,I6,M6,E9,P15);(A2,I6,M6,E9,P16);(A2,I6,M6,E9,P17);(A2,I6,M6,E9,P18);(A2,I6,M6,E9,P19);(A2,I6,M6,E9,P20)A3,I1,M1,E1,P1);(A3,I1,M1,E1,P2);(A3,I1,M1,E1,P3);(A3,I1,M1,E1,P4);(A3,I1,M1,E1,P5);(A3,I1,M1,E1,P6);(A3,I1,M1,E1,P7);(A3,I1,M1,E1,P8);(A3,I1,M1,E1,P9);(A3,I1,M1,E1,P10);(A3,I1,M1,E1,P11);(A3,I1,M1,E1,P12);(A3,I1,M1,E1,P13);(A3,I1,M1,E1,P14);(A3,I1,M1,E1,P15);(A3,I1,M1,E1,P16);(A3,I1,M1,E1,P17);(A3,I1,M1,E1,P18);(A3,I1,M1,E1,P19);(A3,I1,M1,E1,P20);(A3,I1,M1,E2,P1);(A3,I1,M1,E2,P2);(A3,I1,M1,E2,P3);(A3,I1,M1,E2,P4);(A3,I1,M1,E2,P5);(A3,I1,M1,E2,P6);(A3,I1,M1,E2,P7);(A3,I1,M1,E2,P8);(A3,I1,M1,E2,P9);(A3,I1,M1,E2,P10);(A3,I1,M1,E2,P11);(A3,I1,M1,E2,P12);(A3,I1,M1,E2,P13);(A3,I1,M1,E2,P14);(A3,I1,M1,E2,P15);(A3,I1,M1,E2,P16);(A3,I1,M1,E2,P17);(A3,I1,M1,E2,P18);(A3,I1,M1,E2,P19);(A3,I1,M1,E2,P20);(A3,I1,M1,E3,P1);(A3,I1,M1,E3,P2);(A3,I1,M1,E3,P3);(A3,I1,M1,E3,P4);(A3,I1,M1,E3,P5);(A3,I1,M1,E3,P6);(A3,I1,M1,E3,P7);(A3,I1,M1,E3,P8);(A3,I1,M1,E3,P9);(A3,I1,M1,E3,P10);(A3,I1,M1,E3,P11);(A3,I1,M1,E3,P12);(A3,I1,M1,E3,P13);(A3,I1,M1,E3,P14);(A3,I1,M1,E3,P15);(A3,I1,M1,E3,P16);(A3,I1,M1,E3,P17);(A3,I1,M1,E3,P18);(A3,I1,M1,E3,P19);(A3,I1,M1,E3,P20);(A3,I1,M1,E4,P1);(A3,I1,M1,E4,P2);(A3,I1,M1,E4,P3);(A3,I1,M1,E4,P4);(A3,I1,M1,E4,P5);(A3,I1,M1,E4,P6);(A3,I1,M1,E4,P7);(A3,I1,M1,E4,P8);(A3,I1,M1,E4,P9);(A3,I1,M1,E4,P10);(A3,I1,M1,E4,P11);(A3,I1,M1,E4,P12);(A3,I1,M1,E4,P13);(A3,I1,M1,E4,P14);(A3,I1,M1,E4,P15);(A3,I1,M1,E4,P16);(A3,I1,M1,E4,P17);(A3,I1,M1,E4,P18);(A3,I1,M1,E4,P19);(A3,I1,M1,E4,P20);(A3,I1,M1,E5,P1);(A3,I1,M1,E5,P2);(A3,I1,M1,E5,P3);(A3,I1,M1,E5,P4);(A3,I1,M1,E5,P5);(A3,I1,M1,E5,P6);(A3,I1,M1,E5,P7);(A3,I1,M1,E5,P8);(A3,I1,M1,E5,P9);(A3,I1,M1,E5,P10);(A3,I1,M1,E5,P11);(A3,I1,M1,E5,P12);(A3,I1,M1,E5,P13);(A3,I1,M1,E5,P14);(A3,I1,M1,E5,P15);(A3,I1,M1,E5,P16);(A3,I1,M1,E5,P17);(A3,I1,M1,E5,P18);(A3,I1,M1,E5,P19);(A3,I1,M1,E5,P20);(A3,I1,M1,E6,P1);(A3,I1,M1,E6,P2);(A3,I1,M1,E6,P3);(A3,I1,M1,E6,P4);(A3,I1,M1,E6,P5);(A3,I1,M1,E6,P6);(A3,I1,M1,E6,P7);(A3,I1,M1,E6,P8);(A3,I1,M1,E6,P9);(A3,I1,M1,E6,P10);(A3,I1,M1,E6,P11);(A3,I1,M1,E6,P12);(A3,I1,M1,E6,P13);(A3,I1,M1,E6,P14);(A3,I1,M1,E6,P15);(A3,I1,M1,E6,P16);(A3,I1,M1,E6,P17);(A3,I1,M1,E6,P18);(A3,I1,M1,E6,P19);(A3,I1,M1,E6,P20);(A3,I1,M1,E7,P1);(A3,I1,M1,E7,P2);(A3,I1,M1,E7,P3);(A3,I1,M1,E7,P4);(A3,I1,M1,E7,P5);(A3,I1,M1,E7,P6);(A3,I1,M1,E7,P7);(A3,I1,M1,E7,P8);(A3,I1,M1,E7,P9);(A3,I1,M1,E7,P10);(A3,I1,M1,E7,P11);(A3,I1,M1,E7,P12);(A3,I1,M1,E7,P13);(A3,I1,M1,E7,P14);(A3,I1,M1,E7,P15);(A3,I1,M1,E7,P16);(A3,I1,M1,E7,P17);(A3,I1,M1,E7,P18);(A3,I1,M1,E7,P19);(A3,I1,M1,E7,P20);(A3,I1,M1,E8,P1);(A3,I1,M1,E8,P2);(A3,I1,M1,E8,P3);(A3,I1,M1,E8,P4);(A3,I1,M1,E8,P5);(A3,I1,M1,E8,P6);(A3,I1,M1,E8,P7);(A3,I1,M1,E8,P8);(A3,I1,M1,E8,P9);(A3,I1,M1,E8,P10);(A3,I1,M1,E8,P11);(A3,I1,M1,E8,P12);(A3,I1,M1,E8,P13);(A3,I1,M1,E8,P14);(A3,I1,M1,E8,P15);(A3,I1,M1,E8,P16);(A3,I1,M1,E8,P17);(A3,I1,M1,E8,P18);(A3,I1,M1,E8,P19);(A3,I1,M1,E8,P20);(A3,I1,M1,E9,P1);(A3,I1,M1,E9,P2);(A3,I1,M1,E9,P3);(A3,I1,M1,E9,P4);(A3,I1,M1,E9,P5);(A3,I1,M1,E9,P6);(A3,I1,M1,E9,P7);(A3,I1,M1,E9,P8);(A3,I1,M1,E9,P9);(A3,I1,M1,E9,P10);(A3,I1,M1,E9,P11);(A3,I1,M1,E9,P12);(A3,I1,M1,E9,P13);(A3,I1,M1,E9,P14);(A3,I1,M1,E9,P15);(A3,I1,M1,E9,P16);(A3,I1,M1,E9,P17);(A3,I1,M1,E9,P18);(A3,I1,M1,E9,P19);(A3,I1,M1,E9,P20);(A3,I1,M2,E1,P1);(A3,I1,M2,E1,P2);(A3,I1,M2,E1,P3);(A3,I1,M2,E1,P4);(A3,I1,M2,E1,P5);(A3,I1,M2,E1,P6);(A3,I1,M2,E1,P7);(A3,I1,M2,E1,P8);(A3,I1,M2,E1,P9);(A3,I1,M2,E1,P10);(A3,I1,M2,E1,P11);(A3,I1,M2,E1,P12);(A3,I1,M2,E1,P13);(A3,I1,M2,E1,P14);(A3,I1,M2,E1,P15);(A3,I1,M2,E1,P16);(A3,I1,M2,E1,P17);(A3,I1,M2,E1,P18);(A3,I1,M2,E1,P19);(A3,I1,M2,E1,P20);(A3,I1,M2,E2,P1);(A3,I1,M2,E2,P2);(A3,I1,M2,E2,P3);(A3,I1,M2,E2,P4);(A3,I1,M2,E2,P5);(A3,I1,M2,E2,P6);(A3,I1,M2,E2,P7);(A3,I1,M2,E2,P8);(A3,I1,M2,E2,P9);(A3,I1,M2,E2,P10);(A3,I1,M2,E2,P11);(A3,I1,M2,E2,P12);(A3,I1,M2,E2,P13);(A3,I1,M2,E2,P14);(A3,I1,M2,E2,P15);(A3,I1,M2,E2,P16);(A3,I1,M2,E2,P17);(A3,I1,M2,E2,P18);(A3,I1,M2,E2,P19);(A3,I1,M2,E2,P20);(A3,I1,M2,E3,P1);(A3,I1,M2,E3,P2);(A3,I1,M2,E3,P3);(A3,I1,M2,E3,P4);(A3,I1,M2,E3,P5);(A3,I1,M2,E3,P6);(A3,I1,M2,E3,P7);(A3,I1,M2,E3,P8);(A3,I1,M2,E3,P9);(A3,I1,M2,E3,P10);(A3,I1,M2,E3,P11);(A3,I1,M2,E3,P12);(A3,I1,M2,E3,P13);(A3,I1,M2,E3,P14);(A3,I1,M2,E3,P15);(A3,I1,M2,E3,P16);(A3,I1,M2,E3,P17);(A3,I1,M2,E3,P18);(A3,I1,M2,E3,P19);(A3,I1,M2,E3,P20);(A3,I1,M2,E4,P1);(A3,I1,M2,E4,P2);(A3,I1,M2,E4,P3);(A3,I1,M2,E4,P4);(A3,I1,M2,E4,P5);(A3,I1,M2,E4,P6);(A3,I1,M2,E4,P7);(A3,I1,M2,E4,P8);(A3,I1,M2,E4,P9);(A3,I1,M2,E4,P10);(A3,I1,M2,E4,P11);(A3,I1,M2,E4,P12);(A3,I1,M2,E4,P13);(A3,I1,M2,E4,P14);(A3,I1,M2,E4,P15);(A3,I1,M2,E4,P16);(A3,I1,M2,E4,P17);(A3,I1,M2,E4,P18);(A3,I1,M2,E4,P19);(A3,I1,M2,E4,P20);(A3,I1,M2,E5,P1);(A3,I1,M2,E5,P2);(A3,I1,M2,E5,P3);(A3,I1,M2,E5,P4);(A3,I1,M2,E5,P5);(A3,I1,M2,E5,P6);(A3,I1,M2,E5,P7);(A3,I1,M2,E5,P8);(A3,I1,M2,E5,P9);(A3,I1,M2,E5,P10);(A3,I1,M2,E5,P11);(A3,I1,M2,E5,P12);(A3,I1,M2,E5,P13);(A3,I1,M2,E5,P14);(A3,I1,M2,E5,P15);(A3,I1,M2,E5,P16);(A3,I1,M2,E5,P17);(A3,I1,M2,E5,P18);(A3,I1,M2,E5,P19);(A3,I1,M2,E5,P20);(A3,I1,M2,E6,P1);(A3,I1,M2,E6,P2);(A3,I1,M2,E6,P3);(A3,I1,M2,E6,P4);(A3,I1,M2,E6,P5);(A3,I1,M2,E6,P6);(A3,I1,M2,E6,P7);(A3,I1,M2,E6,P8);(A3,I1,M2,E6,P9);(A3,I1,M2,E6,P10);(A3,I1,M2,E6,P11);(A3,I1,M2,E6,P12);(A3,I1,M2,E6,P13);(A3,I1,M2,E6,P14);(A3,I1,M2,E6,P15);(A3,I1,M2,E6,P16);(A3,I1,M2,E6,P17);(A3,I1,M2,E6,P18);(A3,I1,M2,E6,P19);(A3,I1,M2,E6,P20);(A3,I1,M2,E7,P1);(A3,I1,M2,E7,P2);(A3,I1,M2,E7,P3);(A3,I1,M2,E7,P4);(A3,I1,M2,E7,P5);(A3,I1,M2,E7,P6);(A3,I1,M2,E7,P7);(A3,I1,M2,E7,P8);(A3,I1,M2,E7,P9);(A3,I1,M2,E7,P10);(A3,I1,M2,E7,P11);(A3,I1,M2,E7,P12);(A3,I1,M2,E7,P13);(A3,I1,M2,E7,P14);(A3,I1,M2,E7,P15);(A3,I1,M2,E7,P16);(A3,I1,M2,E7,P17);(A3,I1,M2,E7,P18);(A3,I1,M2,E7,P19);(A3,I1,M2,E7,P20);(A3,I1,M2,E8,P1);(A3,I1,M2,E8,P2);(A3,I1,M2,E8,P3);(A3,I1,M2,E8,P4);(A3,I1,M2,E8,P5);(A3,I1,M2,E8,P6);(A3,I1,M2,E8,P7);(A3,I1,M2,E8,P8);(A3,I1,M2,E8,P9);(A3,I1,M2,E8,P10);(A3,I1,M2,E8,P11);(A3,I1,M2,E8,P12);(A3,I1,M2,E8,P13);(A3,I1,M2,E8,P14);(A3,I1,M2,E8,P15);(A3,I1,M2,E8,P16);(A3,I1,M2,E8,P17);(A3,I1,M2,E8,P18);(A3,I1,M2,E8,P19);(A3,I1,M2,E8,P20);(A3,I1,M2,E9,P1);(A3,I1,M2,E9,P2);(A3,I1,M2,E9,P3);(A3,I1,M2,E9,P4);(A3,I1,M2,E9,P5);(A3,I1,M2,E9,P6);(A3,I1,M2,E9,P7);(A3,I1,M2,E9,P8);(A3,I1,M2,E9,P9);(A3,I1,M2,E9,P10);(A3,I1,M2,E9,P11);(A3,I1,M2,E9,P12);(A3,I1,M2,E9,P13);(A3,I1,M2,E9,P14);(A3,I1,M2,E9,P15);(A3,I1,M2,E9,P16);(A3,I1,M2,E9,P17);(A3,I1,M2,E9,P18);(A3,I1,M2,E9,P19);(A3,I1,M2,E9,P20);(A3,I1,M3,E1,P1);(A3,I1,M3,E1,P2);(A3,I1,M3,E1,P3);(A3,I1,M3,E1,P4);(A3,I1,M3,E1,P5);(A3,I1,M3,E1,P6);(A3,I1,M3,E1,P7);(A3,I1,M3,E1,P8);(A3,I1,M3,E1,P9);(A3,I1,M3,E1,P10);(A3,I1,M3,E1,P11);(A3,I1,M3,E1,P12);(A3,I1,M3,E1,P13);(A3,I1,M3,E1,P14);(A3,I1,M3,E1,P15);(A3,I1,M3,E1,P16);(A3,I1,M3,E1,P17);(A3,I1,M3,E1,P18);(A3,I1,M3,E1,P19);(A3,I1,M3,E1,P20);(A3,I1,M3,E2,P1);(A3,I1,M3,E2,P2);(A3,I1,M3,E2,P3);(A3,I1,M3,E2,P4);(A3,I1,M3,E2,P5);(A3,I1,M3,E2,P6);(A3,I1,M3,E2,P7);(A3,I1,M3,E2,P8);(A3,I1,M3,E2,P9);(A3,I1,M3,E2,P10);(A3,I1,M3,E2,P11);(A3,I1,M3,E2,P12);(A3,I1,M3,E2,P13);(A3,I1,M3,E2,P14);(A3,I1,M3,E2,P15);(A3,I1,M3,E2,P16);(A3,I1,M3,E2,P17);(A3,I1,M3,E2,P18);(A3,I1,M3,E2,P19);(A3,I1,M3,E2,P20);(A3,I1,M3,E3,P1);(A3,I1,M3,E3,P2);(A3,I1,M3,E3,P3);(A3,I1,M3,E3,P4);(A3,I1,M3,E3,P5);(A3,I1,M3,E3,P6);(A3,I1,M3,E3,P7);(A3,I1,M3,E3,P8);(A3,I1,M3,E3,P9);(A3,I1,M3,E3,P10);(A3,I1,M3,E3,P11);(A3,I1,M3,E3,P12);(A3,I1,M3,E3,P13);(A3,I1,M3,E3,P14);(A3,I1,M3,E3,P15);(A3,I1,M3,E3,P16);(A3,I1,M3,E3,P17);(A3,I1,M3,E3,P18);(A3,I1,M3,E3,P19);(A3,I1,M3,E3,P20);(A3,I1,M3,E4,P1);(A3,I1,M3,E4,P2);(A3,I1,M3,E4,P3);(A3,I1,M3,E4,P4);(A3,I1,M3,E4,P5);(A3,I1,M3,E4,P6);(A3,I1,M3,E4,P7);(A3,I1,M3,E4,P8);(A3,I1,M3,E4,P9);(A3,I1,M3,E4,P10);(A3,I1,M3,E4,P11);(A3,I1,M3,E4,P12);(A3,I1,M3,E4,P13);(A3,I1,M3,E4,P14);(A3,I1,M3,E4,P15);(A3,I1,M3,E4,P16);(A3,I1,M3,E4,P17);(A3,I1,M3,E4,P18);(A3,I1,M3,E4,P19);(A3,I1,M3,E4,P20);(A3,I1,M3,E5,P1);(A3,I1,M3,E5,P2);(A3,I1,M3,E5,P3);(A3,I1,M3,E5,P4);(A3,I1,M3,E5,P5);(A3,I1,M3,E5,P6);(A3,I1,M3,E5,P7);(A3,I1,M3,E5,P8);(A3,I1,M3,E5,P9);(A3,I1,M3,E5,P10);(A3,I1,M3,E5,P11);(A3,I1,M3,E5,P12);(A3,I1,M3,E5,P13);(A3,I1,M3,E5,P14);(A3,I1,M3,E5,P15);(A3,I1,M3,E5,P16);(A3,I1,M3,E5,P17);(A3,I1,M3,E5,P18);(A3,I1,M3,E5,P19);(A3,I1,M3,E5,P20);(A3,I1,M3,E6,P1);(A3,I1,M3,E6,P2);(A3,I1,M3,E6,P3);(A3,I1,M3,E6,P4);(A3,I1,M3,E6,P5);(A3,I1,M3,E6,P6);(A3,I1,M3,E6,P7);(A3,I1,M3,E6,P8);(A3,I1,M3,E6,P9);(A3,I1,M3,E6,P10);(A3,I1,M3,E6,P11);(A3,I1,M3,E6,P12);(A3,I1,M3,E6,P13);(A3,I1,M3,E6,P14);(A3,I1,M3,E6,P15);(A3,I1,M3,E6,P16);(A3,I1,M3,E6,P17);(A3,I1,M3,E6,P18);(A3,I1,M3,E6,P19);(A3,I1,M3,E6,P20);(A3,I1,M3,E7,P1);(A3,I1,M3,E7,P2);(A3,I1,M3,E7,P3);(A3,I1,M3,E7,P4);(A3,I1,M3,E7,P5);(A3,I1,M3,E7,P6);(A3,I1,M3,E7,P7);(A3,I1,M3,E7,P8);(A3,I1,M3,E7,P9);(A3,I1,M3,E7,P10);(A3,I1,M3,E7,P11);(A3,I1,M3,E7,P12);(A3,I1,M3,E7,P13);(A3,I1,M3,E7,P14);(A3,I1,M3,E7,P15);(A3,I1,M3,E7,P16);(A3,I1,M3,E7,P17);(A3,I1,M3,E7,P18);(A3,I1,M3,E7,P19);(A3,I1,M3,E7,P20);(A3,I1,M3,E8,P1);(A3,I1,M3,E8,P2);(A3,I1,M3,E8,P3);(A3,I1,M3,E8,P4);(A3,I1,M3,E8,P5);(A3,I1,M3,E8,P6);(A3,I1,M3,E8,P7);(A3,I1,M3,E8,P8);(A3,I1,M3,E8,P9);(A3,I1,M3,E8,P10);(A3,I1,M3,E8,P11);(A3,I1,M3,E8,P12);(A3,I1,M3,E8,P13);(A3,I1,M3,E8,P14);(A3,I1,M3,E8,P15);(A3,I1,M3,E8,P16);(A3,I1,M3,E8,P17);(A3,I1,M3,E8,P18);(A3,I1,M3,E8,P19);(A3,I1,M3,E8,P20);(A3,I1,M3,E9,P1);(A3,I1,M3,E9,P2);(A3,I1,M3,E9,P3);(A3,I1,M3,E9,P4);(A3,I1,M3,E9,P5);(A3,I1,M3,E9,P6);(A3,I1,M3,E9,P7);(A3,I1,M3,E9,P8);(A3,I1,M3,E9,P9);(A3,I1,M3,E9,P10);(A3,I1,M3,E9,P11);(A3,I1,M3,E9,P12);(A3,I1,M3,E9,P13);(A3,I1,M3,E9,P14);(A3,I1,M3,E9,P15);(A3,I1,M3,E9,P16);(A3,I1,M3,E9,P17);(A3,I1,M3,E9,P18);(A3,I1,M3,E9,P19);(A3,I1,M3,E9,P20);(A3,I1,M4,E1,P1);(A3,I1,M4,E1,P2);(A3,I1,M4,E1,P3);(A3,I1,M4,E1,P4);(A3,I1,M4,E1,P5);(A3,I1,M4,E1,P6);(A3,I1,M4,E1,P7);(A3,I1,M4,E1,P8);(A3,I1,M4,E1,P9);(A3,I1,M4,E1,P10);(A3,I1,M4,E1,P11);(A3,I1,M4,E1,P12);(A3,I1,M4,E1,P13);(A3,I1,M4,E1,P14);(A3,I1,M4,E1,P15);(A3,I1,M4,E1,P16);(A3,I1,M4,E1,P17);(A3,I1,M4,E1,P18);(A3,I1,M4,E1,P19);(A3,I1,M4,E1,P20);(A3,I1,M4,E2,P1);(A3,I1,M4,E2,P2);(A3,I1,M4,E2,P3);(A3,I1,M4,E2,P4);(A3,I1,M4,E2,P5);(A3,I1,M4,E2,P6);(A3,I1,M4,E2,P7);(A3,I1,M4,E2,P8);(A3,I1,M4,E2,P9);(A3,I1,M4,E2,P10);(A3,I1,M4,E2,P11);(A3,I1,M4,E2,P12);(A3,I1,M4,E2,P13);(A3,I1,M4,E2,P14);(A3,I1,M4,E2,P15);(A3,I1,M4,E2,P16);(A3,I1,M4,E2,P17);(A3,I1,M4,E2,P18);(A3,I1,M4,E2,P19);(A3,I1,M4,E2,P20);(A3,I1,M4,E3,P1);(A3,I1,M4,E3,P2);(A3,I1,M4,E3,P3);(A3,I1,M4,E3,P4);(A3,I1,M4,E3,P5);(A3,I1,M4,E3,P6);(A3,I1,M4,E3,P7);(A3,I1,M4,E3,P8);(A3,I1,M4,E3,P9);(A3,I1,M4,E3,P10);(A3,I1,M4,E3,P11);(A3,I1,M4,E3,P12);(A3,I1,M4,E3,P13);(A3,I1,M4,E3,P14);(A3,I1,M4,E3,P15);(A3,I1,M4,E3,P16);(A3,I1,M4,E3,P17);(A3,I1,M4,E3,P18);(A3,I1,M4,E3,P19);(A3,I1,M4,E3,P20);(A3,I1,M4,E4,P1);(A3,I1,M4,E4,P2);(A3,I1,M4,E4,P3);(A3,I1,M4,E4,P4);(A3,I1,M4,E4,P5);(A3,I1,M4,E4,P6);(A3,I1,M4,E4,P7);(A3,I1,M4,E4,P8);(A3,I1,M4,E4,P9);(A3,I1,M4,E4,P10);(A3,I1,M4,E4,P11);(A3,I1,M4,E4,P12);(A3,I1,M4,E4,P13);(A3,I1,M4,E4,P14);(A3,I1,M4,E4,P15);(A3,I1,M4,E4,P16);(A3,I1,M4,E4,P17);(A3,I1,M4,E4,P18);(A3,I1,M4,E4,P19);(A3,I1,M4,E4,P20);(A3,I1,M4,E5,P1);(A3,I1,M4,E5,P2);(A3,I1,M4,E5,P3);(A3,I1,M4,E5,P4);(A3,I1,M4,E5,P5);(A3,I1,M4,E5,P6);(A3,I1,M4,E5,P7);(A3,I1,M4,E5,P8);(A3,I1,M4,E5,P9);(A3,I1,M4,E5,P10);(A3,I1,M4,E5,P11);(A3,I1,M4,E5,P12);(A3,I1,M4,E5,P13);(A3,I1,M4,E5,P14);(A3,I1,M4,E5,P15);(A3,I1,M4,E5,P16);(A3,I1,M4,E5,P17);(A3,I1,M4,E5,P18);(A3,I1,M4,E5,P19);(A3,I1,M4,E5,P20);(A3,I1,M4,E6,P1);(A3,I1,M4,E6,P2);(A3,I1,M4,E6,P3);(A3,I1,M4,E6,P4);(A3,I1,M4,E6,P5);(A3,I1,M4,E6,P6);(A3,I1,M4,E6,P7);(A3,I1,M4,E6,P8);(A3,I1,M4,E6,P9);(A3,I1,M4,E6,P10);(A3,I1,M4,E6,P11);(A3,I1,M4,E6,P12);(A3,I1,M4,E6,P13);(A3,I1,M4,E6,P14);(A3,I1,M4,E6,P15);(A3,I1,M4,E6,P16);(A3,I1,M4,E6,P17);(A3,I1,M4,E6,P18);(A3,I1,M4,E6,P19);(A3,I1,M4,E6,P20);(A3,I1,M4,E7,P1);(A3,I1,M4,E7,P2);(A3,I1,M4,E7,P3);(A3,I1,M4,E7,P4);(A3,I1,M4,E7,P5);(A3,I1,M4,E7,P6);(A3,I1,M4,E7,P7);(A3,I1,M4,E7,P8);(A3,I1,M4,E7,P9);(A3,I1,M4,E7,P10);(A3,I1,M4,E7,P11);(A3,I1,M4,E7,P12);(A3,I1,M4,E7,P13);(A3,I1,M4,E7,P14);(A3,I1,M4,E7,P15);(A3,I1,M4,E7,P16);(A3,I1,M4,E7,P17);(A3,I1,M4,E7,P18);(A3,I1,M4,E7,P19);(A3,I1,M4,E7,P20);(A3,I1,M4,E8,P1);(A3,I1,M4,E8,P2);(A3,I1,M4,E8,P3);(A3,I1,M4,E8,P4);(A3,I1,M4,E8,P5);(A3,I1,M4,E8,P6);(A3,I1,M4,E8,P7);(A3,I1,M4,E8,P8);(A3,I1,M4,E8,P9);(A3,I1,M4,E8,P10);(A3,I1,M4,E8,P11);(A3,I1,M4,E8,P12);(A3,I1,M4,E8,P13);(A3,I1,M4,E8,P14);(A3,I1,M4,E8,P15);(A3,I1,M4,E8,P16);(A3,I1,M4,E8,P17);(A3,I1,M4,E8,P18);(A3,I1,M4,E8,P19);(A3,I1,M4,E8,P20);(A3,I1,M4,E9,P1);(A3,I1,M4,E9,P2);(A3,I1,M4,E9,P3);(A3,I1,M4,E9,P4);(A3,I1,M4,E9,P5);(A3,I1,M4,E9,P6);(A3,I1,M4,E9,P7);(A3,I1,M4,E9,P8);(A3,I1,M4,E9,P9);(A3,I1,M4,E9,P10);(A3,I1,M4,E9,P11);(A3,I1,M4,E9,P12);(A3,I1,M4,E9,P13);(A3,I1,M4,E9,P14);(A3,I1,M4,E9,P15);(A3,I1,M4,E9,P16);(A3,I1,M4,E9,P17);(A3,I1,M4,E9,P18);(A3,I1,M4,E9,P19);(A3,I1,M4,E9,P20);(A3,I1,M5,E1,P1);(A3,I1,M5,E1,P2);(A3,I1,M5,E1,P3);(A3,I1,M5,E1,P4);(A3,I1,M5,E1,P5);(A3,I1,M5,E1,P6);(A3,I1,M5,E1,P7);(A3,I1,M5,E1,P8);(A3,I1,M5,E1,P9);(A3,I1,M5,E1,P10);(A3,I1,M5,E1,P11);(A3,I1,M5,E1,P12);(A3,I1,M5,E1,P13);(A3,I1,M5,E1,P14);(A3,I1,M5,E1,P15);(A3,I1,M5,E1,P16);(A3,I1,M5,E1,P17);(A3,I1,M5,E1,P18);(A3,I1,M5,E1,P19);(A3,I1,M5,E1,P20);(A3,I1,M5,E2,P1);(A3,I1,M5,E2,P2);(A3,I1,M5,E2,P3);(A3,I1,M5,E2,P4);(A3,I1,M5,E2,P5);(A3,I1,M5,E2,P6);(A3,I1,M5,E2,P7);(A3,I1,M5,E2,P8);(A3,I1,M5,E2,P9);(A3,I1,M5,E2,P10);(A3,I1,M5,E2,P11);(A3,I1,M5,E2,P12);(A3,I1,M5,E2,P13);(A3,I1,M5,E2,P14);(A3,I1,M5,E2,P15);(A3,I1,M5,E2,P16);(A3,I1,M5,E2,P17);(A3,I1,M5,E2,P18);(A3,I1,M5,E2,P19);(A3,I1,M5,E2,P20);(A3,I1,M5,E3,P1);(A3,I1,M5,E3,P2);(A3,I1,M5,E3,P3);(A3,I1,M5,E3,P4);(A3,I1,M5,E3,P5);(A3,I1,M5,E3,P6);(A3,I1,M5,E3,P7);(A3,I1,M5,E3,P8);(A3,I1,M5,E3,P9);(A3,I1,M5,E3,P10);(A3,I1,M5,E3,P11);(A3,I1,M5,E3,P12);(A3,I1,M5,E3,P13);(A3,I1,M5,E3,P14);(A3,I1,M5,E3,P15);(A3,I1,M5,E3,P16);(A3,I1,M5,E3,P17);(A3,I1,M5,E3,P18);(A3,I1,M5,E3,P19);(A3,I1,M5,E3,P20);(A3,I1,M5,E4,P1);(A3,I1,M5,E4,P2);(A3,I1,M5,E4,P3);(A3,I1,M5,E4,P4);(A3,I1,M5,E4,P5);(A3,I1,M5,E4,P6);(A3,I1,M5,E4,P7);(A3,I1,M5,E4,P8);(A3,I1,M5,E4,P9);(A3,I1,M5,E4,P10);(A3,I1,M5,E4,P11);(A3,I1,M5,E4,P12);(A3,I1,M5,E4,P13);(A3,I1,M5,E4,P14);(A3,I1,M5,E4,P15);(A3,I1,M5,E4,P16);(A3,I1,M5,E4,P17);(A3,I1,M5,E4,P18);(A3,I1,M5,E4,P19);(A3,I1,M5,E4,P20);(A3,I1,M5,E5,P1);(A3,I1,M5,E5,P2);(A3,I1,M5,E5,P3);(A3,I1,M5,E5,P4);(A3,I1,M5,E5,P5);(A3,I1,M5,E5,P6);(A3,I1,M5,E5,P7);(A3,I1,M5,E5,P8);(A3,I1,M5,E5,P9);(A3,I1,M5,E5,P10);(A3,I1,M5,E5,P11);(A3,I1,M5,E5,P12);(A3,I1,M5,E5,P13);(A3,I1,M5,E5,P14);(A3,I1,M5,E5,P15);(A3,I1,M5,E5,P16);(A3,I1,M5,E5,P17);(A3,I1,M5,E5,P18);(A3,I1,M5,E5,P19);(A3,I1,M5,E5,P20);(A3,I1,M5,E6,P1);(A3,I1,M5,E6,P2);(A3,I1,M5,E6,P3);(A3,I1,M5,E6,P4);(A3,I1,M5,E6,P5);(A3,I1,M5,E6,P6);(A3,I1,M5,E6,P7);(A3,I1,M5,E6,P8);(A3,I1,M5,E6,P9);(A3,I1,M5,E6,P10);(A3,I1,M5,E6,P11);(A3,I1,M5,E6,P12);(A3,I1,M5,E6,P13);(A3,I1,M5,E6,P14);(A3,I1,M5,E6,P15);(A3,I1,M5,E6,P16);(A3,I1,M5,E6,P17);(A3,I1,M5,E6,P18);(A3,I1,M5,E6,P19);(A3,I1,M5,E6,P20);(A3,I1,M5,E7,P1);(A3,I1,M5,E7,P2);(A3,I1,M5,E7,P3);(A3,I1,M5,E7,P4);(A3,I1,M5,E7,P5);(A3,I1,M5,E7,P6);(A3,I1,M5,E7,P7);(A3,I1,M5,E7,P8);(A3,I1,M5,E7,P9);(A3,I1,M5,E7,P10);(A3,I1,M5,E7,P11);(A3,I1,M5,E7,P12);(A3,I1,M5,E7,P13);(A3,I1,M5,E7,P14);(A3,I1,M5,E7,P15);(A3,I1,M5,E7,P16);(A3,I1,M5,E7,P17);(A3,I1,M5,E7,P18);(A3,I1,M5,E7,P19);(A3,I1,M5,E7,P20);(A3,I1,M5,E8,P1);(A3,I1,M5,E8,P2);(A3,I1,M5,E8,P3);(A3,I1,M5,E8,P4);(A3,I1,M5,E8,P5);(A3,I1,M5,E8,P6);(A3,I1,M5,E8,P7);(A3,I1,M5,E8,P8);(A3,I1,M5,E8,P9);(A3,I1,M5,E8,P10);(A3,I1,M5,E8,P11);(A3,I1,M5,E8,P12);(A3,I1,M5,E8,P13);(A3,I1,M5,E8,P14);(A3,I1,M5,E8,P15);(A3,I1,M5,E8,P16);(A3,I1,M5,E8,P17);(A3,I1,M5,E8,P18);(A3,I1,M5,E8,P19);(A3,I1,M5,E8,P20);(A3,I1,M5,E9,P1);(A3,I1,M5,E9,P2);(A3,I1,M5,E9,P3);(A3,I1,M5,E9,P4);(A3,I1,M5,E9,P5);(A3,I1,M5,E9,P6);(A3,I1,M5,E9,P7);(A3,I1,M5,E9,P8);(A3,I1,M5,E9,P9);(A3,I1,M5,E9,P10);(A3,I1,M5,E9,P11);(A3,I1,M5,E9,P12);(A3,I1,M5,E9,P13);(A3,I1,M5,E9,P14);(A3,I1,M5,E9,P15);(A3,I1,M5,E9,P16);(A3,I1,M5,E9,P17);(A3,I1,M5,E9,P18);(A3,I1,M5,E9,P19);(A3,I1,M5,E9,P20);(A3,I1,M6,E1,P1);(A3,I1,M6,E1,P2);(A3,I1,M6,E1,P3);(A3,I1,M6,E1,P4);(A3,I1,M6,E1,P5);(A3,I1,M6,E1,P6);(A3,I1,M6,E1,P7);(A3,I1,M6,E1,P8);(A3,I1,M6,E1,P9);(A3,I1,M6,E1,P10);(A3,I1,M6,E1,P11);(A3,I1,M6,E1,P12);(A3,I1,M6,E1,P13);(A3,I1,M6,E1,P14);(A3,I1,M6,E1,P15);(A3,I1,M6,E1,P16);(A3,I1,M6,E1,P17);(A3,I1,M6,E1,P18);(A3,I1,M6,E1,P19);(A3,I1,M6,E1,P20);(A3,I1,M6,E2,P1);(A3,I1,M6,E2,P2);(A3,I1,M6,E2,P3);(A3,I1,M6,E2,P4);(A3,I1,M6,E2,P5);(A3,I1,M6,E2,P6);(A3,I1,M6,E2,P7);(A3,I1,M6,E2,P8);(A3,I1,M6,E2,P9);(A3,I1,M6,E2,P10);(A3,I1,M6,E2,P11);(A3,I1,M6,E2,P12);(A3,I1,M6,E2,P13);(A3,I1,M6,E2,P14);(A3,I1,M6,E2,P15);(A3,I1,M6,E2,P16);(A3,I1,M6,E2,P17);(A3,I1,M6,E2,P18);(A3,I1,M6,E2,P19);(A3,I1,M6,E2,P20);(A3,I1,M6,E3,P1);(A3,I1,M6,E3,P2);(A3,I1,M6,E3,P3);(A3,I1,M6,E3,P4);(A3,I1,M6,E3,P5);(A3,I1,M6,E3,P6);(A3,I1,M6,E3,P7);(A3,I1,M6,E3,P8);(A3,I1,M6,E3,P9);(A3,I1,M6,E3,P10);(A3,I1,M6,E3,P11);(A3,I1,M6,E3,P12);(A3,I1,M6,E3,P13);(A3,I1,M6,E3,P14);(A3,I1,M6,E3,P15);(A3,I1,M6,E3,P16);(A3,I1,M6,E3,P17);(A3,I1,M6,E3,P18);(A3,I1,M6,E3,P19);(A3,I1,M6,E3,P20);(A3,I1,M6,E4,P1);(A3,I1,M6,E4,P2);(A3,I1,M6,E4,P3);(A3,I1,M6,E4,P4);(A3,I1,M6,E4,P5);(A3,I1,M6,E4,P6);(A3,I1,M6,E4,P7);(A3,I1,M6,E4,P8);(A3,I1,M6,E4,P9);(A3,I1,M6,E4,P10);(A3,I1,M6,E4,P11);(A3,I1,M6,E4,P12);(A3,I1,M6,E4,P13);(A3,I1,M6,E4,P14);(A3,I1,M6,E4,P15);(A3,I1,M6,E4,P16);(A3,I1,M6,E4,P17);(A3,I1,M6,E4,P18);(A3,I1,M6,E4,P19);(A3,I1,M6,E4,P20);(A3,I1,M6,E5,P1);(A3,I1,M6,E5,P2);(A3,I1,M6,E5,P3);(A3,I1,M6,E5,P4);(A3,I1,M6,E5,P5);(A3,I1,M6,E5,P6);(A3,I1,M6,E5,P7);(A3,I1,M6,E5,P8);(A3,I1,M6,E5,P9);(A3,I1,M6,E5,P10);(A3,I1,M6,E5,P11);(A3,I1,M6,E5,P12);(A3,I1,M6,E5,P13);(A3,I1,M6,E5,P14);(A3,I1,M6,E5,P15);(A3,I1,M6,E5,P16);(A3,I1,M6,E5,P17);(A3,I1,M6,E5,P18);(A3,I1,M6,E5,P19);(A3,I1,M6,E5,P20);(A3,I1,M6,E6,P1);(A3,I1,M6,E6,P2);(A3,I1,M6,E6,P3);(A3,I1,M6,E6,P4);(A3,I1,M6,E6,P5);(A3,I1,M6,E6,P6);(A3,I1,M6,E6,P7);(A3,I1,M6,E6,P8);(A3,I1,M6,E6,P9);(A3,I1,M6,E6,P10);(A3,I1,M6,E6,P11);(A3,I1,M6,E6,P12);(A3,I1,M6,E6,P13);(A3,I1,M6,E6,P14);(A3,I1,M6,E6,P15);(A3,I1,M6,E6,P16);(A3,I1,M6,E6,P17);(A3,I1,M6,E6,P18);(A3,I1,M6,E6,P19);(A3,I1,M6,E6,P20);(A3,I1,M6,E7,P1);(A3,I1,M6,E7,P2);(A3,I1,M6,E7,P3);(A3,I1,M6,E7,P4);(A3,I1,M6,E7,P5);(A3,I1,M6,E7,P6);(A3,I1,M6,E7,P7);(A3,I1,M6,E7,P8);(A3,I1,M6,E7,P9);(A3,I1,M6,E7,P10);(A3,I1,M6,E7,P11);(A3,I1,M6,E7,P12);(A3,I1,M6,E7,P13);(A3,I1,M6,E7,P14);(A3,I1,M6,E7,P15);(A3,I1,M6,E7,P16);(A3,I1,M6,E7,P17);(A3,I1,M6,E7,P18);(A3,I1,M6,E7,P19);(A3,I1,M6,E7,P20);(A3,I1,M6,E8,P1);(A3,I1,M6,E8,P2);(A3,I1,M6,E8,P3);(A3,I1,M6,E8,P4);(A3,I1,M6,E8,P5);(A3,I1,M6,E8,P6);(A3,I1,M6,E8,P7);(A3,I1,M6,E8,P8);(A3,I1,M6,E8,P9);(A3,I1,M6,E8,P10);(A3,I1,M6,E8,P11);(A3,I1,M6,E8,P12);(A3,I1,M6,E8,P13);(A3,I1,M6,E8,P14);(A3,I1,M6,E8,P15);(A3,I1,M6,E8,P16);(A3,I1,M6,E8,P17);(A3,I1,M6,E8,P18);(A3,I1,M6,E8,P19);(A3,I1,M6,E8,P20);(A3,I1,M6,E9,P1);(A3,I1,M6,E9,P2);(A3,I1,M6,E9,P3);(A3,I1,M6,E9,P4);(A3,I1,M6,E9,P5);(A3,I1,M6,E9,P6);(A3,I1,M6,E9,P7);(A3,I1,M6,E9,P8);(A3,I1,M6,E9,P9);(A3,I1,M6,E9,P10);(A3,I1,M6,E9,P11);(A3,I1,M6,E9,P12);(A3,I1,M6,E9,P13);(A3,I1,M6,E9,P14);(A3,I1,M6,E9,P15);(A3,I1,M6,E9,P16);(A3,I1,M6,E9,P17);(A3,I1,M6,E9,P18);(A3,I1,M6,E9,P19);(A3,I1,M6,E9,P20);(A3,I2,M1,E1,P1);(A3,I2,M1,E1,P2);(A3,I2,M1,E1,P3);(A3,I2,M1,E1,P4);(A3,I2,M1,E1,P5);(A3,I2,M1,E1,P6);(A3,I2,M1,E1,P7);(A3,I2,M1,E1,P8);(A3,I2,M1,E1,P9);(A3,I2,M1,E1,P10);(A3,I2,M1,E1,P11);(A3,I2,M1,E1,P12);(A3,I2,M1,E1,P13);(A3,I2,M1,E1,P14);(A3,I2,M1,E1,P15);(A3,I2,M1,E1,P16);(A3,I2,M1,E1,P17);(A3,I2,M1,E1,P18);(A3,I2,M1,E1,P19);(A3,I2,M1,E1,P20);(A3,I2,M1,E2,P1);(A3,I2,M1,E2,P2);(A3,I2,M1,E2,P3);(A3,I2,M1,E2,P4);(A3,I2,M1,E2,P5);(A3,I2,M1,E2,P6);(A3,I2,M1,E2,P7);(A3,I2,M1,E2,P8);(A3,I2,M1,E2,P9);(A3,I2,M1,E2,P10);(A3,I2,M1,E2,P11);(A3,I2,M1,E2,P12);(A3,I2,M1,E2,P13);(A3,I2,M1,E2,P14);(A3,I2,M1,E2,P15);(A3,I2,M1,E2,P16);(A3,I2,M1,E2,P17);(A3,I2,M1,E2,P18);(A3,I2,M1,E2,P19);(A3,I2,M1,E2,P20);(A3,I2,M1,E3,P1);(A3,I2,M1,E3,P2);(A3,I2,M1,E3,P3);(A3,I2,M1,E3,P4);(A3,I2,M1,E3,P5);(A3,I2,M1,E3,P6);(A3,I2,M1,E3,P7);(A3,I2,M1,E3,P8);(A3,I2,M1,E3,P9);(A3,I2,M1,E3,P10);(A3,I2,M1,E3,P11);(A3,I2,M1,E3,P12);(A3,I2,M1,E3,P13);(A3,I2,M1,E3,P14);(A3,I2,M1,E3,P15);(A3,I2,M1,E3,P16);(A3,I2,M1,E3,P17);(A3,I2,M1,E3,P18);(A3,I2,M1,E3,P19);(A3,I2,M1,E3,P20);(A3,I2,M1,E4,P1);(A3,I2,M1,E4,P2);(A3,I2,M1,E4,P3);(A3,I2,M1,E4,P4);(A3,I2,M1,E4,P5);(A3,I2,M1,E4,P6);(A3,I2,M1,E4,P7);(A3,I2,M1,E4,P8);(A3,I2,M1,E4,P9);(A3,I2,M1,E4,P10);(A3,I2,M1,E4,P11);(A3,I2,M1,E4,P12);(A3,I2,M1,E4,P13);(A3,I2,M1,E4,P14);(A3,I2,M1,E4,P15);(A3,I2,M1,E4,P16);(A3,I2,M1,E4,P17);(A3,I2,M1,E4,P18);(A3,I2,M1,E4,P19);(A3,I2,M1,E4,P20);(A3,I2,M1,E5,P1);(A3,I2,M1,E5,P2);(A3,I2,M1,E5,P3);(A3,I2,M1,E5,P4);(A3,I2,M1,E5,P5);(A3,I2,M1,E5,P6);(A3,I2,M1,E5,P7);(A3,I2,M1,E5,P8);(A3,I2,M1,E5,P9);(A3,I2,M1,E5,P10);(A3,I2,M1,E5,P11);(A3,I2,M1,E5,P12);(A3,I2,M1,E5,P13);(A3,I2,M1,E5,P14);(A3,I2,M1,E5,P15);(A3,I2,M1,E5,P16);(A3,I2,M1,E5,P17);(A3,I2,M1,E5,P18);(A3,I2,M1,E5,P19);(A3,I2,M1,E5,P20);(A3,I2,M1,E6,P1);(A3,I2,M1,E6,P2);(A3,I2,M1,E6,P3);(A3,I2,M1,E6,P4);(A3,I2,M1,E6,P5);(A3,I2,M1,E6,P6);(A3,I2,M1,E6,P7);(A3,I2,M1,E6,P8);(A3,I2,M1,E6,P9);(A3,I2,M1,E6,P10);(A3,I2,M1,E6,P11);(A3,I2,M1,E6,P12);(A3,I2,M1,E6,P13);(A3,I2,M1,E6,P14);(A3,I2,M1,E6,P15);(A3,I2,M1,E6,P16);(A3,I2,M1,E6,P17);(A3,I2,M1,E6,P18);(A3,I2,M1,E6,P19);(A3,I2,M1,E6,P20);(A3,I2,M1,E7,P1);(A3,I2,M1,E7,P2);(A3,I2,M1,E7,P3);(A3,I2,M1,E7,P4);(A3,I2,M1,E7,P5);(A3,I2,M1,E7,P6);(A3,I2,M1,E7,P7);(A3,I2,M1,E7,P8);(A3,I2,M1,E7,P9);(A3,I2,M1,E7,P10);(A3,I2,M1,E7,P11);(A3,I2,M1,E7,P12);(A3,I2,M1,E7,P13);(A3,I2,M1,E7,P14);(A3,I2,M1,E7,P15);(A3,I2,M1,E7,P16);(A3,I2,M1,E7,P17);(A3,I2,M1,E7,P18);(A3,I2,M1,E7,P19);(A3,I2,M1,E7,P20);(A3,I2,M1,E8,P1);(A3,I2,M1,E8,P2);(A3,I2,M1,E8,P3);(A3,I2,M1,E8,P4);(A3,I2,M1,E8,P5);(A3,I2,M1,E8,P6);(A3,I2,M1,E8,P7);(A3,I2,M1,E8,P8);(A3,I2,M1,E8,P9);(A3,I2,M1,E8,P10);(A3,I2,M1,E8,P11);(A3,I2,M1,E8,P12);(A3,I2,M1,E8,P13);(A3,I2,M1,E8,P14);(A3,I2,M1,E8,P15);(A3,I2,M1,E8,P16);(A3,I2,M1,E8,P17);(A3,I2,M1,E8,P18);(A3,I2,M1,E8,P19);(A3,I2,M1,E8,P20);(A3,I2,M1,E9,P1);(A3,I2,M1,E9,P2);(A3,I2,M1,E9,P3);(A3,I2,M1,E9,P4);(A3,I2,M1,E9,P5);(A3,I2,M1,E9,P6);(A3,I2,M1,E9,P7);(A3,I2,M1,E9,P8);(A3,I2,M1,E9,P9);(A3,I2,M1,E9,P10);(A3,I2,M1,E9,P11);(A3,I2,M1,E9,P12);(A3,I2,M1,E9,P13);(A3,I2,M1,E9,P14);(A3,I2,M1,E9,P15);(A3,I2,M1,E9,P16);(A3,I2,M1,E9,P17);(A3,I2,M1,E9,P18);(A3,I2,M1,E9,P19);(A3,I2,M1,E9,P20);(A3,I2,M2,E1,P1);(A3,I2,M2,E1,P2);(A3,I2,M2,E1,P3);(A3,I2,M2,E1,P4);(A3,I2,M2,E1,P5);(A3,I2,M2,E1,P6);(A3,I2,M2,E1,P7);(A3,I2,M2,E1,P8);(A3,I2,M2,E1,P9);(A3,I2,M2,E1,P10);(A3,I2,M2,E1,P11);(A3,I2,M2,E1,P12);(A3,I2,M2,E1,P13);(A3,I2,M2,E1,P14);(A3,I2,M2,E1,P15);(A3,I2,M2,E1,P16);(A3,I2,M2,E1,P17);(A3,I2,M2,E1,P18);(A3,I2,M2,E1,P19);(A3,I2,M2,E1,P20);(A3,I2,M2,E2,P1);(A3,I2,M2,E2,P2);(A3,I2,M2,E2,P3);(A3,I2,M2,E2,P4);(A3,I2,M2,E2,P5);(A3,I2,M2,E2,P6);(A3,I2,M2,E2,P7);(A3,I2,M2,E2,P8);(A3,I2,M2,E2,P9);(A3,I2,M2,E2,P10);(A3,I2,M2,E2,P11);(A3,I2,M2,E2,P12);(A3,I2,M2,E2,P13);(A3,I2,M2,E2,P14);(A3,I2,M2,E2,P15);(A3,I2,M2,E2,P16);(A3,I2,M2,E2,P17);(A3,I2,M2,E2,P18);(A3,I2,M2,E2,P19);(A3,I2,M2,E2,P20);(A3,I2,M2,E3,P1);(A3,I2,M2,E3,P2);(A3,I2,M2,E3,P3);(A3,I2,M2,E3,P4);(A3,I2,M2,E3,P5);(A3,I2,M2,E3,P6);(A3,I2,M2,E3,P7);(A3,I2,M2,E3,P8);(A3,I2,M2,E3,P9);(A3,I2,M2,E3,P10);(A3,I2,M2,E3,P11);(A3,I2,M2,E3,P12);(A3,I2,M2,E3,P13);(A3,I2,M2,E3,P14);(A3,I2,M2,E3,P15);(A3,I2,M2,E3,P16);(A3,I2,M2,E3,P17);(A3,I2,M2,E3,P18);(A3,I2,M2,E3,P19);(A3,I2,M2,E3,P20);(A3,I2,M2,E4,P1);(A3,I2,M2,E4,P2);(A3,I2,M2,E4,P3);(A3,I2,M2,E4,P4);(A3,I2,M2,E4,P5);(A3,I2,M2,E4,P6);(A3,I2,M2,E4,P7);(A3,I2,M2,E4,P8);(A3,I2,M2,E4,P9);(A3,I2,M2,E4,P10);(A3,I2,M2,E4,P11);(A3,I2,M2,E4,P12);(A3,I2,M2,E4,P13);(A3,I2,M2,E4,P14);(A3,I2,M2,E4,P15);(A3,I2,M2,E4,P16);(A3,I2,M2,E4,P17);(A3,I2,M2,E4,P18);(A3,I2,M2,E4,P19);(A3,I2,M2,E4,P20);(A3,I2,M2,E5,P1);(A3,I2,M2,E5,P2);(A3,I2,M2,E5,P3);(A3,I2,M2,E5,P4);(A3,I2,M2,E5,P5);(A3,I2,M2,E5,P6);(A3,I2,M2,E5,P7);(A3,I2,M2,E5,P8);(A3,I2,M2,E5,P9);(A3,I2,M2,E5,P10);(A3,I2,M2,E5,P11);(A3,I2,M2,E5,P12);(A3,I2,M2,E5,P13);(A3,I2,M2,E5,P14);(A3,I2,M2,E5,P15);(A3,I2,M2,E5,P16);(A3,I2,M2,E5,P17);(A3,I2,M2,E5,P18);(A3,I2,M2,E5,P19);(A3,I2,M2,E5,P20);(A3,I2,M2,E6,P1);(A3,I2,M2,E6,P2);(A3,I2,M2,E6,P3);(A3,I2,M2,E6,P4);(A3,I2,M2,E6,P5);(A3,I2,M2,E6,P6);(A3,I2,M2,E6,P7);(A3,I2,M2,E6,P8);(A3,I2,M2,E6,P9);(A3,I2,M2,E6,P10);(A3,I2,M2,E6,P11);(A3,I2,M2,E6,P12);(A3,I2,M2,E6,P13);(A3,I2,M2,E6,P14);(A3,I2,M2,E6,P15);(A3,I2,M2,E6,P16);(A3,I2,M2,E6,P17);(A3,I2,M2,E6,P18);(A3,I2,M2,E6,P19);(A3,I2,M2,E6,P20);(A3,I2,M2,E7,P1);(A3,I2,M2,E7,P2);(A3,I2,M2,E7,P3);(A3,I2,M2,E7,P4);(A3,I2,M2,E7,P5);(A3,I2,M2,E7,P6);(A3,I2,M2,E7,P7);(A3,I2,M2,E7,P8);(A3,I2,M2,E7,P9);(A3,I2,M2,E7,P10);(A3,I2,M2,E7,P11);(A3,I2,M2,E7,P12);(A3,I2,M2,E7,P13);(A3,I2,M2,E7,P14);(A3,I2,M2,E7,P15);(A3,I2,M2,E7,P16);(A3,I2,M2,E7,P17);(A3,I2,M2,E7,P18);(A3,I2,M2,E7,P19);(A3,I2,M2,E7,P20);(A3,I2,M2,E8,P1);(A3,I2,M2,E8,P2);(A3,I2,M2,E8,P3);(A3,I2,M2,E8,P4);(A3,I2,M2,E8,P5);(A3,I2,M2,E8,P6);(A3,I2,M2,E8,P7);(A3,I2,M2,E8,P8);(A3,I2,M2,E8,P9);(A3,I2,M2,E8,P10);(A3,I2,M2,E8,P11);(A3,I2,M2,E8,P12);(A3,I2,M2,E8,P13);(A3,I2,M2,E8,P14);(A3,I2,M2,E8,P15);(A3,I2,M2,E8,P16);(A3,I2,M2,E8,P17);(A3,I2,M2,E8,P18);(A3,I2,M2,E8,P19);(A3,I2,M2,E8,P20);(A3,I2,M2,E9,P1);(A3,I2,M2,E9,P2);(A3,I2,M2,E9,P3);(A3,I2,M2,E9,P4);(A3,I2,M2,E9,P5);(A3,I2,M2,E9,P6);(A3,I2,M2,E9,P7);(A3,I2,M2,E9,P8);(A3,I2,M2,E9,P9);(A3,I2,M2,E9,P10);(A3,I2,M2,E9,P11);(A3,I2,M2,E9,P12);(A3,I2,M2,E9,P13);(A3,I2,M2,E9,P14);(A3,I2,M2,E9,P15);(A3,I2,M2,E9,P16);(A3,I2,M2,E9,P17);(A3,I2,M2,E9,P18);(A3,I2,M2,E9,P19);(A3,I2,M2,E9,P20);(A3,I2,M3,E1,P1);(A3,I2,M3,E1,P2);(A3,I2,M3,E1,P3);(A3,I2,M3,E1,P4);(A3,I2,M3,E1,P5);(A3,I2,M3,E1,P6);(A3,I2,M3,E1,P7);(A3,I2,M3,E1,P8);(A3,I2,M3,E1,P9);(A3,I2,M3,E1,P10);(A3,I2,M3,E1,P11);(A3,I2,M3,E1,P12);(A3,I2,M3,E1,P13);(A3,I2,M3,E1,P14);(A3,I2,M3,E1,P15);(A3,I2,M3,E1,P16);(A3,I2,M3,E1,P17);(A3,I2,M3,E1,P18);(A3,I2,M3,E1,P19);(A3,I2,M3,E1,P20);(A3,I2,M3,E2,P1);(A3,I2,M3,E2,P2);(A3,I2,M3,E2,P3);(A3,I2,M3,E2,P4);(A3,I2,M3,E2,P5);(A3,I2,M3,E2,P6);(A3,I2,M3,E2,P7);(A3,I2,M3,E2,P8);(A3,I2,M3,E2,P9);(A3,I2,M3,E2,P10);(A3,I2,M3,E2,P11);(A3,I2,M3,E2,P12);(A3,I2,M3,E2,P13);(A3,I2,M3,E2,P14);(A3,I2,M3,E2,P15);(A3,I2,M3,E2,P16);(A3,I2,M3,E2,P17);(A3,I2,M3,E2,P18);(A3,I2,M3,E2,P19);(A3,I2,M3,E2,P20);(A3,I2,M3,E3,P1);(A3,I2,M3,E3,P2);(A3,I2,M3,E3,P3);(A3,I2,M3,E3,P4);(A3,I2,M3,E3,P5);(A3,I2,M3,E3,P6);(A3,I2,M3,E3,P7);(A3,I2,M3,E3,P8);(A3,I2,M3,E3,P9);(A3,I2,M3,E3,P10);(A3,I2,M3,E3,P11);(A3,I2,M3,E3,P12);(A3,I2,M3,E3,P13);(A3,I2,M3,E3,P14);(A3,I2,M3,E3,P15);(A3,I2,M3,E3,P16);(A3,I2,M3,E3,P17);(A3,I2,M3,E3,P18);(A3,I2,M3,E3,P19);(A3,I2,M3,E3,P20);(A3,I2,M3,E4,P1);(A3,I2,M3,E4,P2);(A3,I2,M3,E4,P3);(A3,I2,M3,E4,P4);(A3,I2,M3,E4,P5);(A3,I2,M3,E4,P6);(A3,I2,M3,E4,P7);(A3,I2,M3,E4,P8);(A3,I2,M3,E4,P9);(A3,I2,M3,E4,P10);(A3,I2,M3,E4,P11);(A3,I2,M3,E4,P12);(A3,I2,M3,E4,P13);(A3,I2,M3,E4,P14);(A3,I2,M3,E4,P15);(A3,I2,M3,E4,P16);(A3,I2,M3,E4,P17);(A3,I2,M3,E4,P18);(A3,I2,M3,E4,P19);(A3,I2,M3,E4,P20);(A3,I2,M3,E5,P1);(A3,I2,M3,E5,P2);(A3,I2,M3,E5,P3);(A3,I2,M3,E5,P4);(A3,I2,M3,E5,P5);(A3,I2,M3,E5,P6);(A3,I2,M3,E5,P7);(A3,I2,M3,E5,P8);(A3,I2,M3,E5,P9);(A3,I2,M3,E5,P10);(A3,I2,M3,E5,P11);(A3,I2,M3,E5,P12);(A3,I2,M3,E5,P13);(A3,I2,M3,E5,P14);(A3,I2,M3,E5,P15);(A3,I2,M3,E5,P16);(A3,I2,M3,E5,P17);(A3,I2,M3,E5,P18);(A3,I2,M3,E5,P19);(A3,I2,M3,E5,P20);(A3,I2,M3,E6,P1);(A3,I2,M3,E6,P2);(A3,I2,M3,E6,P3);(A3,I2,M3,E6,P4);(A3,I2,M3,E6,P5);(A3,I2,M3,E6,P6);(A3,I2,M3,E6,P7);(A3,I2,M3,E6,P8);(A3,I2,M3,E6,P9);(A3,I2,M3,E6,P10);(A3,I2,M3,E6,P11);(A3,I2,M3,E6,P12);(A3,I2,M3,E6,P13);(A3,I2,M3,E6,P14);(A3,I2,M3,E6,P15);(A3,I2,M3,E6,P16);(A3,I2,M3,E6,P17);(A3,I2,M3,E6,P18);(A3,I2,M3,E6,P19);(A3,I2,M3,E6,P20);(A3,I2,M3,E7,P1);(A3,I2,M3,E7,P2);(A3,I2,M3,E7,P3);(A3,I2,M3,E7,P4);(A3,I2,M3,E7,P5);(A3,I2,M3,E7,P6);(A3,I2,M3,E7,P7);(A3,I2,M3,E7,P8);(A3,I2,M3,E7,P9);(A3,I2,M3,E7,P10);(A3,I2,M3,E7,P11);(A3,I2,M3,E7,P12);(A3,I2,M3,E7,P13);(A3,I2,M3,E7,P14);(A3,I2,M3,E7,P15);(A3,I2,M3,E7,P16);(A3,I2,M3,E7,P17);(A3,I2,M3,E7,P18);(A3,I2,M3,E7,P19);(A3,I2,M3,E7,P20);(A3,I2,M3,E8,P1);(A3,I2,M3,E8,P2);(A3,I2,M3,E8,P3);(A3,I2,M3,E8,P4);(A3,I2,M3,E8,P5);(A3,I2,M3,E8,P6);(A3,I2,M3,E8,P7);(A3,I2,M3,E8,P8);(A3,I2,M3,E8,P9);(A3,I2,M3,E8,P10);(A3,I2,M3,E8,P11);(A3,I2,M3,E8,P12);(A3,I2,M3,E8,P13);(A3,I2,M3,E8,P14);(A3,I2,M3,E8,P15);(A3,I2,M3,E8,P16);(A3,I2,M3,E8,P17);(A3,I2,M3,E8,P18);(A3,I2,M3,E8,P19);(A3,I2,M3,E8,P20);(A3,I2,M3,E9,P1);(A3,I2,M3,E9,P2);(A3,I2,M3,E9,P3);(A3,I2,M3,E9,P4);(A3,I2,M3,E9,P5);(A3,I2,M3,E9,P6);(A3,I2,M3,E9,P7);(A3,I2,M3,E9,P8);(A3,I2,M3,E9,P9);(A3,I2,M3,E9,P10);(A3,I2,M3,E9,P11);(A3,I2,M3,E9,P12);(A3,I2,M3,E9,P13);(A3,I2,M3,E9,P14);(A3,I2,M3,E9,P15);(A3,I2,M3,E9,P16);(A3,I2,M3,E9,P17);(A3,I2,M3,E9,P18);(A3,I2,M3,E9,P19);(A3,I2,M3,E9,P20);(A3,I2,M4,E1,P1);(A3,I2,M4,E1,P2);(A3,I2,M4,E1,P3);(A3,I2,M4,E1,P4);(A3,I2,M4,E1,P5);(A3,I2,M4,E1,P6);(A3,I2,M4,E1,P7);(A3,I2,M4,E1,P8);(A3,I2,M4,E1,P9);(A3,I2,M4,E1,P10);(A3,I2,M4,E1,P11);(A3,I2,M4,E1,P12);(A3,I2,M4,E1,P13);(A3,I2,M4,E1,P14);(A3,I2,M4,E1,P15);(A3,I2,M4,E1,P16);(A3,I2,M4,E1,P17);(A3,I2,M4,E1,P18);(A3,I2,M4,E1,P19);(A3,I2,M4,E1,P20);(A3,I2,M4,E2,P1);(A3,I2,M4,E2,P2);(A3,I2,M4,E2,P3);(A3,I2,M4,E2,P4);(A3,I2,M4,E2,P5);(A3,I2,M4,E2,P6);(A3,I2,M4,E2,P7);(A3,I2,M4,E2,P8);(A3,I2,M4,E2,P9);(A3,I2,M4,E2,P10);(A3,I2,M4,E2,P11);(A3,I2,M4,E2,P12);(A3,I2,M4,E2,P13);(A3,I2,M4,E2,P14);(A3,I2,M4,E2,P15);(A3,I2,M4,E2,P16);(A3,I2,M4,E2,P17);(A3,I2,M4,E2,P18);(A3,I2,M4,E2,P19);(A3,I2,M4,E2,P20);(A3,I2,M4,E3,P1);(A3,I2,M4,E3,P2);(A3,I2,M4,E3,P3);(A3,I2,M4,E3,P4);(A3,I2,M4,E3,P5);(A3,I2,M4,E3,P6);(A3,I2,M4,E3,P7);(A3,I2,M4,E3,P8);(A3,I2,M4,E3,P9);(A3,I2,M4,E3,P10);(A3,I2,M4,E3,P11);(A3,I2,M4,E3,P12);(A3,I2,M4,E3,P13);(A3,I2,M4,E3,P14);(A3,I2,M4,E3,P15);(A3,I2,M4,E3,P16);(A3,I2,M4,E3,P17);(A3,I2,M4,E3,P18);(A3,I2,M4,E3,P19);(A3,I2,M4,E3,P20);(A3,I2,M4,E4,P1);(A3,I2,M4,E4,P2);(A3,I2,M4,E4,P3);(A3,I2,M4,E4,P4);(A3,I2,M4,E4,P5);(A3,I2,M4,E4,P6);(A3,I2,M4,E4,P7);(A3,I2,M4,E4,P8);(A3,I2,M4,E4,P9);(A3,I2,M4,E4,P10);(A3,I2,M4,E4,P11);(A3,I2,M4,E4,P12);(A3,I2,M4,E4,P13);(A3,I2,M4,E4,P14);(A3,I2,M4,E4,P15);(A3,I2,M4,E4,P16);(A3,I2,M4,E4,P17);(A3,I2,M4,E4,P18);(A3,I2,M4,E4,P19);(A3,I2,M4,E4,P20);(A3,I2,M4,E5,P1);(A3,I2,M4,E5,P2);(A3,I2,M4,E5,P3);(A3,I2,M4,E5,P4);(A3,I2,M4,E5,P5);(A3,I2,M4,E5,P6);(A3,I2,M4,E5,P7);(A3,I2,M4,E5,P8);(A3,I2,M4,E5,P9);(A3,I2,M4,E5,P10);(A3,I2,M4,E5,P11);(A3,I2,M4,E5,P12);(A3,I2,M4,E5,P13);(A3,I2,M4,E5,P14);(A3,I2,M4,E5,P15);(A3,I2,M4,E5,P16);(A3,I2,M4,E5,P17);(A3,I2,M4,E5,P18);(A3,I2,M4,E5,P19);(A3,I2,M4,E5,P20);(A3,I2,M4,E6,P1);(A3,I2,M4,E6,P2);(A3,I2,M4,E6,P3);(A3,I2,M4,E6,P4);(A3,I2,M4,E6,P5);(A3,I2,M4,E6,P6);(A3,I2,M4,E6,P7);(A3,I2,M4,E6P8);(A3,I2,M4,E6,P9);(A3,I2,M4,E6,P10);(A3,I2,M4,E6,P11);(A3,I2,M4,E6,P12);(A3,I2,M4,E6,P13);(A3,I2,M4,E6,P14);(A3,I2,M4,E6,P15);(A3,I2,M4,E6,P16);(A3,I2,M4,E6,P17);(A3,I2,M4,E6,P18);(A3,I2,M4,E6,P19);(A3,I2,M4,E6,P20);(A3,I2,M4,E7,P1);(A3,I2,M4,E7,P2);(A3,I2,M4,E7,P3);(A3,I2,M4,E7,P4);(A3,I2,M4,E7,P5);(A3,I2,M4,E7,P6);(A3,I2,M4,E7,P7);(A3,I2,M4,E7,P8);(A3,I2,M4,E7,P9);(A3,I2,M4,E7,P10);(A3,I2,M4,E7,P11);(A3,I2,M4,E7,P12);(A3,I2,M4,E7,P13);(A3,I2,M4,E7,P14);(A3,I2,M4,E7,P15);(A3,I2,M4,E7,P16);(A3,I2,M4,E7,P17);(A3,I2,M4,E7,P18);(A3,I2,M4,E7,P19);(A3,I2,M4,E7,P20);(A3,I2,M4,E8,P1);(A3,I2,M4,E8,P2);(A3,I2,M4,E8,P3);(A3,I2,M4,E8,P4);(A3,I2,M4,E8,P5);(A3,I2,M4,E8,P6);(A3,I2,M4,E8,P7);(A3,I2,M4,E8,P8);(A3,I2,M4,E8,P9);(A3,I2,M4,E8,P10);(A3,I2,M4,E8,P11);(A3,I2,M4,E8,P12);(A3,I2,M4,E8,P13);(A3,I2,M4,E8,P14);(A3,I2,M4,E8,P15);(A3,I2,M4,E8,P16);(A3,I2,M4,E8,P17);(A3,I2,M4,E8,P18);(A3,I2,M4,E8,P19);(A3,I2,M4,E8,P20);(A3,I2,M4,E9,P1);(A3,I2,M4,E9,P2);(A3,I2,M4,E9,P3);(A3,I2,M4,E9,P4);(A3,I2,M4,E9,P5);(A3,I2,M4,E9,P6);(A3,I2,M4,E9,P7);(A3,I2,M4,E9,P8);(A3,I2,M4,E9,P9);(A3,I2,M4,E9,P10);(A3,I2,M4,E9,P11);(A3,I2,M4,E9,P12);(A3,I2,M4,E9,P13);(A3,I2,M4,E9,P14);(A3,I2,M4,E9,P15);(A3,I2,M4,E9,P16);(A3,I2,M4,E9,P17);(A3,I2,M4,E9,P18);(A3,I2,M4,E9,P19);(A3,I2,M4,E9,P20);(A3,I2,M5,E1,P1);(A3,I2,M5,E1,P2);(A3,I2,M5,E1,P3);(A3,I2,M5,E1,P4);(A3,I2,M5,E1,P5);(A3,I2,M5,E1,P6);(A3,I2,M5,E1,P7);(A3,I2,M5,E1,P8);(A3,I2,M5,E1,P9);(A3,I2,M5,E1,P10);(A3,I2,M5,E1,P11);(A3,I2,M5,E1,P12);(A3,I2,M5,E1,P13);(A3,I2,M5,E1,P14);(A3,I2,M5,E1,P15);(A3,I2,M5,E1,P16);(A3,I2,M5,E1,P17);(A3,I2,M5,E1,P18);(A3,I2,M5,E1,P19);(A3,I2,M5,E1,P20);(A3,I2,M5,E2,P1);(A3,I2,M5,E2,P2);(A3,I2,M5,E2,P3);(A3,I2,M5,E2,P4);(A3,I2,M5,E2,P5);(A3,I2,M5,E2,P6);(A3,I2,M5,E2,P7);(A3,I2,M5,E2,P8);(A3,I2,M5,E2,P9);(A3,I2,M5,E2,P10);(A3,I2,M5,E2,P11);(A3,I2,M5,E2,P12);(A3,I2,M5,E2,P13);(A3,I2,M5,E2,P14);(A3,I2,M5,E2,P15);(A3,I2,M5,E2,P16);(A3,I2,M5,E2,P17);(A3,I2,M5,E2,P18);(A3,I2,M5,E2,P19);(A3,I2,M5,E2,P20);(A3,I2,M5,E3,P1);(A3,I2,M5,E3,P2);(A3,I2,M5,E3,P3);(A3,I2,M5,E3,P4);(A3,I2,M5,E3,P5);(A3,I2,M5,E3,P6);(A3,I2,M5,E3,P7);(A3,I2,M5,E3,P8);(A3,I2,M5,E3,P9);(A3,I2,M5,E3,P10);(A3,I2,M5,E3,P11);(A3,I2,M5,E3,P12);(A3,I2,M5,E3,P13);(A3,I2,M5,E3,P14);(A3,I2,M5,E3,P15);(A3,I2,M5,E3,P16);(A3,I2,M5,E3,P17);(A3,I2,M5,E3,P18);(A3,I2,M5,E3,P19);(A3,I2,M5,E3,P20);(A3,I2,M5,E4,P1);(A3,I2,M5,E4,P2);(A3,I2,M5,E4,P3);(A3,I2,M5,E4,P4);(A3,I2,M5,E4,P5);(A3,I2,M5,E4,P6);(A3,I2,M5,E4,P7);(A3,I2,M5,E4,P8);(A3,I2,M5,E4,P9);(A3,I2,M5,E4,P10);(A3,I2,M5,E4,P11);(A3,I2,M5,E4,P12);(A3,I2,M5,E4,P13);(A3,I2,M5,E4,P14);(A3,I2,M5,E4,P15);(A3,I2,M5,E4,P16);(A3,I2,M5,E4,P17);(A3,I2,M5,E4,P18);(A3,I2,M5,E4,P19);(A3,I2,M5,E4,P20);(A3,I2,M5,E5,P1);(A3,I2,M5,E5,P2);(A3,I2,M5,E5,P3);(A3,I2,M5,E5,P4);(A3,I2,M5,E5,P5);(A3,I2,M5,E5,P6);(A3,I2,M5,E5,P7);(A3,I2,M5,E5,P8);(A3,I2,M5,E5,P9);(A3,I2,M5,E5,P10);(A3,I2,M5,E5,P11);(A3,I2,M5,E5,P12);(A3,I2,M5,E5,P13);(A3,I2,M5,E5,P14);(A3,I2,M5,E5,P15);(A3,I2,M5,E5,P16);(A3,I2,M5,E5,P17);(A3,I2,M5,E5,P18);(A3,I2,M5,E5,P19);(A3,I2,M5,E5,P20);(A3,I2,M5,E6,P1);(A3,I2,M5,E6,P2);(A3,I2,M5,E6,P3);(A3,I2,M5,E6,P4);(A3,I2,M5,E6,P5);(A3,I2,M5,E6,P6);(A3,I2,M5,E6,P7);(A3,I2,M5,E6,P8);(A3,I2,M5,E6,P9);(A3,I2,M5,E6,P10);(A3,I2,M5,E6,P11);(A3,I2,M5,E6,P12);(A3,I2,M5,E6,P13);(A3,I2,M5,E6,P14);(A3,I2,M5,E6,P15);(A3,I2,M5,E6,P16);(A3,I2,M5,E6,P17);(A3,I2,M5,E6,P18);(A3,I2,M5,E6,P19);(A3,I2,M5,E6,P20);(A3,I2,M5,E7,P1);(A3,I2,M5,E7,P2);(A3,I2,M5,E7,P3);(A3,I2,M5,E7,P4);(A3,I2,M5,E7,P5);(A3,I2,M5,E7,P6);(A3,I2,M5,E7,P7);(A3,I2,M5,E7,P8);(A3,I2,M5,E7,P9);(A3,I2,M5,E7,P10);(A3,I2,M5,E7,P11);(A3,I2,M5,E7,P12);(A3,I2,M5,E7,P13);(A3,I2,M5,E7,P14);(A3,I2,M5,E7,P15);(A3,I2,M5,E7,P16);(A3,I2,M5,E7,P17);(A3,I2,M5,E7,P18);(A3,I2,M5,E7,P19);(A3,I2,M5,E7,P20);(A3,I2,M5,E8,P1);(A3,I2,M5,E8,P2);(A3,I2,M5,E8,P3);(A3,I2,M5,E8,P4);(A3,I2,M5,E8,P5);(A3,I2,M5,E8,P6);(A3,I2,M5,E8,P7);(A3,I2,M5,E8,P8);(A3,I2,M5,E8,P9);(A3,I2,M5,E8,P10);(A3,I2,M5,E8,P11);(A3,I2,M5,E8,P12);(A3,I2,M5,E8,P13);(A3,I2,M5,E8,P14);(A3,I2,M5,E8,P15);(A3,I2,M5,E8,P16);(A3,I2,M5,E8,P17);(A3,I2,M5,E8,P18);(A3,I2,M5,E8,P19);(A3,I2,M5,E8,P20);(A3,I2,M5,E9,P1);(A3,I2,M5,E9,P2);(A3,I2,M5,E9,P3);(A3,I2,M5,E9,P4);(A3,I2,M5,E9,P5);(A3,I2,M5,E9,P6);(A3,I2,M5,E9,P7);(A3,I2,M5,E9,P8);(A3,I2,M5,E9,P9);(A3,I2,M5,E9,P10);(A3,I2,M5,E9,P11);(A3,I2,M5,E9,P12);(A3,I2,M5,E9,P13);(A3,I2,M5,E9,P14);(A3,I2,M5,E9,P15);(A3,I2,M5,E9,P16);(A3,I2,M5,E9,P17);(A3,I2,M5,E9,P18);(A3,I2,M5,E9,P19);(A3,I2,M5,E9,P20);(A3,I2,M6,E1,P1);(A3,I2,M6,E1,P2);(A3,I2,M6,E1,P3);(A3,I2,M6,E1,P4);(A3,I2,M6,E1,P5);(A3,I2,M6,E1,P6);(A3,I2,M6,E1,P7);(A3,I2,M6,E1,P8);(A3,I2,M6,E1,P9);(A3,I2,M6,E1,P10);(A3,I2,M6,E1,P11);(A3,I2,M6,E1,P12);(A3,I2,M6,E1,P13);(A3,I2,M6,E1,P14);(A3,I2,M6,E1,P15);(A3,I2,M6,E1,P16);(A3,I2,M6,E1,P17);(A3,I2,M6,E1,P18);(A3,I2,M6,E1,P19);(A3,I2,M6,E1,P20);(A3,I2,M6,E2,P1);(A3,I2,M6,E2,P2);(A3,I2,M6,E2,P3);(A3,I2,M6,E2,P4);(A3,I2,M6,E2,P5);(A3,I2,M6,E2,P6);(A3,I2,M6,E2,P7);(A3,I2,M6,E2,P8);(A3,I2,M6,E2,P9);(A3,I2,M6,E2,P10);(A3,I2,M6,E2,P11);(A3,I2,M6,E2,P12);(A3,I2,M6,E2,P13);(A3,I2,M6,E2,P14);(A3,I2,M6,E2,P15);(A3,I2,M6,E2,P16);(A3,I2,M6,E2,P17);(A3,I2,M6,E2,P18);(A3,I2,M6,E2,P19);(A3,I2,M6,E2,P20);(A3,I2,M6,E3,P1);(A3,I2,M6,E3,P2);(A3,I2,M6,E3,P3);(A3,I2,M6,E3,P4);(A3,I2,M6,E3,P5);(A3,I2,M6,E3,P6);(A3,I2,M6,E3,P7);(A3,I2,M6,E3,P8);(A3,I2,M6,E3,P9);(A3,I2,M6,E3,P10);(A3,I2,M6,E3,P11);(A3,I2,M6,E3,P12);(A3,I2,M6,E3,P13);(A3,I2,M6,E3,P14);(A3,I2,M6,E3,P15);(A3,I2,M6,E3,P16);(A3,I2,M6,E3,P17);(A3,I2,M6,E3,P18);(A3,I2,M6,E3,P19);(A3,I2,M6,E3,P20);(A3,I2,M6,E4,P1);(A3,I2,M6,E4,P2);(A3,I2,M6,E4,P3);(A3,I2,M6,E4,P4);(A3,I2,M6,E4,P5);(A3,I2,M6,E4,P6);(A3,I2,M6,E4,P7);(A3,I2,M6,E4,P8);(A3,I2,M6,E4,P9);(A3,I2,M6,E4,P10);(A3,I2,M6,E4,P11);(A3,I2,M6,E4,P12);(A3,I2,M6,E4,P13);(A3,I2,M6,E4,P14);(A3,I2,M6,E4,P15);(A3,I2,M6,E4,P16);(A3,I2,M6,E4,P17);(A3,I2,M6,E4,P18);(A3,I2,M6,E4,P19);(A3,I2,M6,E4,P20);(A3,I2,M6,E5,P1);(A3,I2,M6,E5,P2);(A3,I2,M6,E5,P3);(A3,I2,M6,E5,P4);(A3,I2,M6,E5,P5);(A3,I2,M6,E5,P6);(A3,I2,M6,E5,P7);(A3,I2,M6,E5,P8);(A3,I2,M6,E5,P9);(A3,I2,M6,E5,P10);(A3,I2,M6,E5,P11);(A3,I2,M6,E5,P12);(A3,I2,M6,E5,P13);(A3,I2,M6,E5,P14);(A3,I2,M6,E5,P15);(A3,I2,M6,E5,P16);(A3,I2,M6,E5,P17);(A3,I2,M6,E5,P18);(A3,I2,M6,E5,P19);(A3,I2,M6,E5,P20);(A3,I2,M6,E6,P1);(A3,I2,M6,E6,P2);(A3,I2,M6,E6,P3);(A3,I2,M6,E6,P4);(A3,I2,M6,E6,P5);(A3,I2,M6,E6,P6);(A3,I2,M6,E6,P7);(A3,I2,M6,E6,P8);(A3,I2,M6,E6,P9);(A3,I2,M6,E6,P10);(A3,I2,M6,E6,P11);(A3,I2,M6,E6,P12);(A3,I2,M6,E6,P13);(A3,I2,M6,E6,P14);(A3,I2,M6,E6,P15);(A3,I2,M6,E6,P16);(A3,I2,M6,E6,P17);(A3,I2,M6,E6,P18);(A3,I2,M6,E6,P19);(A3,I2,M6,E6,P20);(A3,I2,M6,E7,P1);(A3,I2,M6,E7,P2);(A3,I2,M6,E7,P3);(A3,I2,M6,E7,P4);(A3,I2,M6,E7,P5);(A3,I2,M6,E7,P6);(A3,I2,M6,E7,P7);(A3,I2,M6,E7,P8);(A3,I2,M6,E7,P9);(A3,I2,M6,E7,P10);(A3,I2,M6,E7,P11);(A3,I2,M6,E7,P12);(A3,I2,M6,E7,P13);(A3,I2,M6,E7,P14);(A3,I2,M6,E7,P15);(A3,I2,M6,E7,P16);(A3,I2,M6,E7,P17);(A3,I2,M6,E7,P18);(A3,I2,M6,E7,P19);(A3,I2,M6,E7,P20);(A3,I2,M6,E8,P1);(A3,I2,M6,E8,P2);(A3,I2,M6,E8,P3);(A3,I2,M6,E8,P4);(A3,I2,M6,E8,P5);(A3,I2,M6,E8,P6);(A3,I2,M6,E8,P7);(A3,I2,M6,E8,P8);(A3,I2,M6,E8,P9);(A3,I2,M6,E8,P10);(A3,I2,M6,E8,P11);(A3,I2,M6,E8,P12);(A3,I2,M6,E8,P13);(A3,I2,M6,E8,P14);(A3,I2,M6,E8,P15);(A3,I2,M6,E8,P16);(A3,I2,M6,E8,P17);(A3,I2,M6,E8,P18);(A3,I2,M6,E8,P19);(A3,I2,M6,E8,P20);(A3,I2,M6,E9,P1);(A3,I2,M6,E9,P2);(A3,I2,M6,E9,P3);(A3,I2,M6,E9,P4);(A3,I2,M6,E9,P5);(A3,I2,M6,E9,P6);(A3,I2,M6,E9,P7);(A3,I2,M6,E9,P8);(A3,I2,M6,E9,P9);(A3,I2,M6,E9,P10);(A3,I2,M6,E9,P11);(A3,I2,M6,E9,P12);(A3,I2,M6,E9,P13);(A3,I2,M6,E9,P14);(A3,I2,M6,E9,P15);(A3,I2,M6,E9,P16);(A3,I2,M6,E9,P17);(A3,I2,M6,E9,P18);(A3,I2,M6,E9,P19);(A3,I2,M6,E9,P20);(A3,I3,M1,E1,P1);(A3,I3,M1,E1,P2);(A3,I3,M1,E1,P3);(A3,I3,M1,E1,P4);(A3,I3,M1,E1,P5);(A3,I3,M1,E1,P6);(A3,I3,M1,E1,P7);(A3,I3,M1,E1,P8);(A3,I3,M1,E1,P9);(A3,I3,M1,E1,P10);(A3,I3,M1,E1,P11);(A3,I3,M1,E1,P12);(A3,I3,M1,E1,P13);(A3,I3,M1,E1,P14);(A3,I3,M1,E1,P15);(A3,I3,M1,E1,P16);(A3,I3,M1,E1,P17);(A3,I3,M1,E1,P18);(A3,I3,M1,E1,P19);(A3,I3,M1,E1,P20);(A3,I3,M1,E2,P1);(A3,I3,M1,E2,P2);(A3,I3,M1,E2,P3);(A3,I3,M1,E2,P4);(A3,I3,M1,E2,P5);(A3,I3,M1,E2,P6);(A3,I3,M1,E2,P7);(A3,I3,M1,E2,P8);(A3,I3,M1,E2,P9);(A3,I3,M1,E2,P10);(A3,I3,M1,E2,P11);(A3,I3,M1,E2,P12);(A3,I3,M1,E2,P13);(A3,I3,M1,E2,P14);(A3,I3,M1,E2,P15);(A3,I3,M1,E2,P16);(A3,I3,M1,E2,P17);(A3,I3,M1,E2,P18);(A3,I3,M1,E2,P19);(A3,I3,M1,E2,P20);(A3,I3,M1,E3,P1);(A3,I3,M1,E3,P2);(A3,I3,M1,E3,P3);(A3,I3,M1,E3,P4);(A3,I3,M1,E3,P5);(A3,I3,M1,E3,P6);(A3,I3,M1,E3,P7);(A3,I3,M1,E3,P8);(A3,I3,M1,E3,P9);(A3,I3,M1,E3,P10);(A3,I3,M1,E3,P11);(A3,I3,M1,E3,P12);(A3,I3,M1,E3,P13);(A3,I3,M1,E3,P14);(A3,I3,M1,E3,P15);(A3,I3,M1,E3,P16);(A3,I3,M1,E3,P17);(A3,I3,M1,E3,P18);(A3,I3,M1,E3,P19);(A3,I3,M1,E3,P20);(A3,I3,M1,E4,P1);(A3,I3,M1,E4,P2);(A3,I3,M1,E4,P3);(A3,I3,M1,E4,P4);(A3,I3,M1,E4,P5);(A3,I3,M1,E4,P6);(A3,I3,M1,E4,P7);(A3,I3,M1,E4,P8);(A3,I3,M1,E4,P9);(A3,I3,M1,E4,P10);(A3,I3,M1,E4,P11);(A3,I3,M1,E4,P12);(A3,I3,M1,E4,P13);(A3,I3,M1,E4,P14);(A3,I3,M1,E4,P15);(A3,I3,M1,E4,P16);(A3,I3,M1,E4,P17);(A3,I3,M1,E4,P18);(A3,I3,M1,E4,P19);(A3,I3,M1,E4,P20);(A3,I3,M1,E5,P1);(A3,I3,M1,E5,P2);(A3,I3,M1,E5,P3);(A3,I3,M1,E5,P4);(A3,I3,M1,E5,P5);(A3,I3,M1,E5,P6);(A3,I3,M1,E5,P7);(A3,I3,M1,E5,P8);(A3,I3,M1,E5,P9);(A3,I3,M1,E5,P10);(A3,I3,M1,E5,P11);(A3,I3,M1,E5,P12);(A3,I3,M1,E5,P13);(A3,I3,M1,E5,P14);(A3,I3,M1,E5,P15);(A3,I3,M1,E5,P16);(A3,I3,M1,E5,P17);(A3,I3,M1,E5,P18);(A3,I3,M1,E5,P19);(A3,I3,M1,E5,P20);(A3,I3,M1,E6,P1);(A3,I3,M1,E6,P2);(A3,I3,M1,E6,P3);(A3,I3,M1,E6,P4);(A3,I3,M1,E6,P5);(A3,I3,M1,E6,P6);(A3,I3,M1,E6,P7);(A3,I3,M1,E6,P8);(A3,I3,M1,E6,P9);(A3,I3,M1,E6,P10);(A3,I3,M1,E6,P11);(A3,I3,M1,E6,P12);(A3,I3,M1,E6,P13);(A3,I3,M1,E6,P14);(A3,I3,M1,E6,P15);(A3,I3,M1,E6,P16);(A3,I3,M1,E6,P17);(A3,I3,M1,E6,P18);(A3,I3,M1,E6,P19);(A3,I3,M1,E6,P20);(A3,I3,M1,E7,P1);(A3,I3,M1,E7,P2);(A3,I3,M1,E7,P3);(A3,I3,M1,E7,P4);(A3,I3,M1,E7,P5);(A3,I3,M1,E7,P6);(A3,I3,M1,E7,P7);(A3,I3,M1,E7,P8);(A3,I3,M1,E7,P9);(A3,I3,M1,E7,P10);(A3,I3,M1,E7,P11);(A3,I3,M1,E7,P12);(A3,I3,M1,E7,P13);(A3,I3,M1,E7,P14);(A3,I3,M1,E7,P15);(A3,I3,M1,E7,P16);(A3,I3,M1,E7,P17);(A3,I3,M1,E7,P18);(A3,I3,M1,E7,P19);(A3,I3,M1,E7,P20);(A3,I3,M1,E8,P1);(A3,I3,M1,E8,P2);(A3,I3,M1,E8,P3);(A3,I3,M1,E8,P4);(A3,I3,M1,E8,P5);(A3,I3,M1,E8,P6);(A3,I3,M1,E8,P7);(A3,I3,M1,E8,P8);(A3,I3,M1,E8,P9);(A3,I3,M1,E8,P10);(A3,I3,M1,E8,P11);(A3,I3,M1,E8,P12);(A3,I3,M1,E8,P13);(A3,I3,M1,E8,P14);(A3,I3,M1,E8,P15);(A3,I3,M1,E8,P16);(A3,I3,M1,E8,P17);(A3,I3,M1,E8,P18);(A3,I3,M1,E8,P19);(A3,I3,M1,E8,P20);(A3,I3,M1,E9,P1);(A3,I3,M1,E9,P2);(A3,I3,M1,E9,P3);(A3,I3,M1,E9,P4);(A3,I3,M1,E9,P5);(A3,I3,M1,E9,P6);(A3,I3,M1,E9,P7);(A3,I3,M1,E9,P8);(A3,I3,M1,E9,P9);(A3,I3,M1,E9,P10);(A3,I3,M1,E9,P11);(A3,I3,M1,E9,P12);(A3,I3,M1,E9,P13);(A3,I3,M1,E9,P14);(A3,I3,M1,E9,P15);(A3,I3,M1,E9,P16);(A3,I3,M1,E9,P17);(A3,I3,M1,E9,P18);(A3,I3,M1,E9,P19);(A3,I3,M1,E9,P20);(A3,I3,M2,E1,P1);(A3,I3,M2,E1,P2);(A3,I3,M2,E1,P3);(A3,I3,M2,E1,P4);(A3,I3,M2,E1,P5);(A3,I3,M2,E1,P6);(A3,I3,M2,E1,P7);(A3,I3,M2,E1,P8);(A3,I3,M2,E1,P9);(A3,I3,M2,E1,P10);(A3,I3,M2,E1,P11);(A3,I3,M2,E1,P12);(A3,I3,M2,E1,P13);(A3,I3,M2,E1,P14);(A3,I3,M2,E1,P15);(A3,I3,M2,E1,P16);(A3,I3,M2,E1,P17);(A3,I3,M2,E1,P18);(A3,I3,M2,E1,P19);(A3,I3,M2,E1,P20);(A3,I3,M2,E2,P1);(A3,I3,M2,E2,P2);(A3,I3,M2,E2,P3);(A3,I3,M2,E2,P4);(A3,I3,M2,E2,P5);(A3,I3,M2,E2,P6);(A3,I3,M2,E2,P7);(A3,I3,M2,E2,P8);(A3,I3,M2,E2,P9);(A3,I3,M2,E2,P10);(A3,I3,M2,E2,P11);(A3,I3,M2,E2,P12);(A3,I3,M2,E2,P13);(A3,I3,M2,E2,P14);(A3,I3,M2,E2,P15);(A3,I3,M2,E2,P16);(A3,I3,M2,E2,P17);(A3,I3,M2,E2,P18);(A3,I3,M2,E2,P19);(A3,I3,M2,E2,P20);(A3,I3,M2,E3,P1);(A3,I3,M2,E3,P2);(A3,I3,M2,E3,P3);(A3,I3,M2,E3,P4);(A3,I3,M2,E3,P5);(A3,I3,M2,E3,P6);(A3,I3,M2,E3,P7);(A3,I3,M2,E3,P8);(A3,I3,M2,E3,P9);(A3,I3,M2,E3,P10);(A3,I3,M2,E3,P11);(A3,I3,M2,E3,P12);(A3,I3,M2,E3,P13);(A3,I3,M2,E3,P14);(A3,I3,M2,E3,P15);(A3,I3,M2,E3,P16);(A3,I3,M2,E3,P17);(A3,I3,M2,E3,P18);(A3,I3,M2,E3,P19);(A3,I3,M2,E3,P20);(A3,I3,M2,E4,P1);(A3,I3,M2,E4,P2);(A3,I3,M2,E4,P3);(A3,I3,M2,E4,P4);(A3,I3,M2,E4,P5);(A3,I3,M2,E4,P6);(A3,I3,M2,E4,P7);(A3,I3,M2,E4,P8);(A3,I3,M2,E4,P9);(A3,I3,M2,E4,P10);(A3,I3,M2,E4,P11);(A3,I3,M2,E4,P12);(A3,I3,M2,E4,P13);(A3,I3,M2,E4,P14);(A3,I3,M2,E4,P15);(A3,I3,M2,E4,P16);(A3,I3,M2,E4,P17);(A3,I3,M2,E4,P18);(A3,I3,M2,E4,P19);(A3,I3,M2,E4,P20);(A3,I3,M2,E5,P1);(A3,I3,M2,E5,P2);(A3,I3,M2,E5,P3);(A3,I3,M2,E5,P4);(A3,I3,M2,E5,P5);(A3,I3,M2,E5,P6);(A3,I3,M2,E5,P7);(A3,I3,M2,E5,P8);(A3,I3,M2,E5,P9);(A3,I3,M2,E5,P10);(A3,I3,M2,E5,P11);(A3,I3,M2,E5,P12);(A3,I3,M2,E5,P13);(A3,I3,M2,E5,P14);(A3,I3,M2,E5,P15);(A3,I3,M2,E5,P16);(A3,I3,M2,E5,P17);(A3,I3,M2,E5,P18);(A3,I3,M2,E5,P19);(A3,I3,M2,E5,P20);(A3,I3,M2,E6,P1);(A3,I3,M2,E6,P2);(A3,I3,M2,E6,P3);(A3,I3,M2,E6,P4);(A3,I3,M2,E6,P5);(A3,I3,M2,E6,P6);(A3,I3,M2,E6,P7);(A3,I3,M2,E6,P8);(A3,I3,M2,E6,P9);(A3,I3,M2,E6,P10);(A3,I3,M2,E6,P11);(A3,I3,M2,E6,P12);(A3,I3,M2,E6,P13);(A3,I3,M2,E6,P14);(A3,I3,M2,E6,P15);(A3,I3,M2,E6,P16);(A3,I3,M2,E6,P17);(A3,I3,M2,E6,P18);(A3,I3,M2,E6,P19);(A3,I3,M2,E6,P20);(A3,I3,M2,E7,P1);(A3,I3,M2,E7,P2);(A3,I3,M2,E7,P3);(A3,I3,M2,E7,P4);(A3,I3,M2,E7,P5);(A3,I3,M2,E7,P6);(A3,I3,M2,E7,P7);(A3,I3,M2,E7,P8);(A3,I3,M2,E7,P9);(A3,I3,M2,E7,P10);(A3,I3,M2,E7,P11);(A3,I3,M2,E7,P12);(A3,I3,M2,E7,P13);(A3,I3,M2,E7,P14);(A3,I3,M2,E7,P15);(A3,I3,M2,E7,P16);(A3,I3,M2,E7,P17);(A3,I3,M2,E7,P18);(A3,I3,M2,E7,P19);(A3,I3,M2,E7,P20);(A3,I3,M2,E8,P1);(A3,I3,M2,E8,P2);(A3,I3,M2,E8,P3);(A3,I3,M2,E8,P4);(A3,I3,M2,E8,P5);(A3,I3,M2,E8,P6);(A3,I3,M2,E8,P7);(A3,I3,M2,E8,P8);(A3,I3,M2,E8,P9);(A3,I3,M2,E8,P10);(A3,I3,M2,E8,P11);(A3,I3,M2,E8,P12);(A3,I3,M2,E8,P13);(A3,I3,M2,E8,P14);(A3,I3,M2,E8,P15);(A3,I3,M2,E8,P16);(A3,I3,M2,E8,P17);(A3,I3,M2,E8,P18);(A3,I3,M2,E8,P19);(A3,I3,M2,E8,P20);(A3,I3,M2,E9,P1);(A3,I3,M2,E9,P2);(A3,I3,M2,E9,P3);(A3,I3,M2,E9,P4);(A3,I3,M2,E9,P5);(A3,I3,M2,E9,P6);(A3,I3,M2,E9,P7);(A3,I3,M2,E9,P8);(A3,I3,M2,E9,P9);(A3,I3,M2,E9,P10);(A3,I3,M2,E9,P11);(A3,I3,M2,E9,P12);(A3,I3,M2,E9,P13);(A3,I3,M2,E9,P14);(A3,I3,M2,E9,P15);(A3,I3,M2,E9,P16);(A3,I3,M2,E9,P17);(A3,I3,M2,E9,P18);(A3,I3,M2,E9,P19);(A3,I3,M2,E9,P20);(A3,I3,M3,E1,P1);(A3,I3,M3,E1,P2);(A3,I3,M3,E1,P3);(A3,I3,M3,E1,P4);(A3,I3,M3,E1,P5);(A3,I3,M3,E1,P6);(A3,I3,M3,E1,P7);(A3,I3,M3,E1,P8);(A3,I3,M3,E1,P9);(A3,I3,M3,E1,P10);(A3,I3,M3,E1,P11);(A3,I3,M3,E1,P12);(A3,I3,M3,E1,P13);(A3,I3,M3,E1,P14);(A3,I3,M3,E1,P15);(A3,I3,M3,E1,P16);(A3,I3,M3,E1,P17);(A3,I3,M3,E1,P18);(A3,I3,M3,E1,P19);(A3,I3,M3,E1,P20);(A3,I3,M3,E2,P1);(A3,I3,M3,E2,P2);(A3,I3,M3,E2,P3);(A3,I3,M3,E2,P4);(A3,I3,M3,E2,P5);(A3,I3,M3,E2,P6);(A3,I3,M3,E2,P7);(A3,I3,M3,E2,P8);(A3,I3,M3,E2,P9);(A3,I3,M3,E2,P10);(A3,I3,M3,E2,P11);(A3,I3,M3,E2,P12);(A3,I3,M3,E2,P13);(A3,I3,M3,E2,P14);(A3,I3,M3,E2,P15);(A3,I3,M3,E2,P16);(A3,I3,M3,E2,P17);(A3,I3,M3,E2,P18);(A3,I3,M3,E2,P19);(A3,I3,M3,E2,P20);(A3,I3,M3,E3,P1);(A3,I3,M3,E3,P2);(A3,I3,M3,E3,P3);(A3,I3,M3,E3,P4);(A3,I3,M3,E3,P5);(A3,I3,M3,E3,P6);(A3,I3,M3,E3,P7);(A3,I3,M3,E3,P8);(A3,I3,M3,E3,P9);(A3,I3,M3,E3,P10);(A3,I3,M3,E3,P11);(A3,I3,M3,E3,P12);(A3,I3,M3,E3,P13);(A3,I3,M3,E3,P14);(A3,I3,M3,E3,P15);(A3,I3,M3,E3,P16);(A3,I3,M3,E3,P17);(A3,I3,M3,E3,P18);(A3,I3,M3,E3,P19);(A3,I3,M3,E3,P20);(A3,I3,M3,E4,P1);(A3,I3,M3,E4,P2);(A3,I3,M3,E4,P3);(A3,I3,M3,E4,P4);(A3,I3,M3,E4,P5);(A3,I3,M3,E4,P6);(A3,I3,M3,E4,P7);(A3,I3,M3,E4,P8);(A3,I3,M3,E4,P9);(A3,I3,M3,E4,P10);(A3,I3,M3,E4,P11);(A3,I3,M3,E4,P12);(A3,I3,M3,E4,P13);(A3,I3,M3,E4,P14);(A3,I3,M3,E4,P15);(A3,I3,M3,E4,P16);(A3,I3,M3,E4,P17);(A3,I3,M3,E4,P18);(A3,I3,M3,E4,P19);(A3,I3,M3,E4,P20);(A3,I3,M3,E5,P1);(A3,I3,M3,E5,P2);(A3,I3,M3,E5,P3);(A3,I3,M3,E5,P4);(A3,I3,M3,E5,P5);(A3,I3,M3,E5,P6);(A3,I3,M3,E5,P7);(A3,I3,M3,E5,P8);(A3,I3,M3,E5,P9);(A3,I3,M3,E5,P10);(A3,I3,M3,E5,P11);(A3,I3,M3,E5,P12);(A3,I3,M3,E5,P13);(A3,I3,M3,E5,P14);(A3,I3,M3,E5,P15);(A3,I3,M3,E5,P16);(A3,I3,M3,E5,P17);(A3,I3,M3,E5,P18);(A3,I3,M3,E5,P19);(A3,I3,M3,E5,P20);(A3,I3,M3,E6,P1);(A3,I3,M3,E6,P2);(A3,I3,M3,E6,P3);(A3,I3,M3,E6,P4);(A3,I3,M3,E6,P5);(A3,I3,M3,E6,P6);(A3,I3,M3,E6,P7);(A3,I3,M3,E6,P8);(A3,I3,M3,E6,P9);(A3,I3,M3,E6,P10);(A3,I3,M3,E6,P11);(A3,I3,M3,E6,P12);(A3,I3,M3,E6,P13);(A3,I3,M3,E6,P14);(A3,I3,M3,E6,P15);(A3,I3,M3,E6,P16);(A3,I3,M3,E6,P17);(A3,I3,M3,E6,P18);(A3,I3,M3,E6,P19);(A3,I3,M3,E6,P20);(A3,I3,M3,E7,P1);(A3,I3,M3,E7,P2);(A3,I3,M3,E7,P3);(A3,I3,M3,E7,P4);(A3,I3,M3,E7,P5);(A3,I3,M3,E7,P6);(A3,I3,M3,E7,P7);(A3,I3,M3,E7,P8);(A3,I3,M3,E7,P9);(A3,I3,M3,E7,P10);(A3,I3,M3,E7,P11);(A3,I3,M3,E7,P12);(A3,I3,M3,E7,P13);(A3,I3,M3,E7,P14);(A3,I3,M3,E7,P15);(A3,I3,M3,E7,P16);(A3,I3,M3,E7,P17);(A3,I3,M3,E7,P18);(A3,I3,M3,E7,P19);(A3,I3,M3,E7,P20);(A3,I3,M3,E8,P1);(A3,I3,M3,E8,P2);(A3,I3,M3,E8,P3);(A3,I3,M3,E8,P4);(A3,I3,M3,E8,P5);(A3,I3,M3,E8,P6);(A3,I3,M3,E8,P7);(A3,I3,M3,E8,P8);(A3,I3,M3,E8,P9);(A3,I3,M3,E8,P10);(A3,I3,M3,E8,P11);(A3,I3,M3,E8,P12);(A3,I3,M3,E8,P13);(A3,I3,M3,E8,P14);(A3,I3,M3,E8,P15);(A3,I3,M3,E8,P16);(A3,I3,M3,E8,P17);(A3,I3,M3,E8,P18);(A3,I3,M3,E8,P19);(A3,I3,M3,E8,P20);(A3,I3,M3,E9,P1);(A3,I3,M3,E9,P2);(A3,I3,M3,E9,P3);(A3,I3,M3,E9,P4);(A3,I3,M3,E9,P5);(A3,I3,M3,E9,P6);(A3,I3,M3,E9,P7);(A3,I3,M3,E9,P8);(A3,I3,M3,E9,P9);(A3,I3,M3,E9,P10);(A3,I3,M3,E9,P11);(A3,I3,M3,E9,P12);(A3,I3,M3,E9,P13);(A3,I3,M3,E9,P14);(A3,I3,M3,E9,P15);(A3,I3,M3,E9,P16);(A3,I3,M3,E9,P17);(A3,I3,M3,E9,P18);(A3,I3,M3,E9,P19);(A3,I3,M3,E9,P20);(A3,I3,M4,E1,P1);(A3,I3,M4,E1,P2);(A3,I3,M4,E1,P3);(A3,I3,M4,E1,P4);(A3,I3,M4,E1,P5);(A3,I3,M4,E1,P6);(A3,I3,M4,E1,P7);(A3,I3,M4,E1,P8);(A3,I3,M4,E1,P9);(A3,I3,M4,E1,P10);(A3,I3,M4,E1,P11);(A3,I3,M4,E1,P12);(A3,I3,M4,E1,P13);(A3,I3,M4,E1,P14);(A3,I3,M4,E1,P15);(A3,I3,M4,E1,P16);(A3,I3,M4,E1,P17);(A3,I3,M4,E1,P18);(A3,I3,M4,E1,P19);(A3,I3,M4,E1,P20);(A3,I3,M4,E2,P1);(A3,I3,M4,E2,P2);(A3,I3,M4,E2,P3);(A3,I3,M4,E2,P4);(A3,I3,M4,E2,P5);(A3,I3,M4,E2,P6);(A3,I3,M4,E2,P7);(A3,I3,M4,E2,P8);(A3,I3,M4,E2,P9);(A3,I3,M4,E2,P10);(A3,I3,M4,E2,P11);(A3,I3,M4,E2,P12);(A3,I3,M4,E2,P13);(A3,I3,M4,E2,P14);(A3,I3,M4,E2,P15);(A3,I3,M4,E2,P16);(A3,I3,M4,E2,P17);(A3,I3,M4,E2,P18);(A3,I3,M4,E2,P19);(A3,I3,M4,E2,P20);(A3,I3,M4,E3,P1);(A3,I3,M4,E3,P2);(A3,I3,M4,E3,P3);(A3,I3,M4,E3,P4);(A3,I3,M4,E3,P5);(A3,I3,M4,E3,P6);(A3,I3,M4,E3,P7);(A3,I3,M4,E3,P8);(A3,I3,M4,E3,P9);(A3,I3,M4,E3,P10);(A3,I3,M4,E3,P11);(A3,I3,M4,E3,P12);(A3,I3,M4,E3,P13);(A3,I3,M4,E3,P14);(A3,I3,M4,E3,P15);(A3,I3,M4,E3,P16);(A3,I3,M4,E3,P17);(A3,I3,M4,E3,P18);(A3,I3,M4,E3,P19);(A3,I3,M4,E3,P20);(A3,I3,M4,E4,P1);(A3,I3,M4,E4,P2);(A3,I3,M4,E4,P3);(A3,I3,M4,E4,P4);(A3,I3,M4,E4,P5);(A3,I3,M4,E4,P6);(A3,I3,M4,E4,P7);(A3,I3,M4,E4,P8);(A3,I3,M4,E4,P9);(A3,I3,M4,E4,P10);(A3,I3,M4,E4,P11);(A3,I3,M4,E4,P12);(A3,I3,M4,E4,P13);(A3,I3,M4,E4,P14);(A3,I3,M4,E4,P15);(A3,I3,M4,E4,P16);(A3,I3,M4,E4,P17);(A3,I3,M4,E4,P18);(A3,I3,M4,E4,P19);(A3,I3,M4,E4,P20);(A3,I3,M4,E5,P1);(A3,I3,M4,E5,P2);(A3,I3,M4,E5,P3);(A3,I3,M4,E5,P4);(A3,I3,M4,E5,P5);(A3,I3,M4,E5,P6);(A3,I3,M4,E5,P7);(A3,I3,M4,E5,P8);(A3,I3,M4,E5,P9);(A3,I3,M4,E5,P10);(A3,I3,M4,E5,P11);(A3,I3,M4,E5,P12);(A3,I3,M4,E5,P13);(A3,I3,M4,E5,P14);(A3,I3,M4,E5,P15);(A3,I3,M4,E5,P16);(A3,I3,M4,E5,P17);(A3,I3,M4,E5,P18);(A3,I3,M4,E5,P19);(A3,I3,M4,E5,P20);(A3,I3,M4,E6,P1);(A3,I3,M4,E6,P2);(A3,I3,M4,E6,P3);(A3,I3,M4,E6,P4);(A3,I3,M4,E6,P5);(A3,I3,M4,E6,P6);(A3,I3,M4,E6,P7);(A3,I3,M4,E6,P8);(A3,I3,M4,E6,P9);(A3,I3,M4,E6,P10);(A3,I3,M4,E6,P11);(A3,I3,M4,E6,P12);(A3,I3,M4,E6,P13);(A3,I3,M4,E6,P14);(A3,I3,M4,E6,P15);(A3,I3,M4,E6,P16);(A3,I3,M4,E6,P17);(A3,I3,M4,E6,P18);(A3,I3,M4,E6,P19);(A3,I3,M4,E6,P20);(A3,I3,M4,E7,P1);(A3,I3,M4,E7,P2);(A3,I3,M4,E7,P3);(A3,I3,M4,E7,P4);(A3,I3,M4,E7,P5);(A3,I3,M4,E7,P6);(A3,I3,M4,E7,P7);(A3,I3,M4,E7,P8);(A3,I3,M4,E7,P9);(A3,I3,M4,E7,P10);(A3,I3,M4,E7,P11);(A3,I3,M4,E7,P12);(A3,I3,M4,E7,P13);(A3,I3,M4,E7,P14);(A3,I3,M4,E7,P15);(A3,I3,M4,E7,P16);(A3,I3,M4,E7,P17);(A3,I3,M4,E7,P18);(A3,I3,M4,E7,P19);(A3,I3,M4,E7,P20);(A3,I3,M4,E8,P1);(A3,I3,M4,E8,P2);(A3,I3,M4,E8,P3);(A3,I3,M4,E8,P4);(A3,I3,M4,E8,P5);(A3,I3,M4,E8,P6);(A3,I3,M4,E8,P7);(A3,I3,M4,E8,P8);(A3,I3,M4,E8,P9);(A3,I3,M4,E8,P10);(A3,I3,M4,E8,P11);(A3,I3,M4,E8,P12);(A3,I3,M4,E8,P13);(A3,I3,M4,E8,P14);(A3,I3,M4,E8,P15);(A3,I3,M4,E8,P16);(A3,I3,M4,E8,P17);(A3,I3,M4,E8,P18);(A3,I3,M4,E8,P19);(A3,I3,M4,E8,P20);(A3,I3,M4,E9,P1);(A3,I3,M4,E9,P2);(A3,I3,M4,E9,P3);(A3,I3,M4,E9,P4);(A3,I3,M4,E9,P5);(A3,I3,M4,E9,P6);(A3,I3,M4,E9,P7);(A3,I3,M4,E9,P8);(A3,I3,M4,E9,P9);(A3,I3,M4,E9,P10);(A3,I3,M4,E9,P11);(A3,I3,M4,E9,P12);(A3,I3,M4,E9,P13);(A3,I3,M4,E9,P14);(A3,I3,M4,E9,P15);(A3,I3,M4,E9,P16);(A3,I3,M4,E9,P17);(A3,I3,M4,E9,P18);(A3,I3,M4,E9,P19);(A3,I3,M4,E9,P20);(A3,I3,M5,E1,P1);(A3,I3,M5,E1,P2);(A3,I3,M5,E1,P3);(A3,I3,M5,E1,P4);(A3,I3,M5,E1,P5);(A3,I3,M5,E1,P6);(A3,I3,M5,E1,P7);(A3,I3,M5,E1,P8);(A3,I3,M5,E1,P9);(A3,I3,M5,E1,P10);(A3,I3,M5,E1,P11);(A3,I3,M5,E1,P12);(A3,I3,M5,E1,P13);(A3,I3,M5,E1,P14);(A3,I3,M5,E1,P15);(A3,I3,M5,E1,P16);(A3,I3,M5,E1,P17);(A3,I3,M5,E1,P18);(A3,I3,M5,E1,P19);(A3,I3,M5,E1,P20);(A3,I3,M5,E2,P1);(A3,I3,M5,E2,P2);(A3,I3,M5,E2,P3);(A3,I3,M5,E2,P4);(A3,I3,M5,E2,P5);(A3,I3,M5,E2,P6);(A3,I3,M5,E2,P7);(A3,I3,M5,E2,P8);(A3,I3,M5,E2,P9);(A3,I3,M5,E2,P10);(A3,I3,M5,E2,P11);(A3,I3,M5,E2,P12);(A3,I3,M5,E2,P13);(A3,I3,M5,E2,P14);(A3,I3,M5,E2,P15);(A3,I3,M5,E2,P16);(A3,I3,M5,E2,P17);(A3,I3,M5,E2,P18);(A3,I3,M5,E2,P19);(A3,I3,M5,E2,P20);(A3,I3,M5,E3,P1);(A3,I3,M5,E3,P2);(A3,I3,M5,E3,P3);(A3,I3,M5,E3,P4);(A3,I3,M5,E3,P5);(A3,I3,M5,E3,P6);(A3,I3,M5,E3,P7);(A3,I3,M5,E3,P8);(A3,I3,M5,E3,P9);(A3,I3,M5,E3,P10);(A3,I3,M5,E3,P11);(A3,I3,M5,E3,P12);(A3,I3,M5,E3,P13);(A3,I3,M5,E3,P14);(A3,I3,M5,E3,P15);(A3,I3,M5,E3,P16);(A3,I3,M5,E3,P17);(A3,I3,M5,E3,P18);(A3,I3,M5,E3,P19);(A3,I3,M5,E3,P20);(A3,I3,M5,E4,P1);(A3,I3,M5,E4,P2);(A3,I3,M5,E4,P3);(A3,I3,M5,E4,P4);(A3,I3,M5,E4,P5);(A3,I3,M5,E4,P6);(A3,I3,M5,E4,P7);(A3,I3,M5,E4,P8);(A3,I3,M5,E4,P9);(A3,I3,M5,E4,P10);(A3,I3,M5,E4,P11);(A3,I3,M5,E4,P12);(A3,I3,M5,E4,P13);(A3,I3,M5,E4,P14);(A3,I3,M5,E4,P15);(A3,I3,M5,E4,P16);(A3,I3,M5,E4,P17);(A3,I3,M5,E4,P18);(A3,I3,M5,E4,P19);(A3,I3,M5,E4,P20);(A3,I3,M5,E5,P1);(A3,I3,M5,E5,P2);(A3,I3,M5,E5,P3);(A3,I3,M5,E5,P4);(A3,I3,M5,E5,P5);(A3,I3,M5,E5,P6);(A3,I3,M5,E5,P7);(A3,I3,M5,E5,P8);(A3,I3,M5,E5,P9);(A3,I3,M5,E5,P10);(A3,I3,M5,E5,P11);(A3,I3,M5,E5,P12);(A3,I3,M5,E5,P13);(A3,I3,M5,E5,P14);(A3,I3,M5,E5,P15);(A3,I3,M5,E5,P16);(A3,I3,M5,E5,P17);(A3,I3,M5,E5,P18);(A3,I3,M5,E5,P19);(A3,I3,M5,E5,P20);(A3,I3,M5,E6,P1);(A3,I3,M5,E6,P2);(A3,I3,M5,E6,P3);(A3,I3,M5,E6,P4);(A3,I3,M5,E6,P5);(A3,I3,M5,E6,P6);(A3,I3,M5,E6,P7);(A3,I3,M5,E6,P8);(A3,I3,M5,E6,P9);(A3,I3,M5,E6,P10);(A3,I3,M5,E6,P11);(A3,I3,M5,E6,P12);(A3,I3,M5,E6,P13);(A3,I3,M5,E6,P14);(A3,I3,M5,E6,P15);(A3,I3,M5,E6,P16);(A3,I3,M5,E6,P17);(A3,I3,M5,E6,P18);(A3,I3,M5,E6,P19);(A3,I3,M5,E6,P20);(A3,I3,M5,E7,P1);(A3,I3,M5,E7,P2);(A3,I3,M5,E7,P3);(A3,I3,M5,E7,P4);(A3,I3,M5,E7,P5);(A3,I3,M5,E7,P6);(A3,I3,M5,E7,P7);(A3,I3,M5,E7,P8);(A3,I3,M5,E7,P9);(A3,I3,M5,E7,P10);(A3,I3,M5,E7,P11);(A3,I3,M5,E7,P12);(A3,I3,M5,E7,P13);(A3,I3,M5,E7,P14);(A3,I3,M5,E7,P15);(A3,I3,M5,E7,P16);(A3,I3,M5,E7,P17);(A3,I3,M5,E7,P18);(A3,I3,M5,E7,P19);(A3,I3,M5,E7,P20);(A3,I3,M5,E8,P1);(A3,I3,M5,E8,P2);(A3,I3,M5,E8,P3);(A3,I3,M5,E8,P4);(A3,I3,M5,E8,P5);(A3,I3,M5,E8,P6);(A3,I3,M5,E8,P7);(A3,I3,M5,E8,P8);(A3,I3,M5,E8,P9);(A3,I3,M5,E8,P10);(A3,I3,M5,E8,P11);(A3,I3,M5,E8,P12);(A3,I3,M5,E8,P13);(A3,I3,M5,E8,P14);(A3,I3,M5,E8,P15);(A3,I3,M5,E8,P16);(A3,I3,M5,E8,P17);(A3,I3,M5,E8,P18);(A3,I3,M5,E8,P19);(A3,I3,M5,E8,P20);(A3,I3,M5,E9,P1);(A3,I3,M5,E9,P2);(A3,I3,M5,E9,P3);(A3,I3,M5,E9,P4);(A3,I3,M5,E9,P5);(A3,I3,M5,E9,P6);(A3,I3,M5,E9,P7);(A3,I3,M5,E9,P8);(A3,I3,M5,E9,P9);(A3,I3,M5,E9,P10);(A3,I3,M5,E9,P11);(A3,I3,M5,E9,P12);(A3,I3,M5,E9,P13);(A3,I3,M5,E9,P14);(A3,I3,M5,E9,P15);(A3,I3,M5,E9,P16);(A3,I3,M5,E9,P17);(A3,I3,M5,E9,P18);(A3,I3,M5,E9,P19);(A3,I3,M5,E9,P20);(A3,I3,M6,E1,P1);(A3,I3,M6,E1,P2);(A3,I3,M6,E1,P3);(A3,I3,M6,E1,P4);(A3,I3,M6,E1,P5);(A3,I3,M6,E1,P6);(A3,I3,M6,E1,P7);(A3,I3,M6,E1,P8);(A3,I3,M6,E1,P9);(A3,I3,M6,E1,P10);(A3,I3,M6,E1,P11);(A3,I3,M6,E1,P12);(A3,I3,M6,E1,P13);(A3,I3,M6,E1,P14);(A3,I3,M6,E1,P15);(A3,I3,M6,E1,P16);(A3,I3,M6,E1,P17);(A3,I3,M6,E1,P18);(A3,I3,M6,E1,P19);(A3,I3,M6,E1,P20);(A3,I3,M6,E2,P1);(A3,I3,M6,E2,P2);(A3,I3,M6,E2,P3);(A3,I3,M6,E2,P4);(A3,I3,M6,E2,P5);(A3,I3,M6,E2,P6);(A3,I3,M6,E2,P7);(A3,I3,M6,E2,P8);(A3,I3,M6,E2,P9);(A3,I3,M6,E2,P10);(A3,I3,M6,E2,P11);(A3,I3,M6,E2,P12);(A3,I3,M6,E2,P13);(A3,I3,M6,E2,P14);(A3,I3,M6,E2,P15);(A3,I3,M6,E2,P16);(A3,I3,M6,E2,P17);(A3,I3,M6,E2,P18);(A3,I3,M6,E2,P19);(A3,I3,M6,E2,P20);(A3,I3,M6,E3,P1);(A3,I3,M6,E3,P2);(A3,I3,M6,E3,P3);(A3,I3,M6,E3,P4);(A3,I3,M6,E3,P5);(A3,I3,M6,E3,P6);(A3,I3,M6,E3,P7);(A3,I3,M6,E3,P8);(A3,I3,M6,E3,P9);(A3,I3,M6,E3,P10);(A3,I3,M6,E3,P11);(A3,I3,M6,E3,P12);(A3,I3,M6,E3,P13);(A3,I3,M6,E3,P14);(A3,I3,M6,E3,P15);(A3,I3,M6,E3,P16);(A3,I3,M6,E3,P17);(A3,I3,M6,E3,P18);(A3,I3,M6,E3,P19);(A3,I3,M6,E3,P20);(A3,I3,M6,E4,P1);(A3,I3,M6,E4,P2);(A3,I3,M6,E4,P3);(A3,I3,M6,E4,P4);(A3,I3,M6,E4,P5);(A3,I3,M6,E4,P6);(A3,I3,M6,E4,P7);(A3,I3,M6,E4,P8);(A3,I3,M6,E4,P9);(A3,I3,M6,E4,P10);(A3,I3,M6,E4,P11);(A3,I3,M6,E4,P12);(A3,I3,M6,E4,P13);(A3,I3,M6,E4,P14);(A3,I3,M6,E4,P15);(A3,I3,M6,E4,P16);(A3,I3,M6,E4,P17);(A3,I3,M6,E4,P18);(A3,I3,M6,E4,P19);(A3,I3,M6,E4,P20);(A3,I3,M6,E5,P1);(A3,I3,M6,E5,P2);(A3,I3,M6,E5,P3);(A3,I3,M6,E5,P4);(A3,I3,M6,E5,P5);(A3,I3,M6,E5,P6);(A3,I3,M6,E5,P7);(A3,I3,M6,E5,P8);(A3,I3,M6,E5,P9);(A3,I3,M6,E5,P10);(A3,I3,M6,E5,P11);(A3,I3,M6,E5,P12);(A3,I3,M6,E5,P13);(A3,I3,M6,E5,P14);(A3,I3,M6,E5,P15);(A3,I3,M6,E5,P16);(A3,I3,M6,E5,P17);(A3,I3,M6,E5,P18);(A3,I3,M6,E5,P19);(A3,I3,M6,E5,P20);(A3,I3,M6,E6,P1);(A3,I3,M6,E6,P2);(A3,I3,M6,E6,P3);(A3,I3,M6,E6,P4);(A3,I3,M6,E6,P5);(A3,I3,M6,E6,P6);(A3,I3,M6,E6,P7);(A3,I3,M6,E6,P8);(A3,I3,M6,E6,P9);(A3,I3,M6,E6,P10);(A3,I3,M6,E6,P11);(A3,I3,M6,E6,P12);(A3,I3,M6,E6,P13);(A3,I3,M6,E6,P14);(A3,I3,M6,E6,P15);(A3,I3,M6,E6,P16);(A3,I3,M6,E6,P17);(A3,I3,M6,E6,P18);(A3,I3,M6,E6,P19);(A3,I3,M6,E6,P20);(A3,I3,M6,E7,P1);(A3,I3,M6,E7,P2);(A3,I3,M6,E7,P3);(A3,I3,M6,E7,P4);(A3,I3,M6,E7,P5);(A3,I3,M6,E7,P6);(A3,I3,M6,E7,P7);(A3,I3,M6,E7,P8);(A3,I3,M6,E7,P9);(A3,I3,M6,E7,P10);(A3,I3,M6,E7,P11);(A3,I3,M6,E7,P12);(A3,I3,M6,E7,P13);(A3,I3,M6,E7,P14);(A3,I3,M6,E7,P15);(A3,I3,M6,E7,P16);(A3,I3,M6,E7,P17);(A3,I3,M6,E7,P18);(A3,I3,M6,E7,P19);(A3,I3,M6,E7,P20);(A3,I3,M6,E8,P1);(A3,I3,M6,E8,P2);(A3,I3,M6,E8,P3);(A3,I3,M6,E8,P4);(A3,I3,M6,E8,P5);(A3,I3,M6,E8,P6);(A3,I3,M6,E8,P7);(A3,I3,M6,E8,P8);(A3,I3,M6,E8,P9);(A3,I3,M6,E8,P10);(A3,I3,M6,E8,P11);(A3,I3,M6,E8,P12);(A3,I3,M6,E8,P13);(A3,I3,M6,E8,P14);(A3,I3,M6,E8,P15);(A3,I3,M6,E8,P16);(A3,I3,M6,E8,P17);(A3,I3,M6,E8,P18);(A3,I3,M6,E8,P19);(A3,I3,M6,E8,P20);(A3,I3,M6,E9,P1);(A3,I3,M6,E9,P2);(A3,I3,M6,E9,P3);(A3,I3,M6,E9,P4);(A3,I3,M6,E9,P5);(A3,I3,M6,E9,P6);(A3,I3,M6,E9,P7);(A3,I3,M6,E9,P8);(A3,I3,M6,E9,P9);(A3,I3,M6,E9,P10);(A3,I3,M6,E9,P11);(A3,I3,M6,E9,P12);(A3,I3,M6,E9,P13);(A3,I3,M6,E9,P14);(A3,I3,M6,E9,P15);(A3,I3,M6,E9,P16);(A3,I3,M6,E9,P17);(A3,I3,M6,E9,P18);(A3,I3,M6,E9,P19);(A3,I3,M6,E9,P20);(A4,I1,M1,E1,P1);(A4,I1,M1,E1,P2);(A4,I1,M1,E1,P3);(A4,I1,M1,E1,P4);(A4,I1,M1,E1,P5);(A4,I1,M1,E1,P6);(A4,I1,M1,E1,P7);(A4,I1,M1,E1,P8);(A4,I1,M1,E1,P9);(A4,I1,M1,E1,P10);(A4,I1,M1,E1,P11);(A4,I1,M1,E1,P12);(A4,I1,M1,E1,P13);(A4,I1,M1,E1,P14);(A4,I1,M1,E1,P15);(A4,I1,M1,E1,P16);(A4,I1,M1,E1,P17);(A4,I1,M1,E1,P18);(A4,I1,M1,E1,P19);(A4,I1,M1,E1,P20);(A4,I1,M1,E2,P1);(A4,I1,M1,E2,P2);(A4,I1,M1,E2,P3);(A4,I1,M1,E2,P4);(A4,I1,M1,E2,P5);(A4,I1,M1,E2,P6);(A4,I1,M1,E2,P7);(A4,I1,M1,E2,P8);(A4,I1,M1,E2,P9);(A4,I1,M1,E2,P10);(A4,I1,M1,E2,P11);(A4,I1,M1,E2,P12);(A4,I1,M1,E2,P13);(A4,I1,M1,E2,P14);(A4,I1,M1,E2,P15);(A4,I1,M1,E2,P16);(A4,I1,M1,E2,P17);(A4,I1,M1,E2,P18);(A4,I1,M1,E2,P19);(A4,I1,M1,E2,P20);(A4,I1,M1,E3,P1);(A4,I1,M1,E3,P2);(A4,I1,M1,E3,P3);(A4,I1,M1,E3,P4);(A4,I1,M1,E3,P5);(A4,I1,M1,E3,P6);(A4,I1,M1,E3,P7);(A4,I1,M1,E3,P8);(A4,I1,M1,E3,P9);(A4,I1,M1,E3,P10);(A4,I1,M1,E3,P11);(A4,I1,M1,E3,P12);(A4,I1,M1,E3,P13);(A4,I1,M1,E3,P14);(A4,I1,M1,E3,P15);(A4,I1,M1,E3,P16);(A4,I1,M1,E3,P17);(A4,I1,M1,E3,P18);(A4,I1,M1,E3,P19);(A4,I1,M1,E3,P20);(A4,I1,M1,E4,P1);(A4,I1,M1,E4,P2);(A4,I1,M1,E4,P3);(A4,I1,M1,E4,P4);(A4,I1,M1,E4,P5);(A4,I1,M1,E4,P6);(A4,I1,M1,E4,P7);(A4,I1,M1,E4,P8);(A4,I1,M1,E4,P9);(A4,I1,M1,E4,P10);(A4,I1,M1,E4,P11);(A4,I1,M1,E4,P12);(A4,I1,M1,E4,P13);(A4,I1,M1,E4,P14);(A4,I1,M1,E4,P15);(A4,I1,M1,E4,P16);(A4,I1,M1,E4,P17);(A4,I1,M1,E4,P18);(A4,I1,M1,E4,P19);(A4,I1,M1,E4,P20);(A4,I1,M1,E5,P1);(A4,I1,M1,E5,P2);(A4,I1,M1,E5,P3);(A4,I1,M1,E5,P4);(A4,I1,M1,E5,P5);(A4,I1,M1,E5,P6);(A4,I1,M1,E5,P7);(A4,I1,M1,E5,P8);(A4,I1,M1,E5,P9);(A4,I1,M1,E5,P10);(A4,I1,M1,E5,P11);(A4,I1,M1,E5,P12);(A4,I1,M1,E5,P13);(A4,I1,M1,E5,P14);(A4,I1,M1,E5,P15);(A4,I1,M1,E5,P16);(A4,I1,M1,E5,P17);(A4,I1,M1,E5,P18);(A4,I1,M1,E5,P19);(A4,I1,M1,E5,P20);(A4,I1,M1,E6,P1);(A4,I1,M1,E6,P2);(A4,I1,M1,E6,P3);(A4,I1,M1,E6,P4);(A4,I1,M1,E6,P5);(A4,I1,M1,E6,P6);(A4,I1,M1,E6,P7);(A4,I1,M1,E6,P8);(A4,I1,M1,E6,P9);(A4,I1,M1,E6,P10);(A4,I1,M1,E6,P11);(A4,I1,M1,E6,P12);(A4,I1,M1,E6,P13);(A4,I1,M1,E6,P14);(A4,I1,M1,E6,P15);(A4,I1,M1,E6,P16);(A4,I1,M1,E6,P17);(A4,I1,M1,E6,P18);(A4,I1,M1,E6,P19);(A4,I1,M1,E6,P20);(A4,I1,M1,E7,P1);(A4,I1,M1,E7,P2);(A4,I1,M1,E7,P3);(A4,I1,M1,E7,P4);(A4,I1,M1,E7,P5);(A4,I1,M1,E7,P6);(A4,I1,M1,E7,P7);(A4,I1,M1,E7,P8);(A4,I1,M1,E7,P9);(A4,I1,M1,E7,P10);(A4,I1,M1,E7,P11);(A4,I1,M1,E7,P12);(A4,I1,M1,E7,P13);(A4,I1,M1,E7,P14);(A4,I1,M1,E7,P15);(A4,I1,M1,E7,P16);(A4,I1,M1,E7,P17);(A4,I1,M1,E7,P18);(A4,I1,M1,E7,P19);(A4,I1,M1,E7,P20);(A4,I1,M1,E8,P1);(A4,I1,M1,E8,P2);(A4,I1,M1,E8,P3);(A4,I1,M1,E8,P4);(A4,I1,M1,E8,P5);(A4,I1,M1,E8,P6);(A4,I1,M1,E8,P7);(A4,I1,M1,E8,P8);(A4,I1,M1,E8,P9);(A4,I1,M1,E8,P10);(A4,I1,M1,E8,P11);(A4,I1,M1,E8,P12);(A4,I1,M1,E8,P13);(A4,I1,M1,E8,P14);(A4,I1,M1,E8,P15);(A4,I1,M1,E8,P16);(A4,I1,M1,E8,P17);(A4,I1,M1,E8,P18);(A4,I1,M1,E8,P19);(A4,I1,M1,E8,P20);(A4,I1,M1,E9,P1);(A4,I1,M1,E9,P2);(A4,I1,M1,E9,P3);(A4,I1,M1,E9,P4);(A4,I1,M1,E9,P5);(A4,I1,M1,E9,P6);(A4,I1,M1,E9,P7);(A4,I1,M1,E9,P8);(A4,I1,M1,E9,P9);(A4,I1,M1,E9,P10);(A4,I1,M1,E9,P11);(A4,I1,M1,E9,P12);(A4,I1,M1,E9,P13);(A4,I1,M1,E9,P14);(A4,I1,M1,E9,P15);(A4,I1,M1,E9,P16);(A4,I1,M1,E9,P17);(A4,I1,M1,E9,P18);(A4,I1,M1,E9,P19);(A4,I1,M1,E9,P20);(A4,I1,M2,E1,P1);(A4,I1,M2,E1,P2);(A4,I1,M2,E1,P3);(A4,I1,M2,E1,P4);(A4,I1,M2,E1,P5);(A4,I1,M2,E1,P6);(A4,I1,M2,E1,P7);(A4,I1,M2,E1,P8);(A4,I1,M2,E1,P9);(A4,I1,M2,E1,P10);(A4,I1,M2,E1,P11);(A4,I1,M2,E1,P12);(A4,I1,M2,E1,P13);(A4,I1,M2,E1,P14);(A4,I1,M2,E1,P15);(A4,I1,M2,E1,P16);(A4,I1,M2,E1,P17);(A4,I1,M2,E1,P18);(A4,I1,M2,E1,P19);(A4,I1,M2,E1,P20);(A4,I1,M2,E2,P1);(A4,I1,M2,E2,P2);(A4,I1,M2,E2,P3);(A4,I1,M2,E2,P4);(A4,I1,M2,E2,P5);(A4,I1,M2,E2,P6);(A4,I1,M2,E2,P7);(A4,I1,M2,E2,P8);(A4,I1,M2,E2,P9);(A4,I1,M2,E2,P10);(A4,I1,M2,E2,P11);(A4,I1,M2,E2,P12);(A4,I1,M2,E2,P13);(A4,I1,M2,E2,P14);(A4,I1,M2,E2,P15);(A4,I1,M2,E2,P16);(A4,I1,M2,E2,P17);(A4,I1,M2,E2,P18);(A4,I1,M2,E2,P19);(A4,I1,M2,E2,P20);(A4,I1,M2,E3,P1);(A4,I1,M2,E3,P2);(A4,I1,M2,E3,P3);(A4,I1,M2,E3,P4);(A4,I1,M2,E3,P5);(A4,I1,M2,E3,P6);(A4,I1,M2,E3,P7);(A4,I1,M2,E3,P8);(A4,I1,M2,E3,P9);(A4,I1,M2,E3,P10);(A4,I1,M2,E3,P11);(A4,I1,M2,E3,P12);(A4,I1,M2,E3,P13);(A4,I1,M2,E3,P14);(A4,I1,M2,E3,P15);(A4,I1,M2,E3,P16);(A4,I1,M2,E3,P17);(A4,I1,M2,E3,P18);(A4,I1,M2,E3,P19);(A4,I1,M2,E3,P20);(A4,I1,M2,E4,P1);(A4,I1,M2,E4,P2);(A4,I1,M2,E4,P3);(A4,I1,M2,E4,P4);(A4,I1,M2,E4,P5);(A4,I1,M2,E4,P6);(A4,I1,M2,E4,P7);(A4,I1,M2,E4,P8);(A4,I1,M2,E4,P9);(A4,I1,M2,E4,P10);(A4,I1,M2,E4,P11);(A4,I1,M2,E4,P12);(A4,I1,M2,E4,P13);(A4,I1,M2,E4,P14);(A4,I1,M2,E4,P15);(A4,I1,M2,E4,P16);(A4,I1,M2,E4,P17);(A4,I1,M2,E4,P18);(A4,I1,M2,E4,P19);(A4,I1,M2,E4,P20);(A4,I1,M2,E5,P1);(A4,I1,M2,E5,P2);(A4,I1,M2,E5,P3);(A4,I1,M2,E5,P4);(A4,I1,M2,E5,P5);(A4,I1,M2,E5,P6);(A4,I1,M2,E5,P7);(A4,I1,M2,E5,P8);(A4,I1,M2,E5,P9);(A4,I1,M2,E5,P10);(A4,I1,M2,E5,P11);(A4,I1,M2,E5,P12);(A4,I1,M2,E5,P13);(A4,I1,M2,E5,P14);(A4,I1,M2,E5,P15);(A4,I1,M2,E5,P16);(A4,I1,M2,E5,P17);(A4,I1,M2,E5,P18);(A4,I1,M2,E5,P19);(A4,I1,M2,E5,P20);(A4,I1,M2,E6,P1);(A4,I1,M2,E6,P2);(A4,I1,M2,E6,P3);(A4,I1,M2,E6,P4);(A4,I1,M2,E6,P5);(A4,I1,M2,E6,P6);(A4,I1,M2,E6,P7);(A4,I1,M2,E6,P8);(A4,I1,M2,E6,P9);(A4,I1,M2,E6,P10);(A4,I1,M2,E6,P11);(A4,I1,M2,E6,P12);(A4,I1,M2,E6,P13);(A4,I1,M2,E6,P14);(A4,I1,M2,E6,P15);(A4,I1,M2,E6,P16);(A4,I1,M2,E6,P17);(A4,I1,M2,E6,P18);(A4,I1,M2,E6,P19);(A4,I1,M2,E6,P20);(A4,I1,M2,E7,P1);(A4,I1,M2,E7,P2);(A4,I1,M2,E7,P3);(A4,I1,M2,E7,P4);(A4,I1,M2,E7,P5);(A4,I1,M2,E7,P6);(A4,I1,M2,E7,P7);(A4,I1,M2,E7,P8);(A4,I1,M2,E7,P9);(A4,I1,M2,E7,P10);(A4,I1,M2,E7,P11);(A4,I1,M2,E7,P12);(A4,I1,M2,E7,P13);(A4,I1,M2,E7,P14);(A4,I1,M2,E7,P15);(A4,I1,M2,E7,P16);(A4,I1,M2,E7,P17);(A4,I1,M2,E7,P18);(A4,I1,M2,E7,P19);(A4,I1,M2,E7,P20);(A4,I1,M2,E8,P1);(A4,I1,M2,E8,P2);(A4,I1,M2,E8,P3);(A4,I1,M2,E8,P4);(A4,I1,M2,E8,P5);(A4,I1,M2,E8,P6);(A4,I1,M2,E8,P7);(A4,I1,M2,E8,P8);(A4,I1,M2,E8,P9);(A4,I1,M2,E8,P10);(A4,I1,M2,E8,P11);(A4,I1,M2,E8,P12);(A4,I1,M2,E8,P13);(A4,I1,M2,E8,P14);(A4,I1,M2,E8,P15);(A4,I1,M2,E8,P16);(A4,I1,M2,E8,P17);(A4,I1,M2,E8,P18);(A4,I1,M2,E8,P19);(A4,I1,M2,E8,P20);(A4,I1,M2,E9,P1);(A4,I1,M2,E9,P2);(A4,I1,M2,E9,P3);(A4,I1,M2,E9,P4);(A4,I1,M2,E9,P5);(A4,I1,M2,E9,P6);(A4,I1,M2,E9,P7);(A4,I1,M2,E9,P8);(A4,I1,M2,E9,P9);(A4,I1,M2,E9,P10);(A4,I1,M2,E9,P11);(A4,I1,M2,E9,P12);(A4,I1,M2,E9,P13);(A4,I1,M2,E9,P14);(A4,I1,M2,E9,P15);(A4,I1,M2,E9,P16);(A4,I1,M2,E9,P17);(A4,I1,M2,E9,P18);(A4,I1,M2,E9,P19);(A4,I1,M2,E9,P20);(A4,I1,M3,E1,P1);(A4,I1,M3,E1,P2);(A4,I1,M3,E1,P3);(A4,I1,M3,E1,P4);(A4,I1,M3,E1,P5);(A4,I1,M3,E1,P6);(A4,I1,M3,E1,P7);(A4,I1,M3,E1,P8);(A4,I1,M3,E1,P9);(A4,I1,M3,E1,P10);(A4,I1,M3,E1,P11);(A4,I1,M3,E1,P12);(A4,I1,M3,E1,P13);(A4,I1,M3,E1,P14);(A4,I1,M3,E1,P15);(A4,I1,M3,E1,P16);(A4,I1,M3,E1,P17);(A4,I1,M3,E1,P18);(A4,I1,M3,E1,P19);(A4,I1,M3,E1,P20);(A4,I1,M3,E2,P1);(A4,I1,M3,E2,P2);(A4,I1,M3,E2,P3);(A4,I1,M3,E2,P4);(A4,I1,M3,E2,P5);(A4,I1,M3,E2,P6);(A4,I1,M3,E2,P7);(A4,I1,M3,E2,P8);(A4,I1,M3,E2,P9);(A4,I1,M3,E2,P10);(A4,I1,M3,E2,P11);(A4,I1,M3,E2,P12);(A4,I1,M3,E2,P13);(A4,I1,M3,E2,P14);(A4,I1,M3,E2,P15);(A4,I1,M3,E2,P16);(A4,I1,M3,E2,P17);(A4,I1,M3,E2,P18);(A4,I1,M3,E2,P19);(A4,I1,M3,E2,P20);(A4,I1,M3,E3,P1);(A4,I1,M3,E3,P2);(A4,I1,M3,E3,P3);(A4,I1,M3,E3,P4);(A4,I1,M3,E3,P5);(A4,I1,M3,E3,P6);(A4,I1,M3,E3,P7);(A4,I1,M3,E3,P8);(A4,I1,M3,E3,P9);(A4,I1,M3,E3,P10);(A4,I1,M3,E3,P11);(A4,I1,M3,E3,P12);(A4,I1,M3,E3,P13);(A4,I1,M3,E3,P14);(A4,I1,M3,E3,P15);(A4,I1,M3,E3,P16);(A4,I1,M3,E3,P17);(A4,I1,M3,E3,P18);(A4,I1,M3,E3,P19);(A4,I1,M3,E3,P20);(A4,I1,M3,E4,P1);(A4,I1,M3,E4,P2);(A4,I1,M3,E4,P3);(A4,I1,M3,E4,P4);(A4,I1,M3,E4,P5);(A4,I1,M3,E4,P6);(A4,I1,M3,E4,P7);(A4,I1,M3,E4,P8);(A4,I1,M3,E4,P9);(A4,I1,M3,E4,P10);(A4,I1,M3,E4,P11);(A4,I1,M3,E4,P12);(A4,I1,M3,E4,P13);(A4,I1,M3,E4,P14);(A4,I1,M3,E4,P15);(A4,I1,M3,E4,P16);(A4,I1,M3,E4,P17);(A4,I1,M3,E4,P18);(A4,I1,M3,E4,P19);(A4,I1,M3,E4,P20);(A4,I1,M3,E5,P1);(A4,I1,M3,E5,P2);(A4,I1,M3,E5,P3);(A4,I1,M3,E5,P4);(A4,I1,M3,E5,P5);(A4,I1,M3,E5,P6);(A4,I1,M3,E5,P7);(A4,I1,M3,E5,P8);(A4,I1,M3,E5,P9);(A4,I1,M3,E5,P10);(A4,I1,M3,E5,P11);(A4,I1,M3,E5,P12);(A4,I1,M3,E5,P13);(A4,I1,M3,E5,P14);(A4,I1,M3,E5,P15);(A4,I1,M3,E5,P16);(A4,I1,M3,E5,P17);(A4,I1,M3,E5,P18);(A4,I1,M3,E5,P19);(A4,I1,M3,E5,P20);(A4,I1,M3,E6,P1);(A4,I1,M3,E6,P2);(A4,I1,M3,E6,P3);(A4,I1,M3,E6,P4);(A4,I1,M3,E6,P5);(A4,I1,M3,E6,P6);(A4,I1,M3,E6,P7);(A4,I1,M3,E6,P8);(A4,I1,M3,E6,P9);(A4,I1,M3,E6,P10);(A4,I1,M3,E6,P11);(A4,I1,M3,E6,P12);(A4,I1,M3,E6,P13);(A4,I1,M3,E6,P14);(A4,I1,M3,E6,P15);(A4,I1,M3,E6,P16);(A4,I1,M3,E6,P17);(A4,I1,M3,E6,P18);(A4,I1,M3,E6,P19);(A4,I1,M3,E6,P20);(A4,I1,M3,E7,P1);(A4,I1,M3,E7,P2);(A4,I1,M3,E7,P3);(A4,I1,M3,E7,P4);(A4,I1,M3,E7,P5);(A4,I1,M3,E7,P6);(A4,I1,M3,E7,P7);(A4,I1,M3,E7,P8);(A4,I1,M3,E7,P9);(A4,I1,M3,E7,P10);(A4,I1,M3,E7,P11);(A4,I1,M3,E7,P12);(A4,I1,M3,E7,P13);(A4,I1,M3,E7,P14);(A4,I1,M3,E7,P15);(A4,I1,M3,E7,P16);(A4,I1,M3,E7,P17);(A4,I1,M3,E7,P18);(A4,I1,M3,E7,P19);(A4,I1,M3,E7,P20);(A4,I1,M3,E8,P1);(A4,I1,M3,E8,P2);(A4,I1,M3,E8,P3);(A4,I1,M3,E8,P4);(A4,I1,M3,E8,P5);(A4,I1,M3,E8,P6);(A4,I1,M3,E8,P7);(A4,I1,M3,E8,P8);(A4,I1,M3,E8,P9);(A4,I1,M3,E8,P10);(A4,I1,M3,E8,P11);(A4,I1,M3,E8,P12);(A4,I1,M3,E8,P13);(A4,I1,M3,E8,P14);(A4,I1,M3,E8,P15);(A4,I1,M3,E8,P16);(A4,I1,M3,E8,P17);(A4,I1,M3,E8,P18);(A4,I1,M3,E8,P19);(A4,I1,M3,E8,P20);(A4,I1,M3,E9,P1);(A4,I1,M3,E9,P2);(A4,I1,M3,E9,P3);(A4,I1,M3,E9,P4);(A4,I1,M3,E9,P5);(A4,I1,M3,E9,P6);(A4,I1,M3,E9,P7);(A4,I1,M3,E9,P8);(A4,I1,M3,E9,P9);(A4,I1,M3,E9,P10);(A4,I1,M3,E9,P11);(A4,I1,M3,E9,P12);(A4,I1,M3,E9,P13);(A4,I1,M3,E9,P14);(A4,I1,M3,E9,P15);(A4,I1,M3,E9,P16);(A4,I1,M3,E9,P17);(A4,I1,M3,E9,P18);(A4,I1,M3,E9,P19);(A4,I1,M3,E9,P20);(A4,I1,M4,E1,P1);(A4,I1,M4,E1,P2);(A4,I1,M4,E1,P3);(A4,I1,M4,E1,P4);(A4,I1,M4,E1,P5);(A4,I1,M4,E1,P6);(A4,I1,M4,E1,P7);(A4,I1,M4,E1,P8);(A4,I1,M4,E1,P9);(A4,I1,M4,E1,P10);(A4,I1,M4,E1,P11);(A4,I1,M4,E1,P12);(A4,I1,M4,E1,P13);(A4,I1,M4,E1,P14);(A4,I1,M4,E1,P15);(A4,I1,M4,E1,P16);(A4,I1,M4,E1,P17);(A4,I1,M4,E1,P18);(A4,I1,M4,E1,P19);(A4,I1,M4,E1,P20);(A4,I1,M4,E2,P1);(A4,I1,M4,E2,P2);(A4,I1,M4,E2,P3);(A4,I1,M4,E2,P4);(A4,I1,M4,E2,P5);(A4,I1,M4,E2,P6);(A4,I1,M4,E2,P7);(A4,I1,M4,E2,P8);(A4,I1,M4,E2,P9);(A4,I1,M4,E2,P10);(A4,I1,M4,E2,P11);(A4,I1,M4,E2,P12);(A4,I1,M4,E2,P13);(A4,I1,M4,E2,P14);(A4,I1,M4,E2,P15);(A4,I1,M4,E2,P16);(A4,I1,M4,E2,P17);(A4,I1,M4,E2,P18);(A4,I1,M4,E2,P19);(A4,I1,M4,E2,P20);(A4,I1,M4,E3,P1);(A4,I1,M4,E3,P2);(A4,I1,M4,E3,P3);(A4,I1,M4,E3,P4);(A4,I1,M4,E3,P5);(A4,I1,M4,E3,P6);(A4,I1,M4,E3,P7);(A4,I1,M4,E3,P8);(A4,I1,M4,E3,P9);(A4,I1,M4,E3,P10);(A4,I1,M4,E3,P11);(A4,I1,M4,E3,P12);(A4,I1,M4,E3,P13);(A4,I1,M4,E3,P14);(A4,I1,M4,E3,P15);(A4,I1,M4,E3,P16);(A4,I1,M4,E3,P17);(A4,I1,M4,E3,P18);(A4,I1,M4,E3,P19);(A4,I1,M4,E3,P20);(A4,I1,M4,E4,P1);(A4,I1,M4,E4,P2);(A4,I1,M4,E4,P3);(A4,I1,M4,E4,P4);(A4,I1,M4,E4,P5);(A4,I1,M4,E4,P6);(A4,I1,M4,E4,P7);(A4,I1,M4,E4,P8);(A4,I1,M4,E4,P9);(A4,I1,M4,E4,P10);(A4,I1,M4,E4,P11);(A4,I1,M4,E4,P12);(A4,I1,M4,E4,P13);(A4,I1,M4,E4,P14);(A4,I1,M4,E4,P15);(A4,I1,M4,E4,P16);(A4,I1,M4,E4,P17);(A4,I1,M4,E4,P18);(A4,I1,M4,E4,P19);(A4,I1,M4,E4,P20);(A4,I1,M4,E5,P1);(A4,I1,M4,E5,P2);(A4,I1,M4,E5,P3);(A4,I1,M4,E5,P4);(A4,I1,M4,E5,P5);(A4,I1,M4,E5,P6);(A4,I1,M4,E5,P7);(A4,I1,M4,E5,P8);(A4,I1,M4,E5,P9);(A4,I1,M4,E5,P10);(A4,I1,M4,E5,P11);(A4,I1,M4,E5,P12);(A4,I1,M4,E5,P13);(A4,I1,M4,E5,P14);(A4,I1,M4,E5,P15);(A4,I1,M4,E5,P16);(A4,I1,M4,E5,P17);(A4,I1,M4,E5,P18);(A4,I1,M4,E5,P19);(A4,I1,M4,E5,P20);(A4,I1,M4,E6,P1);(A4,I1,M4,E6,P2);(A4,I1,M4,E6,P3);(A4,I1,M4,E6,P4);(A4,I1,M4,E6,P5);(A4,I1,M4,E6,P6);(A4,I1,M4,E6,P7);(A4,I1,M4,E6,P8);(A4,I1,M4,E6,P9);(A4,I1,M4,E6,P10);(A4,I1,M4,E6,P11);(A4,I1,M4,E6,P12);(A4,I1,M4,E6,P13);(A4,I1,M4,E6,P14);(A4,I1,M4,E6,P15);(A4,I1,M4,E6,P16);(A4,I1,M4,E6,P17);(A4,I1,M4,E6,P18);(A4,I1,M4,E6,P19);(A4,I1,M4,E6,P20);(A4,I1,M4,E7,P1);(A4,I1,M4,E7,P2);(A4,I1,M4,E7,P3);(A4,I1,M4,E7,P4);(A4,I1,M4,E7,P5);(A4,I1,M4,E7,P6);(A4,I1,M4,E7,P7);(A4,I1,M4,E7,P8);(A4,I1,M4,E7,P9);(A4,I1,M4,E7,P10);(A4,I1,M4,E7,P11);(A4,I1,M4,E7,P12);(A4,I1,M4,E7,P13);(A4,I1,M4,E7,P14);(A4,I1,M4,E7,P15);(A4,I1,M4,E7,P16);(A4,I1,M4,E7,P17);(A4,I1,M4,E7,P18);(A4,I1,M4,E7,P19);(A4,I1,M4,E7,P20);(A4,I1,M4,E8,P1);(A4,I1,M4,E8,P2);(A4,I1,M4,E8,P3);(A4,I1,M4,E8,P4);(A4,I1,M4,E8,P5);(A4,I1,M4,E8,P6);(A4,I1,M4,E8,P7);(A4,I1,M4,E8,P8);(A4,I1,M4,E8,P9);(A4,I1,M4,E8,P10);(A4,I1,M4,E8,P11);(A4,I1,M4,E8,P12);(A4,I1,M4,E8,P13);(A4,I1,M4,E8,P14);(A4,I1,M4,E8,P15);(A4,I1,M4,E8,P16);(A4,I1,M4,E8,P17);(A4,I1,M4,E8,P18);(A4,I1,M4,E8,P19);(A4,I1,M4,E8,P20);(A4,I1,M4,E9,P1);(A4,I1,M4,E9,P2);(A4,I1,M4,E9,P3);(A4,I1,M4,E9,P4);(A4,I1,M4,E9,P5);(A4,I1,M4,E9,P6);(A4,I1,M4,E9,P7);(A4,I1,M4,E9,P8);(A4,I1,M4,E9,P9);(A4,I1,M4,E9,P10);(A4,I1,M4,E9,P11);(A4,I1,M4,E9,P12);(A4,I1,M4,E9,P13);(A4,I1,M4,E9,P14);(A4,I1,M4,E9,P15);(A4,I1,M4,E9,P16);(A4,I1,M4,E9,P17);(A4,I1,M4,E9,P18);(A4,I1,M4,E9,P19);(A4,I1,M4,E9,P20);(A4,I1,M5,E1,P1);(A4,I1,M5,E1,P2);(A4,I1,M5,E1,P3);(A4,I1,M5,E1,P4);(A4,I1,M5,E1,P5);(A4,I1,M5,E1,P6);(A4,I1,M5,E1,P7);(A4,I1,M5,E1,P8);(A4,I1,M5,E1,P9);(A4,I1,M5,E1,P10);(A4,I1,M5,E1,P11);(A4,I1,M5,E1,P12);(A4,I1,M5,E1,P13);(A4,I1,M5,E1,P14);(A4,I1,M5,E1,P15);(A4,I1,M5,E1,P16);(A4,I1,M5,E1,P17);(A4,I1,M5,E1,P18);(A4,I1,M5,E1,P19);(A4,I1,M5,E1,P20);(A4,I1,M5,E2,P1);(A4,I1,M5,E2,P2);(A4,I1,M5,E2,P3);(A4,I1,M5,E2,P4);(A4,I1,M5,E2,P5);(A4,I1,M5,E2,P6);(A4,I1,M5,E2,P7);(A4,I1,M5,E2,P8);(A4,I1,M5,E2,P9);(A4,I1,M5,E2,P10);(A4,I1,M5,E2,P11);(A4,I1,M5,E2,P12);(A4,I1,M5,E2,P13);(A4,I1,M5,E2,P14);(A4,I1,M5,E2,P15);(A4,I1,M5,E2,P16);(A4,I1,M5,E2,P17);(A4,I1,M5,E2,P18);(A4,I1,M5,E2,P19);(A4,I1,M5,E2,P20);(A4,I1,M5,E3,P1);(A4,I1,M5,E3,P2);(A4,I1,M5,E3,P3);(A4,I1,M5,E3,P4);(A4,I1,M5,E3,P5);(A4,I1,M5,E3,P6);(A4,I1,M5,E3,P7);(A4,I1,M5,E3,P8);(A4,I1,M5,E3,P9);(A4,I1,M5,E3,P10);(A4,I1,M5,E3,P11);(A4,I1,M5,E3,P12);(A4,I1,M5,E3,P13);(A4,I1,M5,E3,P14);(A4,I1,M5,E3,P15);(A4,I1,M5,E3,P16);(A4,I1,M5,E3,P17);(A4,I1,M5,E3,P18);(A4,I1,M5,E3,P19);(A4,I1,M5,E3,P20);(A4,I1,M5,E4,P1);(A4,I1,M5,E4,P2);(A4,I1,M5,E4,P3);(A4,I1,M5,E4,P4);(A4,I1,M5,E4,P5);(A4,I1,M5,E4,P6);(A4,I1,M5,E4,P7);(A4,I1,M5,E4,P8);(A4,I1,M5,E4,P9);(A4,I1,M5,E4,P10);(A4,I1,M5,E4,P11);(A4,I1,M5,E4,P12);(A4,I1,M5,E4,P13);(A4,I1,M5,E4,P14);(A4,I1,M5,E4,P15);(A4,I1,M5,E4,P16);(A4,I1,M5,E4,P17);(A4,I1,M5,E4,P18);(A4,I1,M5,E4,P19);(A4,I1,M5,E4,P20);(A4,I1,M5,E5,P1);(A4,I1,M5,E5,P2);(A4,I1,M5,E5,P3);(A4,I1,M5,E5,P4);(A4,I1,M5,E5,P5);(A4,I1,M5,E5,P6);(A4,I1,M5,E5,P7);(A4,I1,M5,E5,P8);(A4,I1,M5,E5,P9);(A4,I1,M5,E5,P10);(A4,I1,M5,E5,P11);(A4,I1,M5,E5,P12);(A4,I1,M5,E5,P13);(A4,I1,M5,E5,P14);(A4,I1,M5,E5,P15);(A4,I1,M5,E5,P16);(A4,I1,M5,E5,P17);(A4,I1,M5,E5,P18);(A4,I1,M5,E5,P19);(A4,I1,M5,E5,P20);(A4,I1,M5,E6,P1);(A4,I1,M5,E6,P2);(A4,I1,M5,E6,P3);(A4,I1,M5,E6,P4);(A4,I1,M5,E6,P5);(A4,I1,M5,E6,P6);(A4,I1,M5,E6,P7);(A4,I1,M5,E6,P8);(A4,I1,M5,E6,P9);(A4,I1,M5,E6,P10);(A4,I1,M5,E6,P11);(A4,I1,M5,E6,P12);(A4,I1,M5,E6,P13);(A4,I1,M5,E6,P14);(A4,I1,M5,E6,P15);(A4,I1,M5,E6,P16);(A4,I1,M5,E6,P17);(A4,I1,M5,E6,P18);(A4,I1,M5,E6,P19);(A4,I1,M5,E6,P20);(A4,I1,M5,E7,P1);(A4,I1,M5,E7,P2);(A4,I1,M5,E7,P3);(A4,I1,M5,E7,P4);(A4,I1,M5,E7,P5);(A4,I1,M5,E7,P6);(A4,I1,M5,E7,P7);(A4,I1,M5,E7,P8);(A4,I1,M5,E7,P9);(A4,I1,M5,E7,P10);(A4,I1,M5,E7,P11);(A4,I1,M5,E7,P12);(A4,I1,M5,E7,P13);(A4,I1,M5,E7,P14);(A4,I1,M5,E7,P15);(A4,I1,M5,E7,P16);(A4,I1,M5,E7,P17);(A4,I1,M5,E7,P18);(A4,I1,M5,E7,P19);(A4,I1,M5,E7,P20);(A4,I1,M5,E8,P1);(A4,I1,M5,E8,P2);(A4,I1,M5,E8,P3);(A4,I1,M5,E8,P4);(A4,I1,M5,E8,P5);(A4,I1,M5,E8,P6);(A4,I1,M5,E8,P7);(A4,I1,M5,E8,P8);(A4,I1,M5,E8,P9);(A4,I1,M5,E8,P10);(A4,I1,M5,E8,P11);(A4,I1,M5,E8,P12);(A4,I1,M5,E8,P13);(A4,I1,M5,E8,P14);(A4,I1,M5,E8,P15);(A4,I1,M5,E8,P16);(A4,I1,M5,E8,P17);(A4,I1,M5,E8,P18);(A4,I1,M5,E8,P19);(A4,I1,M5,E8,P20);(A4,I1,M5,E9,P1);(A4,I1,M5,E9,P2);(A4,I1,M5,E9,P3);(A4,I1,M5,E9,P4);(A4,I1,M5,E9,P5);(A4,I1,M5,E9,P6);(A4,I1,M5,E9,P7);(A4,I1,M5,E9,P8);(A4,I1,M5,E9,P9);(A4,I1,M5,E9,P10);(A4,I1,M5,E9,P11);(A4,I1,M5,E9,P12);(A4,I1,M5,E9,P13);(A4,I1,M5,E9,P14);(A4,I1,M5,E9,P15);(A4,I1,M5,E9,P16);(A4,I1,M5,E9,P17);(A4,I1,M5,E9,P18);(A4,I1,M5,E9,P19);(A4,I1,M5,E9,P20);(A4,I1,M6,E1,P1);(A4,I1,M6,E1,P2);(A4,I1,M6,E1,P3);(A4,I1,M6,E1,P4);(A4,I1,M6,E1,P5);(A4,I1,M6,E1,P6);(A4,I1,M6,E1,P7);(A4,I1,M6,E1,P8);(A4,I1,M6,E1,P9);(A4,I1,M6,E1,P10);(A4,I1,M6,E1,P11);(A4,I1,M6,E1,P12);(A4,I1,M6,E1,P13);(A4,I1,M6,E1,P14);(A4,I1,M6,E1,P15);(A4,I1,M6,E1,P16);(A4,I1,M6,E1,P17);(A4,I1,M6,E1,P18);(A4,I1,M6,E1,P19);(A4,I1,M6,E1,P20);(A4,I1,M6,E2,P1);(A4,I1,M6,E2,P2);(A4,I1,M6,E2,P3);(A4,I1,M6,E2,P4);(A4,I1,M6,E2,P5);(A4,I1,M6,E2,P6);(A4,I1,M6,E2,P7);(A4,I1,M6,E2,P8);(A4,I1,M6,E2,P9);(A4,I1,M6,E2,P10);(A4,I1,M6,E2,P11);(A4,I1,M6,E2,P12);(A4,I1,M6,E2,P13);(A4,I1,M6,E2,P14);(A4,I1,M6,E2,P15);(A4,I1,M6,E2,P16);(A4,I1,M6,E2,P17);(A4,I1,M6,E2,P18);(A4,I1,M6,E2,P19);(A4,I1,M6,E2,P20);(A4,I1,M6,E3,P1);(A4,I1,M6,E3,P2);(A4,I1,M6,E3,P3);(A4,I1,M6,E3,P4);(A4,I1,M6,E3,P5);(A4,I1,M6,E3,P6);(A4,I1,M6,E3,P7);(A4,I1,M6,E3,P8);(A4,I1,M6,E3,P9);(A4,I1,M6,E3,P10);(A4,I1,M6,E3,P11);(A4,I1,M6,E3,P12);(A4,I1,M6,E3,P13);(A4,I1,M6,E3,P14);(A4,I1,M6,E3,P15);(A4,I1,M6,E3,P16);(A4,I1,M6,E3,P17);(A4,I1,M6,E3,P18);(A4,I1,M6,E3,P19);(A4,I1,M6,E3,P20);(A4,I1,M6,E4,P1);(A4,I1,M6,E4,P2);(A4,I1,M6,E4,P3);(A4,I1,M6,E4,P4);(A4,I1,M6,E4,P5);(A4,I1,M6,E4,P6);(A4,I1,M6,E4,P7);(A4,I1,M6,E4,P8);(A4,I1,M6,E4,P9);(A4,I1,M6,E4,P10);(A4,I1,M6,E4,P11);(A4,I1,M6,E4,P12);(A4,I1,M6,E4,P13);(A4,I1,M6,E4,P14);(A4,I1,M6,E4,P15);(A4,I1,M6,E4,P16);(A4,I1,M6,E4,P17);(A4,I1,M6,E4,P18);(A4,I1,M6,E4,P19);(A4,I1,M6,E4,P20);(A4,I1,M6,E5,P1);(A4,I1,M6,E5,P2);(A4,I1,M6,E5,P3);(A4,I1,M6,E5,P4);(A4,I1,M6,E5,P5);(A4,I1,M6,E5,P6);(A4,I1,M6,E5,P7);(A4,I1,M6,E5,P8);(A4,I1,M6,E5,P9);(A4,I1,M6,E5,P10);(A4,I1,M6,E5,P11);(A4,I1,M6,E5,P12);(A4,I1,M6,E5,P13);(A4,I1,M6,E5,P14);(A4,I1,M6,E5,P15);(A4,I1,M6,E5,P16);(A4,I1,M6,E5,P17);(A4,I1,M6,E5,P18);(A4,I1,M6,E5,P19);(A4,I1,M6,E5,P20);(A4,I1,M6,E6,P1);(A4,I1,M6,E6,P2);(A4,I1,M6,E6,P3);(A4,I1,M6,E6,P4);(A4,I1,M6,E6,P5);(A4,I1,M6,E6,P6);(A4,I1,M6,E6,P7);(A4,I1,M6,E6,P8);(A4,I1,M6,E6,P9);(A4,I1,M6,E6,P10);(A4,I1,M6,E6,P11);(A4,I1,M6,E6,P12);(A4,I1,M6,E6,P13);(A4,I1,M6,E6,P14);(A4,I1,M6,E6,P15);(A4,I1,M6,E6,P16);(A4,I1,M6,E6,P17);(A4,I1,M6,E6,P18);(A4,I1,M6,E6,P19);(A4,I1,M6,E6,P20);(A4,I1,M6,E7,P1);(A4,I1,M6,E7,P2);(A4,I1,M6,E7,P3);(A4,I1,M6,E7,P4);(A4,I1,M6,E7,P5);(A4,I1,M6,E7,P6);(A4,I1,M6,E7,P7);(A4,I1,M6,E7,P8);(A4,I1,M6,E7,P9);(A4,I1,M6,E7,P10);(A4,I1,M6,E7,P11);(A4,I1,M6,E7,P12);(A4,I1,M6,E7,P13);(A4,I1,M6,E7,P14);(A4,I1,M6,E7,P15);(A4,I1,M6,E7,P16);(A4,I1,M6,E7,P17);(A4,I1,M6,E7,P18);(A4,I1,M6,E7,P19);(A4,I1,M6,E7,P20);(A4,I1,M6,E8,P1);(A4,I1,M6,E8,P2);(A4,I1,M6,E8,P3);(A4,I1,M6,E8,P4);(A4,I1,M6,E8,P5);(A4,I1,M6,E8,P6);(A4,I1,M6,E8,P7);(A4,I1,M6,E8,P8);(A4,I1,M6,E8,P9);(A4,I1,M6,E8,P10);(A4,I1,M6,E8,P11);(A4,I1,M6,E8,P12);(A4,I1,M6,E8,P13);(A4,I1,M6,E8,P14);(A4,I1,M6,E8,P15);(A4,I1,M6,E8,P16);(A4,I1,M6,E8,P17);(A4,I1,M6,E8,P18);(A4,I1,M6,E8,P19);(A4,I1,M6,E8,P20);(A4,I1,M6,E9,P1);(A4,I1,M6,E9,P2);(A4,I1,M6,E9,P3);(A4,I1,M6,E9,P4);(A4,I1,M6,E9,P5);(A4,I1,M6,E9,P6);(A4,I1,M6,E9,P7);(A4,I1,M6,E9,P8);(A4,I1,M6,E9,P9);(A4,I1,M6,E9,P10);(A4,I1,M6,E9,P11);(A4,I1,M6,E9,P12);(A4,I1,M6,E9,P13);(A4,I1,M6,E9,P14);(A4,I1,M6,E9,P15);(A4,I1,M6,E9,P16);(A4,I1,M6,E9,P17);(A4,I1,M6,E9,P18);(A4,I1,M6,E9,P19);(A4,I1,M6,E9,P20);(A4,I2,M1,E1,P1);(A4,I2,M1,E1,P2);(A4,I2,M1,E1,P3);(A4,I2,M1,E1,P4);(A4,I2,M1,E1,P5);(A4,I2,M1,E1,P6);(A4,I2,M1,E1,P7);(A4,I2,M1,E1,P8);(A4,I2,M1,E1,P9);(A4,I2,M1,E1,P10);(A4,I2,M1,E1,P11);(A4,I2,M1,E1,P12);(A4,I2,M1,E1,P13);(A4,I2,M1,E1,P14);(A4,I2,M1,E1,P15);(A4,I2,M1,E1,P16);(A4,I2,M1,E1,P17);(A4,I2,M1,E1,P18);(A4,I2,M1,E1,P19);(A4,I2,M1,E1,P20);(A4,I2,M1,E2,P1);(A4,I2,M1,E2,P2);(A4,I2,M1,E2,P3);(A4,I2,M1,E2,P4);(A4,I2,M1,E2,P5);(A4,I2,M1,E2,P6);(A4,I2,M1,E2,P7);(A4,I2,M1,E2,P8);(A4,I2,M1,E2,P9);(A4,I2,M1,E2,P10);(A4,I2,M1,E2,P11);(A4,I2,M1,E2,P12);(A4,I2,M1,E2,P13);(A4,I2,M1,E2,P14);(A4,I2,M1,E2,P15);(A4,I2,M1,E2,P16);(A4,I2,M1,E2,P17);(A4,I2,M1,E2,P18);(A4,I2,M1,E2,P19);(A4,I2,M1,E2,P20);(A4,I2,M1,E3,P1);(A4,I2,M1,E3,P2);(A4,I2,M1,E3,P3);(A4,I2,M1,E3,P4);(A4,I2,M1,E3,P5);(A4,I2,M1,E3,P6);(A4,I2,M1,E3,P7);(A4,I2,M1,E3,P8);(A4,I2,M1,E3,P9);(A4,I2,M1,E3,P10);(A4,I2,M1,E3,P11);(A4,I2,M1,E3,P12);(A4,I2,M1,E3,P13);(A4,I2,M1,E3,P14);(A4,I2,M1,E3,P15);(A4,I2,M1,E3,P16);(A4,I2,M1,E3,P17);(A4,I2,M1,E3,P18);(A4,I2,M1,E3,P19);(A4,I2,M1,E3,P20);(A4,I2,M1,E4,P1);(A4,I2,M1,E4,P2);(A4,I2,M1,E4,P3);(A4,I2,M1,E4,P4);(A4,I2,M1,E4,P5);(A4,I2,M1,E4,P6);(A4,I2,M1,E4,P7);(A4,I2,M1,E4,P8);(A4,I2,M1,E4,P9);(A4,I2,M1,E4,P10);(A4,I2,M1,E4,P11);(A4,I2,M1,E4,P12);(A4,I2,M1,E4,P13);(A4,I2,M1,E4,P14);(A4,I2,M1,E4,P15);(A4,I2,M1,E4,P16);(A4,I2,M1,E4,P17);(A4,I2,M1,E4,P18);(A4,I2,M1,E4,P19);(A4,I2,M1,E4,P20);(A4,I2,M1,E5,P1);(A4,I2,M1,E5,P2);(A4,I2,M1,E5,P3);(A4,I2,M1,E5,P4);(A4,I2,M1,E5,P5);(A4,I2,M1,E5,P6);(A4,I2,M1,E5,P7);(A4,I2,M1,E5,P8);(A4,I2,M1,E5,P9);(A4,I2,M1,E5,P10);(A4,I2,M1,E5,P11);(A4,I2,M1,E5,P12);(A4,I2,M1,E5,P13);(A4,I2,M1,E5,P14);(A4,I2,M1,E5,P15);(A4,I2,M1,E5,P16);(A4,I2,M1,E5,P17);(A4,I2,M1,E5,P18);(A4,I2,M1,E5,P19);(A4,I2,M1,E5,P20);(A4,I2,M1,E6,P1);(A4,I2,M1,E6,P2);(A4,I2,M1,E6,P3);(A4,I2,M1,E6,P4);(A4,I2,M1,E6,P5);(A4,I2,M1,E6,P6);(A4,I2,M1,E6,P7);(A4,I2,M1,E6,P8);(A4,I2,M1,E6,P9);(A4,I2,M1,E6,P10);(A4,I2,M1,E6,P11);(A4,I2,M1,E6,P12);(A4,I2,M1,E6,P13);(A4,I2,M1,E6,P14);(A4,I2,M1,E6,P15);(A4,I2,M1,E6,P16);(A4,I2,M1,E6,P17);(A4,I2,M1,E6,P18);(A4,I2,M1,E6,P19);(A4,I2,M1,E6,P20);(A4,I2,M1,E7,P1);(A4,I2,M1,E7,P2);(A4,I2,M1,E7,P3);(A4,I2,M1,E7,P4);(A4,I2,M1,E7,P5);(A4,I2,M1,E7,P6);(A4,I2,M1,E7,P7);(A4,I2,M1,E7,P8);(A4,I2,M1,E7,P9);(A4,I2,M1,E7,P10);(A4,I2,M1,E7,P11);(A4,I2,M1,E7,P12);(A4,I2,M1,E7,P13);(A4,I2,M1,E7,P14);(A4,I2,M1,E7,P15);(A4,I2,M1,E7,P16);(A4,I2,M1,E7,P17);(A4,I2,M1,E7,P18);(A4,I2,M1,E7,P19);(A4,I2,M1,E7,P20);(A4,I2,M1,E8,P1);(A4,I2,M1,E8,P2);(A4,I2,M1,E8,P3);(A4,I2,M1,E8,P4);(A4,I2,M1,E8,P5);(A4,I2,M1,E8,P6);(A4,I2,M1,E8,P7);(A4,I2,M1,E8,P8);(A4,I2,M1,E8,P9);(A4,I2,M1,E8,P10);(A4,I2,M1,E8,P11);(A4,I2,M1,E8,P12);(A4,I2,M1,E8,P13);(A4,I2,M1,E8,P14);(A4,I2,M1,E8,P15);(A4,I2,M1,E8,P16);(A4,I2,M1,E8,P17);(A4,I2,M1,E8,P18);(A4,I2,M1,E8,P19);(A4,I2,M1,E8,P20);(A4,I2,M1,E9,P1);(A4,I2,M1,E9,P2);(A4,I2,M1,E9,P3);(A4,I2,M1,E9,P4);(A4,I2,M1,E9,P5);(A4,I2,M1,E9,P6);(A4,I2,M1,E9,P7);(A4,I2,M1,E9,P8);(A4,I2,M1,E9,P9);(A4,I2,M1,E9,P10);(A4,I2,M1,E9,P11);(A4,I2,M1,E9,P12);(A4,I2,M1,E9,P13);(A4,I2,M1,E9,P14);(A4,I2,M1,E9,P15);(A4,I2,M1,E9,P16);(A4,I2,M1,E9,P17);(A4,I2,M1,E9,P18);(A4,I2,M1,E9,P19);(A4,I2,M1,E9,P20);(A4,I2,M2,E1,P1);(A4,I2,M2,E1,P2);(A4,I2,M2,E1,P3);(A4,I2,M2,E1,P4);(A4,I2,M2,E1,P5);(A4,I2,M2,E1,P6);(A4,I2,M2,E1,P7);(A4,I2,M2,E1,P8);(A4,I2,M2,E1,P9);(A4,I2,M2,E1,P10);(A4,I2,M2,E1,P11);(A4,I2,M2,E1,P12);(A4,I2,M2,E1,P13);(A4,I2,M2,E1,P14);(A4,I2,M2,E1,P15);(A4,I2,M2,E1,P16);(A4,I2,M2,E1,P17);(A4,I2,M2,E1,P18);(A4,I2,M2,E1,P19);(A4,I2,M2,E1,P20);(A4,I2,M2,E2,P1);(A4,I2,M2,E2,P2);(A4,I2,M2,E2,P3);(A4,I2,M2,E2,P4);(A4,I2,M2,E2,P5);(A4,I2,M2,E2,P6);(A4,I2,M2,E2,P7);(A4,I2,M2,E2,P8);(A4,I2,M2,E2,P9);(A4,I2,M2,E2,P10);(A4,I2,M2,E2,P11);(A4,I2,M2,E2,P12);(A4,I2,M2,E2,P13);(A4,I2,M2,E2,P14);(A4,I2,M2,E2,P15);(A4,I2,M2,E2,P16);(A4,I2,M2,E2,P17);(A4,I2,M2,E2,P18);(A4,I2,M2,E2,P19);(A4,I2,M2,E2,P20);(A4,I2,M2,E3,P1);(A4,I2,M2,E3,P2);(A4,I2,M2,E3,P3);(A4,I2,M2,E3,P4);(A4,I2,M2,E3,P5);(A4,I2,M2,E3,P6);(A4,I2,M2,E3,P7);(A4,I2,M2,E3,P8);(A4,I2,M2,E3,P9);(A4,I2,M2,E3,P10);(A4,I2,M2,E3,P11);(A4,I2,M2,E3,P12);(A4,I2,M2,E3,P13);(A4,I2,M2,E3,P14);(A4,I2,M2,E3,P15);(A4,I2,M2,E3,P16);(A4,I2,M2,E3,P17);(A4,I2,M2,E3,P18);(A4,I2,M2,E3,P19);(A4,I2,M2,E3,P20);(A4,I2,M2,E4,P1);(A4,I2,M2,E4,P2);(A4,I2,M2,E4,P3);(A4,I2,M2,E4,P4);(A4,I2,M2,E4,P5);(A4,I2,M2,E4,P6);(A4,I2,M2,E4,P7);(A4,I2,M2,E4,P8);(A4,I2,M2,E4,P9);(A4,I2,M2,E4,P10);(A4,I2,M2,E4,P11);(A4,I2,M2,E4,P12);(A4,I2,M2,E4,P13);(A4,I2,M2,E4,P14);(A4,I2,M2,E4,P15);(A4,I2,M2,E4,P16);(A4,I2,M2,E4,P17);(A4,I2,M2,E4,P18);(A4,I2,M2,E4,P19);(A4,I2,M2,E4,P20);(A4,I2,M2,E5,P1);(A4,I2,M2,E5,P2);(A4,I2,M2,E5,P3);(A4,I2,M2,E5,P4);(A4,I2,M2,E5,P5);(A4,I2,M2,E5,P6);(A4,I2,M2,E5,P7);(A4,I2,M2,E5,P8);(A4,I2,M2,E5,P9);(A4,I2,M2,E5,P10);(A4,I2,M2,E5,P11);(A4,I2,M2,E5,P12);(A4,I2,M2,E5,P13);(A4,I2,M2,E5,P14);(A4,I2,M2,E5,P15);(A4,I2,M2,E5,P16);(A4,I2,M2,E5,P17);(A4,I2,M2,E5,P18);(A4,I2,M2,E5,P19);(A4,I2,M2,E5,P20);(A4,I2,M2,E6,P1);(A4,I2,M2,E6,P2);(A4,I2,M2,E6,P3);(A4,I2,M2,E6,P4);(A4,I2,M2,E6,P5);(A4,I2,M2,E6,P6);(A4,I2,M2,E6,P7);(A4,I2,M2,E6,P8);(A4,I2,M2,E6,P9);(A4,I2,M2,E6,P10);(A4,I2,M2,E6,P11);(A4,I2,M2,E6,P12);(A4,I2,M2,E6,P13);(A4,I2,M2,E6,P14);(A4,I2,M2,E6,P15);(A4,I2,M2,E6,P16);(A4,I2,M2,E6,P17);(A4,I2,M2,E6,P18);(A4,I2,M2,E6,P19);(A4,I2,M2,E6,P20);(A4,I2,M2,E7,P1);(A4,I2,M2,E7,P2);(A4,I2,M2,E7,P3);(A4,I2,M2,E7,P4);(A4,I2,M2,E7,P5);(A4,I2,M2,E7,P6);(A4,I2,M2,E7,P7);(A4,I2,M2,E7,P8);(A4,I2,M2,E7,P9);(A4,I2,M2,E7,P10);(A4,I2,M2,E7,P11);(A4,I2,M2,E7,P12);(A4,I2,M2,E7,P13);(A4,I2,M2,E7,P14);(A4,I2,M2,E7,P15);(A4,I2,M2,E7,P16);(A4,I2,M2,E7,P17);(A4,I2,M2,E7,P18);(A4,I2,M2,E7,P19);(A4,I2,M2,E7,P20);(A4,I2,M2,E8,P1);(A4,I2,M2,E8,P2);(A4,I2,M2,E8,P3);(A4,I2,M2,E8,P4);(A4,I2,M2,E8,P5);(A4,I2,M2,E8,P6);(A4,I2,M2,E8,P7);(A4,I2,M2,E8,P8);(A4,I2,M2,E8,P9);(A4,I2,M2,E8,P10);(A4,I2,M2,E8,P11);(A4,I2,M2,E8,P12);(A4,I2,M2,E8,P13);(A4,I2,M2,E8,P14);(A4,I2,M2,E8,P15);(A4,I2,M2,E8,P16);(A4,I2,M2,E8,P17);(A4,I2,M2,E8,P18);(A4,I2,M2,E8,P19);(A4,I2,M2,E8,P20);(A4,I2,M2,E9,P1);(A4,I2,M2,E9,P2);(A4,I2,M2,E9,P3);(A4,I2,M2,E9,P4);(A4,I2,M2,E9,P5);(A4,I2,M2,E9,P6);(A4,I2,M2,E9,P7);(A4,I2,M2,E9,P8);(A4,I2,M2,E9,P9);(A4,I2,M2,E9,P10);(A4,I2,M2,E9,P11);(A4,I2,M2,E9,P12);(A4,I2,M2,E9,P13);(A4,I2,M2,E9,P14);(A4,I2,M2,E9,P15);(A4,I2,M2,E9,P16);(A4,I2,M2,E9,P17);(A4,I2,M2,E9,P18);(A4,I2,M2,E9,P19);(A4,I2,M2,E9,P20);(A4,I2,M3,E1,P1);(A4,I2,M3,E1,P2);(A4,I2,M3,E1,P3);(A4,I2,M3,E1,P4);(A4,I2,M3,E1,P5);(A4,I2,M3,E1,P6);(A4,I2,M3,E1,P7);(A4,I2,M3,E1,P8);(A4,I2,M3,E1,P9);(A4,I2,M3,E1,P10);(A4,I2,M3,E1,P11);(A4,I2,M3,E1,P12);(A4,I2,M3,E1,P13);(A4,I2,M3,E1,P14);(A4,I2,M3,E1,P15);(A4,I2,M3,E1,P16);(A4,I2,M3,E1,P17);(A4,I2,M3,E1,P18);(A4,I2,M3,E1,P19);(A4,I2,M3,E1,P20);(A4,I2,M3,E2,P1);(A4,I2,M3,E2,P2);(A4,I2,M3,E2,P3);(A4,I2,M3,E2,P4);(A4,I2,M3,E2,P5);(A4,I2,M3,E2,P6);(A4,I2,M3,E2,P7);(A4,I2,M3,E2,P8);(A4,I2,M3,E2,P9);(A4,I2,M3,E2,P10);(A4,I2,M3,E2,P11);(A4,I2,M3,E2,P12);(A4,I2,M3,E2,P13);(A4,I2,M3,E2,P14);(A4,I2,M3,E2,P15);(A4,I2,M3,E2,P16);(A4,I2,M3,E2,P17);(A4,I2,M3,E2,P18);(A4,I2,M3,E2,P19);(A4,I2,M3,E2,P20);(A4,I2,M3,E3,P1);(A4,I2,M3,E3,P2);(A4,I2,M3,E3,P3);(A4,I2,M3,E3,P4);(A4,I2,M3,E3,P5);(A4,I2,M3,E3,P6);(A4,I2,M3,E3,P7);(A4,I2,M3,E3,P8);(A4,I2,M3,E3,P9);(A4,I2,M3,E3,P10);(A4,I2,M3,E3,P11);(A4,I2,M3,E3,P12);(A4,I2,M3,E3,P13);(A4,I2,M3,E3,P14);(A4,I2,M3,E3,P15);(A4,I2,M3,E3,P16);(A4,I2,M3,E3,P17);(A4,I2,M3,E3,P18);(A4,I2,M3,E3,P19);(A4,I2,M3,E3,P20);(A4,I2,M3,E4,P1);(A4,I2,M3,E4,P2);(A4,I2,M3,E4,P3);(A4,I2,M3,E4,P4);(A4,I2,M3,E4,P5);(A4,I2,M3,E4,P6);(A4,I2,M3,E4,P7);(A4,I2,M3,E4,P8);(A4,I2,M3,E4,P9);(A4,I2,M3,E4,P10);(A4,I2,M3,E4,P11);(A4,I2,M3,E4,P12);(A4,I2,M3,E4,P13);(A4,I2,M3,E4,P14);(A4,I2,M3,E4,P15);(A4,I2,M3,E4,P16);(A4,I2,M3,E4,P17);(A4,I2,M3,E4,P18);(A4,I2,M3,E4,P19);(A4,I2,M3,E4,P20);(A4,I2,M3,E5,P1);(A4,I2,M3,E5,P2);(A4,I2,M3,E5,P3);(A4,I2,M3,E5,P4);(A4,I2,M3,E5,P5);(A4,I2,M3,E5,P6);(A4,I2,M3,E5,P7);(A4,I2,M3,E5,P8);(A4,I2,M3,E5,P9);(A4,I2,M3,E5,P10);(A4,I2,M3,E5,P11);(A4,I2,M3,E5,P12);(A4,I2,M3,E5,P13);(A4,I2,M3,E5,P14);(A4,I2,M3,E5,P15);(A4,I2,M3,E5,P16);(A4,I2,M3,E5,P17);(A4,I2,M3,E5,P18);(A4,I2,M3,E5,P19);(A4,I2,M3,E5,P20);(A4,I2,M3,E6,P1);(A4,I2,M3,E6,P2);(A4,I2,M3,E6,P3);(A4,I2,M3,E6,P4);(A4,I2,M3,E6,P5);(A4,I2,M3,E6,P6);(A4,I2,M3,E6,P7);(A4,I2,M3,E6,P8);(A4,I2,M3,E6,P9);(A4,I2,M3,E6,P10);(A4,I2,M3,E6,P11);(A4,I2,M3,E6,P12);(A4,I2,M3,E6,P13);(A4,I2,M3,E6,P14);(A4,I2,M3,E6,P15);(A4,I2,M3,E6,P16);(A4,I2,M3,E6,P17);(A4,I2,M3,E6,P18);(A4,I2,M3,E6,P19);(A4,I2,M3,E6,P20);(A4,I2,M3,E7,P1);(A4,I2,M3,E7,P2);(A4,I2,M3,E7,P3);(A4,I2,M3,E7,P4);(A4,I2,M3,E7,P5);(A4,I2,M3,E7,P6);(A4,I2,M3,E7,P7);(A4,I2,M3,E7,P8);(A4,I2,M3,E7,P9);(A4,I2,M3,E7,P10);(A4,I2,M3,E7,P11);(A4,I2,M3,E7,P12);(A4,I2,M3,E7,P13);(A4,I2,M3,E7,P14);(A4,I2,M3,E7,P15);(A4,I2,M3,E7,P16);(A4,I2,M3,E7,P17);(A4,I2,M3,E7,P18);(A4,I2,M3,E7,P19);(A4,I2,M3,E7,P20);(A4,I2,M3,E8,P1);(A4,I2,M3,E8,P2);(A4,I2,M3,E8,P3);(A4,I2,M3,E8,P4);(A4,I2,M3,E8,P5);(A4,I2,M3,E8,P6);(A4,I2,M3,E8,P7);(A4,I2,M3,E8,P8);(A4,I2,M3,E8,P9);(A4,I2,M3,E8,P10);(A4,I2,M3,E8,P11);(A4,I2,M3,E8,P12);(A4,I2,M3,E8,P13);(A4,I2,M3,E8,P14);(A4,I2,M3,E8,P15);(A4,I2,M3,E8,P16);(A4,I2,M3,E8,P17);(A4,I2,M3,E8,P18);(A4,I2,M3,E8,P19);(A4,I2,M3,E8,P20);(A4,I2,M3,E9,P1);(A4,I2,M3,E9,P2);(A4,I2,M3,E9,P3);(A4,I2,M3,E9,P4);(A4,I2,M3,E9,P5);(A4,I2,M3,E9,P6);(A4,I2,M3,E9,P7);(A4,I2,M3,E9,P8);(A4,I2,M3,E9,P9);(A4,I2,M3,E9,P10);(A4,I2,M3,E9,P11);(A4,I2,M3,E9,P12);(A4,I2,M3,E9,P13);(A4,I2,M3,E9,P14);(A4,I2,M3,E9,P15);(A4,I2,M3,E9,P16);(A4,I2,M3,E9,P17);(A4,I2,M3,E9,P18);(A4,I2,M3,E9,P19);(A4,I2,M3,E9,P20);(A4,I2,M4,E1,P1);(A4,I2,M4,E1,P2);(A4,I2,M4,E1,P3);(A4,I2,M4,E1,P4);(A4,I2,M4,E1,P5);(A4,I2,M4,E1,P6);(A4,I2,M4,E1,P7);(A4,I2,M4,E1,P8);(A4,I2,M4,E1,P9);(A4,I2,M4,E1,P10);(A4,I2,M4,E1,P11);(A4,I2,M4,E1,P12);(A4,I2,M4,E1,P13);(A4,I2,M4,E1,P14);(A4,I2,M4,E1,P15);(A4,I2,M4,E1,P16);(A4,I2,M4,E1,P17);(A4,I2,M4,E1,P18);(A4,I2,M4,E1,P19);(A4,I2,M4,E1,P20);(A4,I2,M4,E2,P1);(A4,I2,M4,E2,P2);(A4,I2,M4,E2,P3);(A4,I2,M4,E2,P4);(A4,I2,M4,E2,P5);(A4,I2,M4,E2,P6);(A4,I2,M4,E2,P7);(A4,I2,M4,E2,P8);(A4,I2,M4,E2,P9);(A4,I2,M4,E2,P10);(A4,I2,M4,E2,P11);(A4,I2,M4,E2,P12);(A4,I2,M4,E2,P13);(A4,I2,M4,E2,P14);(A4,I2,M4,E2,P15);(A4,I2,M4,E2,P16);(A4,I2,M4,E2,P17);(A4,I2,M4,E2,P18);(A4,I2,M4,E2,P19);(A4,I2,M4,E2,P20);(A4,I2,M4,E3,P1);(A4,I2,M4,E3,P2);(A4,I2,M4,E3,P3);(A4,I2,M4,E3,P4);(A4,I2,M4,E3,P5);(A4,I2,M4,E3,P6);(A4,I2,M4,E3,P7);(A4,I2,M4,E3,P8);(A4,I2,M4,E3,P9);(A4,I2,M4,E3,P10);(A4,I2,M4,E3,P11);(A4,I2,M4,E3,P12);(A4,I2,M4,E3,P13);(A4,I2,M4,E3,P14);(A4,I2,M4,E3,P15);(A4,I2,M4,E3,P16);(A4,I2,M4,E3,P17);(A4,I2,M4,E3,P18);(A4,I2,M4,E3,P19);(A4,I2,M4,E3,P20);(A4,I2,M4,E4,P1);(A4,I2,M4,E4,P2);(A4,I2,M4,E4,P3);(A4,I2,M4,E4,P4);(A4,I2,M4,E4,P5);(A4,I2,M4,E4,P6);(A4,I2,M4,E4,P7);(A4,I2,M4,E4,P8);(A4,I2,M4,E4,P9);(A4,I2,M4,E4,P10);(A4,I2,M4,E4,P11);(A4,I2,M4,E4,P12);(A4,I2,M4,E4,P13);(A4,I2,M4,E4,P14);(A4,I2,M4,E4,P15);(A4,I2,M4,E4,P16);(A4,I2,M4,E4,P17);(A4,I2,M4,E4,P18);(A4,I2,M4,E4,P19);(A4,I2,M4,E4,P20);(A4,I2,M4,E5,P1);(A4,I2,M4,E5,P2);(A4,I2,M4,E5,P3);(A4,I2,M4,E5,P4);(A4,I2,M4,E5,P5);(A4,I2,M4,E5,P6);(A4,I2,M4,E5,P7);(A4,I2,M4,E5,P8);(A4,I2,M4,E5,P9);(A4,I2,M4,E5,P10);(A4,I2,M4,E5,P11);(A4,I2,M4,E5,P12);(A4,I2,M4,E5,P13);(A4,I2,M4,E5,P14);(A4,I2,M4,E5,P15);(A4,I2,M4,E5,P16);(A4,I2,M4,E5,P17);(A4,I2,M4,E5,P18);(A4,I2,M4,E5,P19);(A4,I2,M4,E5,P20);(A4,I2,M4,E6,P1);(A4,I2,M4,E6,P2);(A4,I2,M4,E6,P3);(A4,I2,M4,E6,P4);(A4,I2,M4,E6,P5);(A4,I2,M4,E6,P6);(A4,I2,M4,E6,P7);(A4,I2,M4,E6,P8);(A4,I2,M4,E6,P9);(A4,I2,M4,E6,P10);(A4,I2,M4,E6,P11);(A4,I2,M4,E6,P12);(A4,I2,M4,E6,P13);(A4,I2,M4,E6,P14);(A4,I2,M4,E6,P15);(A4,I2,M4,E6,P16);(A4,I2,M4,E6,P17);(A4,I2,M4,E6,P18);(A4,I2,M4,E6,P19);(A4,I2,M4,E6,P20);(A4,I2,M4,E7,P1);(A4,I2,M4,E7,P2);(A4,I2,M4,E7,P3);(A4,I2,M4,E7,P4);(A4,I2,M4,E7,P5);(A4,I2,M4,E7,P6);(A4,I2,M4,E7,P7);(A4,I2,M4,E7,P8);(A4,I2,M4,E7,P9);(A4,I2,M4,E7,P10);(A4,I2,M4,E7,P11);(A4,I2,M4,E7,P12);(A4,I2,M4,E7,P13);(A4,I2,M4,E7,P14);(A4,I2,M4,E7,P15);(A4,I2,M4,E7,P16);(A4,I2,M4,E7,P17);(A4,I2,M4,E7,P18);(A4,I2,M4,E7,P19);(A4,I2,M4,E7,P20);(A4,I2,M4,E8,P1);(A4,I2,M4,E8,P2);(A4,I2,M4,E8,P3);(A4,I2,M4,E8,P4);(A4,I2,M4,E8,P5);(A4,I2,M4,E8,P6);(A4,I2,M4,E8,P7);(A4,I2,M4,E8,P8);(A4,I2,M4,E8,P9);(A4,I2,M4,E8,P10);(A4,I2,M4,E8,P11);(A4,I2,M4,E8,P12);(A4,I2,M4,E8,P13);(A4,I2,M4,E8,P14);(A4,I2,M4,E8,P15);(A4,I2,M4,E8,P16);(A4,I2,M4,E8,P17);(A4,I2,M4,E8,P18);(A4,I2,M4,E8,P19);(A4,I2,M4,E8,P20);(A4,I2,M4,E9,P1);(A4,I2,M4,E9,P2);(A4,I2,M4,E9,P3);(A4,I2,M4,E9,P4);(A4,I2,M4,E9,P5);(A4,I2,M4,E9,P6);(A4,I2,M4,E9,P7);(A4,I2,M4,E9,P8);(A4,I2,M4,E9,P9);(A4,I2,M4,E9,P10);(A4,I2,M4,E9,P11);(A4,I2,M4,E9,P12);(A4,I2,M4,E9,P13);(A4,I2,M4,E9,P14);(A4,I2,M4,E9,P15);(A4,I2,M4,E9,P16);(A4,I2,M4,E9,P17);(A4,I2,M4,E9,P18);(A4,I2,M4,E9,P19);(A4,I2,M4,E9,P20);(A4,I2,M5,E1,P1);(A4,I2,M5,E1,P2);(A4,I2,M5,E1,P3);(A4,I2,M5,E1,P4);(A4,I2,M5,E1,P5);(A4,I2,M5,E1,P6);(A4,I2,M5,E1,P7);(A4,I2,M5,E1,P8);(A4,I2,M5,E1,P9);(A4,I2,M5,E1,P10);(A4,I2,M5,E1,P11);(A4,I2,M5,E1,P12);(A4,I2,M5,E1,P13);(A4,I2,M5,E1,P14);(A4,I2,M5,E1,P15);(A4,I2,M5,E1,P16);(A4,I2,M5,E1,P17);(A4,I2,M5,E1,P18);(A4,I2,M5,E1,P19);(A4,I2,M5,E1,P20);(A4,I2,M5,E2,P1);(A4,I2,M5,E2,P2);(A4,I2,M5,E2,P3);(A4,I2,M5,E2,P4);(A4,I2,M5,E2,P5);(A4,I2,M5,E2,P6);(A4,I2,M5,E2,P7);(A4,I2,M5,E2,P8);(A4,I2,M5,E2,P9);(A4,I2,M5,E2,P10);(A4,I2,M5,E2,P11);(A4,I2,M5,E2,P12);(A4,I2,M5,E2,P13);(A4,I2,M5,E2,P14);(A4,I2,M5,E2,P15);(A4,I2,M5,E2,P16);(A4,I2,M5,E2,P17);(A4,I2,M5,E2,P18);(A4,I2,M5,E2,P19);(A4,I2,M5,E2,P20);(A4,I2,M5,E3,P1);(A4,I2,M5,E3,P2);(A4,I2,M5,E3,P3);(A4,I2,M5,E3,P4);(A4,I2,M5,E3,P5);(A4,I2,M5,E3,P6);(A4,I2,M5,E3,P7);(A4,I2,M5,E3,P8);(A4,I2,M5,E3,P9);(A4,I2,M5,E3,P10);(A4,I2,M5,E3,P11);(A4,I2,M5,E3,P12);(A4,I2,M5,E3,P13);(A4,I2,M5,E3,P14);(A4,I2,M5,E3,P15);(A4,I2,M5,E3,P16);(A4,I2,M5,E3,P17);(A4,I2,M5,E3,P18);(A4,I2,M5,E3,P19);(A4,I2,M5,E3,P20);(A4,I2,M5,E4,P1);(A4,I2,M5,E4,P2);(A4,I2,M5,E4,P3);(A4,I2,M5,E4,P4);(A4,I2,M5,E4,P5);(A4,I2,M5,E4,P6);(A4,I2,M5,E4,P7);(A4,I2,M5,E4,P8);(A4,I2,M5,E4,P9);(A4,I2,M5,E4,P10);(A4,I2,M5,E4,P11);(A4,I2,M5,E4,P12);(A4,I2,M5,E4,P13);(A4,I2,M5,E4,P14);(A4,I2,M5,E4,P15);(A4,I2,M5,E4,P16);(A4,I2,M5,E4,P17);(A4,I2,M5,E4,P18);(A4,I2,M5,E4,P19);(A4,I2,M5,E4,P20);(A4,I2,M5,E5,P1);(A4,I2,M5,E5,P2);(A4,I2,M5,E5,P3);(A4,I2,M5,E5,P4);(A4,I2,M5,E5,P5);(A4,I2,M5,E5,P6);(A4,I2,M5,E5,P7);(A4,I2,M5,E5,P8);(A4,I2,M5,E5,P9);(A4,I2,M5,E5,P10);(A4,I2,M5,E5,P11);(A4,I2,M5,E5,P12);(A4,I2,M5,E5,P13);(A4,I2,M5,E5,P14);(A4,I2,M5,E5,P15);(A4,I2,M5,E5,P16);(A4,I2,M5,E5,P17);(A4,I2,M5,E5,P18);(A4,I2,M5,E5,P19);(A4,I2,M5,E5,P20);(A4,I2,M5,E6,P1);(A4,I2,M5,E6,P2);(A4,I2,M5,E6,P3);(A4,I2,M5,E6,P4);(A4,I2,M5,E6,P5);(A4,I2,M5,E6,P6);(A4,I2,M5,E6,P7);(A4,I2,M5,E6,P8);(A4,I2,M5,E6,P9);(A4,I2,M5,E6,P10);(A4,I2,M5,E6,P11);(A4,I2,M5,E6,P12);(A4,I2,M5,E6,P13);(A4,I2,M5,E6,P14);(A4,I2,M5,E6,P15);(A4,I2,M5,E6,P16);(A4,I2,M5,E6,P17);(A4,I2,M5,E6,P18);(A4,I2,M5,E6,P19);(A4,I2,M5,E6,P20);(A4,I2,M5,E7,P1);(A4,I2,M5,E7,P2);(A4,I2,M5,E7,P3);(A4,I2,M5,E7,P4);(A4,I2,M5,E7,P5);(A4,I2,M5,E7,P6);(A4,I2,M5,E7,P7);(A4,I2,M5,E7,P8);(A4,I2,M5,E7,P9);(A4,I2,M5,E7,P10);(A4,I2,M5,E7,P11);(A4,I2,M5,E7,P12);(A4,I2,M5,E7,P13);(A4,I2,M5,E7,P14);(A4,I2,M5,E7,P15);(A4,I2,M5,E7,P16);(A4,I2,M5,E7,P17);(A4,I2,M5,E7,P18);(A4,I2,M5,E7,P19);(A4,I2,M5,E7,P20);(A4,I2,M5,E8,P1);(A4,I2,M5,E8,P2);(A4,I2,M5,E8,P3);(A4,I2,M5,E8,P4);(A4,I2,M5,E8,P5);(A4,I2,M5,E8,P6);(A4,I2,M5,E8,P7);(A4,I2,M5,E8,P8);(A4,I2,M5,E8,P9);(A4,I2,M5,E8,P10);(A4,I2,M5,E8,P11);(A4,I2,M5,E8,P12);(A4,I2,M5,E8,P13);(A4,I2,M5,E8,P14);(A4,I2,M5,E8,P15);(A4,I2,M5,E8,P16);(A4,I2,M5,E8,P17);(A4,I2,M5,E8,P18);(A4,I2,M5,E8,P19);(A4,I2,M5,E8,P20);(A4,I2,M5,E9,P1);(A4,I2,M5,E9,P2);(A4,I2,M5,E9,P3);(A4,I2,M5,E9,P4);(A4,I2,M5,E9,P5);(A4,I2,M5,E9,P6);(A4,I2,M5,E9,P7);(A4,I2,M5,E9,P8);(A4,I2,M5,E9,P9);(A4,I2,M5,E9,P10);(A4,I2,M5,E9,P11);(A4,I2,M5,E9,P12);(A4,I2,M5,E9,P13);(A4,I2,M5,E9,P14);(A4,I2,M5,E9,P15);(A4,I2,M5,E9,P16);(A4,I2,M5,E9,P17);(A4,I2,M5,E9,P18);(A4,I2,M5,E9,P19);(A4,I2,M5,E9,P20);(A4,I2,M6,E1,P1);(A4,I2,M6,E1,P2);(A4,I2,M6,E1,P3);(A4,I2,M6,E1,P4);(A4,I2,M6,E1,P5);(A4,I2,M6,E1,P6);(A4,I2,M6,E1,P7);(A4,I2,M6,E1,P8);(A4,I2,M6,E1,P9);(A4,I2,M6,E1,P10);(A4,I2,M6,E1,P11);(A4,I2,M6,E1,P12);(A4,I2,M6,E1,P13);(A4,I2,M6,E1,P14);(A4,I2,M6,E1,P15);(A4,I2,M6,E1,P16);(A4,I2,M6,E1,P17);(A4,I2,M6,E1,P18);(A4,I2,M6,E1,P19);(A4,I2,M6,E1,P20);(A4,I2,M6,E2,P1);(A4,I2,M6,E2,P2);(A4,I2,M6,E2,P3);(A4,I2,M6,E2,P4);(A4,I2,M6,E2,P5);(A4,I2,M6,E2,P6);(A4,I2,M6,E2,P7);(A4,I2,M6,E2,P8);(A4,I2,M6,E2,P9);(A4,I2,M6,E2,P10);(A4,I2,M6,E2,P11);(A4,I2,M6,E2,P12);(A4,I2,M6,E2,P13);(A4,I2,M6,E2,P14);(A4,I2,M6,E2,P15);(A4,I2,M6,E2,P16);(A4,I2,M6,E2,P17);(A4,I2,M6,E2,P18);(A4,I2,M6,E2,P19);(A4,I2,M6,E2,P20);(A4,I2,M6,E3,P1);(A4,I2,M6,E3,P2);(A4,I2,M6,E3,P3);(A4,I2,M6,E3,P4);(A4,I2,M6,E3,P5);(A4,I2,M6,E3,P6);(A4,I2,M6,E3,P7);(A4,I2,M6,E3,P8);(A4,I2,M6,E3,P9);(A4,I2,M6,E3,P10);(A4,I2,M6,E3,P11);(A4,I2,M6,E3,P12);(A4,I2,M6,E3,P13);(A4,I2,M6,E3,P14);(A4,I2,M6,E3,P15);(A4,I2,M6,E3,P16);(A4,I2,M6,E3,P17);(A4,I2,M6,E3,P18);(A4,I2,M6,E3,P19);(A4,I2,M6,E3,P20);(A4,I2,M6,E4,P1);(A4,I2,M6,E4,P2);(A4,I2,M6,E4,P3);(A4,I2,M6,E4,P4);(A4,I2,M6,E4,P5);(A4,I2,M6,E4,P6);(A4,I2,M6,E4,P7);(A4,I2,M6,E4,P8);(A4,I2,M6,E4,P9);(A4,I2,M6,E4,P10);(A4,I2,M6,E4,P11);(A4,I2,M6,E4,P12);(A4,I2,M6,E4,P13);(A4,I2,M6,E4,P14);(A4,I2,M6,E4,P15);(A4,I2,M6,E4,P16);(A4,I2,M6,E4,P17);(A4,I2,M6,E4,P18);(A4,I2,M6,E4,P19);(A4,I2,M6,E4,P20);(A4,I2,M6,E5,P1);(A4,I2,M6,E5,P2);(A4,I2,M6,E5,P3);(A4,I2,M6,E5,P4);(A4,I2,M6,E5,P5);(A4,I2,M6,E5,P6);(A4,I2,M6,E5,P7);(A4,I2,M6,E5,P8);(A4,I2,M6,E5,P9);(A4,I2,M6,E5,P10);(A4,I2,M6,E5,P11);(A4,I2,M6,E5,P12);(A4,I2,M6,E5,P13);(A4,I2,M6,E5,P14);(A4,I2,M6,E5,P15);(A4,I2,M6,E5,P16);(A4,I2,M6,E5,P17);(A4,I2,M6,E5,P18);(A4,I2,M6,E5,P19);(A4,I2,M6,E5,P20);(A4,I2,M6,E6,P1);(A4,I2,M6,E6,P2);(A4,I2,M6,E6,P3);(A4,I2,M6,E6,P4);(A4,I2,M6,E6,P5);(A4,I2,M6,E6,P6);(A4,I2,M6,E6,P7);(A4,I2,M6,E6,P8);(A4,I2,M6,E6,P9);(A4,I2,M6,E6,P10);(A4,I2,M6,E6,P11);(A4,I2,M6,E6,P12);(A4,I2,M6,E6,P13);(A4,I2,M6,E6,P14);(A4,I2,M6,E6,P15);(A4,I2,M6,E6,P16);(A4,I2,M6,E6,P17);(A4,I2,M6,E6,P18);(A4,I2,M6,E6,P19);(A4,I2,M6,E6,P20);(A4,I2,M6,E7,P1);(A4,I2,M6,E7,P2);(A4,I2,M6,E7,P3);(A4,I2,M6,E7,P4);(A4,I2,M6,E7,P5);(A4,I2,M6,E7,P6);(A4,I2,M6,E7,P7);(A4,I2,M6,E7,P8);(A4,I2,M6,E7,P9);(A4,I2,M6,E7,P10);(A4,I2,M6,E7,P11);(A4,I2,M6,E7,P12);(A4,I2,M6,E7,P13);(A4,I2,M6,E7,P14);(A4,I2,M6,E7,P15);(A4,I2,M6,E7,P16);(A4,I2,M6,E7,P17);(A4,I2,M6,E7,P18);(A4,I2,M6,E7,P19);(A4,I2,M6,E7,P20);(A4,I2,M6,E8,P1);(A4,I2,M6,E8,P2);(A4,I2,M6,E8,P3);(A4,I2,M6,E8,P4);(A4,I2,M6,E8,P5);(A4,I2,M6,E8,P6);(A4,I2,M6,E8,P7);(A4,I2,M6,E8,P8);(A4,I2,M6,E8,P9);(A4,I2,M6,E8,P10);(A4,I2,M6,E8,P11);(A4,I2,M6,E8,P12);(A4,I2,M6,E8,P13);(A4,I2,M6,E8,P14);(A4,I2,M6,E8,P15);(A4,I2,M6,E8,P16);(A4,I2,M6,E8,P17);(A4,I2,M6,E8,P18);(A4,I2,M6,E8,P19);(A4,I2,M6,E8,P20);(A4,I2,M6,E9,P1);(A4,I2,M6,E9,P2);(A4,I2,M6,E9,P3);(A4,I2,M6,E9,P4);(A4,I2,M6,E9,P5);(A4,I2,M6,E9,P6);(A4,I2,M6,E9,P7);(A4,I2,M6,E9,P8);(A4,I2,M6,E9,P9);(A4,I2,M6,E9,P10);(A4,I2,M6,E9,P11);(A4,I2,M6,E9,P12);(A4,I2,M6,E9,P13);(A4,I2,M6,E9,P14);(A4,I2,M6,E9,P15);(A4,I2,M6,E9,P16);(A4,I2,M6,E9,P17);(A4,I2,M6,E9,P18);(A4,I2,M6,E9,P19);(A4,I2,M6,E9,P20);(A4,I3,M1,E1,P1);(A4,I3,M1,E1,P2);(A4,I3,M1,E1,P3);(A4,I3,M1,E1,P4);(A4,I3,M1,E1,P5);(A4,I3,M1,E1,P6);(A4,I3,M1,E1,P7);(A4,I3,M1,E1,P8);(A4,I3,M1,E1,P9);(A4,I3,M1,E1,P10);(A4,I3,M1,E1,P11);(A4,I3,M1,E1,P12);(A4,I3,M1,E1,P13);(A4,I3,M1,E1,P14);(A4,I3,M1,E1,P15);(A4,I3,M1,E1,P16);(A4,I3,M1,E1,P17);(A4,I3,M1,E1,P18);(A4,I3,M1,E1,P19);(A4,I3,M1,E1,P20);(A4,I3,M1,E2,P1);(A4,I3,M1,E2,P2);(A4,I3,M1,E2,P3);(A4,I3,M1,E2,P4);(A4,I3,M1,E2,P5);(A4,I3,M1,E2,P6);(A4,I3,M1,E2,P7);(A4,I3,M1,E2,P8);(A4,I3,M1,E2,P9);(A4,I3,M1,E2,P10);(A4,I3,M1,E2,P11);(A4,I3,M1,E2,P12);(A4,I3,M1,E2,P13);(A4,I3,M1,E2,P14);(A4,I3,M1,E2,P15);(A4,I3,M1,E2,P16);(A4,I3,M1,E2,P17);(A4,I3,M1,E2,P18);(A4,I3,M1,E2,P19);(A4,I3,M1,E2,P20);(A4,I3,M1,E3,P1);(A4,I3,M1,E3,P2);(A4,I3,M1,E3,P3);(A4,I3,M1,E3,P4);(A4,I3,M1,E3,P5);(A4,I3,M1,E3,P6);(A4,I3,M1,E3,P7);(A4,I3,M1,E3,P8);(A4,I3,M1,E3,P9);(A4,I3,M1,E3,P10);(A4,I3,M1,E3,P11);(A4,I3,M1,E3,P12);(A4,I3,M1,E3,P13);(A4,I3,M1,E3,P14);(A4,I3,M1,E3,P15);(A4,I3,M1,E3,P16);(A4,I3,M1,E3,P17);(A4,I3,M1,E3,P18);(A4,I3,M1,E3,P19);(A4,I3,M1,E3,P20);(A4,I3,M1,E4,P1);(A4,I3,M1,E4,P2);(A4,I3,M1,E4,P3);(A4,I3,M1,E4,P4);(A4,I3,M1,E4,P5);(A4,I3,M1,E4,P6);(A4,I3,M1,E4,P7);(A4,I3,M1,E4,P8);(A4,I3,M1,E4,P9);(A4,I3,M1,E4,P10);(A4,I3,M1,E4,P11);(A4,I3,M1,E4,P12);(A4,I3,M1,E4,P13);(A4,I3,M1,E4,P14);(A4,I3,M1,E4,P15);(A4,I3,M1,E4,P16);(A4,I3,M1,E4,P17);(A4,I3,M1,E4,P18);(A4,I3,M1,E4,P19);(A4,I3,M1,E4,P20);(A4,I3,M1,E5,P1);(A4,I3,M1,E5,P2);(A4,I3,M1,E5,P3);(A4,I3,M1,E5,P4);(A4,I3,M1,E5,P5);(A4,I3,M1,E5,P6);(A4,I3,M1,E5,P7);(A4,I3,M1,E5,P8);(A4,I3,M1,E5,P9);(A4,I3,M1,E5,P10);(A4,I3,M1,E5,P11);(A4,I3,M1,E5,P12);(A4,I3,M1,E5,P13);(A4,I3,M1,E5,P14);(A4,I3,M1,E5,P15);(A4,I3,M1,E5,P16);(A4,I3,M1,E5,P17);(A4,I3,M1,E5,P18);(A4,I3,M1,E5,P19);(A4,I3,M1,E5,P20);(A4,I3,M1,E6,P1);(A4,I3,M1,E6,P2);(A4,I3,M1,E6,P3);(A4,I3,M1,E6,P4);(A4,I3,M1,E6,P5);(A4,I3,M1,E6,P6);(A4,I3,M1,E6,P7);(A4,I3,M1,E6,P8);(A4,I3,M1,E6,P9);(A4,I3,M1,E6,P10);(A4,I3,M1,E6,P11);(A4,I3,M1,E6,P12);(A4,I3,M1,E6,P13);(A4,I3,M1,E6,P14);(A4,I3,M1,E6,P15);(A4,I3,M1,E6,P16);(A4,I3,M1,E6,P17);(A4,I3,M1,E6,P18);(A4,I3,M1,E6,P19);(A4,I3,M1,E6,P20);(A4,I3,M1,E7,P1);(A4,I3,M1,E7,P2);(A4,I3,M1,E7,P3);(A4,I3,M1,E7,P4);(A4,I3,M1,E7,P5);(A4,I3,M1,E7,P6);(A4,I3,M1,E7,P7);(A4,I3,M1,E7,P8);(A4,I3,M1,E7,P9);(A4,I3,M1,E7,P10);(A4,I3,M1,E7,P11);(A4,I3,M1,E7,P12);(A4,I3,M1,E7,P13);(A4,I3,M1,E7,P14);(A4,I3,M1,E7,P15);(A4,I3,M1,E7,P16);(A4,I3,M1,E7,P17);(A4,I3,M1,E7,P18);(A4,I3,M1,E7,P19);(A4,I3,M1,E7,P20);(A4,I3,M1,E8,P1);(A4,I3,M1,E8,P2);(A4,I3,M1,E8,P3);(A4,I3,M1,E8,P4);(A4,I3,M1,E8,P5);(A4,I3,M1,E8,P6);(A4,I3,M1,E8,P7);(A4,I3,M1,E8,P8);(A4,I3,M1,E8,P9);(A4,I3,M1,E8,P10);(A4,I3,M1,E8,P11);(A4,I3,M1,E8,P12);(A4,I3,M1,E8,P13);(A4,I3,M1,E8,P14);(A4,I3,M1,E8,P15);(A4,I3,M1,E8,P16);(A4,I3,M1,E8,P17);(A4,I3,M1,E8,P18);(A4,I3,M1,E8,P19);(A4,I3,M1,E8,P20);(A4,I3,M1,E9,P1);(A4,I3,M1,E9,P2);(A4,I3,M1,E9,P3);(A4,I3,M1,E9,P4);(A4,I3,M1,E9,P5);(A4,I3,M1,E9,P6);(A4,I3,M1,E9,P7);(A4,I3,M1,E9,P8);(A4,I3,M1,E9,P9);(A4,I3,M1,E9,P10);(A4,I3,M1,E9,P11);(A4,I3,M1,E9,P12);(A4,I3,M1,E9,P13);(A4,I3,M1,E9,P14);(A4,I3,M1,E9,P15);(A4,I3,M1,E9,P16);(A4,I3,M1,E9,P17);(A4,I3,M1,E9,P18);(A4,I3,M1,E9,P19);(A4,I3,M1,E9,P20);(A4,I3,M2,E1,P1);(A4,I3,M2,E1,P2);(A4,I3,M2,E1,P3);(A4,I3,M2,E1,P4);(A4,I3,M2,E1,P5);(A4,I3,M2,E1,P6);(A4,I3,M2,E1,P7);(A4,I3,M2,E1,P8);(A4,I3,M2,E1,P9);(A4,I3,M2,E1,P10);(A4,I3,M2,E1,P11);(A4,I3,M2,E1,P12);(A4,I3,M2,E1,P13);(A4,I3,M2,E1,P14);(A4,I3,M2,E1,P15);(A4,I3,M2,E1,P16);(A4,I3,M2,E1,P17);(A4,I3,M2,E1,P18);(A4,I3,M2,E1,P19);(A4,I3,M2,E1,P20);(A4,I3,M2,E2,P1);(A4,I3,M2,E2,P2);(A4,I3,M2,E2,P3);(A4,I3,M2,E2,P4);(A4,I3,M2,E2,P5);(A4,I3,M2,E2,P6);(A4,I3,M2,E2,P7);(A4,I3,M2,E2,P8);(A4,I3,M2,E2,P9);(A4,I3,M2,E2,P10);(A4,I3,M2,E2,P11);(A4,I3,M2,E2,P12);(A4,I3,M2,E2,P13);(A4,I3,M2,E2,P14);(A4,I3,M2,E2,P15);(A4,I3,M2,E2,P16);(A4,I3,M2,E2,P17);(A4,I3,M2,E2,P18);(A4,I3,M2,E2,P19);(A4,I3,M2,E2,P20);(A4,I3,M2,E3,P1);(A4,I3,M2,E3,P2);(A4,I3,M2,E3,P3);(A4,I3,M2,E3,P4);(A4,I3,M2,E3,P5);(A4,I3,M2,E3,P6);(A4,I3,M2,E3,P7);(A4,I3,M2,E3,P8);(A4,I3,M2,E3,P9);(A4,I3,M2,E3,P10);(A4,I3,M2,E3,P11);(A4,I3,M2,E3,P12);(A4,I3,M2,E3,P13);(A4,I3,M2,E3,P14);(A4,I3,M2,E3,P15);(A4,I3,M2,E3,P16);(A4,I3,M2,E3,P17);(A4,I3,M2,E3,P18);(A4,I3,M2,E3,P19);(A4,I3,M2,E3,P20);(A4,I3,M2,E4,P1);(A4,I3,M2,E4,P2);(A4,I3,M2,E4,P3);(A4,I3,M2,E4,P4);(A4,I3,M2,E4,P5);(A4,I3,M2,E4,P6);(A4,I3,M2,E4,P7);(A4,I3,M2,E4,P8);(A4,I3,M2,E4,P9);(A4,I3,M2,E4,P10);(A4,I3,M2,E4,P11);(A4,I3,M2,E4,P12);(A4,I3,M2,E4,P13);(A4,I3,M2,E4,P14);(A4,I3,M2,E4,P15);(A4,I3,M2,E4,P16);(A4,I3,M2,E4,P17);(A4,I3,M2,E4,P18);(A4,I3,M2,E4,P19);(A4,I3,M2,E4,P20);(A4,I3,M2,E5,P1);(A4,I3,M2,E5,P2);(A4,I3,M2,E5,P3);(A4,I3,M2,E5,P4);(A4,I3,M2,E5,P5);(A4,I3,M2,E5,P6);(A4,I3,M2,E5,P7);(A4,I3,M2,E5,P8);(A4,I3,M2,E5,P9);(A4,I3,M2,E5,P10);(A4,I3,M2,E5,P11);(A4,I3,M2,E5,P12);(A4,I3,M2,E5,P13);(A4,I3,M2,E5,P14);(A4,I3,M2,E5,P15);(A4,I3,M2,E5,P16);(A4,I3,M2,E5,P17);(A4,I3,M2,E5,P18);(A4,I3,M2,E5,P19);(A4,I3,M2,E5,P20);(A4,I3,M2,E6,P1);(A4,I3,M2,E6,P2);(A4,I3,M2,E6,P3);(A4,I3,M2,E6,P4);(A4,I3,M2,E6,P5);(A4,I3,M2,E6,P6);(A4,I3,M2,E6,P7);(A4,I3,M2,E6,P8);(A4,I3,M2,E6,P9);(A4,I3,M2,E6,P10);(A4,I3,M2,E6,P11);(A4,I3,M2,E6,P12);(A4,I3,M2,E6,P13);(A4,I3,M2,E6,P14);(A4,I3,M2,E6,P15);(A4,I3,M2,E6,P16);(A4,I3,M2,E6,P17);(A4,I3,M2,E6,P18);(A4,I3,M2,E6,P19);(A4,I3,M2,E6,P20);(A4,I3,M2,E7,P1);(A4,I3,M2,E7,P2);(A4,I3,M2,E7,P3);(A4,I3,M2,E7,P4);(A4,I3,M2,E7,P5);(A4,I3,M2,E7,P6);(A4,I3,M2,E7,P7);(A4,I3,M2,E7,P8);(A4,I3,M2,E7,P9);(A4,I3,M2,E7,P10);(A4,I3,M2,E7,P11);(A4,I3,M2,E7,P12);(A4,I3,M2,E7,P13);(A4,I3,M2,E7,P14);(A4,I3,M2,E7,P15);(A4,I3,M2,E7,P16);(A4,I3,M2,E7,P17);(A4,I3,M2,E7,P18);(A4,I3,M2,E7,P19);(A4,I3,M2,E7,P20);(A4,I3,M2,E8,P1);(A4,I3,M2,E8,P2);(A4,I3,M2,E8,P3);(A4,I3,M2,E8,P4);(A4,I3,M2,E8,P5);(A4,I3,M2,E8,P6);(A4,I3,M2,E8,P7);(A4,I3,M2,E8,P8);(A4,I3,M2,E8,P9);(A4,I3,M2,E8,P10);(A4,I3,M2,E8,P11);(A4,I3,M2,E8,P12);(A4,I3,M2,E8,P13);(A4,I3,M2,E8,P14);(A4,I3,M2,E8,P15);(A4,I3,M2,E8,P16);(A4,I3,M2,E8,P17);(A4,I3,M2,E8,P18);(A4,I3,M2,E8,P19);(A4,I3,M2,E8,P20);(A4,I3,M2,E9,P1);(A4,I3,M2,E9,P2);(A4,I3,M2,E9,P3);(A4,I3,M2,E9,P4);(A4,I3,M2,E9,P5);(A4,I3,M2,E9,P6);(A4,I3,M2,E9,P7);(A4,I3,M2,E9,P8);(A4,I3,M2,E9,P9);(A4,I3,M2,E9,P10);(A4,I3,M2,E9,P11);(A4,I3,M2,E9,P12);(A4,I3,M2,E9,P13);(A4,I3,M2,E9,P14);(A4,I3,M2,E9,P15);(A4,I3,M2,E9,P16);(A4,I3,M2,E9,P17);(A4,I3,M2,E9,P18);(A4,I3,M2,E9,P19);(A4,I3,M2,E9,P20);(A4,I3,M3,E1,P1);(A4,I3,M3,E1,P2);(A4,I3,M3,E1,P3);(A4,I3,M3,E1,P4);(A4,I3,M3,E1,P5);(A4,I3,M3,E1,P6);(A4,I3,M3,E1,P7);(A4,I3,M3,E1,P8);(A4,I3,M3,E1,P9);(A4,I3,M3,E1,P10);(A4,I3,M3,E1,P11);(A4,I3,M3,E1,P12);(A4,I3,M3,E1,P13);(A4,I3,M3,E1,P14);(A4,I3,M3,E1,P15);(A4,I3,M3,E1,P16);(A4,I3,M3,E1,P17);(A4,I3,M3,E1,P18);(A4,I3,M3,E1,P19);(A4,I3,M3,E1,P20);(A4,I3,M3,E2,P1);(A4,I3,M3,E2,P2);(A4,I3,M3,E2,P3);(A4,I3,M3,E2,P4);(A4,I3,M3,E2,P5);(A4,I3,M3,E2,P6);(A4,I3,M3,E2,P7);(A4,I3,M3,E2,P8);(A4,I3,M3,E2,P9);(A4,I3,M3,E2,P10);(A4,I3,M3,E2,P11);(A4,I3,M3,E2,P12);(A4,I3,M3,E2,P13);(A4,I3,M3,E2,P14);(A4,I3,M3,E2,P15);(A4,I3,M3,E2,P16);(A4,I3,M3,E2,P17);(A4,I3,M3,E2,P18);(A4,I3,M3,E2,P19);(A4,I3,M3,E2,P20);(A4,I3,M3,E3,P1);(A4,I3,M3,E3,P2);(A4,I3,M3,E3,P3);(A4,I3,M3,E3,P4);(A4,I3,M3,E3,P5);(A4,I3,M3,E3,P6);(A4,I3,M3,E3,P7);(A4,I3,M3,E3,P8);(A4,I3,M3,E3,P9);(A4,I3,M3,E3,P10);(A4,I3,M3,E3,P11);(A4,I3,M3,E3,P12);(A4,I3,M3,E3,P13);(A4,I3,M3,E3,P14);(A4,I3,M3,E3,P15);(A4,I3,M3,E3,P16);(A4,I3,M3,E3,P17);(A4,I3,M3,E3,P18);(A4,I3,M3,E3,P19);(A4,I3,M3,E3,P20);(A4,I3,M3,E4,P1);(A4,I3,M3,E4,P2);(A4,I3,M3,E4,P3);(A4,I3,M3,E4,P4);(A4,I3,M3,E4,P5);(A4,I3,M3,E4,P6);(A4,I3,M3,E4,P7);(A4,I3,M3,E4,P8);(A4,I3,M3,E4,P9);(A4,I3,M3,E4,P10);(A4,I3,M3,E4,P11);(A4,I3,M3,E4,P12);(A4,I3,M3,E4,P13);(A4,I3,M3,E4,P14);(A4,I3,M3,E4,P15);(A4,I3,M3,E4,P16);(A4,I3,M3,E4,P17);(A4,I3,M3,E4,P18);(A4,I3,M3,E4,P19);(A4,I3,M3,E4,P20);(A4,I3,M3,E5,P1);(A4,I3,M3,E5,P2);(A4,I3,M3,E5,P3);(A4,I3,M3,E5,P4);(A4,I3,M3,E5,P5);(A4,I3,M3,E5,P6);(A4,I3,M3,E5,P7);(A4,I3,M3,E5,P8);(A4,I3,M3,E5,P9);(A4,I3,M3,E5,P10);(A4,I3,M3,E5,P11);(A4,I3,M3,E5,P12);(A4,I3,M3,E5,P13);(A4,I3,M3,E5,P14);(A4,I3,M3,E5,P15);(A4,I3,M3,E5,P16);(A4,I3,M3,E5,P17);(A4,I3,M3,E5,P18);(A4,I3,M3,E5,P19);(A4,I3,M3,E5,P20);(A4,I3,M3,E6,P1);(A4,I3,M3,E6,P2);(A4,I3,M3,E6,P3);(A4,I3,M3,E6,P4);(A4,I3,M3,E6,P5);(A4,I3,M3,E6,P6);(A4,I3,M3,E6,P7);(A4,I3,M3,E6,P8);(A4,I3,M3,E6,P9);(A4,I3,M3,E6,P10);(A4,I3,M3,E6,P11);(A4,I3,M3,E6,P12);(A4,I3,M3,E6,P13);(A4,I3,M3,E6,P14);(A4,I3,M3,E6,P15);(A4,I3,M3,E6,P16);(A4,I3,M3,E6,P17);(A4,I3,M3,E6,P18);(A4,I3,M3,E6,P19);(A4,I3,M3,E6,P20);(A4,I3,M3,E7,P1);(A4,I3,M3,E7,P2);(A4,I3,M3,E7,P3);(A4,I3,M3,E7,P4);(A4,I3,M3,E7,P5);(A4,I3,M3,E7,P6);(A4,I3,M3,E7,P7);(A4,I3,M3,E7,P8);(A4,I3,M3,E7,P9);(A4,I3,M3,E7,P10);(A4,I3,M3,E7,P11);(A4,I3,M3,E7,P12);(A4,I3,M3,E7,P13);(A4,I3,M3,E7,P14);(A4,I3,M3,E7,P15);(A4,I3,M3,E7,P16);(A4,I3,M3,E7,P17);(A4,I3,M3,E7,P18);(A4,I3,M3,E7,P19);(A4,I3,M3,E7,P20);(A4,I3,M3,E8,P1);(A4,I3,M3,E8,P2);(A4,I3,M3,E8,P3);(A4,I3,M3,E8,P4);(A4,I3,M3,E8,P5);(A4,I3,M3,E8,P6);(A4,I3,M3,E8,P7);(A4,I3,M3,E8,P8);(A4,I3,M3,E8,P9);(A4,I3,M3,E8,P10);(A4,I3,M3,E8,P11);(A4,I3,M3,E8,P12);(A4,I3,M3,E8,P13);(A4,I3,M3,E8,P14);(A4,I3,M3,E8,P15);(A4,I3,M3,E8,P16);(A4,I3,M3,E8,P17);(A4,I3,M3,E8,P18);(A4,I3,M3,E8,P19);(A4,I3,M3,E8,P20);(A4,I3,M3,E9,P1);(A4,I3,M3,E9,P2);(A4,I3,M3,E9,P3);(A4,I3,M3,E9,P4);(A4,I3,M3,E9,P5);(A4,I3,M3,E9,P6);(A4,I3,M3,E9,P7);(A4,I3,M3,E9,P8);(A4,I3,M3,E9,P9);(A4,I3,M3,E9,P10);(A4,I3,M3,E9,P11);(A4,I3,M3,E9,P12);(A4,I3,M3,E9,P13);(A4,I3,M3,E9,P14);(A4,I3,M3,E9,P15);(A4,I3,M3,E9,P16);(A4,I3,M3,E9,P17);(A4,I3,M3,E9,P18);(A4,I3,M3,E9,P19);(A4,I3,M3,E9,P20);(A4,I3,M4,E1,P1);(A4,I3,M4,E1,P2);(A4,I3,M4,E1,P3);(A4,I3,M4,E1,P4);(A4,I3,M4,E1,P5);(A4,I3,M4,E1,P6);(A4,I3,M4,E1,P7);(A4,I3,M4,E1,P8);(A4,I3,M4,E1,P9);(A4,I3,M4,E1,P10);(A4,I3,M4,E1,P11);(A4,I3,M4,E1,P12);(A4,I3,M4,E1,P13);(A4,I3,M4,E1,P14);(A4,I3,M4,E1,P15);(A4,I3,M4,E1,P16);(A4,I3,M4,E1,P17);(A4,I3,M4,E1,P18);(A4,I3,M4,E1,P19);(A4,I3,M4,E1,P20);(A4,I3,M4,E2,P1);(A4,I3,M4,E2,P2);(A4,I3,M4,E2,P3);(A4,I3,M4,E2,P4);(A4,I3,M4,E2,P5);(A4,I3,M4,E2,P6);(A4,I3,M4,E2,P7);(A4,I3,M4,E2,P8);(A4,I3,M4,E2,P9);(A4,I3,M4,E2,P10);(A4,I3,M4,E2,P11);(A4,I3,M4,E2,P12);(A4,I3,M4,E2,P13);(A4,I3,M4,E2,P14);(A4,I3,M4,E2,P15);(A4,I3,M4,E2,P16);(A4,I3,M4,E2,P17);(A4,I3,M4,E2,P18);(A4,I3,M4,E2,P19);(A4,I3,M4,E2,P20);(A4,I3,M4,E3,P1);(A4,I3,M4,E3,P2);(A4,I3,M4,E3,P3);(A4,I3,M4,E3,P4);(A4,I3,M4,E3,P5);(A4,I3,M4,E3,P6);(A4,I3,M4,E3,P7);(A4,I3,M4,E3,P8);(A4,I3,M4,E3,P9);(A4,I3,M4,E3,P10);(A4,I3,M4,E3,P11);(A4,I3,M4,E3,P12);(A4,I3,M4,E3,P13);(A4,I3,M4,E3,P14);(A4,I3,M4,E3,P15);(A4,I3,M4,E3,P16);(A4,I3,M4,E3,P17);(A4,I3,M4,E3,P18);(A4,I3,M4,E3,P19);(A4,I3,M4,E3,P20);(A4,I3,M4,E4,P1);(A4,I3,M4,E4,P2);(A4,I3,M4,E4,P3);(A4,I3,M4,E4,P4);(A4,I3,M4,E4,P5);(A4,I3,M4,E4,P6);(A4,I3,M4,E4,P7);(A4,I3,M4,E4,P8);(A4,I3,M4,E4,P9);(A4,I3,M4,E4,P10);(A4,I3,M4,E4,P11);(A4,I3,M4,E4,P12);(A4,I3,M4,E4,P13);(A4,I3,M4,E4,P14);(A4,I3,M4,E4,P15);(A4,I3,M4,E4,P16);(A4,I3,M4,E4,P17);(A4,I3,M4,E4,P18);(A4,I3,M4,E4,P19);(A4,I3,M4,E4,P20);(A4,I3,M4,E5,P1);(A4,I3,M4,E5,P2);(A4,I3,M4,E5,P3);(A4,I3,M4,E5,P4);(A4,I3,M4,E5,P5);(A4,I3,M4,E5,P6);(A4,I3,M4,E5,P7);(A4,I3,M4,E5,P8);(A4,I3,M4,E5,P9);(A4,I3,M4,E5,P10);(A4,I3,M4,E5,P11);(A4,I3,M4,E5,P12);(A4,I3,M4,E5,P13);(A4,I3,M4,E5,P14);(A4,I3,M4,E5,P15);(A4,I3,M4,E5,P16);(A4,I3,M4,E5,P17);(A4,I3,M4,E5,P18);(A4,I3,M4,E5,P19);(A4,I3,M4,E5,P20);(A4,I3,M4,E6,P1);(A4,I3,M4,E6,P2);(A4,I3,M4,E6,P3);(A4,I3,M4,E6,P4);(A4,I3,M4,E6,P5);(A4,I3,M4,E6,P6);(A4,I3,M4,E6,P7);(A4,I3,M4,E6,P8);(A4,I3,M4,E6,P9);(A4,I3,M4,E6,P10);(A4,I3,M4,E6,P11);(A4,I3,M4,E6,P12);(A4,I3,M4,E6,P13);(A4,I3,M4,E6,P14);(A4,I3,M4,E6,P15);(A4,I3,M4,E6,P16);(A4,I3,M4,E6,P17);(A4,I3,M4,E6,P18);(A4,I3,M4,E6,P19);(A4,I3,M4,E6,P20);(A4,I3,M4,E7,P1);(A4,I3,M4,E7,P2);(A4,I3,M4,E7,P3);(A4,I3,M4,E7,P4);(A4,I3,M4,E7,P5);(A4,I3,M4,E7,P6);(A4,I3,M4,E7,P7);(A4,I3,M4,E7,P8);(A4,I3,M4,E7,P9);(A4,I3,M4,E7,P10);(A4,I3,M4,E7,P11);(A4,I3,M4,E7,P12);(A4,I3,M4,E7,P13);(A4,I3,M4,E7,P14);(A4,I3,M4,E7,P15);(A4,I3,M4,E7,P16);(A4,I3,M4,E7,P17);(A4,I3,M4,E7,P18);(A4,I3,M4,E7,P19);(A4,I3,M4,E7,P20);(A4,I3,M4,E8,P1);(A4,I3,M4,E8,P2);(A4,I3,M4,E8,P3);(A4,I3,M4,E8,P4);(A4,I3,M4,E8,P5);(A4,I3,M4,E8,P6);(A4,I3,M4,E8,P7);(A4,I3,M4,E8,P8);(A4,I3,M4,E8,P9);(A4,I3,M4,E8,P10);(A4,I3,M4,E8,P11);(A4,I3,M4,E8,P12);(A4,I3,M4,E8,P13);(A4,I3,M4,E8,P14);(A4,I3,M4,E8,P15);(A4,I3,M4,E8,P16);(A4,I3,M4,E8,P17);(A4,I3,M4,E8,P18);(A4,I3,M4,E8,P19);(A4,I3,M4,E8,P20);(A4,I3,M4,E9,P1);(A4,I3,M4,E9,P2);(A4,I3,M4,E9,P3);(A4,I3,M4,E9,P4);(A4,I3,M4,E9,P5);(A4,I3,M4,E9,P6);(A4,I3,M4,E9,P7);(A4,I3,M4,E9,P8);(A4,I3,M4,E9,P9);(A4,I3,M4,E9,P10);(A4,I3,M4,E9,P11);(A4,I3,M4,E9,P12);(A4,I3,M4,E9,P13);(A4,I3,M4,E9,P14);(A4,I3,M4,E9,P15);(A4,I3,M4,E9,P16);(A4,I3,M4,E9,P17);(A4,I3,M4,E9,P18);(A4,I3,M4,E9,P19);(A4,I3,M4,E9,P20);(A4,I3,M5,E1,P1);(A4,I3,M5,E1,P2);(A4,I3,M5,E1,P3);(A4,I3,M5,E1,P4);(A4,I3,M5,E1,P5);(A4,I3,M5,E1,P6);(A4,I3,M5,E1,P7);(A4,I3,M5,E1,P8);(A4,I3,M5,E1,P9);(A4,I3,M5,E1,P10);(A4,I3,M5,E1,P11);(A4,I3,M5,E1,P12);(A4,I3,M5,E1,P13);(A4,I3,M5,E1,P14);(A4,I3,M5,E1,P15);(A4,I3,M5,E1,P16);(A4,I3,M5,E1,P17);(A4,I3,M5,E1,P18);(A4,I3,M5,E1,P19);(A4,I3,M5,E1,P20);(A4,I3,M5,E2,P1);(A4,I3,M5,E2,P2);(A4,I3,M5,E2,P3);(A4,I3,M5,E2,P4);(A4,I3,M5,E2,P5);(A4,I3,M5,E2,P6);(A4,I3,M5,E2,P7);(A4,I3,M5,E2,P8);(A4,I3,M5,E2,P9);(A4,I3,M5,E2,P10);(A4,I3,M5,E2,P11);(A4,I3,M5,E2,P12);(A4,I3,M5,E2,P13);(A4,I3,M5,E2,P14);(A4,I3,M5,E2,P15);(A4,I3,M5,E2,P16);(A4,I3,M5,E2,P17);(A4,I3,M5,E2,P18);(A4,I3,M5,E2,P19);(A4,I3,M5,E2,P20);(A4,I3,M5,E3,P1);(A4,I3,M5,E3,P2);(A4,I3,M5,E3,P3);(A4,I3,M5,E3,P4);(A4,I3,M5,E3,P5);(A4,I3,M5,E3,P6);(A4,I3,M5,E3,P7);(A4,I3,M5,E3,P8);(A4,I3,M5,E3,P9);(A4,I3,M5,E3,P10);(A4,I3,M5,E3,P11);(A4,I3,M5,E3,P12);(A4,I3,M5,E3,P13);(A4,I3,M5,E3,P14);(A4,I3,M5,E3,P15);(A4,I3,M5,E3,P16);(A4,I3,M5,E3,P17);(A4,I3,M5,E3,P18);(A4,I3,M5,E3,P19);(A4,I3,M5,E3,P20);(A4,I3,M5,E4,P1);(A4,I3,M5,E4,P2);(A4,I3,M5,E4,P3);(A4,I3,M5,E4,P4);(A4,I3,M5,E4,P5);(A4,I3,M5,E4,P6);(A4,I3,M5,E4,P7);(A4,I3,M5,E4,P8);(A4,I3,M5,E4,P9);(A4,I3,M5,E4,P10);(A4,I3,M5,E4,P11);(A4,I3,M5,E4,P12);(A4,I3,M5,E4,P13);(A4,I3,M5,E4,P14);(A4,I3,M5,E4,P15);(A4,I3,M5,E4,P16);(A4,I3,M5,E4,P17);(A4,I3,M5,E4,P18);(A4,I3,M5,E4,P19);(A4,I3,M5,E4,P20);(A4,I3,M5,E5,P1);(A4,I3,M5,E5,P2);(A4,I3,M5,E5,P3);(A4,I3,M5,E5,P4);(A4,I3,M5,E5,P5);(A4,I3,M5,E5,P6);(A4,I3,M5,E5,P7);(A4,I3,M5,E5,P8);(A4,I3,M5,E5,P9);(A4,I3,M5,E5,P10);(A4,I3,M5,E5,P11);(A4,I3,M5,E5,P12);(A4,I3,M5,E5,P13);(A4,I3,M5,E5,P14);(A4,I3,M5,E5,P15);(A4,I3,M5,E5,P16);(A4,I3,M5,E5,P17);(A4,I3,M5,E5,P18);(A4,I3,M5,E5,P19);(A4,I3,M5,E5,P20);(A4,I3,M5,E6,P1);(A4,I3,M5,E6,P2);(A4,I3,M5,E6,P3);(A4,I3,M5,E6,P4);(A4,I3,M5,E6,P5);(A4,I3,M5,E6,P6);(A4,I3,M5,E6,P7);(A4,I3,M5,E6,P8);(A4,I3,M5,E6,P9);(A4,I3,M5,E6,P10);(A4,I3,M5,E6,P11);(A4,I3,M5,E6,P12);(A4,I3,M5,E6,P13);(A4,I3,M5,E6,P14);(A4,I3,M5,E6,P15);(A4,I3,M5,E6,P16);(A4,I3,M5,E6,P17);(A4,I3,M5,E6,P18);(A4,I3,M5,E6,P19);(A4,I3,M5,E6,P20);(A4,I3,M5,E7,P1);(A4,I3,M5,E7,P2);(A4,I3,M5,E7,P3);(A4,I3,M5,E7,P4);(A4,I3,M5,E7,P5);(A4,I3,M5,E7,P6);(A4,I3,M5,E7,P7);(A4,I3,M5,E7,P8);(A4,I3,M5,E7,P9);(A4,I3,M5,E7,P10);(A4,I3,M5,E7,P11);(A4,I3,M5,E7,P12);(A4,I3,M5,E7,P13);(A4,I3,M5,E7,P14);(A4,I3,M5,E7,P15);(A4,I3,M5,E7,P16);(A4,I3,M5,E7,P17);(A4,I3,M5,E7,P18);(A4,I3,M5,E7,P19);(A4,I3,M5,E7,P20);(A4,I3,M5,E8,P1);(A4,I3,M5,E8,P2);(A4,I3,M5,E8,P3);(A4,I3,M5,E8,P4);(A4,I3,M5,E8,P5);(A4,I3,M5,E8,P6);(A4,I3,M5,E8,P7);(A4,I3,M5,E8,P8);(A4,I3,M5,E8,P9);(A4,I3,M5,E8,P10);(A4,I3,M5,E8,P11);(A4,I3,M5,E8,P12);(A4,I3,M5,E8,P13);(A4,I3,M5,E8,P14);(A4,I3,M5,E8,P15);(A4,I3,M5,E8,P16);(A4,I3,M5,E8,P17);(A4,I3,M5,E8,P18);(A4,I3,M5,E8,P19);(A4,I3,M5,E8,P20);(A4,I3,M5,E9,P1);(A4,I3,M5,E9,P2);(A4,I3,M5,E9,P3);(A4,I3,M5,E9,P4);(A4,I3,M5,E9,P5);(A4,I3,M5,E9,P6);(A4,I3,M5,E9,P7);(A4,I3,M5,E9,P8);(A4,I3,M5,E9,P9);(A4,I3,M5,E9,P10);(A4,I3,M5,E9,P11);(A4,I3,M5,E9,P12);(A4,I3,M5,E9,P13);(A4,I3,M5,E9,P14);(A4,I3,M5,E9,P15);(A4,I3,M5,E9,P16);(A4,I3,M5,E9,P17);(A4,I3,M5,E9,P18);(A4,I3,M5,E9,P19);(A4,I3,M5,E9,P20);(A4,I3,M6,E1,P1);(A4,I3,M6,E1,P2);(A4,I3,M6,E1,P3);(A4,I3,M6,E1,P4);(A4,I3,M6,E1,P5);(A4,I3,M6,E1,P6);(A4,I3,M6,E1,P7);(A4,I3,M6,E1,P8);(A4,I3,M6,E1,P9);(A4,I3,M6,E1,P10);(A4,I3,M6,E1,P11);(A4,I3,M6,E1,P12);(A4,I3,M6,E1,P13);(A4,I3,M6,E1,P14);(A4,I3,M6,E1,P15);(A4,I3,M6,E1,P16);(A4,I3,M6,E1,P17);(A4,I3,M6,E1,P18);(A4,I3,M6,E1,P19);(A4,I3,M6,E1,P20);(A4,I3,M6,E2,P1);(A4,I3,M6,E2,P2);(A4,I3,M6,E2,P3);(A4,I3,M6,E2,P4);(A4,I3,M6,E2,P5);(A4,I3,M6,E2,P6);(A4,I3,M6,E2,P7);(A4,I3,M6,E2,P8);(A4,I3,M6,E2,P9);(A4,I3,M6,E2,P10);(A4,I3,M6,E2,P11);(A4,I3,M6,E2,P12);(A4,I3,M6,E2,P13);(A4,I3,M6,E2,P14);(A4,I3,M6,E2,P15);(A4,I3,M6,E2,P16);(A4,I3,M6,E2,P17);(A4,I3,M6,E2,P18);(A4,I3,M6,E2,P19);(A4,I3,M6,E2,P20);(A4,I3,M6,E3,P1);(A4,I3,M6,E3,P2);(A4,I3,M6,E3,P3);(A4,I3,M6,E3,P4);(A4,I3,M6,E3,P5);(A4,I3,M6,E3,P6);(A4,I3,M6,E3,P7);(A4,I3,M6,E3,P8);(A4,I3,M6,E3,P9);(A4,I3,M6,E3,P10);(A4,I3,M6,E3,P11);(A4,I3,M6,E3,P12);(A4,I3,M6,E3,P13);(A4,I3,M6,E3,P14);(A4,I3,M6,E3,P15);(A4,I3,M6,E3,P16);(A4,I3,M6,E3,P17);(A4,I3,M6,E3,P18);(A4,I3,M6,E3,P19);(A4,I3,M6,E3,P20);(A4,I3,M6,E4,P1);(A4,I3,M6,E4,P2);(A4,I3,M6,E4,P3);(A4,I3,M6,E4,P4);(A4,I3,M6,E4,P5);(A4,I3,M6,E4,P6);(A4,I3,M6,E4,P7);(A4,I3,M6,E4,P8);(A4,I3,M6,E4,P9);(A4,I3,M6,E4,P10);(A4,I3,M6,E4,P11);(A4,I3,M6,E4,P12);(A4,I3,M6,E4,P13);(A4,I3,M6,E4,P14);(A4,I3,M6,E4,P15);(A4,I3,M6,E4,P16);(A4,I3,M6,E4,P17);(A4,I3,M6,E4,P18);(A4,I3,M6,E4,P19);(A4,I3,M6,E4,P20);(A4,I3,M6,E5,P1);(A4,I3,M6,E5,P2);(A4,I3,M6,E5,P3);(A4,I3,M6,E5,P4);(A4,I3,M6,E5,P5);(A4,I3,M6,E5,P6);(A4,I3,M6,E5,P7);(A4,I3,M6,E5,P8);(A4,I3,M6,E5,P9);(A4,I3,M6,E5,P10);(A4,I3,M6,E5,P11);(A4,I3,M6,E5,P12);(A4,I3,M6,E5,P13);(A4,I3,M6,E5,P14);(A4,I3,M6,E5,P15);(A4,I3,M6,E5,P16);(A4,I3,M6,E5,P17);(A4,I3,M6,E5,P18);(A4,I3,M6,E5,P19);(A4,I3,M6,E5,P20);(A4,I3,M6,E6,P1);(A4,I3,M6,E6,P2);(A4,I3,M6,E6,P3);(A4,I3,M6,E6,P4);(A4,I3,M6,E6,P5);(A4,I3,M6,E6,P6);(A4,I3,M6,E6,P7);(A4,I3,M6,E6,P8);(A4,I3,M6,E6,P9);(A4,I3,M6,E6,P10);(A4,I3,M6,E6,P11);(A4,I3,M6,E6,P12);(A4,I3,M6,E6,P13);(A4,I3,M6,E6,P14);(A4,I3,M6,E6,P15);(A4,I3,M6,E6,P16);(A4,I3,M6,E6,P17);(A4,I3,M6,E6,P18);(A4,I3,M6,E6,P19);(A4,I3,M6,E6,P20);(A4,I3,M6,E7,P1);(A4,I3,M6,E7,P2);(A4,I3,M6,E7,P3);(A4,I3,M6,E7,P4);(A4,I3,M6,E7,P5);(A4,I3,M6,E7,P6);(A4,I3,M6,E7,P7);(A4,I3,M6,E7,P8);(A4,I3,M6,E7,P9);(A4,I3,M6,E7,P10);(A4,I3,M6,E7,P11);(A4,I3,M6,E7,P12);(A4,I3,M6,E7,P13);(A4,I3,M6,E7,P14);(A4,I3,M6,E7,P15);(A4,I3,M6,E7,P16);(A4,I3,M6,E7,P17);(A4,I3,M6,E7,P18);(A4,I3,M6,E7,P19);(A4,I3,M6,E7,P20);(A4,I3,M6,E8,P1);(A4,I3,M6,E8,P2);(A4,I3,M6,E8,P3);(A4,I3,M6,E8,P4);(A4,I3,M6,E8,P5);(A4,I3,M6,E8,P6);(A4,I3,M6,E8,P7);(A4,I3,M6,E8,P8);(A4,I3,M6,E8,P9);(A4,I3,M6,E8,P10);(A4,I3,M6,E8,P11);(A4,I3,M6,E8,P12);(A4,I3,M6,E8,P13);(A4,I3,M6,E8,P14);(A4,I3,M6,E8,P15);(A4,I3,M6,E8,P16);(A4,I3,M6,E8,P17);(A4,I3,M6,E8,P18);(A4,I3,M6,E8,P19);(A4,I3,M6,E8,P20);(A4,I3,M6,E9,P1);(A4,I3,M6,E9,P2);(A4,I3,M6,E9,P3);(A4,I3,M6,E9,P4);(A4,I3,M6,E9,P5);(A4,I3,M6,E9,P6);(A4,I3,M6,E9,P7);(A4,I3,M6,E9,P8);(A4,I3,M6,E9,P9);(A4,I3,M6,E9,P10);(A4,I3,M6,E9,P11);(A4,I3,M6,E9,P12);(A4,I3,M6,E9,P13);(A4,I3,M6,E9,P14);(A4,I3,M6,E9,P15);(A4,I3,M6,E9,P16);(A4,I3,M6,E9,P17);(A4,I3,M6,E9,P18);(A4,I3,M6,E9,P19);(A4,I3,M6,E9,P20);(A5,I1,M1,E1,P1);(A5,I1,M1,E1,P2);(A5,I1,M1,E1,P3);(A5,I1,M1,E1,P4);(A5,I1,M1,E1,P5);(A5,I1,M1,E1,P6);(A5,I1,M1,E1,P7);(A5,I1,M1,E1,P8);(A5,I1,M1,E1,P9);(A5,I1,M1,E1,P10);(A5,I1,M1,E1,P11);(A5,I1,M1,E1,P12);(A5,I1,M1,E1,P13);(A5,I1,M1,E1,P14);(A5,I1,M1,E1,P15);(A5,I1,M1,E1,P16);(A5,I1,M1,E1,P17);(A5,I1,M1,E1,P18);(A5,I1,M1,E1,P19);(A5,I1,M1,E1,P20);(A5,I1,M1,E2,P1);(A5,I1,M1,E2,P2);(A5,I1,M1,E2,P3);(A5,I1,M1,E2,P4);(A5,I1,M1,E2,P5);(A5,I1,M1,E2,P6);(A5,I1,M1,E2,P7);(A5,I1,M1,E2,P8);(A5,I1,M1,E2,P9);(A5,I1,M1,E2,P10);(A5,I1,M1,E2,P11);(A5,I1,M1,E2,P12);(A5,I1,M1,E2,P13);(A5,I1,M1,E2,P14);(A5,I1,M1,E2,P15);(A5,I1,M1,E2,P16);(A5,I1,M1,E2,P17);(A5,I1,M1,E2,P18);(A5,I1,M1,E2,P19);(A5,I1,M1,E2,P20);(A5,I1,M1,E3,P1);(A5,I1,M1,E3,P2);(A5,I1,M1,E3,P3);(A5,I1,M1,E3,P4);(A5,I1,M1,E3,P5);(A5,I1,M1,E3,P6);(A5,I1,M1,E3,P7);(A5,I1,M1,E3,P8);(A5,I1,M1,E3,P9);(A5,I1,M1,E3,P10);(A5,I1,M1,E3,P11);(A5,I1,M1,E3,P12);(A5,I1,M1,E3,P13);(A5,I1,M1,E3,P14);(A5,I1,M1,E3,P15);(A5,I1,M1,E3,P16);(A5,I1,M1,E3,P17);(A5,I1,M1,E3,P18);(A5,I1,M1,E3,P19);(A5,I1,M1,E3,P20);(A5,I1,M1,E4,P1);(A5,I1,M1,E4,P2);(A5,I1,M1,E4,P3);(A5,I1,M1,E4,P4);(A5,I1,M1,E4,P5);(A5,I1,M1,E4,P6);(A5,I1,M1,E4,P7);(A5,I1,M1,E4,P8);(A5,I1,M1,E4,P9);(A5,I1,M1,E4,P10);(A5,I1,M1,E4,P11);(A5,I1,M1,E4,P12);(A5,I1,M1,E4,P13);(A5,I1,M1,E4,P14);(A5,I1,M1,E4,P15);(A5,I1,M1,E4,P16);(A5,I1,M1,E4,P17);(A5,I1,M1,E4,P18);(A5,I1,M1,E4,P19);(A5,I1,M1,E4,P20);(A5,I1,M1,E5,P1);(A5,I1,M1,E5,P2);(A5,I1,M1,E5,P3);(A5,I1,M1,E5,P4);(A5,I1,M1,E5,P5);(A5,I1,M1,E5,P6);(A5,I1,M1,E5,P7);(A5,I1,M1,E5,P8);(A5,I1,M1,E5,P9);(A5,I1,M1,E5,P10);(A5,I1,M1,E5,P11);(A5,I1,M1,E5,P12);(A5,I1,M1,E5,P13);(A5,I1,M1,E5,P14);(A5,I1,M1,E5,P15);(A5,I1,M1,E5,P16);(A5,I1,M1,E5,P17);(A5,I1,M1,E5,P18);(A5,I1,M1,E5,P19);(A5,I1,M1,E5,P20);(A5,I1,M1,E6,P1);(A5,I1,M1,E6,P2);(A5,I1,M1,E6,P3);(A5,I1,M1,E6,P4);(A5,I1,M1,E6,P5);(A5,I1,M1,E6,P6);(A5,I1,M1,E6,P7);(A5,I1,M1,E6,P8);(A5,I1,M1,E6,P9);(A5,I1,M1,E6,P10);(A5,I1,M1,E6,P11);(A5,I1,M1,E6,P12);(A5,I1,M1,E6,P13);(A5,I1,M1,E6,P14);(A5,I1,M1,E6,P15);(A5,I1,M1,E6,P16);(A5,I1,M1,E6,P17);(A5,I1,M1,E6,P18);(A5,I1,M1,E6,P19);(A5,I1,M1,E6,P20);(A5,I1,M1,E7,P1);(A5,I1,M1,E7,P2);(A5,I1,M1,E7,P3);(A5,I1,M1,E7,P4);(A5,I1,M1,E7,P5);(A5,I1,M1,E7,P6);(A5,I1,M1,E7,P7);(A5,I1,M1,E7,P8);(A5,I1,M1,E7,P9);(A5,I1,M1,E7,P10);(A5,I1,M1,E7,P11);(A5,I1,M1,E7,P12);(A5,I1,M1,E7,P13);(A5,I1,M1,E7,P14);(A5,I1,M1,E7,P15);(A5,I1,M1,E7,P16);(A5,I1,M1,E7,P17);(A5,I1,M1,E7,P18);(A5,I1,M1,E7,P19);(A5,I1,M1,E7,P20);(A5,I1,M1,E8,P1);(A5,I1,M1,E8,P2);(A5,I1,M1,E8,P3);(A5,I1,M1,E8,P4);(A5,I1,M1,E8,P5);(A5,I1,M1,E8,P6);(A5,I1,M1,E8,P7);(A5,I1,M1,E8,P8);(A5,I1,M1,E8,P9);(A5,I1,M1,E8,P10);(A5,I1,M1,E8,P11);(A5,I1,M1,E8,P12);(A5,I1,M1,E8,P13);(A5,I1,M1,E8,P14);(A5,I1,M1,E8,P15);(A5,I1,M1,E8,P16);(A5,I1,M1,E8,P17);(A5,I1,M1,E8,P18);(A5,I1,M1,E8,P19);(A5,I1,M1,E8,P20);(A5,I1,M1,E9,P1);(A5,I1,M1,E9,P2);(A5,I1,M1,E9,P3);(A5,I1,M1,E9,P4);(A5,I1,M1,E9,P5);(A5,I1,M1,E9,P6);(A5,I1,M1,E9,P7);(A5,I1,M1,E9,P8);(A5,I1,M1,E9,P9);(A5,I1,M1,E9,P10);(A5,I1,M1,E9,P11);(A5,I1,M1,E9,P12);(A5,I1,M1,E9,P13);(A5,I1,M1,E9,P14);(A5,I1,M1,E9,P15);(A5,I1,M1,E9,P16);(A5,I1,M1,E9,P17);(A5,I1,M1,E9,P18);(A5,I1,M1,E9,P19);(A5,I1,M1,E9,P20);(A5,I1,M2,E1,P1);(A5,I1,M2,E1,P2);(A5,I1,M2,E1,P3);(A5,I1,M2,E1,P4);(A5,I1,M2,E1,P5);(A5,I1,M2,E1,P6);(A5,I1,M2,E1,P7);(A5,I1,M2,E1,P8);(A5,I1,M2,E1,P9);(A5,I1,M2,E1,P10);(A5,I1,M2,E1,P11);(A5,I1,M2,E1,P12);(A5,I1,M2,E1,P13);(A5,I1,M2,E1,P14);(A5,I1,M2,E1,P15);(A5,I1,M2,E1,P16);(A5,I1,M2,E1,P17);(A5,I1,M2,E1,P18);(A5,I1,M2,E1,P19);(A5,I1,M2,E1,P20);(A5,I1,M2,E2,P1);(A5,I1,M2,E2,P2);(A5,I1,M2,E2,P3);(A5,I1,M2,E2,P4);(A5,I1,M2,E2,P5);(A5,I1,M2,E2,P6);(A5,I1,M2,E2,P7);(A5,I1,M2,E2,P8);(A5,I1,M2,E2,P9);(A5,I1,M2,E2,P10);(A5,I1,M2,E2,P11);(A5,I1,M2,E2,P12);(A5,I1,M2,E2,P13);(A5,I1,M2,E2,P14);(A5,I1,M2,E2,P15);(A5,I1,M2,E2,P16);(A5,I1,M2,E2,P17);(A5,I1,M2,E2,P18);(A5,I1,M2,E2,P19);(A5,I1,M2,E2,P20);(A5,I1,M2,E3,P1);(A5,I1,M2,E3,P2);(A5,I1,M2,E3,P3);(A5,I1,M2,E3,P4);(A5,I1,M2,E3,P5);(A5,I1,M2,E3,P6);(A5,I1,M2,E3,P7);(A5,I1,M2,E3,P8);(A5,I1,M2,E3,P9);(A5,I1,M2,E3,P10);(A5,I1,M2,E3,P11);(A5,I1,M2,E3,P12);(A5,I1,M2,E3,P13);(A5,I1,M2,E3,P14);(A5,I1,M2,E3,P15);(A5,I1,M2,E3,P16);(A5,I1,M2,E3,P17);(A5,I1,M2,E3,P18);(A5,I1,M2,E3,P19);(A5,I1,M2,E3,P20);(A5,I1,M2,E4,P1);(A5,I1,M2,E4,P2);(A5,I1,M2,E4,P3);(A5,I1,M2,E4,P4);(A5,I1,M2,E4,P5);(A5,I1,M2,E4,P6);(A5,I1,M2,E4,P7);(A5,I1,M2,E4,P8);(A5,I1,M2,E4,P9);(A5,I1,M2,E4,P10);(A5,I1,M2,E4,P11);(A5,I1,M2,E4,P12);(A5,I1,M2,E4,P13);(A5,I1,M2,E4,P14);(A5,I1,M2,E4,P15);(A5,I1,M2,E4,P16);(A5,I1,M2,E4,P17);(A5,I1,M2,E4,P18);(A5,I1,M2,E4,P19);(A5,I1,M2,E4,P20);(A5,I1,M2,E5,P1);(A5,I1,M2,E5,P2);(A5,I1,M2,E5,P3);(A5,I1,M2,E5,P4);(A5,I1,M2,E5,P5);(A5,I1,M2,E5,P6);(A5,I1,M2,E5,P7);(A5,I1,M2,E5,P8);(A5,I1,M2,E5,P9);(A5,I1,M2,E5,P10);(A5,I1,M2,E5,P11);(A5,I1,M2,E5,P12);(A5,I1,M2,E5,P13);(A5,I1,M2,E5,P14);(A5,I1,M2,E5,P15);(A5,I1,M2,E5,P16);(A5,I1,M2,E5,P17);(A5,I1,M2,E5,P18);(A5,I1,M2,E5,P19);(A5,I1,M2,E5,P20);(A5,I1,M2,E6,P1);(A5,I1,M2,E6,P2);(A5,I1,M2,E6,P3);(A5,I1,M2,E6,P4);(A5,I1,M2,E6,P5);(A5,I1,M2,E6,P6);(A5,I1,M2,E6,P7);(A5,I1,M2,E6,P8);(A5,I1,M2,E6,P9);(A5,I1,M2,E6,P10);(A5,I1,M2,E6,P11);(A5,I1,M2,E6,P12);(A5,I1,M2,E6,P13);(A5,I1,M2,E6,P14);(A5,I1,M2,E6,P15);(A5,I1,M2,E6,P16);(A5,I1,M2,E6,P17);(A5,I1,M2,E6,P18);(A5,I1,M2,E6,P19);(A5,I1,M2,E6,P20);(A5,I1,M2,E7,P1);(A5,I1,M2,E7,P2);(A5,I1,M2,E7,P3);(A5,I1,M2,E7,P4);(A5,I1,M2,E7,P5);(A5,I1,M2,E7,P6);(A5,I1,M2,E7,P7);(A5,I1,M2,E7,P8);(A5,I1,M2,E7,P9);(A5,I1,M2,E7,P10);(A5,I1,M2,E7,P11);(A5,I1,M2,E7,P12);(A5,I1,M2,E7,P13);(A5,I1,M2,E7,P14);(A5,I1,M2,E7,P15);(A5,I1,M2,E7,P16);(A5,I1,M2,E7,P17);(A5,I1,M2,E7,P18);(A5,I1,M2,E7,P19);(A5,I1,M2,E7,P20);(A5,I1,M2,E8,P1);(A5,I1,M2,E8,P2);(A5,I1,M2,E8,P3);(A5,I1,M2,E8,P4);(A5,I1,M2,E8,P5);(A5,I1,M2,E8,P6);(A5,I1,M2,E8,P7);(A5,I1,M2,E8,P8);(A5,I1,M2,E8,P9);(A5,I1,M2,E8,P10);(A5,I1,M2,E8,P11);(A5,I1,M2,E8,P12);(A5,I1,M2,E8,P13);(A5,I1,M2,E8,P14);(A5,I1,M2,E8,P15);(A5,I1,M2,E8,P16);(A5,I1,M2,E8,P17);(A5,I1,M2,E8,P18);(A5,I1,M2,E8,P19);(A5,I1,M2,E8,P20);(A5,I1,M2,E9,P1);(A5,I1,M2,E9,P2);(A5,I1,M2,E9,P3);(A5,I1,M2,E9,P4);(A5,I1,M2,E9,P5);(A5,I1,M2,E9,P6);(A5,I1,M2,E9,P7);(A5,I1,M2,E9,P8);(A5,I1,M2,E9,P9);(A5,I1,M2,E9,P10);(A5,I1,M2,E9,P11);(A5,I1,M2,E9,P12);(A5,I1,M2,E9,P13);(A5,I1,M2,E9,P14);(A5,I1,M2,E9,P15);(A5,I1,M2,E9,P16);(A5,I1,M2,E9,P17);(A5,I1,M2,E9,P18);(A5,I1,M2,E9,P19);(A5,I1,M2,E9,P20);(A5,I1,M3,E1,P1);(A5,I1,M3,E1,P2);(A5,I1,M3,E1,P3);(A5,I1,M3,E1,P4);(A5,I1,M3,E1,P5);(A5,I1,M3,E1,P6);(A5,I1,M3,E1,P7);(A5,I1,M3,E1,P8);(A5,I1,M3,E1,P9);(A5,I1,M3,E1,P10);(A5,I1,M3,E1,P11);(A5,I1,M3,E1,P12);(A5,I1,M3,E1,P13);(A5,I1,M3,E1,P14);(A5,I1,M3,E1,P15);(A5,I1,M3,E1,P16);(A5,I1,M3,E1,P17);(A5,I1,M3,E1,P18);(A5,I1,M3,E1,P19);(A5,I1,M3,E1,P20);(A5,I1,M3,E2,P1);(A5,I1,M3,E2,P2);(A5,I1,M3,E2,P3);(A5,I1,M3,E2,P4);(A5,I1,M3,E2,P5);(A5,I1,M3,E2,P6);(A5,I1,M3,E2,P7);(A5,I1,M3,E2,P8);(A5,I1,M3,E2,P9);(A5,I1,M3,E2,P10);(A5,I1,M3,E2,P11);(A5,I1,M3,E2,P12);(A5,I1,M3,E2,P13);(A5,I1,M3,E2,P14);(A5,I1,M3,E2,P15);(A5,I1,M3,E2,P16);(A5,I1,M3,E2,P17);(A5,I1,M3,E2,P18);(A5,I1,M3,E2,P19);(A5,I1,M3,E2,P20);(A5,I1,M3,E3,P1);(A5,I1,M3,E3,P2);(A5,I1,M3,E3,P3);(A5,I1,M3,E3,P4);(A5,I1,M3,E3,P5);(A5,I1,M3,E3,P6);(A5,I1,M3,E3,P7);(A5,I1,M3,E3,P8);(A5,I1,M3,E3,P9);(A5,I1,M3,E3,P10);(A5,I1,M3,E3,P11);(A5,I1,M3,E3,P12);(A5,I1,M3,E3,P13);(A5,I1,M3,E3,P14);(A5,I1,M3,E3,P15);(A5,I1,M3,E3,P16);(A5,I1,M3,E3,P17);(A5,I1,M3,E3,P18);(A5,I1,M3,E3,P19);(A5,I1,M3,E3,P20);(A5,I1,M3,E4,P1);(A5,I1,M3,E4,P2);(A5,I1,M3,E4,P3);(A5,I1,M3,E4,P4);(A5,I1,M3,E4,P5);(A5,I1,M3,E4,P6);(A5,I1,M3,E4,P7);(A5,I1,M3,E4,P8);(A5,I1,M3,E4,P9);(A5,I1,M3,E4,P10);(A5,I1,M3,E4,P11);(A5,I1,M3,E4,P12);(A5,I1,M3,E4,P13);(A5,I1,M3,E4,P14);(A5,I1,M3,E4,P15);(A5,I1,M3,E4,P16);(A5,I1,M3,E4,P17);(A5,I1,M3,E4,P18);(A5,I1,M3,E4,P19);(A5,I1,M3,E4,P20);(A5,I1,M3,E5,P1);(A5,I1,M3,E5,P2);(A5,I1,M3,E5,P3);(A5,I1,M3,E5,P4);(A5,I1,M3,E5,P5);(A5,I1,M3,E5,P6);(A5,I1,M3,E5,P7);(A5,I1,M3,E5,P8);(A5,I1,M3,E5,P9);(A5,I1,M3,E5,P10);(A5,I1,M3,E5,P11);(A5,I1,M3,E5,P12);(A5,I1,M3,E5,P13);(A5,I1,M3,E5,P14);(A5,I1,M3,E5,P15);(A5,I1,M3,E5,P16);(A5,I1,M3,E5,P17);(A5,I1,M3,E5,P18);(A5,I1,M3,E5,P19);(A5,I1,M3,E5,P20);(A5,I1,M3,E6,P1);(A5,I1,M3,E6,P2);(A5,I1,M3,E6,P3);(A5,I1,M3,E6,P4);(A5,I1,M3,E6,P5);(A5,I1,M3,E6,P6);(A5,I1,M3,E6,P7);(A5,I1,M3,E6,P8);(A5,I1,M3,E6,P9);(A5,I1,M3,E6,P10);(A5,I1,M3,E6,P11);(A5,I1,M3,E6,P12);(A5,I1,M3,E6,P13);(A5,I1,M3,E6,P14);(A5,I1,M3,E6,P15);(A5,I1,M3,E6,P16);(A5,I1,M3,E6,P17);(A5,I1,M3,E6,P18);(A5,I1,M3,E6,P19);(A5,I1,M3,E6,P20);(A5,I1,M3,E7,P1);(A5,I1,M3,E7,P2);(A5,I1,M3,E7,P3);(A5,I1,M3,E7,P4);(A5,I1,M3,E7,P5);(A5,I1,M3,E7,P6);(A5,I1,M3,E7,P7);(A5,I1,M3,E7,P8);(A5,I1,M3,E7,P9);(A5,I1,M3,E7,P10);(A5,I1,M3,E7,P11);(A5,I1,M3,E7,P12);(A5,I1,M3,E7,P13);(A5,I1,M3,E7,P14);(A5,I1,M3,E7,P15);(A5,I1,M3,E7,P16);(A5,I1,M3,E7,P17);(A5,I1,M3,E7,P18);(A5,I1,M3,E7,P19);(A5,I1,M3,E7,P20);(A5,I1,M3,E8,P1);(A5,I1,M3,E8,P2);(A5,I1,M3,E8,P3);(A5,I1,M3,E8,P4);(A5,I1,M3,E8,P5);(A5,I1,M3,E8,P6);(A5,I1,M3,E8,P7);(A5,I1,M3,E8,P8);(A5,I1,M3,E8,P9);(A5,I1,M3,E8,P10);(A5,I1,M3,E8,P11);(A5,I1,M3,E8,P12);(A5,I1,M3,E8,P13);(A5,I1,M3,E8,P14);(A5,I1,M3,E8,P15);(A5,I1,M3,E8,P16);(A5,I1,M3,E8,P17);(A5,I1,M3,E8,P18);(A5,I1,M3,E8,P19);(A5,I1,M3,E8,P20);(A5,I1,M3,E9,P1);(A5,I1,M3,E9,P2);(A5,I1,M3,E9,P3);(A5,I1,M3,E9,P4);(A5,I1,M3,E9,P5);(A5,I1,M3,E9,P6);(A5,I1,M3,E9,P7);(A5,I1,M3,E9,P8);(A5,I1,M3,E9,P9);(A5,I1,M3,E9,P10);(A5,I1,M3,E9,P11);(A5,I1,M3,E9,P12);(A5,I1,M3,E9,P13);(A5,I1,M3,E9,P14);(A5,I1,M3,E9,P15);(A5,I1,M3,E9,P16);(A5,I1,M3,E9,P17);(A5,I1,M3,E9,P18);(A5,I1,M3,E9,P19);(A5,I1,M3,E9,P20);(A5,I1,M4,E1,P1);(A5,I1,M4,E1,P2);(A5,I1,M4,E1,P3);(A5,I1,M4,E1,P4);(A5,I1,M4,E1,P5);(A5,I1,M4,E1,P6);(A5,I1,M4,E1,P7);(A5,I1,M4,E1,P8);(A5,I1,M4,E1,P9);(A5,I1,M4,E1,P10);(A5,I1,M4,E1,P11);(A5,I1,M4,E1,P12);(A5,I1,M4,E1,P13);(A5,I1,M4,E1,P14);(A5,I1,M4,E1,P15);(A5,I1,M4,E1,P16);(A5,I1,M4,E1,P17);(A5,I1,M4,E1,P18);(A5,I1,M4,E1,P19);(A5,I1,M4,E1,P20);(A5,I1,M4,E2,P1);(A5,I1,M4,E2,P2);(A5,I1,M4,E2,P3);(A5,I1,M4,E2,P4);(A5,I1,M4,E2,P5);(A5,I1,M4,E2,P6);(A5,I1,M4,E2,P7);(A5,I1,M4,E2,P8);(A5,I1,M4,E2,P9);(A5,I1,M4,E2,P10);(A5,I1,M4,E2,P11);(A5,I1,M4,E2,P12);(A5,I1,M4,E2,P13);(A5,I1,M4,E2,P14);(A5,I1,M4,E2,P15);(A5,I1,M4,E2,P16);(A5,I1,M4,E2,P17);(A5,I1,M4,E2,P18);(A5,I1,M4,E2,P19);(A5,I1,M4,E2,P20);(A5,I1,M4,E3,P1);(A5,I1,M4,E3,P2);(A5,I1,M4,E3,P3);(A5,I1,M4,E3,P4);(A5,I1,M4,E3,P5);(A5,I1,M4,E3,P6);(A5,I1,M4,E3,P7);(A5,I1,M4,E3,P8);(A5,I1,M4,E3,P9);(A5,I1,M4,E3,P10);(A5,I1,M4,E3,P11);(A5,I1,M4,E3,P12);(A5,I1,M4,E3,P13);(A5,I1,M4,E3,P14);(A5,I1,M4,E3,P15);(A5,I1,M4,E3,P16);(A5,I1,M4,E3,P17);(A5,I1,M4,E3,P18);(A5,I1,M4,E3,P19);(A5,I1,M4,E3,P20);(A5,I1,M4,E4,P1);(A5,I1,M4,E4,P2);(A5,I1,M4,E4,P3);(A5,I1,M4,E4,P4);(A5,I1,M4,E4,P5);(A5,I1,M4,E4,P6);(A5,I1,M4,E4,P7);(A5,I1,M4,E4,P8);(A5,I1,M4,E4,P9);(A5,I1,M4,E4,P10);(A5,I1,M4,E4,P11);(A5,I1,M4,E4,P12);(A5,I1,M4,E4,P13);(A5,I1,M4,E4,P14);(A5,I1,M4,E4,P15);(A5,I1,M4,E4,P16);(A5,I1,M4,E4,P17);(A5,I1,M4,E4,P18);(A5,I1,M4,E4,P19);(A5,I1,M4,E4,P20);(A5,I1,M4,E5,P1);(A5,I1,M4,E5,P2);(A5,I1,M4,E5,P3);(A5,I1,M4,E5,P4);(A5,I1,M4,E5,P5);(A5,I1,M4,E5,P6);(A5,I1,M4,E5,P7);(A5,I1,M4,E5,P8);(A5,I1,M4,E5,P9);(A5,I1,M4,E5,P10);(A5,I1,M4,E5,P11);(A5,I1,M4,E5,P12);(A5,I1,M4,E5,P13);(A5,I1,M4,E5,P14);(A5,I1,M4,E5,P15);(A5,I1,M4,E5,P16);(A5,I1,M4,E5,P17);(A5,I1,M4,E5,P18);(A5,I1,M4,E5,P19);(A5,I1,M4,E5,P20);(A5,I1,M4,E6,P1);(A5,I1,M4,E6,P2);(A5,I1,M4,E6,P3);(A5,I1,M4,E6,P4);(A5,I1,M4,E6,P5);(A5,I1,M4,E6,P6);(A5,I1,M4,E6,P7);(A5,I1,M4,E6,P8);(A5,I1,M4,E6,P9);(A5,I1,M4,E6,P10);(A5,I1,M4,E6,P11);(A5,I1,M4,E6,P12);(A5,I1,M4,E6,P13);(A5,I1,M4,E6,P14);(A5,I1,M4,E6,P15);(A5,I1,M4,E6,P16);(A5,I1,M4,E6,P17);(A5,I1,M4,E6,P18);(A5,I1,M4,E6,P19);(A5,I1,M4,E6,P20);(A5,I1,M4,E7,P1);(A5,I1,M4,E7,P2);(A5,I1,M4,E7,P3);(A5,I1,M4,E7,P4);(A5,I1,M4,E7,P5);(A5,I1,M4,E7,P6);(A5,I1,M4,E7,P7);(A5,I1,M4,E7,P8);(A5,I1,M4,E7,P9);(A5,I1,M4,E7,P10);(A5,I1,M4,E7,P11);(A5,I1,M4,E7,P12);(A5,I1,M4,E7,P13);(A5,I1,M4,E7,P14);(A5,I1,M4,E7,P15);(A5,I1,M4,E7,P16);(A5,I1,M4,E7,P17);(A5,I1,M4,E7,P18);(A5,I1,M4,E7,P19);(A5,I1,M4,E7,P20);(A5,I1,M4,E8,P1);(A5,I1,M4,E8,P2);(A5,I1,M4,E8,P3);(A5,I1,M4,E8,P4);(A5,I1,M4,E8,P5);(A5,I1,M4,E8,P6);(A5,I1,M4,E8,P7);(A5,I1,M4,E8,P8);(A5,I1,M4,E8,P9);(A5,I1,M4,E8,P10);(A5,I1,M4,E8,P11);(A5,I1,M4,E8,P12);(A5,I1,M4,E8,P13);(A5,I1,M4,E8,P14);(A5,I1,M4,E8,P15);(A5,I1,M4,E8,P16);(A5,I1,M4,E8,P17);(A5,I1,M4,E8,P18);(A5,I1,M4,E8,P19);(A5,I1,M4,E8,P20);(A5,I1,M4,E9,P1);(A5,I1,M4,E9,P2);(A5,I1,M4,E9,P3);(A5,I1,M4,E9,P4);(A5,I1,M4,E9,P5);(A5,I1,M4,E9,P6);(A5,I1,M4,E9,P7);(A5,I1,M4,E9,P8);(A5,I1,M4,E9,P9);(A5,I1,M4,E9,P10);(A5,I1,M4,E9,P11);(A5,I1,M4,E9,P12);(A5,I1,M4,E9,P13);(A5,I1,M4,E9,P14);(A5,I1,M4,E9,P15);(A5,I1,M4,E9,P16);(A5,I1,M4,E9,P17);(A5,I1,M4,E9,P18);(A5,I1,M4,E9,P19);(A5,I1,M4,E9,P20);(A5,I1,M5,E1,P1);(A5,I1,M5,E1,P2);(A5,I1,M5,E1,P3);(A5,I1,M5,E1,P4);(A5,I1,M5,E1,P5);(A5,I1,M5,E1,P6);(A5,I1,M5,E1,P7);(A5,I1,M5,E1,P8);(A5,I1,M5,E1,P9);(A5,I1,M5,E1,P10);(A5,I1,M5,E1,P11);(A5,I1,M5,E1,P12);(A5,I1,M5,E1,P13);(A5,I1,M5,E1,P14);(A5,I1,M5,E1,P15);(A5,I1,M5,E1,P16);(A5,I1,M5,E1,P17);(A5,I1,M5,E1,P18);(A5,I1,M5,E1,P19);(A5,I1,M5,E1,P20);(A5,I1,M5,E2,P1);(A5,I1,M5,E2,P2);(A5,I1,M5,E2,P3);(A5,I1,M5,E2,P4);(A5,I1,M5,E2,P5);(A5,I1,M5,E2,P6);(A5,I1,M5,E2,P7);(A5,I1,M5,E2,P8);(A5,I1,M5,E2,P9);(A5,I1,M5,E2,P10);(A5,I1,M5,E2,P11);(A5,I1,M5,E2,P12);(A5,I1,M5,E2,P13);(A5,I1,M5,E2,P14);(A5,I1,M5,E2,P15);(A5,I1,M5,E2,P16);(A5,I1,M5,E2,P17);(A5,I1,M5,E2,P18);(A5,I1,M5,E2,P19);(A5,I1,M5,E2,P20);(A5,I1,M5,E3,P1);(A5,I1,M5,E3,P2);(A5,I1,M5,E3,P3);(A5,I1,M5,E3,P4);(A5,I1,M5,E3,P5);(A5,I1,M5,E3,P6);(A5,I1,M5,E3,P7);(A5,I1,M5,E3,P8);(A5,I1,M5,E3,P9);(A5,I1,M5,E3,P10);(A5,I1,M5,E3,P11);(A5,I1,M5,E3,P12);(A5,I1,M5,E3,P13);(A5,I1,M5,E3,P14);(A5,I1,M5,E3,P15);(A5,I1,M5,E3,P16);(A5,I1,M5,E3,P17);(A5,I1,M5,E3,P18);(A5,I1,M5,E3,P19);(A5,I1,M5,E3,P20);(A5,I1,M5,E4,P1);(A5,I1,M5,E4,P2);(A5,I1,M5,E4,P3);(A5,I1,M5,E4,P4);(A5,I1,M5,E4,P5);(A5,I1,M5,E4,P6);(A5,I1,M5,E4,P7);(A5,I1,M5,E4,P8);(A5,I1,M5,E4,P9);(A5,I1,M5,E4,P10);(A5,I1,M5,E4,P11);(A5,I1,M5,E4,P12);(A5,I1,M5,E4,P13);(A5,I1,M5,E4,P14);(A5,I1,M5,E4,P15);(A5,I1,M5,E4,P16);(A5,I1,M5,E4,P17);(A5,I1,M5,E4,P18);(A5,I1,M5,E4,P19);(A5,I1,M5,E4,P20);(A5,I1,M5,E5,P1);(A5,I1,M5,E5,P2);(A5,I1,M5,E5,P3);(A5,I1,M5,E5,P4);(A5,I1,M5,E5,P5);(A5,I1,M5,E5,P6);(A5,I1,M5,E5,P7);(A5,I1,M5,E5,P8);(A5,I1,M5,E5,P9);(A5,I1,M5,E5,P10);(A5,I1,M5,E5,P11);(A5,I1,M5,E5,P12);(A5,I1,M5,E5,P13);(A5,I1,M5,E5,P14);(A5,I1,M5,E5,P15);(A5,I1,M5,E5,P16);(A5,I1,M5,E5,P17);(A5,I1,M5,E5,P18);(A5,I1,M5,E5,P19);(A5,I1,M5,E5,P20);(A5,I1,M5,E6,P1);(A5,I1,M5,E6,P2);(A5,I1,M5,E6,P3);(A5,I1,M5,E6,P4);(A5,I1,M5,E6,P5);(A5,I1,M5,E6,P6);(A5,I1,M5,E6,P7);(A5,I1,M5,E6,P8);(A5,I1,M5,E6,P9);(A5,I1,M5,E6,P10);(A5,I1,M5,E6,P11);(A5,I1,M5,E6,P12);(A5,I1,M5,E6,P13);(A5,I1,M5,E6,P14);(A5,I1,M5,E6,P15);(A5,I1,M5,E6,P16);(A5,I1,M5,E6,P17);(A5,I1,M5,E6,P18);(A5,I1,M5,E6,P19);(A5,I1,M5,E6,P20);(A5,I1,M5,E7,P1);(A5,I1,M5,E7,P2);(A5,I1,M5,E7,P3);(A5,I1,M5,E7,P4);(A5,I1,M5,E7,P5);(A5,I1,M5,E7,P6);(A5,I1,M5,E7,P7);(A5,I1,M5,E7,P8);(A5,I1,M5,E7,P9);(A5,I1,M5,E7,P10);(A5,I1,M5,E7,P11);(A5,I1,M5,E7,P12);(A5,I1,M5,E7,P13);(A5,I1,M5,E7,P14);(A5,I1,M5,E7,P15);(A5,I1,M5,E7,P16);(A5,I1,M5,E7,P17);(A5,I1,M5,E7,P18);(A5,I1,M5,E7,P19);(A5,I1,M5,E7,P20);(A5,I1,M5,E8,P1);(A5,I1,M5,E8,P2);(A5,I1,M5,E8,P3);(A5,I1,M5,E8,P4);(A5,I1,M5,E8,P5);(A5,I1,M5,E8,P6);(A5,I1,M5,E8,P7);(A5,I1,M5,E8,P8);(A5,I1,M5,E8,P9);(A5,I1,M5,E8,P10);(A5,I1,M5,E8,P11);(A5,I1,M5,E8,P12);(A5,I1,M5,E8,P13);(A5,I1,M5,E8,P14);(A5,I1,M5,E8,P15);(A5,I1,M5,E8,P16);(A5,I1,M5,E8,P17);(A5,I1,M5,E8,P18);(A5,I1,M5,E8,P19);(A5,I1,M5,E8,P20);(A5,I1,M5,E9,P1);(A5,I1,M5,E9,P2);(A5,I1,M5,E9,P3);(A5,I1,M5,E9,P4);(A5,I1,M5,E9,P5);(A5,I1,M5,E9,P6);(A5,I1,M5,E9,P7);(A5,I1,M5,E9,P8);(A5,I1,M5,E9,P9);(A5,I1,M5,E9,P10);(A5,I1,M5,E9,P11);(A5,I1,M5,E9,P12);(A5,I1,M5,E9,P13);(A5,I1,M5,E9,P14);(A5,I1,M5,E9,P15);(A5,I1,M5,E9,P16);(A5,I1,M5,E9,P17);(A5,I1,M5,E9,P18);(A5,I1,M5,E9,P19);(A5,I1,M5,E9,P20);(A5,I1,M6,E1,P1);(A5,I1,M6,E1,P2);(A5,I1,M6,E1,P3);(A5,I1,M6,E1,P4);(A5,I1,M6,E1,P5);(A5,I1,M6,E1,P6);(A5,I1,M6,E1,P7);(A5,I1,M6,E1,P8);(A5,I1,M6,E1,P9);(A5,I1,M6,E1,P10);(A5,I1,M6,E1,P11);(A5,I1,M6,E1,P12);(A5,I1,M6,E1,P13);(A5,I1,M6,E1,P14);(A5,I1,M6,E1,P15);(A5,I1,M6,E1,P16);(A5,I1,M6,E1,P17);(A5,I1,M6,E1,P18);(A5,I1,M6,E1,P19);(A5,I1,M6,E1,P20);(A5,I1,M6,E2,P1);(A5,I1,M6,E2,P2);(A5,I1,M6,E2,P3);(A5,I1,M6,E2,P4);(A5,I1,M6,E2,P5);(A5,I1,M6,E2,P6);(A5,I1,M6,E2,P7);(A5,I1,M6,E2,P8);(A5,I1,M6,E2,P9);(A5,I1,M6,E2,P10);(A5,I1,M6,E2,P11);(A5,I1,M6,E2,P12);(A5,I1,M6,E2,P13);(A5,I1,M6,E2,P14);(A5,I1,M6,E2,P15);(A5,I1,M6,E2,P16);(A5,I1,M6,E2,P17);(A5,I1,M6,E2,P18);(A5,I1,M6,E2,P19);(A5,I1,M6,E2,P20);(A5,I1,M6,E3,P1);(A5,I1,M6,E3,P2);(A5,I1,M6,E3,P3);(A5,I1,M6,E3,P4);(A5,I1,M6,E3,P5);(A5,I1,M6,E3,P6);(A5,I1,M6,E3,P7);(A5,I1,M6,E3,P8);(A5,I1,M6,E3,P9);(A5,I1,M6,E3,P10);(A5,I1,M6,E3,P11);(A5,I1,M6,E3,P12);(A5,I1,M6,E3,P13);(A5,I1,M6,E3,P14);(A5,I1,M6,E3,P15);(A5,I1,M6,E3,P16);(A5,I1,M6,E3,P17);(A5,I1,M6,E3,P18);(A5,I1,M6,E3,P19);(A5,I1,M6,E3,P20);(A5,I1,M6,E4,P1);(A5,I1,M6,E4,P2);(A5,I1,M6,E4,P3);(A5,I1,M6,E4,P4);(A5,I1,M6,E4,P5);(A5,I1,M6,E4,P6);(A5,I1,M6,E4,P7);(A5,I1,M6,E4,P8);(A5,I1,M6,E4,P9);(A5,I1,M6,E4,P10);(A5,I1,M6,E4,P11);(A5,I1,M6,E4,P12);(A5,I1,M6,E4,P13);(A5,I1,M6,E4,P14);(A5,I1,M6,E4,P15);(A5,I1,M6,E4,P16);(A5,I1,M6,E4,P17);(A5,I1,M6,E4,P18);(A5,I1,M6,E4,P19);(A5,I1,M6,E4,P20);(A5,I1,M6,E5,P1);(A5,I1,M6,E5,P2);(A5,I1,M6,E5,P3);(A5,I1,M6,E5,P4);(A5,I1,M6,E5,P5);(A5,I1,M6,E5,P6);(A5,I1,M6,E5,P7);(A5,I1,M6,E5,P8);(A5,I1,M6,E5,P9);(A5,I1,M6,E5,P10);(A5,I1,M6,E5,P11);(A5,I1,M6,E5,P12);(A5,I1,M6,E5,P13);(A5,I1,M6,E5,P14);(A5,I1,M6,E5,P15);(A5,I1,M6,E5,P16);(A5,I1,M6,E5,P17);(A5,I1,M6,E5,P18);(A5,I1,M6,E5,P19);(A5,I1,M6,E5,P20);(A5,I1,M6,E6,P1);(A5,I1,M6,E6,P2);(A5,I1,M6,E6,P3);(A5,I1,M6,E6,P4);(A5,I1,M6,E6,P5);(A5,I1,M6,E6,P6);(A5,I1,M6,E6,P7);(A5,I1,M6,E6,P8);(A5,I1,M6,E6,P9);(A5,I1,M6,E6,P10);(A5,I1,M6,E6,P11);(A5,I1,M6,E6,P12);(A5,I1,M6,E6,P13);(A5,I1,M6,E6,P14);(A5,I1,M6,E6,P15);(A5,I1,M6,E6,P16);(A5,I1,M6,E6,P17);(A5,I1,M6,E6,P18);(A5,I1,M6,E6,P19);(A5,I1,M6,E6,P20);(A5,I1,M6,E7,P1);(A5,I1,M6,E7,P2);(A5,I1,M6,E7,P3);(A5,I1,M6,E7,P4);(A5,I1,M6,E7,P5);(A5,I1,M6,E7,P6);(A5,I1,M6,E7,P7);(A5,I1,M6,E7,P8);(A5,I1,M6,E7,P9);(A5,I1,M6,E7,P10);(A5,I1,M6,E7,P11);(A5,I1,M6,E7,P12);(A5,I1,M6,E7,P13);(A5,I1,M6,E7,P14);(A5,I1,M6,E7,P15);(A5,I1,M6,E7,P16);(A5,I1,M6,E7,P17);(A5,I1,M6,E7,P18);(A5,I1,M6,E7,P19);(A5,I1,M6,E7,P20);(A5,I1,M6,E8,P1);(A5,I1,M6,E8,P2);(A5,I1,M6,E8,P3);(A5,I1,M6,E8,P4);(A5,I1,M6,E8,P5);(A5,I1,M6,E8,P6);(A5,I1,M6,E8,P7);(A5,I1,M6,E8,P8);(A5,I1,M6,E8,P9);(A5,I1,M6,E8,P10);(A5,I1,M6,E8,P11);(A5,I1,M6,E8,P12);(A5,I1,M6,E8,P13);(A5,I1,M6,E8,P14);(A5,I1,M6,E8,P15);(A5,I1,M6,E8,P16);(A5,I1,M6,E8,P17);(A5,I1,M6,E8,P18);(A5,I1,M6,E8,P19);(A5,I1,M6,E8,P20);(A5,I1,M6,E9,P1);(A5,I1,M6,E9,P2);(A5,I1,M6,E9,P3);(A5,I1,M6,E9,P4);(A5,I1,M6,E9,P5);(A5,I1,M6,E9,P6);(A5,I1,M6,E9,P7);(A5,I1,M6,E9,P8);(A5,I1,M6,E9,P9);(A5,I1,M6,E9,P10);(A5,I1,M6,E9,P11);(A5,I1,M6,E9,P12);(A5,I1,M6,E9,P13);(A5,I1,M6,E9,P14);(A5,I1,M6,E9,P15);(A5,I1,M6,E9,P16);(A5,I1,M6,E9,P17);(A5,I1,M6,E9,P18);(A5,I1,M6,E9,P19);(A5,I1,M6,E9,P20);(A5,I2,M1,E1,P1);(A5,I2,M1,E1,P2);(A5,I2,M1,E1,P3);(A5,I2,M1,E1,P4);(A5,I2,M1,E1,P5);(A5,I2,M1,E1,P6);(A5,I2,M1,E1,P7);(A5,I2,M1,E1,P8);(A5,I2,M1,E1,P9);(A5,I2,M1,E1,P10);(A5,I2,M1,E1,P11);(A5,I2,M1,E1,P12);(A5,I2,M1,E1,P13);(A5,I2,M1,E1,P14);(A5,I2,M1,E1,P15);(A5,I2,M1,E1,P16);(A5,I2,M1,E1,P17);(A5,I2,M1,E1,P18);(A5,I2,M1,E1,P19);(A5,I2,M1,E1,P20);(A5,I2,M1,E2,P1);(A5,I2,M1,E2,P2);(A5,I2,M1,E2,P3);(A5,I2,M1,E2,P4);(A5,I2,M1,E2,P5);(A5,I2,M1,E2,P6);(A5,I2,M1,E2,P7);(A5,I2,M1,E2,P8);(A5,I2,M1,E2,P9);(A5,I2,M1,E2,P10);(A5,I2,M1,E2,P11);(A5,I2,M1,E2,P12);(A5,I2,M1,E2,P13);(A5,I2,M1,E2,P14);(A5,I2,M1,E2,P15);(A5,I2,M1,E2,P16);(A5,I2,M1,E2,P17);(A5,I2,M1,E2,P18);(A5,I2,M1,E2,P19);(A5,I2,M1,E2,P20);(A5,I2,M1,E3,P1);(A5,I2,M1,E3,P2);(A5,I2,M1,E3,P3);(A5,I2,M1,E3,P4);(A5,I2,M1,E3,P5);(A5,I2,M1,E3,P6);(A5,I2,M1,E3,P7);(A5,I2,M1,E3,P8);(A5,I2,M1,E3,P9);(A5,I2,M1,E3,P10);(A5,I2,M1,E3,P11);(A5,I2,M1,E3,P12);(A5,I2,M1,E3,P13);(A5,I2,M1,E3,P14);(A5,I2,M1,E3,P15);(A5,I2,M1,E3,P16);(A5,I2,M1,E3,P17);(A5,I2,M1,E3,P18);(A5,I2,M1,E3,P19);(A5,I2,M1,E3,P20);(A5,I2,M1,E4,P1);(A5,I2,M1,E4,P2);(A5,I2,M1,E4,P3);(A5,I2,M1,E4,P4);(A5,I2,M1,E4,P5);(A5,I2,M1,E4,P6);(A5,I2,M1,E4,P7);(A5,I2,M1,E4,P8);(A5,I2,M1,E4,P9);(A5,I2,M1,E4,P10);(A5,I2,M1,E4,P11);(A5,I2,M1,E4,P12);(A5,I2,M1,E4,P13);(A5,I2,M1,E4,P14);(A5,I2,M1,E4,P15);(A5,I2,M1,E4,P16);(A5,I2,M1,E4,P17);(A5,I2,M1,E4,P18);(A5,I2,M1,E4,P19);(A5,I2,M1,E4,P20);(A5,I2,M1,E5,P1);(A5,I2,M1,E5,P2);(A5,I2,M1,E5,P3);(A5,I2,M1,E5,P4);(A5,I2,M1,E5,P5);(A5,I2,M1,E5,P6);(A5,I2,M1,E5,P7);(A5,I2,M1,E5,P8);(A5,I2,M1,E5,P9);(A5,I2,M1,E5,P10);(A5,I2,M1,E5,P11);(A5,I2,M1,E5,P12);(A5,I2,M1,E5,P13);(A5,I2,M1,E5,P14);(A5,I2,M1,E5,P15);(A5,I2,M1,E5,P16);(A5,I2,M1,E5,P17);(A5,I2,M1,E5,P18);(A5,I2,M1,E5,P19);(A5,I2,M1,E5,P20);(A5,I2,M1,E6,P1);(A5,I2,M1,E6,P2);(A5,I2,M1,E6,P3);(A5,I2,M1,E6,P4);(A5,I2,M1,E6,P5);(A5,I2,M1,E6,P6);(A5,I2,M1,E6,P7);(A5,I2,M1,E6,P8);(A5,I2,M1,E6,P9);(A5,I2,M1,E6,P10);(A5,I2,M1,E6,P11);(A5,I2,M1,E6,P12);(A5,I2,M1,E6,P13);(A5,I2,M1,E6,P14);(A5,I2,M1,E6,P15);(A5,I2,M1,E6,P16);(A5,I2,M1,E6,P17);(A5,I2,M1,E6,P18);(A5,I2,M1,E6,P19);(A5,I2,M1,E6,P20);(A5,I2,M1,E7,P1);(A5,I2,M1,E7,P2);(A5,I2,M1,E7,P3);(A5,I2,M1,E7,P4);(A5,I2,M1,E7,P5);(A5,I2,M1,E7,P6);(A5,I2,M1,E7,P7);(A5,I2,M1,E7,P8);(A5,I2,M1,E7,P9);(A5,I2,M1,E7,P10);(A5,I2,M1,E7,P11);(A5,I2,M1,E7,P12);(A5,I2,M1,E7,P13);(A5,I2,M1,E7,P14);(A5,I2,M1,E7,P15);(A5,I2,M1,E7,P16);(A5,I2,M1,E7,P17);(A5,I2,M1,E7,P18);(A5,I2,M1,E7,P19);(A5,I2,M1,E7,P20);(A5,I2,M1,E8,P1);(A5,I2,M1,E8,P2);(A5,I2,M1,E8,P3);(A5,I2,M1,E8,P4);(A5,I2,M1,E8,P5);(A5,I2,M1,E8,P6);(A5,I2,M1,E8,P7);(A5,I2,M1,E8,P8);(A5,I2,M1,E8,P9);(A5,I2,M1,E8,P10);(A5,I2,M1,E8,P11);(A5,I2,M1,E8,P12);(A5,I2,M1,E8,P13);(A5,I2,M1,E8,P14);(A5,I2,M1,E8,P15);(A5,I2,M1,E8,P16);(A5,I2,M1,E8,P17);(A5,I2,M1,E8,P18);(A5,I2,M1,E8,P19);(A5,I2,M1,E8,P20);(A5,I2,M1,E9,P1);(A5,I2,M1,E9,P2);(A5,I2,M1,E9,P3);(A5,I2,M1,E9,P4);(A5,I2,M1,E9,P5);(A5,I2,M1,E9,P6);(A5,I2,M1,E9,P7);(A5,I2,M1,E9,P8);(A5,I2,M1,E9,P9);(A5,I2,M1,E9,P10);(A5,I2,M1,E9,P11);(A5,I2,M1,E9,P12);(A5,I2,M1,E9,P13);(A5,I2,M1,E9,P14);(A5,I2,M1,E9,P15);(A5,I2,M1,E9,P16);(A5,I2,M1,E9,P17);(A5,I2,M1,E9,P18);(A5,I2,M1,E9,P19);(A5,I2,M1,E9,P20);(A5,I2,M2,E1,P1);(A5,I2,M2,E1,P2);(A5,I2,M2,E1,P3);(A5,I2,M2,E1,P4);(A5,I2,M2,E1,P5);(A5,I2,M2,E1,P6);(A5,I2,M2,E1,P7);(A5,I2,M2,E1,P8);(A5,I2,M2,E1,P9);(A5,I2,M2,E1,P10);(A5,I2,M2,E1,P11);(A5,I2,M2,E1,P12);(A5,I2,M2,E1,P13);(A5,I2,M2,E1,P14);(A5,I2,M2,E1,P15);(A5,I2,M2,E1,P16);(A5,I2,M2,E1,P17);(A5,I2,M2,E1,P18);(A5,I2,M2,E1,P19);(A5,I2,M2,E1,P20);(A5,I2,M2,E2,P1);(A5,I2,M2,E2,P2);(A5,I2,M2,E2,P3);(A5,I2,M2,E2,P4);(A5,I2,M2,E2,P5);(A5,I2,M2,E2,P6);(A5,I2,M2,E2,P7);(A5,I2,M2,E2,P8);(A5,I2,M2,E2,P9);(A5,I2,M2,E2,P10);(A5,I2,M2,E2,P11);(A5,I2,M2,E2,P12);(A5,I2,M2,E2,P13);(A5,I2,M2,E2,P14);(A5,I2,M2,E2,P15);(A5,I2,M2,E2,P16);(A5,I2,M2,E2,P17);(A5,I2,M2,E2,P18);(A5,I2,M2,E2,P19);(A5,I2,M2,E2,P20);(A5,I2,M2,E3,P1);(A5,I2,M2,E3,P2);(A5,I2,M2,E3,P3);(A5,I2,M2,E3,P4);(A5,I2,M2,E3,P5);(A5,I2,M2,E3,P6);(A5,I2,M2,E3,P7);(A5,I2,M2,E3,P8);(A5,I2,M2,E3,P9);(A5,I2,M2,E3,P10);(A5,I2,M2,E3,P11);(A5,I2,M2,E3,P12);(A5,I2,M2,E3,P13);(A5,I2,M2,E3,P14);(A5,I2,M2,E3,P15);(A5,I2,M2,E3,P16);(A5,I2,M2,E3,P17);(A5,I2,M2,E3,P18);(A5,I2,M2,E3,P19);(A5,I2,M2,E3,P20);(A5,I2,M2,E4,P1);(A5,I2,M2,E4,P2);(A5,I2,M2,E4,P3);(A5,I2,M2,E4,P4);(A5,I2,M2,E4,P5);(A5,I2,M2,E4,P6);(A5,I2,M2,E4,P7);(A5,I2,M2,E4,P8);(A5,I2,M2,E4,P9);(A5,I2,M2,E4,P10);(A5,I2,M2,E4,P11);(A5,I2,M2,E4,P12);(A5,I2,M2,E4,P13);(A5,I2,M2,E4,P14);(A5,I2,M2,E4,P15);(A5,I2,M2,E4,P16);(A5,I2,M2,E4,P17);(A5,I2,M2,E4,P18);(A5,I2,M2,E4,P19);(A5,I2,M2,E4,P20);(A5,I2,M2,E5,P1);(A5,I2,M2,E5,P2);(A5,I2,M2,E5,P3);(A5,I2,M2,E5,P4);(A5,I2,M2,E5,P5);(A5,I2,M2,E5,P6);(A5,I2,M2,E5,P7);(A5,I2,M2,E5,P8);(A5,I2,M2,E5,P9);(A5,I2,M2,E5,P10);(A5,I2,M2,E5,P11);(A5,I2,M2,E5,P12);(A5,I2,M2,E5,P13);(A5,I2,M2,E5,P14);(A5,I2,M2,E5,P15);(A5,I2,M2,E5,P16);(A5,I2,M2,E5,P17);(A5,I2,M2,E5,P18);(A5,I2,M2,E5,P19);(A5,I2,M2,E5,P20);(A5,I2,M2,E6,P1);(A5,I2,M2,E6,P2);(A5,I2,M2,E6,P3);(A5,I2,M2,E6,P4);(A5,I2,M2,E6,P5);(A5,I2,M2,E6,P6);(A5,I2,M2,E6,P7);(A5,I2,M2,E6,P8);(A5,I2,M2,E6,P9);(A5,I2,M2,E6,P10);(A5,I2,M2,E6,P11);(A5,I2,M2,E6,P12);(A5,I2,M2,E6,P13);(A5,I2,M2,E6,P14);(A5,I2,M2,E6,P15);(A5,I2,M2,E6,P16);(A5,I2,M2,E6,P17);(A5,I2,M2,E6,P18);(A5,I2,M2,E6,P19);(A5,I2,M2,E6,P20);(A5,I2,M2,E7,P1);(A5,I2,M2,E7,P2);(A5,I2,M2,E7,P3);(A5,I2,M2,E7,P4);(A5,I2,M2,E7,P5);(A5,I2,M2,E7,P6);(A5,I2,M2,E7,P7);(A5,I2,M2,E7,P8);(A5,I2,M2,E7,P9);(A5,I2,M2,E7,P10);(A5,I2,M2,E7,P11);(A5,I2,M2,E7,P12);(A5,I2,M2,E7,P13);(A5,I2,M2,E7,P14);(A5,I2,M2,E7,P15);(A5,I2,M2,E7,P16);(A5,I2,M2,E7,P17);(A5,I2,M2,E7,P18);(A5,I2,M2,E7,P19);(A5,I2,M2,E7,P20);(A5,I2,M2,E8,P1);(A5,I2,M2,E8,P2);(A5,I2,M2,E8,P3);(A5,I2,M2,E8,P4);(A5,I2,M2,E8,P5);(A5,I2,M2,E8,P6);(A5,I2,M2,E8,P7);(A5,I2,M2,E8,P8);(A5,I2,M2,E8,P9);(A5,I2,M2,E8,P10);(A5,I2,M2,E8,P11);(A5,I2,M2,E8,P12);(A5,I2,M2,E8,P13);(A5,I2,M2,E8,P14);(A5,I2,M2,E8,P15);(A5,I2,M2,E8,P16);(A5,I2,M2,E8,P17);(A5,I2,M2,E8,P18);(A5,I2,M2,E8,P19);(A5,I2,M2,E8,P20);(A5,I2,M2,E9,P1);(A5,I2,M2,E9,P2);(A5,I2,M2,E9,P3);(A5,I2,M2,E9,P4);(A5,I2,M2,E9,P5);(A5,I2,M2,E9,P6);(A5,I2,M2,E9,P7);(A5,I2,M2,E9,P8);(A5,I2,M2,E9,P9);(A5,I2,M2,E9,P10);(A5,I2,M2,E9,P11);(A5,I2,M2,E9,P12);(A5,I2,M2,E9,P13);(A5,I2,M2,E9,P14);(A5,I2,M2,E9,P15);(A5,I2,M2,E9,P16);(A5,I2,M2,E9,P17);(A5,I2,M2,E9,P18);(A5,I2,M2,E9,P19);(A5,I2,M2,E9,P20);(A5,I2,M3,E1,P1);(A5,I2,M3,E1,P2);(A5,I2,M3,E1,P3);(A5,I2,M3,E1,P4);(A5,I2,M3,E1,P5);(A5,I2,M3,E1,P6);(A5,I2,M3,E1,P7);(A5,I2,M3,E1,P8);(A5,I2,M3,E1,P9);(A5,I2,M3,E1,P10);(A5,I2,M3,E1,P11);(A5,I2,M3,E1,P12);(A5,I2,M3,E1,P13);(A5,I2,M3,E1,P14);(A5,I2,M3,E1,P15);(A5,I2,M3,E1,P16);(A5,I2,M3,E1,P17);(A5,I2,M3,E1,P18);(A5,I2,M3,E1,P19);(A5,I2,M3,E1,P20);(A5,I2,M3,E2,P1);(A5,I2,M3,E2,P2);(A5,I2,M3,E2,P3);(A5,I2,M3,E2,P4);(A5,I2,M3,E2,P5);(A5,I2,M3,E2,P6);(A5,I2,M3,E2,P7);(A5,I2,M3,E2,P8);(A5,I2,M3,E2,P9);(A5,I2,M3,E2,P10);(A5,I2,M3,E2,P11);(A5,I2,M3,E2,P12);(A5,I2,M3,E2,P13);(A5,I2,M3,E2,P14);(A5,I2,M3,E2,P15);(A5,I2,M3,E2,P16);(A5,I2,M3,E2,P17);(A5,I2,M3,E2,P18);(A5,I2,M3,E2,P19);(A5,I2,M3,E2,P20);(A5,I2,M3,E3,P1);(A5,I2,M3,E3,P2);(A5,I2,M3,E3,P3);(A5,I2,M3,E3,P4);(A5,I2,M3,E3,P5);(A5,I2,M3,E3,P6);(A5,I2,M3,E3,P7);(A5,I2,M3,E3,P8);(A5,I2,M3,E3,P9);(A5,I2,M3,E3,P10);(A5,I2,M3,E3,P11);(A5,I2,M3,E3,P12);(A5,I2,M3,E3,P13);(A5,I2,M3,E3,P14);(A5,I2,M3,E3,P15);(A5,I2,M3,E3,P16);(A5,I2,M3,E3,P17);(A5,I2,M3,E3,P18);(A5,I2,M3,E3,P19);(A5,I2,M3,E3,P20);(A5,I2,M3,E4,P1);(A5,I2,M3,E4,P2);(A5,I2,M3,E4,P3);(A5,I2,M3,E4,P4);(A5,I2,M3,E4,P5);(A5,I2,M3,E4,P6);(A5,I2,M3,E4,P7);(A5,I2,M3,E4,P8);(A5,I2,M3,E4,P9);(A5,I2,M3,E4,P10);(A5,I2,M3,E4,P11);(A5,I2,M3,E4,P12);(A5,I2,M3,E4,P13);(A5,I2,M3,E4,P14);(A5,I2,M3,E4,P15);(A5,I2,M3,E4,P16);(A5,I2,M3,E4,P17);(A5,I2,M3,E4,P18);(A5,I2,M3,E4,P19);(A5,I2,M3,E4,P20);(A5,I2,M3,E5,P1);(A5,I2,M3,E5,P2);(A5,I2,M3,E5,P3);(A5,I2,M3,E5,P4);(A5,I2,M3,E5,P5);(A5,I2,M3,E5,P6);(A5,I2,M3,E5,P7);(A5,I2,M3,E5,P8);(A5,I2,M3,E5,P9);(A5,I2,M3,E5,P10);(A5,I2,M3,E5,P11);(A5,I2,M3,E5,P12);(A5,I2,M3,E5,P13);(A5,I2,M3,E5,P14);(A5,I2,M3,E5,P15);(A5,I2,M3,E5,P16);(A5,I2,M3,E5,P17);(A5,I2,M3,E5,P18);(A5,I2,M3,E5,P19);(A5,I2,M3,E5,P20);(A5,I2,M3,E6,P1);(A5,I2,M3,E6,P2);(A5,I2,M3,E6,P3);(A5,I2,M3,E6,P4);(A5,I2,M3,E6,P5);(A5,I2,M3,E6,P6);(A5,I2,M3,E6,P7);(A5,I2,M3,E6,P8);(A5,I2,M3,E6,P9);(A5,I2,M3,E6,P10);(A5,I2,M3,E6,P11);(A5,I2,M3,E6,P12);(A5,I2,M3,E6,P13);(A5,I2,M3,E6,P14);(A5,I2,M3,E6,P15);(A5,I2,M3,E6,P16);(A5,I2,M3,E6,P17);(A5,I2,M3,E6,P18);(A5,I2,M3,E6,P19);(A5,I2,M3,E6,P20);(A5,I2,M3,E7,P1);(A5,I2,M3,E7,P2);(A5,I2,M3,E7,P3);(A5,I2,M3,E7,P4);(A5,I2,M3,E7,P5);(A5,I2,M3,E7,P6);(A5,I2,M3,E7,P7);(A5,I2,M3,E7,P8);(A5,I2,M3,E7,P9);(A5,I2,M3,E7,P10);(A5,I2,M3,E7,P11);(A5,I2,M3,E7,P12);(A5,I2,M3,E7,P13);(A5,I2,M3,E7,P14);(A5,I2,M3,E7,P15);(A5,I2,M3,E7,P16);(A5,I2,M3,E7,P17);(A5,I2,M3,E7,P18);(A5,I2,M3,E7,P19);(A5,I2,M3,E7,P20);(A5,I2,M3,E8,P1);(A5,I2,M3,E8,P2);(A5,I2,M3,E8,P3);(A5,I2,M3,E8,P4);(A5,I2,M3,E8,P5);(A5,I2,M3,E8,P6);(A5,I2,M3,E8,P7);(A5,I2,M3,E8,P8);(A5,I2,M3,E8,P9);(A5,I2,M3,E8,P10);(A5,I2,M3,E8,P11);(A5,I2,M3,E8,P12);(A5,I2,M3,E8,P13);(A5,I2,M3,E8,P14);(A5,I2,M3,E8,P15);(A5,I2,M3,E8,P16);(A5,I2,M3,E8,P17);(A5,I2,M3,E8,P18);(A5,I2,M3,E8,P19);(A5,I2,M3,E8,P20);(A5,I2,M3,E9,P1);(A5,I2,M3,E9,P2);(A5,I2,M3,E9,P3);(A5,I2,M3,E9,P4);(A5,I2,M3,E9,P5);(A5,I2,M3,E9,P6);(A5,I2,M3,E9,P7);(A5,I2,M3,E9,P8);(A5,I2,M3,E9,P9);(A5,I2,M3,E9,P10);(A5,I2,M3,E9,P11);(A5,I2,M3,E9,P12);(A5,I2,M3,E9,P13);(A5,I2,M3,E9,P14);(A5,I2,M3,E9,P15);(A5,I2,M3,E9,P16);(A5,I2,M3,E9,P17);(A5,I2,M3,E9,P18);(A5,I2,M3,E9,P19);(A5,I2,M3,E9,P20);(A5,I2,M4,E1,P1);(A5,I2,M4,E1,P2);(A5,I2,M4,E1,P3);(A5,I2,M4,E1,P4);(A5,I2,M4,E1,P5);(A5,I2,M4,E1,P6);(A5,I2,M4,E1,P7);(A5,I2,M4,E1,P8);(A5,I2,M4,E1,P9);(A5,I2,M4,E1,P10);(A5,I2,M4,E1,P11);(A5,I2,M4,E1,P12);(A5,I2,M4,E1,P13);(A5,I2,M4,E1,P14);(A5,I2,M4,E1,P15);(A5,I2,M4,E1,P16);(A5,I2,M4,E1,P17);(A5,I2,M4,E1,P18);(A5,I2,M4,E1,P19);(A5,I2,M4,E1,P20);(A5,I2,M4,E2,P1);(A5,I2,M4,E2,P2);(A5,I2,M4,E2,P3);(A5,I2,M4,E2,P4);(A5,I2,M4,E2,P5);(A5,I2,M4,E2,P6);(A5,I2,M4,E2,P7);(A5,I2,M4,E2,P8);(A5,I2,M4,E2,P9);(A5,I2,M4,E2,P10);(A5,I2,M4,E2,P11);(A5,I2,M4,E2,P12);(A5,I2,M4,E2,P13);(A5,I2,M4,E2,P14);(A5,I2,M4,E2,P15);(A5,I2,M4,E2,P16);(A5,I2,M4,E2,P17);(A5,I2,M4,E2,P18);(A5,I2,M4,E2,P19);(A5,I2,M4,E2,P20);(A5,I2,M4,E3,P1);(A5,I2,M4,E3,P2);(A5,I2,M4,E3,P3);(A5,I2,M4,E3,P4);(A5,I2,M4,E3,P5);(A5,I2,M4,E3,P6);(A5,I2,M4,E3,P7);(A5,I2,M4,E3,P8);(A5,I2,M4,E3,P9);(A5,I2,M4,E3,P10);(A5,I2,M4,E3,P11);(A5,I2,M4,E3,P12);(A5,I2,M4,E3,P13);(A5,I2,M4,E3,P14);(A5,I2,M4,E3,P15);(A5,I2,M4,E3,P16);(A5,I2,M4,E3,P17);(A5,I2,M4,E3,P18);(A5,I2,M4,E3,P19);(A5,I2,M4,E3,P20);(A5,I2,M4,E4,P1);(A5,I2,M4,E4,P2);(A5,I2,M4,E4,P3);(A5,I2,M4,E4,P4);(A5,I2,M4,E4,P5);(A5,I2,M4,E4,P6);(A5,I2,M4,E4,P7);(A5,I2,M4,E4,P8);(A5,I2,M4,E4,P9);(A5,I2,M4,E4,P10);(A5,I2,M4,E4,P11);(A5,I2,M4,E4,P12);(A5,I2,M4,E4,P13);(A5,I2,M4,E4,P14);(A5,I2,M4,E4,P15);(A5,I2,M4,E4,P16);(A5,I2,M4,E4,P17);(A5,I2,M4,E4,P18);(A5,I2,M4,E4,P19);(A5,I2,M4,E4,P20);(A5,I2,M4,E5,P1);(A5,I2,M4,E5,P2);(A5,I2,M4,E5,P3);(A5,I2,M4,E5,P4);(A5,I2,M4,E5,P5);(A5,I2,M4,E5,P6);(A5,I2,M4,E5,P7);(A5,I2,M4,E5,P8);(A5,I2,M4,E5,P9);(A5,I2,M4,E5,P10);(A5,I2,M4,E5,P11);(A5,I2,M4,E5,P12);(A5,I2,M4,E5,P13);(A5,I2,M4,E5,P14);(A5,I2,M4,E5,P15);(A5,I2,M4,E5,P16);(A5,I2,M4,E5,P17);(A5,I2,M4,E5,P18);(A5,I2,M4,E5,P19);(A5,I2,M4,E5,P20);(A5,I2,M4,E6,P1);(A5,I2,M4,E6,P2);(A5,I2,M4,E6,P3);(A5,I2,M4,E6,P4);(A5,I2,M4,E6,P5);(A5,I2,M4,E6,P6);(A5,I2,M4,E6,P7);(A5,I2,M4,E6,P8);(A5,I2,M4,E6,P9);(A5,I2,M4,E6,P10);(A5,I2,M4,E6,P11);(A5,I2,M4,E6,P12);(A5,I2,M4,E6,P13);(A5,I2,M4,E6,P14);(A5,I2,M4,E6,P15);(A5,I2,M4,E6,P16);(A5,I2,M4,E6,P17);(A5,I2,M4,E6,P18);(A5,I2,M4,E6,P19);(A5,I2,M4,E6,P20);(A5,I2,M4,E7,P1);(A5,I2,M4,E7,P2);(A5,I2,M4,E7,P3);(A5,I2,M4,E7,P4);(A5,I2,M4,E7,P5);(A5,I2,M4,E7,P6);(A5,I2,M4,E7,P7);(A5,I2,M4,E7,P8);(A5,I2,M4,E7,P9);(A5,I2,M4,E7,P10);(A5,I2,M4,E7,P11);(A5,I2,M4,E7,P12);(A5,I2,M4,E7,P13);(A5,I2,M4,E7,P14);(A5,I2,M4,E7,P15);(A5,I2,M4,E7,P16);(A5,I2,M4,E7,P17);(A5,I2,M4,E7,P18);(A5,I2,M4,E7,P19);(A5,I2,M4,E7,P20);(A5,I2,M4,E8,P1);(A5,I2,M4,E8,P2);(A5,I2,M4,E8,P3);(A5,I2,M4,E8,P4);(A5,I2,M4,E8,P5);(A5,I2,M4,E8,P6);(A5,I2,M4,E8,P7);(A5,I2,M4,E8,P8);(A5,I2,M4,E8,P9);(A5,I2,M4,E8,P10);(A5,I2,M4,E8,P11);(A5,I2,M4,E8,P12);(A5,I2,M4,E8,P13);(A5,I2,M4,E8,P14);(A5,I2,M4,E8,P15);(A5,I2,M4,E8,P16);(A5,I2,M4,E8,P17);(A5,I2,M4,E8,P18);(A5,I2,M4,E8,P19);(A5,I2,M4,E8,P20);(A5,I2,M4,E9,P1);(A5,I2,M4,E9,P2);(A5,I2,M4,E9,P3);(A5,I2,M4,E9,P4);(A5,I2,M4,E9,P5);(A5,I2,M4,E9,P6);(A5,I2,M4,E9,P7);(A5,I2,M4,E9,P8);(A5,I2,M4,E9,P9);(A5,I2,M4,E9,P10);(A5,I2,M4,E9,P11);(A5,I2,M4,E9,P12);(A5,I2,M4,E9,P13);(A5,I2,M4,E9,P14);(A5,I2,M4,E9,P15);(A5,I2,M4,E9,P16);(A5,I2,M4,E9,P17);(A5,I2,M4,E9,P18);(A5,I2,M4,E9,P19);(A5,I2,M4,E9,P20);(A5,I2,M5,E1,P1);(A5,I2,M5,E1,P2);(A5,I2,M5,E1,P3);(A5,I2,M5,E1,P4);(A5,I2,M5,E1,P5);(A5,I2,M5,E1,P6);(A5,I2,M5,E1,P7);(A5,I2,M5,E1,P8);(A5,I2,M5,E1,P9);(A5,I2,M5,E1,P10);(A5,I2,M5,E1,P11);(A5,I2,M5,E1,P12);(A5,I2,M5,E1,P13);(A5,I2,M5,E1,P14);(A5,I2,M5,E1,P15);(A5,I2,M5,E1,P16);(A5,I2,M5,E1,P17);(A5,I2,M5,E1,P18);(A5,I2,M5,E1,P19);(A5,I2,M5,E1,P20);(A5,I2,M5,E2,P1);(A5,I2,M5,E2,P2);(A5,I2,M5,E2,P3);(A5,I2,M5,E2,P4);(A5,I2,M5,E2,P5);(A5,I2,M5,E2,P6);(A5,I2,M5,E2,P7);(A5,I2,M5,E2,P8);(A5,I2,M5,E2,P9);(A5,I2,M5,E2,P10);(A5,I2,M5,E2,P11);(A5,I2,M5,E2,P12);(A5,I2,M5,E2,P13);(A5,I2,M5,E2,P14);(A5,I2,M5,E2,P15);(A5,I2,M5,E2,P16);(A5,I2,M5,E2,P17);(A5,I2,M5,E2,P18);(A5,I2,M5,E2,P19);(A5,I2,M5,E2,P20);(A5,I2,M5,E3,P1);(A5,I2,M5,E3,P2);(A5,I2,M5,E3,P3);(A5,I2,M5,E3,P4);(A5,I2,M5,E3,P5);(A5,I2,M5,E3,P6);(A5,I2,M5,E3,P7);(A5,I2,M5,E3,P8);(A5,I2,M5,E3,P9);(A5,I2,M5,E3,P10);(A5,I2,M5,E3,P11);(A5,I2,M5,E3,P12);(A5,I2,M5,E3,P13);(A5,I2,M5,E3,P14);(A5,I2,M5,E3,P15);(A5,I2,M5,E3,P16);(A5,I2,M5,E3,P17);(A5,I2,M5,E3,P18);(A5,I2,M5,E3,P19);(A5,I2,M5,E3,P20);(A5,I2,M5,E4,P1);(A5,I2,M5,E4,P2);(A5,I2,M5,E4,P3);(A5,I2,M5,E4,P4);(A5,I2,M5,E4,P5);(A5,I2,M5,E4,P6);(A5,I2,M5,E4,P7);(A5,I2,M5,E4,P8);(A5,I2,M5,E4,P9);(A5,I2,M5,E4,P10);(A5,I2,M5,E4,P11);(A5,I2,M5,E4,P12);(A5,I2,M5,E4,P13);(A5,I2,M5,E4,P14);(A5,I2,M5,E4,P15);(A5,I2,M5,E4,P16);(A5,I2,M5,E4,P17);(A5,I2,M5,E4,P18);(A5,I2,M5,E4,P19);(A5,I2,M5,E4,P20);(A5,I2,M5,E5,P1);(A5,I2,M5,E5,P2);(A5,I2,M5,E5,P3);(A5,I2,M5,E5,P4);(A5,I2,M5,E5,P5);(A5,I2,M5,E5,P6);(A5,I2,M5,E5,P7);(A5,I2,M5,E5,P8);(A5,I2,M5,E5,P9);(A5,I2,M5,E5,P10);(A5,I2,M5,E5,P11);(A5,I2,M5,E5,P12);(A5,I2,M5,E5,P13);(A5,I2,M5,E5,P14);(A5,I2,M5,E5,P15);(A5,I2,M5,E5,P16);(A5,I2,M5,E5,P17);(A5,I2,M5,E5,P18);(A5,I2,M5,E5,P19);(A5,I2,M5,E5,P20);(A5,I2,M5,E6,P1);(A5,I2,M5,E6,P2);(A5,I2,M5,E6,P3);(A5,I2,M5,E6,P4);(A5,I2,M5,E6,P5);(A5,I2,M5,E6,P6);(A5,I2,M5,E6,P7);(A5,I2,M5,E6,P8);(A5,I2,M5,E6,P9);(A5,I2,M5,E6,P10);(A5,I2,M5,E6,P11);(A5,I2,M5,E6,P12);(A5,I2,M5,E6,P13);(A5,I2,M5,E6,P14);(A5,I2,M5,E6,P15);(A5,I2,M5,E6,P16);(A5,I2,M5,E6,P17);(A5,I2,M5,E6,P18);(A5,I2,M5,E6,P19);(A5,I2,M5,E6,P20);(A5,I2,M5,E7,P1);(A5,I2,M5,E7,P2);(A5,I2,M5,E7,P3);(A5,I2,M5,E7,P4);(A5,I2,M5,E7,P5);(A5,I2,M5,E7,P6);(A5,I2,M5,E7,P7);(A5,I2,M5,E7,P8);(A5,I2,M5,E7,P9);(A5,I2,M5,E7,P10);(A5,I2,M5,E7,P11);(A5,I2,M5,E7,P12);(A5,I2,M5,E7,P13);(A5,I2,M5,E7,P14);(A5,I2,M5,E7,P15);(A5,I2,M5,E7,P16);(A5,I2,M5,E7,P17);(A5,I2,M5,E7,P18);(A5,I2,M5,E7,P19);(A5,I2,M5,E7,P20);(A5,I2,M5,E8,P1);(A5,I2,M5,E8,P2);(A5,I2,M5,E8,P3);(A5,I2,M5,E8,P4);(A5,I2,M5,E8,P5);(A5,I2,M5,E8,P6);(A5,I2,M5,E8,P7);(A5,I2,M5,E8,P8);(A5,I2,M5,E8,P9);(A5,I2,M5,E8,P10);(A5,I2,M5,E8,P11);(A5,I2,M5,E8,P12);(A5,I2,M5,E8,P13);(A5,I2,M5,E8,P14);(A5,I2,M5,E8,P15);(A5,I2,M5,E8,P16);(A5,I2,M5,E8,P17);(A5,I2,M5,E8,P18);(A5,I2,M5,E8,P19);(A5,I2,M5,E8,P20);(A5,I2,M5,E9,P1);(A5,I2,M5,E9,P2);(A5,I2,M5,E9,P3);(A5,I2,M5,E9,P4);(A5,I2,M5,E9,P5);(A5,I2,M5,E9,P6);(A5,I2,M5,E9,P7);(A5,I2,M5,E9,P8);(A5,I2,M5,E9,P9);(A5,I2,M5,E9,P10);(A5,I2,M5,E9,P11);(A5,I2,M5,E9,P12);(A5,I2,M5,E9,P13);(A5,I2,M5,E9,P14);(A5,I2,M5,E9,P15);(A5,I2,M5,E9,P16);(A5,I2,M5,E9,P17);(A5,I2,M5,E9,P18);(A5,I2,M5,E9,P19);(A5,I2,M5,E9,P20);(A5,I2,M6,E1,P1);(A5,I2,M6,E1,P2);(A5,I2,M6,E1,P3);(A5,I2,M6,E1,P4);(A5,I2,M6,E1,P5);(A5,I2,M6,E1,P6);(A5,I2,M6,E1,P7);(A5,I2,M6,E1,P8);(A5,I2,M6,E1,P9);(A5,I2,M6,E1,P10);(A5,I2,M6,E1,P11);(A5,I2,M6,E1,P12);(A5,I2,M6,E1,P13);(A5,I2,M6,E1,P14);(A5,I2,M6,E1,P15);(A5,I2,M6,E1,P16);(A5,I2,M6,E1,P17);(A5,I2,M6,E1,P18);(A5,I2,M6,E1,P19);(A5,I2,M6,E1,P20);(A5,I2,M6,E2,P1);(A5,I2,M6,E2,P2);(A5,I2,M6,E2,P3);(A5,I2,M6,E2,P4);(A5,I2,M6,E2,P5);(A5,I2,M6,E2,P6);(A5,I2,M6,E2,P7);(A5,I2,M6,E2,P8);(A5,I2,M6,E2,P9);(A5,I2,M6,E2,P10);(A5,I2,M6,E2,P11);(A5,I2,M6,E2,P12);(A5,I2,M6,E2,P13);(A5,I2,M6,E2,P14);(A5,I2,M6,E2,P15);(A5,I2,M6,E2,P16);(A5,I2,M6,E2,P17);(A5,I2,M6,E2,P18);(A5,I2,M6,E2,P19);(A5,I2,M6,E2,P20);(A5,I2,M6,E3,P1);(A5,I2,M6,E3,P2);(A5,I2,M6,E3,P3);(A5,I2,M6,E3,P4);(A5,I2,M6,E3,P5);(A5,I2,M6,E3,P6);(A5,I2,M6,E3,P7);(A5,I2,M6,E3,P8);(A5,I2,M6,E3,P9);(A5,I2,M6,E3,P10);(A5,I2,M6,E3,P11);(A5,I2,M6,E3,P12);(A5,I2,M6,E3,P13);(A5,I2,M6,E3,P14);(A5,I2,M6,E3,P15);(A5,I2,M6,E3,P16);(A5,I2,M6,E3,P17);(A5,I2,M6,E3,P18);(A5,I2,M6,E3,P19);(A5,I2,M6,E3,P20);(A5,I2,M6,E4,P1);(A5,I2,M6,E4,P2);(A5,I2,M6,E4,P3);(A5,I2,M6,E4,P4);(A5,I2,M6,E4,P5);(A5,I2,M6,E4,P6);(A5,I2,M6,E4,P7);(A5,I2,M6,E4,P8);(A5,I2,M6,E4,P9);(A5,I2,M6,E4,P10);(A5,I2,M6,E4,P11);(A5,I2,M6,E4,P12);(A5,I2,M6,E4,P13);(A5,I2,M6,E4,P14);(A5,I2,M6,E4,P15);(A5,I2,M6,E4,P16);(A5,I2,M6,E4,P17);(A5,I2,M6,E4,P18);(A5,I2,M6,E4,P19);(A5,I2,M6,E4,P20);(A5,I2,M6,E5,P1);(A5,I2,M6,E5,P2);(A5,I2,M6,E5,P3);(A5,I2,M6,E5,P4);(A5,I2,M6,E5,P5);(A5,I2,M6,E5,P6);(A5,I2,M6,E5,P7);(A5,I2,M6,E5,P8);(A5,I2,M6,E5,P9);(A5,I2,M6,E5,P10);(A5,I2,M6,E5,P11);(A5,I2,M6,E5,P12);(A5,I2,M6,E5,P13);(A5,I2,M6,E5,P14);(A5,I2,M6,E5,P15);(A5,I2,M6,E5,P16);(A5,I2,M6,E5,P17);(A5,I2,M6,E5,P18);(A5,I2,M6,E5,P19);(A5,I2,M6,E5,P20);(A5,I2,M6,E6,P1);(A5,I2,M6,E6,P2);(A5,I2,M6,E6,P3);(A5,I2,M6,E6,P4);(A5,I2,M6,E6,P5);(A5,I2,M6,E6,P6);(A5,I2,M6,E6,P7);(A5,I2,M6,E6,P8);(A5,I2,M6,E6,P9);(A5,I2,M6,E6,P10);(A5,I2,M6,E6,P11);(A5,I2,M6,E6,P12);(A5,I2,M6,E6,P13);(A5,I2,M6,E6,P14);(A5,I2,M6,E6,P15);(A5,I2,M6,E6,P16);(A5,I2,M6,E6,P17);(A5,I2,M6,E6,P18);(A5,I2,M6,E6,P19);(A5,I2,M6,E6,P20);(A5,I2,M6,E7,P1);(A5,I2,M6,E7,P2);(A5,I2,M6,E7,P3);(A5,I2,M6,E7,P4);(A5,I2,M6,E7,P5);(A5,I2,M6,E7,P6);(A5,I2,M6,E7,P7);(A5,I2,M6,E7,P8);(A5,I2,M6,E7,P9);(A5,I2,M6,E7,P10);(A5,I2,M6,E7,P11);(A5,I2,M6,E7,P12);(A5,I2,M6,E7,P13);(A5,I2,M6,E7,P14);(A5,I2,M6,E7,P15);(A5,I2,M6,E7,P16);(A5,I2,M6,E7,P17);(A5,I2,M6,E7,P18);(A5,I2,M6,E7,P19);(A5,I2,M6,E7,P20);(A5,I2,M6,E8,P1);(A5,I2,M6,E8,P2);(A5,I2,M6,E8,P3);(A5,I2,M6,E8,P4);(A5,I2,M6,E8,P5);(A5,I2,M6,E8,P6);(A5,I2,M6,E8,P7);(A5,I2,M6,E8,P8);(A5,I2,M6,E8,P9);(A5,I2,M6,E8,P10);(A5,I2,M6,E8,P11);(A5,I2,M6,E8,P12);(A5,I2,M6,E8,P13);(A5,I2,M6,E8,P14);(A5,I2,M6,E8,P15);(A5,I2,M6,E8,P16);(A5,I2,M6,E8,P17);(A5,I2,M6,E8,P18);(A5,I2,M6,E8,P19);(A5,I2,M6,E8,P20);(A5,I2,M6,E9,P1);(A5,I2,M6,E9,P2);(A5,I2,M6,E9,P3);(A5,I2,M6,E9,P4);(A5,I2,M6,E9,P5);(A5,I2,M6,E9,P6);(A5,I2,M6,E9,P7);(A5,I2,M6,E9,P8);(A5,I2,M6,E9,P9);(A5,I2,M6,E9,P10);(A5,I2,M6,E9,P11);(A5,I2,M6,E9,P12);(A5,I2,M6,E9,P13);(A5,I2,M6,E9,P14);(A5,I2,M6,E9,P15);(A5,I2,M6,E9,P16);(A5,I2,M6,E9,P17);(A5,I2,M6,E9,P18);(A5,I2,M6,E9,P19);(A5,I2,M6,E9,P20);(A5,I3,M1,E1,P1);(A5,I3,M1,E1,P2);(A5,I3,M1,E1,P3);(A5,I3,M1,E1,P4);(A5,I3,M1,E1,P5);(A5,I3,M1,E1,P6);(A5,I3,M1,E1,P7);(A5,I3,M1,E1,P8);(A5,I3,M1,E1,P9);(A5,I3,M1,E1,P10);(A5,I3,M1,E1,P11);(A5,I3,M1,E1,P12);(A5,I3,M1,E1,P13);(A5,I3,M1,E1,P14);(A5,I3,M1,E1,P15);(A5,I3,M1,E1,P16);(A5,I3,M1,E1,P17);(A5,I3,M1,E1,P18);(A5,I3,M1,E1,P19);(A5,I3,M1,E1,P20);(A5,I3,M1,E2,P1);(A5,I3,M1,E2,P2);(A5,I3,M1,E2,P3);(A5,I3,M1,E2,P4);(A5,I3,M1,E2,P5);(A5,I3,M1,E2,P6);(A5,I3,M1,E2,P7);(A5,I3,M1,E2,P8);(A5,I3,M1,E2,P9);(A5,I3,M1,E2,P10);(A5,I3,M1,E2,P11);(A5,I3,M1,E2,P12);(A5,I3,M1,E2,P13);(A5,I3,M1,E2,P14);(A5,I3,M1,E2,P15);(A5,I3,M1,E2,P16);(A5,I3,M1,E2,P17);(A5,I3,M1,E2,P18);(A5,I3,M1,E2,P19);(A5,I3,M1,E2,P20);(A5,I3,M1,E3,P1);(A5,I3,M1,E3,P2);(A5,I3,M1,E3,P3);(A5,I3,M1,E3,P4);(A5,I3,M1,E3,P5);(A5,I3,M1,E3,P6);(A5,I3,M1,E3,P7);(A5,I3,M1,E3,P8);(A5,I3,M1,E3,P9);(A5,I3,M1,E3,P10);(A5,I3,M1,E3,P11);(A5,I3,M1,E3,P12);(A5,I3,M1,E3,P13);(A5,I3,M1,E3,P14);(A5,I3,M1,E3,P15);(A5,I3,M1,E3,P16);(A5,I3,M1,E3,P17);(A5,I3,M1,E3,P18);(A5,I3,M1,E3,P19);(A5,I3,M1,E3,P20);(A5,I3,M1,E4,P1);(A5,I3,M1,E4,P2);(A5,I3,M1,E4,P3);(A5,I3,M1,E4,P4);(A5,I3,M1,E4,P5);(A5,I3,M1,E4,P6);(A5,I3,M1,E4,P7);(A5,I3,M1,E4,P8);(A5,I3,M1,E4,P9);(A5,I3,M1,E4,P10);(A5,I3,M1,E4,P11);(A5,I3,M1,E4,P12);(A5,I3,M1,E4,P13);(A5,I3,M1,E4,P14);(A5,I3,M1,E4,P15);(A5,I3,M1,E4,P16);(A5,I3,M1,E4,P17);(A5,I3,M1,E4,P18);(A5,I3,M1,E4,P19);(A5,I3,M1,E4,P20);(A5,I3,M1,E5,P1);(A5,I3,M1,E5,P2);(A5,I3,M1,E5,P3);(A5,I3,M1,E5,P4);(A5,I3,M1,E5,P5);(A5,I3,M1,E5,P6);(A5,I3,M1,E5,P7);(A5,I3,M1,E5,P8);(A5,I3,M1,E5,P9);(A5,I3,M1,E5,P10);(A5,I3,M1,E5,P11);(A5,I3,M1,E5,P12);(A5,I3,M1,E5,P13);(A5,I3,M1,E5,P14);(A5,I3,M1,E5,P15);(A5,I3,M1,E5,P16);(A5,I3,M1,E5,P17);(A5,I3,M1,E5,P18);(A5,I3,M1,E5,P19);(A5,I3,M1,E5,P20);(A5,I3,M1,E6,P1);(A5,I3,M1,E6,P2);(A5,I3,M1,E6,P3);(A5,I3,M1,E6,P4);(A5,I3,M1,E6,P5);(A5,I3,M1,E6,P6);(A5,I3,M1,E6,P7);(A5,I3,M1,E6,P8);(A5,I3,M1,E6,P9);(A5,I3,M1,E6,P10);(A5,I3,M1,E6,P11);(A5,I3,M1,E6,P12);(A5,I3,M1,E6,P13);(A5,I3,M1,E6,P14);(A5,I3,M1,E6,P15);(A5,I3,M1,E6,P16);(A5,I3,M1,E6,P17);(A5,I3,M1,E6,P18);(A5,I3,M1,E6,P19);(A5,I3,M1,E6,P20);(A5,I3,M1,E7,P1);(A5,I3,M1,E7,P2);(A5,I3,M1,E7,P3);(A5,I3,M1,E7,P4);(A5,I3,M1,E7,P5);(A5,I3,M1,E7,P6);(A5,I3,M1,E7,P7);(A5,I3,M1,E7,P8);(A5,I3,M1,E7,P9);(A5,I3,M1,E7,P10);(A5,I3,M1,E7,P11);(A5,I3,M1,E7,P12);(A5,I3,M1,E7,P13);(A5,I3,M1,E7,P14);(A5,I3,M1,E7,P15);(A5,I3,M1,E7,P16);(A5,I3,M1,E7,P17);(A5,I3,M1,E7,P18);(A5,I3,M1,E7,P19);(A5,I3,M1,E7,P20);(A5,I3,M1,E8,P1);(A5,I3,M1,E8,P2);(A5,I3,M1,E8,P3);(A5,I3,M1,E8,P4);(A5,I3,M1,E8,P5);(A5,I3,M1,E8,P6);(A5,I3,M1,E8,P7);(A5,I3,M1,E8,P8);(A5,I3,M1,E8,P9);(A5,I3,M1,E8,P10);(A5,I3,M1,E8,P11);(A5,I3,M1,E8,P12);(A5,I3,M1,E8,P13);(A5,I3,M1,E8,P14);(A5,I3,M1,E8,P15);(A5,I3,M1,E8,P16);(A5,I3,M1,E8,P17);(A5,I3,M1,E8,P18);(A5,I3,M1,E8,P19);(A5,I3,M1,E8,P20);(A5,I3,M1,E9,P1);(A5,I3,M1,E9,P2);(A5,I3,M1,E9,P3);(A5,I3,M1,E9,P4);(A5,I3,M1,E9,P5);(A5,I3,M1,E9,P6);(A5,I3,M1,E9,P7);(A5,I3,M1,E9,P8);(A5,I3,M1,E9,P9);(A5,I3,M1,E9,P10);(A5,I3,M1,E9,P11);(A5,I3,M1,E9,P12);(A5,I3,M1,E9,P13);(A5,I3,M1,E9,P14);(A5,I3,M1,E9,P15);(A5,I3,M1,E9,P16);(A5,I3,M1,E9,P17);(A5,I3,M1,E9,P18);(A5,I3,M1,E9,P19);(A5,I3,M1,E9,P20);(A5,I3,M2,E1,P1);(A5,I3,M2,E1,P2);(A5,I3,M2,E1,P3);(A5,I3,M2,E1,P4);(A5,I3,M2,E1,P5);(A5,I3,M2,E1,P6);(A5,I3,M2,E1,P7);(A5,I3,M2,E1,P8);(A5,I3,M2,E1,P9);(A5,I3,M2,E1,P10);(A5,I3,M2,E1,P11);(A5,I3,M2,E1,P12);(A5,I3,M2,E1,P13);(A5,I3,M2,E1,P14);(A5,I3,M2,E1,P15);(A5,I3,M2,E1,P16);(A5,I3,M2,E1,P17);(A5,I3,M2,E1,P18);(A5,I3,M2,E1,P19);(A5,I3,M2,E1,P20);(A5,I3,M2,E2,P1);(A5,I3,M2,E2,P2);(A5,I3,M2,E2,P3);(A5,I3,M2,E2,P4);(A5,I3,M2,E2,P5);(A5,I3,M2,E2,P6);(A5,I3,M2,E2,P7);(A5,I3,M2,E2,P8);(A5,I3,M2,E2,P9);(A5,I3,M2,E2,P10);(A5,I3,M2,E2,P11);(A5,I3,M2,E2,P12);(A5,I3,M2,E2,P13);(A5,I3,M2,E2,P14);(A5,I3,M2,E2,P15);(A5,I3,M2,E2,P16);(A5,I3,M2,E2,P17);(A5,I3,M2,E2,P18);(A5,I3,M2,E2,P19);(A5,I3,M2,E2,P20);(A5,I3,M2,E3,P1);(A5,I3,M2,E3,P2);(A5,I3,M2,E3,P3);(A5,I3,M2,E3,P4);(A5,I3,M2,E3,P5);(A5,I3,M2,E3,P6);(A5,I3,M2,E3,P7);(A5,I3,M2,E3,P8);(A5,I3,M2,E3,P9);(A5,I3,M2,E3,P10);(A5,I3,M2,E3,P11);(A5,I3,M2,E3,P12);(A5,I3,M2,E3,P13);(A5,I3,M2,E3,P14);(A5,I3,M2,E3,P15);(A5,I3,M2,E3,P16);(A5,I3,M2,E3,P17);(A5,I3,M2,E3,P18);(A5,I3,M2,E3,P19);(A5,I3,M2,E3,P20);(A5,I3,M2,E4,P1);(A5,I3,M2,E4,P2);(A5,I3,M2,E4,P3);(A5,I3,M2,E4,P4);(A5,I3,M2,E4,P5);(A5,I3,M2,E4,P6);(A5,I3,M2,E4,P7);(A5,I3,M2,E4,P8);(A5,I3,M2,E4,P9);(A5,I3,M2,E4,P10);(A5,I3,M2,E4,P11);(A5,I3,M2,E4,P12);(A5,I3,M2,E4,P13);(A5,I3,M2,E4,P14);(A5,I3,M2,E4,P15);(A5,I3,M2,E4,P16);(A5,I3,M2,E4,P17);(A5,I3,M2,E4,P18);(A5,I3,M2,E4,P19);(A5,I3,M2,E4,P20);(A5,I3,M2,E5,P1);(A5,I3,M2,E5,P2);(A5,I3,M2,E5,P3);(A5,I3,M2,E5,P4);(A5,I3,M2,E5,P5);(A5,I3,M2,E5,P6);(A5,I3,M2,E5,P7);(A5,I3,M2,E5,P8);(A5,I3,M2,E5,P9);(A5,I3,M2,E5,P10);(A5,I3,M2,E5,P11);(A5,I3,M2,E5,P12);(A5,I3,M2,E5,P13);(A5,I3,M2,E5,P14);(A5,I3,M2,E5,P15);(A5,I3,M2,E5,P16);(A5,I3,M2,E5,P17);(A5,I3,M2,E5,P18);(A5,I3,M2,E5,P19);(A5,I3,M2,E5,P20);(A5,I3,M2,E6,P1);(A5,I3,M2,E6,P2);(A5,I3,M2,E6,P3);(A5,I3,M2,E6,P4);(A5,I3,M2,E6,P5);(A5,I3,M2,E6,P6);(A5,I3,M2,E6,P7);(A5,I3,M2,E6,P8);(A5,I3,M2,E6,P9);(A5,I3,M2,E6,P10);(A5,I3,M2,E6,P11);(A5,I3,M2,E6,P12);(A5,I3,M2,E6,P13);(A5,I3,M2,E6,P14);(A5,I3,M2,E6,P15);(A5,I3,M2,E6,P16);(A5,I3,M2,E6,P17);(A5,I3,M2,E6,P18);(A5,I3,M2,E6,P19);(A5,I3,M2,E6,P20);(A5,I3,M2,E7,P1);(A5,I3,M2,E7,P2);(A5,I3,M2,E7,P3);(A5,I3,M2,E7,P4);(A5,I3,M2,E7,P5);(A5,I3,M2,E7,P6);(A5,I3,M2,E7,P7);(A5,I3,M2,E7,P8);(A5,I3,M2,E7,P9);(A5,I3,M2,E7,P10);(A5,I3,M2,E7,P11);(A5,I3,M2,E7,P12);(A5,I3,M2,E7,P13);(A5,I3,M2,E7,P14);(A5,I3,M2,E7,P15);(A5,I3,M2,E7,P16);(A5,I3,M2,E7,P17);(A5,I3,M2,E7,P18);(A5,I3,M2,E7,P19);(A5,I3,M2,E7,P20);(A5,I3,M2,E8,P1);(A5,I3,M2,E8,P2);(A5,I3,M2,E8,P3);(A5,I3,M2,E8,P4);(A5,I3,M2,E8,P5);(A5,I3,M2,E8,P6);(A5,I3,M2,E8,P7);(A5,I3,M2,E8,P8);(A5,I3,M2,E8,P9);(A5,I3,M2,E8,P10);(A5,I3,M2,E8,P11);(A5,I3,M2,E8,P12);(A5,I3,M2,E8,P13);(A5,I3,M2,E8,P14);(A5,I3,M2,E8,P15);(A5,I3,M2,E8,P16);(A5,I3,M2,E8,P17);(A5,I3,M2,E8,P18);(A5,I3,M2,E8,P19);(A5,I3,M2,E8,P20);(A5,I3,M2,E9,P1);(A5,I3,M2,E9,P2);(A5,I3,M2,E9,P3);(A5,I3,M2,E9,P4);(A5,I3,M2,E9,P5);(A5,I3,M2,E9,P6);(A5,I3,M2,E9,P7);(A5,I3,M2,E9,P8);(A5,I3,M2,E9,P9);(A5,I3,M2,E9,P10);(A5,I3,M2,E9,P11);(A5,I3,M2,E9,P12);(A5,I3,M2,E9,P13);(A5,I3,M2,E9,P14);(A5,I3,M2,E9,P15);(A5,I3,M2,E9,P16);(A5,I3,M2,E9,P17);(A5,I3,M2,E9,P18);(A5,I3,M2,E9,P19);(A5,I3,M2,E9,P20);(A5,I3,M3,E1,P1);(A5,I3,M3,E1,P2);(A5,I3,M3,E1,P3);(A5,I3,M3,E1,P4);(A5,I3,M3,E1,P5);(A5,I3,M3,E1,P6);(A5,I3,M3,E1,P7);(A5,I3,M3,E1,P8);(A5,I3,M3,E1,P9);(A5,I3,M3,E1,P10);(A5,I3,M3,E1,P11);(A5,I3,M3,E1,P12);(A5,I3,M3,E1,P13);(A5,I3,M3,E1,P14);(A5,I3,M3,E1,P15);(A5,I3,M3,E1,P16);(A5,I3,M3,E1,P17);(A5,I3,M3,E1,P18);(A5,I3,M3,E1,P19);(A5,I3,M3,E1,P20);(A5,I3,M3,E2,P1);(A5,I3,M3,E2,P2);(A5,I3,M3,E2,P3);(A5,I3,M3,E2,P4);(A5,I3,M3,E2,P5);(A5,I3,M3,E2,P6);(A5,I3,M3,E2,P7);(A5,I3,M3,E2,P8);(A5,I3,M3,E2,P9);(A5,I3,M3,E2,P10);(A5,I3,M3,E2,P11);(A5,I3,M3,E2,P12);(A5,I3,M3,E2,P13);(A5,I3,M3,E2,P14);(A5,I3,M3,E2,P15);(A5,I3,M3,E2,P16);(A5,I3,M3,E2,P17);(A5,I3,M3,E2,P18);(A5,I3,M3,E2,P19);(A5,I3,M3,E2,P20);(A5,I3,M3,E3,P1);(A5,I3,M3,E3,P2);(A5,I3,M3,E3,P3);(A5,I3,M3,E3,P4);(A5,I3,M3,E3,P5);(A5,I3,M3,E3,P6);(A5,I3,M3,E3,P7);(A5,I3,M3,E3,P8);(A5,I3,M3,E3,P9);(A5,I3,M3,E3,P10);(A5,I3,M3,E3,P11);(A5,I3,M3,E3,P12);(A5,I3,M3,E3,P13);(A5,I3,M3,E3,P14);(A5,I3,M3,E3,P15);(A5,I3,M3,E3,P16);(A5,I3,M3,E3,P17);(A5,I3,M3,E3,P18);(A5,I3,M3,E3,P19);(A5,I3,M3,E3,P20);(A5,I3,M3,E4,P1);(A5,I3,M3,E4,P2);(A5,I3,M3,E4,P3);(A5,I3,M3,E4,P4);(A5,I3,M3,E4,P5);(A5,I3,M3,E4,P6);(A5,I3,M3,E4,P7);(A5,I3,M3,E4,P8);(A5,I3,M3,E4,P9);(A5,I3,M3,E4,P10);(A5,I3,M3,E4,P11);(A5,I3,M3,E4,P12);(A5,I3,M3,E4,P13);(A5,I3,M3,E4,P14);(A5,I3,M3,E4,P15);(A5,I3,M3,E4,P16);(A5,I3,M3,E4,P17);(A5,I3,M3,E4,P18);(A5,I3,M3,E4,P19);(A5,I3,M3,E4,P20);(A5,I3,M3,E5,P1);(A5,I3,M3,E5,P2);(A5,I3,M3,E5,P3);(A5,I3,M3,E5,P4);(A5,I3,M3,E5,P5);(A5,I3,M3,E5,P6);(A5,I3,M3,E5,P7);(A5,I3,M3,E5,P8);(A5,I3,M3,E5,P9);(A5,I3,M3,E5,P10);(A5,I3,M3,E5,P11);(A5,I3,M3,E5,P12);(A5,I3,M3,E5,P13);(A5,I3,M3,E5,P14);(A5,I3,M3,E5,P15);(A5,I3,M3,E5,P16);(A5,I3,M3,E5,P17);(A5,I3,M3,E5,P18);(A5,I3,M3,E5,P19);(A5,I3,M3,E5,P20);(A5,I3,M3,E6,P1);(A5,I3,M3,E6,P2);(A5,I3,M3,E6,P3);(A5,I3,M3,E6,P4);(A5,I3,M3,E6,P5);(A5,I3,M3,E6,P6);(A5,I3,M3,E6,P7);(A5,I3,M3,E6,P8);(A5,I3,M3,E6,P9);(A5,I3,M3,E6,P10);(A5,I3,M3,E6,P11);(A5,I3,M3,E6,P12);(A5,I3,M3,E6,P13);(A5,I3,M3,E6,P14);(A5,I3,M3,E6,P15);(A5,I3,M3,E6,P16);(A5,I3,M3,E6,P17);(A5,I3,M3,E6,P18);(A5,I3,M3,E6,P19);(A5,I3,M3,E6,P20);(A5,I3,M3,E7,P1);(A5,I3,M3,E7,P2);(A5,I3,M3,E7,P3);(A5,I3,M3,E7,P4);(A5,I3,M3,E7,P5);(A5,I3,M3,E7,P6);(A5,I3,M3,E7,P7);(A5,I3,M3,E7,P8);(A5,I3,M3,E7,P9);(A5,I3,M3,E7,P10);(A5,I3,M3,E7,P11);(A5,I3,M3,E7,P12);(A5,I3,M3,E7,P13);(A5,I3,M3,E7,P14);(A5,I3,M3,E7,P15);(A5,I3,M3,E7,P16);(A5,I3,M3,E7,P17);(A5,I3,M3,E7,P18);(A5,I3,M3,E7,P19);(A5,I3,M3,E7,P20);(A5,I3,M3,E8,P1);(A5,I3,M3,E8,P2);(A5,I3,M3,E8,P3);(A5,I3,M3,E8,P4);(A5,I3,M3,E8,P5);(A5,I3,M3,E8,P6);(A5,I3,M3,E8,P7);(A5,I3,M3,E8,P8);(A5,I3,M3,E8,P9);(A5,I3,M3,E8,P10);(A5,I3,M3,E8,P11);(A5,I3,M3,E8,P12);(A5,I3,M3,E8,P13);(A5,I3,M3,E8,P14);(A5,I3,M3,E8,P15);(A5,I3,M3,E8,P16);(A5,I3,M3,E8,P17);(A5,I3,M3,E8,P18);(A5,I3,M3,E8,P19);(A5,I3,M3,E8,P20);(A5,I3,M3,E9,P1);(A5,I3,M3,E9,P2);(A5,I3,M3,E9,P3);(A5,I3,M3,E9,P4);(A5,I3,M3,E9,P5);(A5,I3,M3,E9,P6);(A5,I3,M3,E9,P7);(A5,I3,M3,E9,P8);(A5,I3,M3,E9,P9);(A5,I3,M3,E9,P10);(A5,I3,M3,E9,P11);(A5,I3,M3,E9,P12);(A5,I3,M3,E9,P13);(A5,I3,M3,E9,P14);(A5,I3,M3,E9,P15);(A5,I3,M3,E9,P16);(A5,I3,M3,E9,P17);(A5,I3,M3,E9,P18);(A5,I3,M3,E9,P19);(A5,I3,M3,E9,P20);(A5,I3,M4,E1,P1);(A5,I3,M4,E1,P2);(A5,I3,M4,E1,P3);(A5,I3,M4,E1,P4);(A5,I3,M4,E1,P5);(A5,I3,M4,E1,P6);(A5,I3,M4,E1,P7);(A5,I3,M4,E1,P8);(A5,I3,M4,E1,P9);(A5,I3,M4,E1,P10);(A5,I3,M4,E1,P11);(A5,I3,M4,E1,P12);(A5,I3,M4,E1,P13);(A5,I3,M4,E1,P14);(A5,I3,M4,E1,P15);(A5,I3,M4,E1,P16);(A5,I3,M4,E1,P17);(A5,I3,M4,E1,P18);(A5,I3,M4,E1,P19);(A5,I3,M4,E1,P20);(A5,I3,M4,E2,P1);(A5,I3,M4,E2,P2);(A5,I3,M4,E2,P3);(A5,I3,M4,E2,P4);(A5,I3,M4,E2,P5);(A5,I3,M4,E2,P6);(A5,I3,M4,E2,P7);(A5,I3,M4,E2,P8);(A5,I3,M4,E2,P9);(A5,I3,M4,E2,P10);(A5,I3,M4,E2,P11);(A5,I3,M4,E2,P12);(A5,I3,M4,E2,P13);(A5,I3,M4,E2,P14);(A5,I3,M4,E2,P15);(A5,I3,M4,E2,P16);(A5,I3,M4,E2,P17);(A5,I3,M4,E2,P18);(A5,I3,M4,E2,P19);(A5,I3,M4,E2,P20);(A5,I3,M4,E3,P1);(A5,I3,M4,E3,P2);(A5,I3,M4,E3,P3);(A5,I3,M4,E3,P4);(A5,I3,M4,E3,P5);(A5,I3,M4,E3,P6);(A5,I3,M4,E3,P7);(A5,I3,M4,E3,P8);(A5,I3,M4,E3,P9);(A5,I3,M4,E3,P10);(A5,I3,M4,E3,P11);(A5,I3,M4,E3,P12);(A5,I3,M4,E3,P13);(A5,I3,M4,E3,P14);(A5,I3,M4,E3,P15);(A5,I3,M4,E3,P16);(A5,I3,M4,E3,P17);(A5,I3,M4,E3,P18);(A5,I3,M4,E3,P19);(A5,I3,M4,E3,P20);(A5,I3,M4,E4,P1);(A5,I3,M4,E4,P2);(A5,I3,M4,E4,P3);(A5,I3,M4,E4,P4);(A5,I3,M4,E4,P5);(A5,I3,M4,E4,P6);(A5,I3,M4,E4,P7);(A5,I3,M4,E4,P8);(A5,I3,M4,E4,P9);(A5,I3,M4,E4,P10);(A5,I3,M4,E4,P11);(A5,I3,M4,E4,P12);(A5,I3,M4,E4,P13);(A5,I3,M4,E4,P14);(A5,I3,M4,E4,P15);(A5,I3,M4,E4,P16);(A5,I3,M4,E4,P17);(A5,I3,M4,E4,P18);(A5,I3,M4,E4,P19);(A5,I3,M4,E4,P20);(A5,I3,M4,E5,P1);(A5,I3,M4,E5,P2);(A5,I3,M4,E5,P3);(A5,I3,M4,E5,P4);(A5,I3,M4,E5,P5);(A5,I3,M4,E5,P6);(A5,I3,M4,E5,P7);(A5,I3,M4,E5,P8);(A5,I3,M4,E5,P9);(A5,I3,M4,E5,P10);(A5,I3,M4,E5,P11);(A5,I3,M4,E5,P12);(A5,I3,M4,E5,P13);(A5,I3,M4,E5,P14);(A5,I3,M4,E5,P15);(A5,I3,M4,E5,P16);(A5,I3,M4,E5,P17);(A5,I3,M4,E5,P18);(A5,I3,M4,E5,P19);(A5,I3,M4,E5,P20);(A5,I3,M4,E6,P1);(A5,I3,M4,E6,P2);(A5,I3,M4,E6,P3);(A5,I3,M4,E6,P4);(A5,I3,M4,E6,P5);(A5,I3,M4,E6,P6);(A5,I3,M4,E6,P7);(A5,I3,M4,E6,P8);(A5,I3,M4,E6,P9);(A5,I3,M4,E6,P10);(A5,I3,M4,E6,P11);(A5,I3,M4,E6,P12);(A5,I3,M4,E6,P13);(A5,I3,M4,E6,P14);(A5,I3,M4,E6,P15);(A5,I3,M4,E6,P16);(A5,I3,M4,E6,P17);(A5,I3,M4,E6,P18);(A5,I3,M4,E6,P19);(A5,I3,M4,E6,P20);(A5,I3,M4,E7,P1);(A5,I3,M4,E7,P2);(A5,I3,M4,E7,P3);(A5,I3,M4,E7,P4);(A5,I3,M4,E7,P5);(A5,I3,M4,E7,P6);(A5,I3,M4,E7,P7);(A5,I3,M4,E7,P8);(A5,I3,M4,E7,P9);(A5,I3,M4,E7,P10);(A5,I3,M4,E7,P11);(A5,I3,M4,E7,P12);(A5,I3,M4,E7,P13);(A5,I3,M4,E7,P14);(A5,I3,M4,E7,P15);(A5,I3,M4,E7,P16);(A5,I3,M4,E7,P17);(A5,I3,M4,E7,P18);(A5,I3,M4,E7,P19);(A5,I3,M4,E7,P20);(A5,I3,M4,E8,P1);(A5,I3,M4,E8,P2);(A5,I3,M4,E8,P3);(A5,I3,M4,E8,P4);(A5,I3,M4,E8,P5);(A5,I3,M4,E8,P6);(A5,I3,M4,E8,P7);(A5,I3,M4,E8,P8);(A5,I3,M4,E8,P9);(A5,I3,M4,E8,P10);(A5,I3,M4,E8,P11);(A5,I3,M4,E8,P12);(A5,I3,M4,E8,P13);(A5,I3,M4,E8,P14);(A5,I3,M4,E8,P15);(A5,I3,M4,E8,P16);(A5,I3,M4,E8,P17);(A5,I3,M4,E8,P18);(A5,I3,M4,E8,P19);(A5,I3,M4,E8,P20);(A5,I3,M4,E9,P1);(A5,I3,M4,E9,P2);(A5,I3,M4,E9,P3);(A5,I3,M4,E9,P4);(A5,I3,M4,E9,P5);(A5,I3,M4,E9,P6);(A5,I3,M4,E9,P7);(A5,I3,M4,E9,P8);(A5,I3,M4,E9,P9);(A5,I3,M4,E9,P10);(A5,I3,M4,E9,P11);(A5,I3,M4,E9,P12);(A5,I3,M4,E9,P13);(A5,I3,M4,E9,P14);(A5,I3,M4,E9,P15);(A5,I3,M4,E9,P16);(A5,I3,M4,E9,P17);(A5,I3,M4,E9,P18);(A5,I3,M4,E9,P19);(A5,I3,M4,E9,P20);(A5,I3,M5,E1,P1);(A5,I3,M5,E1,P2);(A5,I3,M5,E1,P3);(A5,I3,M5,E1,P4);(A5,I3,M5,E1,P5);(A5,I3,M5,E1,P6);(A5,I3,M5,E1,P7);(A5,I3,M5,E1,P8);(A5,I3,M5,E1,P9);(A5,I3,M5,E1,P10);(A5,I3,M5,E1,P11);(A5,I3,M5,E1,P12);(A5,I3,M5,E1,P13);(A5,I3,M5,E1,P14);(A5,I3,M5,E1,P15);(A5,I3,M5,E1,P16);(A5,I3,M5,E1,P17);(A5,I3,M5,E1,P18);(A5,I3,M5,E1,P19);(A5,I3,M5,E1,P20);(A5,I3,M5,E2,P1);(A5,I3,M5,E2,P2);(A5,I3,M5,E2,P3);(A5,I3,M5,E2,P4);(A5,I3,M5,E2,P5);(A5,I3,M5,E2,P6);(A5,I3,M5,E2,P7);(A5,I3,M5,E2,P8);(A5,I3,M5,E2,P9);(A5,I3,M5,E2,P10);(A5,I3,M5,E2,P11);(A5,I3,M5,E2,P12);(A5,I3,M5,E2,P13);(A5,I3,M5,E2,P14);(A5,I3,M5,E2,P15);(A5,I3,M5,E2,P16);(A5,I3,M5,E2,P17);(A5,I3,M5,E2,P18);(A5,I3,M5,E2,P19);(A5,I3,M5,E2,P20);(A5,I3,M5,E3,P1);(A5,I3,M5,E3,P2);(A5,I3,M5,E3,P3);(A5,I3,M5,E3,P4);(A5,I3,M5,E3,P5);(A5,I3,M5,E3,P6);(A5,I3,M5,E3,P7);(A5,I3,M5,E3,P8);(A5,I3,M5,E3,P9);(A5,I3,M5,E3,P10);(A5,I3,M5,E3,P11);(A5,I3,M5,E3,P12);(A5,I3,M5,E3,P13);(A5,I3,M5,E3,P14);(A5,I3,M5,E3,P15);(A5,I3,M5,E3,P16);(A5,I3,M5,E3,P17);(A5,I3,M5,E3,P18);(A5,I3,M5,E3,P19);(A5,I3,M5,E3,P20);(A5,I3,M5,E4,P1);(A5,I3,M5,E4,P2);(A5,I3,M5,E4,P3);(A5,I3,M5,E4,P4);(A5,I3,M5,E4,P5);(A5,I3,M5,E4,P6);(A5,I3,M5,E4,P7);(A5,I3,M5,E4,P8);(A5,I3,M5,E4,P9);(A5,I3,M5,E4,P10);(A5,I3,M5,E4,P11);(A5,I3,M5,E4,P12);(A5,I3,M5,E4,P13);(A5,I3,M5,E4,P14);(A5,I3,M5,E4,P15);(A5,I3,M5,E4,P16);(A5,I3,M5,E4,P17);(A5,I3,M5,E4,P18);(A5,I3,M5,E4,P19);(A5,I3,M5,E4,P20);(A5,I3,M5,E5,P1);(A5,I3,M5,E5,P2);(A5,I3,M5,E5,P3);(A5,I3,M5,E5,P4);(A5,I3,M5,E5,P5);(A5,I3,M5,E5,P6);(A5,I3,M5,E5,P7);(A5,I3,M5,E5,P8);(A5,I3,M5,E5,P9);(A5,I3,M5,E5,P10);(A5,I3,M5,E5,P11);(A5,I3,M5,E5,P12);(A5,I3,M5,E5,P13);(A5,I3,M5,E5,P14);(A5,I3,M5,E5,P15);(A5,I3,M5,E5,P16);(A5,I3,M5,E5,P17);(A5,I3,M5,E5,P18);(A5,I3,M5,E5,P19);(A5,I3,M5,E5,P20);(A5,I3,M5,E6,P1);(A5,I3,M5,E6,P2);(A5,I3,M5,E6,P3);(A5,I3,M5,E6,P4);(A5,I3,M5,E6,P5);(A5,I3,M5,E6,P6);(A5,I3,M5,E6,P7);(A5,I3,M5,E6,P8);(A5,I3,M5,E6,P9);(A5,I3,M5,E6,P10);(A5,I3,M5,E6,P11);(A5,I3,M5,E6,P12);(A5,I3,M5,E6,P13);(A5,I3,M5,E6,P14);(A5,I3,M5,E6,P15);(A5,I3,M5,E6,P16);(A5,I3,M5,E6,P17);(A5,I3,M5,E6,P18);(A5,I3,M5,E6,P19);(A5,I3,M5,E6,P20);(A5,I3,M5,E7,P1);(A5,I3,M5,E7,P2);(A5,I3,M5,E7,P3);(A5,I3,M5,E7,P4);(A5,I3,M5,E7,P5);(A5,I3,M5,E7,P6);(A5,I3,M5,E7,P7);(A5,I3,M5,E7,P8);(A5,I3,M5,E7,P9);(A5,I3,M5,E7,P10);(A5,I3,M5,E7,P11);(A5,I3,M5,E7,P12);(A5,I3,M5,E7,P13);(A5,I3,M5,E7,P14);(A5,I3,M5,E7,P15);(A5,I3,M5,E7,P16);(A5,I3,M5,E7,P17);(A5,I3,M5,E7,P18);(A5,I3,M5,E7,P19);(A5,I3,M5,E7,P20);(A5,I3,M5,E8,P1);(A5,I3,M5,E8,P2);(A5,I3,M5,E8,P3);(A5,I3,M5,E8,P4);(A5,I3,M5,E8,P5);(A5,I3,M5,E8,P6);(A5,I3,M5,E8,P7);(A5,I3,M5,E8,P8);(A5,I3,M5,E8,P9);(A5,I3,M5,E8,P10);(A5,I3,M5,E8,P11);(A5,I3,M5,E8,P12);(A5,I3,M5,E8,P13);(A5,I3,M5,E8,P14);(A5,I3,M5,E8,P15);(A5,I3,M5,E8,P16);(A5,I3,M5,E8,P17);(A5,I3,M5,E8,P18);(A5,I3,M5,E8,P19);(A5,I3,M5,E8,P20);(A5,I3,M5,E9,P1);(A5,I3,M5,E9,P2);(A5,I3,M5,E9,P3);(A5,I3,M5,E9,P4);(A5,I3,M5,E9,P5);(A5,I3,M5,E9,P6);(A5,I3,M5,E9,P7);(A5,I3,M5,E9,P8);(A5,I3,M5,E9,P9);(A5,I3,M5,E9,P10);(A5,I3,M5,E9,P11);(A5,I3,M5,E9,P12);(A5,I3,M5,E9,P13);(A5,I3,M5,E9,P14);(A5,I3,M5,E9,P15);(A5,I3,M5,E9,P16);(A5,I3,M5,E9,P17);(A5,I3,M5,E9,P18);(A5,I3,M5,E9,P19);(A5,I3,M5,E9,P20);(A5,I3,M6,E1,P1);(A5,I3,M6,E1,P2);(A5,I3,M6,E1,P3);(A5,I3,M6,E1,P4);(A5,I3,M6,E1,P5);(A5,I3,M6,E1,P6);(A5,I3,M6,E1,P7);(A5,I3,M6,E1,P8);(A5,I3,M6,E1,P9);(A5,I3,M6,E1,P10);(A5,I3,M6,E1,P11);(A5,I3,M6,E1,P12);(A5,I3,M6,E1,P13);(A5,I3,M6,E1,P14);(A5,I3,M6,E1,P15);(A5,I3,M6,E1,P16);(A5,I3,M6,E1,P17);(A5,I3,M6,E1,P18);(A5,I3,M6,E1,P19);(A5,I3,M6,E1,P20);(A5,I3,M6,E2,P1);(A5,I3,M6,E2,P2);(A5,I3,M6,E2,P3);(A5,I3,M6,E2,P4);(A5,I3,M6,E2,P5);(A5,I3,M6,E2,P6);(A5,I3,M6,E2,P7);(A5,I3,M6,E2,P8);(A5,I3,M6,E2,P9);(A5,I3,M6,E2,P10);(A5,I3,M6,E2,P11);(A5,I3,M6,E2,P12);(A5,I3,M6,E2,P13);(A5,I3,M6,E2,P14);(A5,I3,M6,E2,P15);(A5,I3,M6,E2,P16);(A5,I3,M6,E2,P17);(A5,I3,M6,E2,P18);(A5,I3,M6,E2,P19);(A5,I3,M6,E2,P20);(A5,I3,M6,E3,P1);(A5,I3,M6,E3,P2);(A5,I3,M6,E3,P3);(A5,I3,M6,E3,P4);(A5,I3,M6,E3,P5);(A5,I3,M6,E3,P6);(A5,I3,M6,E3,P7);(A5,I3,M6,E3,P8);(A5,I3,M6,E3,P9);(A5,I3,M6,E3,P10);(A5,I3,M6,E3,P11);(A5,I3,M6,E3,P12);(A5,I3,M6,E3,P13);(A5,I3,M6,E3,P14);(A5,I3,M6,E3,P15);(A5,I3,M6,E3,P16);(A5,I3,M6,E3,P17);(A5,I3,M6,E3,P18);(A5,I3,M6,E3,P19);(A5,I3,M6,E3,P20);(A5,I3,M6,E4,P1);(A5,I3,M6,E4,P2);(A5,I3,M6,E4,P3);(A5,I3,M6,E4,P4);(A5,I3,M6,E4,P5);(A5,I3,M6,E4,P6);(A5,I3,M6,E4,P7);(A5,I3,M6,E4,P8);(A5,I3,M6,E4,P9);(A5,I3,M6,E4,P10);(A5,I3,M6,E4,P11);(A5,I3,M6,E4,P12);(A5,I3,M6,E4,P13);(A5,I3,M6,E4,P14);(A5,I3,M6,E4,P15);(A5,I3,M6,E4,P16);(A5,I3,M6,E4,P17);(A5,I3,M6,E4,P18);(A5,I3,M6,E4,P19);(A5,I3,M6,E4,P20);(A5,I3,M6,E5,P1);(A5,I3,M6,E5,P2);(A5,I3,M6,E5,P3);(A5,I3,M6,E5,P4);(A5,I3,M6,E5,P5);(A5,I3,M6,E5,P6);(A5,I3,M6,E5,P7);(A5,I3,M6,E5,P8);(A5,I3,M6,E5,P9);(A5,I3,M6,E5,P10);(A5,I3,M6,E5,P11);(A5,I3,M6,E5,P12);(A5,I3,M6,E5,P13);(A5,I3,M6,E5,P14);(A5,I3,M6,E5,P15);(A5,I3,M6,E5,P16);(A5,I3,M6,E5,P17);(A5,I3,M6,E5,P18);(A5,I3,M6,E5,P19);(A5,I3,M6,E5,P20);(A5,I3,M6,E6,P1);(A5,I3,M6,E6,P2);(A5,I3,M6,E6,P3);(A5,I3,M6,E6,P4);(A5,I3,M6,E6,P5);(A5,I3,M6,E6,P6);(A5,I3,M6,E6,P7);(A5,I3,M6,E6,P8);(A5,I3,M6,E6,P9);(A5,I3,M6,E6,P10);(A5,I3,M6,E6,P11);(A5,I3,M6,E6,P12);(A5,I3,M6,E6,P13);(A5,I3,M6,E6,P14);(A5,I3,M6,E6,P15);(A5,I3,M6,E6,P16);(A5,I3,M6,E6,P17);(A5,I3,M6,E6,P18);(A5,I3,M6,E6,P19);(A5,I3,M6,E6,P20);(A5,I3,M6,E7,P1);(A5,I3,M6,E7,P2);(A5,I3,M6,E7,P3);(A5,I3,M6,E7,P4);(A5,I3,M6,E7,P5);(A5,I3,M6,E7,P6);(A5,I3,M6,E7,P7);(A5,I3,M6,E7,P8);(A5,I3,M6,E7,P9);(A5,I3,M6,E7,P10);(A5,I3,M6,E7,P11);(A5,I3,M6,E7,P12);(A5,I3,M6,E7,P13);(A5,I3,M6,E7,P14);(A5,I3,M6,E7,P15);(A5,I3,M6,E7,P16);(A5,I3,M6,E7,P17);(A5,I3,M6,E7,P18);(A5,I3,M6,E7,P19);(A5,I3,M6,E7,P20);(A5,I3,M6,E8,P1);(A5,I3,M6,E8,P2);(A5,I3,M6,E8,P3);(A5,I3,M6,E8,P4);(A5,I3,M6,E8,P5);(A5,I3,M6,E8,P6);(A5,I3,M6,E8,P7);(A5,I3,M6,E8,P8);(A5,I3 ,M6,E8,P9);(A5,I3,M6,E8,P10);(A5,I3,M6,E8,P11);(A5,I3,M6,E8,P12);(A5,I3,M6,E8,P13);(A5,I3,M6,E8,P14);(A5,I3,M6,E8,P15);(A5,I3,M6,E8,P16);(A5,I3,M6,E8,P17);(A5,I3,M6,E8,P18);(A5,I3,M6,E8,P19);(A5,I3,M6,E8,P20);(A5,I3,M6,E9,P1);(A5,I3,M6,E9,P2);(A5,I3,M6,E9,P3);(A5,I3,M6,E9,P4);(A5,I3,M6,E9,P5);(A5,I3,M6,E9,P6);(A5,I3,M6,E9,P7);(A5,I3,M6,E9,P8);(A5,I3,M6,E9,P9);(A5,I3,M6,E9,P10);(A5,I3,M6,E9,P11);(A5,I3,M6,E9,P12);(A5,I3,M6,E9,P13);(A5,I3,M6,E9,P14);(A5,I3,M6,E9,P15);(A5,I3,M6,E9,P16);(A5,I3,M6,E9,P17);(A5,I3,M6,E9,P18);(A5,I3,M6,E9,P19);(A5,I3,M6,E9,P20);(A6,I1,M1,E1,P1);(A6,I1,M1,E1,P2);(A6,I1,M1,E1,P3);(A6,I1,M1,E1,P4);(A6,I1,M1,E1,P5);(A6,I1,M1,E1,P6);(A6,I1,M1,E1,P7);(A6,I1,M1,E1,P8);(A6,I1,M1,E1,P9);(A6,I1,M1,E1,P10);(A6,I1,M1,E1,P11);(A6,I1,M1,E1,P12);(A6,I1,M1,E1,P13);(A6,I1,M1,E1,P14);(A6,I1,M1,E1,P15);(A6,I1,M1,E1,P16);(A6,I1,M1,E1,P17);(A6,I1,M1,E1,P18);(A6,I1,M1,E1,P19);(A6,I1,M1,E1,P20);(A6,I1,M1,E2,P1);(A6,I1,M1,E2,P2);(A6,I1,M1,E2,P3);(A6,I1,M1,E2,P4);(A6,I1,M1,E2,P5);(A6,I1,M1,E2,P6);(A6,I1,M1,E2,P7);(A6,I1,M1,E2,P8);(A6,I1,M1,E2,P9);(A6,I1,M1,E2,P10);(A6,I1,M1,E2,P11);(A6,I1,M1,E2,P12);(A6,I1,M1,E2,P13);(A6,I1,M1,E2,P14);(A6,I1,M1,E2,P15);(A6,I1,M1,E2,P16);(A6,I1,M1,E2,P17);(A6,I1,M1,E2,P18);(A6,I1,M1,E2,P19);(A6,I1,M1,E2,P20);(A6,I1,M1,E3,P1);(A6,I1,M1,E3,P2);(A6,I1,M1,E3,P3);(A6,I1,M1,E3,P4);(A6,I1,M1,E3,P5);(A6,I1,M1,E3,P6);(A6,I1,M1,E3,P7);(A6,I1,M1,E3,P8);(A6,I1,M1,E3,P9);(A6,I1,M1,E3,P10);(A6,I1,M1,E3,P11);(A6,I1,M1,E3,P12);(A6,I1,M1,E3,P13);(A6,I1,M1,E3,P14);(A6,I1,M1,E3,P15);(A6,I1,M1,E3,P16);(A6,I1,M1,E3,P17);(A6,I1,M1,E3,P18);(A6,I1,M1,E3,P19);(A6,I1,M1,E3,P20);(A6,I1,M1,E4,P1);(A6,I1,M1,E4,P2);(A6,I1,M1,E4,P3);(A6,I1,M1,E4,P4);(A6,I1,M1,E4,P5);(A6,I1,M1,E4,P6);(A6,I1,M1,E4,P7);(A6,I1,M1,E4,P8);(A6,I1,M1,E4,P9);(A6,I1,M1,E4,P10);(A6,I1,M1,E4,P11);(A6,I1,M1,E4,P12);(A6,I1,M1,E4,P13);(A6,I1,M1,E4,P14);(A6,I1,M1,E4,P15);(A6,I1,M1,E4,P16);(A6,I1,M1,E4,P17);(A6,I1,M1,E4,P18);(A6,I1,M1,E4,P19);(A6,I1,M1,E4,P20);(A6,I1,M1,E5,P1);(A6,I1,M1,E5,P2);(A6,I1,M1,E5,P3);(A6,I1,M1,E5,P4);(A6,I1,M1,E5,P5);(A6,I1,M1,E5,P6);(A6,I1,M1,E5,P7);(A6,I1,M1,E5,P8);(A6,I1,M1,E5,P9);(A6,I1,M1,E5,P10);(A6,I1,M1,E5,P11);(A6,I1,M1,E5,P12);(A6,I1,M1,E5,P13);(A6,I1,M1,E5,P14);(A6,I1,M1,E5,P15);(A6,I1,M1,E5,P16);(A6,I1,M1,E5,P17);(A6,I1,M1,E5,P18);(A6,I1,M1,E5,P19);(A6,I1,M1,E5,P20);(A6,I1,M1,E6,P1);(A6,I1,M1,E6,P2);(A6,I1,M1,E6,P3);(A6,I1,M1,E6,P4);(A6,I1,M1,E6,P5);(A6,I1,M1,E6,P6);(A6,I1,M1,E6,P7);(A6,I1,M1,E6,P8);(A6,I1,M1,E6,P9);(A6,I1,M1,E6,P10);(A6,I1,M1,E6,P11);(A6,I1,M1,E6,P12);(A6,I1,M1,E6,P13);(A6,I1,M1,E6,P14);(A6,I1,M1,E6,P15);(A6,I1,M1,E6,P16);(A6,I1,M1,E6,P17);(A6,I1,M1,E6,P18);(A6,I1,M1,E6,P19);(A6,I1,M1,E6,P20);(A6,I1,M1,E7,P1);(A6,I1,M1,E7,P2);(A6,I1,M1,E7,P3);(A6,I1,M1,E7,P4);(A6,I1,M1,E7,P5);(A6,I1,M1,E7,P6);(A6,I1,M1,E7,P7);(A6,I1,M1,E7,P8);(A6,I1,M1,E7,P9);(A6,I1,M1,E7,P10);(A6,I1,M1,E7,P11);(A6,I1,M1,E7,P12);(A6,I1,M1,E7,P13);(A6,I1,M1,E7,P14);(A6,I1,M1,E7,P15);(A6,I1,M1,E7,P16);(A6,I1,M1,E7,P17);(A6,I1,M1,E7,P18);(A6,I1,M1,E7,P19);(A6,I1,M1,E7,P20);(A6,I1,M1,E8,P1);(A6,I1,M1,E8,P2);(A6,I1,M1,E8,P3);(A6,I1,M1,E8,P4);(A6,I1,M1,E8,P5);(A6,I1,M1,E8,P6);(A6,I1,M1,E8,P7);(A6,I1,M1,E8,P8);(A6,I1,M1,E8,P9);(A6,I1,M1,E8,P10);(A6,I1,M1,E8,P11);(A6,I1,M1,E8,P12);(A6,I1,M1,E8,P13);(A6,I1,M1,E8,P14);(A6,I1,M1,E8,P15);(A6,I1,M1,E8,P16);(A6,I1,M1,E8,P17);(A6,I1,M1,E8,P18);(A6,I1,M1,E8,P19);(A6,I1,M1,E8,P20);(A6,I1,M1,E9,P1);(A6,I1,M1,E9,P2);(A6,I1,M1,E9,P3);(A6,I1,M1,E9,P4);(A6,I1,M1,E9,P5);(A6,I1,M1,E9,P6);(A6,I1,M1,E9,P7);(A6,I1,M1,E9,P8);(A6,I1,M1,E9,P9);(A6,I1,M1,E9,P10);(A6,I1,M1,E9,P11);(A6,I1,M1,E9,P12);(A6,I1,M1,E9,P13);(A6,I1,M1,E9,P14);(A6,I1,M1,E9,P15);(A6,I1,M1,E9,P16);(A6,I1,M1,E9,P17);(A6,I1,M1,E9,P18);(A6,I1,M1,E9,P19);(A6,I1,M1,E9,P20);(A6,I1,M2,E1,P1);(A6,I1,M2,E1,P2);(A6,I1,M2,E1,P3);(A6,I1,M2,E1,P4);(A6,I1,M2,E1,P5);(A6,I1,M2,E1,P6);(A6,I1,M2,E1,P7);(A6,I1,M2,E1,P8);(A6,I1,M2,E1,P9);(A6,I1,M2,E1,P10);(A6,I1,M2,E1,P11);(A6,I1,M2,E1,P12);(A6,I1,M2,E1,P13);(A6,I1,M2,E1,P14);(A6,I1,M2,E1,P15);(A6,I1,M2,E1,P16);(A6,I1,M2,E1,P17);(A6,I1,M2,E1,P18);(A6,I1,M2,E1,P19);(A6,I1,M2,E1,P20);(A6,I1,M2,E2,P1);(A6,I1,M2,E2,P2);(A6,I1,M2,E2,P3);(A6,I1,M2,E2,P4);(A6,I1,M2,E2,P5);(A6,I1,M2,E2,P6);(A6,I1,M2,E2,P7);(A6,I1,M2,E2,P8);(A6,I1,M2,E2,P9);(A6,I1,M2,E2,P10);(A6,I1,M2,E2,P11);(A6,I1,M2,E2,P12);(A6,I1,M2,E2,P13);(A6,I1,M2,E2,P14);(A6,I1,M2,E2,P15);(A6,I1,M2,E2,P16);(A6,I1,M2,E2,P17);(A6,I1,M2,E2,P18);(A6,I1,M2,E2,P19);(A6,I1,M2,E2,P20);(A6,I1,M2,E3,P1);(A6,I1,M2,E3,P2);(A6,I1,M2,E3,P3);(A6,I1,M2,E3,P4);(A6,I1,M2,E3,P5);(A6,I1,M2,E3,P6);(A6,I1,M2,E3,P7);(A6,I1,M2,E3,P8);(A6,I1,M2,E3,P9);(A6,I1,M2,E3,P10);(A6,I1,M2,E3,P11);(A6,I1,M2,E3,P12);(A6,I1,M2,E3,P13);(A6,I1,M2,E3,P14);(A6,I1,M2,E3,P15);(A6,I1,M2,E3,P16);(A6,I1,M2,E3,P17);(A6,I1,M2,E3,P18);(A6,I1,M2,E3,P19);(A6,I1,M2,E3,P20);(A6,I1,M2,E4,P1);(A6,I1,M2,E4,P2);(A6,I1,M2,E4,P3);(A6,I1,M2,E4,P4);(A6,I1,M2,E4,P5);(A6,I1,M2,E4,P6);(A6,I1,M2,E4,P7);(A6,I1,M2,E4,P8);(A6,I1,M2,E4,P9);(A6,I1,M2,E4,P10);(A6,I1,M2,E4,P11);(A6,I1,M2,E4,P12);(A6,I1,M2,E4,P13);(A6,I1,M2,E4,P14);(A6,I1,M2,E4,P15);(A6,I1,M2,E4,P16);(A6,I1,M2,E4,P17);(A6,I1,M2,E4,P18);(A6,I1,M2,E4,P19);(A6,I1,M2,E4,P20);(A6,I1,M2,E5,P1);(A6,I1,M2,E5,P2);(A6,I1,M2,E5,P3);(A6,I1,M2,E5,P4);(A6,I1,M2,E5,P5);(A6,I1,M2,E5,P6);(A6,I1,M2,E5,P7);(A6,I1,M2,E5,P8);(A6,I1,M2,E5,P9);(A6,I1,M2,E5,P10);(A6,I1,M2,E5,P11);(A6,I1,M2,E5,P12);(A6,I1,M2,E5,P13);(A6,I1,M2,E5,P14);(A6,I1,M2,E5,P15);(A6,I1,M2,E5,P16);(A6,I1,M2,E5,P17);(A6,I1,M2,E5,P18);(A6,I1,M2,E5,P19);(A6,I1,M2,E5,P20);(A6,I1,M2,E6,P1);(A6,I1,M2,E6,P2);(A6,I1,M2,E6,P3);(A6,I1,M2,E6,P4);(A6,I1,M2,E6,P5);(A6,I1,M2,E6,P6);(A6,I1,M2,E6,P7);(A6,I1,M2,E6,P8);(A6,I1,M2,E6,P9);(A6,I1,M2,E6,P10);(A6,I1,M2,E6,P11);(A6,I1,M2,E6,P12);(A6,I1,M2,E6,P13);(A6,I1,M2,E6,P14);(A6,I1,M2,E6,P15);(A6,I1,M2,E6,P16);(A6,I1,M2,E6,P17);(A6,I1,M2,E6,P18);(A6,I1,M2,E6,P19);(A6,I1,M2,E6,P20);(A6,I1,M2,E7,P1);(A6,I1,M2,E7,P2);(A6,I1,M2,E7,P3);(A6,I1,M2,E7,P4);(A6,I1,M2,E7,P5);(A6,I1,M2,E7,P6);(A6,I1,M2,E7,P7);(A6,I1,M2,E7,P8);(A6,I1,M2,E7,P9);(A6,I1,M2,E7,P10);(A6,I1,M2,E7,P11);(A6,I1,M2,E7,P12);(A6,I1,M2,E7,P13);(A6,I1,M2,E7,P14);(A6,I1,M2,E7,P15);(A6,I1,M2,E7,P16);(A6,I1,M2,E7,P17);(A6,I1,M2,E7,P18);(A6,I1,M2,E7,P19);(A6,I1,M2,E7,P20);(A6,I1,M2,E8,P1);(A6,I1,M2,E8,P2);(A6,I1,M2,E8,P3);(A6,I1,M2,E8,P4);(A6,I1,M2,E8,P5);(A6,I1,M2,E8,P6);(A6,I1,M2,E8,P7);(A6,I1,M2,E8,P8);(A6,I1,M2,E8,P9);(A6,I1,M2,E8,P10);(A6,I1,M2,E8,P11);(A6,I1,M2,E8,P12);(A6,I1,M2,E8,P13);(A6,I1,M2,E8,P14);(A6,I1,M2,E8,P15);(A6,I1,M2,E8,P16);(A6,I1,M2,E8,P17);(A6,I1,M2,E8,P18);(A6,I1,M2,E8,P19);(A6,I1,M2,E8,P20);(A6,I1,M2,E9,P1);(A6,I1,M2,E9,P2);(A6,I1,M2,E9,P3);(A6,I1,M2,E9,P4);(A6,I1,M2,E9,P5);(A6,I1,M2,E9,P6);(A6,I1,M2,E9,P7);(A6,I1,M2,E9,P8);(A6,I1,M2,E9,P9);(A6,I1,M2,E9,P10);(A6,I1,M2,E9,P11);(A6,I1,M2,E9,P12);(A6,I1,M2,E9,P13);(A6,I1,M2,E9,P14);(A6,I1,M2,E9,P15);(A6,I1,M2,E9,P16);(A6,I1,M2,E9,P17);(A6,I1,M2,E9,P18);(A6,I1,M2,E9,P19);(A6,I1,M2,E9,P20);(A6,I1,M3,E1,P1);(A6,I1,M3,E1,P2);(A6,I1,M3,E1,P3);(A6,I1,M3,E1,P4);(A6,I1,M3,E1,P5);(A6,I1,M3,E1,P6);(A6,I1,M3,E1,P7);(A6,I1,M3,E1,P8);(A6,I1,M3,E1,P9);(A6,I1,M3,E1,P10);(A6,I1,M3,E1,P11);(A6,I1,M3,E1,P12);(A6,I1,M3,E1,P13);(A6,I1,M3,E1,P14);(A6,I1,M3,E1,P15);(A6,I1,M3,E1,P16);(A6,I1,M3,E1,P17);(A6,I1,M3,E1,P18);(A6,I1,M3,E1,P19);(A6,I1,M3,E1,P20);(A6,I1,M3,E2,P1);(A6,I1,M3,E2,P2);(A6,I1,M3,E2,P3);(A6,I1,M3,E2,P4);(A6,I1,M3,E2,P5);(A6,I1,M3,E2,P6);(A6,I1,M3,E2,P7);(A6,I1,M3,E2,P8);(A6,I1,M3,E2,P9);(A6,I1,M3,E2,P10);(A6,I1,M3,E2,P11);(A6,I1,M3,E2,P12);(A6,I1,M3,E2,P13);(A6,I1,M3,E2,P14);(A6,I1,M3,E2,P15);(A6,I1,M3,E2,P16);(A6,I1,M3,E2,P17);(A6,I1,M3,E2,P18);(A6,I1,M3,E2,P19);(A6,I1,M3,E2,P20);(A6,I1,M3,E3,P1);(A6,I1,M3,E3,P2);(A6,I1,M3,E3,P3);(A6,I1,M3,E3,P4);(A6,I1,M3,E3,P5);(A6,I1,M3,E3,P6);(A6,I1,M3,E3,P7);(A6,I1,M3,E3,P8);(A6,I1,M3,E3,P9);(A6,I1,M3,E3,P10);(A6,I1,M3,E3,P11);(A6,I1,M3,E3,P12);(A6,I1,M3,E3,P13);(A6,I1,M3,E3,P14);(A6,I1,M3,E3,P15);(A6,I1,M3,E3,P16);(A6,I1,M3,E3,P17);(A6,I1,M3,E3,P18);(A6,I1,M3,E3,P19);(A6,I1,M3,E3,P20);(A6,I1,M3,E4,P1);(A6,I1,M3,E4,P2);(A6,I1,M3,E4,P3);(A6,I1,M3,E4,P4);(A6,I1,M3,E4,P5);(A6,I1,M3,E4,P6);(A6,I1,M3,E4,P7);(A6,I1,M3,E4,P8);(A6,I1,M3,E4,P9);(A6,I1,M3,E4,P10);(A6,I1,M3,E4,P11);(A6,I1,M3,E4,P12);(A6,I1,M3,E4,P13);(A6,I1,M3,E4,P14);(A6,I1,M3,E4,P15);(A6,I1,M3,E4,P16);(A6,I1,M3,E4,P17);(A6,I1,M3,E4,P18);(A6,I1,M3,E4,P19);(A6,I1,M3,E4,P20);(A6,I1,M3,E5,P1);(A6,I1,M3,E5,P2);(A6,I1,M3,E5,P3);(A6,I1,M3,E5,P4);(A6,I1,M3,E5,P5);(A6,I1,M3,E5,P6);(A6,I1,M3,E5,P7);(A6,I1,M3,E5,P8);(A6,I1,M3,E5,P9);(A6,I1,M3,E5,P10);(A6,I1,M3,E5,P11);(A6,I1,M3,E5,P12);(A6,I1,M3,E5,P13);(A6,I1,M3,E5,P14);(A6,I1,M3,E5,P15);(A6,I1,M3,E5,P16);(A6,I1,M3,E5,P17);(A6,I1,M3,E5,P18);(A6,I1,M3,E5,P19);(A6,I1,M3,E5,P20);(A6,I1,M3,E6,P1);(A6,I1,M3,E6,P2);(A6,I1,M3,E6,P3);(A6,I1,M3,E6,P4);(A6,I1,M3,E6,P5);(A6,I1,M3,E6,P6);(A6,I1,M3,E6,P7);(A6,I1,M3,E6,P8);(A6,I1,M3,E6,P9);(A6,I1,M3,E6,P10);(A6,I1,M3,E6,P11);(A6,I1,M3,E6,P12);(A6,I1,M3,E6,P13);(A6,I1,M3,E6,P14);(A6,I1,M3,E6,P15);(A6,I1,M3,E6,P16);(A6,I1,M3,E6,P17);(A6,I1,M3,E6,P18);(A6,I1,M3,E6,P19);(A6,I1,M3,E6,P20);(A6,I1,M3,E7,P1);(A6,I1,M3,E7,P2);(A6,I1,M3,E7,P3);(A6,I1,M3,E7,P4);(A6,I1,M3,E7,P5);(A6,I1,M3,E7,P6);(A6,I1,M3,E7,P7);(A6,I1,M3,E7,P8);(A6,I1,M3,E7,P9);(A6,I1,M3,E7,P10);(A6,I1,M3,E7,P11);(A6,I1,M3,E7,P12);(A6,I1,M3,E7,P13);(A6,I1,M3,E7,P14);(A6,I1,M3,E7,P15);(A6,I1,M3,E7,P16);(A6,I1,M3,E7,P17);(A6,I1,M3,E7,P18);(A6,I1,M3,E7,P19);(A6,I1,M3,E7,P20);(A6,I1,M3,E8,P1);(A6,I1,M3,E8,P2);(A6,I1,M3,E8,P3);(A6,I1,M3,E8,P4);(A6,I1,M3,E8,P5);(A6,I1,M3,E8,P6);(A6,I1,M3,E8,P7);(A6,I1,M3,E8,P8);(A6,I1,M3,E8,P9);(A6,I1,M3,E8,P10);(A6,I1,M3,E8,P11);(A6,I1,M3,E8,P12);(A6,I1,M3,E8,P13);(A6,I1,M3,E8,P14);(A6,I1,M3,E8,P15);(A6,I1,M3,E8,P16);(A6,I1,M3,E8,P17);(A6,I1,M3,E8,P18);(A6,I1,M3,E8,P19);(A6,I1,M3,E8,P20);(A6,I1,M3,E9,P1);(A6,I1,M3,E9,P2);(A6,I1,M3,E9,P3);(A6,I1,M3,E9,P4);(A6,I1,M3,E9,P5);(A6,I1,M3,E9,P6);(A6,I1,M3,E9,P7);(A6,I1,M3,E9,P8);(A6,I1,M3,E9,P9);(A6,I1,M3,E9,P10);(A6,I1,M3,E9,P11);(A6,I1,M3,E9,P12);(A6,I1,M3,E9,P13);(A6,I1,M3,E9,P14);(A6,I1,M3,E9,P15);(A6,I1,M3,E9,P16);(A6,I1,M3,E9,P17);(A6,I1,M3,E9,P18);(A6,I1,M3,E9,P19);(A6,I1,M3,E9,P20);(A6,I1,M4,E1,P1);(A6,I1,M4,E1,P2);(A6,I1,M4,E1,P3);(A6,I1,M4,E1,P4);(A6,I1,M4,E1,P5);(A6,I1,M4,E1,P6);(A6,I1,M4,E1,P7);(A6,I1,M4,E1,P8);(A6,I1,M4,E1,P9);(A6,I1,M4,E1,P10);(A6,I1,M4,E1,P11);(A6,I1,M4,E1,P12);(A6,I1,M4,E1,P13);(A6,I1,M4,E1,P14);(A6,I1,M4,E1,P15);(A6,I1,M4,E1,P16);(A6,I1,M4,E1,P17);(A6,I1,M4,E1,P18);(A6,I1,M4,E1,P19);(A6,I1,M4,E1,P20);(A6,I1,M4,E2,P1);(A6,I1,M4,E2,P2);(A6,I1,M4,E2,P3);(A6,I1,M4,E2,P4);(A6,I1,M4,E2,P5);(A6,I1,M4,E2,P6);(A6,I1,M4,E2,P7);(A6,I1,M4,E2,P8);(A6,I1,M4,E2,P9);(A6,I1,M4,E2,P10);(A6,I1,M4,E2,P11);(A6,I1,M4,E2,P12);(A6,I1,M4,E2,P13);(A6,I1,M4,E2,P14);(A6,I1,M4,E2,P15);(A6,I1,M4,E2,P16);(A6,I1,M4,E2,P17);(A6,I1,M4,E2,P18);(A6,I1,M4,E2,P19);(A6,I1,M4,E2,P20);(A6,I1,M4,E3,P1);(A6,I1,M4,E3,P2);(A6,I1,M4,E3,P3);(A6,I1,M4,E3,P4);(A6,I1,M4,E3,P5);(A6,I1,M4,E3,P6);(A6,I1,M4,E3,P7);(A6,I1,M4,E3,P8);(A6,I1,M4,E3,P9);(A6,I1,M4,E3,P10);(A6,I1,M4,E3,P11);(A6,I1,M4,E3,P12);(A6,I1,M4,E3,P13);(A6,I1,M4,E3,P14);(A6,I1,M4,E3,P15);(A6,I1,M4,E3,P16);(A6,I1,M4,E3,P17);(A6,I1,M4,E3,P18);(A6,I1,M4,E3,P19);(A6,I1,M4,E3,P20);(A6,I1,M4,E4,P1);(A6,I1,M4,E4,P2);(A6,I1,M4,E4,P3);(A6,I1,M4,E4,P4);(A6,I1,M4,E4,P5);(A6,I1,M4,E4,P6);(A6,I1,M4,E4,P7);(A6,I1,M4,E4,P8);(A6,I1,M4,E4,P9);(A6,I1,M4,E4,P10);(A6,I1,M4,E4,P11);(A6,I1,M4,E4,P12);(A6,I1,M4,E4,P13);(A6,I1,M4,E4,P14);(A6,I1,M4,E4,P15);(A6,I1,M4,E4,P16);(A6,I1,M4,E4,P17);(A6,I1,M4,E4,P18);(A6,I1,M4,E4,P19);(A6,I1,M4,E4,P20);(A6,I1,M4,E5,P1);(A6,I1,M4,E5,P2);(A6,I1,M4,E5,P3);(A6,I1,M4,E5,P4);(A6,I1,M4,E5,P5);(A6,I1,M4,E5,P6);(A6,I1,M4,E5,P7);(A6,I1,M4,E5,P8);(A6,I1,M4,E5,P9);(A6,I1,M4,E5,P10);(A6,I1,M4,E5,P11);(A6,I1,M4,E5,P12);(A6,I1,M4,E5,P13);(A6,I1,M4,E5,P14);(A6,I1,M4,E5,P15);(A6,I1,M4,E5,P16);(A6,I1,M4,E5,P17);(A6,I1,M4,E5,P18);(A6,I1,M4,E5,P19);(A6,I1,M4,E5,P20);(A6,I1,M4,E6,P1);(A6,I1,M4,E6,P2);(A6,I1,M4,E6,P3);(A6,I1,M4,E6,P4);(A6,I1,M4,E6,P5);(A6,I1,M4,E6,P6);(A6,I1,M4,E6,P7);(A6,I1,M4,E6,P8);(A6,I1,M4,E6,P9);(A6,I1,M4,E6,P10);(A6,I1,M4,E6,P11);(A6,I1,M4,E6,P12);(A6,I1,M4,E6,P13);(A6,I1,M4,E6,P14);(A6,I1,M4,E6,P15);(A6,I1,M4,E6,P16);(A6,I1,M4,E6,P17);(A6,I1,M4,E6,P18);(A6,I1,M4,E6,P19);(A6,I1,M4,E6,P20);(A6,I1,M4,E7,P1);(A6,I1,M4,E7,P2);(A6,I1,M4,E7,P3);(A6,I1,M4,E7,P4);(A6,I1,M4,E7,P5);(A6,I1,M4,E7,P6);(A6,I1,M4,E7,P7);(A6,I1,M4,E7,P8);(A6,I1,M4,E7,P9);(A6,I1,M4,E7,P10);(A6,I1,M4,E7,P11);(A6,I1,M4,E7,P12);(A6,I1,M4,E7,P13);(A6,I1,M4,E7,P14);(A6,I1,M4,E7,P15);(A6,I1,M4,E7,P16);(A6,I1,M4,E7,P17);(A6,I1,M4,E7,P18);(A6,I1,M4,E7,P19);(A6,I1,M4,E7,P20);(A6,I1,M4,E8,P1);(A6,I1,M4,E8,P2);(A6,I1,M4,E8,P3);(A6,I1,M4,E8,P4);(A6,I1,M4,E8,P5);(A6,I1,M4,E8,P6);(A6,I1,M4,E8,P7);(A6,I1,M4,E8,P8);(A6,I1,M4,E8,P9);(A6,I1,M4,E8,P10);(A6,I1,M4,E8,P11);(A6,I1,M4,E8,P12);(A6,I1,M4,E8,P13);(A6,I1,M4,E8,P14);(A6,I1,M4,E8,P15);(A6,I1,M4,E8,P16);(A6,I1,M4,E8,P17);(A6,I1,M4,E8,P18);(A6,I1,M4,E8,P19);(A6,I1,M4,E8,P20);(A6,I1,M4,E9,P1);(A6,I1,M4,E9,P2);(A6,I1,M4,E9,P3);(A6,I1,M4,E9,P4);(A6,I1,M4,E9,P5);(A6,I1,M4,E9,P6);(A6,I1,M4,E9,P7);(A6,I1,M4,E9,P8);(A6,I1,M4,E9,P9);(A6,I1,M4,E9,P10);(A6,I1,M4,E9,P11);(A6,I1,M4,E9,P12);(A6,I1,M4,E9,P13);(A6,I1,M4,E9,P14);(A6,I1,M4,E9,P15);(A6,I1,M4,E9,P16);(A6,I1,M4,E9,P17);(A6,I1,M4,E9,P18);(A6,I1,M4,E9,P19);(A6,I1,M4,E9,P20);(A6,I1,M5,E1,P1);(A6,I1,M5,E1,P2);(A6,I1,M5,E1,P3);(A6,I1,M5,E1,P4);(A6,I1,M5,E1,P5);(A6,I1,M5,E1,P6);(A6,I1,M5,E1,P7);(A6,I1,M5,E1,P8);(A6,I1,M5,E1,P9);(A6,I1,M5,E1,P10);(A6,I1,M5,E1,P11);(A6,I1,M5,E1,P12);(A6,I1,M5,E1,P13);(A6,I1,M5,E1,P14);(A6,I1,M5,E1,P15);(A6,I1,M5,E1,P16);(A6,I1,M5,E1,P17);(A6,I1,M5,E1,P18);(A6,I1,M5,E1,P19);(A6,I1,M5,E1,P20);(A6,I1,M5,E2,P1);(A6,I1,M5,E2,P2);(A6,I1,M5,E2,P3);(A6,I1,M5,E2,P4);(A6,I1,M5,E2,P5);(A6,I1,M5,E2,P6);(A6,I1,M5,E2,P7);(A6,I1,M5,E2,P8);(A6,I1,M5,E2,P9);(A6,I1,M5,E2,P10);(A6,I1,M5,E2,P11);(A6,I1,M5,E2,P12);(A6,I1,M5,E2,P13);(A6,I1,M5,E2,P14);(A6,I1,M5,E2,P15);(A6,I1,M5,E2,P16);(A6,I1,M5,E2,P17);(A6,I1,M5,E2,P18);(A6,I1,M5,E2,P19);(A6,I1,M5,E2,P20);(A6,I1,M5,E3,P1);(A6,I1,M5,E3,P2);(A6,I1,M5,E3,P3);(A6,I1,M5,E3,P4);(A6,I1,M5,E3,P5);(A6,I1,M5,E3,P6);(A6,I1,M5,E3,P7);(A6,I1,M5,E3,P8);(A6,I1,M5,E3,P9);(A6,I1,M5,E3,P10);(A6,I1,M5,E3,P11);(A6,I1,M5,E3,P12);(A6,I1,M5,E3,P13);(A6,I1,M5,E3,P14);(A6,I1,M5,E3,P15);(A6,I1,M5,E3,P16);(A6,I1,M5,E3,P17);(A6,I1,M5,E3,P18);(A6,I1,M5,E3,P19);(A6,I1,M5,E3,P20);(A6,I1,M5,E4,P1);(A6,I1,M5,E4,P2);(A6,I1,M5,E4,P3);(A6,I1,M5,E4,P4);(A6,I1,M5,E4,P5);(A6,I1,M5,E4,P6);(A6,I1,M5,E4,P7);(A6,I1,M5,E4,P8);(A6,I1,M5,E4,P9);(A6,I1,M5,E4,P10);(A6,I1,M5,E4,P11);(A6,I1,M5,E4,P12);(A6,I1,M5,E4,P13);(A6,I1,M5,E4,P14);(A6,I1,M5,E4,P15);(A6,I1,M5,E4,P16);(A6,I1,M5,E4,P17);(A6,I1,M5,E4,P18);(A6,I1,M5,E4,P19);(A6,I1,M5,E4,P20);(A6,I1,M5,E5,P1);(A6,I1,M5,E5,P2);(A6,I1,M5,E5,P3);(A6,I1,M5,E5,P4);(A6,I1,M5,E5,P5);(A6,I1,M5,E5,P6);(A6,I1,M5,E5,P7);(A6,I1,M5,E5,P8);(A6,I1,M5,E5,P9);(A6,I1,M5,E5,P10);(A6,I1,M5,E5,P11);(A6,I1,M5,E5,P12);(A6,I1,M5,E5,P13);(A6,I1,M5,E5,P14);(A6,I1,M5,E5,P15);(A6,I1,M5,E5,P16);(A6,I1,M5,E5,P17);(A6,I1,M5,E5,P18);(A6,I1,M5,E5,P19);(A6,I1,M5,E5,P20);(A6,I1,M5,E6,P1);(A6,I1,M5,E6,P2);(A6,I1,M5,E6,P3);(A6,I1,M5,E6,P4);(A6,I1,M5,E6,P5);(A6,I1,M5,E6,P6);(A6,I1,M5,E6,P7);(A6,I1,M5,E6,P8);(A6,I1,M5,E6,P9);(A6,I1,M5,E6,P10);(A6,I1,M5,E6,P11);(A6,I1,M5,E6,P12);(A6,I1,M5,E6,P13);(A6,I1,M5,E6,P14);(A6,I1,M5,E6,P15);(A6,I1,M5,E6,P16);(A6,I1,M5,E6,P17);(A6,I1,M5,E6,P18);(A6,I1,M5,E6,P19);(A6,I1,M5,E6,P20);(A6,I1,M5,E7,P1);(A6,I1,M5,E7,P2);(A6,I1,M5,E7,P3);(A6,I1,M5,E7,P4);(A6,I1,M5,E7,P5);(A6,I1,M5,E7,P6);(A6,I1,M5,E7,P7);(A6,I1,M5,E7,P8);(A6,I1,M5,E7,P9);(A6,I1,M5,E7,P10);(A6,I1,M5,E7,P11);(A6,I1,M5,E7,P12);(A6,I1,M5,E7,P13);(A6,I1,M5,E7,P14);(A6,I1,M5,E7,P15);(A6,I1,M5,E7,P16);(A6,I1,M5,E7,P17);(A6,I1,M5,E7,P18);(A6,I1,M5,E7,P19);(A6,I1,M5,E7,P20);(A6,I1,M5,E8,P1);(A6,I1,M5,E8,P2);(A6,I1,M5,E8,P3);(A6,I1,M5,E8,P4);(A6,I1,M5,E8,P5);(A6,I1,M5,E8,P6);(A6,I1,M5,E8,P7);(A6,I1,M5,E8,P8);(A6,I1,M5,E8,P9);(A6,I1,M5,E8,P10);(A6,I1,M5,E8,P11);(A6,I1,M5,E8,P12);(A6,I1,M5,E8,P13);(A6,I1,M5,E8,P14);(A6,I1,M5,E8,P15);(A6,I1,M5,E8,P16);(A6,I1,M5,E8,P17);(A6,I1,M5,E8,P18);(A6,I1,M5,E8,P19);(A6,I1,M5,E8,P20);(A6,I1,M5,E9,P1);(A6,I1,M5,E9,P2);(A6,I1,M5,E9,P3);(A6,I1,M5,E9,P4);(A6,I1,M5,E9,P5);(A6,I1,M5,E9,P6);(A6,I1,M5,E9,P7);(A6,I1,M5,E9,P8);(A6,I1,M5,E9,P9);(A6,I1,M5,E9,P10);(A6,I1,M5,E9,P11);(A6,I1,M5,E9,P12);(A6,I1,M5,E9,P13);(A6,I1,M5,E9,P14);(A6,I1,M5,E9,P15);(A6,I1,M5,E9,P16);(A6,I1,M5,E9,P17);(A6,I1,M5,E9,P18);(A6,I1,M5,E9,P19);(A6,I1,M5,E9,P20);(A6,I1,M6,E1,P1);(A6,I1,M6,E1,P2);(A6,I1,M6,E1,P3);(A6,I1,M6,E1,P4);(A6,I1,M6,E1,P5);(A6,I1,M6,E1,P6);(A6,I1,M6,E1,P7);(A6,I1,M6,E1,P8);(A6,I1,M6,E1,P9);(A6,I1,M6,E1,P10);(A6,I1,M6,E1,P11);(A6,I1,M6,E1,P12);(A6,I1,M6,E1,P13);(A6,I1,M6,E1,P14);(A6,I1,M6,E1,P15);(A6,I1,M6,E1,P16);(A6,I1,M6,E1,P17);(A6,I1,M6,E1,P18);(A6,I1,M6,E1,P19);(A6,I1,M6,E1,P20);(A6,I1,M6,E2,P1);(A6,I1,M6,E2,P2);(A6,I1,M6,E2,P3);(A6,I1,M6,E2,P4);(A6,I1,M6,E2,P5);(A6,I1,M6,E2,P6);(A6,I1,M6,E2,P7);(A6,I1,M6,E2,P8);(A6,I1,M6,E2,P9);(A6,I1,M6,E2,P10);(A6,I1,M6,E2,P11);(A6,I1,M6,E2,P12);(A6,I1,M6,E2,P13);(A6,I1,M6,E2,P14);(A6,I1,M6,E2,P15);(A6,I1,M6,E2,P16);(A6,I1,M6,E2,P17);(A6,I1,M6,E2,P18);(A6,I1,M6,E2,P19);(A6,I1,M6,E2,P20);(A6,I1,M6,E3,P1);(A6,I1,M6,E3,P2);(A6,I1,M6,E3,P3);(A6,I1,M6,E3,P4);(A6,I1,M6,E3,P5);(A6,I1,M6,E3,P6);(A6,I1,M6,E3,P7);(A6,I1,M6,E3,P8);(A6,I1,M6,E3,P9);(A6,I1,M6,E3,P10);(A6,I1,M6,E3,P11);(A6,I1,M6,E3,P12);(A6,I1,M6,E3,P13);(A6,I1,M6,E3,P14);(A6,I1,M6,E3,P15);(A6,I1,M6,E3,P16);(A6,I1,M6,E3,P17);(A6,I1,M6,E3,P18);(A6,I1,M6,E3,P19);(A6,I1,M6,E3,P20);(A6,I1,M6,E4,P1);(A6,I1,M6,E4,P2);(A6,I1,M6,E4,P3);(A6,I1,M6,E4,P4);(A6,I1,M6,E4,P5);(A6,I1,M6,E4,P6);(A6,I1,M6,E4,P7);(A6,I1,M6,E4,P8);(A6,I1,M6,E4,P9);(A6,I1,M6,E4,P10);(A6,I1,M6,E4,P11);(A6,I1,M6,E4,P12);(A6,I1,M6,E4,P13);(A6,I1,M6,E4,P14);(A6,I1,M6,E4,P15);(A6,I1,M6,E4,P16);(A6,I1,M6,E4,P17);(A6,I1,M6,E4,P18);(A6,I1,M6,E4,P19);(A6,I1,M6,E4,P20);(A6,I1,M6,E5,P1);(A6,I1,M6,E5,P2);(A6,I1,M6,E5,P3);(A6,I1,M6,E5,P4);(A6,I1,M6,E5,P5);(A6,I1,M6,E5,P6);(A6,I1,M6,E5,P7);(A6,I1,M6,E5,P8);(A6,I1,M6,E5,P9);(A6,I1,M6,E5,P10);(A6,I1,M6,E5,P11);(A6,I1,M6,E5,P12);(A6,I1,M6,E5,P13);(A6,I1,M6,E5,P14);(A6,I1,M6,E5,P15);(A6,I1,M6,E5,P16);(A6,I1,M6,E5,P17);(A6,I1,M6,E5,P18);(A6,I1,M6,E5,P19);(A6,I1,M6,E5,P20);(A6,I1,M6,E6,P1);(A6,I1,M6,E6,P2);(A6,I1,M6,E6,P3);(A6,I1,M6,E6,P4);(A6,I1,M6,E6,P5);(A6,I1,M6,E6,P6);(A6,I1,M6,E6,P7);(A6,I1,M6,E6,P8);(A6,I1,M6,E6,P9);(A6,I1,M6,E6,P10);(A6,I1,M6,E6,P11);(A6,I1,M6,E6,P12);(A6,I1,M6,E6,P13);(A6,I1,M6,E6,P14);(A6,I1,M6,E6,P15);(A6,I1,M6,E6,P16);(A6,I1,M6,E6,P17);(A6,I1,M6,E6,P18);(A6,I1,M6,E6,P19);(A6,I1,M6,E6,P20);(A6,I1,M6,E7,P1);(A6,I1,M6,E7,P2);(A6,I1,M6,E7,P3);(A6,I1,M6,E7,P4);(A6,I1,M6,E7,P5);(A6,I1,M6,E7,P6);(A6,I1,M6,E7,P7);(A6,I1,M6,E7,P8);(A6,I1,M6,E7,P9);(A6,I1,M6,E7,P10);(A6,I1,M6,E7,P11);(A6,I1,M6,E7,P12);(A6,I1,M6,E7,P13);(A6,I1,M6,E7,P14);(A6,I1,M6,E7,P15);(A6,I1,M6,E7,P16);(A6,I1,M6,E7,P17);(A6,I1,M6,E7,P18);(A6,I1,M6,E7,P19);(A6,I1,M6,E7,P20);(A6,I1,M6,E8,P1);(A6,I1,M6,E8,P2);(A6,I1,M6,E8,P3);(A6,I1,M6,E8,P4);(A6,I1,M6,E8,P5);(A6,I1,M6,E8,P6);(A6,I1,M6,E8,P7);(A6,I1,M6,E8,P8);(A6,I1,M6,E8,P9);(A6,I1,M6,E8,P10);(A6,I1,M6,E8,P11);(A6,I1,M6,E8,P12);(A6,I1,M6,E8,P13);(A6,I1,M6,E8,P14);(A6,I1,M6,E8,P15);(A6,I1,M6,E8,P16);(A6,I1,M6,E8,P17);(A6,I1,M6,E8,P18);(A6,I1,M6,E8,P19);(A6,I1,M6,E8,P20);(A6,I1,M6,E9,P1);(A6,I1,M6,E9,P2);(A6,I1,M6,E9,P3);(A6,I1,M6,E9,P4);(A6,I1,M6,E9,P5);(A6,I1,M6,E9,P6);(A6,I1,M6,E9,P7);(A6,1 1,M6,E9,P8);(A6,I1,M6,E9,P9);(A6,I1,M6,E9,P10);(A6,I1,M6,E9,P11);(A6,I1,M6,E9,P12);(A6,I1,M6,E9,P13);(A6,I1,M6,E9,P14);(A6,I1,M6,E9,P15);(A6,I1,M6,E9,P16);(A6,I1,M6,E9,P17);(A6,I1,M6,E9,P18);(A6,I1,M6,E9,P19);(A6,I1,M6,E9,P20);(A6,I2,M1,E1,P1);(A6,I2,M1,E1,P2);(A6,I2,M1,E1,P3);(A6,I2,M1,E1,P4);(A6,I2,M1,E1,P5);(A6,I2,M1,E1,P6);(A6,I2,M1,E1,P7);(A6,I2,M1,E1,P8);(A6,I2,M1,E1,P9);(A6,I2,M1,E1,P10);(A6,I2,M1,E1,P11);(A6,I2,M1,E1,P12);(A6,I2,M1,E1,P13);(A6,I2,M1,E1,P14);(A6,I2,M1,E1,P15);(A6,I2,M1,E1,P16);(A6,I2,M1,E1,P17);(A6,I2,M1,E1,P18);(A6,I2,M1,E1,P19);(A6,I2,M1,E1,P20);(A6,I2,M1,E2,P1);(A6,I2,M1,E2,P2);(A6,I2,M1,E2,P3);(A6,I2,M1,E2,P4);(A6,I2,M1,E2,P5);(A6,I2,M1,E2,P6);(A6,I2,M1,E2,P7);(A6,I2,M1,E2,P8);(A6,I2,M1,E2,P9);(A6,I2,M1,E2,P10);(A6,I2,M1,E2,P11);(A6,I2,M1,E2,P12);(A6,I2,M1,E2,P13);(A6,I2,M1,E2,P14);(A6,I2,M1,E2,P15);(A6,I2,M1,E2,P16);(A6,I2,M1,E2,P17);(A6,I2,M1,E2,P18);(A6,I2,M1,E2,P19);(A6,I2,M1,E2,P20);(A6,I2,M1,E3,P1);(A6,I2,M1,E3,P2);(A6,I2,M1,E3,P3);(A6,I2,M1,E3,P4);(A6,I2,M1,E3,P5);(A6,I2,M1,E3,P6);(A6,I2,M1,E3,P7);(A6,I2,M1,E3,P8);(A6,I2,M1,E3,P9);(A6,I2,M1,E3,P10);(A6,I2,M1,E3,P11);(A6,I2,M1,E3,P12);(A6,I2,M1,E3,P13);(A6,I2,M1,E3,P14);(A6,I2,M1,E3,P15);(A6,I2,M1,E3,P16);(A6,I2,M1,E3,P17);(A6,I2,M1,E3,P18);(A6,I2,M1,E3,P19);(A6,I2,M1,E3,P20);(A6,I2,M1,E4,P1);(A6,I2,M1,E4,P2);(A6,I2,M1,E4,P3);(A6,I2,M1,E4,P4);(A6,I2,M1,E4,P5);(A6,I2,M1,E4,P6);(A6,I2,M1,E4,P7);(A6,I2,M1,E4,P8);(A6,I2,M1,E4,P9);(A6,I2,M1,E4,P10);(A6,I2,M1,E4,P11);(A6,I2,M1,E4,P12);(A6,I2,M1,E4,P13);(A6,I2,M1,E4,P14);(A6,I2,M1,E4,P15);(A6,I2,M1,E4,P16);(A6,I2,M1,E4,P17);(A6,I2,M1,E4,P18);(A6,I2,M1,E4,P19);(A6,I2,M1,E4,P20);(A6,I2,M1,E5,P1);(A6,I2,M1,E5,P2);(A6,I2,M1,E5,P3);(A6,I2,M1,E5,P4);(A6,I2,M1,E5,P5);(A6,I2,M1,E5,P6);(A6,I2,M1,E5,P7);(A6,I2,M1,E5,P8);(A6,I2,M1,E5,P9);(A6,I2,M1,E5,P10);(A6,I2,M1,E5,P11);(A6,I2,M1,E5,P12);(A6,I2,M1,E5,P13);(A6,I2,M1,E5,P14);(A6,I2,M1,E5,P15);(A6,I2,M1,E5,P16);(A6,I2,M1,E5,P17);(A6,I2,M1,E5,P18);(A6,I2,M1,E5,P19);(A6,I2,M1,E5,P20);(A6,I2,M1,E6,P1);(A6,I2,M1,E6,P2);(A6,I2,M1,E6,P3);(A6,I2,M1,E6,P4);(A6,I2,M1,E6,P5);(A6,I2,M1,E6,P6);(A6,I2,M1,E6,P7);(A6,I2,M1,E6,P8);(A6,I2,M1,E6,P9);(A6,I2,M1,E6,P10);(A6,I2,M1,E6,P11);(A6,I2,M1,E6,P12);(A6,I2,M1,E6,P13);(A6,I2,M1,E6,P14);(A6,I2,M1,E6,P15);(A6,I2,M1,E6,P16);(A6,I2,M1,E6,P17);(A6,I2,M1,E6,P18);(A6,I2,M1,E6,P19);(A6,I2,M1,E6,P20);(A6,I2,M1,E7,P1);(A6,I2,M1,E7,P2);(A6,I2,M1,E7,P3);(A6,I2,M1,E7,P4);(A6,I2,M1,E7,P5);(A6,I2,M1,E7,P6);(A6,I2,M1,E7,P7);(A6,I2,M1,E7,P8);(A6,I2,M1,E7,P9);(A6,I2,M1,E7,P10);(A6,I2,M1,E7,P11);(A6,I2,M1,E7,P12);(A6,I2,M1,E7,P13);(A6,I2,M1,E7,P14);(A6,I2,M1,E7,P15);(A6,I2,M1,E7,P16);(A6,I2,M1,E7,P17);(A6,I2,M1,E7,P18);(A6,I2,M1,E7,P19);(A6,I2,M1,E7,P20);(A6,I2,M1,E8,P1);(A6,I2,M1,E8,P2);(A6,I2,M1,E8,P3);(A6,I2,M1,E8,P4);(A6,I2,M1,E8,P5);(A6,I2,M1,E8,P6);(A6,I2,M1,E8,P7);(A6,I2,M1,E8,P8);(A6,I2,M1,E8,P9);(A6,I2,M1,E8,P10);(A6,I2,M1,E8,P11);(A6,I2,M1,E8,P12);(A6,I2,M1,E8,P13);(A6,I2,M1,E8,P14);(A6,I2,M1,E8,P15);(A6,I2,M1,E8,P16);(A6,I2,M1,E8,P17);(A6,I2,M1,E8,P18);(A6,I2,M1,E8,P19);(A6,I2,M1,E8,P20);(A6,I2,M1,E9,P1);(A6,I2,M1,E9,P2);(A6,I2,M1,E9,P3);(A6,I2,M1,E9,P4);(A6,I2,M1,E9,P5);(A6,I2,M1,E9,P6);(A6,I2,M1,E9,P7);(A6,I2,M1,E9,P8);(A6,I2,M1,E9,P9);(A6,I2,M1,E9,P10);(A6,I2,M1,E9,P11);(A6,I2,M1,E9,P12);(A6,I2,M1,E9,P13);(A6,I2,M1,E9,P14);(A6,I2,M1,E9,P15);(A6,I2,M1,E9,P16);(A6,I2,M1,E9,P17);(A6,I2,M1,E9,P18);(A6,I2,M1,E9,P19);(A6,I2,M1,E9,P20);(A6,I2,M2,E1,P1);(A6,I2,M2,E1,P2);(A6,I2,M2,E1,P3);(A6,I2,M2,E1,P4);(A6,I2,M2,E1,P5);(A6,I2,M2,E1,P6);(A6,I2,M2,E1,P7);(A6,I2,M2,E1,P8);(A6,I2,M2,E1,P9);(A6,I2,M2,E1,P10);(A6,I2,M2,E1,P11);(A6,I2,M2,E1,P12);(A6,I2,M2,E1,P13);(A6,I2,M2,E1,P14);(A6,I2,M2,E1,P15);(A6,I2,M2,E1,P16);(A6,I2,M2,E1,P17);(A6,I2,M2,E1,P18);(A6,I2,M2,E1,P19);(A6,I2,M2,E1,P20);(A6,I2,M2,E2,P1);(A6,I2,M2,E2,P2);(A6,I2,M2,E2,P3);(A6,I2,M2,E2,P4);(A6,I2,M2,E2,P5);(A6,I2,M2,E2,P6);(A6,I2,M2,E2,P7);(A6,I2,M2,E2,P8);(A6,I2,M2,E2,P9);(A6,I2,M2,E2,P10);(A6,I2,M2,E2,P11);(A6,I2,M2,E2,P12);(A6,I2,M2,E2,P13);(A6,I2,M2,E2,P14);(A6,I2,M2,E2,P15);(A6,I2,M2,E2,P16);(A6,I2,M2,E2,P17);(A6,I2,M2,E2,P18);(A6,I2,M2,E2,P19);(A6,I2,M2,E2,P20);(A6,I2,M2,E3,P1);(A6,I2,M2,E3,P2);(A6,I2,M2,E3,P3);(A6,I2,M2,E3,P4);(A6,I2,M2,E3,P5);(A6,I2,M2,E3,P6);(A6,I2,M2,E3,P7);(A6,I2,M2,E3,P8);(A6,I2,M2,E3,P9);(A6,I2,M2,E3,P10);(A6,I2,M2,E3,P11);(A6,I2,M2,E3,P12);(A6,I2,M2,E3,P13);(A6,I2,M2,E3,P14);(A6,I2,M2,E3,P15);(A6,I2,M2,E3,P16);(A6,I2,M2,E3,P17);(A6,I2,M2,E3,P18);(A6,I2,M2,E3,P19);(A6,I2,M2,E3,P20);(A6,I2,M2,E4,P1);(A6,I2,M2,E4,P2);(A6,I2,M2,E4,P3);(A6,I2,M2,E4,P4);(A6,I2,M2,E4,P5);(A6,I2,M2,E4,P6);(A6,I2,M2,E4,P7);(A6,I2,M2,E4,P8);(A6,I2,M2,E4,P9);(A6,I2,M2,E4,P10);(A6,I2,M2,E4,P11);(A6,I2,M2,E4,P12);(A6,I2,M2,E4,P13);(A6,I2,M2,E4,P14);(A6,I2,M2,E4,P15);(A6,I2,M2,E4,P16);(A6,I2,M2,E4,P17);(A6,I2,M2,E4,P18);(A6,I2,M2,E4,P19);(A6,I2,M2,E4,P20);(A6,I2,M2,E5,P1);(A6,I2,M2,E5,P2);(A6,I2,M2,E5,P3);(A6,I2,M2,E5,P4);(A6,I2,M2,E5,P5);(A6,I2,M2,E5,P6);(A6,I2,M2,E5,P7);(A6,I2,M2,E5,P8);(A6,I2,M2,E5,P9);(A6,I2,M2,E5,P10);(A6,I2,M2,E5,P11);(A6,I2,M2,E5,P12);(A6,I2,M2,E5,P13);(A6,I2,M2,E5,P14);(A6,I2,M2,E5,P15);(A6,I2,M2,E5,P16);(A6,I2,M2,E5,P17);(A6,I2,M2,E5,P18);(A6,I2,M2,E5,P19);(A6,I2,M2,E5,P20);(A6,I2,M2,E6,P1);(A6,I2,M2,E6,P2);(A6,I2,M2,E6,P3);(A6,I2,M2,E6,P4);(A6,I2,M2,E6,P5);(A6,I2,M2,E6,P6);(A6,I2,M2,E6,P7);(A6,I2,M2,E6,P8);(A6,I2,M2,E6,P9);(A6,I2,M2,E6,P10);(A6,I2,M2,E6,P11);(A6,I2,M2,E6,P12);(A6,I2,M2,E6,P13);(A6,I2,M2,E6,P14);(A6,I2,M2,E6,P15);(A6,I2,M2,E6,P16);(A6,I2,M2,E6,P17);(A6,I2,M2,E6,P18);(A6,I2,M2,E6,P19);(A6,I2,M2,E6,P20);(A6,I2,M2,E7,P1);(A6,I2,M2,E7,P2);(A6,I2,M2,E7,P3);(A6,I2,M2,E7,P4);(A6,I2,M2,E7,P5);(A6,I2,M2,E7,P6);(A6,I2,M2,E7,P7);(A6,I2,M2,E7,P8);(A6,I2,M2,E7,P9);(A6,I2,M2,E7,P10);(A6,I2,M2,E7,P11);(A6,I2,M2,E7,P12);(A6,I2,M2,E7,P13);(A6,I2,M2,E7,P14);(A6,I2,M2,E7,P15);(A6,I2,M2,E7,P16);(A6,I2,M2,E7,P17);(A6,I2,M2,E7,P18);(A6,I2,M2,E7,P19);(A6,I2,M2,E7,P20);(A6,I2,M2,E8,P1);(A6,I2,M2,E8,P2);(A6,I2,M2,E8,P3);(A6,I2,M2,E8,P4);(A6,I2,M2,E8,P5);(A6,I2,M2,E8,P6);(A6,I2,M2,E8,P7);(A6,I2,M2,E8,P8);(A6,I2,M2,E8,P9);(A6,I2,M2,E8,P10);(A6,I2,M2,E8,P11);(A6,I2,M2,E8,P12);(A6,I2,M2,E8,P13);(A6,I2,M2,E8,P14);(A6,I2,M2,E8,P15);(A6,I2,M2,E8,P16);(A6,I2,M2,E8,P17);(A6,I2,M2,E8,P18);(A6,I2,M2,E8,P19);(A6,I2,M2,E8,P20);(A6,I2,M2,E9,P1);(A6,I2,M2,E9,P2);(A6,I2,M2,E9,P3);(A6,I2,M2,E9,P4);(A6,I2,M2,E9,P5);(A6,I2,M2,E9,P6);(A6,I2,M2,E9,P7);(A6,I2,M2,E9,P8);(A6,I2,M2,E9,P9);(A6,I2,M2,E9,P10);(A6,I2,M2,E9,P11);(A6,I2,M2,E9,P12);(A6,I2,M2,E9,P13);(A6,I2,M2,E9,P14);(A6,I2,M2,E9,P15);(A6,I2,M2,E9,P16);(A6,I2,M2,E9,P17);(A6,I2,M2,E9,P18);(A6,I2,M2,E9,P19);(A6,I2,M2,E9,P20);(A6,I2,M3,E1,P1);(A6,I2,M3,E1,P2);(A6,I2,M3,E1,P3);(A6,I2,M3,E1,P4);(A6,I2,M3,E1,P5);(A6,I2,M3,E1,P6);(A6,I2,M3,E1,P7);(A6,I2,M3,E1,P8);(A6,I2,M3,E1,P9);(A6,I2,M3,E1,P10);(A6,I2,M3,E1,P11);(A6,I2,M3,E1,P12);(A6,I2,M3,E1,P13);(A6,I2,M3,E1,P14);(A6,I2,M3,E1,P15);(A6,I2,M3,E1,P16);(A6,I2,M3,E1,P17);(A6,I2,M3,E1,P18);(A6,I2,M3,E1,P19);(A6,I2,M3,E1,P20);(A6,I2,M3,E2,P1);(A6,I2,M3,E2,P2);(A6,I2,M3,E2,P3);(A6,I2,M3,E2,P4);(A6,I2,M3,E2,P5);(A6,I2,M3,E2,P6);(A6,I2,M3,E2,P7);(A6,I2,M3,E2,P8);(A6,I2,M3,E2,P9);(A6,I2,M3,E2,P10);(A6,I2,M3,E2,P11);(A6,I2,M3,E2,P12);(A6,I2,M3,E2,P13);(A6,I2,M3,E2,P14);(A6,I2,M3,E2,P15);(A6,I2,M3,E2,P16);(A6,I2,M3,E2,P17);(A6,I2,M3,E2,P18);(A6,I2,M3,E2,P19);(A6,I2,M3,E2,P20);(A6,I2,M3,E3,P1);(A6,I2,M3,E3,P2);(A6,I2,M3,E3,P3);(A6,I2,M3,E3,P4);(A6,I2,M3,E3,P5);(A6,I2,M3,E3,P6);(A6,I2,M3,E3,P7);(A6,I2,M3,E3,P8);(A6,I2,M3,E3,P9);(A6,I2,M3,E3,P10);(A6,I2,M3,E3,P11);(A6,I2,M3,E3,P12);(A6,I2,M3,E3,P13);(A6,I2,M3,E3,P14);(A6,I2,M3,E3,P15);(A6,I2,M3,E3,P16);(A6,I2,M3,E3,P17);(A6,I2,M3,E3,P18);(A6,I2,M3,E3,P19);(A6,I2,M3,E3,P20);(A6,I2,M3,E4,P1);(A6,I2,M3,E4,P2);(A6,I2,M3,E4,P3);(A6,I2,M3,E4,P4);(A6,I2,M3,E4,P5);(A6,I2,M3,E4,P6);(A6,I2,M3,E4,P7);(A6,I2,M3,E4,P8);(A6,I2,M3,E4,P9);(A6,I2,M3,E4,P10);(A6,I2,M3,E4,P11);(A6,I2,M3,E4,P12);(A6,I2,M3,E4,P13);(A6,I2,M3,E4,P14);(A6,I2,M3,E4,P15);(A6,I2,M3,E4,P16);(A6,I2,M3,E4,P17);(A6,I2,M3,E4,P18);(A6,I2,M3,E4,P19);(A6,I2,M3,E4,P20);(A6,I2,M3,E5,P1);(A6,I2,M3,E5,P2);(A6,I2,M3,E5,P3);(A6,I2,M3,E5,P4);(A6,I2,M3,E5,P5);(A6,I2,M3,E5,P6);(A6,I2,M3,E5,P7);(A6,I2,M3,E5,P8);(A6,I2,M3,E5,P9);(A6,I2,M3,E5,P10);(A6,I2,M3,E5,P11);(A6,I2,M3,E5,P12);(A6,I2,M3,E5,P13);(A6,I2,M3,E5,P14);(A6,I2,M3,E5,P15);(A6,I2,M3,E5,P16);(A6,I2,M3,E5,P17);(A6,I2,M3,E5,P18);(A6,I2,M3,E5,P19);(A6,I2,M3,E5,P20);(A6,I2,M3,E6,P1);(A6,I2,M3,E6,P2);(A6,I2,M3,E6,P3);(A6,I2,M3,E6,P4);(A6,I2,M3,E6,P5);(A6,I2,M3,E6,P6);(A6,I2,M3,E6,P7);(A6,I2,M3,E6,P8);(A6,I2,M3,E6,P9);(A6,I2,M3,E6,P10);(A6,I2,M3,E6,P11);(A6,I2,M3,E6,P12);(A6,I2,M3,E6,P13);(A6,I2,M3,E6,P14);(A6,I2,M3,E6,P15);(A6,I2,M3,E6,P16);(A6,I2,M3,E6,P17);(A6,I2,M3,E6,P18);(A6,I2,M3,E6,P19);(A6,I2,M3,E6,P20);(A6,I2,M3,E7,P1);(A6,I2,M3,E7,P2);(A6,I2,M3,E7,P3);(A6,I2,M3,E7,P4);(A6,I2,M3,E7,P5);(A6,I2,M3,E7,P6);(A6,I2,M3,E7,P7);(A6,I2,M3,E7,P8);(A6,I2,M3,E7,P9);(A6,I2,M3,E7,P10);(A6,I2,M3,E7,P11);(A6,I2,M3,E7,P12);(A6,I2,M3,E7,P13);(A6,I2,M3,E7,P14);(A6,I2,M3,E7,P15);(A6,I2,M3,E7,P16);(A6,I2,M3,E7,P17);(A6,I2,M3,E7,P18);(A6,I2,M3,E7,P19);(A6,I2,M3,E7,P20);(A6,I2,M3,E8,P1);(A6,I2,M3,E8,P2);(A6,I2,M3,E8,P3);(A6,I2,M3,E8,P4);(A6,I2,M3,E8,P5);(A6,I2,M3,E8,P6);(A6,I2,M3,E8,P7);(A6,I2,M3,E8,P8);(A6,I2,M3,E8,P9);(A6,I2,M3,E8,P10);(A6,I2,M3,E8,P11);(A6,I2,M3,E8,P12);(A6,I2,M3,E8,P13);(A6,I2,M3,E8,P14);(A6,I2,M3,E8,P15);(A6,I2,M3,E8,P16);(A6,I2,M3,E8,P17);(A6,I2,M3,E8,P18);(A6,I2,M3,E8,P19);(A6,I2,M3,E8,P20);(A6,I2,M3,E9,P1);(A6,I2,M3,E9,P2);(A6,I2,M3,E9,P3);(A6,I2,M3,E9,P4);(A6,I2,M3,E9,P5);(A6,I2,M3,E9,P6);(A6,I2,M3,E9,P7);(A6,I2,M3,E9,P8);(A6,I2,M3,E9,P9);(A6,I2,M3,E9,P10);(A6,I2,M3,E9,P11);(A6,I2,M3,E9,P12);(A6,I2,M3,E9,P13);(A6,I2,M3,E9,P14);(A6,I2,M3,E9,P15);(A6,I2,M3,E9,P16);(A6,I2,M3,E9,P17);(A6,I2,M3,E9,P18);(A6,I2,M3,E9,P19);(A6,I2,M3,E9,P20);(A6,I2,M4,E1,P1);(A6,I2,M4,E1,P2);(A6,I2,M4,E1,P3);(A6,I2,M4,E1,P4);(A6,I2,M4,E1,P5);(A6,I2,M4,E1,P6);(A6,I2,M4,E1,P7);(A6,I2,M4,E1,P8);(A6,I2,M4,E1,P9);(A6,I2,M4,E1,P10);(A6,I2,M4,E1,P11);(A6,I2,M4,E1,P12);(A6,I2,M4,E1,P13);(A6,I2,M4,E1,P14);(A6,I2,M4,E1,P15);(A6,I2,M4,E1,P16);(A6,I2,M4,E1,P17);(A6,I2,M4,E1,P18);(A6,I2,M4,E1,P19);(A6,I2,M4,E1,P20);(A6,I2,M4,E2,P1);(A6,I2,M4,E2,P2);(A6,I2,M4,E2,P3);(A6,I2,M4,E2,P4);(A6,I2,M4,E2,P5);(A6,I2,M4,E2,P6);(A6,I2,M4,E2,P7);(A6,I2,M4,E2,P8);(A6,I2,M4,E2,P9);(A6,I2,M4,E2,P10);(A6,I2,M4,E2,P11);(A6,I2,M4,E2,P12);(A6,I2,M4,E2,P13);(A6,I2,M4,E2,P14);(A6,I2,M4,E2,P15);(A6,I2,M4,E2,P16);(A6,I2,M4,E2,P17);(A6,I2,M4,E2,P18);(A6,I2,M4,E2,P19);(A6,I2,M4,E2,P20);(A6,I2,M4,E3,P1);(A6,I2,M4,E3,P2);(A6,I2,M4,E3,P3);(A6,I2,M4,E3,P4);(A6,I2,M4,E3,P5);(A6,I2,M4,E3,P6);(A6,I2,M4,E3,P7);(A6,I2,M4,E3,P8);(A6,I2,M4,E3,P9);(A6,I2,M4,E3,P10);(A6,I2,M4,E3,P11);(A6,I2,M4,E3,P12);(A6,I2,M4,E3,P13);(A6,I2,M4,E3,P14);(A6,I2,M4,E3,P15);(A6,I2,M4,E3,P16);(A6,I2,M4,E3,P17);(A6,I2,M4,E3,P18);(A6,I2,M4,E3,P19);(A6,I2,M4,E3,P20);(A6,I2,M4,E4,P1);(A6,I2,M4,E4,P2);(A6,I2,M4,E4,P3);(A6,I2,M4,E4,P4);(A6,I2,M4,E4,P5);(A6,I2,M4,E4,P6);(A6,I2,M4,E4,P7);(A6,I2,M4,E4,P8);(A6,I2,M4,E4,P9);(A6,I2,M4,E4,P10);(A6,I2,M4,E4,P11);(A6,I2,M4,E4,P12);(A6,I2,M4,E4,P13);(A6,I2,M4,E4,P14);(A6,I2,M4,E4,P15);(A6,I2,M4,E4,P16);(A6,I2,M4,E4,P17);(A6,I2,M4,E4,P18);(A6,I2,M4,E4,P19);(A6,I2,M4,E4,P20);(A6,I2,M4,E5,P1);(A6,I2,M4,E5,P2);(A6,I2,M4,E5,P3);(A6,I2,M4,E5,P4);(A6,I2,M4,E5,P5);(A6,I2,M4,E5,P6);(A6,I2,M4,E5,P7);(A6,I2,M4,E5,P8);(A6,I2,M4,E5,P9);(A6,I2,M4,E5,P10);(A6,I2,M4,E5,P11);(A6,I2,M4,E5,P12);(A6,I2,M4,E5,P13);(A6,I2,M4,E5,P14);(A6,I2,M4,E5,P15);(A6,I2,M4,E5,P16);(A6,I2,M4,E5,P17);(A6,I2,M4,E5,P18);(A6,I2,M4,E5,P19);(A6,I2,M4,E5,P20);(A6,I2,M4,E6,P1);(A6,I2,M4,E6,P2);(A6,I2,M4,E6,P3);(A6,I2,M4,E6,P4);(A6,I2,M4,E6,P5);(A6,I2,M4,E6,P6);(A6,I2,M4,E6,P7);(A6,I2,M4,E6,P8);(A6,I2,M4,E6,P9);(A6,I2,M4,E6,P10);(A6,I2,M4,E6,P11);(A6,I2,M4,E6,P12);(A6,I2,M4,E6,P13);(A6,I2,M4,E6,P14);(A6,I2,M4,E6,P15);(A6,I2,M4,E6,P16);(A6,I2,M4,E6,P17);(A6,I2,M4,E6,P18);(A6,I2,M4,E6,P19);(A6,I2,M4,E6,P20);(A6,I2,M4,E7,P1);(A6,I2,M4,E7,P2);(A6,I2,M4,E7,P3);(A6,I2,M4,E7,P4);(A6,I2,M4,E7,P5);(A6,I2,M4,E7,P6);(A6,I2,M4,E7,P7);(A6,I2,M4,E7,P8);(A6,I2,M4,E7,P9);(A6,I2,M4,E7,P10);(A6,I2,M4,E7,P11);(A6,I2,M4,E7,P12);(A6,I2,M4,E7,P13);(A6,I2,M4,E7,P14);(A6,I2,M4,E7,P15);(A6,I2,M4,E7,P16);(A6,I2,M4,E7,P17);(A6,I2,M4,E7,P18);(A6,I2,M4,E7,P19);(A6,I2,M4,E7,P20);(A6,I2,M4,E8,P1);(A6,I2,M4,E8,P2);(A6,I2,M4,E8,P3);(A6,I2,M4,E8,P4);(A6,I2,M4,E8,P5);(A6,I2,M4,E8,P6);(A6,I2,M4,E8,P7);(A6,I2,M4,E8,P8);(A6,I2,M4,E8,P9);(A6,I2,M4,E8,P10);(A6,I2,M4,E8,P11);(A6,I2,M4,E8,P12);(A6,I2,M4,E8,P13);(A6,I2,M4,E8,P14);(A6,I2,M4,E8,P15);(A6,I2,M4,E8,P16);(A6,I2,M4,E8,P17);(A6,I2,M4,E8,P18);(A6,I2,M4,E8,P19);(A6,I2,M4,E8,P20);(A6,I2,M4,E9,P1);(A6,I2,M4,E9,P2);(A6,I2,M4,E9,P3);(A6,I2,M4,E9,P4);(A6,I2,M4,E9,P5);(A6,I2,M4,E9,P6);(A6,I2,M4,E9,P7);(A6,I2,M4,E9,P8);(A6,I2,M4,E9,P9);(A6,I2,M4,E9,P10);(A6,I2,M4,E9,P11);(A6,I2,M4,E9,P12);(A6,I2,M4,E9,P13);(A6,I2,M4,E9,P14);(A6,I2,M4,E9,P15);(A6,I2,M4,E9,P16);(A6,I2,M4,E9,P17);(A6,I2,M4,E9,P18);(A6,I2,M4,E9,P19);(A6,I2,M4,E9,P20);(A6,I2,M5,E1,P1);(A6,I2,M5,E1,P2);(A6,I2,M5,E1,P3);(A6,I2,M5,E1,P4);(A6,I2,M5,E1,P5);(A6,I2,M5,E1,P6);(A6,I2,M5,E1,P7);(A6,I2,M5,E1,P8);(A6,I2,M5,E1,P9);(A6,I2,M5,E1,P10);(A6,I2,M5,E1,P11);(A6,I2,M5,E1,P12);(A6,I2,M5,E1,P13);(A6,I2,M5,E1,P14);(A6,I2,M5,E1,P15);(A6,I2,M5,E1,P16);(A6,I2,M5,E1,P17);(A6,I2,M5,E1,P18);(A6,I2,M5,E1,P19);(A6,I2,M5,E1,P20);(A6,I2,M5,E2,P1);(A6,I2,M5,E2,P2);(A6,I2,M5,E2,P3);(A6,I2,M5,E2,P4);(A6,I2,M5,E2,P5);(A6,I2,M5,E2,P6);(A6,I2,M5,E2,P7);(A6,I2,M5,E2,P8);(A6,I2,M5,E2,P9);(A6,I2,M5,E2,P10);(A6,I2,M5,E2,P11);(A6,I2,M5,E2,P12);(A6,I2,M5,E2,P13);(A6,I2,M5,E2,P14);(A6,I2,M5,E2,P15);(A6,I2,M5,E2,P16);(A6,I2,M5,E2,P17);(A6,I2,M5,E2,P18);(A6,I2,M5,E2,P19);(A6,I2,M5,E2,P20);(A6,I2,M5,E3,P1);(A6,I2,M5,E3,P2);(A6,I2,M5,E3,P3);(A6,I2,M5,E3,P4);(A6,I2,M5,E3,P5);(A6,I2,M5,E3,P6);(A6,I2,M5,E3,P7);(A6,I2,M5,E3,P8);(A6,I2,M5,E3,P9);(A6,I2,M5,E3,P10);(A6,I2,M5,E3,P11);(A6,I2,M5,E3,P12);(A6,I2,M5,E3,P13);(A6,I2,M5,E3,P14);(A6,I2,M5,E3,P15);(A6,I2,M5,E3,P16);(A6,I2,M5,E3,P17);(A6,I2,M5,E3,P18);(A6,I2,M5,E3,P19);(A6,I2,M5,E3,P20);(A6,I2,M5,E4,P1);(A6,I2,M5,E4,P2);(A6,I2,M5,E4,P3);(A6,I2,M5,E4,P4);(A6,I2,M5,E4,P5);(A6,I2,M5,E4,P6);(A6,I2,M5,E4,P7);(A6,I2,M5,E4,P8);(A6,I2,M5,E4,P9);(A6,I2,M5,E4,P10);(A6,I2,M5,E4,P11);(A6,I2,M5,E4,P12);(A6,I2,M5,E4,P13);(A6,I2,M5,E4,P14);(A6,I2,M5,E4,P15);(A6,I2,M5,E4,P16);(A6,I2,M5,E4,P17);(A6,I2,M5,E4,P18);(A6,I2,M5,E4,P19);(A6,I2,M5,E4,P20);(A6,I2,M5,E5,P1);(A6,I2,M5,E5,P2);(A6,I2,M5,E5,P3);(A6,I2,M5,E5,P4);(A6,I2,M5,E5,P5);(A6,I2,M5,E5,P6);(A6,I2,M5,E5,P7);(A6,I2,M5,E5,P8);(A6,I2,M5,E5,P9);(A6,I2,M5,E5,P10);(A6,I2,M5,E5,P11);(A6,I2,M5,E5,P12);(A6,I2,M5,E5,P13);(A6,I2,M5,E5,P14);(A6,I2,M5,E5,P15);(A6,I2,M5,E5,P16);(A6,I2,M5,E5,P17);(A6,I2,M5,E5,P18);(A6,I2,M5,E5,P19);(A6,I2,M5,E5,P20);(A6,I2,M5,E6,P1);(A6,I2,M5,E6,P2);(A6,I2,M5,E6,P3);(A6,I2,M5,E6,P4);(A6,I2,M5,E6,P5);(A6,I2,M5,E6,P6);(A6,I2,M5,E6,P7);(A6,I2,M5,E6,P8);(A6,I2,M5,E6,P9);(A6,I2,M5,E6,P10);(A6,I2,M5,E6,P11);(A6,I2,M5,E6,P12);(A6,I2,M5,E6,P13);(A6,I2,M5,E6,P14);(A6,I2,M5,E6,P15);(A6,I2,M5,E6,P16);(A6,I2,M5,E6,P17);(A6,I2,M5,E6,P18);(A6,I2,M5,E6,P19);(A6,I2,M5,E6,P20);(A6,I2,M5,E7,P1);(A6,I2,M5,E7,P2);(A6,I2,M5,E7,P3);(A6,I2,M5,E 7,P4);(A6,I2,M5,E7,P5);(A6,I2,M5,E7,P6);(A6,I2,M5,E7,P7);(A6,I2,M5,E7,P8);(A6,I2,M5,E7,P9);(A6,I2,M5,E7,P10);(A6,I2,M5,E7,P11);(A6,I2,M5,E7,P12);(A6,I2,M5,E7,P13);(A6,I2,M5,E7,P14);(A6,I2,M5,E7,P15);(A6,I2,M5,E7,P16);(A6,I2,M5,E7,P17);(A6,I2,M5,E7,P18);(A6,I2,M5,E7,P19);(A6,I2,M5,E7,P20);(A6,I2,M5,E8,P1);(A6,I2,M5,E8,P2);(A6,I2,M5,E8,P3);(A6,I2,M5,E8,P4);(A6,I2,M5,E8,P5);(A6,I2,M5,E8,P6);(A6,I2,M5,E8,P7);(A6,I2,M5,E8,P8);(A6,I2,M5,E8,P9);(A6,I2,M5,E8,P10);(A6,I2,M5,E8,P11);(A6,I2,M5,E8,P12);(A6,I2,M5,E8,P13);(A6,I2,M5,E8,P14);(A6,I2,M5,E8,P15);(A6,I2,M5,E8,P16);(A6,I2,M5,E8,P17);(A6,I2,M5,E8,P18);(A6,I2,M5,E8,P19);(A6,I2,M5,E8,P20);(A6,I2,M5,E9,P1);(A6,I2,M5,E9,P2);(A6,I2,M5,E9,P3);(A6,I2,M5,E9,P4);(A6,I2,M5,E9,P5);(A6,I2,M5,E9,P6);(A6,I2,M5,E9,P7);(A6,I2,M5,E9,P8);(A6,I2,M5,E9,P9);(A6,I2,M5,E9,P10);(A6,I2,M5,E9,P11);(A6,I2,M5,E9,P12);(A6,I2,M5,E9,P13);(A6,I2,M5,E9,P14);(A6,I2,M5,E9,P15);(A6,I2,M5,E9,P16);(A6,I2,M5,E9,P17);(A6,I2,M5,E9,P18);(A6,I2,M5,E9,P19);(A6,I2,M5,E9,P20);(A6,I2,M6,E1,P1);(A6,I2,M6,E1,P2);(A6,I2,M6,E1,P3);(A6,I2,M6,E1,P4);(A6,I2,M6,E1,P5);(A6,I2,M6,E1,P6);(A6,I2,M6,E1,P7);(A6,I2,M6,E1,P8);(A6,I2,M6,E1,P9);(A6,I2,M6,E1,P10);(A6,I2,M6,E1,P11);(A6,I2,M6,E1,P12);(A6,I2,M6,E1,P13);(A6,I2,M6,E1,P14);(A6,I2,M6,E1,P15);(A6,I2,M6,E1,P16);(A6,I2,M6,E1,P17);(A6,I2,M6,E1,P18);(A6,I2,M6,E1,P19);(A6,I2,M6,E1,P20);(A6,I2,M6,E2,P1);(A6,I2,M6,E2,P2);(A6,I2,M6,E2,P3);(A6,I2,M6,E2,P4);(A6,I2,M6,E2,P5);(A6,I2,M6,E2,P6);(A6,I2,M6,E2,P7);(A6,I2,M6,E2,P8);(A6,I2,M6,E2,P9);(A6,I2,M6,E2,P10);(A6,I2,M6,E2,P11);(A6,I2,M6,E2,P12);(A6,I2,M6,E2,P13);(A6,I2,M6,E2,P14);(A6,I2,M6,E2,P15);(A6,I2,M6,E2,P16);(A6,I2,M6,E2,P17);(A6,I2,M6,E2,P18);(A6,I2,M6,E2,P19);(A6,I2,M6,E2,P20);(A6,I2,M6,E3,P1);(A6,I2,M6,E3,P2);(A6,I2,M6,E3,P3);(A6,I2,M6,E3,P4);(A6,I2,M6,E3,P5);(A6,I2,M6,E3,P6);(A6,I2,M6,E3,P7);(A6,I2,M6,E3,P8);(A6,I2,M6,E3,P9);(A6,I2,M6,E3,P10);(A6,I2,M6,E3,P11);(A6,I2,M6,E3,P12);(A6,I2,M6,E3,P13);(A6,I2,M6,E3,P14);(A6,I2,M6,E3,P15);(A6,I2,M6,E3,P16);(A6,I2,M6,E3,P17);(A6,I2,M6,E3,P18);(A6,I2,M6,E3,P19);(A6,I2,M6,E3,P20);(A6,I2,M6,E4,P1);(A6,I2,M6,E4,P2);(A6,I2,M6,E4,P3);(A6,I2,M6,E4,P4);(A6,I2,M6,E4,P5);(A6,I2,M6,E4,P6);(A6,I2,M6,E4,P7);(A6,I2,M6,E4,P8);(A6,I2,M6,E4,P9);(A6,I2,M6,E4,P10);(A6,I2,M6,E4,P11);(A6,I2,M6,E4,P12);(A6,I2,M6,E4,P13);(A6,I2,M6,E4,P14);(A6,I2,M6,E4,P15);(A6,I2,M6,E4,P16);(A6,I2,M6,E4,P17);(A6,I2,M6,E4,P18);(A6,I2,M6,E4,P19);(A6,I2,M6,E4,P20);(A6,I2,M6,E5,P1);(A6,I2,M6,E5,P2);(A6,I2,M6,E5,P3);(A6,I2,M6,E5,P4);(A6,I2,M6,E5,P5);(A6,I2,M6,E5,P6);(A6,I2,M6,E5,P7);(A6,I2,M6,E5,P8);(A6,I2,M6,E5,P9);(A6,I2,M6,E5,P10);(A6,I2,M6,E5,P11);(A6,I2,M6,E5,P12);(A6,I2,M6,E5,P13);(A6,I2,M6,E5,P14);(A6,I2,M6,E5,P15);(A6,I2,M6,E5,P16);(A6,I2,M6,E5,P17);(A6,I2,M6,E5,P18);(A6,I2,M6,E5,P19);(A6,I2,M6,E5,P20);(A6,I2,M6,E6,P1);(A6,I2,M6,E6,P2);(A6,I2,M6,E6,P3);(A6,I2,M6,E6,P4);(A6,I2,M6,E6,P5);(A6,I2,M6,E6,P6);(A6,I2,M6,E6,P7);(A6,I2,M6,E6,P8);(A6,I2,M6,E6,P9);(A6,I2,M6,E6,P10);(A6,I2,M6,E6,P11);(A6,I2,M6,E6,P12);(A6,I2,M6,E6,P13);(A6,I2,M6,E6,P14);(A6,I2,M6,E6,P15);(A6,I2,M6,E6,P16);(A6,I2,M6,E6,P17);(A6,I2,M6,E6,P18);(A6,I2,M6,E6,P19);(A6,I2,M6,E6,P20);(A6,I2,M6,E7,P1);(A6,I2,M6,E7,P2);(A6,I2,M6,E7,P3);(A6,I2,M6,E7,P4);(A6,I2,M6,E7,P5);(A6,I2,M6,E7,P6);(A6,I2,M6,E7,P7);(A6,I2,M6,E7,P8);(A6,I2,M6,E7,P9);(A6,I2,M6,E7,P10);(A6,I2,M6,E7,P11);(A6,I2,M6,E7,P12);(A6,I2,M6,E7,P13);(A6,I2,M6,E7,P14);(A6,I2,M6,E7,P15);(A6,I2,M6,E7,P16);(A6,I2,M6,E7,P17);(A6,I2,M6,E7,P18);(A6,I2,M6,E7,P19);(A6,I2,M6,E7,P20);(A6,I2,M6,E8,P1);(A6,I2,M6,E8,P2);(A6,I2,M6,E8,P3);(A6,I2,M6,E8,P4);(A6,I2,M6,E8,P5);(A6,I2,M6,E8,P6);(A6,I2,M6,E8,P7);(A6,I2,M6,E8,P8);(A6,I2,M6,E8,P9);(A6,I2,M6,E8,P10);(A6,I2,M6,E8,P11);(A6,I2,M6,E8,P12);(A6,I2,M6,E8,P13);(A6,I2,M6,E8,P14);(A6,I2,M6,E8,P15);(A6,I2,M6,E8,P16);(A6,I2,M6,E8,P17);(A6,I2,M6,E8,P18);(A6,I2,M6,E8,P19);(A6,I2,M6,E8,P20);(A6,I2,M6,E9,P1);(A6,I2,M6,E9,P2);(A6,I2,M6,E9,P3);(A6,I2,M6,E9,P4);(A6,I2,M6,E9,P5);(A6,I2,M6,E9,P6);(A6,I2,M6,E9,P7);(A6,I2,M6,E9,P8);(A6,I2,M6,E9,P9);(A6,I2,M6,E9,P10);(A6,I2,M6,E9,P11);(A6,I2,M6,E9,P12);(A6,I2,M6,E9,P13);(A6,I2,M6,E9,P14);(A6,I2,M6,E9,P15);(A6,I2,M6,E9,P16);(A6,I2,M6,E9,P17);(A6,I2,M6,E9,P18);(A6,I2,M6,E9,P19);(A6,I2,M6,E9,P20);(A6,I3,M1,E1,P1);(A6,I3,M1,E1,P2);(A6,I3,M1,E1,P3);(A6,I3,M1,E1,P4);(A6,I3,M1,E1,P5);(A6,I3,M1,E1,P6);(A6,I3,M1,E1,P7);(A6,I3,M1,E1,P8);(A6,I3,M1,E1,P9);(A6,I3,M1,E1,P10);(A6,I3,M1,E1,P11);(A6,I3,M1,E1,P12);(A6,I3,M1,E1,P13);(A6,I3,M1,E1,P14);(A6,I3,M1,E1,P15);(A6,I3,M1,E1,P16);(A6,I3,M1,E1,P17);(A6,I3,M1,E1,P18);(A6,I3,M1,E1,P19);(A6,I3,M1,E1,P20);(A6,I3,M1,E2,P1);(A6,I3,M1,E2,P2);(A6,I3,M1,E2,P3);(A6,I3,M1,E2,P4);(A6,I3,M1,E2,P5);(A6,I3,M1,E2,P6);(A6,I3,M1,E2,P7);(A6,I3,M1,E2,P8);(A6,I3,M1,E2,P9);(A6,I3,M1,E2,P10);(A6,I3,M1,E2,P11);(A6,I3,M1,E2,P12);(A6,I3,M1,E2,P13);(A6,I3,M1,E2,P14);(A6,I3,M1,E2,P15);(A6,I3,M1,E2,P16);(A6,I3,M1,E2,P17);(A6,I3,M1,E2,P18);(A6,I3,M1,E2,P19);(A6,I3,M1,E2,P20);(A6,I3,M1,E3,P1);(A6,I3,M1,E3,P2);(A6,I3,M1,E3,P3);(A6,I3,M1,E3,P4);(A6,I3,M1,E3,P5);(A6,I3,M1,E3,P6);(A6,I3,M1,E3,P7);(A6,I3,M1,E3,P8);(A6,I3,M1,E3,P9);(A6,I3,M1,E3,P10);(A6,I3,M1,E3,P11);(A6,I3,M1,E3,P12);(A6,I3,M1,E3,P13);(A6,I3,M1,E3,P14);(A6,I3,M1,E3,P15);(A6,I3,M1,E3,P16);(A6,I3,M1,E3,P17);(A6,I3,M1,E3,P18);(A6,I3,M1,E3,P19);(A6,I3,M1,E3,P20);(A6,I3,M1,E4,P1);(A6,I3,M1,E4,P2);(A6,I3,M1,E4,P3);(A6,I3,M1,E4,P4);(A6,I3,M1,E4,P5);(A6,I3,M1,E4,P6);(A6,I3,M1,E4,P7);(A6,I3,M1,E4,P8);(A6,I3,M1,E4,P9);(A6,I3,M1,E4,P10);(A6,I3,M1,E4,P11);(A6,I3,M1,E4,P12);(A6,I3,M1,E4,P13);(A6,I3,M1,E4,P14);(A6,I3,M1,E4,P15);(A6,I3,M1,E4,P16);(A6,I3,M1,E4,P17);(A6,I3,M1,E4,P18);(A6,I3,M1,E4,P19);(A6,I3,M1,E4,P20);(A6,I3,M1,E5,P1);(A6,I3,M1,E5,P2);(A6,I3,M1,E5,P3);(A6,I3,M1,E5,P4);(A6,I3,M1,E5,P5);(A6,I3,M1,E5,P6);(A6,I3,M1,E5,P7);(A6,I3,M1,E5,P8);(A6,I3,M1,E5,P9);(A6,I3,M1,E5,P10);(A6,I3,M1,E5,P11);(A6,I3,M1,E5,P12);(A6,I3,M1,E5,P13);(A6,I3,M1,E5,P14);(A6,I3,M1,E5,P15);(A6,I3,M1,E5,P16);(A6,I3,M1,E5,P17);(A6,I3,M1,E5,P18);(A6,I3,M1,E5,P19);(A6,I3,M1,E5,P20);(A6,I3,M1,E6,P1);(A6,I3,M1,E6,P2);(A6,I3,M1,E6,P3);(A6,I3,M1,E6,P4);(A6,I3,M1,E6,P5);(A6,I3,M1,E6,P6);(A6,I3,M1,E6,P7);(A6,I3,M1,E6,P8);(A6,I3,M1,E6,P9);(A6,I3,M1,E6,P10);(A6,I3,M1,E6,P11);(A6,I3,M1,E6,P12);(A6,I3,M1,E6,P13);(A6,I3,M1,E6,P14);(A6,I3,M1,E6,P15);(A6,I3,M1,E6,P16);(A6,I3,M1,E6,P17);(A6,I3,M1,E6,P18);(A6,I3,M1,E6,P19);(A6,I3,M1,E6,P20);(A6,I3,M1,E7,P1);(A6,I3,M1,E7,P2);(A6,I3,M1,E7,P3);(A6,I3,M1,E7,P4);(A6,I3,M1,E7,P5);(A6,I3,M1,E7,P6);(A6,I3,M1,E7,P7);(A6,I3,M1,E7,P8);(A6,I3,M1,E7,P9);(A6,I3,M1,E7,P10);(A6,I3,M1,E7,P11);(A6,I3,M1,E7,P12);(A6,I3,M1,E7,P13);(A6,I3,M1,E7,P14);(A6,I3,M1,E7,P15);(A6,I3,M1,E7,P16);(A6,I3,M1,E7,P17);(A6,I3,M1,E7,P18);(A6,I3,M1,E7,P19);(A6,I3,M1,E7,P20);(A6,I3,M1,E8,P1);(A6,I3,M1,E8,P2);(A6,I3,M1,E8,P3);(A6,I3,M1,E8,P4);(A6,I3,M1,E8,P5);(A6,I3,M1,E8,P6);(A6,I3,M1,E8,P7);(A6,I3,M1,E8,P8);(A6,I3,M1,E8,P9);(A6,I3,M1,E8,P10);(A6,I3,M1,E8,P11);(A6,I3,M1,E8,P12);(A6,I3,M1,E8,P13);(A6,I3,M1,E8,P14);(A6,I3,M1,E8,P15);(A6,I3,M1,E8,P16);(A6,I3,M1,E8,P17);(A6,I3,M1,E8,P18);(A6,I3,M1,E8,P19);(A6,I3,M1,E8,P20);(A6,I3,M1,E9,P1);(A6,I3,M1,E9,P2);(A6,I3,M1,E9,P3);(A6,I3,M1,E9,P4);(A6,I3,M1,E9,P5);(A6,I3,M1,E9,P6);(A6,I3,M1,E9,P7);(A6,I3,M1,E9,P8);(A6,I3,M1,E9,P9);(A6,I3,M1,E9,P10);(A6,I3,M1,E9,P11);(A6,I3,M1,E9,P12);(A6,I3,M1,E9,P13);(A6,I3,M1,E9,P14);(A6,I3,M1,E9,P15);(A6,I3,M1,E9,P16);(A6,I3,M1,E9,P17);(A6,I3,M1,E9,P18);(A6,I3,M1,E9,P19);(A6,I3,M1,E9,P20);(A6,I3,M2,E1,P1);(A6,I3,M2,E1,P2);(A6,I3,M2,E1,P3);(A6,I3,M2,E1,P4);(A6,I3,M2,E1,P5);(A6,I3,M2,E1,P6);(A6,I3,M2,E1,P7);(A6,I3,M2,E1,P8);(A6,I3,M2,E1,P9);(A6,I3,M2,E1,P10);(A6,I3,M2,E1,P11);(A6,I3,M2,E1,P12);(A6,I3,M2,E1,P13);(A6,I3,M2,E1,P14);(A6,I3,M2,E1,P15);(A6,I3,M2,E1,P16);(A6,I3,M2,E1,P17);(A6,I3,M2,E1,P18);(A6,I3,M2,E1,P19);(A6,I3,M2,E1,P20);(A6,I3,M2,E2,P1);(A6,I3,M2,E2,P2);(A6,I3,M2,E2,P3);(A6,I3,M2,E2,P4);(A6,I3,M2,E2,P5);(A6,I3,M2,E2,P6);(A6,I3,M2,E2,P7);(A6,I3,M2,E2,P8);(A6,I3,M2,E2,P9);(A6,I3,M2,E2,P10);(A6,I3,M2,E2,P11);(A6,I3,M2,E2,P12);(A6,I3,M2,E2,P13);(A6,I3,M2,E2,P14);(A6,I3,M2,E2,P15);(A6,I3,M2,E2,P16);(A6,I3,M2,E2,P17);(A6,I3,M2,E2,P18);(A6,I3,M2,E2,P19);(A6,I3,M2,E2,P20);(A6,I3,M2,E3,P1);(A6,I3,M2,E3,P2);(A6,I3,M2,E3,P3);(A6,I3,M2,E3,P4);(A6,I3,M2,E3,P5);(A6,I3,M2,E3,P6);(A6,I3,M2,E3,P7);(A6,I3,M2,E3,P8);(A6,I3,M2,E3,P9);(A6,I3,M2,E3,P10);(A6,I3,M2,E3,P11);(A6,I3,M2,E3,P12);(A6,I3,M2,E3,P13);(A6,I3,M2,E3,P14);(A6,I3,M2,E3,P15);(A6,I3,M2,E3,P16);(A6,I3,M2,E3,P17);(A6,I3,M2,E3,P18);(A6,I3,M2,E3,P19);(A6,I3,M2,E3,P20);(A6,I3,M2,E4,P1);(A6,I3,M2,E4,P2);(A6,I3,M2,E4,P3);(A6,I3,M2,E4,P4);(A6,I3,M2,E4,P5);(A6,I3,M2,E4,P6);(A6,I3,M2,E4,P7);(A6,I3,M2,E4,P8);(A6,I3,M2,E4,P9);(A6,I3,M2,E4,P10);(A6,I3,M2,E4,P11);(A6,I3,M2,E4,P12);(A6,I3,M2,E4,P13);(A6,I3,M2,E4,P14);(A6,I3,M2,E4,P15);(A6,I3,M2,E4,P16);(A6,I3,M2,E4,P17);(A6,I3,M2,E4,P18);(A6,I3,M2,E4,P19);(A6,I3,M2,E4,P20);(A6,I3,M2,E5,P1);(A6,I3,M2,E5,P2);(A6,I3,M2,E5,P3);(A6,I3,M2,E5,P4);(A6,I3,M2,E5,P5);(A6,I3,M2,E5,P6);(A6,I3,M2,E5,P7);(A6,I3,M2,E5,P8);(A6,I3,M2,E5,P9);(A6,I3,M2,E5,P10);(A6,I3,M2,E5,P11);(A6,I3,M2,E5,P12);(A6,I3,M2,E5,P13);(A6,I3,M2,E5,P14);(A6,I3,M2,E5,P15);(A6,I3,M2,E5,P16);(A6,I3,M2,E5,P17);(A6,I3,M2,E5,P18);(A6,I3,M2,E5,P19);(A6,I3,M2,E5,P20);(A6,I3,M2,E6,P1);(A6,I3,M2,E6,P2);(A6,I3,M2,E6,P3);(A6,I3,M2,E6,P4);(A6,I3,M2,E6,P5);(A6,I3,M2,E6,P6);(A6,I3,M2,E6,P7);(A6,I3,M2,E6,P8);(A6,I3,M2,E6,P9);(A6,I3,M2,E6,P10);(A6,I3,M2,E6,P11);(A6,I3,M2,E6,P12);(A6,I3,M2,E6,P13);(A6,I3,M2,E6,P14);(A6,I3,M2,E6,P15);(A6,I3,M2,E6,P16);(A6,I3,M2,E6,P17);(A6,I3,M2,E6,P18);(A6,I3,M2,E6,P19);(A6,I3,M2,E6,P20);(A6,I3,M2,E7,P1);(A6,I3,M2,E7,P2);(A6,I3,M2,E7,P3);(A6,I3,M2,E7,P4);(A6,I3,M2,E7,P5);(A6,I3,M2,E7,P6);(A6,I3,M2,E7,P7);(A6,I3,M2,E7,P8);(A6,I3,M2,E7,P9);(A6,I3,M2,E7,P10);(A6,I3,M2,E7,P11);(A6,I3,M2,E7,P12);(A6,I3,M2,E7,P13);(A6,I3,M2,E7,P14);(A6,I3,M2,E7,P15);(A6,I3,M2,E7,P16);(A6,I3,M2,E7,P17);(A6,I3,M2,E7,P18);(A6,I3,M2,E7,P19);(A6,I3,M2,E7,P20);(A6,I3,M2,E8,P1);(A6,I3,M2,E8,P2);(A6,I3,M2,E8,P3);(A6,I3,M2,E8,P4);(A6,I3,M2,E8,P5);(A6,I3,M2,E8,P6);(A6,I3,M2,E8,P7);(A6,I3,M2,E8,P8);(A6,I3,M2,E8,P9);(A6,I3,M2,E8,P10);(A6,I3,M2,E8,P11);(A6,I3,M2,E8,P12);(A6,I3,M2,E8,P13);(A6,I3,M2,E8,P14);(A6,I3,M2,E8,P15);(A6,I3,M2,E8,P16);(A6,I3,M2,E8,P17);(A6,I3,M2,E8,P18);(A6,I3,M2,E8,P19);(A6,I3,M2,E8,P20);(A6,I3,M2,E9,P1);(A6,I3,M2,E9,P2);(A6,I3,M2,E9,P3);(A6,I3,M2,E9,P4);(A6,I3,M2,E9,P5);(A6,I3,M2,E9,P6);(A6,I3,M2,E9,P7);(A6,I3,M2,E9,P8);(A6,I3,M2,E9,P9);(A6,I3,M2,E9,P10);(A6,I3,M2,E9,P11);(A6,I3,M2,E9,P12);(A6,I3,M2,E9,P13);(A6,I3,M2,E9,P14);(A6,I3,M2,E9,P15);(A6,I3,M2,E9,P16);(A6,I3,M2,E9,P17);(A6,I3,M2,E9,P18);(A6,I3,M2,E9,P19);(A6,I3,M2,E9,P20);(A6,I3,M3,E1,P1);(A6,I3,M3,E1,P2);(A6,I3,M3,E1,P3);(A6,I3,M3,E1,P4);(A6,I3,M3,E1,P5);(A6,I3,M3,E1,P6);(A6,I3,M3,E1,P7);(A6,I3,M3,E1,P8);(A6,I3,M3,E1,P9);(A6,I3,M3,E1,P10);(A6,I3,M3,E1,P11);(A6,I3,M3,E1,P12);(A6,I3,M3,E1,P13);(A6,I3,M3,E1,P14);(A6,I3,M3,E1,P15);(A6,I3,M3,E1,P16);(A6,I3,M3,E1,P17);(A6,I3,M3,E1,P18);(A6,I3,M3,E1,P19);(A6,I3,M3,E1,P20);(A6,I3,M3,E2,P1);(A6,I3,M3,E2,P2);(A6,I3,M3,E2,P3);(A6,I3,M3,E2,P4);(A6,I3,M3,E2,P5);(A6,I3,M3,E2,P6);(A6,I3,M3,E2,P7);(A6,I3,M3,E2,P8);(A6,I3,M3,E2,P9);(A6,I3,M3,E2,P10);(A6,I3,M3,E2,P11);(A6,I3,M3,E2,P12);(A6,I3,M3,E2,P13);(A6,I3,M3,E2,P14);(A6,I3,M3,E2,P15);(A6,I3,M3,E2,P16);(A6,I3,M3,E2,P17);(A6,I3,M3,E2,P18);(A6,I3,M3,E2,P19);(A6,I3,M3,E2,P20);(A6,I3,M3,E3,P1);(A6,I3,M3,E3,P2);(A6,I3,M3,E3,P3);(A6,I3,M3,E3,P4);(A6,I3,M3,E3,P5);(A6,I3,M3,E3,P6);(A6,I3,M3,E3,P7);(A6,I3,M3,E3,P8);(A6,I3,M3,E3,P9);(A6,I3,M3,E3,P10);(A6,I3,M3,E3,P11);(A6,I3,M3,E3,P12);(A6,I3,M3,E3,P13);(A6,I3,M3,E3,P14);(A6,I3,M3,E3,P15);(A6,I3,M3,E3,P16);(A6,I3,M3,E3,P17);(A6,I3,M3,E3,P18);(A6,I3,M3,E3,P19);(A6,I3,M3,E3,P20);(A6,I3,M3,E4,P1);(A6,I3,M3,E4,P2);(A6,I3,M3,E4,P3);(A6,I3,M3,E4,P4);(A6,I3,M3,E4,P5);(A6,I3,M3,E4,P6);(A6,I3,M3,E4,P7);(A6,I3,M3,E4,P8);(A6,I3,M3,E4,P9);(A6,I3,M3,E4,P10);(A6,I3,M3,E4,P11);(A6,I3,M3,E4,P12);(A6,I3,M3,E4,P13);(A6,I3,M3,E4,P14);(A6,I3,M3,E4,P15);(A6,I3,M3,E4,P16);(A6,I3,M3,E4,P17);(A6,I3,M3,E4,P18);(A6,I3,M3,E4,P19);(A6,I3,M3,E4,P20);(A6,I3,M3,E5,P1);(A6,I3,M3,E5,P2);(A6,I3,M3,E5,P3);(A6,I3,M3,E5,P4);(A6,I3,M3,E5,P5);(A6,I3,M3,E5,P6);(A6,I3,M3,E5,P7);(A6,I3,M3,E5,P8);(A6,I3,M3,E5,P9);(A6,I3,M3,E5,P10);(A6,I3,M3,E5,P11);(A6,I3,M3,E5,P12);(A6,I3,M3,E5,P13);(A6,I3,M3,E5,P14);(A6,I3,M3,E5,P15);(A6,I3,M3,E5,P16);(A6,I3,M3,E5,P17);(A6,I3,M3,E5,P18);(A6,I3,M3,E5,P19);(A6,I3,M3,E5,P20);(A6,I3,M3,E6,P1);(A6,I3,M3,E6,P2);(A6,I3,M3,E6,P3);(A6,I3,M3,E6,P4);(A6,I3,M3,E6,P5);(A6,I3,M3,E6,P6);(A6,I3,M3,E6,P7);(A6,I3,M3,E6,P8);(A6,I3,M3,E6,P9);(A6,I3,M3,E6,P10);(A6,I3,M3,E6,P11);(A6,I3,M3,E6,P12);(A6,I3,M3,E6,P13);(A6,I3,M3,E6,P14);(A6,I3,M3,E6,P15);(A6,I3,M3,E6,P16);(A6,I3,M3,E6,P17);(A6,I3,M3,E6,P18);(A6,I3,M3,E6,P19);(A6,I3,M3,E6,P20);(A6,I3,M3,E7,P1);(A6,I3,M3,E7,P2);(A6,I3,M3,E7,P3);(A6,I3,M3,E7,P4);(A6,I3,M3,E7,P5);(A6,I3,M3,E7,P6);(A6,I3,M3,E7,P7);(A6,I3,M3,E7,P8);(A6,I3,M3,E7,P9);(A6,I3,M3,E7,P10);(A6,I3,M3,E7,P11);(A6,I3,M3,E7,P12);(A6,I3,M3,E7,P13);(A6,I3,M3,E7,P14);(A6,I3,M3,E7,P15);(A6,I3,M3,E7,P16);(A6,I3,M3,E7,P17);(A6,I3,M3,E7,P18);(A6,I3,M3,E7,P19);(A6,I3,M3,E7,P20);(A6,I3,M3,E8,P1);(A6,I3,M3,E8,P2);(A6,I3,M3,E8,P3);(A6,I3,M3,E8,P4);(A6,I3,M3,E8,P5);(A6,I3,M3,E8,P6);(A6,I3,M3,E8,P7);(A6,I3,M3,E8,P8);(A6,I3,M3,E8,P9);(A6,I3,M3,E8,P10);(A6,I3,M3,E8,P11);(A6,I3,M3,E8,P12);(A6,I3,M3,E8,P13);(A6,I3,M3,E8,P14);(A6,I3,M3,E8,P15);(A6,I3,M3,E8,P16);(A6,I3,M3,E8,P17);(A6,I3,M3,E8,P18);(A6,I3,M3,E8,P19);(A6,I3,M3,E8,P20);(A6,I3,M3,E9,P1);(A6,I3,M3,E9,P2);(A6,I3,M3,E9,P3);(A6,I3,M3,E9,P4);(A6,I3,M3,E9,P5);(A6,I3,M3,E9,P6);(A6,I3,M3,E9,P7);(A6,I3,M3,E9,P8);(A6,I3,M3,E9,P9);(A6,I3,M3,E9,P10);(A6,I3,M3,E9,P11);(A6,I3,M3,E9,P12);(A6,I3,M3,E9,P13);(A6,I3,M3,E9,P14);(A6,I3,M3,E9,P15);(A6,I3,M3,E9,P16);(A6,I3,M3,E9,P17);(A6,I3,M3,E9,P18);(A6,I3,M3,E9,P19);(A6,I3,M3,E9,P20);(A6,I3,M4,E1,P1);(A6,I3,M4,E1,P2);(A6,I3,M4,E1,P3);(A6,I3,M4,E1,P4);(A6,I3,M4,E1,P5);(A6,I3,M4,E1,P6);(A6,I3,M4,E1,P7);(A6,I3,M4,E1,P8);(A6,I3,M4,E1,P9);(A6,I3,M4,E1,P10);(A6,I3,M4,E1,P11);(A6,I3,M4,E1,P12);(A6,I3,M4,E1,P13);(A6,I3,M4,E1,P14);(A6,I3,M4,E1,P15);(A6,I3,M4,E1,P16);(A6,I3,M4,E1,P17);(A6,I3,M4,E1,P18);(A6,I3,M4,E1,P19);(A6,I3,M4,E1,P20);(A6,I3,M4,E2,P1);(A6,I3,M4,E2,P2);(A6,I3,M4,E2,P3);(A6,I3,M4,E2,P4);(A6,I3,M4,E2,P5);(A6,I3,M4,E2,P6);(A6,I3,M4,E2,P7);(A6,I3,M4,E2,P8);(A6,I3,M4,E2,P9);(A6,I3,M4,E2,P10);(A6,I3,M4,E2,P11);(A6,I3,M4,E2,P12);(A6,I3,M4,E2,P13);(A6,I3,M4,E2,P14);(A6,I3,M4,E2,P15);(A6,I3,M4,E2,P16);(A6,I3,M4,E2,P17);(A6,I3,M4,E2,P18);(A6,I3,M4,E2,P19);(A6,I3,M4,E2,P20);(A6,I3,M4,E3,P1);(A6,I3,M4,E3,P2);(A6,I3,M4,E3,P3);(A6,I3,M4,E3,P4);(A6,I3,M4,E3,P5);(A6,I3,M4,E3,P6);(A6,I3,M4,E3,P7);(A6,I3,M4,E3,P8);(A6,I3,M4,E3,P9);(A6,I3,M4,E3,P10);(A6,I3,M4,E3,P11);(A6,I3,M4,E3,P12);(A6,I3,M4,E3,P13);(A6,I3,M4,E3,P14);(A6,I3,M4,E3,P15);(A6,I3,M4,E3,P16);(A6,I3,M4,E3,P17);(A6,I3,M4,E3,P18);(A6,I3,M4,E3,P19);(A6,I3,M4,E3,P20);(A6,I3,M4,E4,P1);(A6,I3,M4,E4,P2);(A6,I3,M4,E4,P3);(A6,I3,M4,E4,P4);(A6,I3,M4,E4,P5);(A6,I3,M4,E4,P6);(A6,I3,M4,E4,P7);(A6,I3,M4,E4,P8);(A6,I3,M4,E4,P9);(A6,I3,M4,E4,P10);(A6,I3,M4,E4,P11);(A6,I3,M4,E4,P12);(A6,I3,M4,E4,P13);(A6,I3,M4,E4,P14);(A6,I3,M4,E4,P15);(A6,I3,M4,E4,P16);(A6,I3,M4,E4,P17);(A6,I3,M4,E4,P18);(A6,I3,M4,E4,P19);(A6,I3,M4,E4,P20);(A6,I3,M4,E5,P1);(A6,I3,M4,E5,P2);(A6,I3,M4,E5,P3);(A6,I3,M4,E5,P4);(A6,I3,M4,E5,P5);(A6,I3,M4,E5,P6);(A6,I3,M4,E5,P7);(A6,I3,M4,E5,P8);(A6,I3,M4,E5,P9);(A6,I3,M4,E5,P10);(A6,I3,M4,E5,P11);(A6,I3,M4,E5,P12);(A6,I3,M4,E5,P13);(A6,I3,M4,E5,P14);(A6,I3,M4,E5,P15);(A6,I3,M4,E5,P16);(A6,I3,M4,E5,P17);(A6,I3,M4,E5,P18);(A6,I3,M4,E5,P19);(A6,I3,M4,E5,P20);(A6,I3,M4,E6,P1);(A6,I3,M4,E6,P2);(A6,I3,M4,E6,P3);(A6,I3,M4,E6,P4);(A6,I3,M4,E6,P5);(A6,I3,M4,E6,P6);(A6,I3,M4,E6,P7);(A6,I3,M4,E6,P8);(A6,I3,M4,E6,P9);(A6,I3,M4,E6,P10);(A6,I3,M4,E6,P11);(A6,I3,M4,E6,P12);(A6,I3,M4,E6,P13);(A6,I3,M4,E6,P14);(A6,I3,M4,E6,P15);(A6,I3,M4,E6,P16);(A6,I3,M4,E6,P17);(A6,I3,M4,E6,P18);(A6,I3,M4,E6,P19);(A6,I3,M4,E6,P20);(A6,I3,M4,E7,P1);(A6,I3,M4,E7,P2);(A6,I3,M4,E7,P3);(A6,I3,M4,E7,P4);(A6,I3,M4,E7,P5);(A6,I3,M4,E7,P6);(A6,I3,M4,E7,P7);(A6,I3,M4,E7,P8);(A6,I3,M4,E7,P9);(A6,I3,M4,E7,P10);(A6,I3,M4,E7,P11);(A6,I3,M4,E7,P12);(A6,I3,M4,E7,P13);(A6,I3,M4,E7,P14);(A6,I3,M4,E7,P15);(A6,I3,M4,E7,P16);(A6,I3,M4,E7,P17);(A6,I3,M4,E7,P18);(A6,I3,M4,E7,P19);(A6,I3,M4,E7,P20);(A6,I3,M4,E8,P1);(A6,I3,M4,E8,P2);(A6,I3,M4,E8,P3);(A6,I3,M4,E8,P4);(A6,I3,M4,E8,P5);(A6,I3,M4,E8,P6);(A6,I3,M4,E8,P7);(A6,I3,M4,E8,P8);(A6,I3,M4,E8,P9);(A6,I3,M4,E8,P10);(A6,I3,M4,E8,P11);(A6,I3,M4,E8,P12);(A6,I3,M4,E8,P13);(A6,I3,M4,E8,P14);(A6,I3,M4,E8,P15);(A6,I3,M4,E8,P16);(A6,I3,M4,E8,P17);(A6,I3,M4,E8,P18);(A6,I3,M4,E8,P19);(A6,I3,M4,E8,P20);(A6,I3,M4,E9,P1);(A6,I3,M4,E9,P2);(A6,I3,M4,E9,P3);(A6,I3,M4,E9,P4);(A6,I3,M4,E9,P5);(A6,I3,M4,E9,P6);(A6,I3,M4,E9,P7);(A6,I3,M4,E9,P8);(A6,I3,M4,E9,P9);(A6,I3,M4,E9,P10);(A6,I3,M4,E9,P11);(A6,I3,M4,E9,P12);(A6,I3,M4,E9,P13);(A6,I3,M4,E9,P14);(A6,I3,M4,E9,P15);(A6,I3,M4,E9,P16);(A6,I3,M4,E9,P17);(A6,I3,M4,E9,P18);(A6,I3,M4,E9,P19);(A6,I3,M4,E9,P20);(A6,I3,M5,E1,P1);(A6,I3,M5,E1,P2);(A6,I3,M5,E1,P3);(A6,I3,M5,E1,P4);(A6,I3,M5,E1,P5);(A6,I3,M5,E1,P6);(A6,I3,M5,E1,P7);(A6,I3,M5,E1,P8);(A6,I3,M5,E1,P9);(A6,I3,M5,E1,P10);(A6,I3,M5,E1,P11);(A6,I3,M5,E1,P12);(A6,I3,M5,E1,P13);(A6,I3,M5,E1,P14);(A6,I3,M5,E1,P15);(A6,I3,M5,E1,P16);(A6,I3,M5,E1,P17);(A6,I3,M5,E1,P18);(A6,I3,M5,E1,P19);(A6,I3,M5,E1,P20);(A6,I3,M5,E2,P1);(A6,I3,M5,E2,P2);(A6,I3,M5,E2,P3);(A6,I3,M5,E2,P4);(A6,I3,M5,E2,P5);(A6,I3,M5,E2,P6);(A6,I3,M5,E2,P7);(A6,I3,M5,E2,P8);(A6,I3,M5,E2,P9);(A6,I3,M5,E2,P10);(A6,I3,M5,E2,P11);(A6,I3,M5,E2,P12);(A6,I3,M5,E2,P13);(A6,I3,M5,E2,P14);(A6,I3,M5,E2,P15);(A6,I3,M5,E2,P16);(A6,I3,M5,E2,P17);(A6,I3,M5,E2,P18);(A6,I3,M5,E2,P19);(A6,I3,M5,E2,P20);(A6,I3,M5,E3,P1);(A6,I3,M5,E3,P2);(A6,I3,M5,E3,P3);(A6,I3,M5,E3,P4);(A6,I3,M5,E3,P5);(A6,I3,M5,E3,P6);(A6,I3,M5,E3,P7);(A6,I3,M5,E3,P8);(A6,I3,M5,E3,P9);(A6,I3,M5,E3,P10);(A6,I3,M5,E3,P11);(A6,I3,M5,E3,P12);(A6,I3,M5,E3,P13);(A6,I3,M5,E3,P14);(A6,I3,M5,E3,P15);(A6,I3,M5,E3,P16);(A6,I3,M5,E3,P17);(A6,I3,M5,E3,P18);(A6,I3,M5,E3,P19);(A6,I3,M5,E3,P20);(A6,I3,M5,E4,P1);(A6,I3,M5,E4,P2);(A6,I3,M5,E4,P3);(A6,I3,M5,E4,P4);(A6,I3,M5,E4,P5);(A6,I3,M5,E4,P6);(A6,I3,M5,E4,P7);(A6,I3,M5,E4,P8);(A6,I3,M5,E4,P9);(A6,I3,M5,E4,P10);(A6,I3,M5,E4,P11);(A6,I3,M5,E4,P12);(A6,I3,M5,E4,P13);(A6,I3,M5,E4,P14);(A6,I3,M5,E4,P15);(A6,I3,M5,E4,P16);(A6,I3,M5,E4,P17);(A6,I3,M5,E4,P18);(A6,I3,M5,E4,P19);(A6,I3,M5,E4,P20);(A6,I3,M5,E5,P1);(A6,I3,M5,E5,P2);(A6,I3,M5,E5,P3);(A6,I3,M5,E5,P4);(A6,I3,M5,E5,P5);(A6,I3,M5,E5,P6);(A6,I3,M5,E5,P7);(A6,I3,M5,E5,P8);(A6,I3,M5,E5,P9);(A6,I3,M5,E5,P10);(A6,I3,M5,E5,P11);(A6,I3,M5,E5,P12);(A6,I3,M5,E5,P13);(A6,I3,M5,E5,P14);(A6,I3,M5,E5,P15);(A6,I3,M5,E5,P16);(A6,I3,M5,E5,P17);(A6,I3,M5,E5,P18);(A6,I3,M5,E5,P19);(A6,I3,M5,E5,P20);(A6,I3,M5,E6,P1);(A6,I3,M5,E6,P2);(A6,I3,M5,E6,P3);(A6,I3,M5,E6,P4);(A6,I3,M5,E6,P5);(A6,I3,M5,E6,P6);(A6,I3,M5,E6,P7);(A6,I3,M5,E6,P8);(A6,I3,M5,E6,P9);(A6,I3,M5,E6,P10);(A6,I3,M5,E6,P11);(A6,I3,M5,E6,P12);(A6,I3,M5,E6,P13);(A6,I3,M5,E6,P14);(A6,I3,M5,E6,P15);(A6,I3,M5,E6,P16);(A6,I3,M5,E6,P17);(A6,I3,M5,E6,P18);(A6,I3,M5,E6,P19);(A6,I3,M5,E6,P20);(A6,I3,M5,E7,P1);(A6,I3,M5,E7,P2);(A6,I3,M5,E7,P3);(A6,I3,M5,E7,P4);(A6,I3,M5,E7,P5);(A6,I3,M5,E7,P6);(A6,I3,M5,E7,P7);(A6,I3,M5,E7,P8);(A6,I3,M5,E7,P9);(A6,I3,M5,E7,P10);(A6,I3,M5,E7,P11);(A6,I3,M5,E7,P12);(A6,I3,M5,E7,P13);(A6,I3,M5,E7,P14);(A6,I3,M5,E7,P15);(A6,I3,M5,E7,P16);(A6,I3,M5,E7,P17);(A6,I3,M5,E7,P18);(A6,I3,M5,E7,P19);(A6,I3,M5,E7,P20);(A6,I3,M5,E8,P1);(A6,I3,M5,E8,P2);(A6,I3,M5,E8,P3);(A6,I3,M5,E8,P4);(A6,I3,M5,E8,P5);(A6,I3,M5,E8,P6);(A6,I3,M5,E8,P7);(A6,I3,M5,E8,P8);(A6,I3,M5,E8,P9);(A6,I3,M5,E8,P10);(A6,I3,M5,E8,P11);(A6,I3,M5,E8,P12);(A6,I3,M5,E8,P13);(A6,I3,M5,E8,P14);(A6,I3,M5,E8,P15);(A6,I3,M5,E8,P16);(A6,I3,M5,E8,P17);(A6,I3,M5,E8,P18);(A6,I3,M5,E8,P19);(A6,I3,M5,E8,P20);(A6,I3,M5,E9,P1);(A6,I3,M5,E9,P2);(A6,I3,M5,E9,P3);(A6,I3,M5,E9,P4);(A6,I3,M5,E9,P5);(A6,I3,M5,E9,P6);(A6,I3,M5,E9,P7);(A6,I3,M5,E9,P8);(A6,I3,M5,E9,P9);(A6,I3,M5,E9,P10);(A6,I3,M5,E9,P11);(A6,I3,M5,E9,P12);(A6,I3,M5,E9,P13);(A6,I3,M5,E9,P14);(A6,I3,M5,E9,P15);(A6,I3,M5,E9,P16);(A6,I3,M5,E9,P17);(A6,I3,M5,E9,P18);(A6,I3,M5,E9,P19);(A6,I3,M5,E9,P20);(A6,I3,M6,E1,P1);(A6,I3,M6,E1,P2);(A6,I3,M6,E1,P3);(A6,I3,M6,E1,P4);(A6,I3,M6,E1,P5);(A6,I3,M6,E1,P6);(A6,I3,M6,E1,P7);(A6,I3,M6,E1,P8);(A6,I3,M6,E1,P9);(A6,I3,M6,E1,P10);(A6,I3,M6,E1,P11);(A6,I3,M6,E1,P12);(A6,I3,M6,E1,P13);(A6,I3,M6,E1,P14);(A6,I3,M6,E1,P15);(A6,I3,M6,E1,P16);(A6,I3,M6,E1,P17);(A6,I3,M6,E1,P18);(A6,I3,M6,E1,P19);(A6,I3,M6,E1,P20);(A6,I3,M6,E2,P1);(A6,I3,M6,E2,P2);(A6,I3,M6,E2,P3);(A6,I3,M6,E2,P4);(A6,I3,M6,E2,P5);(A6,I3,M6,E2,P6);(A6,I3,M6,E2,P7);(A6,I3,M6,E2,P8);(A6,I3,M6,E2,P9);(A6,I3,M6,E2,P10);(A6,I3,M6,E2,P11);(A6,I3,M6,E2,P12);(A6,I3,M6,E2,P13);(A6,I3,M6,E2,P14);(A6,I3,M6,E2,P15);(A6,I3,M6,E2,P16);(A6,I3,M6,E2,P17);(A6,I3,M6,E2,P18);(A6,I3,M6,E2,P19);(A6,I3,M6,E2,P20);(A6,I3,M6,E3,P1);(A6,I3,M6,E3,P2);(A6,I3,M6,E3,P3);(A6,I3,M6,E3,P4);(A6,I3,M6,E3,P5);(A6,I3,M6,E3,P6);(A6,I3,M6,E3,P7);(A6,I3,M6,E3,P8);(A6,I3,M6,E3,P9);(A6,I3,M6,E3,P10);(A6,I3,M6,E3,P11);(A6,I3,M6,E3,P12);(A6,I3,M6,E3,P13);(A6,I3,M6,E3,P14);(A6,I3,M6,E3,P15);(A6,I3,M6,E3,P16);(A6,I3,M6,E3,P17);(A6,I3,M6,E3,P18);(A6,I3,M6,E3,P19);(A6,I3,M6,E3,P20);(A6,I3,M6,E4,P1);(A6,I3,M6,E4,P2);(A6,I3,M6,E4,P3);(A6,I3,M6,E4,P4);(A6,I3,M6,E4,P5);(A6,I3,M6,E4,P6);(A6,I3,M6,E4,P7);(A6,I3,M6,E4,P8);(A6,I3,M6,E4,P9);(A6,I3,M6,E4,P10);(A6,I3,M6,E4,P11);(A6,I3,M6,E4,P12);(A6,I3,M6,E4,P13);(A6,I3,M6,E4,P14);(A6,I3,M6,E4,P15);(A6,I3,M6,E4,P16);(A6,I3,M6,E4,P17);(A6,I3,M6,E4,P18);(A6,I3,M6,E4,P19);(A6,I3,M6,E4,P20);(A6,I3,M6,E5,P1);(A6,I3,M6,E5,P2);(A6,I3,M6,E5,P3);(A6,I3,M6,E5,P4);(A6,I3,M6,E5,P5);(A6,I3,M6,E5,P6);(A6,I3,M6,E5,P7);(A6,I3,M6,E5,P8);(A6,I3,M6,E5,P9);(A6,I3,M6,E5,P10);(A6,I3,M6,E5,P11);(A6,I3,M6,E5,P12);(A6,I3,M6,E5,P13);(A6,I3,M6,E5,P14);(A6,I3,M6,E5,P15);(A6,I3,M6,E5,P16);(A6,I3,M6,E5,P17);(A6,I3,M6,E5,P18);(A6,I3,M6,E5,P19);(A6,I3,M6,E5,P20);(A6,I3,M6,E6,P1);(A6,I3,M6,E6,P2);(A6,I3,M6,E6,P3);(A6,I3,M6,E6,P4);(A6,I3,M6,E6,P5);(A6,I3,M6,E6,P6);(A6,I3,M6,E6,P7);(A6,I3,M6,E6,P8);(A6,I3,M6,E6,P9);(A6,I3,M6,E6,P10);(A6,I3,M6,E6,P11);(A6,I3,M6,E6,P12);(A6,I3,M6,E6,P13);(A6,I3,M6,E6,P14);(A6,I3,M6,E6,P15);(A6,I3,M6,E6,P16);(A6,I3,M6,E6,P17);(A6,I3,M6,E6,P18);(A6,I3,M6,E6,P19);(A6,I3,M6,E6,P20);(A6,I3,M6,E7,P1);(A6,I3,M6,E7,P2);(A6,I3,M6,E7,P3);(A6,I3,M6,E7,P4);(A6,I3,M6,E7,P5);(A6,I3,M6,E7,P6);(A6,I3,M6,E7,P7);(A6,I3,M6,E7,P8);(A6,I3,M6,E7,P9);(A6,I3,M6,E7,P10);(A6,I3,M6,E7,P11);(A6,I3,M6,E7,P12);(A6,I3,M6,E7,P13);(A6,I3,M6,E7,P14);(A6,I3,M6,E7,P15);(A6,I3,M6,E7,P16);(A6,I3,M6,E7,P17);(A6,I3,M6,E7,P18);(A6,I3,M6,E7,P19);(A6,I3,M6,E7,P20);(A6,I3,M6,E8,P1);(A6,I3,M6,E8,P2);(A6,I3,M6,E8,P3);(A6,I3,M6,E8,P4);(A6,I3,M6,E8,P5);(A6,I3,M6,E8,P6);(A6,I3,M6,E8,P7);(A6,I3,M6,E8,P8);(A6,I3,M6,E8,P9);(A6,I3,M6,E8,P10);(A6,I3,M6,E8,P11);(A6,I3,M6,E8,P12);(A6,I3,M6,E8,P13);(A6,I3,M6,E8,P14);(A6,I3,M6,E8,P15);(A6,I3,M6,E8,P16);(A6,I3,M6,E8,P17);(A6,I3,M6,E8,P18);(A6,I3,M6,E8,P19);(A6,I3,M6,E8,P20);(A6,I3,M6,E9,P1);(A6,I3,M6,E9,P2);(A6,I3,M6,E9,P3);(A6,I3,M6,E9,P4);(A6,I3,M6,E9,P5);(A6,I3,M6,E9,P6);(A6,I3,M6,E9,P7);(A6,I3,M6,E9,P8);(A6,I3,M6,E9,P9);(A6,I3,M6,E9,P10);(A6,I3,M6,E9,P11);(A6,I3,M6,E9,P12);(A6,I3,M6,E9,P13);(A6,I3,M6,E9,P14);(A6,I3,M6,E9,P15);(A6,I3,M6,E9,P16);(A6,I3,M6,E9,P17);(A6,I3,M6,E9,P18);(A6,I3,M6,E9,P19);(A6,I3,M6,E9,P20)

### EXAMPLES

In order that the invention described herein may be more fully understood, the following examples are set forth. The synthetic and biological examples described in this application are offered to illustrate the methods provided herein and are not to be construed in any way as limiting their scope.

### Example 1

### Cell Free Conversion of Starch to Allulose

This example describes the conversion of starch to allulose. Cells (e.g., bacterial or yeast cells) engineered to express at least one heterologous genes encoding at least one enzyme for the conversion of starch to allulose are grown in liquid cultures to high cell density. Examples of heterologous enzymes that may be used in this example include thermostable variants of a α-glucan phosphorylase, a phosphoglucomutase, a phosphoglucoisomerase, an allulose 6-phosphate epimerase, and an allulose 6-phosphate phosphatase. At the end of the growth stage, expression of the heterologous enzyme(s) is induced, and the cell biomass is subsequently harvested. The harvested biomass is then lysed via mechanical, chemical or enzymatic means. The cell lysate is then heated to a temperature that inactivates native enzymatic activities but does not inactivate the heterologous enzyme(s). A starch feedstock, inorganic phosphate and optionally other additional nutrients are added to the heat inactivated lysate to enable conversion of polysaccharide to allulose (FIG. 1).

### Example 2

### Cell Free Conversion of Cellulose to Allulose

This example describes the conversion of cellulose/cellodextrin to allulose. Cells (e.g., bacterial or yeast cells) engineered to express at least one heterologous genes encoding at least one enzyme for the conversion of starch to allulose are grown in liquid cultures to high cell density. Examples of heterologous enzymes that may be used in this example include thermostable variants of a cellodextrin phosphorylase, a phosphoglucomutase, a phosphoglucoisomerase, an allulose 6-phosphate epimerase, and an allulose 6-phosphate phosphatase. At the end of the growth stage, expression of the heterologous enzyme(s) is induced, and the cell biomass is subsequently harvested. The harvested biomass is then lysed via mechanical, chemical or enzymatic means. The cell lysate is then heated to a temperature that inactivates native enzymatic activities but does not inactivate the heterologous enzyme(s). A cellulose/cellodextrin feedstock, inorganic phosphate and optionally other additional nutrients are added to the heat inactivated lysate to enable conversion of cellulose/cellodextrin to allulose (FIG. 2).

### Example 3

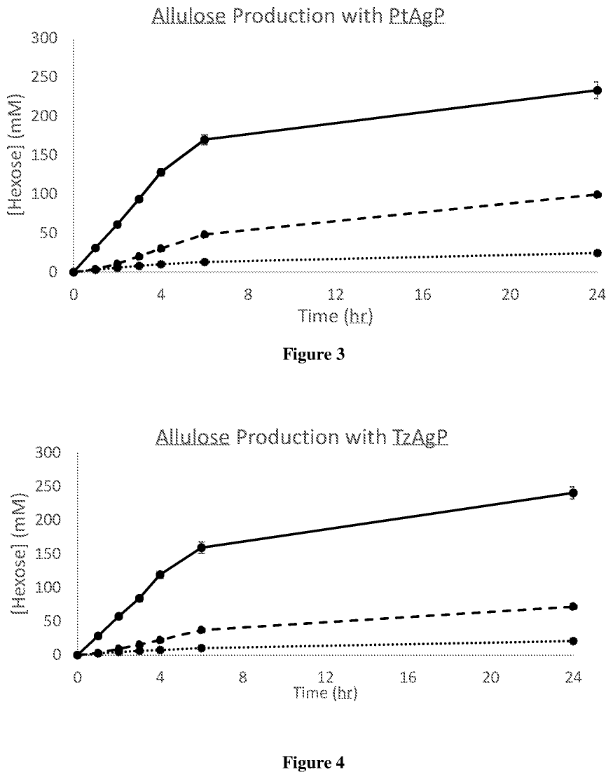

### Cell Free Production of Starch to Allulose Using α-Glucan Phosphorylase Taken from (a) Pseudothermotoga thermarum (PtAgp) (FIG. 3), and (b) Thermococcus zilligii (TzAgp) (FIG. 4)

This example describes the conversion of starch to allulose using specific α-glucan phosphorylases, PtAgp (FIG. 3) and TzAgp (FIG. 4). Lysates were mixed with 100 mM potassium phosphate buffer (pH=7.0), 100 mM NaCl, 2 mM MgCl₂, 2 mM MnCl₂, and 0.5 mM CoCl₂ and then heat-treated at 60° C. for 40 minutes prior to initiating the reaction, to remove deleterious background activities from the lysate. Reactions were then initiated by spiking in 100 g/L maltodextrin and incubated at 60° C. over 24 hours, sampling periodically to measure allulose. Sampling was performed by removing sample of the lysate cocktail, quenching in trichloroacetic acid, neutralizing the supernatant with potassium hydroxide, and storing that solution at −80° C. until analyzed by LC-QQQ mass spec for sugar quantitation.

### Example 4

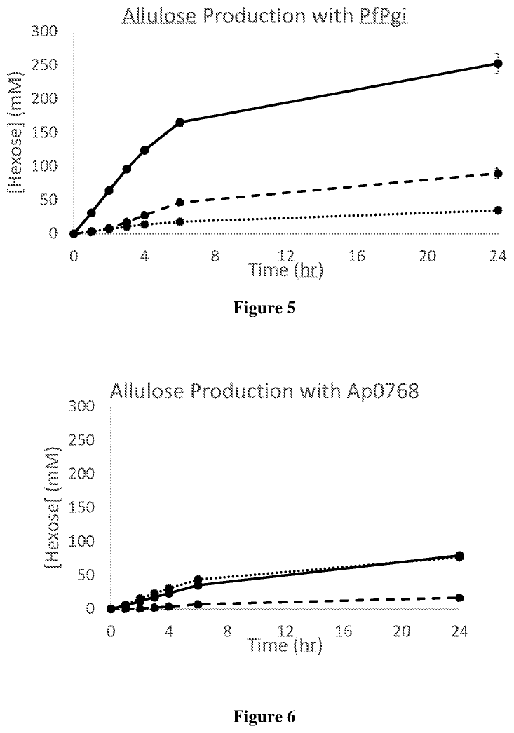

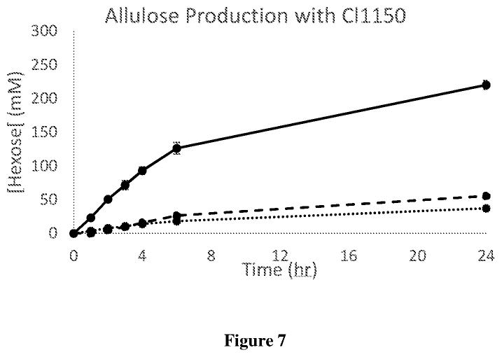

### Cell Free Production of Starch to Allulose Using Phosphoglucoisomerases Taken from from (a) Pyrococcus furiosus (PfPgi) (FIG. 5) (b) Aeropyrum pernix (Ap0768) (FIG. 6) and (c) Caldisphaera lagunensis (Cl1150) (FIG. 7)

This example describes the conversion of starch to allulose using specific phosphoglucoisomerases: PfPgi (FIG. 5), Ap0 768 (FIG. 6), and Cl1150 (FIG. 7). Lysates were mixed with 100 mM potassium phosphate buffer (pH=7.0), 100 mM NaCl, 2 mM MgCl₂, 2 mM MnCl₂, and 0.5 mM CoCl₂ and then heat-treated at 60° C. for 40 minutes prior to initiating the reaction, to remove deleterious background activities from the lysate. Reactions were then initiated by spiking in 100 g/L maltodextrin and incubated at 60° C. over 24 hours, sampling periodically to measure allulose. Sampling was performed by removing sample of the lysate cocktail, quenching in trichloroacetic acid, neutralizing the supernatant with potassium hydroxide, and storing that solution at −80° C. until analyzed by LC-QQQ mass spec for sugar quantitation.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should also be understood that, unless clearly indicated to the contrary, in any methods claimed herein that include more than one step or act, the order of the steps or acts of the method is not necessarily limited to the order in which the steps or acts of the method are recited. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A cell-free method of producing glucose 1-phosphate from a polysaccharide, the method comprising:
converting a polysaccharide to glucose 1-phosphate using an α-glucan phosphorylase selected from the group consisting of AaGlgp (derived from Aquifex aeolicus), TzAgp (derived from Thermococcus zilligii), PtAgp (derived from Pseudothermotoga thermarum), Tm08495 (derived from Thermotoga maritima), TcGlgP (derived from Thermus caldophilus) and PfAgp (derived from Pyrococcus furiosus).

2. The method of claim 1, wherein the α-glucan phosphorylase is selected from the group consisting of TzAgp (derived from Thermococcus zilligii), and PfAgp (derived from Pyrococcus furiosus).

3. The method of claim 1, wherein the α-glucan phosphorylase is selected from the group consisting of AaGlgp (derived from Aquifex aeolicus), TzAgp (derived from Thermococcus zilligii), and PtAgp (derived from Pseudothermotoga thermarum).

4. The method of claim 1, wherein converting the polysaccharide to glucose 1-phosphate further comprises using a debranching enzyme.

5. The method of claim 4, wherein the debranching enzyme is a pullulanase, isoamylase, or a combination thereof.

6. The method of claim 5, wherein the pullulanase is selected from the group consisting of Fp1793 (derived from Fervidobacterium pennavorans), BfPull (derived from Bacillus flavocaldarius), and TRQ5Pul (derived from Thermotoga sp. RQ5).

7. The method of claim 5, wherein the isoamylase is selected from the group consisting of StTreX (derived from Sulfolobus tokodaii), MhTreX (derived from Metallosphaera hakonensis), and StGlgX (derived from Sphaerobacter thermophilus).

8. The method of claim 5, wherein the isoamylase is StTreX (derived from Sulfolobus tokodaii).

9. The method of claim 1 further comprising:
converting glucose 1-phosphate to glucose 6-phosphate using a phosphoglucomutase.

10. The method of claim 9, wherein the phosphoglucomutase is selected from the group consisting of Tk1621 (derived from Thermococcus kodakaraensis), Pk02350 (derived from Pyrococcus kukulkanii), Af0458 (derived from Archaeoglobus fulgidus), CtPgm2 (derived from Clostridium thermocellum), TtPgm2 (derived from Thermus thermophilus), and TiManB (derived from Thermus islandicus).

11. The method of claim 9, wherein the phosphoglucomutase is selected from the group consisting of Tk1621 (derived from Thermococcus kodakaraensis), Pk02350 (derived from Pyrococcus kukulkanii), and CtPgm2 (derived from Clostridium thermocellum).

12. The method of claim 9 further comprising:
converting glucose 6-phosphate to fructose 6-phosphate using a phosphoglucoisomerase.

13. The method of claim 12, wherein the phosphoglucoisomerase is selected from the group consisting of CtPgi (derived from Clostridium thermocellum), TtPgi (derived from Thermus thermophilus), MjPgi (derived from Methanococcus jannaschii), PfPgi (derived from Pyrococcus furiosus), Ap0768 (derived from Aeropyrum pernix), and Cl1150 (derived from Caldisphaera lagunensis).

14. The method of claim 12, wherein the phosphoglucoisomerase is selected from the group consisting of PfPgi (derived from Pyrococcus furiosus), Ap0768 (derived from Aeropyrum pernix), and Cl1150 (derived from Caldisphaera lagunensis).

15. The method of claim 12, wherein the phosphoglucoisomerase is selected from the group consisting of CtPgi (derived from Clostridium thermocellum), TtPgi (derived from Thermus thermophilus), and PfPgi (derived from Pyrococcus furiosus).

16. The method of claim 12 further comprising:
converting fructose 6-phosphate to allulose 6-phosphate using an allulose 6-phosphate epimerase.

17. The method of claim 16, wherein the allulose 6-phosphate epimerase is selected from the group consisting of BtAlsE (derived from Brevibacillus thermoruber), Thebr1340 (derived from Thermoanaerobacter brockii), CasuRpe2 (derived from Caldanaerobacter subterraneus), Defds2125 (derived from Deferribacter desulfuricans), Hg1285 (derived from Hydrogenivirga sp. 128-5-R1-1), Tthe1731 (derived from Thermoanerobacterium thermosaccharolyticum), Theru00510 (derived from Thermocrinis ruber), That0313 (derived from Thermosipho atlanticus), and ThtaRpe (derived from Thermosulfidibacter takaii).

18. The method of claim 16, wherein the allulose 6-phosphate epimerase is selected from the group consisting of BtAlsE (derived from Brevibacillus thermoruber), Hg1285 (derived from Hydrogenivirga sp. 128-5-R1-1), and Tthe1731 (derived from Thermoanerobacterium thermosaccharolyticum).

19. The method of claim 16 further comprising:
converting allulose 6-phosphate to allulose using an allulose 6-phosphate phosphatase.

20. The method of claim 19, wherein the allulose 6-phosphate phosphatase is selected from the group consisting of AaGph (derived from Aquifex aeolicus), Acel0099 (derived from Acidothermus cellulolyticus), CsPpaX (derived from Caldanaerobacter subterraneus), Cthe0261 (derived from Clostridium thermocellum), Desku1269 (derived from Desulfotomaculum kuznetsovii), Dgeo0096 (derived from Deinococcus geothermalis DSM 11300), Mth1760 (derived from Methanothermobacter thermautotrophicus), Pho459 (derived from Pyrococcus horikoshii Ot3), Pmob0141 (derived from Petrotoga mobilis), Tagg0346 (derived from Thermosphaera aggregans), Tfu0224 (derived from Thermobifida fusca), Theet2005 (derived from Thermoanaerobacter ethanolicus), Thein1775 (derived from Thermodesulfatator indicus), TheisHAD (derived from Thermus islandicus), Thewi1735 (derived from Thermoanaerobacter wiegelii), TtC1471 (derived from Thermus thermophilus), Vdis0326 (derived from Crenarchaeota archaeon 13_1_40CM_3_53_5), Bf9343 (derived from Bacteroides fragilis), Bvu4110 (derived from Bacteroides vulgatus), and Ctn1320 (derived from Thermotoga neapolitana).

21. The method of claim 19, wherein the allulose 6-phosphate phosphatase is selected from the group consisting of Cthe0261 (derived from Clostridium thermocellum), Bf9343 (derived from Bacteroides fragilis) and Bvu4110 (derived from Bacteroides vulgatus).

22. The method of claim 1, wherein the polysaccharide is maltodextrin, starch, glycogen, cellulose or cellodextrin.

23. A cell-free method of producing fructose 6-phosphate from glucose 6-phosphate, the method comprising:
converting glucose 6-phosphate to fructose 6-phosphate using a phosphoglucoisomerase selected from the group consisting of CtPgi (derived from Clostridium thermocellum), TtPgi (derived from Thermus thermophilus), MjPgi (derived from Methoanococcus jannaschii), PfPgi (derived from Pyrococcus furiosus), Ap0768 (derived from Aeropyrum pernix), and Cl1150 (derived from Caldisphaera lagunensis).

24. The method of claim 23 wherein the phosphoglucoisomerase is selected from the group consisting of PfPgi (derived from Pyrococcus furiosus), Ap0768 (derived from Aeropyrum pernix), and Cl1150 (derived from Caldisphaera lagunensis).

25. The method of claim 23 wherein the phosphoglucoisomerase is selected from the group consisting of CtPgi (derived from Clostridium thermocellum), TtPgi (derived from Thermus thermophilus), and PfPgi (derived from Pyrococcus furiosus).

26. The method of claim 23 further comprising:
converting glucose 1-phosphate to glucose 6-phosphate using phosphoglucomutase.

27. The method of claim 26, wherein the phosphoglucomutase is selected from the group consisting of Tk1621 (derived from Thermococcus kodakaraensis), Pk02350 (derived from Pyrococcus kukulkanii), Af0458 (derived from Archaeoglobus fulgidus), CtPgm2 (derived from Clostridium thermocellum), TtPgm2 (derived from Thermus thermophilus), and TiManB (derived from Thermus islandicus).

28. The method of claim 26, wherein the phosphoglucomutase is selected from the group consisting of Tk1621 (derived from Thermococcus kodakaraensis), Pk02350 (derived from Pyrococcus kukulkanii) and CtPgm2 (derived from Clostridium thermocellum).

29. The method of claim 26 further comprising:
converting a polysaccharide to glucose 1-phosphate using an α-glucan phosphorylase.

30. The method of claim 29, wherein the α-glucan phosphorylase is selected from the group consisting of Tm08495 (derived from Thermotoga maritima), AaGlgP (derived from Aquifex aeolicus), TcGlgP (derived from Thermus caldophilus), PtAgp (derived from Pseudothermotoga thermarum), TzAgp (derived from Thermococcus zilligii), and PfAgp (derived from Pyrococcus furiosus).

31. The method of claim 29, wherein the α-glucan phosphorylase is selected from the group consisting of TzAgp (derived from Thermococcus zilligii) and PfAgp (derived from Pyrococcus furiosus).

32. The method of claim 29, wherein the α-glucan phosphorylase is selected from the group consisting of TzAgp (derived from Thermococcus zilligii), PtAgp (derived from Pseudothermotoga thermarum), and AaGlgP (derived from Aquifex aeolicus).

33. The method of claim 29, wherein the step of converting a polysaccharide to glucose 1-phosphate further comprises using a debranching enzyme.

34. The method of claim 33, wherein the debranching enzyme is a pullulanase, isoamylase, or a combination thereof.

35. The method of claim 34, wherein the pullulanase is selected from the group consisting of Fp1793 (derived from Fervidobacterium pennavorans), BfPull (derived from Bacillus flavocaldarius), and TRQ5Pul (derived from Thermotoga sp. RQ5).

36. The method of claim 34, wherein the isoamylase is selected from the group consisting of StTreX (derived from Sulfolobus tokodaii), MhTreX (derived from Metallosphaera hakonensis), and StGlgX (derived from Sphaerobacter thermophilus).

37. The method of claim 34, wherein the isoamylase is StTreX (derived from Sulfolobus tokodaii).

38. The method of claim 29, wherein the polysaccharide is maltodextrin, starch, glycogen, cellulose, or cellodextrin.

39. The method of claim 23 further comprising:
converting fructose 6-phosphate to allulose 6-phosphate using an allulose-6 phosphate epimerase.

40. The method of claim 39, wherein the allulose 6-phosphate epimerase is selected from the group consisting of BtAlsE (derived from Brevibacillus thermoruber), Thebr1340 (derived from Thermoanaerobacter brockii), CasuRpe2 (derived from Caldanaerobacter subterraneus), Defds2125 (derived from Deferribacter desulfuricans), Hg1285 (derived from Hydrogenivirga sp. 128-5-R1-1), Tthe1731 (derived from Thermoanerobacterium thermosaccharolyticum), Theru00510 (derived from Thermocrinis ruber), That0313 (derived from Thermosipho atlanticus), and ThtaRpe (derived from Thermosulfidibacter takaii).

41. The method of claim 39, wherein the allulose 6-phosphate epimerase is selected from the group consisting of BtAlsE (derived from Brevibacillus thermoruber), Hg1285 (derived from Hydrogenivirga sp. 128-5-R1-1), and Tthe1731 (derived from Thermoanerobacterium thermosaccharolyticum).

42. The method of claim 39 further comprising:
converting allulose 6-phosphate to allulose using an allulose-6 phosphate phosphatase.

43. The method of claim 42, wherein the allulose 6-phosphate phosphatase is selected from the group consisting of AaGph (derived from Aquifex aeolicus), Acel0099 (derived from Acidothermus cellulolyticus), CsPpaX (derived from Caldanaerobacter subterraneus), Cthe0261 (derived from Clostridium thermocellum), Desku1269 (derived from Desulfotomaculum kuznetsovii), Dgeo0096 (derived from Deinococcus geothermalis DSM 11300), Mth1760 (derived from Methanothermobacter thermautotrophicus), Pho459 (derived from Pyrococcus horikoshii Ot3), Pmob0141 (derived from Petrotoga mobilis), Tagg0346 (derived from Thermosphaera aggregans), Tfu0224 (derived from Thermobifida fusca), Theet2005 (derived from Thermoanaerobacter ethanolicus), Thein1775 (derived from Thermodesulfatator indicus), TheisHAD (derived from Thermus islandicus), Thewi1735 (derived from Thermoanaerobacter wiegelii), TtC1471 (derived from Thermus thermophilus), Vdis0326 (derived from Crenarchaeota archaeon 13_1_40CM_3_53_5), Ctn1320 (derived from Thermotoga neapolitana), Bf9343 (derived from Bacteroides fragilis), and Bvu4110 (derived from Bacteroides vulgatus).

44. The method of claim 42, wherein the allulose 6-phosphate phosphatase is selected from the group consisting of Cthe0261 (derived from Clostridium thermocellum), Bf9343 (derived from Bacteroides fragilis) and Bvu4110 (derived from Bacteroides vulgatus).

45. A cell-free method for producing allulose from a polysaccharide, the method comprising:
converting a polysaccharide to glucose 1-phosphate using an α-glucan phosphorylase;
converting glucose 1-phosphate to glucose 6-phosphate using phosphoglucomutase;
converting glucose 6-phosphate to fructose 6-phosphate using a phosphoglucoisomerase;
converting fructose 6-phosphate to allulose 6-phosphate using an allulose-6 phosphate epimerase; and
converting allulose 6-phosphate to allulose using an allulose-6 phosphate phosphatase.

46. The method of claim 45, wherein the α-glucan phosphorylase is selected from the group consisting of Tm08495 (derived from Thermotoga maritima), AaGlgP (derived from Aquifex aeolicus), TcGlgP (derived from Thermus caldophilus), PtAgp (derived from Pseudothermotoga thermarum), TzAgp (derived from Thermococcus zilligii), and PfAgp (derived from Pyrococcus furiosus).

47. The method of any one of claims 45 or 46 wherein the phosphoglucomutase is selected from the group consisting of Tk1621 (derived from Thermococcus kodakaraensis), Pk02350 (derived from Pyrococcus kukulkanii), Af0458 (derived from Archaeoglobus fulgidus), CtPgm2 (derived from Clostridium thermocellum), TtPgm2 (derived from Thermus thermophilus), and TiManB (derived from Thermus islandicus).

48. The method of any one of claims 45-47, wherein the phosphoglucoisomerase is selected from the group consisting of CtPgi (derived from Clostridium thermocellum), TtPgi (derived from Thermus thermophilus), MjPgi (derived from Methanococcus jannaschii), PfPgi (derived from Pyrococcus furiosus), Ap0768 (derived from Aeropyrum pernix), and Cl1150 (derived from Caldisphaera lagunensis).

49. The method of any one of claims 45-48, wherein the allulose 6-phosphate epimerase is selected from the group consisting of BtAlsE (derived from Brevibacillus thermoruber), Thebr1340 (derived from Thermoanaerobacter brockii), CasuRpe2 (derived from Caldanaerobacter subterraneus), Defds2125 (derived from Deferribacter desulfuricans), Hg1285 (derived from Hydrogenivirga sp. 128-5-R1-1), Tthe1731 (derived from Thermoanerobacterium thermosaccharolyticum), Theru00510 (derived from Thermocrinis ruber), That0313 (derived from Thermosipho atlanticus), and ThtaRpe (derived from Thermosulfidibacter takaii).

50. The method of any one of claims 45-49, wherein the allulose 6-phosphate phosphatase is selected from the group consisting of Bf9343 (derived from Bacteroides fragilis), Bvu4110 (derived from Bacteroides vulgatus), AaGph (derived from Aquifex aeolicus), Acel0099 (derived from Acidothermus cellulolyticus), CsPpaX (derived from Caldanaerobacter subterraneus), Cthe0261 (derived from Clostridium thermocellum), Desku1269 (derived from Desulfotomaculum kuznetsovii), Dgeo0096 (derived from Deinococcus geothermalis DSM 11300), Mth1760 (derived from Methanothermobacter thermautotrophicus), Pho459 (derived from Pyrococcus horikoshii Ot3), Pmob0141 (derived from Petrotoga mobilis), Tagg0346 (derived from Thermosphaera aggregans), Tfu0224 (derived from Thermobifida fusca), Theet2005 (derived from Thermoanaerobacter ethanolicus), Thein1775 (derived from Thermodesulfatator indicus), TheisHAD (derived from Thermus islandicus), Thewi1735 (derived from Thermoanaerobacter wiegelii), TtC1471 (derived from Thermus thermophilus), Vdis0326 (derived from Crenarchaeota archaeon 13_1_40CM_3_53_5), and Ctn1320 (derived from Thermotoga neapolitana).

51. The method of any one of claims 45-50, wherein the α-glucan phosphorylase is selected from the group consisting of TzAgp (derived from Thermococcus zilligii), AaGlgP (derived from Aquifex aeolicus), and PtAgp (derived from Pseudothermotoga thermarum).

52. The method of any one of claims 45-51, wherein the phosphoglucomutase is selected from the group consisting of Tk1621 (derived from Thermococcus kodakaraensis), Pk02350 (derived from Pyrococcus kukulkanii), and CtPgm2 (derived from Clostridium thermocellum).

53. The method of any one of claims 45-52, wherein the phosphoglucoisomerase is selected from the group consisting of CtPgi (derived from Clostridium thermocellum), TtPgi (derived from Thermus thermophilus) and PfPgi (derived from Pyrococcus furiosus).

54. The method of any one of claims 45-53, wherein the allulose 6-phosphate epimerase is selected from the group consisting of BtAlsE (derived from Brevibacillus thermoruber), Hg1285 (derived from Hydrogenivirga sp. 128-5-R1-1), Tthe1731 (derived from Thermoanerobacterium thermosaccharolyticum).

55. The method of any one of claims 45-54, wherein the allulose 6-phosphate phosphatase is selected from the group consisting of Cthe0261 (derived from Clostridium thermocellum), Bf9343 (derived from Bacteroides fragilis) and Bvu4110 (derived from Bacteroides vulgatus).

56. The method of any one of claims 45-50 and 52-55, wherein the α-glucan phosphorylase is selected from the group consisting of PfAgp (derived from Pyrococcus furiosus), and TzAgp (derived from Thermococcus zilligii).

57. The method of any one of claims 45-52 and 54-56, wherein the phosphoglucoisomerase is selected from the group consisting of CtPgi (derived from Clostridium thermocellum), TtPgi (derived from Thermus thermophilus), and PfPgi (derived from Pyrococcus furiosus).

58. The method of any one of claims 45-57, wherein the step of converting a polysaccharide to glucose 1-phosphate further comprises using a debranching enzyme.

59. The method of claim 58 wherein:
the debranching enzyme is selected from a pullulanase, isoamylase, or a combination thereof.

60. The method of any one of claims 58-59, wherein the debranching enzyme is a pullulanase, isoamylase, or a combination thereof selected from the group consisting of Fp1793 (derived from Fervidobacterium pennavorans), BfPull (derived from Bacillus flavocaldarius), TRQ5Pul (derived from Thermotoga sp. RQ5), StTreX (derived from Sulfolobus tokodaii), MhTreX (derived from Metallosphaera hakonensis), and StGlgX (derived from Sphaerobacter thermophilus).

61. The method of any one of claims 58-60, wherein the debranching enzyme is an isoamylase selected from the group consisting of StTreX (derived from Sulfolobus tokodaii), MhTreX (derived from Metallosphaera hakonensis), and StGlgX (derived from Sphaerobacter thermophilus).

62. The method of any one of claims 58-61, wherein the debranching enzyme is StTreX (derived from Sulfolobus tokodaii).

63. The method of any one of claims 45-62, wherein the polysaccharide is maltodextrin, starch, glycogen, or cellodextrin.

64. Allulose produced by the method of any one of claims 1-63.

65. A cell lysate comprising:
an α-glucan phosphorylase selected from the group consisting of TzAgp (derived from Thermococcus zilligii), AaGlgP (derived from Aquifex aeolicus), PtAgp (derived from Pseudothermotoga thermarum), and PfAgp (derived from Pyrococcus furiosus);
a phosphoglucomutase;
a phosphoglucoisomerase;
an allulose 6-phosphate epimerase; and
an allulose 6-phosphate phosphatase; and
optionally a debranching enzyme.

66. A cell lysate comprising:
a phosphoglucoisomerase selected from the group consisting of PfPgi (derived from Pyrococcus furiosus), CtPgi (derived from Clostridium thermocellum), TtPgi (derived from Thermus thermophilus)Ap0768 (derived from Aeropyrum pernix), and Cl1150 (derived from Caldisphaera lagunensis);
an α-glucan phosphorylase;
a phosphoglucomutase;
an allulose 6-phosphate epimerase;
an allulose 6-phosphate phosphatase; and
optionally a debranching enzyme.

67. An engineered cell comprising:
an α-glucan phosphorylase selected from the group consisting of TzAgp (derived from Thermococcus zilligii), AaGlgP (derived from Aquifex aeolicus), PtAgp (derived from Pseudothermotoga thermarum), and PfAgp (derived from Pyrococcus furiosus);
a phosphoglucomutase;
a phosphoglucoisomerase;
an allulose 6-phosphate epimerase; and
an allulose 6-phosphate phosphatase; and
optionally a debranching enzyme.

68. An engineered cell comprising:
a phosphoglucoisomerase selected from the group consisting of PfPgi (derived from Pyrococcus furiosus), CtPgi (derived from Clostridium thermocellum), TtPgi (derived from Thermus thermophilus) Ap0768 (derived from Aeropyrum pernix), and Cl1150 (derived from Caldisphaera lagunensis);
an α-glucan phosphorylase;
a phosphoglucomutase;
an allulose 6-phosphate epimerase;
an allulose 6-phosphate phosphatase; and
optionally a debranching enzyme.

69. The engineered cell of any of claims 67-68, wherein the cell is a bacterial cell or a yeast cell.

70. A mixture of cell lysates obtained from at least two cell populations, wherein the cells of each cell population express at least one or more enzymes selected from the group consisting of:
α-glucan phosphorylases selected from the group consisting of TzAgp (derived from Thermococcus zilligii), AaGlgP (derived from Aquifex aeolicus), PtAgp (derived from Pseudothermotoga thermarum), and PfAgp (derived from Pyrococcus furiosus);
phosphoglucomutases;
phosphoglucoisomerases;
allulose 6-phosphate epimerases;
allulose 6-phosphate phosphatases; and
debranching enzymes.

71. A mixture of cell lysates obtained from at least two cell populations, wherein the cells of each cell population express at least one or more enzymes selected from the group consisting of:
phosphoglucoisomerases selected from the group consisting of PfPgi (derived from Pyrococcus furiosus), CtPgi (derived from Clostridium thermocellum), TtPgi (derived from Thermus thermophilus) Ap0768 (derived from Aeropyrum pernix), and Cl1150 (derived from Caldisphaera lagunensis);
α-glucan phosphorylases;
phosphoglucomutases;
allulose 6-phosphate epimerases;
allulose 6-phosphate phosphatases; and
debranching enzymes.

72. A reaction mixture of cell lysates comprising:
an α-glucan phosphorylase selected from the group consisting of TzAgp (derived from Thermococcus zilligii), AaGlgP (derived from Aquifex aeolicus), PtAgp (derived from Pseudothermotoga thermarum), and PfAgp (derived from Pyrococcus furiosus);
a phosphoglucomutase;
a phosphoglucoisomerase;
an allulose 6-phosphate epimerase;
an allulose 6-phosphate phosphatase;
optionally a debranching enzyme;
a polysaccharide;
glucose 1-phosphate;
glucose 6-phosphate;
fructose 6-phosphate;
allulose 6-phosphate; and
allulose.

73. A reaction mixture of cell lysates comprising:
an α-glucan phosphorylase;
a phosphoglucomutase;
a phosphoglucoisomerase selected from the group consisting of PfPgi (derived from Pyrococcus furiosus), CtPgi (derived from Clostridium thermocellum), TtPgi (derived from Thermus thermophilus) Ap0768 (derived from Aeropyrum pernix), and Cl1150 (derived from Caldisphaera lagunensis);
an allulose 6-phosphate epimerase;
an allulose 6-phosphate phosphatase;
optionally a debranching enzyme;
a polysaccharide;
glucose 1-phosphate;
glucose 6-phosphate;
fructose 6-phosphate;
allulose 6-phosphate; and
allulose.

74. A cell lysate mixture comprising:
an α-glucan phosphorylase selected from the group consisting of TzAgp (derived from Thermococcus zilligii), AaGlgP (derived from Aquifex aeolicus), PtAgp (derived from Pseudothermotoga thermarum), and PfAgp (derived from Pyrococcus furiosus);
a phosphoglucomutase;
a phosphoglucoisomerase;
an allulose 6-phosphate epimerase;
an allulose 6-phosphate phosphatase;
optionally a debranching enzyme;
a polysaccharide;
glucose 1-phosphate;
glucose 6-phosphate;
fructose 6-phosphate;
allulose 6-phosphate; and
allulose for the synthesis of allulose.

75. A cell lysate mixture comprising:
an α-glucan phosphorylase;
a phosphoglucomutase;
a phosphoglucoisomerase selected from the group consisting of PfPgi (derived from Pyrococcus furiosus), CtPgi (derived from Clostridium thermocellum), TtPgi (derived from Thermus thermophilus) Ap0768 (derived from Aeropyrum pernix), and Cl1150 (derived from Caldisphaera lagunensis);
an allulose 6-phosphate epimerase;
an allulose 6-phosphate phosphatase;
optionally a debranching enzyme;
a polymeric glucose;
glucose 1-phosphate;
glucose 6-phosphate;
fructose 6-phosphate;
allulose 6-phosphate; and
allulose for the synthesis of allulose.

76. A cell-free method for producing allulose, the method comprising:
(a) culturing cells engineered to express a thermostable α-glucan phosphorylase selected from the group consisting of TzAgp (derived from Thermococcus zilligii), AaGlgP (derived from Aquifex aeolicus), PtAgp (derived from Pseudothermotoga thermarum), and PfAgp (derived from Pyrococcus furiosus),
a thermostable phosphoglucomutase, a thermostable phosphoglucoisomerase, a thermostable allulose 6-phosphate epimerase, and a thermostable allulose 6-phosphate phosphatase to produce cultured cells that express the thermostable enzymes;
(b) lysing the cultured cells to produce a cell lysate;
(c) heating the cell lysate to a temperature that inactivates native enzymatic activity but does not inactivate the thermostable enzymes of step (a) to produce a heat-inactivated lysate; and
(d) incubating the heat-inactivated lysate in the presence of a starch, glycogen, or any partially hydrolyzed derivative thereof and inorganic phosphate to produce allulose.

77. A cell-free method for producing allulose, the method comprising:
(a) culturing cells engineered to express a thermostable α-glucan phosphorylase, a thermostable phosphoglucomutase, a thermostable allulose 6-phosphate epimerase, a thermostable allulose 6-phosphate phosphatase, and
a thermostable phosphoglucoisomerase selected from the group consisting of PfPgi (derived from Pyrococcus furiosus), CtPgi (derived from Clostridium thermocellum), TtPgi (derived from Thermus thermophilus) Ap0768 (derived from Aeropyrum pernix), and Cl1150 (derived from Caldisphaera lagunensis),
to produce cultured cells that express the thermostable enzymes;
(b) lysing the cultured cells to produce a cell lysate;
(c) heating the cell lysate to a temperature that inactivates native enzymatic activity but does not inactivate the thermostable enzymes of step (a) to produce a heat-inactivated lysate; and
(d) incubating the heat-inactivated lysate in the presence of a starch, glycogen, or any partially hydrolyzed derivative thereof and inorganic phosphate to produce allulose.

78. A cell-free method for producing allulose, the method comprising:
(a) culturing at least two cell populations, wherein cells of each population are engineered to express at least one enzyme selected from the group consisting of:
thermostable α-glucan phosphorylases selected from the group consisting of TzAgp (derived from Thermococcus zilligii), AaGlgP (derived from Aquifex aeolicus), PtAgp (derived from Pseudothermotoga thermarum), and PfAgp (derived from Pyrococcus furiosus),
thermostable phosphoglucomutases, thermostable phosphoglucoisomerases, thermostable allulose 6-phosphate epimerases, and thermostable allulose 6-phosphate phosphatases, and thermostable debranching enzymes to produce at least two cultured populations of cells expressing different enzymes;
(b) lysing cells of the at least two cultured populations to produce at least two cell lysates;
(c) combining the at least two cell lysates to produce a cell lysate mixture that comprises a thermostable α-glucan phosphorylase, a thermostable phosphoglucomutase, a thermostable phosphoglucoisomerase, a thermostable allulose 6-phosphate epimerase, and a thermostable allulose 6-phosphate phosphatase, and optionally a thermostable debranching enzyme;
(d) heating the cell lysate mixture to a temperature that inactivates native enzymatic activity but does not inactivate the thermostable enzymes of step (c) to produce a heat-inactivated lysate; and
(e) incubating the reaction mixture in the presence of a starch, glycogen, or any partially hydrolyzed derivative thereof and inorganic phosphate to produce allulose.

79. A cell-free method for producing allulose, the method comprising:
(a) culturing at least two cell populations, wherein cells of each population are engineered to express at least one enzyme selected from the group consisting of:
thermostable α-glucan phosphorylases, thermostable phosphoglucomutases, thermostable allulose 6-phosphate epimerases, thermostable allulose 6-phosphate phosphatases, phosphoglucoisomerases selected from the group consisting of PfPgi (derived from Pyrococcus furiosus), CtPgi (derived from Clostridium thermocellum), TtPgi (derived from Thermus thermophilus) Ap0768 (derived from Aeropyrum pernix), and Cl1150 (derived from Caldisphaera lagunensis),
and optionally debranching enzymes to produce at least two cultured populations of cells expressing different enzymes;
(b) lysing cells of the at least two cultured populations to produce at least two cell lysates;
(c) combining the at least two cell lysates to produce a cell lysate mixture that comprises a thermostable α-glucan phosphorylase, a thermostable phosphoglucomutase, a thermostable phosphoglucoisomerase, a thermostable allulose 6-phosphate epimerase, and a thermostable allulose 6-phosphate phosphatase, and optionally a debranching enzyme;
(d) heating the cell lysate mixture to a temperature that inactivates native enzymatic activity but does not inactivate the enzymes of step (c) to produce a heat-inactivated lysate; and
(e) incubating the reaction mixture in the presence of a starch, glycogen, or any partially hydrolyzed derivative thereof and inorganic phosphate to produce allulose.

80. A cell-free method for producing allulose, the method comprising:
(a) culturing at least two cell populations, wherein cells of each population are engineered to express at least one enzyme selected from the group consisting of:
α-glucan phosphorylases selected from the group consisting of TzAgp (derived from Thermococcus zilligii), AaGlgP (derived from Aquifex aeolicus), PtAgp (derived from Pseudothermotoga thermarum), and PfAgp (derived from Pyrococcus furiosus),
phosphoglucomutases, phosphoglucoisomerases, allulose 6-phosphate epimerases, and allulose 6-phosphate phosphatases, and debranching enzymes to produce at least two cultured populations of cells expressing different enzymes wherein at least one of the foregoing enzymes is thermostable;
(b) lysing cells of the at least two cultured populations to produce at least two cell lysates;
(c) optionally heating one or more of the cell lysates of step (b) to a temperature that inactivates native enzymatic activity but does not inactivate the enzymes of step (a) to produce a heat-inactivated lysate;
(d) combining the cell lysates of step (b) and (c) to produce a cell lysate mixture that comprises an α-glucan phosphorylase, a phosphoglucomutase, a phosphoglucoisomerase, an allulose 6-phosphate epimerase, and an allulose 6-phosphate phosphatase, and optionally a debranching enzyme, wherein at least one of the foregoing enzymes is thermostable; and
(e) incubating the cell lysate mixture in the presence of a starch, glycogen, or any partially hydrolyzed derivative thereof and inorganic phosphate to produce allulose.

81. A cell-free method for producing allulose, the method comprising:
(a) culturing at least two cell populations, wherein cells of each population are engineered to express at least one enzyme selected from the group consisting of:
α-glucan phosphorylases, phosphoglucomutases, allulose 6-phosphate epimerases, allulose 6-phosphate phosphatases, a phosphoglucoisomerase selected from the group consisting of PfPgi (derived from Pyrococcus furiosus), CtPgi (derived from Clostridium thermocellum), TtPgi (derived from Thermus thermophilus) Ap0768 (derived from Aeropyrum pernix), and Cl1150 (derived from Caldisphaera lagunensis),
and optionally debranching enzymes to produce at least two cultured populations of cells expressing different enzymes wherein at least one of the foregoing enzymes is thermostable;
(b) lysing cells of the at least two cultured populations to produce at least two cell lysates;
(c) optionally heating one or more of the cell lysates of step (b) to a temperature that inactivates native enzymatic activity but does not inactivate the enzymes of step (a) to produce a heat-inactivated lysate;
(d) combining the cell lysates of step (b) and (c) to produce a cell lysate mixture that comprises an α-glucan phosphorylase, a phosphoglucomutase, a phosphoglucoisomerase, an allulose 6-phosphate epimerase, and an allulose 6-phosphate phosphatase, and optionally a debranching enzyme, wherein at least one of the foregoing enzymes is thermostable; and
(e) incubating the cell lysate mixture in the presence of a starch, glycogen, or any partially hydrolyzed derivative thereof and inorganic phosphate to produce allulose.

82. The method of any one of claims 76-81, wherein the cells comprise bacterial cells.

83. The method of any one of claims 76-81, wherein the cells comprise yeast cells.

84. The method for any one of claims 76-83, wherein at least one of the enzymes is heterologous to the cells.


SEQUENCE LISTING (10 sequences)

SEQ ID NO: 1 - protein, 26 aa, Artificial Sequence
  Synthetic Polypeptide
MKIKTGARILALSALTTMMFSASALA

SEQ ID NO: 2 - protein, 21 aa, Artificial Sequence
  Synthetic Polypeptide
MKQSTIALALLPLLFTPVTKA

SEQ ID NO: 3 - protein, 25 aa, Artificial Sequence
  Synthetic Polypeptide
MMITLRKLPLAVAVAAGVMSAQAMA

SEQ ID NO: 4 - protein, 23 aa, Artificial Sequence
  Synthetic Polypeptide
MNKKVLTLSAVMASMLFGAAAHA

SEQ ID NO: 5 - protein, 22 aa, Artificial Sequence
  Synthetic Polypeptide
MKYLLPTAAAGLLLLAAQPAMA

SEQ ID NO: 6 - protein, 19 aa, Artificial Sequence
  Synthetic Polypeptide
MKKIWLALAGLVLAFSASA

SEQ ID NO: 7 - protein, 23 aa, Artificial Sequence
  Synthetic Polypeptide
MMTKIKLLMLIIFYLIISASAHA

SEQ ID NO: 8 - protein, 21 aa, Artificial Sequence
  Synthetic Polypeptide
MKQALRVAFGFLILWASVLHA

SEQ ID NO: 9 - protein, 18 aa, Artificial Sequence
  Synthetic Polypeptide
MRVLLFLLLSLFMLPAFS

SEQ ID NO: 10 - protein, 39 aa, Artificial Sequence
  Synthetic Polypeptide
MANNDLFQASRRRFLAQLGGLTVAGMLGPSLLTPRRATA